(19) United States
(12) Patent Application Publication
(10) Pub. No.: US 2005/0261273 A1
(43) Pub. Date: Nov. 24, 2005

(54) Substituted urea and carbamate, phenacyl-2-hydroxy-3-diaminoalkane, and benzamide-2-hydroxy-3-diaminoalkane aspartyl-protease inhibitors

(76) Inventors: Varghese John, San Francisco, CA (US); Michel Maillard, Redwood City, CA (US); John Tucker, San Diego, CA (US); Jose Aquino, Daly City, CA (US); Roy Hom, San Francisco, CA (US); Jay S. Tung, Belmont, CA (US); Darren Dressen, Fremont, CA (US); Neerav Shah, San Mateo, CA (US); R. Jeffrey Neitz, San Francisco, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/075,292

(22) Filed: Mar. 9, 2005

Related U.S. Application Data
(60) Provisional application No. 60/551,192, filed on Mar. 9, 2004.
(60) Provisional application No. 60/575,829, filed on Jun. 2, 2004.
(60) Provisional application No. 60/591,857, filed on Jul. 29, 2004.
(60) Provisional application No. 60/622,589, filed on Oct. 28, 2004.

(51) Int. Cl. A61K 31/553; A61K 31/541; A61K 31/5377; A61K 31/496; A61K 31/445; A61K 31/40

(52) U.S. Cl. 514/211.01; 514/227.5; 514/237.2; 514/252.12; 514/317; 514/408

(57) ABSTRACT

The invention relates to acetyl 2-hydroxy-1,3-diaminospirocyclohexanes and derivatives thereof that are useful in treating at least one disease, disorder, and condition associated with amyloidosis. Amyloidosis refers to a collection of diseases, disorders, and condition associated with abnormal deposition of A-beta protein.

### CROSS-REFERENCE TO RELATED APPLICATIONS

This application claims the benefit of priority under 35 U.S.C. § 119(e) to U.S. Provisional Application 60/551,192, filed Mar. 9, 2004, U.S. Provisional Application 60/575,829, filed Jun. 2, 2004, U.S. Provisional Application 60/591,857, filed July 29, 2004, and U.S. Provisional Application 60/622,589, filed Oct. 28, 2004, incorporated herein by reference in full.

### FIELD OF THE PRESENT INVENTION

The present invention is directed to novel compounds of formula (I) and also to methods of treating at least one condition, disorder, or disease associated with amyloidosis.

### BACKGROUND OF THE PRESENT INVENTION

Amyloidosis refers to a collection of conditions, disorders, and diseases associated with abnormal deposition of amyloidal protein. For instance, Alzheimer's disease is believed to be caused by abnormal deposition of amyloidal protein in the brain. Thus, these amyloidal protein deposits, otherwise known as amyloid-beta peptide, A-beta, or betaA4, are the result of proteolytic cleavage of the amyloid precursor protein (APP).

The majority of APP molecules that undergo proteolytic cleavage are cleaved by the aspartyl protease alpha-secretase. Alpha-secretase cleaves APP between Lys687 and Leu688 producing a large, soluble fragment, alpha-sAPP, which is a secreted form of APP that does not result in beta-amyloid plaque formation. The alpha-secretase cleavage pathway precludes the formation of A-beta, thus providing an alternate target for preventing or treating amyloidosis.

Some APP molecules, however, are cleaved by a different aspartyl protease known as beta-secretase which is also referred to in the literature as BACE, BACE1, Asp2, and Memapsin2. Beta-secretase cleaves APP after Met671, creating a C-terminal fragment. See, for example, Sinha et al., Nature, (1999), 402:537-554 and published PCT application WO 00/17369. After cleavage of APP by beta-secretase, an additional aspartyl protease, gamma-secretase, may then cleave the C-terminus of this fragment, at either Val711 or Ile713, (found within the APP transmembrane domain), generating an A-beta peptide. The A-beta peptide may then proceed to form beta-amyloid plaques. A detailed description of the proteolytic processing of APP fragments is found, for example, in U.S. Pat. Nos. 5,441,870, 5,721,130, and 5,942,400.

The amyloidal disease Alzheimer's is a progressive degenerative disease that is characterized by two major pathologic observations in the brain which are (1) neurofibrillary tangles, and (2) beta-amyloid (or neuritic) plaques. A major factor in the development of Alzheimer's disease is A-beta deposits in regions of the brain responsible for cognitive activities. These regions include, for example, the hippocampus and cerebral cortex. A-beta is a neurotoxin that may be causally related to neuronal death observed in Alzheimer's disease patients. See, for example, Selkoe, Neuron, 6 (1991) 487. Since A-beta peptide accumulates as a result of APP processing by beta-secretase, inhibiting beta-secretase's activity is desirable for the treatment of Alzheimer's disease.

Dementia-characterized disorders also arise from A-beta accumulation in the brain including accumulation in cerebral blood vessels (known as vasculary amyloid angiopathy) such as in the walls of meningeal and parenchymal arterioles, small arteries, capillaries, and venules. A-beta may also be found in cerebrospinal fluid of both individuals with and without Alzheimer's disease. Additionally, neurofibrillary tangles similar to the ones observed in Alzheimer's patients can also be found in individuals without Alzheimer's disease. In this regard, a patient exhibiting symptoms of Alzheimer's due to A-beta deposits and neurofibrillary tangles in their cerebrospinal fluid may in fact be suffering from some other form of dementia. See, for example, Seubert et al., Nature, 359 (1992) 325-327. Examples of other forms of dementia where A-beta accumulation generates amyloidogenic plaques or results in vascular amyloid angiopathy include Trisomy 21 (Down's Syndrome), Hereditary Cerebral Hemorrhage with amyloidosis of the Dutch-Type (HCHWA-D), and other neurodegenerative disorders. Consequently, inhibiting beta-secretase is not only desirable for the treatment of Alzheimer's, but also for the treatment of other conditions associated with amyloidosis.

Amyloidosis is also implicated in the pathophysiology of stroke. Cerebral amyloid angiopathy is a common feature of the brains of stroke patients exhibiting symptoms of dementia, focal neurological syndromes, or other signs of brain damage. See, for example, Corio et al., Neuropath Appl. Neurobiol., 22 (1996) 216-227. This suggests that production and deposition of A-beta may contribute to the pathology of Alzheimer's disease, stroke, and other diseases and conditions associated with amyloidosis. Accordingly, the inhibition of A-beta production is desirable for the treatment of Alzheimer's disease, stroke, and other diseases and conditions associated with amyloidosis.

Presently there are no known effective treatments for preventing, delaying, halting, or reversing the progression of Alzheimer's disease and other conditions associated with amyloidosis. Consequently, there is an urgent need for methods of treatment capable of preventing and treating conditions associated with amyloidosis including Alzheimer's disease.

Likewise, there is a need for methods of treatment using compounds that inhibit beta-secretase-mediated cleavage of APP. There is also a need for methods of treatment using compounds that are effective inhibitors of A-beta production, and/or are effective at reducing A-beta deposits or plaques, as well as methods of treatment capable of combating diseases and conditions characterized by amyloidosis, or A-beta deposits, or plaques.

There is also a need for methods of treating conditions associated with amyloidosis using compounds that are efficacious, bioavailable and/or selective for beta-secretase. An increase in efficacy, selectivity, and/or oral bioavailability may result in preferred, safer, less expensive products that are easier for patients to use.

There is also a need for methods of treating conditions associated with amyloidosis using compounds with characteristics that would allow them to cross the blood-brain-barrier. Desirable characteristics include a low molecular weight and a high log P (increased log P=increased lipophilicity).

Generally, known aspartyl protease inhibitors are either incapable of crossing the blood-brain barrier or do so with great difficulty. These compounds are unsuitable for the treatment of the conditions described herein. Accordingly, there is a need for methods of treating conditions associated with amyloidosis using compounds that can readily cross the blood-brain barrier and inhibit beta-secretase.

There is also a need for a method of finding suitable compounds for inhibiting beta-secretase activity, inhibiting cleavage of APP, inhibiting production of A-beta, and/or reducing A-beta deposits or plaques.

The present invention is directed to novel compounds and also to methods of treating at least one condition, disorder, or disease associated with amyloidosis. An embodiment of the present invention is a method of administering at least one compound of formula (I), 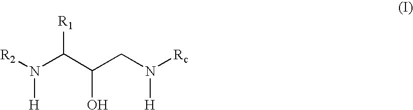
or a pharmaceutically acceptable salt thereof, wherein R₁, R₂, and R_{C} are defined below, in treating at least one condition, disorder, or disease associated with amyloidosis. Another embodiment of the present invention is directed to methods of treatment comprising administering at least one compound of formula (I), or a pharmaceutically acceptable salt thereof, wherein R₁, R₂, and R_{C} are defined below, useful in preventing, delaying, halting, or reversing the progression of Alzheimer's disease.

Another embodiment of the present invention is directed to uses of beta-secretase inhibitors of at least one compound of formula (I), or a pharmaceutically acceptable salt thereof, wherein R₁, R₂, and R_{C} are defined below, in treating or preventing at least one condition, disorder, or disease associated with amyloidosis.

Another embodiment of the present invention is to administer beta-secretase inhibitors of at least one compound of formula (I), or a pharmaceutically acceptable salt thereof, wherein R₁, R₂, and R_{C} are defined below, exhibiting at least one property chosen from improved efficacy, bioavailability, selectivity, and blood-brain barrier penetrating properties. The present invention accomplishes these objectives and provides further related advantages.

### BRIEF SUMMARY OF THE PRESENT INVENTION

The present invention is directed to novel compounds and also to methods of treating at least one condition, disorder, or disease associated with amyloidosis. As previously noted, amyloidosis refers to a collection of diseases, disorders, and conditions associated with abnormal deposition of A-beta protein.

An embodiment of the present invention is to provide compounds having properties contributing to viable pharmaceutical compositions. These properties include improved efficacy, bioavailability, selectivity, and/or blood-brain barrier penetrating properties. They can be inter-related, though an increase in any one of them correlates to a benefit for the compound and its corresponding method of treatment. For example, an increase in any one of these properties may result in preferred, safer, less expensive products that are easier for patients to use.

Accordingly, an embodiment of the present invention is to provide compounds of formula (I), 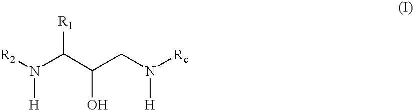
or pharmaceutically acceptable salts thereof, wherein R₁, R₂, and R_{C} are defined below.

Another embodiment of the present invention is a method of preventing or treating at least one condition that benefits from inhibition of at least one aspartyl-protease, comprising administering a composition comprising a therapeutically effective amount of at least one compound of formula (I): 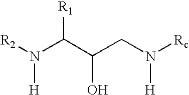
or a pharmaceutically acceptable salt thereof, wherein R₁, R₂, and R_{C} are as defined below.

In another embodiment, the present invention provides a method of preventing or treating at least one condition which benefits from inhibition of at least one aspartyl-protease, comprising administering to a host a composition comprising a therapeutically effective amount of at least one compound of the formula, 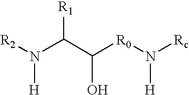
or a pharmaceutically acceptable salt thereof, and wherein R₁, R₂, and R_{C} are as defined below and R₀ is selected from —CH(alkyl)-, —C(alkyl)₂-, —CH(cycloalkyl)-, —C(alkyl)(cycloalkyl)-, and —C(cycloalkyl)₂—.

In another embodiment, the present invention provides a method for preventing or treating at least one condition associated with amyloidosis, comprising administering to a patient in need thereof a therapeutically effective amount of at least one compound of formula (I), or a pharmaceutically acceptable salt thereof, wherein the inhibition is at least 10% for a dose of 100 mg/kg or less, and wherein R₁, R₂, and R_{C} are as defined below.

In another embodiment, the present invention provides a method for preventing or treating at least one condition associated with amyloidosis, comprising administering to a patient in need thereof a therapeutically effective amount of at least one compound of formula (I), or a pharmaceutically acceptable salt thereof, the compound having an F value of at least 10%, wherein R₁, R₂, and R_{C} are as defined below.

In another embodiment, the present invention provides a method of preventing or treating at least one condition associated with amyloidosis, comprising administering to a host a composition comprising a therapeutically effective amount of at least one selective beta-secretase inhibitor of formula (I), or pharmaceutically acceptable salt thereof, wherein R₁, R₂, and R_{C} are as defined below.

In another embodiment, the present invention provides a method of preventing or treating Alzheimer's disease by administering to a host an effective amount of at least one compound of formula (I), or a pharmaceutically acceptable salt thereof, wherein R₁, R₂, and R_{C} are as defined below.

In another embodiment, the present invention provides a method of preventing or treating dementia by administering to a host an effective amount of at least one compound of formula (I), or pharmaceutically acceptable salt thereof, wherein R₁, R₂, and R_{C} are as defined below.

In another embodiment, the present invention provides a method of inhibiting beta-secretase activity in a host, the method comprising administering to the host an effective amount of at least one compound of formula (I), or a pharmaceutically acceptable salt thereof, wherein R₁, R₂, and R_{C} are as defined below.

In another embodiment, the present invention provides a method of inhibiting beta-secretase activity in a cell, the method comprising administering to the cell an effective amount of at least one compound of formula (I), or a pharmaceutically acceptable salt thereof, wherein R₁, R₂, and R_{C} are as defined below.

In another embodiment, the present invention provides a method of inhibiting beta-secretase activity in a host, the method comprising administering to the host an effective amount of at least one compound of formula (I), or a pharmaceutically acceptable salt thereof, wherein the host is a human, and wherein R₁, R₂, and R_{C} are as defined below.

In another embodiment, the present invention provides a method of affecting beta-secretase-mediated cleavage of amyloid precursor protein in a patient, comprising administering a therapeutically effective amount of at least one compound of formula (I), or a pharmaceutically acceptable salt thereof, wherein R₁, R₂, and R_{C} are as defined below.

In another embodiment, the present invention provides a method of inhibiting cleavage of amyloid precursor protein at a site between Met596 and Asp597 (numbered for the APP-695 amino acid isotype), or at a corresponding site of an isotype or mutant thereof, comprising administering a therapeutically effective amount of at least one compound of formula (I), or a pharmaceutically acceptable salt thereof, wherein R₁, R₂, and R_{C} are as defined below.

In another embodiment, the present invention provides a method of inhibiting production of A-beta, comprising administering to a patient a therapeutically effective amount of at least one compound of formula (I), or a pharmaceutically acceptable salt thereof, wherein R₁, R₂, and R_{C} are as defined below.

In another embodiment, the present invention provides a method of preventing or treating deposition of A-beta, comprising administering a therapeutically effective amount of at least one compound of formula (I), or a pharmaceutically acceptable salt thereof, wherein R₁, R₂, and R_{C} are as defined below.

In another embodiment, the present invention provides a method of preventing, delaying, halting, or reversing a disease characterized by A-beta deposits or plaques, comprising administering a therapeutically effective amount of at least one compound of formula (I), or a pharmaceutically acceptable salt thereof, wherein R₁, R₂, and R_{C} are as defined below.

In another embodiment, the A-beta deposits or plaques are in a human brain.

In another embodiment, the present invention provides a method of inhibiting the activity of at least one aspartyl protease in a patient in need thereof, comprising administering a therapeutically effective amount of at least one compound of formula (I), or a pharmaceutically acceptable salt thereof, wherein R₁, R₂, and R_{C} are as defined below.

In another embodiment, the at least one aspartyl protease is beta-secretase.

In another embodiment, the present invention provides a method of interacting an inhibitor with beta-secretase, comprising administering to a patient in need thereof a therapeutically effective amount of at least one compound of formula (I), or a pharmaceutically acceptable salt thereof, wherein R₁, R₂, and R_{C} are as defined below, wherein the at least one compound interacts with at least one beta-secretase subsite such as S1, S1′, or S2′.

In another embodiment, the present invention provides an article of manufacture, comprising (a) at least one dosage form of at least one compound of formula (I), or pharmaceutically acceptable salt thereof, wherein R₁, R₂, and R_{C} are defined below, (b) a package insert providing that a dosage form comprising a compound of formula (I) should be administered to a patient in need of therapy for at least one disorder, condition or disease associated with amyloidosis, and (c) at least one container in which at least one dosage form of at least one compound of formula (I) is stored.

In another embodiment, the present invention provides a packaged pharmaceutical composition for treating at least one condition related to amyloidosis, comprising (a) a container which holds an effective amount of at least one compound of formula (I), or a pharmaceutically acceptable salt thereof, wherein R₁, R₂, and R_{C} are as defined below, and (b) instructions for using the pharmaceutical composition.

### DEFINITIONS

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, it is to be understood that the substituents can be the same or different.

APP, amyloid precursor protein, is defined as any APP polypeptide, including APP variants, mutations, and isoforms, for example, as disclosed in U.S. Pat. No. 5,766,846.

Beta-amyloid peptide (A-beta peptide) is defined as any peptide resulting from beta-secretase mediated cleavage of APP, including, for example, peptides of 39, 40, 41, 42, and 43 amino acids, and extending from the beta-secretase cleavage site to amino acids 39, 40, 41, 42, or 43.

Beta-secretase is an aspartyl protease that mediates cleavage of APP at the N-terminus edge of A-beta. Human beta-secretase is described, for example, in WO 00/17369.

The term “complex” as used herein refers to an inhibitor-enzyme complex, wherein the inhibitor is a compound of formula (I) described herein and wherein the enzyme is beta-secretase or a fragment thereof.

The term “host” as used herein refers to a cell or tissue, in vitro or in vivo, an animal, or a human.

The term “treating” refers to administering a compound or a composition of formula (I) to a host having at least a tentative diagnosis of disease or condition. The methods of treatment and compounds of the present invention will delay, halt, or reverse the progression of the disease or condition thereby giving the host a longer and/or more functional life span.

The term “preventing” refers to administering a compound or a composition of formula (I) to a host who has not been diagnosed as having the disease or condition at the time of administration, but who could be expected to develop the disease or condition or be at increased risk for the disease or condition. The methods of treatment and compounds of the present invention may slow the development of disease symptoms, delay the onset of the disease or condition, halt the progression of disease development, or prevent the host from developing the disease or condition at all. Preventing also includes administration of at least one compound or a composition of the present invention to those hosts thought to be predisposed to the disease or condition due to age, familial history, genetic or chromosomal abnormalities, due to the presence of one or more biological markers for the disease or condition, such as a known genetic mutation of APP or APP cleavage products in brain tissues or fluids, and/or due to environmental factors.

The term “halogen” in the present invention refers to fluorine, bromine, chlorine, or iodine.

The term “alkyl” in the present invention refers to straight or branched chain alkyl groups having 1 to 20 carbon atoms. An alkyl group may optionally comprise at least one double bond and/or at least one triple bond. The alkyl groups herein are unsubstituted or substituted in one or more positions with various groups. For example, such alkyl groups may be optionally substituted with at least one group independently selected from alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like. Additionally, at least one carbon within any such alkyl may be optionally replaced with —C(O)—.

Examples of alkyls include methyl, ethyl, ethenyl, ethynyl, propyl, 1-ethyl-propyl, propenyl, propynyl, isopropyl, n-butyl, isobutyl, sec-butyl, tert-butyl, 2-methylbutyl, 3-methyl-butyl, 1-but-3-enyl, butynyl, pentyl, 2-pentyl, isopentyl, neopentyl, 3-methylpentyl, 1-pent-3-enyl, 1-pent-4-enyl, pentyn-2-yl, hexyl, 2-hexyl, 3-hexyl, 1-hex-5-enyl, formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1,-dimethyl-ethyl, 1,1-dimethyl-propyl, cyano-dimethyl-methyl, propylamino, and the like.

In an embodiment, alkyls may be selected from sec-butyl, isobutyl, ethynyl, 1-ethyl-propyl, pentyl, 3-methyl-butyl, pent-4-enyl, isopropyl, tert-butyl, 2-methylbutane, and the like.

In another embodiment, alkyls may be selected from formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1,-dimethyl-ethyl, 1,1-dimethyl-propyl, cyano-dimethyl-methyl, propylamino, and the like.

The term “alkoxy” in the present invention refers to straight or branched chain alkyl groups, wherein an alkyl group is as defined above, and having 1 to 20 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexyloxy, heptyloxy, allyloxy, 2-(2-methoxy-ethoxy)-ethoxy, benzyloxy, 3-methylpentoxy, and the like.

In an embodiment, alkoxy groups may be selected from allyloxy, hexyloxy, heptyloxy, 2-(2-methoxy-ethoxy)-ethoxy, benzyloxy, and the like.

The term “—C(O)-alkyl” or “alkanoyl” refers to an acyl radical derived from an alkylcarboxylic acid, a cycloalkylcarboxylic acid, a heterocycloalkylcarboxylic acid, an arylcarboxylic acid, an arylalkylcarboxylic acid, a heteroarylcarboxylic acid, or a heteroarylalkylcarboxylic acid, examples of which include formyl, acetyl, 2,2,2-trifluoroacetyl, propionyl, butyryl, valeryl, 4-methylvaleryl, and the like.

The term “cycloalkyl” refers to an optionally substituted carbocyclic ring system of one or more 3, 4, 5, 6, 7, or 8 membered rings, including 9, 10, 11, 12, 13, and 14 membered fused ring systems, all of which can be saturated or partially unsaturated. The cycloalkyl may be monocyclic, bicyclic, tricyclic, and the like. Bicyclic and tricyclic as used herein are intended to include both fused ring systems, such as adamantyl, octahydroindenyl, decahydro-naphthyl, and the like, substituted ring systems, such as cyclopentylcyclohexyl, and spirocycloalkyls such as spiro[2.5]octane, spiro[4.5]decane, 1,4-dioxa-spiro[4.5]decane, and the like. A cycloalkyl may optionally be a benzo fused ring system, which is optionally substituted as defined herein with respect to the definition of aryl. At least one —CH₂— group within any such cycloalkyl ring system may be optionally replaced with —C(O)—, —C(S)—, —C(═N—H)—, —C(═N—OH)—, —C(═N-alkyl)—(optionally substituted as defined herein with respect to the definition of alkyl), or —C(═N—O-alkyl)—(optionally substituted as defined herein with respect to the definition of alkyl).

Further examples of cycloalkyl radicals include cyclopropyl, cyclobutyl, cyclopentyl, cyclohexyl, octahydronaphthyl, 2,3-dihydro-1H-indenyl, and the like.

In an embodiment, a cycloalkyl may be selected from cyclopentyl, cyclohexyl, cycloheptyl, adamantenyl, bicyclo[2.2.1]heptyl, and the like.

The cycloalkyl groups herein are unsubstituted or substituted in at least one position with various groups. For example, such cycloalkyl groups may be optionally substituted with alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkylthio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like.

The term “cycloalkylcarbonyl” refers to an acyl radical of the formula cycloalkyl-C(O)— in which the term “cycloalkyl” has the significance given above, such as cyclopropylcarbonyl, cyclohexylcarbonyl, adamantylcarbonyl, 1,2,3,4-tetrahydro-2-naphthoyl, 2-acetamido-1,2,3,4-tetrahydro-2-naphthoyl, 1-hydroxy-1,2,3,4-tetrahydro-6-naphthoyl, and the like.

The term “heterocycloalkyl,” “heterocycle,” or “heterocyclyl,” refers to a monocyclic, bicyclic or tricyclic heterocycle radical, containing at least one nitrogen, oxygen or sulfur atom ring member and having 3 to 8 ring members in each ring, wherein at least one ring in the heterocycloalkyl ring system may optionally contain at least one double bond. At least one —CH₂— group within any such heterocycloalkyl ring system may be optionally replaced with —C(O)—, —C(S)—, —C(N)—, —C(═N—H)—, —C(═N—OH)—, —C(═N-alkyl)—(optionally substituted as defined herein with respect to the definition of alkyl), or —C(═N—O-alkyl)—(optionally substituted as defined herein with respect to the definition of alkyl).

The terms “bicyclic” and “tricyclic” as used herein are intended to include both fused ring systems, such as 2,3-dihydro-1H-indole, and substituted ring systems, such as bicyclohexyl. At least one —CH₂— group within any such heterocycloalkyl ring system may be optionally replaced with —C(O)—, —C(N)— or —C(S)—. Heterocycloalkyl is intended to include sulfones, sulfoxides, N-oxides of tertiary nitrogen ring members, and carbocyclic fused and benzo fused ring systems wherein the benzo fused ring system is optionally substituted as defined herein with respect to the definition of aryl. Such heterocycloalkyl radicals may be optionally substituted on one or more carbon atoms by halogen, alkyl, alkoxy, cyano, nitro, amino, alkylamino, dialkylamino, monoalkylaminoalkyl, dialkylaminoalkyl, haloalkyl, haloalkoxy, aminohydroxy, oxo, aryl, aralkyl, heteroaryl, heteroaralkyl, amidino, N-alkylamidino, alkoxycarbonylamino, alkylsulfonylamino, and the like, and/or on a secondary nitrogen atom (i.e., —NH—) by hydroxy, alkyl, aralkoxycarbonyl, alkanoyl, heteroaralkyl, phenyl, phenylalkyl, and the like.

Examples of a heterocycloalkyl include morpholinyl, thiomorpholinyl, thiomorpholinyl S-oxide, thiomorpholinyl S,S-dioxide, piperazinyl, homopiperazinyl, pyrrolidinyl, pyrrolinyl, 2,5-dihydro-pyrrolyl, tetrahydropyranyl, pyranyl, thiopyranyl, piperidinyl, tetrahydrofuranyl, tetrahydrothienyl, imidazolidinyl, homopiperidinyl, 1,2-dihyrdo-pyridinyl, homomorpholinyl, homothiomorpholinyl, homothiomorpholinyl S,S-dioxide, oxazolidinonyl, dihydropyrazolyl, dihydropyrrolyl, 1,4-dioxa-spiro[4.5]decyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide, 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonylpiperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

In an embodiment, a heterocycloalkyl may be selected from pyrrolidinyl, 2,5-dihydro-pyrrolyl, piperidinyl, 1,2-dihyrdo-pyridinyl, pyranyl, piperazinyl, imidazolidinyl, thiopyranyl, tetrahydropyranyl, 1,4-dioxa-spiro[4.5]decyl, and the like.

In another embodiment, a heterocycloalkyl may be selected from 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonyl piperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

The term “aryl” refers to an aromatic carbocyclic group having a single ring (e.g., phenyl) or multiple condensed rings in which at least one ring is aromatic. The aryl may be monocyclic, bicyclic, tricyclic, etc. Bicyclic and tricyclic as used herein are intended to include both fused ring systems, such as naphthyl and β-carbolinyl, and substituted ring systems, such as biphenyl, phenylpyridyl, diphenylpiperazinyl, tetrahydronaphthyl, and the like. Preferred aryl groups of the present invention are phenyl, 1-naphthyl, 2-naphthyl, indanyl, indenyl, dihydronaphthyl, fluorenyl, tetralinyl or 6,7,8,9-tetrahydro-5H-benzo[a]cycloheptenyl. The aryl groups herein are unsubstituted or substituted in one or more positions with various groups. For example, such aryl groups may be optionally substituted with alkyl, alkoxy, C(O)H, carboxy, alkoxycarbonyl, aryl, heteroaryl, cycloalkyl, heterocyclalkyl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, aralkoxycarbonylamino, haloalkyl, haloalkoxy, aminoalkyl, monoalkylaminoalkyl, dialkylaminoalkyl, and the like.

Examples of aryl radicals are phenyl, p-tolyl, 4-methoxyphenyl, 4-(tert-butoxy)phenyl, 3-methyl-4-methoxyphenyl, 4-CF₃-phenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, piperazinylphenyl, and the like.

Further examples of aryl radicals include 3-tert-butyl-1-fluoro-phenyl, 1,3-difluoro-phenyl, (1-hydroxy-1-methyl-ethyl)-phenyl, 1-fluoro-3-(2-hydroxy-1,1-dimethyl-ethyl)-phenyl, (1,1-dimethyl-propyl)-phenyl, cyclobutyl-phenyl, pyrrolidin-2-yl-phenyl, (5-oxo-pyrrolidin-2-yl)-phenyl, (2,5-dihydro-1H-pyrrol-2-yl)-phenyl, (1H-pyrrol-2-yl)-phenyl, (cyano-dimethyl-methyl)-phenyl, tert-butyl-phenyl, 1-fluoro-2-hydroxy-phenyl, 1,3-difluoro-4-propylamino-phenyl, 1,3-difluoro-4-hydroxy-phenyl, 1,3-difluoro-4-ethylamino-phenyl, 3-isopropyl-phenyl, (3H-[1,2,3]triazol-4-yl)-phenyl, [1,2,3]triazol-1-yl-phenyl, [1,2,4]thiadiazol-3-yl-phenyl, [1,2,4]thiadiazol-5-yl-phenyl, (4H-[1,2,4]triazol-3-yl)-phenyl, [1,2,4]oxadiazol-3-yl-phenyl, imidazol-1-yl-phenyl, (3H-imidazol-4-yl)-phenyl, [1,2,4]triazol-4-yl-phenyl, [1,2,4]oxadiazol-5-yl-phenyl, isoxazol-3-yl-phenyl, (1-methyl-cyclopropyl)-phenyl, isoxazol-4-yl-phenyl, isoxazol-5-yl-phenyl, 1-cyano-2-tert-butyl-phenyl, 1-trifluoromethyl-2-tert-butyl-phenyl, 1-chloro-2-tert-butyl-phenyl, 1-acetyl-2-tert-butyl-phenyl, 1-trifluoromethyl-2-methyl-phenyl, 1-tert-butyl-2-ethyl-phenyl,l 1-cyano-3-tert-butyl-phenyl, 1-trifluoromethyl-3-tert-butyl-phenyl, 1-chloro-3-tert-butyl-phenyl, 1-acetyl-3-tert-butyl-phenyl, 1-tert-butyl-3-methyl-phenyl 1-tert-butyl-3-ethyl-phenyl, 4-tert-butyl-1-imidazol-1-yl-phenyl, ethylphenyl, isobutylphenyl, isopropylphenyl, 3-allyloxy-1-fluoro-phenyl, (2,2-dimethyl-propyl)-phenyl, ethynylphenyl, 1-fluoro-3-heptyloxy-phenyl, 1-fluoro-3-[2-(2-methoxy-ethoxy)-ethoxy]-phenyl, 1-benzyloxy-3-fluoro-phenyl, 1-fluoro-3-hydroxy-phenyl, 1-fluoro-3-hexyloxy-phenyl, (4-methyl-thiophen-2-yl)-phenyl, (5-acetyl-thiophen-2-yl)-phenyl, furan-3-yl-phenyl, thiophen-3-yl-phenyl, (5-formyl-thiophen-2-yl)-phenyl, (3-formyl-furan-2-yl)-phenyl, acetylamino-phenyl, trifluoromethylphenyl, sec-butyl-phenyl, pentylphenyl, (3-methyl-butyl)-phenyl, (1-ethyl-propyl)-phenyl, cyclopentyl-phenyl, 3-pent-4-enyl-phenyl, phenyl propionic acid ethyl ester, pyridin-2-yl-phenyl, (3-methyl-pyridin-2-yl)-phenyl, thiazol-2-yl-phenyl, (3-methyl-thiophen-2-yl)-phenyl, fluoro-phenyl, adamantan-2-yl-phenyl, 1,3-difluoro-2-hydroxy-phenyl, cyclopropyl-phenyl, 1-bromo-3-tert-butyl-phenyl, (3-bromo-[1,2,4]thiadiazol-5-yl)-phenyl, (1-methyl-1H-imidazol-2-yl)-phenyl, 3,5-dimethyl-3H-pyrazol-4-yl)-phenyl, (3,6-dimethyl-pyrazin-2-yl)-phenyl, (3-cyano-pyrazin-2-yl)-phenyl, thiazol-4-yl-phenyl, (4-cyano-pyridin-2-yl)-phenyl, pyrazin-2-yl-phenyl, (6-methyl-pyridazin-3-yl)-phenyl, (2-cyano-thiophen-3-yl)-phenyl, (2-chloro-thiophen-3-yl)-phenyl, (5-acetyl-thiophen-3-yl)-phenyl, cyano-phenyl, and the like.

The term “heteroaryl” refers to an aromatic heterocycloalkyl radical as defined above. The heteroaryl groups herein are unsubstituted or substituted in at least one position with various groups. For example, such heteroaryl groups may be optionally substituted with, for example, alkyl, alkoxy, halogen, hydroxy, cyano, nitro, amino, monoalkylamino, dialkylamino, haloalkyl, haloalkoxy, C(O)H, carboxy, alkoxycarbonyl, cycloalkyl, heterocyclalkyl, aryl, heteroaryl, amido, alkanoylamino, amidino, alkoxycarbonylamino, N-alkyl amidino, N-alkyl amido, N,N′-dialkylamido, alkyl thio, alkylsulfinyl, alkylsulfonyl, aralkoxycarbonylamino, aminoalkyl, monoalkylaminoalkyl, dialkylaminoalkyl, and the like.

Examples of heteroaryl groups include pyridyl, pyrimidyl, furanyl, imidazolyl, thienyl, oxazolyl, thiazolyl, pyrazinyl, 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, quinolinyl, benzothienyl, indolyl, indolinyl, pyridazinyl, isoindolyl, isoquinolyl, quinazolinyl, quinoxalinyl, phthalazinyl, imidazolyl, isoxazolyl, pyrazolyl, indolizinyl, indazolyl, benzothiazolyl, benzimidazolyl, benzofuranyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, tetrahydrocarbazole, tetrahydrobetacarboline, and the like.

In an embodiment, a heteroaryl group may be selected from pyridyl, pyrimidyl, furanyl, imidazolyl, thienyl, oxazolyl, thiazolyl, pyrazinyl, and the like.

In another embodiment, a heteroaryl group may be selected from 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, and the like.

Further examples of heterocycloalkyls and heteroaryls may be found in Katritzky, A. R. et al., Comprehensive Heterocyclic Chemistry: The Structure, Reactions, Synthesis and Use of Heterocyclic Compounds, Vol. 1-8, New York: Pergamon Press, 1984.

The term “aralkoxycarbonyl” refers to a radical of the formula aralkyl-O—C(O)— in which the term “aralkyl” is encompassed by the definitions above for aryl and alkyl. Examples of an aralkoxycarbonyl radical include benzyloxycarbonyl 4-methoxyphenylmethoxycarbonyl, and the like.

The term “aryloxy” refers to a radical of the formula —O-aryl in which the term aryl is as defined above.

The term “aralkanoyl” refers to an acyl radical derived from an aryl-substituted alkanecarboxylic acid such as phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The term “aroyl” refers to an acyl radical derived from an arylcarboxylic acid, “aryl” having the meaning given above. Examples of such aroyl radicals include substituted and unsubstituted benzoyl or naphthoyl such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “haloalkyl” refers to an alkyl radical having the meaning as defined above wherein one or more hydrogens are replaced with a halogen. Examples of such haloalkyl radicals include chloromethyl, 1-bromoethyl, fluoromethyl, difluoromethyl, trifluoromethyl, 1,1,1-trifluoroethyl, and the like.

The term “epoxide” refers to chemical compounds or reagents comprising a bridging oxygen wherein the bridged atoms are also bonded to one another either directly or indirectly. Examples of epoxides include epoxyalkyl (e.g., ethylene oxide, and 1,2-epoxybutane), and epoxycycloalkyl (e.g., 1,2-epoxycyclohexane, 1,2-epoxy-1-methylcyclohexane), and the like.

The term “structural characteristics” refers to chemical moieties, chemical motifs, and portions of chemical compounds. These include R groups, such as but not limited to those defined herein, ligands, appendages, and the like. For example, structural characteristics may be defined by their properties, such as, but not limited to, their ability to participate in intermolecular interactions including Van der Waal's interactions (e.g., electrostatic interactions, dipole-dipole interactions, dispersion forces, hydrogen bonding, and the like). Such characteristics may impart desired pharmacokinetic properties and thus have an increased ability to cause the desired effect and thus prevent or treat the targeted diseases or conditions.

Compounds of formula (I) also comprise structural moieties that may participate in inhibitory interactions with at least one subsite of beta-secretase. For example, moieties of the compounds of formula (I) may interact with at least one of the S1, S1′ and S2′ subsites, wherein S1 comprises residues Leu30, Tyr71, Phe108, Ile110, and Trp115, S1 ′ comprises residues Tyr198, Ile226, Val227, Ser 229, and Thr231, and S2′ comprises residues Ser35, Asn37, Pro70, Tyr71, Ile118, and Arg128. Such compounds and methods of treatment may have an increased ability to cause the desired effect and thus prevent or treat the targeted diseases or conditions.

The term “pharmaceutically acceptable” refers to those properties and/or substances that are acceptable to the patient from a pharmacological/toxicological point of view, and to the manufacturing pharmaceutical chemist from a physical/chemical point of view regarding composition, formulation, stability, patient acceptance, and bioavailability.

The term “effective amount” as used herein refers to an amount of a therapeutic agent administered to a host, as defined herein, necessary to achieve a desired effect.

The term “therapeutically effective amount” as used herein refers to an amount of a therapeutic agent administered to a host to treat or prevent a condition treatable by administration of a composition of the invention. That amount is the amount sufficient to reduce or lessen at least one symptom of the disease being treated or to reduce or delay onset of one or more clinical markers or symptoms of the disease.

The term “therapeutically active agent” refers to a compound or composition that is administered to a host, either alone or in combination with another therapeutically active agent, to treat or prevent a condition treatable by administration of a composition of the invention.

The terms “pharmaceutically acceptable salt” and “salts thereof” refer to acid addition salts or base addition salts of the compounds in the present invention. A pharmaceutically acceptable salt is any salt which retains the activity of the parent compound and does not impart any deleterious or undesirable effect on the subject to whom it is administered and in the context in which it is administered. Pharmaceutically acceptable salts include salts of both inorganic and organic acids. Pharmaceutically acceptable salts include acid salts such as acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Other acceptable salts may be found, for example, in Stahl et al., Pharmaceutical Salts: Properties, Selection, and Use, Wiley-VCH; 1st edition (Jun. 15, 2002).

In an embodiment of the present invention, a pharmaceutically acceptable salt is selected from hydrochloric, hydrobromic, hydroiodic, nitric, sulfuric, phosphoric, citric, methanesulfonic, CH₃—(CH₂)₀₋₄—COOH, HOOC—(CH₂)₀₋₄—COOH, HOOC—CH═CH—COOH, phenyl-COOH, and the like.

The term “unit dosage form” refers to physically discrete units suitable as unitary dosages for human subjects or other mammals, each unit containing a predetermined quantity of active material calculated to produce the desired therapeutic effect, in association with a suitable pharmaceutical vehicle. The concentration of active compound in the drug composition will depend on absorption, inactivation, and/or excretion rates of the active compound, the dosage schedule, the amount administered and medium and method of administration, as well as other factors known to those of skill in the art.

The term “modulate” refers to a chemical compound's activity of either enhancing or inhibiting a functional property of biological activity or process.

The terms “interact” and “interactions” refer to a chemical compound's association and/or reaction with another chemical compound, such as an interaction between an inhibitor and beta-secretase. Interactions include, but are not limited to, hydrophobic, hydrophilic, lipophilic, lipophobic, electrostatic, and van der Waal's interactions including hydrogen bonding.

An “article of manufacture” as used herein refers to materials useful for the diagnosis, prevention or treatment of the disorders described above, such as a container with a label. The label can be associated with the article of manufacture in a variety of ways including, for example, the label may be on the container or the label may be in the container as a package insert. Suitable containers include, for example, blister packs, bottles, bags, vials, syringes, test tubes, and the like. The containers may be formed from a variety of materials such as glass, metal, plastic, rubber, paper, and the like. The container holds a composition as described herein which is effective for diagnosing, preventing, or treating a condition treatable by a compound or composition of the present invention.

The article of manufacture may contain bulk quantities or less of a composition as described herein. The label on, or associated with, the container may provide instructions for the use of the composition in diagnosing, preventing, or treating the condition of choice, instructions for the dosage amount and for the methods of administration. The label may further indicate that the composition is to be used in combination with one or more therapeutically active agents wherein the therapeutically active agent is selected from an antioxidant, an anti-inflammatory, a gamma-secretase inhibitor, a neurotrophic agent, an acetyl cholinesterase inhibitor, a statin, an A-beta, an anti-A-beta antibody, and/or a beta-secretase complex or fragment thereof. The article of manufacture may further comprise multiple containers, also referred to herein as a kit, comprising a therapeutically active agent or a pharmaceutically-acceptable buffer, such as phosphate-buffered saline, Ringer's solution and/or dextrose solution. It may further include other materials desirable from a commercial and user standpoint, including other buffers, diluents, filters, needles, syringes, and/or package inserts with instructions for use.

The compounds of formula (I), their compositions, and methods of treatment employing them, can be enclosed in multiple or single dose containers. The enclosed compounds and/or compositions can be provided in kits, optionally including component parts that can be assembled for use. For example, a compound inhibitor in lyophilized form and a suitable diluent may be provided as separated components for combination prior to use. A kit may include a compound inhibitor and at least one additional therapeutic agent for co-administration. The inhibitor and additional therapeutic agents may be provided as separate component parts.

A kit may include a plurality of containers, each container holding at least one unit dose of the compound of the present invention. The containers are preferably adapted for the desired mode of administration, including, for example, pill, tablet, capsule, powder, gel or gel capsule, sustained-release capsule, or elixir form, and/or combinations thereof, and the like for oral administration, depot products, pre-filled syringes, ampoules, vials, and the like for parenteral administration, and patches, medipads, creams, and the like for topical administration.

The term “Cₘₐₓ” refers to the peak plasma concentration of a compound in a host.

The term “Tₘₐₓ” refers to the time at peak plasma concentration of a compound in a host.

The term “half-life” refers to the period of time required for the concentration or amount of a compound in a host to be reduced to exactly one-half of a given concentration or amount.

### DETAILED DESCRIPTION OF THE PRESENT INVENTION

The present invention is directed to novel compounds and also to methods of treating conditions, disorders, and diseases associated with amyloidosis. Amyloidosis refers to a collection of diseases, disorders, and conditions associated with abnormal deposition of amyloidal protein.

An embodiment of the present invention provides methods of preventing or treating at least one condition associated with amyloidosis using compounds of formula (I) with a high degree of efficacy. Compounds and methods of treatment that are efficacious are those that have an increased ability to cause the desired effect and thus prevent or treat the targeted diseases or conditions.

Accordingly, another embodiment of the present invention provides a method of preventing or treating at least one condition which benefits from inhibition of at least one aspartyl-protease, comprising administering to a host a composition comprising a therapeutically effective amount of at least one compound of formula (I), 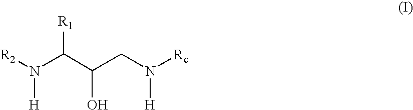
or pharmaceutically acceptable salts thereof, and wherein;
R₁ is selected from 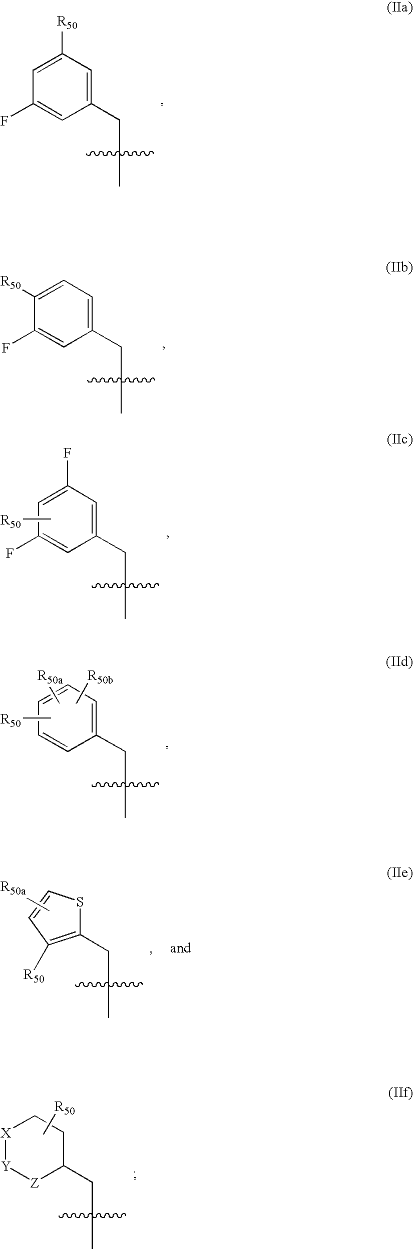
   wherein
X, Y, and Z are independently selected from —C(H)₀₋₂—, —O—, —C(O)—, —NH—, and —N—;
   wherein at least one bond of the (IIf) ring may optionally be a double bond;
R₅₀, R₅₀ₐ, and R_{50b} are independently selected from —H, halogen, —OH, —SH, —CN, —C(O)-alkyl, —NR₇R₈, —NO₂, —S(O)₀₋₂-alkyl, alkyl, alkoxy, —O-benzyl (optionally substituted with at least one group independently selected from —H, —OH, and alkyl), —C(O)—NR₇R₈, alkyloxy, alkoxyalkoxyalkoxy, and cycloalkyl;
   wherein the alkyl, alkoxy, and cycloalkyl groups within R₅₀, R₅₀ₐ, and R_{50b} are optionally substituted with at least one group independently selected from alkyl, halogen, OH, NR₅R₆, CN, haloalkoxy, NR₇R₈, and alkoxy;
R₅ and R₆ are independently selected from —H and alkyl, or
R₅ and R₆, and the nitrogen to which they are attached, form a 5 or 6 membered heterocycloalkyl ring; and
R₇ and R₈ are independently selected from -H, alkyl optionally substituted with at least one group independently selected from —OH, —NH₂, and halogen, -cycloalkyl, and -alkyl-O-alkyl;
R₂ is 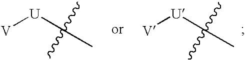
U is selected from —C(O)—, —C(═S)—, —S(O)₀₋₂—, —C(═N—R₂₁)—, —C(═N—OR₂₁)—, —C(O)—NR₂₀—, —C(O)—O—, —S(O)₂—NR₂₀—, and —S(O)₂—O—;
U′ is selected from —C(O)—, —C(═N—R₂₁)—, —C(═N—OR₂₁)—, —C(O)—NR₂₀—, and —C(O)—O—;
V is selected from aryl, heteroaryl, cycloalkyl, heterocycloalkyl, —[C(R₄)(R_{4′})]₁₋₃-D, and -(T)O—,—R_{N};
V′ is selected from -(T)₀₋₁-R_{N′};
   wherein the aryl, heteroaryl, cycloalkyl, and heterocycloalkyl groups included within V and V′ are optionally substituted with at least one independently selected R_{B} group;
   wherein at least one carbon of the aryl, heteroaryl, cycloalkyl, and heterocycloalkyl groups included within V and V′ are optionally replaced with —N—, —O—, —NH—, —C(O)—, —C(S)—, —C(═N—H)—, —C(═N—OH)—, —C(═N-alkyl)-, or —C(═N—O-alkyl)-;
R_{B} at each occurrence is independently selected from halogen, —OH, —CF₃, —OCF₃, —O-aryl, —CN, —NR₁₀₁R′₁₀₁, alkyl, alkoxy, —(CH₂)₀₋₄—(C(O))₀₋₁—(O)₀₋₁-alkyl, —C(O)—OH, —(CH₂)₀₋₃-cycloalkyl, aryl, heteroaryl, and heterocycloalkyl;
   wherein the alkyl, alkoxy, cycloalkyl, aryl, heteroaryl, or heterocycloalkyl groups included within R_{B} are optionally substituted with 1 or 2 groups independently selected from C₁-C₄ alkyl, C₁-C₄ alkoxy, C₁-C₄ haloalkyl, C₁-C₄ haloalkoxy, halogen, —OH, —CN, and —NR₁₀₁R′₁₀₁;
R₁₀₁ and R′₁₀₁ are independently selected from H, alkyl, —(C(O))₀₋₁—(O)₀₋₁-alkyl, —(C(O))₀₋₁—OH, and aryl;
R₄ and R₄ are independently selected from hydrogen, —OH, alkyl, (CH₂)₀₋₃-cycloalkyl, —(CH₂)₀₋₃—OH, fluorine, —CF₃, —OCF₃, —O-aryl, alkoxy, C₃-C₇ cycloalkoxy, aryl, and heteroaryl, or
R₄ and R_{4′} are taken together with the carbon to which they are attached to form a 3, 4, 5, 6, or 7 membered carbocylic ring wherein 1, 2, or 3 carbons of the ring is optionally replaced with —O—, —N(H)—, —N(alkyl)-, —N(aryl)-, —C(O)—, or —S(O)₀₋₂;
D is selected from aryl, heteroaryl, cycloalkyl, and heterocycloalkyl, wherein the aryl, heteroaryl, cycloalkyl, and heterocycloalkyl are optionally substituted with 1 or 2 R_{B} groups;
T is selected from —NR₂₀— and —O—;
R₂₀ is selected from H, —CN, alkyl, haloalkyl, and cycloalkyl;
R₂₁ is selected from H, alkyl, haloalkyl, and cycloalkyl;
R_{N} is selected from —OH, —NH₂, —NH(alkyl), —NH(cycloalkyl), —N(alkyl)(alkyl), —N (alkyl)(cycloalkyl), —N(cycloalkyl)(cycloalkyl), —R′₁₀₀, alkyl-R₁₀₀, —(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, —(CRR′)₁₋₆—NR₁₀₀—R′₁₀₀, —(CRR′)₁₋₆—P(O)(O-alkyl)₂, alkyl-O-allkyl-C(O)OH, and —CH(R_{E1})—(CH₂)₀₋₃-E₁-E₂-E₃;
R_{N′} is —SO₂R′₁₀₀;
R and R′ are independently selected from hydrogen, C₁-C₁₀ alkyl (optionally substituted with at least one group independently selected from OH), C₁-C₁₀ alkylaryl, and C₁-C₁₀ alkylheteroaryl;
R₁₀₀ and R′₁₀₀ are independently selected from
   alkoxy,
   heterocycloalkyl,
   aryl,
   heteroaryl,
   -aryl-W-aryl,
   -aryl-W-heteroaryl,
   -aryl-W-heterocycloalkyl,
   -heteroaryl-W-aryl,
   -heteroaryl-W-heteroaryl,
   -heteroaryl-W-heterocycloalkyl,
   -heterocycloalkyl-W-aryl,
   -heterocycloalkyl-W-heteroaryl,
   -heterocycloalkyl-W-heterocycloalkyl,
   —W—R₁₀₂,
   —CH[(CH₂)₀₋₂—O—R₁₅₀]—(CH₂)₀₋₂-aryl,
   —CH[(CH₂)₀₋₂—O—R₁₅₀]—(CH₂)₀₋₂-heterocycloalkyl,
   —CH[(CH₂)₀₋₂—O—R₁₅₀]—(CH₂)₀₋₂-heteroaryl,
—C₁-C₁₀ alkyl optionally substituted with 1, 2, or 3 R₁₁₅ groups, wherein 1, 2, or 3 carbons of the alkyl group are optionally replaced with a group independently selected from —C(O)— and —NH—,
   -alkyl-O-alkyl optionally substituted with 1, 2, or 3 R₁₁₅ groups,
   -alkyl-S-alkyl optionally substituted with 1, 2, or 3 R₁₁₅ groups, and
   -cycloalkyl optionally substituted with 1, 2, or 3 R₁₁₅ groups;
      wherein the ring portions included within R₁₀₀ and R′₁₀₀ are optionally substituted with 1, 2, or 3 groups independently selected from —OR, —NO₂, halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄-cycloalkyl, —(CH₂)₀₋₄—R₁₁₀, —(CH₂)₀₋₄—R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R₁₃₀, —(CH₂)₀₋₄—C(O)—R₁₄₀, —(CH₂)₀₋₄—C(O)—O——R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—O—P(O)—(O—R₁₁₀)₂, —(CH₂)₀₋₄—O—C(O)—N(R₁₅₀)₂, —(CH₂)₀₋₄—O—CS—N(R₁₅₀)₂, —(CH₂)₀₋₄—O—(R₁₅₀), —(CH₂)₀₋₄—O—R₁₅₀′—C(O)OH, —(CH₂)₀₋₄—S—(R₁₅₀), —(CH₂)₀₋₄—N(R₁₅₀)—SO₂—R₁₀₅, —(CH₂)₀₋₄-cycloalkyl, and (C₁-C₁₀)-alkyl;
R_{E1} is selected from —H, —OH, —NH₂,—NH—(CH₂)₀₋₃-R_{E2}, —NHR_{E8}, —NR_{E350}C(O)R_{E5}, —C₁-C₄ alkyl-NHC(O)R_{E5}, —(CH₂)₀₋₄R_{E8}, —O—(C₁-C₄ alkanoyl), —C₆-C₁₀ aryloxy (optionally substituted with 1, 2, or 3 groups independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂—CF₃, —C≡CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354};
R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10};
R_{E3} and R_{E4} are independently selected from —H, —C₁-C₃ alkyl, and —C₃-C₆ cycloalkyl;
R_{E10} is selected from alkyl, arylalkyl, alkanoyl, and arylalkanoyl;
R_{E5} is selected from cycloalkyl, alkyl (optionally substituted with 1, 2, or 3 groups independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂-C₁-C₄ alkyl, and —C₆-C₁₀ aryloxy), heteroaryl (optionally substituted with 1, 2, or 3 groups independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄haloalkyl), and —NR_{E6}R_{E7};
R_{E6} and R_{E7} are independently selected from —H, alkyl, alkanoyl, aryl, —SO₂—C₁-C₄ alkyl, and aryl-C₁-C₄ alkyl;
R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S-alkyl, and —S—C₂-C₄ alkanoyl;
R_{E9} is selected from H, alkyl, and -aryl C₁-C₄ alkyl;
R_{E350} is selected from H and alkyl;
R_{E351} is selected from aryl-(C₁-C₄ alkyl), alkyl (optionally substituted with 1, 2, or 3 groups independently selected from halogen, cyano, heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl;
   wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy;
R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, NHS(O)₀₋₂-alkyl, —N(alkyl)S(O)₀₋₂-alkyl, —S(O)₀₋₂-heteroaryl, —S(O)₀₋₂-aryl, —NH(arylalkyl), —N(alkyl)(arylalkyl), thioalkoxy, and alkoxy;
   wherein each group included within R₃₅₂ is optionally substituted with 1, 2, 3, 4, or 5 groups independently selected from alkyl, alkoxy, thioalkoxy, halogen, haloalkyl, haloalkoxy, alkanoyl, —NO₂, —CN, alkoxycarbonyl, and aminocarbonyl;
R_{E353} is selected from —O—, —C(O)—, —NH—, —N(alkyl)-, —NH—S(O)₀₋₂—, —N(alkyl)-S(O)₀₋₂—, —S(O)₀₋₂—NH—, —S(O)₀₋₂—N(alkyl)-, —NH—C(S)—, and —N(alkyl)-C(S)—;
R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl;
   wherein each group included within R_{E354} is optionally substituted with 1, 2, 3, 4, or 5 groups independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl;
E₁ is selected from —NR_{E11}— and —C₁-C₆ alkyl—(optionally substituted with 1, 2, or 3 groups selected from C₁-C₄ alkyl),
R_{E11} is selected from —H and alkyl; or
R_{E1} and R_{E11} combine to form —(CH₂)₁₋₄—;
E₂ is selected from a bond, —SO₂—, —SO—, —S—, and —C(O)—; and
E₃ is selected from —H, —C₁-C₄ haloalkyl, —C₅-C₆ heterocycloalkyl (containing at least one group independently selected from N, O, and S,), —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group independently selected from halogen, alkyl, alkoxy, —CN and —NO₂), and arylalkyl (optionally substituted with at least one group independently selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, aryl, —SO₂—C₁-C₄ alkyl, -aryl-C₁-C₄ alkyl, and —C₃-C₈ cycloalkyl C₁-C₄ alkyl;
   or
E₃ₐ, E_{3b}, and the nitrogen to which they are attached form a ring selected from piperazinyl, piperidinyl, morpholinyl, and pyrolidinyl;
   wherein each ring is optionally substituted with 1, 2, 3, or 4 groups independently selected from alkyl, alkoxy, alkoxyalkyl, and halogen;
W is selected from —(CH₂)₀₋₄—, —O—, —S(O)₀₋₂—, —N(R₁₃₅)—, —CR(OH)—, and —C(O)—;
R₁₀₂ and R₁₀₂′ are independently selected from hydrogen and C₁-C₁₀ alkyl (optionally substituted with 1, 2, or 3 groups independently selected from halogen, aryl, and —R₁₁₀);
R₁₀₅ and R′₁₀₅ are independently selected from —H, —R₁₁₀, —R₁₂₀, cycloalkyl, —(C₁-C₂ alkyl)-cycloalkyl, -(alkyl)-O—(C₁-C₃ alkyl), -alkyl optionally substituted with at least one group independently selected from —OH, amine, and halogen; or
R₁₀₅ and R′₁₀₅ together with the atom to which they are attached form a 3, 4, 5, 6 or 7 membered carbocylic ring, wherein one member is optionally a heteroatom selected from —O—, —S(O)₀₋₂—, and —N(R₁₃₅)—, wherein the carbocylic ring is optionally substituted with 1, 2 or 3 R₁₄₀ groups; and wherein the at least one carbon of the carbocylic ring is optionally replaced with —C(O)—;
R₁₁₀ is aryl (optionally substituted with 1 or 2 R₁₂₅ groups);
R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂-(alkyl), alkoxy, and haloalkoxy;
R₁₂₀ is heteroaryl, optionally substituted with 1 or 2 R₁₂₅ groups;
R₁₂₅ at each occurrence is independently selected from halogen, amino, monoalkylamino, dialkylamino, —OH, —CN, —SO₂—NH₂, —SO₂—NH-alkyl, —SO₂—N(alkyl)₂, —SO₂—(C₁-C₄ alkyl), —C(O)—NH₂, —C(O)—NH-alkyl, —C(O)—N(alkyl)₂, alkyl (optionally substituted with 1, 2, or 3 groups independently selected from C₁-C₃ alkyl, halogen, —OH, —SH, —CN, —CF₃, C₁-C₃ alkoxy, amino, monoalkylamino, and dialkylamino), and alkoxy (optionally substituted with 1, 2, or3 halogen);
R₁₃₀ is heterocycloalkyl (optionally substituted with 1 or 2 R₁₂₅ groups);
R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl);
R₁₄₀ at each occurrence is independently selected from heterocycloalkyl (optionally substituted with 1, 2, 3, or 4 groups independently selected from alkyl, alkoxy, halogen, hydroxy, cyano, nitro, amino, monoalkylamino, dialkylamino, haloalkyl, haloalkoxy, amino-alkyl, monoalkylamino-alkyl, dialkylaminoalkyl, and —C(O)H);
R₁₅₀ is independently selected from hydrogen, cycloalkyl, —(C₁-C₂ alkyl)-cycloalkyl, R₁₁₀, R₁₂₀, and alkyl (optionally substituted with 1, 2, 3, or 4 groups independently selected from —OH, —NH₂, C₁-C₃ alkoxy, R₁₁₀, and halogen);
R₁₅₀′ is independently selected from cycloalkyl, —(C₁-C₃ alkyl)-cycloalkyl, R₁₁₀, R₁₂₀, and alkyl (optionally substituted with 1, 2, 3, or 4 groups independently selected from —OH, —NH₂, C₁-C₃ alkoxy, R₁₁₀, and halogen); and
R₁₈₀ is independently selected from morpholinyl, thiomorpholinyl, piperazinyl, piperidinyl, homomorpholinyl, homothiomorpholinyl, homothiomorpholinyl S-oxide, homothiomorpholinyl S,S-dioxide, pyrrolinyl, and pyrrolidinyl;
   wherein each R₁₈₀ is optionally substituted with 1, 2, 3, or 4 groups independently selected from alkyl, alkoxy, halogen, hydroxy, cyano, nitro, amino, monoalkylamino, dialkylamino, haloalkyl, haloalkoxy, aminoalkyl, monoalkylamino-alkyl, and dialkylamino-alkyl, and C(O)H; and wherein the at least one carbon of R₁₈₀ is optionally replaced with —C(O)—;
R_{C} is selected from formulas (IIIa), (IIIb), and (IIIc), 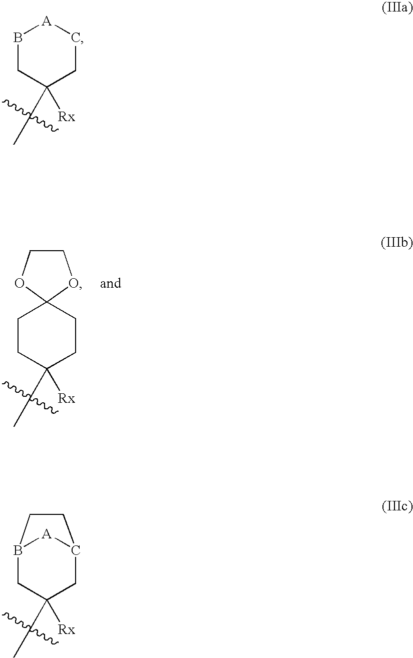
   wherein,
A, B, and C are independently selected from —CH₂—, —O—, —C(O)—, —S(O)₀₋₂—, —NH—, —N(R₂₀₀), —N(CO)₀₋₁R₂₀₀—, and —N(S(O₂)alkyl)-;
   wherein (IIIa), (IIIb), and (IIIc) are each optionally substituted with at least one group independently selected from alkyl, alkoxy, —OH, halogen, —NH₂, —NH(alkyl), —N(alkyl)(alkyl), —NH—C(O)-alkyl, and —NS(O₂)-alkyl;
Rₓ is selected from
   -aryl, heteroaryl, cycloalkyl, heterocycloalkyl, and —Rₓₐ—R_{xb},
   wherein Rₓₐ and R_{xb} are independently selected from -aryl, -heteroaryl, -cycloalkyl, and -heterocycloalkyl;
   wherein each aryl or heteroaryl group within R_{C} is optionally substituted with at least one group independently selected from R₂₀₀;
   wherein each cycloalkyl or heterocycloalkyl within R_{C} is optionally substituted with at least one group independently selected from R₂₁₀; and
   wherein at least one carbon of the heteroaryl or heterocycloalkyl group within R_{C} is independently optionally replaced with a group independently selected from —NH—, —N—, —N(CO)₀₋₁R₂₁₅—, —N(CO)₀₋₁R₂₂₀—, —O—, —C(O)—, —S(O)₀₋₂—, and —NS(O)₀₋₂R₂₀₀;
R₂₀₀ at each occurrence is independently selected from alkyl (optionally substituted with at least one group independently selected from R₂₀₅), —OH, —NO₂, halogen, —CN, —(CH₂)₀₋₄—C(O)H, —(CO)₀₋₁R₂₁₅, —(CO)₀₋₁R₂₂₀, —(CH₂)₀₋₄—C(O)—NR₂₂₀R₂₂₅, —(CH₂)₀₋₄—C(O)—NH(R₂₁₅), —(CH₂)₀₋₄—C(O)-alkyl, —(CH₂)₀₋₄—(CO)₀₋₁-cycloalkyl, —(CH₂)₀₋₄—(CO)₀₋₁-heterocycloalkyl, —(CH₂)₀₋₄—(CO)₀₋₁-aryl, —(CH₂)₀₋₄—(CO)₀₋₁-heteroaryl, —(CH₂)₀₋₄—C(O)—O—R₂₁₅, —(CH₂)₀₋₄—SO₂—NR₂₂₀R₂₂₅, —(CH₂)₀₋₄—S(O)₀₋₂-alkyl, —(CH₂)₀₋₄—S(O)₀₋₂-cycloalkyl, —(CH₂)₀₋₄—N(H or R₂₁₅)—C(O)—O—R₂₁₅, —(CH₂)₀₋₄—N(H or R₂₁₅)—SO₂—R₂₂₀, —(CH₂)₀₋₄—N(H or R₂₁₅)—C(O)—N(R₂₁₅)₂, —(CH₂)₀₋₄—N(H or R₂₁₅)—C(O)—R₂₂₀, —(CH₂)₀₋₄—NR₂₂₀R₂₂₅, —(CH₂)₀₋₄—O—C(O)-alkyl, —(CH₂)₀₋₄—O—(R₂₁₅), —(CH₂)₀₋₄—S—(R₂₁₅), —(CH₂)0-4—C(O)H, —(CH₂)₀₋₄—O-alkyl (optionally substituted with at least one —F), and adamantane;
   wherein each aryl and heteroaryl group included within R₂₀₀ is optionally substituted with at least one group independently selected from R₂₀₅, R₂₁₀, and alkyl (optionally substituted with at least one group independently selected from R₂₀₅ and R₂₁₀);
   wherein each cycloalkyl or heterocycloalkyl group included within R₂₀₀ is optionally substituted with at least one group independently selected from R₂₁₀;
R₂₀₅ at each occurrence is independently selected from alkyl, haloalkoxy, —(CH₂)₀₋₃-cycloalkyl, halogen, —(CH₂)₀₋₆—OH, —O-aryl, —OH, —SH, —(CH₂)₀₋₄—C(O)H, —(CH₂)₀₋₆—CN, —(CH₂)₀₋₆—C(O)—NR₂₃₅R₂₄₀, —(CH₂)₀₋₆—C(O)—R₂₃₅, —(CH₂)₀₋₄—N(H or R₂₁₅)—SO₂—R₂₃₅, —CN, —OCF₃, —CF₃, alkoxy, alkoxycarbonyl, and —NR₂₃₅R₂₄₀;
   R₂₁₀ at each occurrence is independently selected from —OH, —CN, —(CH₂)₀₋₄—C(O)H, alkyl (optionally substituted with at least one group independently selected from R₂₀₅), alkanoyl, —S(O)₂-alkyl, halogen, alkoxy, haloalkoxy, —NR₂₂₀R₂₂₅, cycloalkyl (optionally substituted with at least one group independently selected from R₂₀₅), heterocycloalkyl, heteroaryl, —(CH₂)₀₋₄—NR₂₃₅R₂₄₀, —(CH₂)₀₋₄—NR₂₃₅(alkoxy), —(CH₂)₀₋₄—S—(R₂₁₅), —(CH₂)₀₋₆—OH, —(CH₂)₀₋₆—CN, —(CH₂)₀₋₄—NR₂₃₅—C(O)H, —(CH₂)₀₋₄—NR₂₃₅—C(O)-(alkoxy), —(CH₂)₀₋₄—NR₂₃₅—C(O)—R₂₄₀, —C(O)—NHR₂₁₅, —C(O)-alkyl, —S(O)₂—NR₂₃₅R₂₄₀, —C(O)—N R₂₃₅R₂₄₀, and —S(O)₂-alkyl;
R₂₁₅ at each occurrence is independently selected from alkyl, —(CH₂)₀₋₂-aryl, —(CH₂)₀₋₂-cycloalkyl, —(CH₂)₀₋₂-heteroaryl, —(CH₂)₀₋₂-heterocycloalkyl, and —CO₂—CH₂-aryl;
   wherein the aryl group included within R₂₁₅ is optionally substituted with at least one group independently selected from R₂₀₅ and R₂₁₀, and
   wherein the heterocycloalkyl and heteroaryl groups included within R₂₁₅ are optionally substituted with at least one group independently selected from R₂₁₀;
R₂₂₀ and R₂₂₅ at each occurrence are independently selected from —H, alkyl, —(CH₂)₀₋₄—C(O)H, alkylhydroxyl, alkoxycarbonyl, alkylamino, —S(O)₂-alkyl, alkanoyl (optionally substituted with at least one halogen), —C(O)—NH₂, —C(O)—NH (alkyl), —C(O)—N(alkyl)(alkyl), haloalkyl, —(CH₂)₀₋₂-cycloalkyl, -(alkyl)-O-(alkyl), aryl, heteroaryl, and heterocycloalkyl;
   wherein the aryl, heteroaryl, cycloalkyl and heterocycloalkyl groups included within R₂₂₀ and R₂₂₅ are each optionally substituted with at least one group independently selected from R₂₇₀;
R₂₇₀ at each occurrence is independently selected from —R₂₀₅, alkyl (optionally substituted with at least one group independently selected from R₂₀₅), aryl, halogen, alkoxy, haloalkoxy, —NR₂₃₅R₂₄₀, —OH, —CN, cycloalkyl (optionally substituted with at least one group independently selected from R₂₀₅), —C(O)-alkyl, —S(O)₂—NR₂₃₅R₂₄₀, —C(O)—NR₂₃₅R₂₄₀, —S(O)₂-alkyl, and —(CH₂)₀₋₄—C(O)H;
R₂₃₅ and R₂₄₀ at each occurrence are independently selected from —H, —OH, —CF₃, —OCF₃, —OCH₃, —NHCH₃, —NH(CH₃)₂, —(CH₂)₀₋₄—C(O)(H, or alkyl), alkyl, alkanoyl, —SO₂-alkyl, and aryl.

In an embodiment, the hydroxyl alpha to the —(CHR₁)— group of compounds of formula (1) may be optionally replaced by —NH₂, —NH(R₇₀₀), —N (R₇₀₀)(R₇₀₀), —SH, and —SR₇₀₀, wherein —R₇₀₀ is alkyl (optionally substituted with at least one group independently selected from R₁₁₀, R₁₁₅, R₂₀₅, and R₂₁₀).

In another embodiment U is selected from —C(O)—, —C(S)—, —S(O)₀₋₂—, —C(═NR₂₁)—, —C(═N—OR₂₁)—, —C(O)—NR₂₀—, —C(O)—O—, —S(O)₂—NR₂₀—, and —S(O)₂; —O—; and V is -(T)₀₋₁-R_{N}.

In another embodiment U′ is selected from —C(O)—, —C(═NR₂₁)—, —C(═N—OR₂₁)—, —C(O)—NR₂₀—, —C(O)—O—, —S(O)₂—NR₂₀—, and —S(O)₂—O—; and V′ is -(T)₀₋₁-R_{N′}.

In another embodiment U is selected from —S(O)₂—NR₂₀— and —S(O)₂—O—.

In another embodiment U is selected from —C(O)—NR₂₀— and —C(O)—O—.

In another embodiment R_{N} is 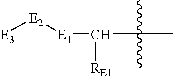
wherein
E₁ is selected from —NR_{E11}— and —C₁-C₆ alkyl—(optionally substituted with 1, 2, or 3 groups selected from C₁-C₄ alkyl); R_{E1} is —NH₂ and R_{E11} is selected from —H and alkyl; or R_{E1} and R_{E11} combine to form —(CH₂)₁₋₄—;
E₂ is selected from a bond; —SO₂, —SO, —S, and —C(O);
E₃ is selected from —H, —C₁-C₄ haloalkyl, —C₅-C₆ heterocycloalkyl (containing at least one group independently selected from N, O, and S), —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl, and halogen), alkoxy, aryl (optionally substituted with at least one group independently selected from halogen, —C₁-C₄ alkyl, —C₁-C₄ alkoxy, —CN, and —NO₂), and aryl C₁-C₄ alkyl (optionally substituted with at least one group independently selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkanoyl, aryl, —SO₂—C₁-C₄ alkyl, -aryl C₁-C₄ alkyl, and —C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrolidinyl, wherein each ring is optionally substituted with 1, 2, 3, or 4 groups independently selected from alkyl, alkoxy, alkoxyalkyl, and halogen.

In another embodiment R_{N} is selected from alkyl, —(CH₂)₀₋₂-aryl, —C₂-C₆ alkyl, —C₂-C₆ alkyl, —C₃-C₇ cycloalkyl, and —(CH₂)₀₋₂-heteroaryl.

In another embodiment U is selected from —N(R₂₀)—C(O)— and —O—C(O)—.

In another embodiment U is —C(O)— and T is —N(R₂₀)— or —O—.

In another embodiment U is —C(O)— and T is —O—.

In another embodiment U is —C(O)— and T is —NH—.

In another embodiment U is —SO₂— and V is -T₀₋₁-R_{N}.

In another embodiment U is selected from —C(O)—, and —S(O)₀₋₂—; and V is —[C(R₄)(R_{4′})]₁₋₃-D.

In another embodiment V is selected from —(CH₂)₁₋₃-aryl and -(CH₂)₁₋₃-heteroaryl, wherein each ring is independently optionally substituted with 1 or 2 groups independently selected from halogen, —OH, —OCF₃, —O—-phenyl, —CN, —NR₁₀₁R′₁₀₁, alkyl, alkoxy, —(CH₂)₀₋₃(C₃-C₇cycloalkyl), aryl, heteroaryl, and heterocycloalkyl,
wherein the alkyl, alkoxy, cycloalkyl, aryl, heteroaryl, or heterocycloalkyl groups are optionally substituted with 1 or 2 groups independently selected from —C₁-C₄ alkyl, —C₁-C₄ alkoxy, —C₁-C₄ haloalkyl, —C₁-C₄ haloalkoxy, halogen, —OH, —CN, and —NR₁₀₁R′₁₀₁.

In another embodiment U is —C(O)—.

In another embodiment U is selected from —C(O)— and —S(O)₀₋₂—; and V is selected from aryl, heteroaryl, cycloalkyl, and heterocycloalkyl; wherein the aryl, heteroaryl, cycloalkyl, and heterocycloalkyl groups included within V are optionally substituted with at least one independently selected R_{B} group.

In another embodiment V is selected from aryl and heteroaryl, wherein each ring is independently optionally substituted with 1 or 2 groups independently selected from halogen, —OH, —OCF₃, —O-phenyl, —CN, —NR₁₀₁R′₁₀₁, alkyl, alkoxy, —(CH₂)₀₋₃(C₃-C₇ cycloalkyl), aryl, heteroaryl, and heterocycloalkyl, wherein the alkyl, alkoxy, cycloalkyl, aryl, heteroaryl, or heterocycloalkyl groups are optionally substituted with 1 or 2 groups independently selected from —C₁-C₄ alkyl, —C₁-C₄ alkoxy, —C₁-C₄ haloalkyl, —C₁-C₄ haloalkoxy, halogen, —OH, —CN, and —NR₁₀₁R′₁₀₁.

In another embodiment, R₁ is selected from a —CH₂-phenyl, wherein the phenyl ring is optionally substituted with at least one group independently selected from halogen, —C₁-C₂ alkyl, —C1-C₂ alkoxy, and —OH. In various other embodiments, R₁ is selected from 3-Allyloxy-5-fluoro-benzyl, 3-Benzyloxy-5-fluoro-benzyl, 4-hydroxy-benzyl, 3-hydroxy-benzyl, 3-propyl-thiophen-2-yl-methyl, 3,5-difluoro-2-propylamino-benzyl, 5-chloro-thiophen-2-yl-methyl, 5-chloro-3-ethyl-thiophen-2-yl-methyl, 3,5-difluoro-2-hydroxy-benzyl, 2-ethylamino-3,5-difluoro-benzyl, piperidin-4-yl-methyl, 2-oxo-piperidin-4-yl-methyl, 2-oxo-1,2-dihydro-pyridin-4-yl-methyl, 5-hydroxy-6-oxo-6H-pyran-2-yl-methyl, 2-Hydroxy-5-methyl-benzamide, 3,5-Difluoro-4-hydroxy-benzyl, 3,5-Difluoro-benzyl, 3-Fluoro-4-hydroxy-benzyl, 3-Fluoro-5-[2-(2-methoxy-ethoxy)-ethoxy]-benzyl, 3-Fluoro-5-heptyloxy-benzyl, 3-Fluoro-5-hexyloxy-benzyl, 3-Fluoro-5-hydroxy-benzyl, 3-Fluoro-benzyl, and the like.

In another embodiment, R₂ is selected from glyoxylic acid, crotonic acid, pyruvic acid, butyric acid, sarcosine, 3-hydroxy-propionic acid, methoxyacetic acid, chloroacetic acid, penta-2,4-dienoic acid, pent-4-ynoic acid, 1-methyl-cyclopropanecarboxylic acid, pent-4-enoic acid, cyclopropylacetic acid, cyclobutanecarboxylic acid, trans-2-pentenoic acid, valeric acid, DL-2-ethylpropionic acid, isovaleric acid, 2-hydroxy-2-methyl-propionic acid, ethoxyacetic acid, DL-2-hydroxy-n-butyric acid, furan-3-carboxylic acid, 1H-pyrazole-4-carboxylic acid, 1H-imidazole-4-carboxylic acid, cyclopent-1-enecarboxylic acid, 4-Methyl-pent-2-enoic acid, cyclopentanecarboxylic acid, trans-2-hexenoic acid, 2-oxo-pentanoic acid, levulinic acid, tetrahydro-3-fluroic acid, tetrahydrofuran-2-carboxylic acid, caproic acid, tert-butylacetic acid, methylmalonic acid, 2-hydroxy-3-methyl-butyric acid, benzoic acid, 2-chloro-butyric acid, picolonic acid, nicotinic acid, isonicotinic acid, pyrazine-2-carboxylic acid, 3-methyl-furan-2-carboxylic acid, 1-methyl-1H-pyrazole-3-carboxylic acid, cyclopent-2-enyl-acetic acid, 5-methyl-isoxazole-3-carboxylic acid, thiophene-3-carboxylic acid, 2-Methyl-hex-2-enoic acid, L-pyroglutamic acid, 5-oxo-pyrrolidine-2-carboxylic acid, D-pyroglutamic acid, N-methylaleamic acid, thiazole 5-carboxylic acid, N-Me-Pro-OH, 3-Methyl-pyrrolidine-2-carboxylic acid, itaconic acid, citraconic acid, 2-oxo-imidazolidine-4-carboxylic acid, 4-Methyl-2-oxo-pentanoic acid, enanthic acid, L-hydroxyproline, cis-4-hydroxy-D-proline, 6-Amino-hexanoic acid, oxalacetic acid, Mono-methyl succinate, Butoxy-acetic acid, (S)-(−)-2-hydroxy-3,3-dimethylbutyric acid, (2-methoxy-ethoxy)-acetic acid, Phenylacetic acid, 5-Chloro-pentanoic acid, Anthranilic acid, Aminonicotinic acid, 3-Hydroxy-pyridine-2-carboxylic acid, 2-Hydroxy-nicotinic acid, Furan-2-yl-oxo-acetic acid, 5-Formyl-furan-2-carboxylic acid, 6-Hydroxy-pyrimidine-4-carboxylic acid, 3-Furan-2-yl-propionic acid, norbornane-2-carboxylic acid, 1-cyclohexenylacetic acid, 3,5-Dimethyl-isoxazole-4-carboxylic acid, Hexa-2,4-dienedioic acid, (2-Oxo-cyclopentyl)-acetic acid, 5-Methyl-thiophene-2-carboxylic acid, Thiophene-2-acetic acid, cylcohexylacetic acid, methyl cyclohexanone-2-carboxylate, (2-Imino-imidazolidin-1-yl)-acetic acid, 4-amino-cyclohexanecarboxylic acid, 2-methylene-succinic acid 1-methyl ester, Trans-beta-hydromuconic acid, Octanoic acid, 2-Propyl-pentanoic acid, 4-Acetylamino-butyric acid, 2-Oxo-pentanedioic acid, N-carbamyl-alpha-aminoisobutyric acid, 4-cyano-benzoic acid, 2-Acetylamino-3-hydroxy-propionic acid, and the like.

In another embodiment, R_{C} is selected from 4-(3-Ethyl-phenyl)-tetrahydro-pyran, 1-Cyclohexyl-3-ethyl-benzene, 1-Cyclohexyl-3-isobutyl-benzene, 1-Cyclohexyl-3-isopropyl-benzene, 1-Cyclohexyl-3-(2,2-dimethyl-propyl)-benzene, 1-tert-Butyl-3-cyclohexyl-benzene, 1-Cyclohexyl-3-ethynyl-benzene, 8-(3-Isopropyl-phenyl)-1 ,4-dioxa-spiro[4.5]decane, 4-(3-Isopropyl-phenyl)-cyclohexanone, 2-(3-Cyclohexyl-phenyl)-4-methyl-thiophene, 1-[5-(3-Cyclohexyl-phenyl)-thiophen-2-yl]-ethanone, 3-(3-Cyclohexyl-phenyl)-furan, 3-(3-Cyclohexyl-phenyl)-thiophene, 5-(3-Cyclohexyl-phenyl)-thiophene-2-carbaldehyde, 2-(3-Cyclohexyl-phenyl)-furan-3-carbaldehyde, N-(3′-Cyclohexyl-biphenyl-3-yl)-acetamide, 4-(3-tert-Butyl-phenyl)-tetrahydro-pyran, 1-Cyclohexyl-3-trifluoromethyl-benzene, 1-sec-Butyl-3-cyclohexyl-benzene, 1-cyclohexyl-3-pentyl-benzene, 1-Cyclohexyl-3-(3-methyl-butyl)-benzene, 1-Cyclohexyl-3-(1-ethyl-propyl)-benzene, 1-Cyclohexyl-3-cyclopentyl-benzene, 1-Cyclohexyl-3-pent-4-enyl-benzene, 3-(3-Cyclohexyl-phenyl)-propionic acid ethyl ester, 2-(3-Cyclohexyl-phenyl)-pyridine, 2-(3-Cyclohexyl-phenyl)-3-methyl-pyridine, 2-(3-Cyclohexyl-phenyl)-thiazole, 2-(3-Cyclohexyl-phenyl)-3-methyl-thiophene, 1-Cyclohexyl-3-(2-fluoro-benzyl)-phenylene, 1-Cyclohexyl-3-(4-fluoro-benzyl)-phenylene, 2-(3-Cyclohexyl-phenyl)-adamantane, 4-(3-Isopropyl-phenyl)-tetrahydro-thiopyran, 4-(3-Isopropyl-phenyl)-tetrahydro-thiopyran 1,1-dioxide, 1-[4-(3-Isopropyl-phenyl)-piperidin-1-yl]-ethanone, 4-(3-Isopropyl-phenyl)-1-methanesulfonyl-piperidine, 4-(3-Isopropyl-phenyl)-tetrahydro-thiopyran 1-oxide, 2,2,2-Trifluoro-1-[4-(3-isopropyl-phenyl)-piperidin-1-yl]-ethanone, 4-(3-Isopropyl-phenyl)-piperidine-1-carbaldehyde, 1-Cyclohexyl-3-cyclopropyl-benzene, 1-Bromo-3-tert-butyl-5-cyclohexyl-benzene, 4-(3-tert-Butyl-phenyl)-1-methanesulfonyl-piperidine, 4-(3-tert-Butyl-phenyl)-1-ethanesulfonyl-piperidine, 3-Bromo-5-(3-cyclohexyl-phenyl)-[1,2,4]thiadiazole, 2-(3-Cyclohexyl-phenyl)-1-methyl-1H-imidazole, 4-(3-Cyclohexyl-phenyl)-3,5-dimethyl-3H-pyrazole, 3-(3-Cyclohexyl-phenyl)-2,5-dimethyl-pyrazine, 3-(3-Cyclohexyl-phenyl)-pyrazine-2-carbonitrile, 4-(3-Cyclohexyl-phenyl)-thiazole, 2-(3-Cyclohexyl-phenyl)-isonicotinonitrile, 2-(3-Cyclohexyl-phenyl)-pyrazine, 3-(3-Cyclohexyl-phenyl)-6-methyl-pyridazine, 3-(3-Cyclohexyl-phenyl)-thiophene-2-carbonitrile, 2-Chloro-3-(3-cyclohexyl-phenyl)-thiophene, 1-[4-(3-Cyclohexyl-phenyl)-thiophen-2-yl]-ethanone, 3-Cyclohexyl-benzonitrile, and the like.

In another embodiment, Rₓ is selected from 3-(1,1-dimethyl-propyl)-phenyl, 3-(1-ethyl-propyl)-phenyl, 3-(1H-pyrrol-2-yl)-phenyl, 3-(1-hydroxy-1-methyl-ethyl)-phenyl, 3-(1-methyl-1H-imidazol-2-yl)-phenyl, 3-(1-methyl-cyclopropyl)-phenyl, 3-(2,2-dimethyl-propyl)-phenyl, 3-(2,5-dihydro-1H-pyrrol-2-yl)-phenyl, 3-(2-Chloro-thiophen-3-yl)-phenyl, 3-(2-Cyano-thiophen-3-yl)-phenyl, 3-(2-fluoro-benzyl)-phenyl, 3-(3,5-dimethyl-3H-pyrazol-4-yl)-phenyl, 3-(3,6-dimethyl-pyrazin-2-yl)-phenyl, 3-(3-Cyano-pyrazin-2-yl)-phenyl, 3-(3-formyl-furan-2-yl)-phenyl, 3-(3H-[1,2,3]triazol-4-yl)-phenyl, 3-(3H-imidazol-4-yl)-phenyl, 3-(3-methyl-butyl)-phenyl, 3-(3-methyl-pyridin-2-yl)-phenyl, 3-(3-methyl-thiophen-2-yl)-phenyl, 3-(4-Cyano-pyridin-2-yl)-phenyl, 3-(4-fluoro-benzyl)-phenyl, 3-(4H-[1,2,4]triazol-3-yl)-phenyl, 3-(4-methyl-thiophen-2-yl)-phenyl, 3-(5-Acetyl-thiophen-2-yl)-phenyl, 3-(5-Acetyl-thiophen-3-yl)-phenyl, 3-(5-formyl-thiophen-2-yl)-phenyl, 3-(5-oxo-pyrrolidin-2-yl)-phenyl, 3-(6-methyl-pyridazin-3-yl)-phenyl, 3-(6-methyl-pyridin-2-yl)-phenyl, 3-(Cyano-dimethyl-methyl)-phenyl, 3-[1-(2-tert-Butyl-pyrimidin-4-yl)-cyclohexylamino, 3-[1,2,3]triazol-1-yl-phenyl, 3-[1,2,4]oxadiazol-3-yl-phenyl, 3-[1,2,4]oxadiazol-5-yl-phenyl, 3-[1,2,4]thiadiazol-3-yl-phenyl, 3-[1,2,4]thiadiazol-5-yl-phenyl, 3-[1,2,4]triazol-4-yl-phenyl, 3-Acetyl-5-tert-butyl-phenyl, 3′-Acetylamino-biphenyl-3-yl, 3-Adamantan-2-yl-phenyl, 3-Bromo-[1,2,4]thiadiazol-5-yl)-phenyl, 3-Bromo-5-tert-butyl-phenyl, 3-Cyano-phenyl, 3-Cyclobutyl-phenyl, 3-Cyclopentyl-phenyl, 3-Cyclopropyl-phenyl, 3-ethyl-phenyl, 3-ethynyl-phenyl, 3-fluoro-5-(2-hydroxy-1,1-dimethyl-ethyl)-phenyl, 3-furan-3-yl-phenyl, 3-imidazol-1-yl-phenyl, 3-isobutyl-phenyl, 3-isopropyl-phenyl, 3-isoxazol-3-yl-phenyl, 3-isoxazol-4-yl-phenyl, 3-isoxazol-5-yl-phenyl, 3-pent-4-enyl-phenyl, 3-pentyl-phenyl, 3-Phenyl-propionic acid ethyl ester, 3-pyrazin-2-yl-phenyl, 3-pyridin-2-yl-phenyl, 3-pyrrolidin-2-yl-phenyl, 3-sec-Butyl-phenyl, 3-tert-Butyl-4-chloro-phenyl, 3-tert-Butyl-4-cyano-phenyl, 3-tert-Butyl-4-ethyl-phenyl, 3-tert-Butyl-4-methyl-phenyl, 3-tert-Butyl-4-trifluoromethyl-phenyl, 3-tert-Butyl-5-chloro-phenyl, 3-tert-Butyl-5-cyano-phenyl, 3-tert-Butyl-5-ethyl-phenyl, 3-tert-Butyl-5-fluoro-phenyl, 3-tert-Butyl-5-methyl-phenyl, 3-tert-Butyl-5-trifluoromethyl-phenyl, 3-tert-Butyl-phenyl, 3-thiazol-2-yl-phenyl, 3-thiazol-4-yl-phenyl, 3-thiophen-3-yl-phenyl, 3-trifluoromethyl-phenyl, 4-Acetyl-3-tert-butyl-phenyl, 4-tert-Butyl-pyridin-2-yl, 4-tert-Butyl-pyrimidin-2-yl, 5-tert-Butyl-pyridazin-3-yl, and 6-tert-Butyl-pyridazin-4-yl, 6-tert-Butyl-pyrimidin-4-yl, and the like.

Among the compounds of formula (I), or pharmaceutically acceptable salts thereof, examples include 3-Amino-N-[3-[4-(3-tert-butyl-phenyl)-tetrahydro-pyran-4-ylamino]-1-(3,5-difluoro-benzyl)-2-hydroxy-propyl]-butyramide, [3-[1-(3-tert-Butyl-phenyl)-cyclohexylamino]-1-(3,5-difluoro-benzyl)-2-hydroxy-propyl]-carbamic acid cyclopropyl ester, Cyclobutanecarboxylic acid [3-[1-(3-tert-butyl-phenyl)-cyclohexylamino]-1-(3,5-difluoro-benzyl)-2-hydroxy-propyl]-amide, Furan-2-carboxylic acid [3-[4-(3-tert-butyl-phenyl)-tetrahydro-pyran-4-ylamino]-1-(3,5-difluoro-benzyl)-2-hydroxy-propyl]-amide, Cyclopent-1-enecarboxylic acid [3-[1-(3-tert-butyl-phenyl)-cyclohexylamino]-1-(3,5-difluoro-benzyl)-2-hydroxy-propyl]-amide, 1-[3-[1-(3-tert-Butyl-phenyl)-cyclohexylamino]-1-(3,5-difluoro-benzyl)-2-hydroxy-propyl]-3-cyclopropyl-urea, N-[3-[1-(3-tert-Butyl-phenyl)-cyclohexylamino]-1-(3,5-difluoro-benzyl)-2-hydroxy-propyl]-2-phenyl-acetamide, 4-[3-[1-(3-tert-Butyl-phenyl)-cyclohexylamino]-1-(3,5-difluoro-benzyl)-2-hydroxy-propylcarbamoyl]-but-2-enoic acid, 5-Acetylamino-pentanoic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-2-(2-imino-imidazolidin-1-yl)-acetamide, 3-Methyl-isoxazole-5-carboxylic acid [3-[4-(3-tert-butyl-phenyl)-tetrahydro-pyran-4-ylamino]-1-(3,5-difluoro-benzyl)-2-hydroxy-propyl]-amide, Cyclopropanecarbothioic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-benzene sulfonamide, 2-Butoxy-N-[3-[1-(3-tert-butyl-phenyl)-cyclohexylamino]-1-(3,5-difluoro-benzyl)-2-hydroxy-propyl]-acetamide, 1-[3-[1-(3-tert-Butyl-phenyl)-cyclohexylamino]-1-(3,5-difluoro-benzyl)-2-hydroxy-propyl]-3-phenyl-urea, [3-[l-(3-tert-Butyl-phenyl)-cyclohexylamino]-1-(3,5-difluoro-benzyl)-2-hydroxy-propyl]-carbamic acid phenyl ester, 6-Chloro-hexanoic acid [3-[1-(3-tert-butyl-phenyl)-cyclohexylamino]-1-(3,5-difluoro-benzyl)-2-hydroxy-propyl]-amide, 2-Oxo-imidazolidine-4-carboxylic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-2-(2-oxo-cyclopentyl)-acetamide, N-[3-[1-(3-tert-Butyl-phenyl)-cyclohexylamino]-1-(3,5-difluoro-benzyl)-2-hydroxy-propyl]-3,3-dimethyl-butyramide, 4-[3-[1-(3-tert-Butyl-phenyl)-cyclohexylamino]-1-(3,5-difluoro-benzyl)-2-hydroxy-propylcarbamoyl]-butyric acid isopropyl ester, Hexanedioic acid amide [3-[1-(3-tert-butyl-phenyl)-cyclohexylamino]-1-(3,5-difluoro-benzyl)-2-hydroxy-propyl]-amide, 3-Acetylamino-N-[3-[1-(3-tert-butyl-phenyl)-cyclohexylamino]-1-(3,5-difluoro-benzyl)-2-hydroxy-propyl]-propionamide, 5-Acetylamino-pentanoic acid [3-[1-(3-tert-butyl-phenyl)-cyclohexylamino]-1-(3,5-difluoro-benzyl)-2-hydroxy-propyl]-amide, 1-[3-[1-(3-tert-Butyl-phenyl)-cyclohexylamino]-1-(3,5-difluoro-benzyl)-2-hydroxy-propyl]-3-propyl-urea, 1-tert-Butyl-3-[3-[1-(3-tert-butyl-phenyl)-cyclohexylamino]-1-(3,5-difluoro-benzyl)-2-hydroxy-propyl]-urea, 1-[3-[1-(3-tert-Butyl-phenyl)-cyclohexylamino]-1-(3,5-difluoro-benzyl)-2-hydroxy-propyl]-3-isopropyl-urea, 1-[3-[1-(3-tert-Butyl-phenyl)-cyclohexylamino]-1-(3,5-difluoro-benzyl)-2-hydroxy-propyl]-3-(3,5-dimethyl-isoxazol-4-yl)-urea, Pentanedioic acid amide [3-[1-(3-tert-butyl-phenyl)-cyclohexylamino]-1-(3,5-difluoro-benzyl)-2-hydroxy-propyl]-amide, 1-[3-[1-(3-tert-Butyl-phenyl)-cyclohexylamino]-1-(3,5-difluoro-benzyl)-2-hydroxy-propyl]-3-ethyl-urea, 3-{3-[3-[1-(3-tert-Butyl-phenyl)-cyclohexylamino]-1-(3,5-difluoro-benzyl)-2-hydroxy-propyl]-ureido}-propionic acid ethyl ester, 2-{3-[3-[1-(3-tert-Butyl-phenyl)-cyclohexylamino]-1-(3,5-difluoro-benzyl)-2-hydroxy-propyl]-ureido}-3-methyl-butyric acid ethyl ester, 2-{3-[3-[1-(3-tert-Butyl-phenyl)-cyclohexylamino]-l-(3,5-difluoro-benzyl)-2-hydroxy-propyl]-ureido}-4-methyl-pentanoic acid ethyl ester, 6-[3-[1-(3-tert-Butyl-phenyl)-cyclohexylamino]-1-(3,5-difluoro-benzyl)-2-hydroxy-propylcarbamoyl]-hexanoic acid methyl ester, Pentanedioic acid [3-[1-(3-tert-butyl-phenyl)-cyclohexylamino]-1-(3,5-difluoro-benzyl)-2-hydroxy-propyl]-amide methylamide, 4-[3-[1-(3-tert-Butyl-phenyl)-cyclohexylamino]-1-(3,5-difluoro-benzyl)-2-hydroxy-propylcarbamoyl]-butyric acid methyl ester, 4-[3-[1-(3-tert-Butyl-phenyl)-cyclohexylamino]-1-(3,5-difluoro-benzyl)-2-hydroxy-propylcarbamoyl]-butyric acid, 5-[3-[1-(3-tert-Butyl-phenyl)-cyclohexylamino]-1-(3,5-difluoro-benzyl)-2-hydroxy-propylcarbamoyl]-pentanoic acid methyl ester, 5-[3-[1-(3-tert-Butyl-phenyl)-cyclohexylamino]-1-(3,5-difluoro-benzyl)-2-hydroxy-propylcarbamoyl]-pentanoic acid, N-[3-[1-(3-tert-Butyl-phenyl)-cyclohexylamino]-1-(3,5-difluoro-benzyl)-2-hydroxy-propyl]-2-(1H-indol-3-yl)-acetamide, N-[3-[1-(3-tert-Butyl-phenyl)-cyclohexylamino]-1-(3,5-difluoro-benzyl)-2-hydroxy-propyl]-4-sulfamoyl-butyramide, N-[3-[1-(3-tert-Butyl-phenyl)-cyclohexylamino]-1-(3,5-difluoro-benzyl)-2-hydroxy-propyl]-3-methyl-butyramide, N-[3-[1-(3-tert-Butyl-phenyl)-cyclohexylamino]-1-(3,5-difluoro-benzyl)-2-hydroxy-propyl]-2-methyl-butyramide, N-[3-[1-(3-tert-Butyl-phenyl)-cyclohexylamino]-1-(3,5-difluoro-benzyl)-2-hydroxy-propyl]-2,2-dimethyl-propionamide, But-2-enoic acid [3-[1-(3-tert-butyl-phenyl)-cyclohexylamino]-1-(3,5-difluoro-benzyl)-2-hydroxy-propyl]-amide, Pent-4-enoic acid [3-[1-(3-tert-butyl-phenyl)-cyclohexylamino]-1-(3,5-difluoro-benzyl)-2-hydroxy-propyl]-amide, Hex-3-enoic acid [3-[1-(3-tert-butyl-phenyl)-cyclohexylamino]-1-(3,5-difluoro-benzyl)-2-hydroxy-propyl]-amide, 4-Methyl-pent-2-enoic acid [3-[1-(3-tert-butyl-phenyl)-cyclohexylamino]-1-(3,5-difluoro-benzyl)-2-hydroxy-propyl]-amide, Pent-4-ynoic acid [3-[1-(3-tert-butyl-phenyl)-cyclohexylamino]-1-(3,5-difluoro-benzyl)-2-hydroxy-propyl]-amide, 1-Methyl-cyclopropanecarboxylic acid [3-[1-(3-tert-butyl-phenyl)-cyclohexylamino]-1-(3,5-difluoro-benzyl)-2-hydroxy-propyl]-amide, Cyclopentanecarboxylic acid [3-[1-(3-tert-butyl-phenyl)-cyclohexylamino]-1-(3,5-difluoro-benzyl)-2-hydroxy-propyl]-amide, Furan-3-carboxylic acid [3-[1-(3-tert-butyl-phenyl)-cyclohexylamino]-1-(3,5-difluoro-benzyl)-2-hydroxy-propyl]-amide, Tetrahydro-furan-2-carboxylic acid [3-[1-(3-tert-butyl-phenyl)-cyclohexylamino]-1-(3,5-difluoro-benzyl)-2-hydroxy-propyl]-amide, Tetrahydro-furan-3-carboxylic acid [3-[1-(3-tert-butyl-phenyl)-cyclohexylamino]-1-(3,5-difluoro-benzyl)-2-hydroxy-propyl]-amide, 1H-Imidazole-4-carboxylic acid [3-[1-(3-tert-butyl-phenyl)-cyclohexylamino]-1-(3,5-difluoro-benzyl)-2-hydroxy-propyl]-amide, 5-Methyl-isoxazole-3-carboxylic acid [3-[1-(3-tert-butyl-phenyl)-cyclohexylamino]-1-(3,5-difluoro-benzyl)-2-hydroxy-propyl]-amide, 5-Oxo-pyrrolidine-2-carboxylic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-benzamide, Pyridine-2-carboxylic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-nicotinamide, N-[3-[1-(3-tert-Butyl-phenyl)-cyclohexylamino]-1-(3,5-difluoro-benzyl)-2-hydroxy-propyl]-isonicotinamide, Pyrazine-2-carboxylic acid [3-[1-(3-tert-butyl-phenyl)-cyclohexylamino]-1-(3,5-difluoro-benzyl)-2-hydroxy-propyl]-amide, tert-butyl (4-(1-(3-tert-butylphenyl)cyclohexylamino)-1-(3,5-difluorophenyl)-3-hydroxybutan-2-ylcarbamoyl)methylcarbamate, tert-butyl 4-(1-(3-(2-cyanopropan-2-yl)phenyl)cyclohexylamino)-1-(3,5-difluorophenyl)-3-hydroxybutan-2-ylcarbamate, N-[3-[1-(3-tert-Butyl-phenyl)-cyclohexylamino]-1-(3,5-difluoro-benzyl)-2-hydroxy-propyl]-propionamide, N-[3-[1-(3-tert-Butyl-phenyl)-cyclohexylamino]-1-(3,5-difluoro-benzyl)-2-hydroxy-propyl]-butyramide, N-[3-[1-(3-tert-Butyl-phenyl)-cyclohexylamino]-1-(3,5-difluoro-benzyl)-2-hydroxy-propyl]-2-cyclopropyl-acetamide, Pentanoic acid [3-[1-(3-tert-butyl-phenyl)-cyclohexylamino]-1-(3,5-difluoro-benzyl)-2-hydroxy-propyl]-amide, Pent-2-enoic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-2-ethoxy-acetamide, N-[3-[1-(3-tert-Butyl-phenyl)-cyclohexylamino]-1-(3,5-difluoro-benzyl)-2-hydroxy-propyl]-2-methoxy-acetamide, Thiophene-2-carboxylic acid [3-[1-(3-tert-butyl-phenyl)-cyclohexylamino]-1-(3,5-difluoro-benzyl)-2-hydroxy-propyl]-amide, Thiophene-3-carboxylic acid [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-2-pyridin-3-yl-acetamide, N-[3-[1-(3-tert-Butyl-phenyl)-cyclohexylamino]-1-(3,5-difluoro-benzyl)-2-hydroxy-propyl]-2-pyridin-4-yl-acetamide, 4,7,7-Trimethyl-3-oxo-2-oxa-bicyclo[2.2.1)heptane-1-carboxylic acid [3-[1-(3-tert-butyl-phenyl)-cyclohexylamino]-1-(3,5-difluoro-benzyl)-2-hydroxy-propyl]-amide, Heptanedioic acid amide [3-[1-(3-tert-butyl-phenyl)-cyclohexylamino]-1-(3,5-difluoro-benzyl)-2-hydroxy-propyl]-amide, N-[3-[1-(3-tert-Butyl-phenyl)-cyclohexylamino]-1-(3,5-difluoro-benzyl)-2-hydroxy-propyl]-2,2-difluoro-acetamide, N-[3-[1-(3-tert-Butyl-phenyl)-cyclohexylamino]-1-(3,5-difluoro-benzyl)-2-hydroxy-propyl]-2-tetrazol-1-yl-acetamide, {[3-[1-(3-tert-Butyl-phenyl)-cyclohexylamino]-1-(3,5-difluoro-benzyl)-2-hydroxy-propylcarbamoyl]-methyl}-phosphonic acid diethyl ester, N-[3-[1-(3-tert-Butyl-phenyl)-cyclohexylamino]-1-(3,5-difluoro-benzyl)-2-hydroxy-propyl]-2-(2,5-dioxo-imidazolidin-4-yl)-acetamide, N-[3-[1-(3-tert-Butyl-phenyl)-cyclohexylamino]-1-(3,5-difluoro-benzyl)-2-hydroxy-propyl]-2-(2-methoxy-ethoxy)-acetamide, N-[3-[1-(3-tert-Butyl-phenyl)-cyclohexylamino]-1-(3,5-difluoro-benzyl)-2-hydroxy-propyl]-2-(5-methyl-isoxazol-3-yl)-acetamide, N-[3-[1-(3-tert-Butyl-phenyl)-cyclohexylamino]-1-(3,5-difluoro-benzyl)-2-hydroxy-propyl]-2-thiophen-2-yl-acetamide, N-[3-[1-(3-tert-Butyl-phenyl)-cyclohexylamino]-1-(3,5-difluoro-benzyl)-2-hydroxy-propyl]-2-hydroxy-2-phenyl-acetamide, N-[3-[1-(3-tert-Butyl-phenyl)-cyclohexylamino]-1-(3,5-difluoro-benzyl)-2-hydroxy-propyl]-2-hydroxy-propionamide, N-[3-[1-(3-tert-Butyl-phenyl)-cyclohexylamino]-1-(3,5-difluoro-benzyl)-2-hydroxy-propyl]-3-methoxy-propionamide, N-[3-[1-(3-tert-Butyl-phenyl)-cyclohexylamino]-1-(3,5-difluoro-benzyl)-2-hydroxy-propyl]-N′, N′-dimethyl-succinamide, methyl 3-(4-(1-(3-tert-butylphenyl)cyclohexylamino)-1-(3,5-difluorophenyl)-3-hydroxybutan-2-ylcarbamoyl)propanoate, N-(4-(1-(3-tert-butylphenyl)cyclohexylamino)-1-(3,5-difluorophenyl)-3-hydroxybutan-2-yl)-3-phenylpropanamide, N-(4-(1-(3-tert-butylphenyl)cyclohexylamino)-1-(3,5-difluorophenyl)-3-hydroxybutan-2-yl)-3-(furan-2-yl)propanamide, (E)-N-(4-(1-(3-tert-butylphenyl)cyclohexylamino)-1-(3,5-difluorophenyl)-3-hydroxybutan-2-yl)-3-(pyridin-3-yl)acrylamide, N-(4-(1-(3-tert-butylphenyl)cyclohexylamino)-1-(3,5-difluorophenyl)-3-hydroxybutan-2-yl)-4-oxo-4-(thiophen-2-yl)butanamide, N-(4-(1-(3-tert-butylphenyl)cyclohexylamino)-1-(3,5-difluorophenyl)-3-hydroxybutan-2-yl)-3-(pyridin-3-yl)propanamide, N-(4-(1-(3-tert-butylphenyl)cyclohexylamino)-1-(3,5-difluorophenyl)-3-hydroxybutan-2-yl)-2-hydroxybutanamide, N-(4-(1-(3-tert-butylphenyl)cyclohexylamino)-1-(3,5-difluorophenyl)-3-hydroxybutan-2-yl)-4-oxo-4-phenylbutanamide, N-(4-(1-(3-tert-butylphenyl)cyclohexylamino)-1-(3,5-difluorophenyl)-3-hydroxybutan-2-yl)-2-hydroxy-2-methylpropanamide, N-(4-(1-(3-tert-butylphenyl)cyclohexylamino)-1-(3,5-difluorophenyl)-3-hydroxybutan-2-yl)-4-oxopentanamide, N-(4-(1-(3-tert-butylphenyl)cyclohexylamino)-1-(3,5-difluorophenyl)-3-hydroxybutan-2-yl)-5-oxo-5-phenylpentanamide, N-(4-(1-(3-tert-butylphenyl)cyclohexylamino)-1-(3,5-difluorophenyl)-3-hydroxybutan-2-yl)-5-oxohexanamide, N-(4-(1-(3-tert-butylphenyl)cyclohexylamino)-1-(3,5-difluorophenyl)-3-hydroxybutan-2-yl)-6-oxoheptanamide, N-(4-(1-(3-tert-butylphenyl)cyclohexylamino)-1-(3,5-difluorophenyl)-3-hydroxybutan-2-yl)-1H-pyrazole-4-carboxamide, N-(4-(1-(3-tert-butylphenyl)cyclohexylamino)-1-(3,5-difluorophenyl)-3-hydroxybutan-2-yl)thiazole-5-carboxamide, N-(4-(1-(3-tert-butylphenyl)cyclohexylamino)-1-(3,5-difluorophenyl)-3-hydroxybutan-2-yl)-3,5-dimethylisoxazole-4-carboxamide, N-(4-(1-(3-tert-butylphenyl)cyclohexylamino)-1-(3,5-difluorophenyl)-3-hydroxybutan-2-yl)-2-oxothiazolidine-4-carboxamide, tert-butyl 3-(4-(1-(3-tert-butylphenyl)cyclohexylamino)-1-(3,5-difluorophenyl)-3-hydroxybutan-2-ylcarbamoyl)azetidine-1-carboxylate, 5-Oxo-tricyclo[2.2.1.02,6]heptane-3-carboxylic acid [3-[1-(3-tert-butyl-phenyl)-cyclohexylamino]-1-(3,5-difluoro-benzyl)-2-hydroxy-propyl]-amide, benzyl 4-(3-(tert-butoxycarbonyl)-4-(3,5-difluorophenyl)-2-hydroxybutylamino)-4-(3-tert-butylphenyl)piperidine-1-carboxylate, N-(4-(1-(3-tert-butylphenyl)cyclohexylamino)-1-(3,5-difluorophenyl)-3-hydroxybutan-2-yl)-5-oxopyrrolidine-2-carboxamide, N-(4-(1-(3-tert-butylphenyl)cyclohexylamino)-1-(3,5-difluorophenyl)-3-hydroxybutan-2-yl)-1-methylpyrrolidine-2-carboxamide, N-(4-(1-(3-tert-butylphenyl)cyclohexylamino)-1-(3,5-difluorophenyl)-3-hydroxybutan-2-yl)-3-(1H-imidazol-5-yl)propanamide, N-(4-(1-(3-tert-butylphenyl)cyclohexylamino)-1-(3,5-difluorophenyl)-3-hydroxybutan-2-yl)-2-acetamido-3-hydroxypropanamide, N-(4-(1-(3-tert-butylphenyl)cyclohexylamino)-1-(3,5-difluorophenyl)-3-hydroxybutan-2-yl)-1-acetylpyrrolidine-2-carboxamide, +N-(4-(1-(3-tert-butylphenyl)cyclohexylamino)-1-(3,5-difluorophenyl)-3-hydroxybutan-2-yl)-1H-tetrazole-1-carboxamide, 4-(4-(1-(3-tert-butylphenyl)cyclohexylamino)-1-(3,5-difluorophenyl)-3-hydroxybutan-2-ylcarbamoyl)-2,2-dimethylbutanoic acid, 4-[3-[1-(3-tert-Butyl-phenyl)-cyclohexylamino]-1-(3,5-difluoro-benzyl)-2-hydroxy-propylcarbamoyl]-adamantane-1-carboxylic acid methyl ester, N-(4-(1-(3-tert-butylphenyl)cyclohexylamino)-1-(3,5-difluorophenyl)-3-hydroxybutan-2-yl)-3-(pyridin-4-yl)propanamide, 2-((4-(1-(3-tert-butylphenyl)cyclohexylamino)-1-(3,5-difluorophenyl)-3-hydroxybutan-2-ylcarbamoyl)methoxy)acetic acid, 3-(4-(1-(3-tert-butylphenyl)cyclohexylamino)-1-(3,5-difluorophenyl)-3-hydroxybutan-2-ylcarbamoyl)cyclohexanecarboxylic acid, methyl 4-(4-(1-(3-tert-butylphenyl)cyclohexylamino)-1-(3,5-difluorophenyl)-3-hydroxybutan-2-ylcarbamoyl)-4-methylpentanoate, methyl 2-(2-((4-(1-(3-tert-butylphenyl)cyclohexylamino)-1-(3,5-difluorophenyl)-3-hydroxybutan-2-yl)carbamoyl)pyrrolidin-1-yl)acetate, 1-(2-amino-2-oxoethyl)-N-(4-(1-(3-tert-butylphenyl)cyclohexylamino)-1-(3,5-difluorophenyl)-3-hydroxybutan-2-yl)pyrrolidine-2-carboxamide, 2-(2-((4-(1-(3-tert-butylphenyl)cyclohexylamino)-1-(3,5-difluorophenyl)-3-hydroxybutan-2-yl)carbamoyl)pyrrolidin-1-yl)acetic acid, tert-butyl 4-(tert-butoxycarbonyl)-5-(4-(1-(3-tert-butylphenyl)cyclohexylamino)-1-(3,5-difluorophenyl)-3-hydroxybutan-2-ylamino)-5-oxopentanoate, tert-butyl 4-(1-(4-bromo-3-tert-butylphenyl)cyclohexylamino)-1-(3,5-difluorophenyl)-3-hydroxybutan-2-ylcarbamate, 1-(4-(1-(3-tert-butylphenyl)cyclohexylamino)-1-(3,5-difluorophenyl)-3-hydroxybutan-2-yl)urea, tert-butyl 4-(1-(3-tert-butylphenyl)-4-methoxycyclohexylamino)-1-(3,5-difluorophenyl)-3-hydroxybutan-2-ylcarbamate, tert-butyl 1-(3,5-difluorophenyl)-3-hydroxy-4-(1-(3-(methylthio)phenyl)cyclohexylamino)butan-2-ylcarbamate, N-(4-(1-(3-tert-butylphenyl)cyclohexylamino)-1-(3,5-difluorophenyl)-3-hydroxybutan-2-yl)-3-oxo-2-oxa-bicyclo[2.2.1]heptane-1-carboxamide, 5-Oxo-pyrrolidine-2-carboxylic acid [3-[1-(3-tert-butyl-phenyl)-cyclohexylamino]-1-(3,5-difluoro-benzyl)-2-hydroxy-propyl]-amide, 1-(4-(1-(3-tert-butylphenyl)cyclohexylamino)-1-(3,5-difluorophenyl)-3-hydroxybutan-2-yl)-3-hexylurea, 1-(4-(1-(3-tert-butylphenyl)cyclohexylamino)-1-(3,5-difluorophenyl)-3-hydroxybutan-2-yl)-3-cyclohexylurea, 1-(4-(1-(3-tert-butylphenyl)cyclohexylamino)-1-(3,5-difluorophenyl)-3-hydroxybutan-2-yl)-3-cyclopentylurea, ethyl 2-(3-(4-(1-(3-tert-butylphenyl)cyclohexylamino)-1-(3,5-difluorophenyl)-3-hydroxybutan-2-yl)ureido)-4-(methylthio)butanoate, N-(4-(1-(3-tert-butylphenyl)cyclohexylamino)-1-(3,5-difluorophenyl)-3-hydroxybutan-2-yl)-2,2,2-trifluoroacetamide, tert-butyl 4-(1-(3-tert-butyl-5-fluorophenyl)cyclohexylamino)-1-(3,5-difluorophenyl)-3-hydroxybutan-2-ylcarbamate, (4-(1-(3-tert-butylphenyl)cyclohexylamino)-1-(3,5-difluorophenyl)-3-hydroxybutan-2-yl)carbamic acid, 3-acetyl-1-butyl-N-(4-(1-(3-tert-butylphenyl)cyclohexylamino)-1-(3,5-difluorophenyl)-3-hydroxybutan-2-yl)-1H-indole-6-carboxamide, N-(4-(1-(3-tert-butylphenyl)cyclohexylamino)-1-(3,5-difluorophenyl)-3-hydroxybutan-2-yl)-3-(N-methylmethan-2-ylsulfonamido)benzamide, N-(4-(1-(3-tert-butylphenyl)cyclohexylamino)-1-(3,5-difluorophenyl)-3-hydroxybutan-2-yl)-5-(methylsulfonyl)thiophene-2-carboxamide, N-(4-(1-(3-tert-butylphenyl)cyclohexylamino)-1-(3,5-difluorophenyl)-3-hydroxybutan-2-yl)-2-(methylsulfonamido)thiazole-4-carboxamide, N-(4-(1-(3-tert-butylphenyl)cyclohexylamino)-1-(3,5-difluorophenyl)-3-hydroxybutan-2-yl)-3,5-dimethylisoxazole-4-sulfonamide, N-(4-(1-(3-tert-butylphenyl)cyclohexylamino)-1-(3,5-difluorophenyl)-3-hydroxybutan-2-yl)methanesulfonamide, and the like.

In another embodiment, the compound of formula (I) is selected from 2-((4-(1-(3-tert-butylphenyl)cyclohexylamino)-1-(3,5-difluorophenyl)-3-hydroxybutan-2-ylcarbamoyl)methoxy)acetic acid, 4-(4-(1-(3-tert-butylphenyl)cyclohexylamino)-1-(3,5-difluorophenyl)-3-hydroxybutan-2-ylcarbamoyl)-2,2-dimethylbutanoic acid, 4-[3-[1-(3-tert-Butyl-phenyl)-cyclohexylamino]-1-(3,5-difluoro-benzyl)-2-hydroxy-propylcarbamoyl]-butyric acid, N-(4-(1-(3-tert-butylphenyl)cyclohexylamino)-1-(3,5-difluorophenyl)-3-hydroxybutan-2-yl)-3-(N-methylmethan-2-ylsulfonamido)benzamide, Pentanedioic acid amide [3-[1-(3-tert-butyl-phenyl)-cyclohexylamino]-1-(3,5-difluoro-benzyl)-2-hydroxy-propyl]-amide, N-(4-(1-(3-tert-butylphenyl)cyclohexylamino)-1-(3,5-difluorophenyl)-3-hydroxybutan-2-yl)-2-(methylsulfonamido)thiazole-4-carboxamide, and the like.

An embodiment of the present invention is compounds of formula (I), 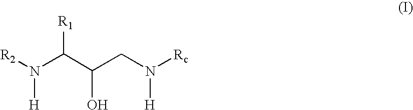
or pharmaceutically acceptable salts thereof, wherein R and R′ are independently selected from hydrogen and —C₁-C₁₀ alkyl (substituted with at least one group selected from OH).

In another embodiment, R_{B} is selected from —CF₃, —(CO)₀₋₁—(O)₀₋₁-alkyl, and —(CO)₀₋₁—OH.

In another embodiment, R_{N} is selected alkyl-R₁₀₀, —NH₂, —OH, —(CRR′)₁₋₆—P(O)(O-alkyl)₂, and alkyl-O-alkyl-C(O)OH.

In another embodiment, R_{N} is selected from alkyl-R₁₀₀, wherein the alkyl comprises at least one double or triple bond.

In another embodiment, R₄ and R_{4′} are independently selected from —OH.

In another embodiment, R₁₀₀ and R′₁₀₀ are independently selected from alkoxy.

In another embodiment, R₁₀₁ and R′₁₀₁ are independently selected from —(CO)₀₋₁—(O)₀₋₁-alkyl and —(CO)₀₋₁—OH.

In another embodiment, R₁₁₅ is —NH—C(O)-(alkyl).

In another embodiment, R₂₀₀ is —(CH₂)₀₋₄—C(O)—NH(R₂₁₅).

In another embodiment, R₂₀₅ is selected from —(CH₂)₀₋₆—C(O)—R₂₃₅, —(CH₂)₀₋₄—N(H or R₂₁₅)—SO₂—R₂₃₅, —CN, and —OCF₃.

In another embodiment, R₂₁₀ is selected from heterocycloalkyl, heteroaryl, —(CO)₀₋₁R₂₁₅, —(CO)₀₋₁R₂₂₀, —(CH₂)₀₋₄—NR₂₃₅R₂₄₀, —(CH₂)₀₋₄—NR₂₃₅(alkoxy), —(CH₂)₀₋₄—S—(R₂₁₅), —(CH₂)₀₋₆—OH, —(CH₂)₀₋₆—CN, —(CH₂)₀₋₄—NR₂₃₅—C(O)H, —(CH₂)₀₋₄—NR₂₃₅—C(O)-(alkoxy), —(CH₂)₀₋₄—NR₂₃₅—C(O)—R₂₄₀, and —C(O)—NHR₂₁₅.

In another embodiment, R₂₃₅ and R₂₄₀ are independently selected from —OH, —CF₃, —OCH₃, —NH—CH₃, —N(CH₃)₂, —(CH₂)₀₋₄—C(O)—(H or alkyl).

In another embodiment, at least one of A, B, and C is selected from —NH—and —N(R₂₀₀).

In another embodiment, D is cycloalkyl.

In another embodiment, E₁ is C₁-C₄ alkyl.

In another embodiment, V is cycloalkyl.

In another embodiment, at least one carbon of the aryl, heteroaryl, cycloalkyl, and heterocycloalkyl groups included within V and V′ are optionally replaced with a group selected from —C(O)—, —C(S)—, —C(═N—H)—, —C(═N—OH)—, —C(═N-alkyl)-, and —C(═N—O-alkyl)-, —(CO)₀₋₁—(O)₀₋₁-alkyl, and —(CO)₀₋₁—OH.

In another embodiment, the formula (I) compounds are selected from cyclopent-1-enecarboxylic acid [3-[1-(3-tert-butyl-phenyl)-cyclohexylamino]-1-(3,5-difluoro-benzyl)-2-hydroxy-propyl]-amide, cyclopropanecarbothioic acid [3-[1-(3-tert-butyl-phenyl)-cyclohexylamino]-1-(3,5-difluoro-benzyl)-2-hydroxy-propyl]-amide, 2-Oxo-imidazolidine-4-carboxylic acid [3-[1-(3-tert-butyl-phenyl)-cyclohexylamino]-1-(3,5-difluoro-benzyl)-2-hydroxy-propyl]-amide, 3-Acetylamino-N-[3-[1-(3-tert-butyl-phenyl)-cyclohexylamino]-1-(3,5-difluoro-benzyl)-2-hydroxy-propyl]-propionamide, 5-Acetylamino-pentanoic acid [3-[1-(3-tert-butyl-phenyl)-cyclohexylamino]-1-(3,5-difluoro-benzyl)-2-hydroxy-propyl]-amide, 1-[3-[1-(3-tert-Butyl-phenyl)-cyclohexylamino]-1-(3,5-difluoro-benzyl)-2-hydroxy-propyl]-3-(3,5-dimethyl-isoxazol-4-yl)-urea, 3-{3-[3-[l-(3-tert-Butyl-phenyl)-cyclohexylamino]-1-(3,5-difluoro-benzyl)-2-hydroxy-propyl]-ureido}-propionic acid ethyl ester, 2-{3-[3-[1-(3-tert-Butyl-phenyl)-cyclohexylamino]-1-(3,5-difluoro-benzyl)-2-hydroxy-propyl]-ureido}-3-methyl-butyric acid ethyl ester, 2-{3-[3-[1-(3-tert-Butyl-phenyl)-cyclohexylamino]-1-(3,5-difluoro-benzyl)-2-hydroxy-propyl]-ureido}-4-methyl-pentanoic acid ethyl ester, N-[3-[1-(3-tert-Butyl-phenyl)-cyclohexylamino]-1-(3,5-difluoro-benzyl)-2-hydroxy-propyl]-4-sulfamoyl-butyramide, 1-Methyl-cyclopropanecarboxylic acid [3-[1-(3-tert-butyl-phenyl)-cyclohexylamino]-1-(3,5-difluoro-benzyl)-2-hydroxy-propyl]-amide, tert-butyl (4-(1-(3-tert-butylphenyl)cyclohexylamino)-1-(3,5-difluorophenyl)-3-hydroxybutan-2-ylcarbamoyl)methylcarbamate, 4,7,7-Trimethyl-3-oxo-2-oxa-bicyclo[2.2.1 ]heptane-1-carboxylic acid [3-[1-(3-tert-butyl-phenyl)-cyclohexylamino]-1-(3,5-difluoro-benzyl)-2-hydroxy-propyl]-amide, {[3-[1-(3-tert-Butyl-phenyl)-cyclohexylamino]-1-(3,5-difluoro-benzyl)-2-hydroxy-propylcarbamoyl]-methyl}-phosphonic acid diethyl ester, N-[3-[l-(3-tert-Butyl-phenyl)-cyclohexylamino]-1-(3,5-difluoro-benzyl)-2-hydroxy-propyl]-2-(2,5-dioxo-imidazolidin-4-yl)-acetamide, (E)-N-(4-(1-(3-tert-butylphenyl)cyclohexylamino)-1-(3,5-difluorophenyl)-3-hydroxybutan-2-yl)-3-(pyridin-3-yl)acrylamide, N-(4-(1-(3-tert-butylphenyl)cyclohexylamino)-1-(3,5-difluorophenyl)-3-hydroxybutan-2-yl)-2-oxothiazolidine-4-carboxamide, tert-butyl 3-(4-(1-(3-tert-butylphenyl)cyclohexylamino)-1-(3,5-difluorophenyl)-3-hydroxybutan-2-ylcarbamoyl)azetidine-1-carboxylate, 5-Oxo-tricyclo[2.2.1.02,6]heptane-3-carboxylic acid [3-[1-(3-tert-butyl-phenyl)-cyclohexylamino]-1-(3,5-difluoro-benzyl)-2-hydroxy-propyl]-amide, N-(4-(1-(3-tert-butylphenyl)cyclohexylamino)-1-(3,5-difluorophenyl)-3-hydroxybutan-2-yl)-5-oxopyrrolidine-2-carboxamide, 2-((4-(1-(3-tert-butylphenyl)cyclohexylamino)-1-(3,5-difluorophenyl)-3-hydroxybutan-2-ylcarbamoyl)methoxy)acetic acid, 3-(4-(1-(3-tert-butylphenyl)cyclohexylamino)-1-(3,5-difluorophenyl)-3-hydroxybutan-2-ylcarbamoyl)cyclohexanecarboxylic acid, methyl 4-(4-(1-(3-tert-butylphenyl)cyclohexylamino)-1-(3,5-difluorophenyl)-3-hydroxybutan-2-ylcarbamoyl)-4-methylpentanoate, 1-(2-amino-2-oxoethyl)-N-(4-(1-(3-tert-butylphenyl)cyclohexylamino)-1-(3,5-difluorophenyl)-3-hydroxybutan-2-yl)pyrrolidine-2-carboxamide, tert-butyl 4-(tert-butoxycarbonyl)-5-(4-(1-(3-tert-butylphenyl)cyclohexylamino)-1-(3,5-difluorophenyl)-3-hydroxybutan-2-ylamino)-5-oxopentanoate, 1-(4-(1-(3-tert-butylphenyl)cyclohexylamino)-1-(3,5-difluorophenyl)-3-hydroxybutan-2-yl)urea, N-(4-(1-(3-tert-butylphenyl)cyclohexylamino)-1-(3,5-difluorophenyl)-3-hydroxybutan-2-yl)-3-oxo-2-oxa-bicyclo[2.2.1]heptane-1-carboxamide, 5-Oxo-pyrrolidine-2-carboxylic acid [3-[1-(3-tert-butyl-phenyl)-cyclohexylamino]-1-(3,5-difluoro-benzyl)-2-hydroxy-propyl]-amide, ethyl 2-(3-(4-(1-(3-tert-butylphenyl)cyclohexylamino)-1-(3,5-difluorophenyl)-3-hydroxybutan-2-yl)ureido)-4-(methylthio)butanoate, N-[3-[1-(3-tert-Butyl-phenyl)-cyclohexylamino]-1-(3,5-difluoro-benzyl)-2-hydroxy-propyl]-2-(2-imino-imidazolidin-1-yl)-acetamide and the like.

The present invention encompasses methods of treatment using compounds with structural characteristics designed for interacting with their target molecules. Such characteristics include at least one moiety capable of interacting with at least one subsite of beta-secretase. Such characteristics also include at least one moiety capable of enhancing the interaction between the target and at least one subsite of beta-secretase. It is preferred that the compounds of formula (I) are efficacious. For example, it is preferred that the compounds of formula (I) decrease the level of beta-secretase using low dosages of the compounds. Preferably, the compounds of formula (I) decrease the level of A-beta by at least 10% using dosages of about 100 mg/kg. It is more preferred that the compounds of formula (I) decrease the level of A-beta by at least 10% using dosages of less than 100 mg/kg. It is also more preferred that the compounds of formula (I) decrease the level of A-beta by greater than 10% using dosages of about 100 mg/kg. It is most preferred that the compounds of formula (I) decrease the level of A-beta by greater than 10% using dosages of less than 100 mg/kg.

In an embodiment, the host is a cell.

In another embodiment, the host is an animal.

In another embodiment, the host is human.

In another embodiment, at least one compound of formula (I), or a pharmaceutically acceptable salt thereof, is administered in combination with a pharmaceutically acceptable carrier or diluent.

In another embodiment, the pharmaceutical compositions comprising compounds of formula (I) can be used to treat a wide variety of disorders or conditions including Alzheimer's disease, Down's syndrome or Trisomy 21 (including mild cognitive impairment (MCI) Down's syndrome), hereditary cerebral hemorrhage with amyloidosis of the Dutch type, chronic inflammation due to amyloidosis, prion diseases (including Creutzfeldt-Jakob disease, Gerstmann-Straussler syndrome, kuru scrapie, and animal scrapie), Familial Amyloidotic Polyneuropathy, cerebral amyloid angiopathy, other degenerative dementias including dementias of mixed vascular and degenerative origin, dementia associated with Parkinson's disease, dementia associated with progressive supranuclear palsy and dementia associated with cortical basal degeneration, diffuse Lewy body type of Alzheimer's disease, and frontotemporal dementias with parkinsonism (FTDP).

In another embodiment, the condition is Alzheimer's disease.

In another embodiment, the condition is dementia.

When treating or preventing these diseases, the methods of the present invention can either employ the compounds of formula (I) individually or in combination, as is best for the patient.

In treating a patient displaying any of the conditions discussed above, a physician may employ a compound of formula (I) immediately and continue administration indefinitely, as needed. In treating patients who are not diagnosed as having Alzheimer's disease, but who are believed to be at substantial risk for it, the physician may start treatment when the patient first experiences early pre-Alzheimer's symptoms, such as memory or cognitive problems associated with aging. In addition, there are some patients who may be determined to be at risk for developing Alzheimer's disease through the detection of a genetic marker such as APOE4 or other biological indicators that are predictive for Alzheimer's disease and related conditions. In these situations, even though the patient does not have symptoms of the disease or condition, administration of the compounds of formula (I) may be started before symptoms appear, and treatment may be continued indefinitely to prevent or delay the onset of the disease. Similar protocols are provided for other diseases and conditions associated with amyloidosis, such as those characterized by dementia.

In an embodiment, a method of preventing or treating at least one condition associated with amyloidosis, comprises administering to a host a composition comprising a therapeutically effective amount of at least one compound of formula (I), which may include beta-secretase complexed with at least one compound of formula (I), or a pharmaceutically acceptable salt thereof.

One embodiment of the present invention provides a method of preventing or treating the onset of Alzheimer's disease comprising administering to a patient a therapeutically effective amount of at least one compound of formula (I), or a pharmaceutically acceptable salt thereof, wherein R₁, R₂, and R_{C} are as previously defined.

Another embodiment of the present invention provides a method of preventing or treating the onset of dementia comprising administering to a patient a therapeutically effective amount of at least one compound of formula (I), or a pharmaceutically acceptable salt thereof, wherein R₁, R₂, and R_{C} are as previously defined.

Another embodiment of the present invention provides a method of preventing or treating at least one condition associated with amyloidosis by administering to a host an effective amount of at least one compound of formula (I), or a pharmaceutically acceptable salt thereof, wherein R₁, R₂, and R_{C} are as previously defined.

Another embodiment of the present invention provides a method of preventing or treating Alzheimer's disease by administering to a host an effective amount of at least one compound of formula (I), or a pharmaceutically acceptable salt thereof, wherein R₁, R₂, and R_{C} are as previously defined.

Another embodiment of the present invention provides a method of preventing or treating dementia by administering to a host an effective amount of at least one compound of formula (I), or a pharmaceutically acceptable salt thereof, wherein R₁, R₂, and R_{C} are as previously defined.

Another embodiment of the present invention provides a method of inhibiting beta-secretase activity in a cell. This method comprises administering to the cell an effective amount of at least one compound of formula (I), or a pharmaceutically acceptable salt thereof, wherein R₁, R₂, and R_{C} are as previously defined.

Another embodiment of the present invention provides a method of inhibiting beta-secretase activity in a host. This method comprises administering to the host an effective amount of at least one compound of formula (I), or a pharmaceutically acceptable salt thereof, wherein R₁, R₂, and R_{C} are as previously defined.

Another embodiment of the present invention provides a method of inhibiting beta-secretase activity in a host. This method comprises administering to the host an effective amount of at least one compound of formula (I), or a pharmaceutically acceptable salt thereof, wherein R₁, R₂, and R_{C} are as previously defined, and wherein the host is a human.

Another embodiment of the present invention provides methods of affecting beta-secretase-mediated cleavage of amyloid precursor protein in a patient, comprising administering a therapeutically effective amount of at least one compound of formula (I), or a pharmaceutically acceptable salt thereof, wherein R₁, R₂, and R_{C} are as previously defined.

Another embodiment of the present invention provides a method of inhibiting cleavage of amyloid precursor protein at a site between Met596 and Asp597 (numbered for the APP-695 amino acid isotype), or at a corresponding site of an isotype or mutant thereof, comprising administering a therapeutically effective amount of at least one compound of formula (I), or a pharmaceutically acceptable salt thereof, wherein R₁, R₂, and R_{C} are as previously defined.

Another embodiment of the present invention provides a method of inhibiting cleavage of amyloid precursor protein or mutant thereof at a site between amino acids, comprising administering a therapeutically effective amount of at least one compound of formula (I), or a pharmaceutically acceptable salt thereof, wherein R₁, R₂, and R_{C} are as previously defined, and wherein the site between amino acids corresponds to between Met652 and Asp653 (numbered for the APP-751 isotype), between Met671 and Asp672 (numbered for the APP-770 isotype), between Leu596 and Asp597 of the APP-695 Swedish Mutation, between Leu652 and Asp653 of the APP-751 Swedish Mutation, or between Leu671 and Asp672 of the APP-770 Swedish Mutation.

Another embodiment of the present invention provides a method of inhibiting production of A-beta, comprising administering to a patient a therapeutically effective amount of at least one compound of formula (I), or a pharmaceutically acceptable salt thereof, wherein R₁, R₂, and R_{C} are as previously defined.

Another embodiment of the present invention provides a method of preventing or treating deposition of A-beta, comprising administering a therapeutically effective amount of at least one compound of formula (I), or a pharmaceutically acceptable salt thereof, wherein R₁, R₂, and R_{C} are as previously defined.

Another embodiment of the present invention provides a method of preventing, delaying, halting, or reversing a disease characterized by A-beta deposits or plaques, comprising administering a therapeutically effective amount of at least one compound of formula (I), or a pharmaceutically acceptable salt thereof, wherein R₁, R₂, and R_{C} are as previously defined.

In an embodiment, the A-beta deposits or plaques are in a human brain.

Another embodiment of the present invention provides a method of preventing, delaying, halting, or reversing a condition associated with a pathological form of A-beta in a host comprising administering to a patient in need thereof an effective amount of at least one compound of formula (I), or a pharmaceutically acceptable salt thereof, wherein R₁, R₂, and R_{C} are as previously defined.

Another embodiment of the present invention provides a method of inhibiting the activity of at least one aspartyl protease in a patient in need thereof, comprising administering a therapeutically effective amount of at least one compound of formula (I), or a pharmaceutically acceptable salt thereof to the patient, wherein R₁, R₂, and R_{C} are as previously defined.

In an embodiment, the at least one aspartyl protease is beta-secretase.

Another embodiment of the present invention provides a method of interacting an inhibitor with beta-secretase, comprising administering to a patient in need thereof a therapeutically effective amount of at least one compound of formula (I), or a pharmaceutically acceptable salt thereof, wherein R₁, R₂, and R_{C} are as previously defined, and wherein the at least one compound interacts with at least one beta-secretase subsite such as S1, S1′, or S2′.

Another embodiment of the present invention provides a method of selecting compounds of formula (I) wherein the pharmacokinetic parameters of are adjusted for an increase in desired effect (e.g., increased brain uptake).

Another embodiment of the present invention provides a method of selecting compounds of formula (I) wherein Cₘₐₓ, Tₘₐₓ, and/or half-life are adjusted to provide for maximum efficacy.

Another embodiment of the present invention provides a method of treating a condition in a patient, comprising administering a therapeutically effective amount of at least one compound of formula (I), or a pharmaceutically acceptable salt, derivative or biologically active metabolite thereof, to the patient, wherein R₁, R₂, and R_{C} are as previously defined.

In an embodiment, the condition is Alzheimer's disease.

In another embodiment, the condition is dementia.

In another embodiment of the present invention, the compounds of formula (I) are administered in oral dosage form. The oral dosage forms are generally administered to the patient 1, 2, 3, or 4 times daily. It is preferred that the compounds be administered either three or fewer times daily, more preferably once or twice daily. It is preferred that, whatever oral dosage form is used, it be designed so as to protect the compounds from the acidic environment of the stomach. Enteric coated tablets are well known to those skilled in the art. In addition, capsules filled with small spheres, each coated to be protected from the acidic stomach, are also well known to those skilled in the art.

Therapeutically effective amounts include, for example, oral administration from about 0.1 mg/day to about 1,000 mg/day, parenteral, sublingual, intranasal, intrathecal administration from about 0.2 mg/day to about 100 mg/day, depot administration and implants from about 0.5 mg/day to about 50 mg/day, topical administration from about 0.5 mg/day to about 200 mg/day, and rectal administration from about 0.5 mg/day to about 500 mg/day.

When administered orally, an administered amount therapeutically effective to inhibit beta-secretase activity, to inhibit A-beta production, to inhibit A-beta deposition, or to treat or prevent Alzheimer's disease is from about 0.1 mg/day to about 1,000 mg/day.

In various embodiments, the therapeutically effective amount may be administered in, for example, pill, tablet, capsule, powder, gel, or elixir form, and/or combinations thereof. It is understood that, while a patient may be started at one dose or method of administration, that dose or method of administration may vary over time as the patient's condition changes.

A further embodiment of the present invention provides a method of prescribing a medication for preventing, delaying, halting, or reversing at least one disorder, condition or disease associated with amyloidosis. The method includes identifying in a patient symptoms associated with at least one disorder, condition or disease associated with amyloidosis, and prescribing at least one dosage form of at least one compound of formula (I), or a pharmaceutically acceptable salt, to the patient, wherein R₁, R₂, and R_{C} are as previously defined.

A further embodiment of the present invention provides an article of manufacture, comprising (a) at least one dosage form of at least one compound of formula (I), or a pharmaceutically acceptable salt thereof, wherein R₁, R₂, and R_{C} are as previously defined, (b) a package insert providing that a dosage form comprising a compound of formula (I) should be administered to a patient in need of therapy for at least one disorder, condition or disease associated with amyloidosis, and (c) at least one container in which at least one dosage form of at least one compound of formula (I) is stored.

A further embodiment of the present invention provides a packaged pharmaceutical composition for treating at least one condition related to amyloidosis, comprising (a) a container which holds an effective amount of at least one compound of formula (I), or a pharmaceutically acceptable salt thereof, and (b) instructions for using the pharmaceutical composition.

A further embodiment of the present invention provides an article of manufacture, comprising (a) a therapeutically effective amount of at least one compound of formula (I), or a pharmaceutically acceptable salt thereof, wherein R₁, R₂, and R_{C} are as previously defined, (b) a package insert providing an oral dosage form should be administered to a patient in need of therapy for at least one disorder, condition or disease associated with amyloidosis, and (c) at least one container comprising at least one oral dosage form of at least one compound of formula (I).

A further embodiment of the present invention provides an article of manufacture, comprising (a) at least one oral dosage form of at least one compound of formula (I), or a pharmaceutically acceptable salt thereof, wherein R₁, R₂, and R_{C} are as previously defined, in a dosage amount ranging from about 2 mg to about 1000 mg, associated with (b) a package insert providing that an oral dosage form comprising a compound of formula (I) in a dosage amount ranging from about 2 mg to about 1000 mg should be administered to a patient in need of therapy for at least one disorder, condition or disease associated with amyloidosis, and (c) at least one container in which at least one oral dosage form of at least one compound of formula (I) in a dosage amount ranging from about 2 mg to about 1000 mg is stored.

A further embodiment of the present invention provides an article of manufacture, comprising (a) at least one oral dosage form of at least one compound of formula (I) in a dosage amount ranging from about 2 mg to about 1000 mg in combination with (b) at least one therapeutically active agent, associated with (c) a package insert providing that an oral dosage form comprising a compound of formula (I) in a dosage amount ranging from about 2 mg to about 1000 mg in combination with at least one therapeutically active agent should be administered to a patient in need of therapy for at least one disorder, condition or disease associated with amyloidosis, and (d) at least one container in which at least one dosage form of at least one compound of formula (I) in a dosage amount ranging from about 2 mg to about 1000 mg in combination with a therapeutically active agent is stored.

A further embodiment of the present invention provides an article of manufacture, comprising (a) at least one parenteral dosage form of at least one compound of formula (I), or a pharmaceutically acceptable salt thereof, in a dosage amount ranging from about 0.2 mg/mL to about 50 mg/mL, associated with (b) a package insert providing that a parenteral dosage form comprising a compound of formula (I) in a dosage amount ranging from about 0.2 mg/mL to about 50 mg/mL should be administered to a patient in need of therapy for at least one disorder, condition or disease associated with amyloidosis, and (c) at least one container in which at least one parenteral dosage form of at least one compound of formula (I), or a pharmaceutically acceptable salt thereof, in a dosage amount ranging from about 0.2 mg/mL to about 50 mg/mL is stored.

A further embodiment of the present invention provides an article of manufacture comprising (a) a medicament comprising an effective amount of at least one compound of formula (I) or a pharmaceutically acceptable salt thereof, in combination with active and/or inactive pharmaceutical agents, (b) a package insert providing that an effective amount of at least one compound of formula (I) should be administered to a patient in need of therapy for at least one disorder, condition or disease associated with amyloidosis, and (c) a container in which a medicament comprising an effective amount of at least one compound of formula (I) in combination with a therapeutically active and/or inactive agent is stored.

In an embodiment, the therapeutically active agent is selected from an antioxidant, an anti-inflammatory, a gamma-secretase inhibitor, a neurotrophic agent, an acetyl cholinesterase inhibitor, a statin, an A-beta, and/or an anti-A-beta antibody.

Another embodiment of the present invention provides an article of manufacture comprising: (a) a medicament comprising: an effective amount of at least one compound of formula (I), 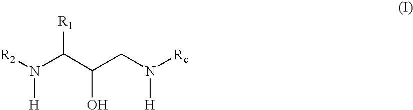
or a pharmaceutically acceptable salt thereof, wherein R₁, R₂, and R_{C} are defined bellow, in combination with active and/or inactive pharmaceutical agents; (b) a package insert providing that an effective amount of at least one compound of formula (I) should be administered to a patient in need of therapy for at least one disorder, condition or disease associated with amyloidosis; and (c) a container in which a medicament comprising: an effective amount of at least one compound of formula (I) in combination with active and/or inactive pharmaceutical agents is stored.

Another embodiment of the present invention provides A kit comprising: (a) at least one dosage form of at least one compound according to claim 1; and (b) at least one container in which at least one dosage form of at least one compound according to claim 1 is stored.

In an embodiment, the kit further comprises a package insert: a) containing information of the dosage amount and duration of exposure of a dosage form containing at least one compound of formula (I), or a pharmaceutically acceptable salt thereof, and b) providing that the dosage form should be administered to a patient in need of therapy for at least one disorder, condition or disease associated with amyloidosis.

In another embodiment, the kit further comprises at least one therapeutically active agent.

In another embodiment of a kit, the therapeutically active agent is selected from an antioxidant, an anti-inflammatory, a gamma-secretase inhibitor, a neurotrophic agent, an acetyl cholinesterase inhibitor, a statin, an A-beta, and an anti-A-beta antibody.

A further embodiment of the present invention provides method of preventing or treating at least one condition associated with amyloidosis, comprising: administering to a host a composition comprising a therapeutically effective amount of at least one selective beta-secretase inhibitor of formula (I), or a pharmaceutically acceptable salt thereof, further comprising a composition including beta-secretase complexed with at least one compound of formula (I), wherein R₁, R₂, and R_{C} are defined bellow, or pharmaceutically acceptable salt thereof.

A further embodiment of the present invention provides a method of producing a beta-secretase complex comprising exposing beta-secretase to a compound of formula (I), or a pharmaceutically acceptable salt thereof, in a reaction mixture under conditions suitable for the production of the complex.

A further embodiment of the present invention provides a manufacture of a medicament for preventing, delaying, halting, or reversing Alzheimer's disease, comprising adding an effective amount of at least one compound of formula (I), or a pharmaceutically acceptable salt thereof, wherein R₁, R₂, and R_{C} are defined bellow, to a pharmaceutically acceptable carrier.

A further embodiment of the present invention provides a method of selecting a beta-secretase inhibitor comprising targeting the moieties of at least one formula (I) compound, or a pharmaceutically acceptable salt thereof, to interact with at least one beta-secretase subsite such as, but not limited to, S1, S1′, or S2′.

The methods of treatment described herein include administering the compounds of formula (I) orally, parenterally (via intravenous injection (IV), intramuscular injection (IM), depo-IM, subcutaneous injection (SC or SQ), or depo-SQ), sublingually, intranasally (inhalation), intrathecally, topically, or rectally. Dosage forms known to those skilled in the art are suitable for delivery of the compounds of formula (I).

In treating or preventing the above diseases, the compounds of formula (I) are administered using a therapeutically effective amount. The therapeutically effective amount will vary depending on the particular compound used and the route of administration, as is known to those skilled in the art.

The compositions are preferably formulated as suitable pharmaceutical preparations, such as for example, pill, tablet, capsule, powder, gel, or elixir form, and/or combinations thereof, for oral administration or in sterile solutions or suspensions for parenteral administration. Typically the compounds described above are formulated into pharmaceutical compositions using techniques and/or procedures well known in the art.

For example, a therapeutically effective amount of a compound or mixture of compounds of formula (I), or a physiologically acceptable salt is combined with a physiologically acceptable vehicle, carrier, binder, preservative, stabilizer, flavor, and the like, in a unit dosage form as called for by accepted pharmaceutical practice and is defined herein. The amount of active substance in those compositions or preparations is such that a suitable dosage in the range indicated is obtained. The compound concentration is effective for delivery of an amount upon administration that lessens or ameliorates at least one symptom of the disorder for which the compound is administered. For example, the compositions can be formulated in a unit dosage form, each dosage containing from about 2 mg to about 1000 mg.

The active ingredient may be administered in a single dose, or may be divided into a number of smaller doses to be administered at intervals of time. It is understood that the precise dosage and duration of treatment is a function of the disease or condition being treated and may be determined empirically using known testing protocols or by extrapolation from in vivo or in vitro test data. It is to be noted that concentrations and dosage values may also vary with the severity of the condition to be alleviated. It is also to be understood that the precise dosage and treatment regimens may be adjusted over time according to the individual need and the professional judgment of the person administering or supervising the administration of the compositions, and that the concentration ranges set forth herein are exemplary only and are not intended to limit the scope or practice of the claimed compositions. A dosage and/or treatment method for any particular patient also may depend on, for example, the age, weight, sex, diet, and/or health of the patient, the time of administration, and/or any relevant drug combinations or interactions.

To prepare compositions to be employed in the methods of treatment, at least one compound of formula (I), or a pharmaceutically acceptable salt thereof, wherein R₁, R₂, and R_{C} are defined bellow, is mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. An effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. Additionally, the active materials can also be mixed with other active materials that do not impair the desired action, or with materials that supplement the desired action, or have another action. For example, the compounds of formula (I) may be formulated as the sole pharmaceutically active ingredient in the composition or may be combined with other active ingredients.

Where the compounds exhibit insufficient solubility, methods for solubilizing may be used. Such methods are known and include, for example, using co-solvents (such as dimethylsulfoxide (DMSO)), using surfactants (such as Tween®), and/or dissolution in aqueous sodium bicarbonate. Derivatives of the compounds, such as salts, metabolites, and/or pro-drugs, may also be used in formulating effective pharmaceutical compositions. Such derivatives may improve the pharmacokinetic properties of treatment administered.

A kit may include a plurality of containers, each container holding at least one unit dose of the compound of the present invention. The containers are preferably adapted for the desired mode of administration, including, for example, pill, tablet, capsule, powder, gel or gel capsule, sustained-release capsule, or elixir form, and/or combinations thereof and the like for oral administration, depot products, pre-filled syringes, ampoules, vials, and the like for parenteral administration, and patches, medipads, creams, and the like for topical administration.

The tablets, pills, capsules, troches, and the like may contain a binder (e.g., gum tragacanth, acacia, corn starch, gelatin, and the like); a vehicle (e.g., microcrystalline cellulose, starch, lactose, and the like); a disintegrating agent (e.g., alginic acid, corn starch, and the like); a lubricant (e.g., magnesium stearate, and the like); a gildant (e.g., colloidal silicon dioxide, and the like); a sweetening agent (e.g., sucrose, saccharin, and the like); a flavoring agent (e.g., peppermint, methyl salicylate, and the like); or fruit flavoring; compounds of a similar nature, and/or mixtures thereof.

When the dosage unit form is a capsule, it can contain, in addition to material described above, a liquid carrier such as a fatty oil. Additionally, dosage unit forms can contain various other materials, which modify the physical form of the dosage unit, for example, coatings of sugar or other enteric agents. A method of treatment can also administer the compound as a component of an elixir, suspension, syrup, wafer, chewing gum, or the like. A syrup may contain, in addition to the active compounds, sucrose as a sweetening agent, flavors, preservatives, dyes and/or colorings.

The methods of treatment may employ at least one carrier that protects the compound against rapid elimination from the body, such as time-release formulations or coatings. Such carriers include controlled release formulations, such as, for example, implants or microencapsulated delivery systems, and the like or biodegradable, biocompatible polymers such as collagen, ethylene vinyl acetate, polyanhydrides, polyglycolic acid, polyorthoesters, polylactic acid, and the like. Methods for preparation of such formulations are known to those in the art.

When orally administered, the compounds of the present invention can be administered in usual dosage forms for oral administration as is well known to those skilled in the art. These dosage forms include the usual solid unit dosage forms of tablets and capsules as well as liquid dosage forms such as solutions, suspensions, and elixirs. When solid dosage forms are used, it is preferred that they be of the sustained release type so that the compounds of the present invention need to be administered only once or twice daily. When liquid oral dosage forms are used, it is preferred that they be of about 10 mL to about 30 mL each. Multiple doses may be administered daily.

The methods of treatment may also employ a mixture of the active materials and other active or inactive materials that do not impair the desired action, or with materials that supplement the desired action.

Solutions or suspensions used for parenteral, intradermal, subcutaneous, or topical application can include a sterile diluent (e.g., water for injection, saline solution, fixed oil, and the like); a naturally occurring vegetable oil (e.g., sesame oil, coconut oil, peanut oil, cottonseed oil, and the like); a synthetic fatty vehicle (e.g., ethyl oleate, polyethylene glycol, glycerine, propylene glycol, and the like, including other synthetic solvents); antimicrobial agents (e.g., benzyl alcohol, methyl parabens, and the like); antioxidants (e.g., ascorbic acid, sodium bisulfite, and the like); chelating agents (e.g., ethylenediaminetetraacetic acid (EDTA), and the like); buffers (e.g., acetates, citrates, phosphates, and the like); and/or agents for the adjustment of tonicity (e.g., sodium chloride, dextrose, and the like); or mixtures thereof.

Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and the like, and mixtures thereof. Liposomal suspensions including tissue-targeted liposomes may also be suitable as pharmaceutically acceptable carriers. These may be prepared according to methods known, for example, as described in U.S. Pat. No. 4,522,811.

The methods of treatment include delivery of the compounds of the present invention in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The methods of treatment include administration of the compounds parenterally, for example, by IV, IM, SC, or depo-SC. When administered parenterally, a therapeutically effective amount of about 0.2 mg/mL to about 50 mg/mL is preferred. When a depot or IM formulation is used for injection once a month or once every two weeks, the preferred dose should be about 0.2 mg/mL to about 50 mg/mL.

The methods of treatment include administration of the compounds sublingually. When given sublingually, the compounds of the present invention should be given one to four times daily in the amounts described above for IM administration.

The methods of treatment include administration of the compounds intranasally. When given by this route, the appropriate dosage forms are a nasal spray or dry powder, as is known to those skilled in the art. The dosage of the compounds of the present invention for intranasal administration is the amount described above for IM administration.

The methods of treatment include administration of the compounds intrathecally. When given by this route the appropriate dosage form can be a parenteral dosage form as is known to those skilled in the art. The dosage of the compounds of the present invention for intrathecal administration is the amount described above for IM administration.

The methods of treatment include administration of the compounds topically. When given by this route, the appropriate dosage form is a cream, ointment, or patch. When topically administered, the dosage is from about 0.2 mg/day to about 200 mg/day. Because the amount that can be delivered by a patch is limited, two or more patches may be used. The number and size of the patch is not important. What is important is that a therapeutically effective amount of a compound of the present invention be delivered as is known to those skilled in the art. The compound can be administered rectally by suppository as is known to those skilled in the art. When administered by suppository, the therapeutically effective amount is from about 0.2 mg to about 500 mg.

The methods of treatment include administration of the compounds by implants as is known to those skilled in the art. When administering a compound of the present invention by implant, the therapeutically effective amount is the amount described above for depot administration.

Given a particular compound of the present invention and/or a desired dosage form and medium, one skilled in the art would know how to prepare and administer the appropriate dosage form and/or amount.

The methods of treatment include use of the compounds of the present invention, or acceptable pharmaceutical salts thereof, in combination, with each other or with other therapeutic agents, to treat or prevent the conditions listed above. Such agents or approaches include acetylcholine esterase inhibitors such as tacrine (tetrahydroaminoacridine, marketed as COGNEX®), donepezil hydrochloride, (marketed as Aricept®)) and rivastigmine (marketed as Exelon®), gamma-secretase inhibitors, anti-inflammatory agents such as cyclooxygenase 11 inhibitors, anti-oxidants such as Vitamin E or ginkolides, immunological approaches, such as, for example, immunization with A-beta peptide or administration of anti-A-beta peptide antibodies, statins, and direct or indirect neurotropic agents such as Cerebrolysin®, AIT-082 (Emilien, 2000, Arch. Neurol. 57:454), and other neurotropic agents, and complexes with beta-secretase or fragments thereof.

Additionally, some methods of treatment also employ the compounds of the present invention with inhibitors of P-glycoprotein (P-gp). P-gp inhibitors and the use of such compounds are known to those skilled in the art. See, for example, Cancer Research, 53, 4595-4602 (1993), Clin. Cancer Res., 2, 7-12 (1996), Cancer Research, 56, 4171-4179 (1996), International Publications WO 99/64001 and WO 01/10387. The blood level of the P-gp inhibitor should be such that it exerts its effect in inhibiting P-gp from decreasing brain blood levels of the compounds of formula (I). To that end the P-gp inhibitor and the compounds of formula (I) can be administered at the same time, by the same or different route of administration, or at different times. Given a particular compound of formula (I), one skilled in the art would know whether a P-gp inhibitor is desirable for use in the method of treatment, which P-gp inhibitor should be used, and how to prepare and administer the appropriate dosage form and/or amount.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 quinoline-3-carboxylic acid (2-{4-[2-(6,7-dimethyl-3,4-dihydro-1H-isoquinoline-2-yl)-ethyl]phenylcarbamoyl}-4,5-dimethylphenyl)-amide (Xenova), or other compounds. Compounds that have the same function and therefore achieve the same outcome are also considered to be useful.

The P-gp inhibitors can be administered orally, parenterally, (via IV, IM, depo-IM, SQ, depo-SQ), topically, sublingually, rectally, intranasally, intrathecally, or by implant.

The therapeutically effective amount of the P-gp inhibitors is from about 0.1 mg/kg to about 300 mg/kg daily, preferably about 0.1 mg/kg to about 150 mg/kg daily. It is understood that while a patient may be started on one dose, that dose may vary over time as the patient's condition changes.

When administered orally, the P-gp inhibitors can be administered in usual dosage forms for oral administration as is known to those skilled in the art. These dosage forms include the usual solid unit dosage forms of tablets or capsules as well as liquid dosage forms such as solutions, suspensions or elixirs. When the solid dosage forms are used, it is preferred that they be of the sustained release type so that the P-gp inhibitors need to be administered only once or twice daily. The oral dosage forms are administered to the patient one through four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that the dosage form used is designed to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered via IV, IM, depo-IM, SQ or depo-SQ.

The P-gp inhibitors can be given sublingually. When given sublingually, the P-gp inhibitors should be given one through four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the patch is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art.

The P-gp inhibitors can be administered rectally by suppository or by implants, both of which are known to those skilled in the art.

It should be apparent to one skilled in the art that the exact dosage and frequency of administration will depend on the particular compounds of the present invention administered, the particular condition being treated, the severity of the condition being treated, the age, weight, or general physical condition of the particular patient, or any other medication the individual may be taking as is well known to administering physicians who are skilled in this art.

Another embodiment of the present invention is to provide methods of preventing or treating at least one condition associated with amyloidosis using compounds with increased oral bioavailability (increased F values).

Accordingly, an embodiment of the present invention is also directed to methods for preventing or treating at least one condition associated with amyloidosis, comprising administering to a host, a therapeutically effective amount of at least one compound of formula (I), or a pharmaceutically acceptable salt thereof, wherein R₁, R₂, and R_{C} are as previously defined, and wherein the compound has an F value of at least 10%.

In another embodiment, the host is an animal. In another embodiment, the host is human.

In another embodiment, the F value is greater than about 20%. In yet a further embodiment, the F value is greater than about 30%.

A further embodiment of the present invention is to provide methods of preventing or treating at least one condition associated with amyloidosis using compounds with a high degree of selectivity.

Investigation of potential beta-secretase inhibitors produced compounds with increased selectivity for beta-secretase over other aspartyl proteases such as cathepsin D (catD), cathepsin E (catE), Human Immunodeficiency Viral (HIV) protease, and renin. Selectivity was calculated as a ratio of inhibition (IC₅₀) values in which the inhibition of beta-secretase was compared to the inhibition of other aspartyl proteases. A compound is selective when the IC₅₀ value (i.e., concentration required for 50% inhibition) of a desired target (e.g., beta-secretase) is less than the IC₅₀ value of a secondary target (e.g., catD).

Alternatively, a compound is selective when its binding affinity is greater for its desired target (e.g., beta-secretase) versus a secondary target (e.g., catD).

Accordingly, methods of treatment include administering selective compounds of formula (I) having a lower IC₅₀ value for inhibiting beta-secretase, or greater binding affinity for beta-secretase, than for other aspartyl proteases such as catD, catE, HIV protease, or renin. A selective compound is also capable of producing a higher ratio of desired effects to adverse effects, resulting in a safer method of treatment.

### EXAMPLE 1

### EXEMPLARY FORMULA (I) COMPOUNDS

| Example | |
|---|---|
| No. | Compound |
| 1-1. | 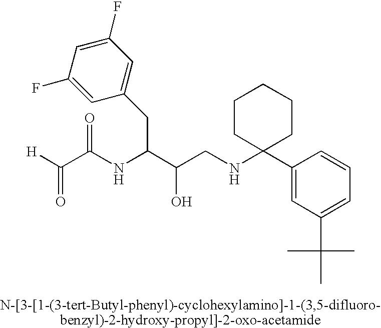 |
| 1-2. | 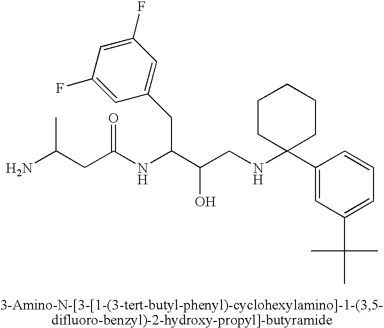 |
| 1-3. | 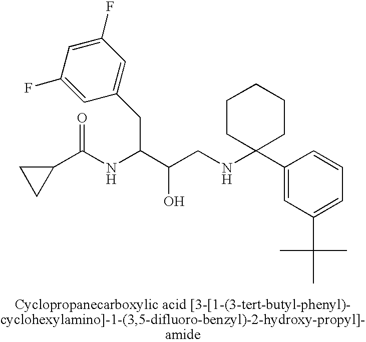 |
| 1-4. | 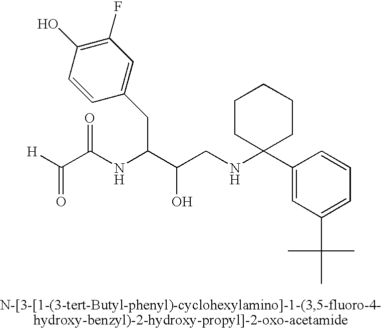 |
| 1-5. | 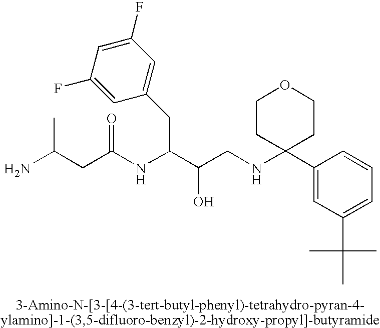 |
| 1-6. | 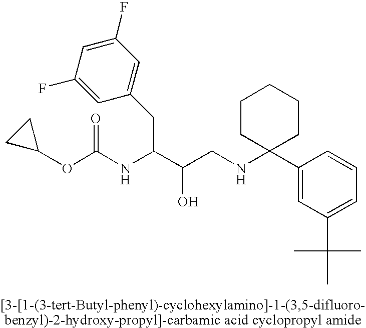 |
| 1-7. | 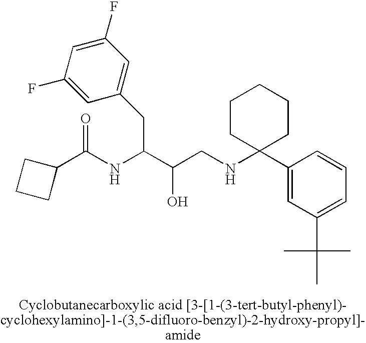 |
| 1-8. | 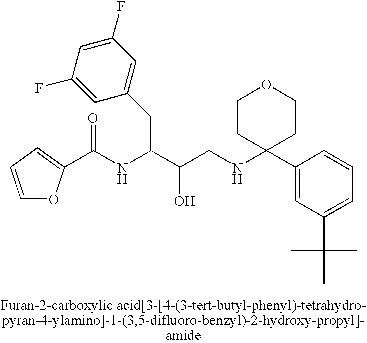 |
| 1-9. | 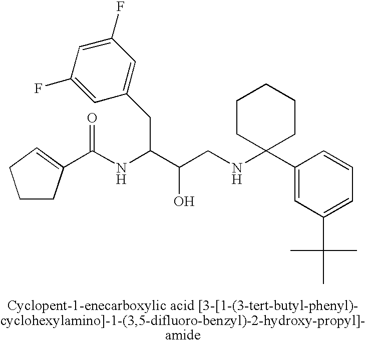 |
| 1-10. | 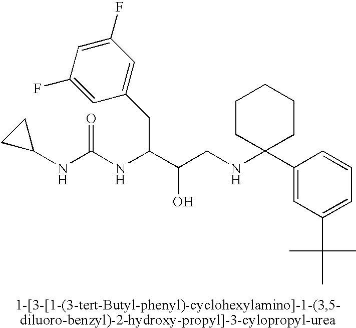 |
| 1-11. | 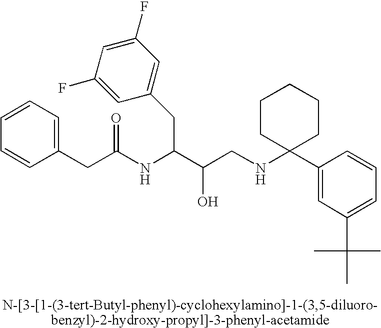 |
| 1-12. | 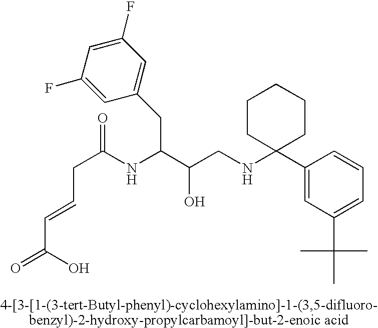 |
| 1-13. | 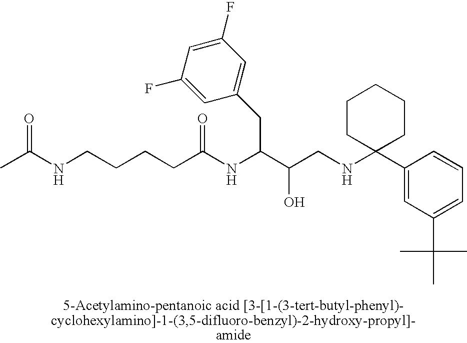 |
| 1-14. | 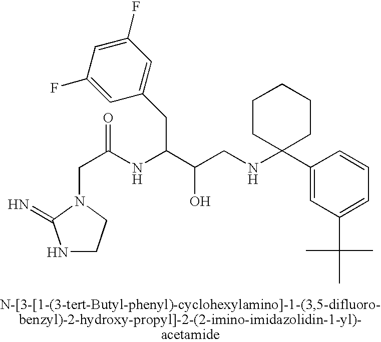 |
| 1-15. | 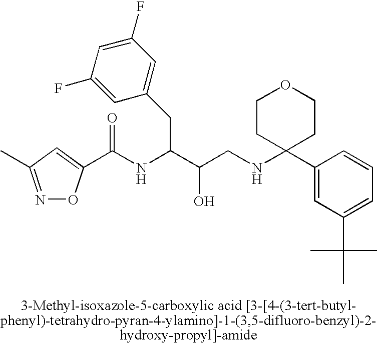 |
| 1-16. | 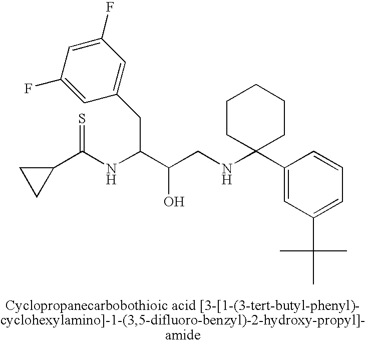 |
| 1-17. | 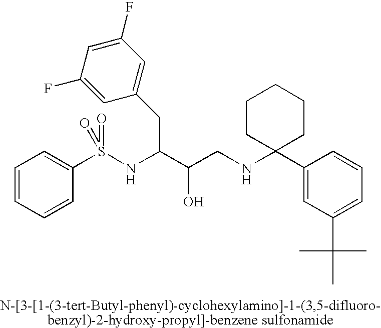 |
| 1-18. | 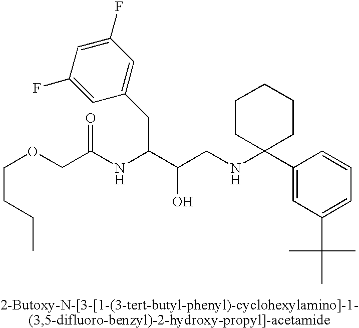 |
| 1-19. | 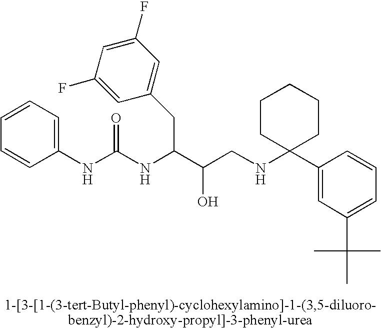 |
| 1-20. | 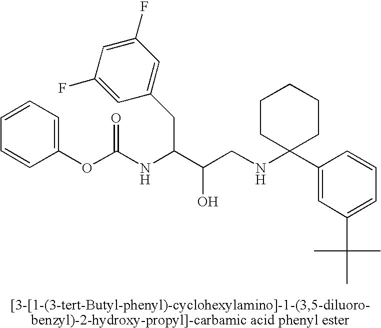 |
| 1-21. | 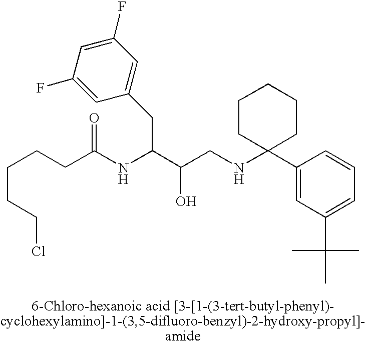 |
| 1-22. | 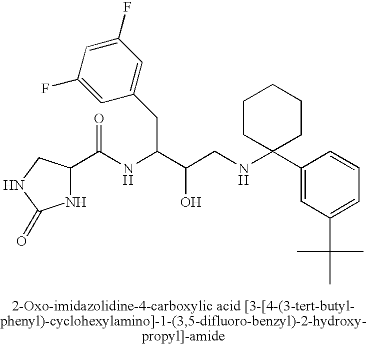 |
| 1-23. | 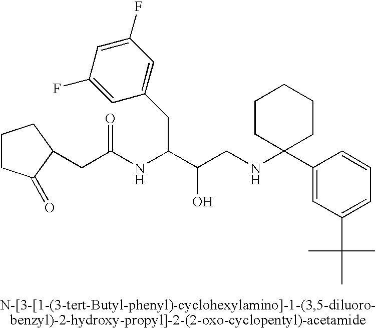 |
| 1-24. | 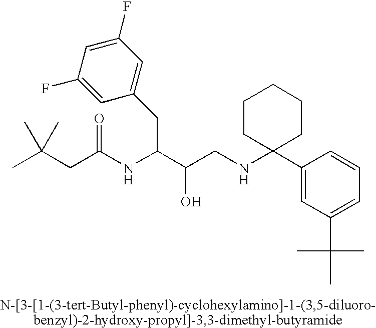 |
| 1-25. | 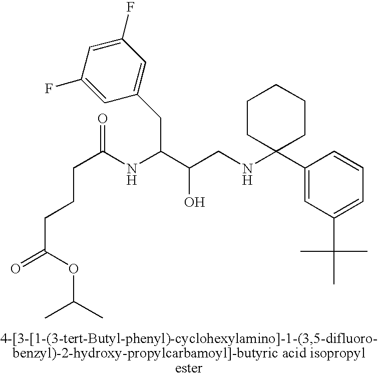 |
| 1-26. | 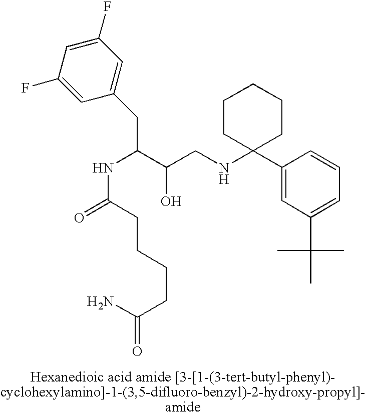 |
| 1-27. | 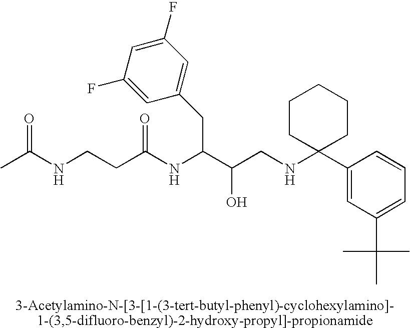 |
| 1-28. | 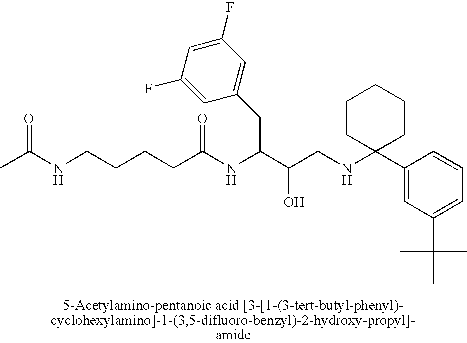 |
| 1-29. | 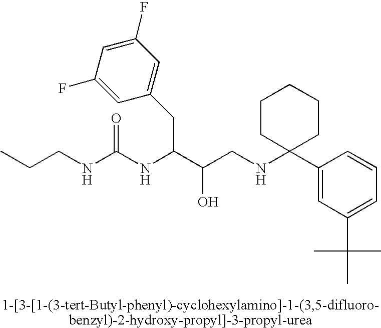 |
| 1-30. | 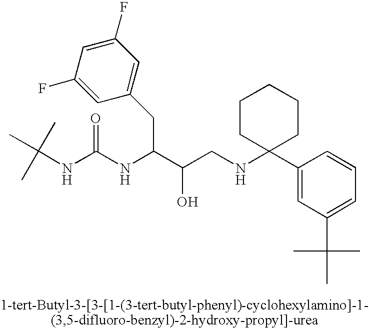 |
| 1-31. | 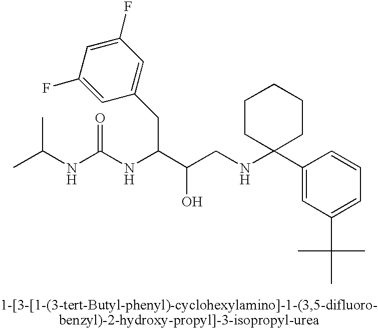 |
| 1-32. | 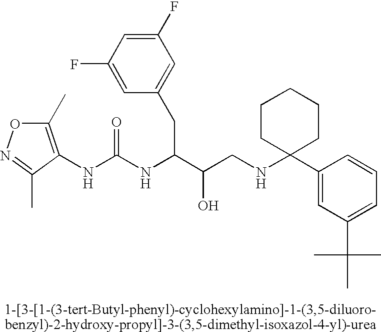 |
| 1-33. | 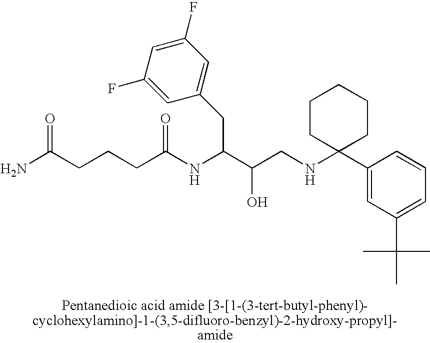 |
| 1-34. | 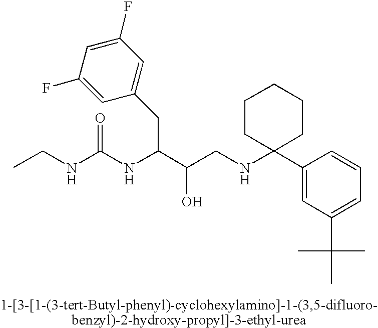 |
| 1-35. | 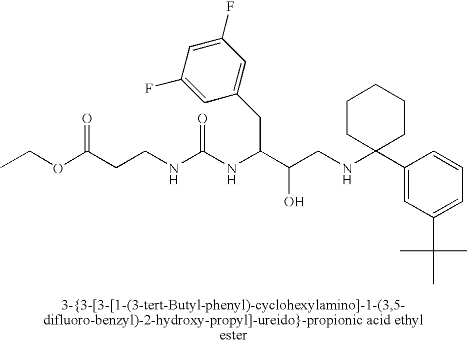 |
| 1-36. | 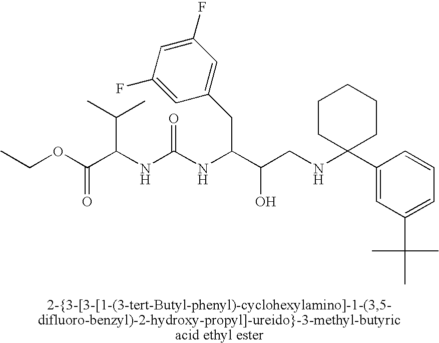 |
| 1-37. | 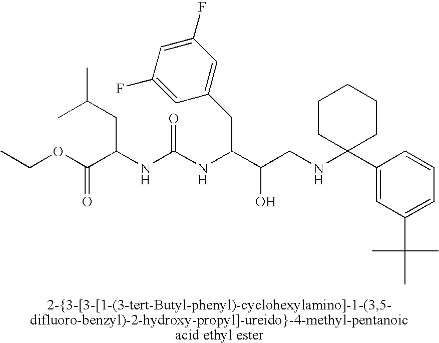 |
| 1-38. | 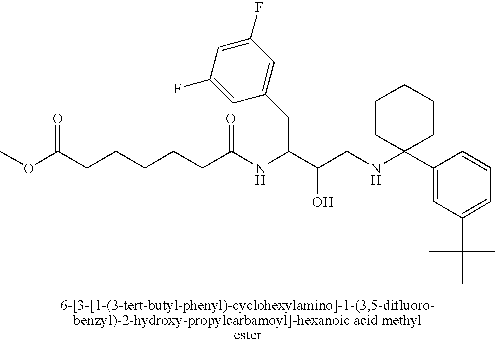 |
| 1-39. | 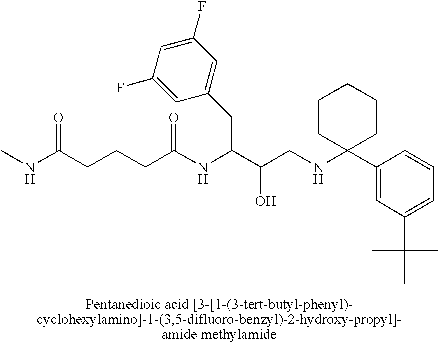 |
| 1-40. | 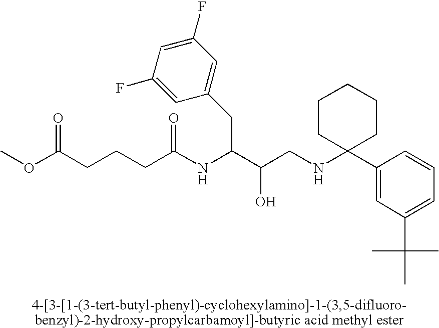 |
| 1-41. | 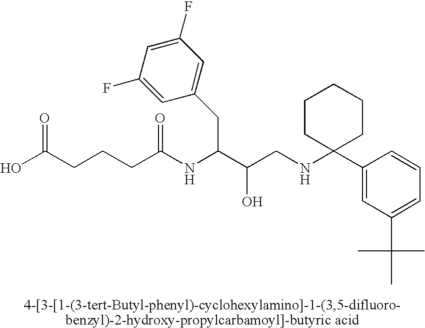 |
| 1-42. | 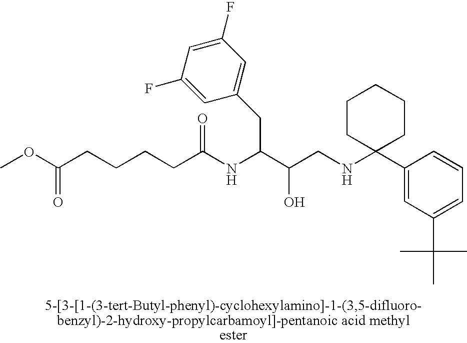 |
| 1-43. | 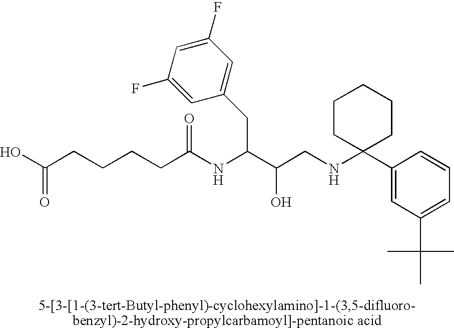 |
| 1-44. | 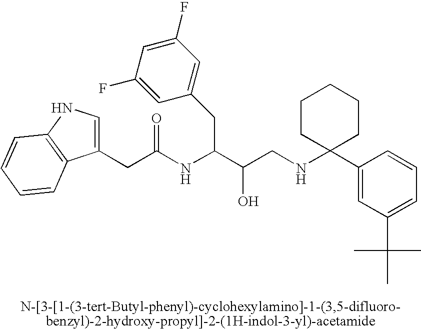 |
| 1-45. | 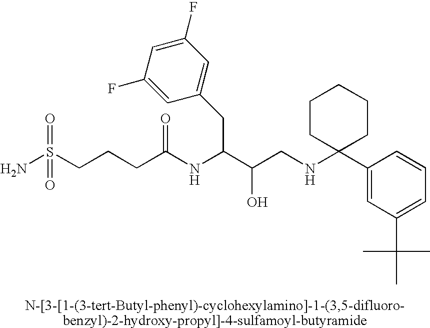 |
| 1-46. | 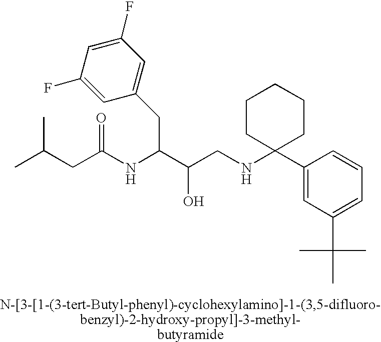 |
| 1-47. | 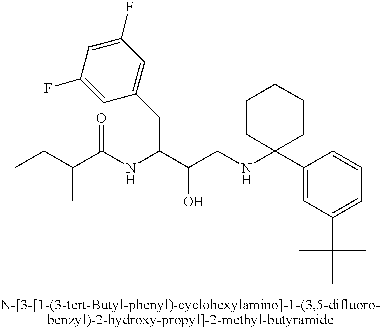 |
| 1-48. | 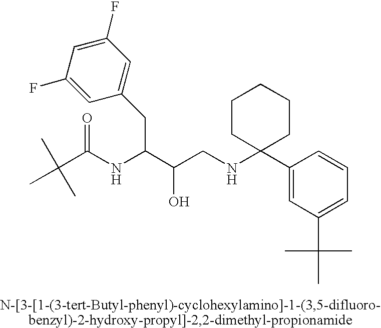 |
| 1-49. | 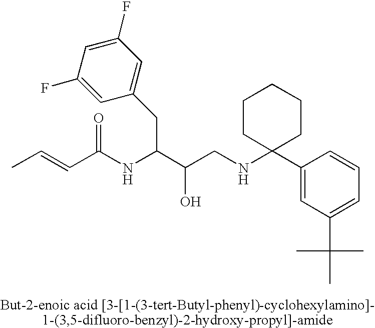 |
| 1-50. | 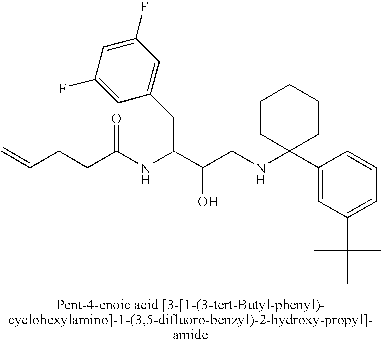 |
| 1-51. | 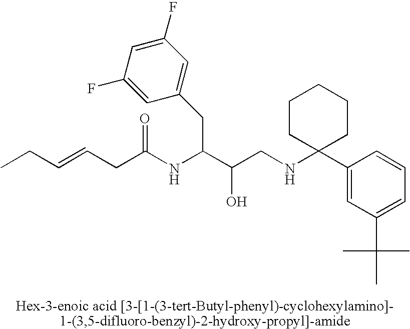 |
| 1-52. | 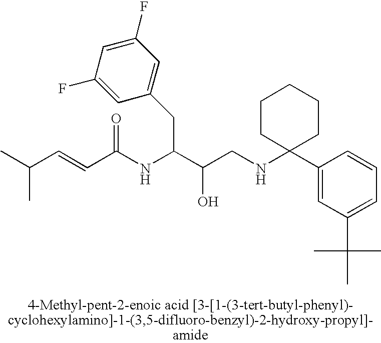 |
| 1-53. | 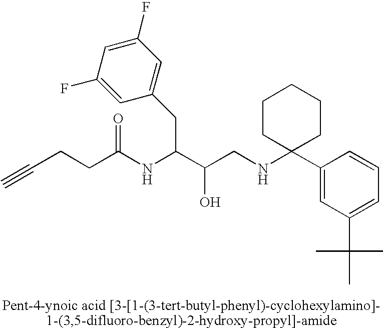 |
| 1-54. | 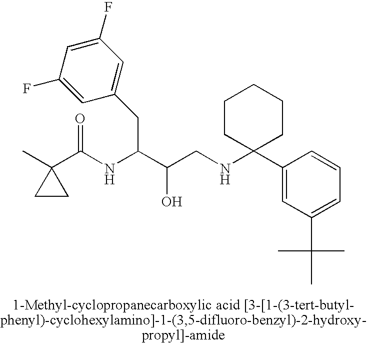 |
| 1-55. | 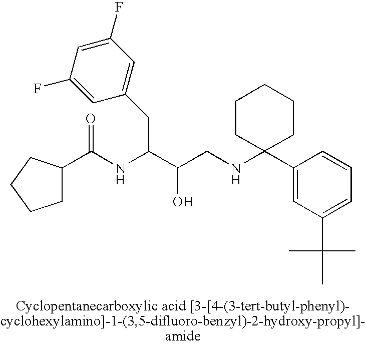 |
| 1-56. | 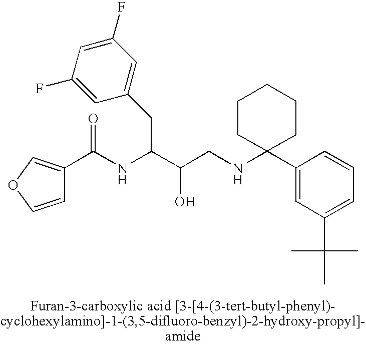 |
| 1-57. | 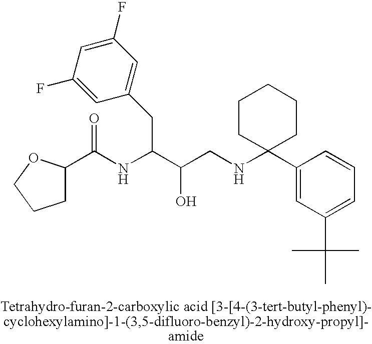 |
| 1-58. | 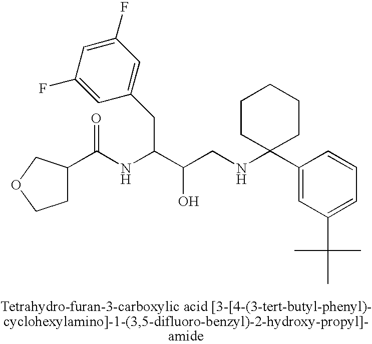 |
| 1-59. | 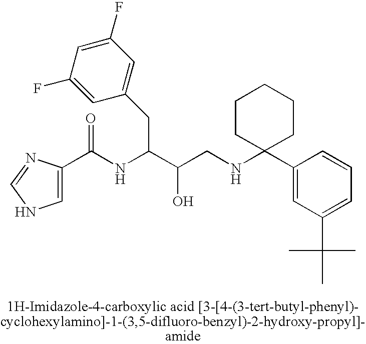 |
| 1-60. | 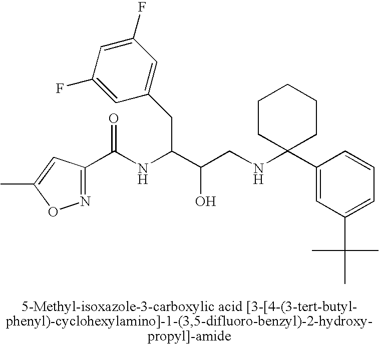 |
| 1-61. | 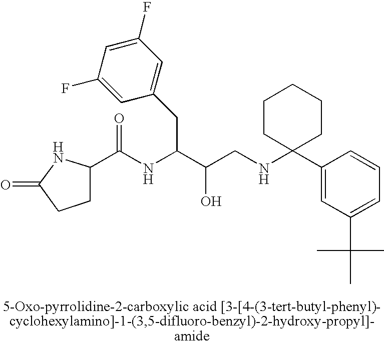 |
| 1-62. | 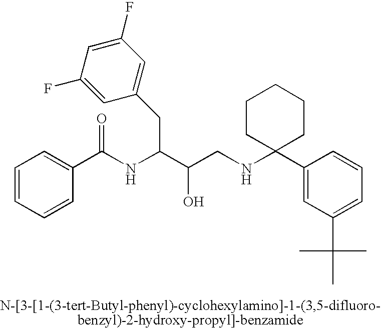 |
| 1-63. | 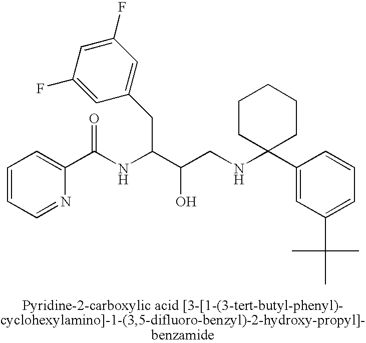 |
| 1-64. | 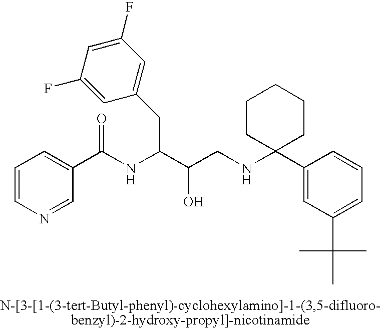 |
| 1-65. | 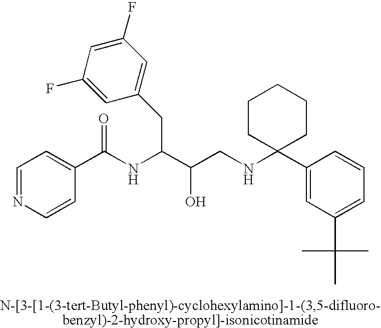 |
| 1-66. | 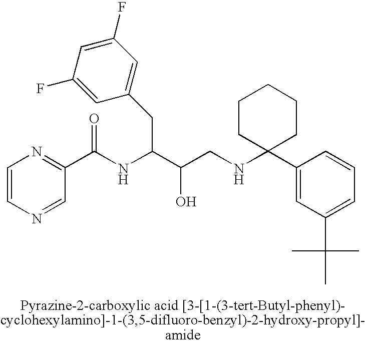 |
| 1-67. | 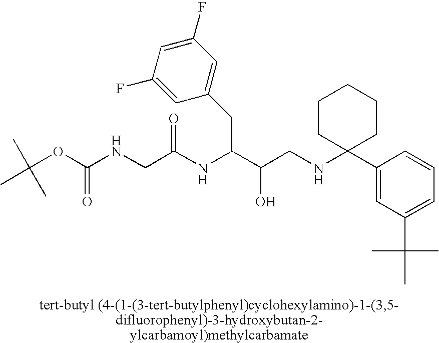 |
| 1-68. | 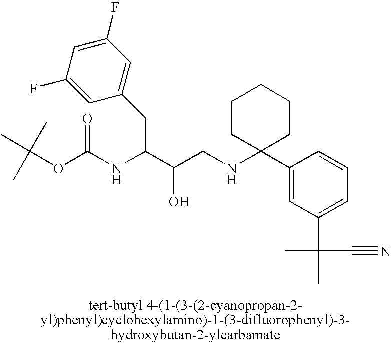 |
| 1-69. | 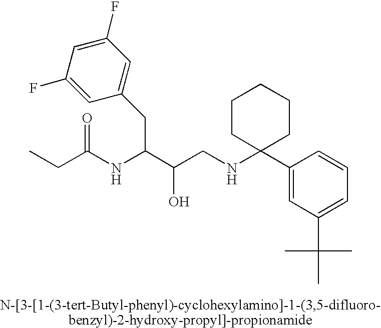 |
| 1-70. | 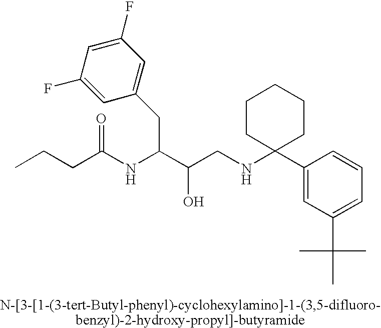 |
| 1-71. | 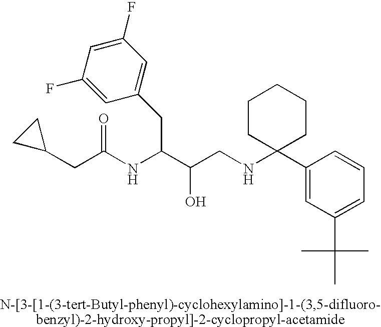 |
| 1-72. | 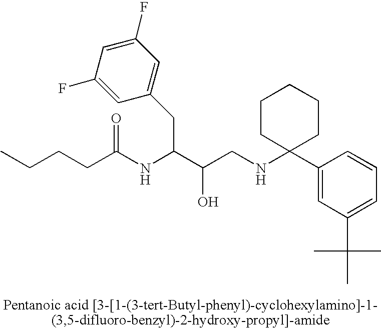 |
| 1-73. | 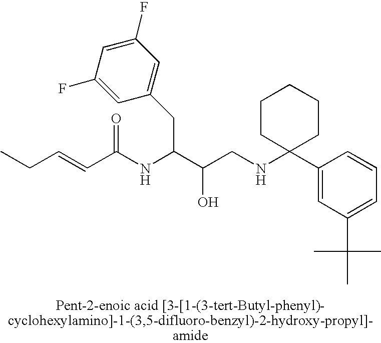 |
| 1-74. | 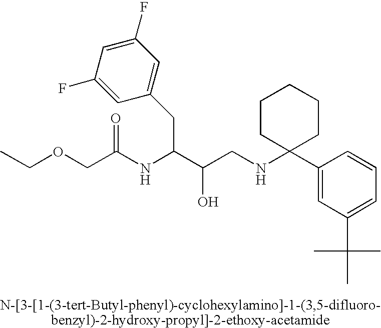 |
| 1-75. | 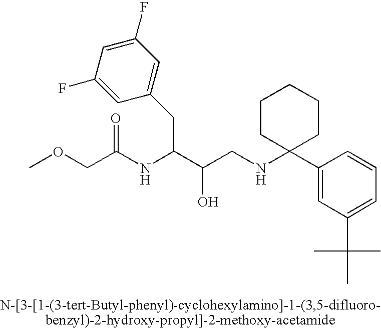 |
| 1-76. | 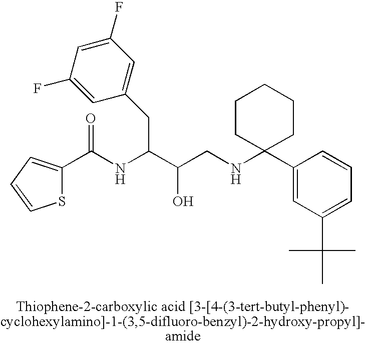 |
| 1-77. | 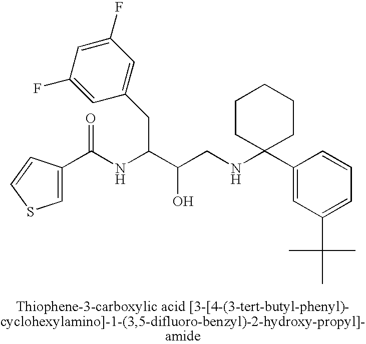 |
| 1-78. | 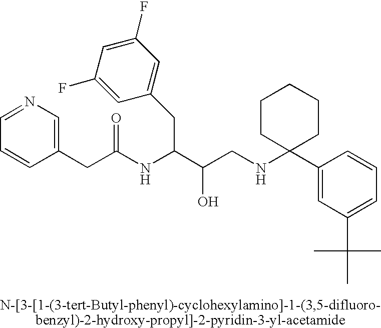 |
| 1-79. | 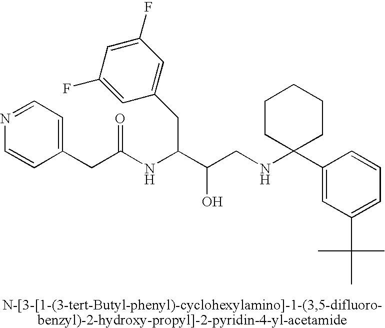 |
| 1-80. | 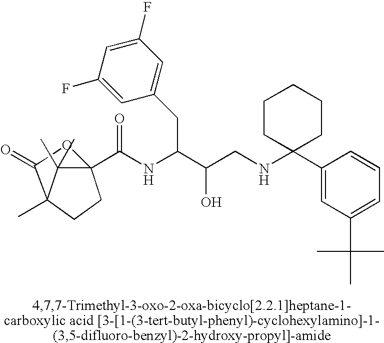 |
| 1-81. | 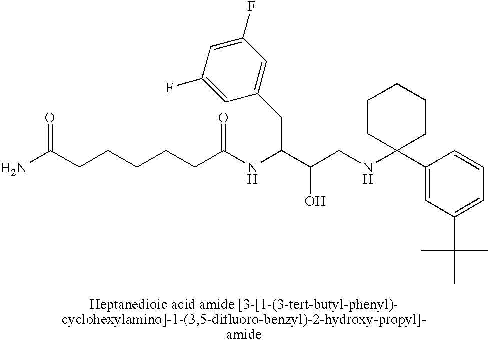 |
| 1-82. | 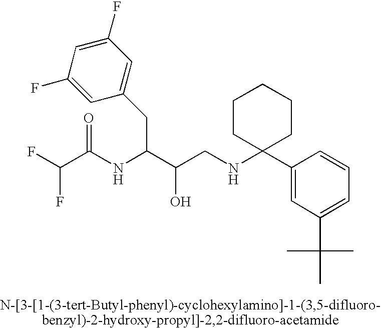 |
| 1-83. | 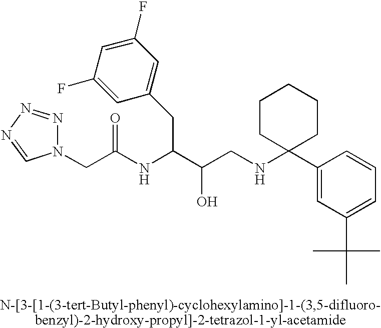 |
| 1-84. | 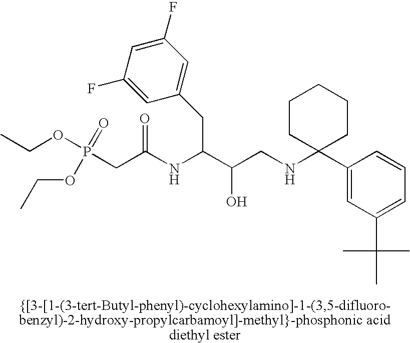 |
| 1-85. | 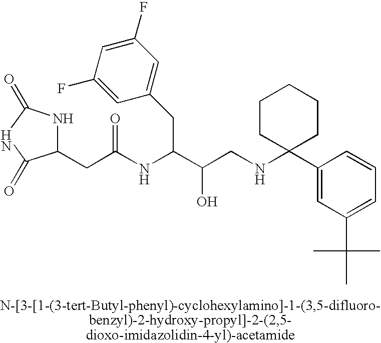 |
| 1-86. | 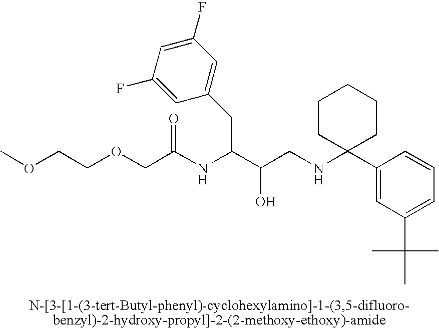 |
| 1-87. | 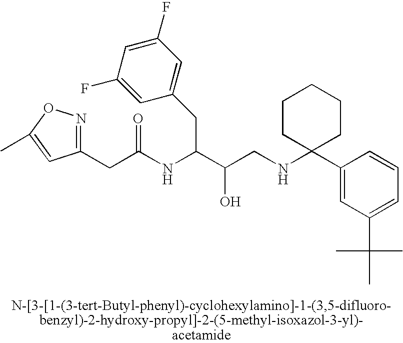 |
| 1-88. | 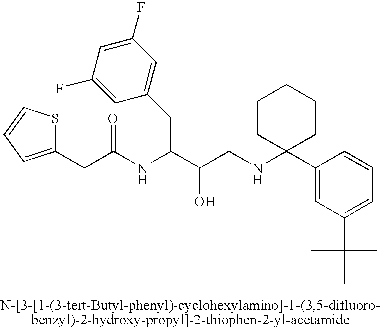 |
| 1-89. | 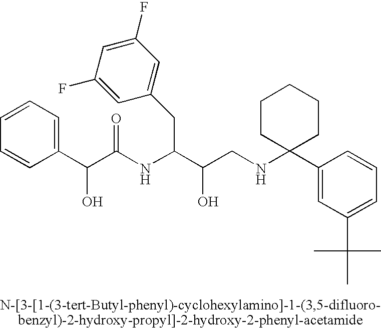 |
| 1-90. | 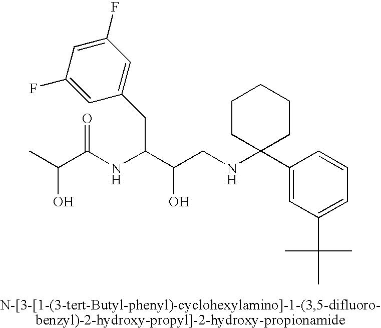 |
| 1-91. | 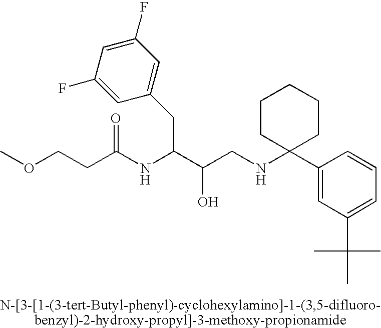 |
| 1-92. | 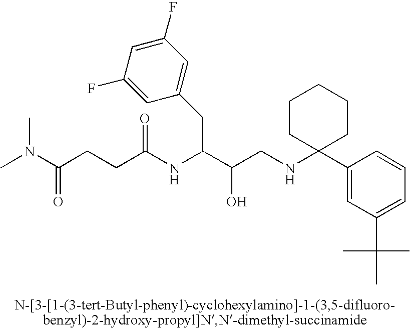 |
| 1-93. | 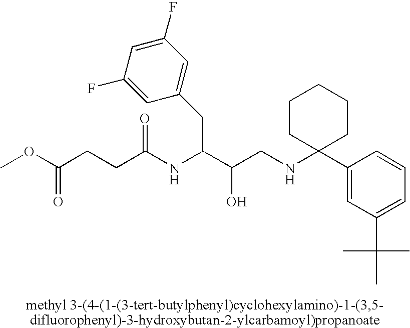 |
| 1-94. | 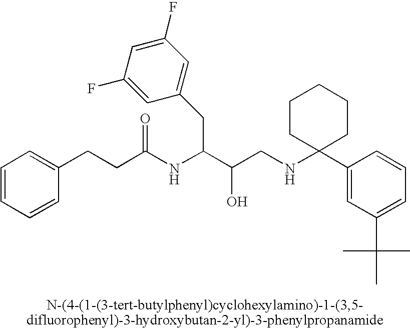 |
| 1-95. | 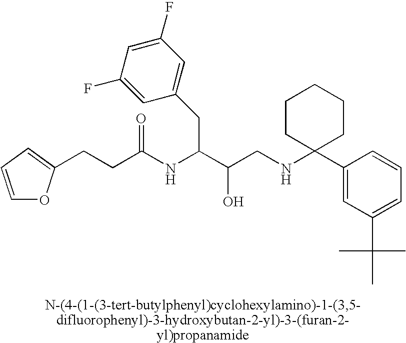 |
| 1-96. | 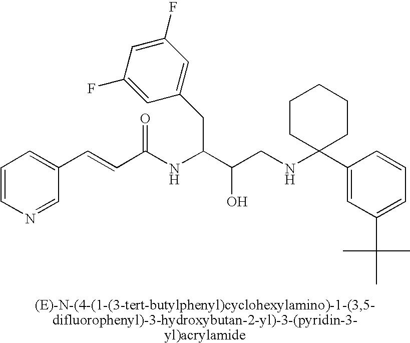 |
| 1-97. | 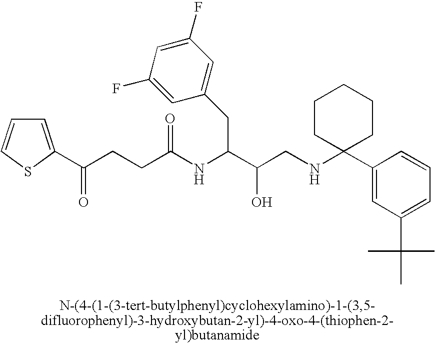 |
| 1-98. | 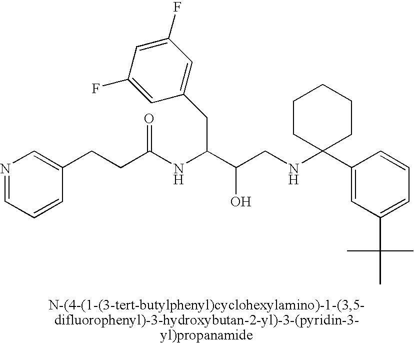 |
| 1-99. | 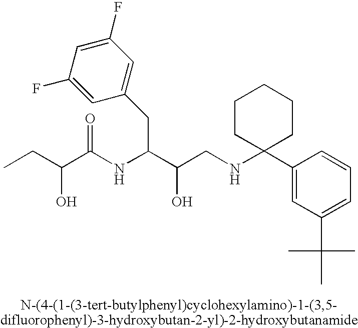 |
| 1-100. | 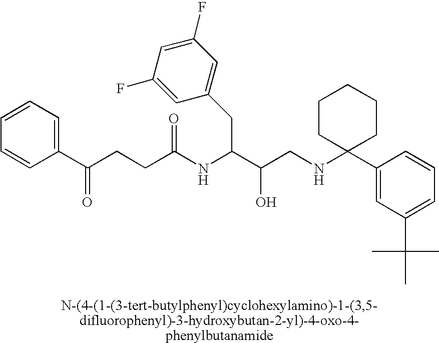 |
| 1-101. | 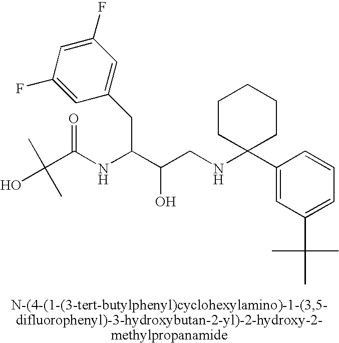 |
| 1-102. | 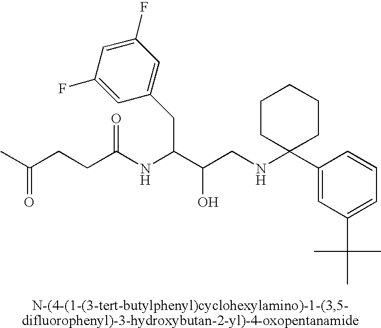 |
| 1-103. | 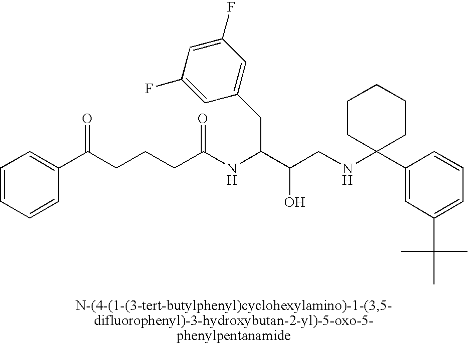 |
| 1-104. | 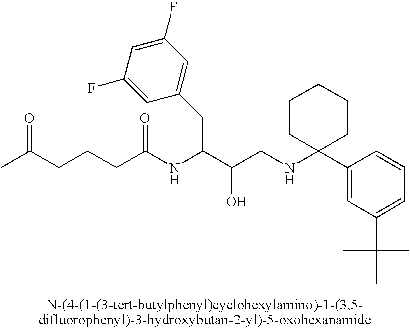 |
| 1-105. | 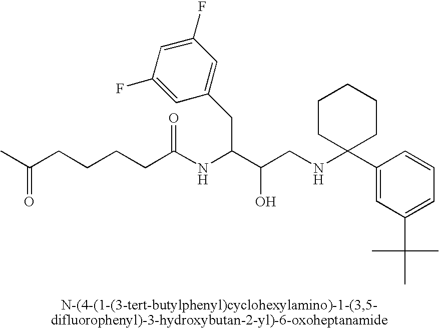 |
| 1-106. | 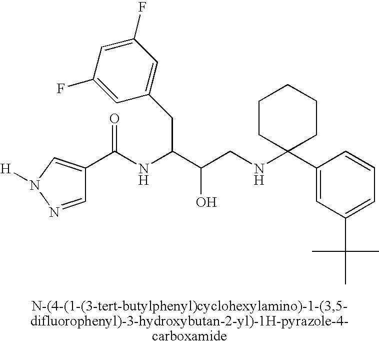 |
| 1-107. | 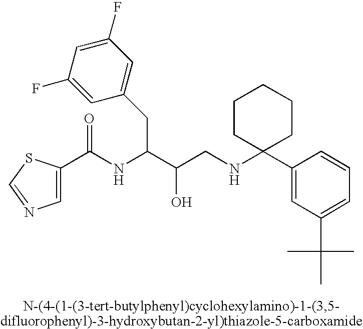 |
| 1-108. | 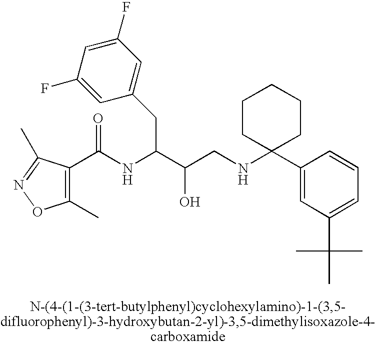 |
| 1-109. | 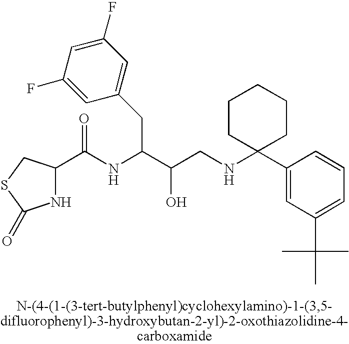 |
| 1-110. | 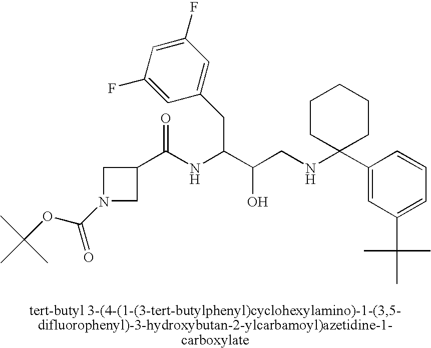 |
| 1-111. | 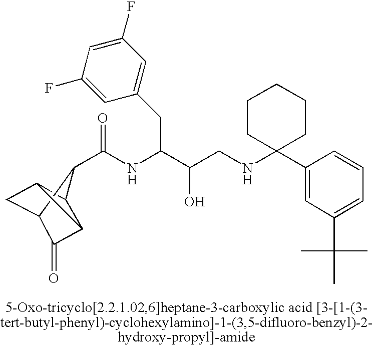 |
| 1-112. | 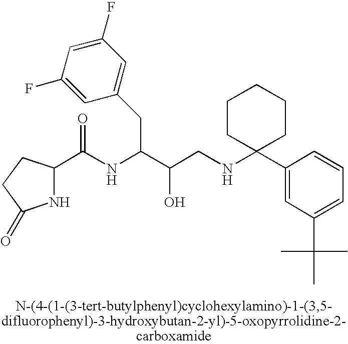 |
| 1-113. | 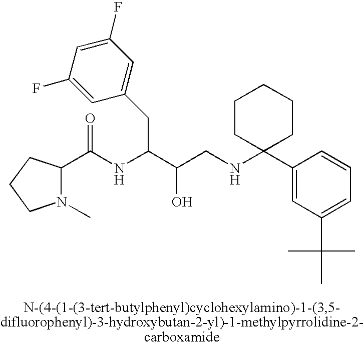 |
| 1-114. | 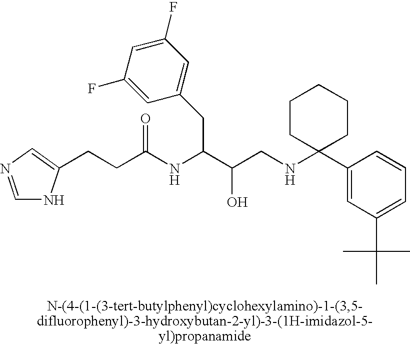 |
| 1-115. | 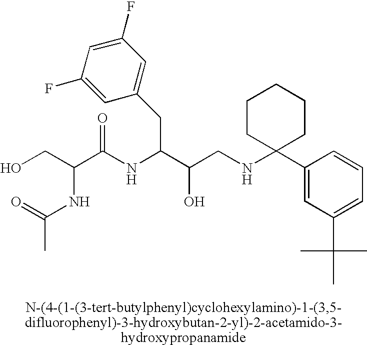 |
| 1-116. | 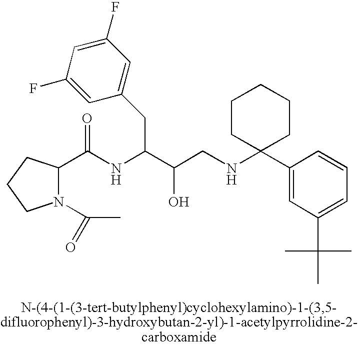 |
| 1-117. | 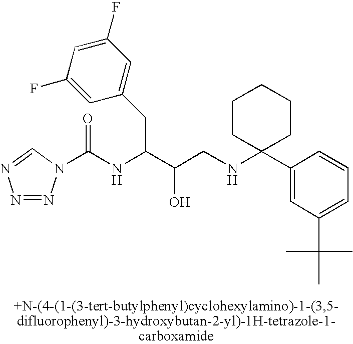 |
| 1-118. | 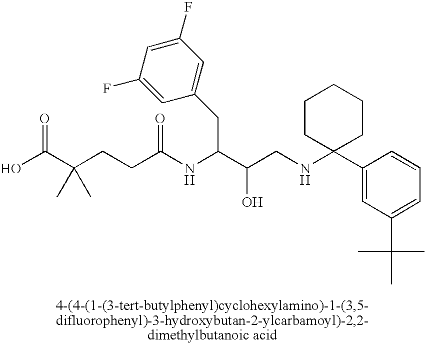 |
| 1-119. | 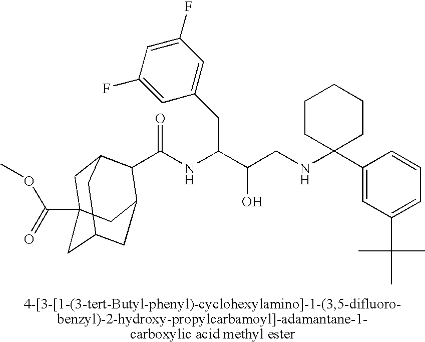 |
| 1-120. | 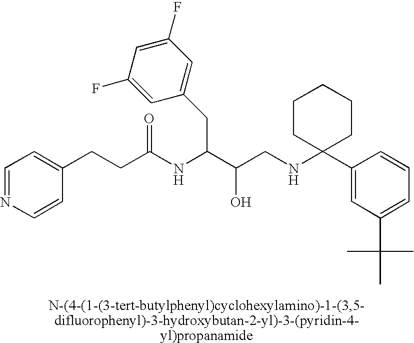 |
| 1-121. | 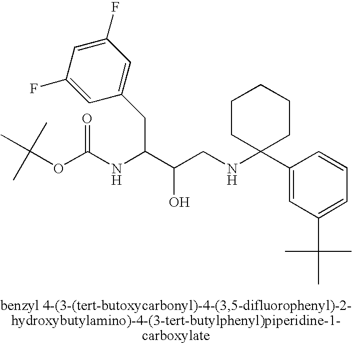 |
| 1-122. | 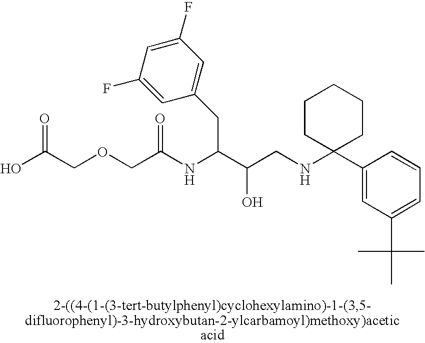 |
| 1-123. | 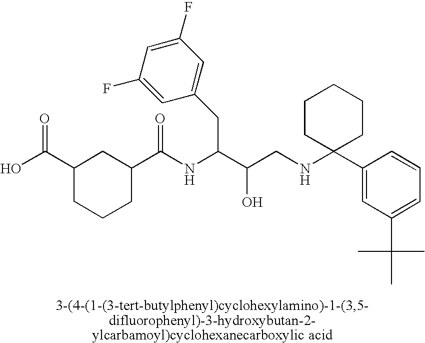 |
| 1-124. | 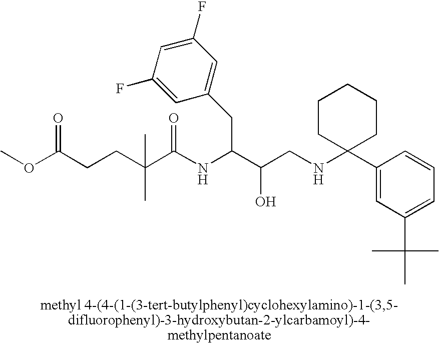 |
| 1-125. | 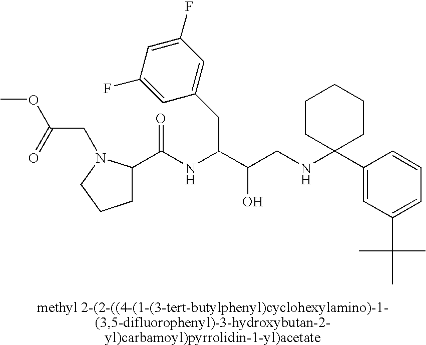 |
| 1-126. | 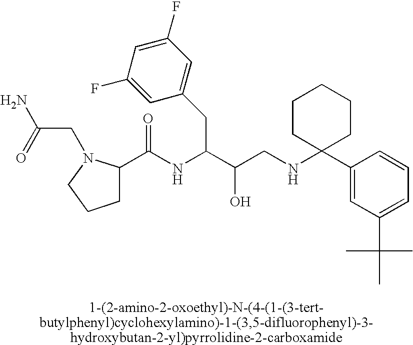 |
| 1-127. | 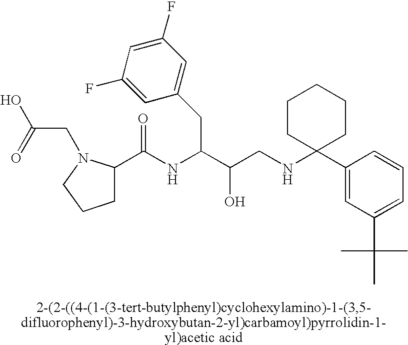 |
| 1-128. | 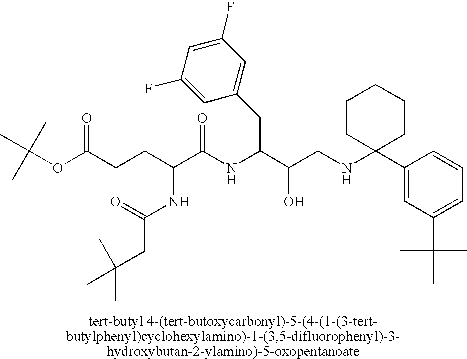 |
| 1-129. | 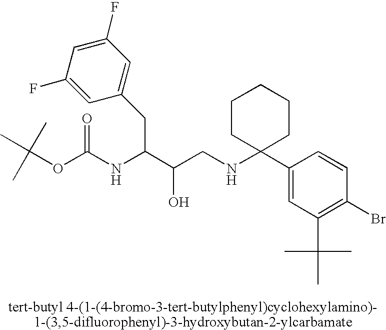 |
| 1-130. | 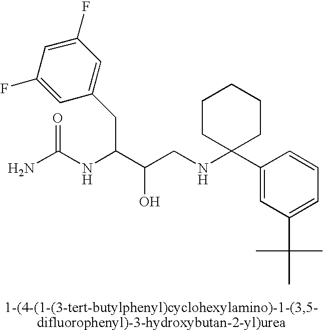 |
| 1-131. | 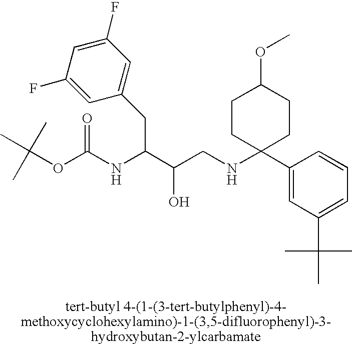 |
| 1-132. | 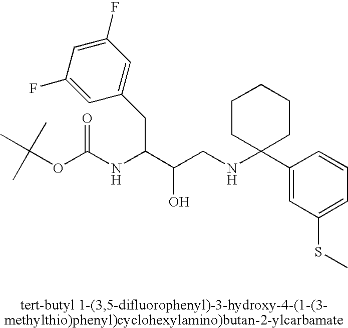 |
| 1-133. | 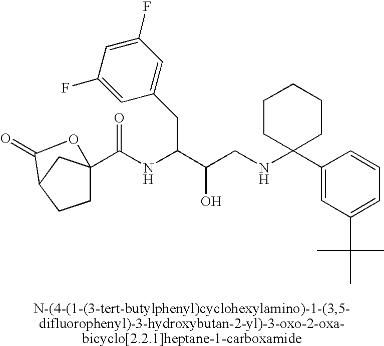 |
| 1-134. | 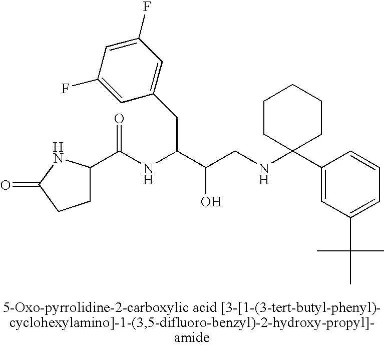 |
| 1-135. | 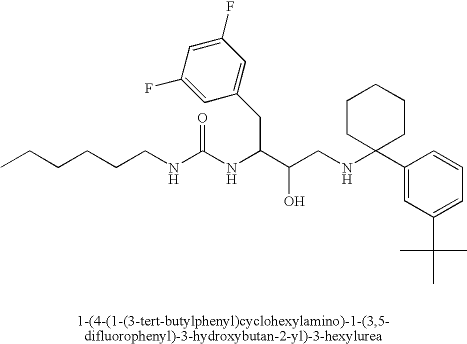 |
| 1-136. | 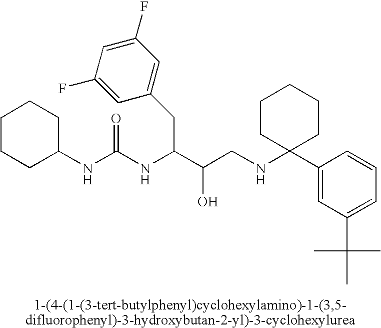 |
| 1-137. | 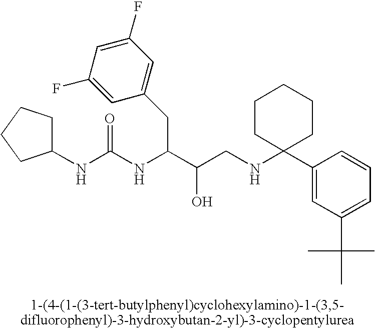 |
| 1-138. | 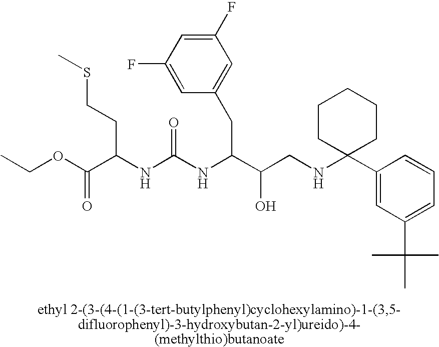 |
| 1-139. | 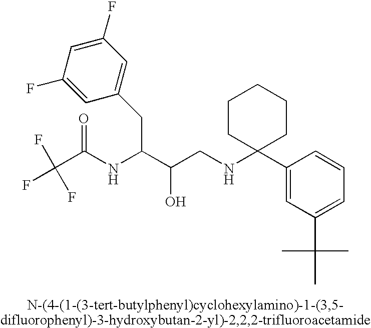 |
| 1-140. | 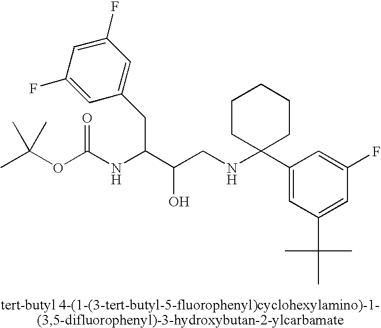 |
| 1-141. | 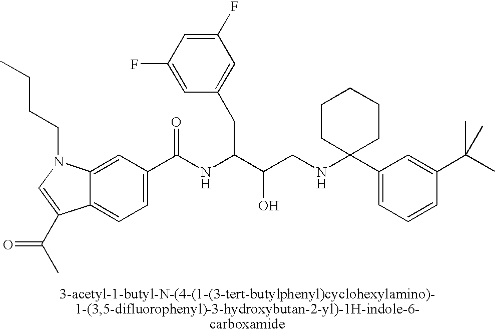 |
| 1-142. | 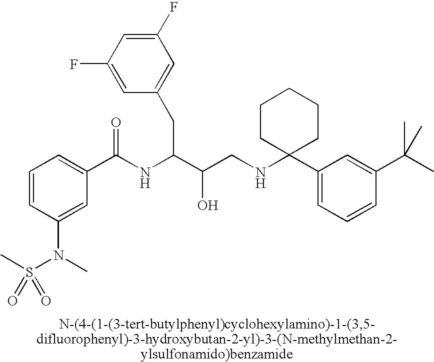 |
| 1-143. | 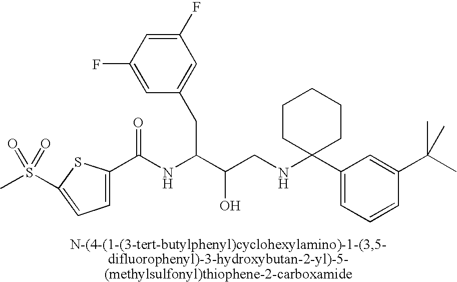 |
| 1-144. | 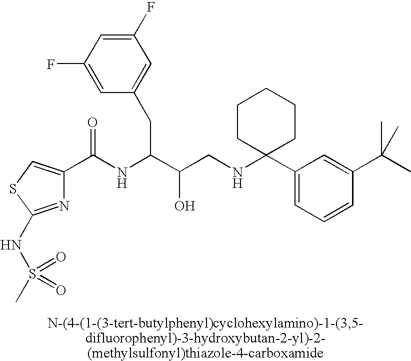 |
| 1-145. | 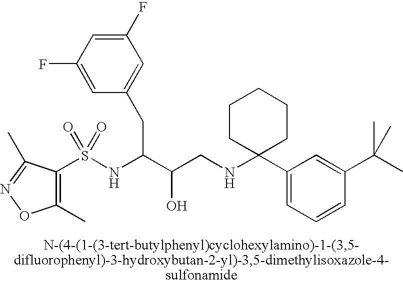 |
| 1-146. | 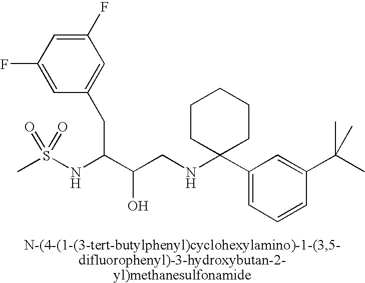 |

### Experimental Procedures

The compounds and methods of treatment of the present invention can be prepared by one skilled in the art based on knowledge of the compound's chemical structure. The chemistry for the preparation of the compounds employed in the methods of treatment of this invention is known to those skilled in the art. In fact, there is more than one process to prepare the compounds employed in the methods of treatment of the present invention. Specific examples of methods of preparation can be found in the art. For examples, see Zuccarello et al., J. Org. Chem. 1998, 63, 4898-4906; Benedetti et al., J. Org. Chem. 1997, 62, 9348-9353; Kang et al., J. Org. Chem. 1996, 61, 5528-5531; Kempf et al., J. Med. Chem. 1993, 36, 320-330; Lee et al., J. Am. Chem. Soc. 1999, 121, 1145-1155; and references cited therein; Chem. Pharm. Bull. (2000), 48(11), 1702-1710; J. Am. Chem. Soc. (1974), 96(8), 2463-72; Ind. J. Chem., §B: Organic Chemistry Including Medicinal Chemistry (2003), 42B(4), 910-915; and J. Chem. Soc. §C: Organic (1971), (9), 1658-10. See also U.S. Pat. Nos. 6,150,530, 5,892,052, 5,696,270, and 5,362,912, and references cited therein, which are incorporated herein by reference.

### EXAMPLE 2

### ¹H, ¹³C NMR, AND MASS SPEC PROCEDURES

¹H and ¹³C NMR spectra were obtained on a Varian 400 MHz, Varian 300 MHz, or Bruker 300 MHz instrument. Mass spec samples analyses were performed with electron spray ionization (ESI).

### EXAMPLE 3

### EXEMPLARY HPLC PROCEDURES

Various High Pressure Liquid Chromatography (HPLC) procedures employed the following methods:

Method [1] utilizes a 20% [B]: 80% [A] to 70% [B]: 30% [A] gradient in 1.7 min, then hold, at 2 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [2] utilizes a 50% [B]: 50% [A] to 95% [B]: 5% [A] gradient in 2.5 min, then hold, at 2 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [3] utilizes a 5% [B]: 95% [A] to 20% [B]: 80% [A] gradient in 2.5 min, then hold, at 2 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [4] utilizes a 20% [B]: 80% [A] to 70% [B]: 30% [A] gradient in 2.3 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [5] utilizes a 50% [B]: 50% [A] to 95% [B]: 5% [A] gradient in 3.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [6] utilizes a 5% [B]: 95% [A] to 20% [B]: 80% [A] gradient in 3.33 min, then hold, at 1.5 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [7] utilizes a 20% [B]: 80% [A] to 70% [B]: 30% [A] gradient in 1.7 min, then hold, at 2 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

Method [8] utilizes a YMC ODS-AQ S-3 120 A 3.0×50 mm cartridge, with a standard gradient from 5% acetonitrile containing 0.01% heptafluorobutyric acid (HFBA) and 1% isopropanol in water containing 0.01% HFBA to 95% acetonitrile containing 0.01% HFBA and 1% isopropanol in water containing 0.01% HFBA over 5 min.

### EXAMPLE 4

### PREPARATION OF PRECURSOR FOR FORMULA (I) COMPOUNDS

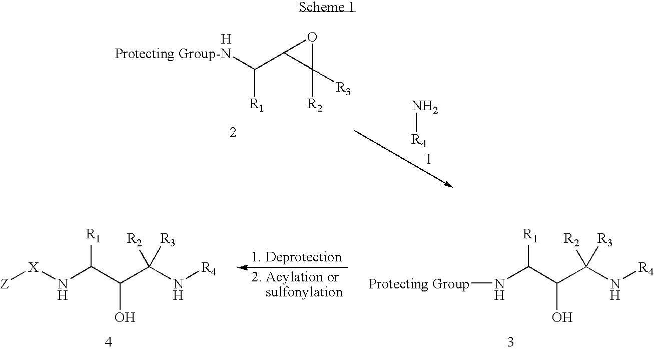

As described above and below, one embodiment of the present invention provides for compounds 4 as shown above in Scheme 1. These compounds may be made by methods known to those skilled in the art from starting compounds that are also known to those skilled in the art. A suitable process for the preparation of compounds 4 is set forth in Scheme 1 above.

The amine 1 is used to open the epoxide 2 yielding the protected amino alcohol 3. Suitable reaction conditions for opening the epoxide 2 include running the reaction in a wide range of common and inert solvents. C₁-C₆ alcohol solvents are preferred, especially isopropyl alcohol. The reactions can be run at temperatures ranging from about 20-25° C. up to about the reflux temperature of the alcohol employed. The preferred temperature range for conducting the reaction is between 50° C. and the refluxing temperature of the alcohol employed.

The protected amino alcohol 3 is deprotected to the corresponding amine by means known to those skilled in the art for removal of amine protecting groups. Suitable means for removal of the amine protecting group depend on the nature of the protecting group. Those skilled in the art, knowing the nature of a specific protecting group, know which reagent is preferable for its removal. For example, it is preferred to remove the preferred protecting group, Boc, by dissolving the protected 3 in a trifluoroacetic acid/dichloromethane (1/1) mixture. When complete, the solvents are removed under reduced pressure yielding the corresponding amine (as the corresponding salt, i.e. trifluoroacetic acid salt) which is used without further purification. If desired, the amine can be purified further by means well known to those skilled in the art, such as, for example, recrystallization. Further, if the non-salt form is desired, it also can be obtained by means known to those skilled in the art, such as, for example, preparing the free base amine via treatment of the salt with mild basic conditions. Additional Boc deprotection conditions and deprotection conditions for other protecting groups can be found in T. W. Green and P. G. M. Wuts in Protecting Groups in Organic Chemistry, 3^{rd} edition, John Wiley and Sons, 1999.

The amine is then reacted with an appropriately substituted amide forming agent Z-(CO)—Y to produce coupled amides 4 by nitrogen acylation means known to those skilled in the art. Nitrogen acylation conditions for the reaction of amine with an amide forming agent Z-(CO)—Y are known to those skilled in the art and can be found in, for example, R. C. Larock in Comprehensive Organic Transformations, VCH Publishers, 1989, p. 981, 979, and 972. Y comprises —OH (carboxylic acid) or halide (acyl halide), preferably chlorine, imidazole (acyl imidazole), or a suitable group to produce a mixed anhydride.

### EXAMPLE 5

### ALTERNATIVE PREPARATION OF PRECURSORS FOR FORMULA (I) COMPOUNDS

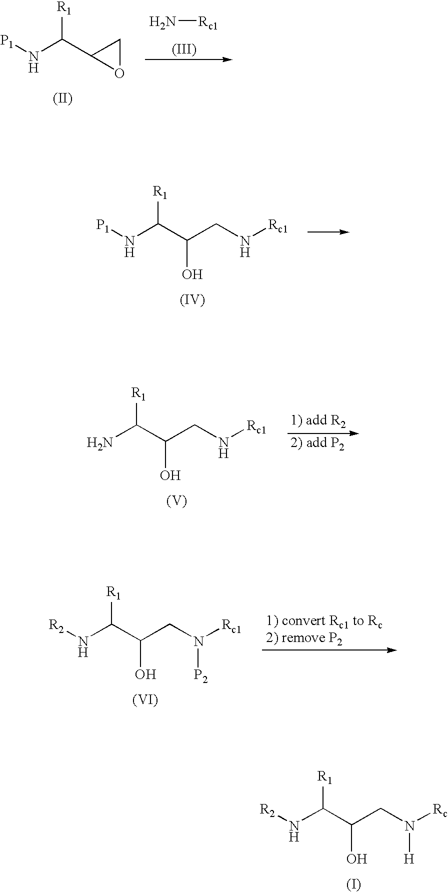

An alternative approach for converting a reactive group to yield compounds (I) utilizes a common advanced intermediate (VI). Epoxides (II) are treated with 1.5-5 equivalents of primary amine H₂N-R_{c1} (III) in an alcoholic solvent, such as ethanol, isopropanol, or sec-butanol to effect ring opening of the epoxide. In an embodiment, this reaction is prepared at elevated temperatures from 40° C. to reflux. In another embodiment, this reaction is performed at reflux in isopropanol. The resulting amino alcohol (IV) is then deprotected.

When R_{c1} contains a labile functional group, such as an aryl iodide, aryl bromide, aryl trifluoromethanesulfonate, or aryl boronic ester, which may be converted into R_{C} via transition metal-mediated coupling, it is possible to rapidly synthesize a variety of analogs (I). Such conversions may include, for example, Suzuki (aryl boronic acid or boronic ester and aryl halide), Negishi (arylzinc and aryl or vinyl halide), and Sonogashira (arylzinc and alkynyl halide) couplings. Subsequent to the coupling reaction, the protecting group P₂ is removed in methods known in the art to yield compounds (I).

### EXAMPLE 6

### PREPARATION OF PRECURSOR SUBSTITUTED AMINES

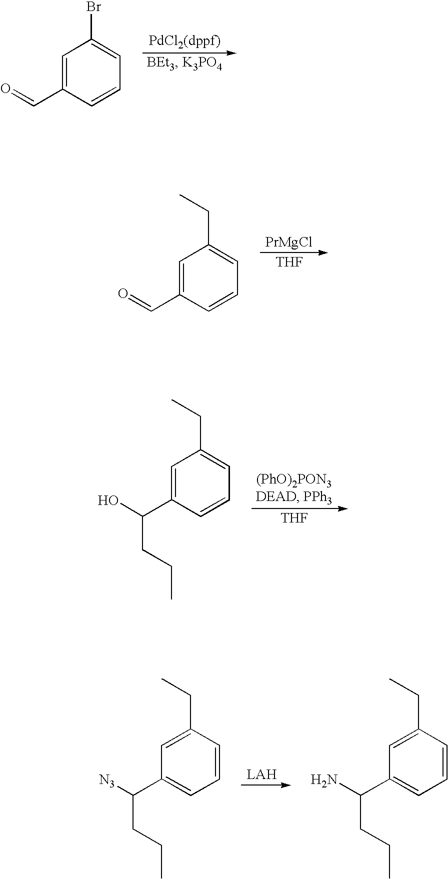

Precursor amines can generally be prepared as shown above. Specific examples are described below.

### EXAMPLE 7

### PREPARATION OF FLUOROACETYL IMIDAZOLE

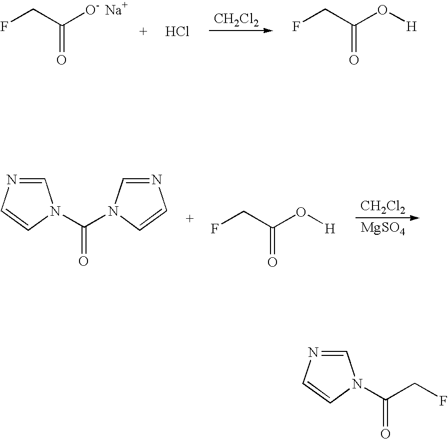

To a slurry of 1.2 g (12 mmol) of sodium fluoroacetate in 25 mL of CH₂Cl₂ is added, with swirling of the flask, 1 mL (12 mmol) of concentrated HCl. About 1 teaspoonful of anhydrous magnisium sulfate is added to the flask, and the contents are filtered, rinsing the filter paper with 15 mL of CH₂Cl₂. The combined filtrate and wash are placed under N₂(g), and 1.3 g (8 mmol) of carbonyldiimidazole is added portion-wise to the stirring mixture over 20 min. NMR analysis of an aliquot removed 40 min later indicated that the reaction was nearly complete. After 1 h a teaspoonful of magnisium sulfate is added, and the mixture is allowed to stir overnight. It is filtered and concentrated to remove most of the CH₂Cl₂, leaving 1.6 g of a pale yellow oil. The NMR spectrum indicates the presence of CH₂Cl₂, fluoroacetic acid, imidazole, and fluoroacetyl imidazole: ¹H NMR (CDCl₃); δ 8.26 (s, 1 H), 7.53 (s, 1 H), 7.15 (s, 1 H), 5.40 (d, J=47 Hz, 2 H). Integration reveals the oil to be 28% by weight fluoroacetyl imidazole (0.45 g, 3.5 mmol, 44%). The oil is diluted with CH₂Cl₂ to make a solution that is 0.2 M fluoroacetyl imidazole.

### EXAMPLE 8

### SYNTHESIS OF PYRIDINE DERIVATIVES

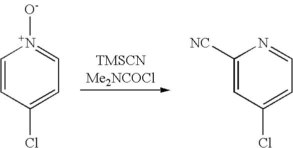

The nitrile was introduced essentially according to the method of Omstein, P. L. et al. J. Med. Chem., 1991, 34, 90-97. The crude product was filtered through silica (CH₂Cl₂ elution) yielding the product as a white crystalline solid. ¹H NMR (300 MHz, CDCl₃); 8 8.64 (d, J=5.3 Hz, 1 H), 7.72 (d, J=1.7 Hz, 1 H), 7.56 (dd, J=5.3, 1.7 Hz, 1H); MH+ (Cl): 139.0 (³⁵Cl).

### EXAMPLE 9

### PREPARATION OF 2-CYANO-4-ISOPROPYLPYRIDINE

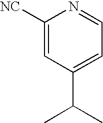

2-Cyano-4-isopropylpyridine was synthesized according to the method of Ornstein, P. L. et al. J. Med. Chem., 1991, 34, 90-97: MH+ (Cl): 147.1.

### EXAMPLE 10

### PREPARATION OF 2-CYANO-4-TERT-BUTYLPYRIDINE

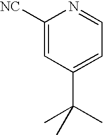

2-Cyano-4-tert-butylpyridine was synthesized according to the method of Ornstein, P. L. et al. J. Med. Chem., 1991, 34, 90-97: ¹H NMR (300 MHz, CDCl₃); δ 8.60 (d, J=5.3 Hz, 1H), 7.68 (d, J=1.5 Hz, 1H), 7.49 (dd, J=5.3,1.9 Hz, 1 H), 1.33 (s, 9H); MH+ (Cl): 161.1.

### EXAMPLE 11

### PREPARATION OF 2-CYANO-6-NEOPENTYLPYRIDINE

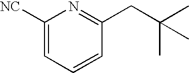

2-Cyano-6-neopentylpyridine was synthesized from 2-neopentylpyridine according to the method of Ornstein, P. L. et al. J. Med. Chem., 1991, 34, 90-97: R_{f}=0.62 in 20% EtOAc/hexanes; MH+ (Cl): 175.1.

### EXAMPLE 12

### PREPARATION OF 2-NEOPENTYLPYRIDINE FROM 2-BROMOPYRIDINE

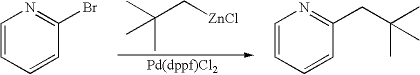

A solution of neopentylzinc chloride was prepared according to the method of Negishi, E.-I. et al. Tetrahedron Lett., 1983, 24, 3823-3824.

2-Bromopyridine (Aldrich, 0.48 mL, 5.0 mmol) and [1,1′-bis(diphenylphosphino)ferrocene]dichloropalladium(II), complex with dichloromethane (1:1) (Aldrich, 200 mg, 0.25 mmol) were added to the neopentylzinc chloride suspension. The resulting suspension was stirred at room temperature for 21 h, whereupon saturated ammonium chloride solution (25 mL) was added. The mixture was extracted with ethyl acetate (3×). The combined organic extracts were dried (sodium sulfate), filtered and concentrated under reduced pressure. The residue was dissolved in methylene chloride, and washed with 1 N HCl. The aqueous layer was separated, basified with 10 N NaOH (aq), and extracted with CH₂Cl₂. The organic layer was dried (sodium sulfate), filtered and concentrated under reduced pressure yielding 2-neopentylpyridine as an oil: R_{f}=0.33 in 5% MeOH/CH₂Cl₂.

### EXAMPLE 13

### PREPARATION OF 2-CYANO-4-NEOPENTYLPYRIDINE

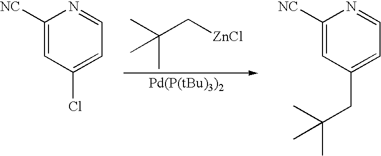

This transformation was performed according to the method of Dai, C. and Fu, G. J. Am. Chem. Soc., 2001, 123, 2719-2724. The crude residue was purified by filtration through a small plug of silica (20% ether/hexanes elution) yielding 2-cyano-4-neopentylpyridine: R_{f}=0.25 in 20% Et₂O/hexanes; MH+ (CI): 175.1.

### EXAMPLE 14

### PREPARATION OF 4-CYANO-2-NEOPENTYLPYRIDINE

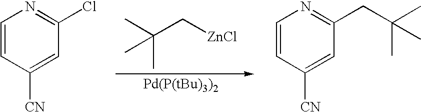

The method for the synthesis of 2-cyano-4-neopentylpyridine described in Example 13 was used to convert 2-chloro-4-cyanopyridine (Oakwood) into 4-cyano-2-neopentylpyridine: R_{f}=0.47 in 10% EtOAc/hexanes; ¹H NMR (300 MHz, CDCl₃); δ 8.73 (dd, J=4.9, 0.7 Hz, 1H), 7.55-7.40 (m, 2H), 2.75 (s, 2H), 0.96 (s, 9H); MH+ (Cl): 175.1

### EXAMPLE 15

### PREPARATION OF 5-BROMO-2-(1H-IMIDAZOL-1-YL)BENZONITRILE

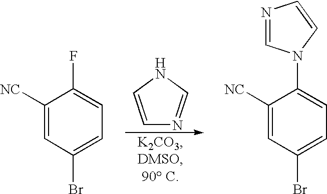

K₂CO₃ (3.337 g, 24.4 mmol) was added to a stirred solution of 5-Bromo-2-fluorobenzonitrile (2.5 g, 12.2 mmol) in DMSO (50 mL), followed by the addition of 1H-imidazole (996 mg, 14.64 mmol). The reaction mixture was heated to 90° C. overnight, and diluted with water. The reaction mixture was extracted with EtOAC (×2). The organic layer was washed with water and brine, dried with sodium sulfate, filtered, and concentrated under reduced pressure to yield 2.97 g of the imidazolylbenzonitrile as an off-white solid (98% yield). ¹H NMR (CDCl₃); δ 7.97 (m, 2 H), 7.90 (m, 1 H), 7.41 (d, J=8 Hz, 1 H), 7.37 (s, 1 H), 7.32 (s, 1 H).

### EXAMPLE 16

### PREPARATION OF 5-(2,2-DIMETHYLPROPYL)-2-IMIDAZOL-1-YL-BENZONITRILE

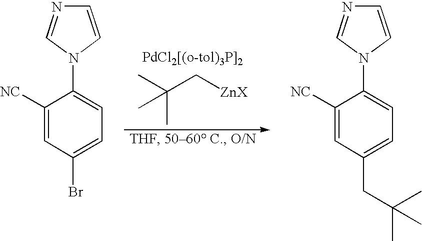

Neopentyl iodide (25.4 mL, 191 mmol) was added to a Rieke Zn suspension (250 mL, 191 mmol, 5 g/100 mL THF from Aldrich) placed in a 1 L flask at room temperature. It was then heated to 50° C. for 3 h. Dichlorobis(tri-o-tolylphosphine)palladium(II) (5.0 g, 6.4 mmol) and 5-bromo-2-(1H-imidazol-1-yl)benzonitrile (16 g, 64.5 mmol) were added in portions to the stirring suspension at 50° C. The reaction mixture was heated at 50-60° C. for 17 h.

The reaction was quenched by addition of 100 mL 1 N HCl, then filtered through celite, and separated. The organic layer was washed with water (100 mL), followed by 4×100 mL 1 N HCl. The acidic extracts were combined, and basified with 10 N NaOH to pH 12. The resulting aqueous suspension was extracted with 3×200 mL EtOAc. The combined extracts were washed with 100 mL brine, dried (sodium sulfate), filtered, and concentrated in vacuo. TLC (50-50% EtOAc/hex) indicated nearly pure desired product with a small amount of baseline material. The crude material (7 g) was taken to subsequent reaction without further purification: MH+ 240.1.

### EXAMPLE 17

### PREPARATION OF 2-(1H-IMIDAZOL-1-YL)-5-ISOBUTYL BENZONITRILE

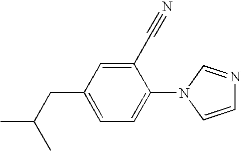

The above compound was prepared essentially according to the method of Example 15, but the reaction mixture was only stirred overnight. The resulting crude product was purified by flash column chromatography (50-100% ethyl acetate: hexane) yielding the product as a dark-brown oil. ¹H NMR (CDCl₃); δ 7.89 (s, 1 H), 7.60 (s, 1 H), 7.53 (d, J=8 Hz, 1 H), 7.40 (m, 2 H), 7.28 (m, 1 H), 2.60 (d, J=8 Hz, 2 H), 1.93 (m, 1 H), 0.97 (d, 6 H); ESI-MS [M+H⁺]+=226.03.

### EXAMPLE 18

### PREPARATION OF 1-(3-ETHYL-PHENYL)-CYCLOHEXANOL FROM 1-BROMO-3-ETHYLBENZENE

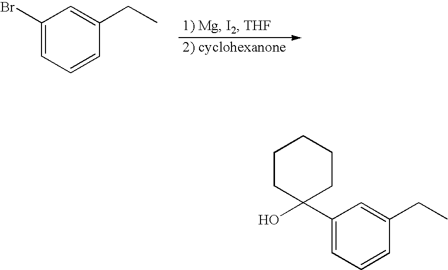

Magnesium turnings (1.35 g, 55.53 mmol) were activated via vigorous stirring overnight under N₂ (g) inlet. A few crystals of iodine were added to the flask, which was then flame-dried under vacuum. Anhydrous THF (3 mL) was added to the reaction flask followed by 1-bromo-3-ethylbenzene (Avocado Chemicals, 2.0 mL, 14.59 mmol). The reaction was initiated after briefly heating with a heat gun. To this was added the remainder of 1-bromo-3-ethylbenzene (1.7 mL, 12.43 mmol) in a THF solution (15 mL). The reaction mixture was refluxed for 2 h. A cyclohexanone (2.2 mL, 21.22 mmol) in THF (8 mL) solution was added once the flask was cooled to 0° C. After 3.5 h the reaction mixture was quenched with H₂O over an ice bath and partitioned between Et₂O and H₂O. The organic layer was removed and acidified with 1 N HCl. The organic layer was separated, dried (sodium sulfate), and concentrated under reduced pressure. The residue was purified by flash chromatography (100% CHCl₃) yielding the desired alcohol (4.152 g, 96%): mass spec (Cl) 187.1 (M-16).

### EXAMPLE 19

### PREPARATION OF 1-(1-AZIDO-CYCLOHEXYL)-3-ETHYL-BENZENE FROM 1-(3-ETHYL-PHENYL)-CYCLOHEXANOL

1-(3-Ethyl-phenyl)-cyclohexanol (4.02 g, 19.68 mmol) in anhydrous chloroform (45 mL) was cooled to 0° C. under N₂ (g) inlet. Sodium azide (3.97 g, 61.07 mmol) was added followed by dropwise addition of trifluoroacetic acid (7.8 mL, 101.25 mmol). The reaction mixture was refluxed for 2 h and allowed to stir at room temperature overnight. This was then partitioned between H₂O and Et₂O. The aqueous layer was removed and the mixture was washed with H₂O followed by 1.ON NH₄OH. The organic layer was separated, dried (sodium sulfate), and concentrated under reduced pressure. The crude product was used without further purification (3.30 g, 73%): mass spec (Cl) 187.1 (M-42).

### EXAMPLE 20

### PREPARATION OF 1-(3-ETHYL-PHENYL)-CYCLOHEXYLAMINE FROM 1-(1-AZIDO-CYCLOHEXYL)-3-ETHYL-BENZENE

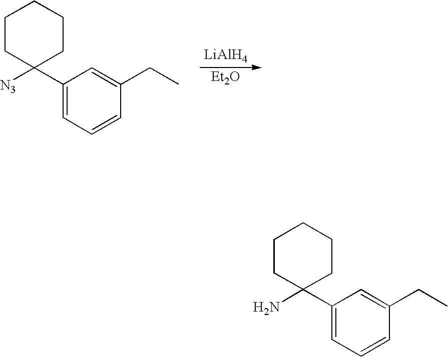

A solution of 1-(1-azido-cyclohexyl)-3-ethyl-benzene (1.94 g, 8.39 mmol) in Et₂O (8 mL) was added dropwise to a suspension of lithium aluminum hydride (0.31 g, 8.17 mmol) in THF (30 mL). This was stirred at room temperature under N₂ (g) inlet for 3 h, whereupon the reaction was quenched with 1.0N NaOH. The reaction mixture was then partitioned between Et₂O and 1 N HCl. The aqueous layer was collected and basified with 2 N NH₄OH and extracted with CHCl₃. The organic layer was separated, dried (sodium sulfate), filtered, and concentrated under reduced pressure. The crude product was used without further purification: mass spec (Cl) 187.1 (M-16).

### EXAMPLE 21

### PREPARATION OF 1-(3-ISOPROPYLPHENYL)CYCLO HEXANAMINE HYDROCHLORIDE

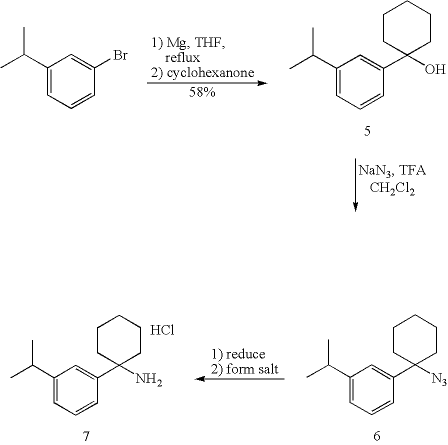

### Step 1. Preparation of 1-(3-isopropylphenyl)cyclohexanol (5)

To 1.2 g (50 mmol) of magnesium turnings in 15 mL of dry THF is added a small crystal of iodine followed by 40 μL of dibromoethane. This mixture is placed in a water bath at 50° C. and 3-isopropylbromobenzene (5.0 g, 25 mmol) in 15 mL of dry tetrahydrofuran (THF) is added dropwise over 20 min, while the bath temperature is raised to 70° C. The mixture is stirred and refluxed for 40 additional min. The solution is cooled in an ice-water bath and cyclohexanone (2.0 mL, 19 mmol) in 10 mL of dry THF is added dropwise over 15 min. The ice bath is removed and the mixture is allowed to warm to ambient temperature over 1 h. The solution is decanted into aqueous saturated NH₄Cl and combined with an ether wash of the residual magnesium turnings. The organic phase is washed twice more with aqueous NH₄Cl, dried over anhydrous sodium sulfate, filtered and concentrated. Chromatography on silica gel, eluting with 10% ethyl acetate in heptane, affords 2.7 g (12 mmol, 60%) of 1-(3-isopropylphenyl)cyclohexanol 5 as an oil: ¹H NMR (CDCl₃); δ 7.39 (m, 1 H), 7.3 (m, 2 H), 7.12 (m, 1 H), 2.92 (m, 1 H), 1.84-1.54 (m, 10 H), 1.26 (d, J=7 Hz, 6 H).

### Step 2. Preparation of 1-(3-isopropylphenyl)cyclohexylazide (6)

To 3.20 g (14.7 mmol) of 1-(3-isopropylphenyl)cyclohexanol 5 in 60 mL of CH₂Cl₂ under nitrogen is added 2.10 g (32.3 mmol) of sodium azide. The stirred suspension is cooled to −5° C. and a solution of trifluoroacetic acid (9.0 mL, 120 mmol) in 35 mL of dichloromethane is added dropwise over 1 h. The resulting suspension is stirred at 0° C. for an additional hour. 10 mL of water is added dropwise to the cold, vigorously stirred mixture, followed by dropwise addition of a mixture of 10 mL of water and 10 mL of concentrated ammonium hydroxide. After 30 min the mixture is poured into a separatory funnel containing 350 mL of a 1:1 mixture of heptane and ethyl acetate, and 100 mL of water. The organic phase is washed with an additional portion of water, followed successively by 1 N KH₂PO₄, water, and brine. It is then dried over anhydrous sodium sulfate, filtered and concentrated yielding 3.6 g (14.7 mmol, 100%) of 6 as a pale yellow oil: ¹H NMR (CDCl₃); δ 7.3 (m, 2 H), 7.25 (m, 1 H), 7.16 (m, 1 H), 2.92 (m, 1 H), 2.01 (m, 2 H), 1.83 (m, 2 H), 1.73-1.64 (m, 5 H), 1.3 (m, 1 H), 1.26 (d, J=7 Hz, 6 H).

### Step 3. Preparation of 1-(3-isopropylphenyl)cyclohexanamine hydrochloride (7)

To 1-(3-isopropylphenyl)cyclohexylazide 6 (2.7 g, 11 mmol) in 200 mL of ethanol is added 20 mL of glacial acetic acid and 0.54 g of 10% palladium on carbon. The mixture is evacuated and placed under 16 psi of hydrogen, with shaking, for 2.5 h. The reaction mixture is filtered, the catalyst is washed with ethanol, and the solvents are removed in vacuo. Residual acetic acid is removed by chasing the residue with toluene. The acetate salt is dissolved in ethyl acetate and 1 N NaOH is added. The organic phase is washed with more 1 N NaOH and then with water, dried over sodium sulfate, filtered and concentrated. The residue is dissolved in ether and ethereal HCl (concentrated HCl in ether that has been stored over magnisium sulfate) is added yielding a white solid. This is filtered, washed with ether, collected as a solution in dichloromethane, and concentrated yielding 2.1 g (8.3 mmol, 75%) of hydrochloride 7 as a white solid. ¹H NMR (CDCl₃); 8 8.42 (br s, 3 H), 7.43 (m, 2 H), 7.25 (m, 1 H), 7.15 (m, 1 H), 2.92 (hept, J=7 Hz, 1 H), 2.26 (m, 2 H), 2.00 (m, 2 H), 1.69 (m, 2 H), 1.45-1.3 (m, 4 H), 1.24 (d, J=7 Hz, 6 H); IR (diffuse reflectance); 2944, 2864, 2766, 2707, 2490, 2447, 2411, 2368, 2052, 1599, 1522, 1455, 1357, 796, 704 cm⁻¹. MS (EI)m/z(relative intensity) 217 (M+,26), 200 (13), 175 (18), 174 (99), 157 (15), 146 (23), 132 (56), 131 (11), 130 (16), 129 (18). HRMS (ESI) calculated for C₁₅H₂₃N+H₁ 218.1909, found 218.1910. Anal. Calculated for C₁₅H₂₃N.HCl: C, 70.98; H, 9.53; N, 5.52; Cl, 13.97. Found: C, 70.98; H, 9.38; N, 5.49.

### EXAMPLE 22

### PREPARATION OF N-((1S,2R)-1-(3,5-DIFLUOROBENZYL)-2-HYDROXY-3-([1-(3-ISOPROPYLPHENYL)CYCLOHEXYL]AMINO}PROPYL)ACETAMIDE HYDROCHLORIDE

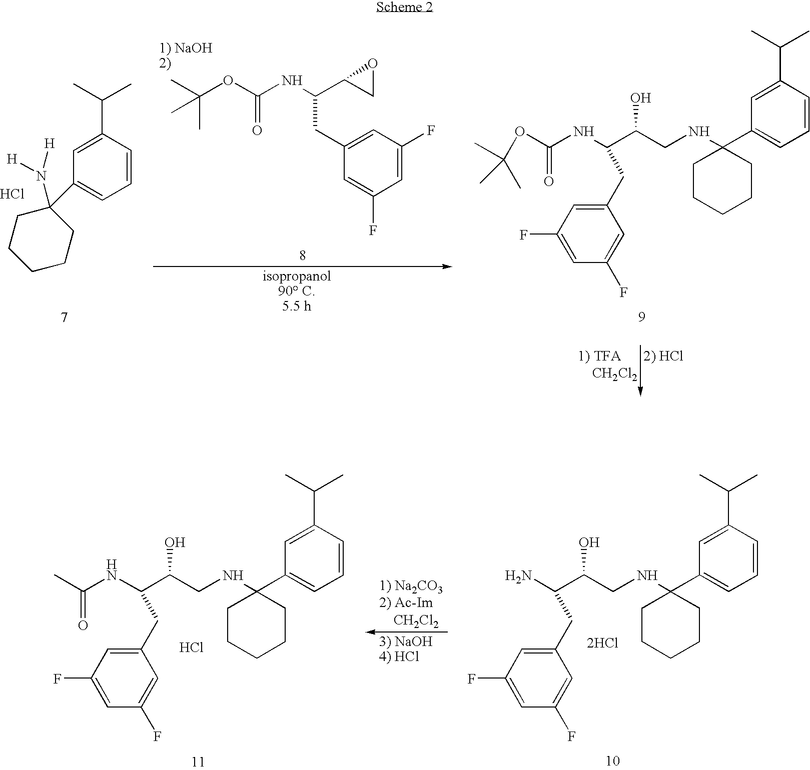

### Step 1. Preparation of tert-butyl (1S,2R)-1-(3,5-difluorobenzyl)-2-hydroxy-3-{[1-(3-isopropylphenyl)cyclohexyl]amino}propylcarbamate (9)

1-(3-isopropylphenyl)cyclohexanamine hydrochloride 7 (2.1 g, 8.3 mmol) is shaken with aqueous 1 N NaOH and ethyl acetate. The layers are separated and the organic phase is washed sequentially with aqueous NaOH and then with 1 N NaHCO₃. The organic layer is then dried over sodium sulfate, filtered, and concentrated yielding a quantitative yield (1.8 g) of the free amine as an oil. [2-(3,5-Difluoro-phenyl)-1-oxiranyl-ethyl]-carbamic acid tert-butyl ester (8, 1.5 g, 5.0 mmol) is combined with the free amine in 35 mL of isopropyl alcohol, and the mixture is heated at reflux for 5.5 h, under nitrogen. The mixture is cooled and concentrated in vacuo. The resulting residue is dissolved in 250 mL of ethyl ether, which is washed four times with 30 mL portions of aqueous 10% HCl to remove much of the excess amine 7. The ether phase is then washed twice with 1 N NaHCO₃, once with brine, dried over sodium sulfate, filtered, and concentrated. The concentrate is chromatographed over silica gel, eluting with 4% to 6% methanol (containing 2% NH₄OH) in CH₂Cl₂ yielding 1.98 g (3.8 mmol, 77%) of 9 as a viscous oil: ¹H NMR (CDCl₃); δ 7.28-7.21 (m, 3 H), 7.09 (m, 1 H), 6.69 (m, 2 H), 6.62 (m, 1 H), 4.68 (d, J=10 Hz, 1 H), 3.74 (m, 1 H), 3.47 (m, 1 H), 2.93-2.86 (m, 2 H), 2.67 (dd, J=8, 14 Hz, 1 H), 2.32 (m, 2 H), 1.88 (m, 4 H), 1.63-1.52 (m, 5 H), 1.36 (s+m, 10 H), 1.24 (d, J=7 Hz, 6 H); MS (Cl) m/z 517.4 (MH+).

### Step 2. Preparation of (2R,3S)-3-amino-4-(3,5-difluorophenyl)-1-{[1-(3-isopropylphenyl)cyclohexyl]amino}butan-2-ol dihydrochloride (10)

To 1.98 g (3.8 mmol) of tert-butyl (1S,2R)-1-(3,5-difluorobenzyl)-2-hydroxy-3-{[1-(3-isopropylphenyl)cyclohexyl]amino}propylcarbamate 9 in 15 mL of CH₂Cl₂ is added 6.5 mL of trifluoroacetic acid. The mixture is stirred under a nitrogen atmosphere for 1 h and then concentrated. The resulting residue is taken up in ethyl acetate and washed twice with 10% Na₂CO₃ and once with 1 N NaHCO₃. The organic layer is dried over anhydrous sodium sulfate, filtered, and concentrated yielding 1.6 g (quant.) of a pale yellow oil (free base of 10), which is generally carried on in the next step without characterization. The yellow oil may be dissolved in ether and treated with ethereal HCl to precipitate dihydrochloride 10 as a white solid after trituration with ether: ¹H NMR (CDCl₃+CD₃OD drop); δ 7.55 (s, 1 H), 7.45-7.15 (m, 3 H), 6.85 (m, 2 H), 6.75 (m, 1 H), 4.4 (d, J=9.5 Hz, 1 H), 3.82 (m, 1 H), 2.97 (m, 2 H), 2.81 (dd, J=8, 14 Hz, 1 H), 2.65 (m, 2 H), 2.5 (obscured by water) 2.26 (m, 1 H), 2.13 (m, 2 H), 1.79 (m, 2 H), 1.59 (m, 1 H), 1.45-1.25 (m, 3 H), 1.28 (d, J=7 Hz, 6 H); MS (Cl) m/z 417.3 (MH+).

### Step 3. Preparation of N-((1S,2R)-1-(3,5-difluorobenzyl)-2-hydroxy-3-{[1-(3-isopropylphenyl)cyclohexyl]amino}propyl)acetamide hydrochloride (11)

The free base of (2R,3S)-3-amino-4-(3,5-difluorophenyl)-1-{[1-(3-isopropylphenyl)cyclohexyl]amino}butan-2-ol dihydrochloride 10 (1.6 g, 3.8 mmol) is dissolved in 20 mL of CH₂Cl₂ under nitrogen, and 0.87 g (7.9 mmol) of acetyl imidazole is added with stirring. After 15 min, 30 mL of methanol is added, followed by 15 mL of 1 N NaOH to saponify the ester that is formed along with the amide. The CH₂Cl₂ is removed in vacuo, and the mixture is neutralized with 1 N KH₂PO₄. The product is extracted into ethyl acetate and the organic phase is washed with water, with 1 N NaHCO₃, and with brine. The solution is dried over sodium sulfate, filtered and concentrated to an oil, which is chromatographed over silica gel, eluting with 5%-7% methanol (containing 1% of NH₄OH) in CH₂Cl₂. Product-containing fractions are pooled, concentrated, dissolved in a small volume of ethanol, and acidified with 0.6 N HCl in dry ether. Concentration from this solvent mixture affords a gel-like material. This can be dissolved in ethanol and ethyl acetate, and concentrated to 1.65 g (3.3 mmol, 87%) an off-white solid. This solid is triturated with ethyl acetate to remove a pale yellow mother liquor, leaving hydrochloride 11 as a white solid. ¹H NMR (CDCl₃+CD₃OD drop); δ 7.44 (s, 1 H), 7.37 (m, 2 H), 7.29 (m, 1 H), 6.70 (m, 2 H), 6.62 (m, 1 H), 3.94 (m, 1 H), 3.87 (m, 1 H), 3.0-2.94 (m, 2 H), 2.64 (m, 4 H), 2.36 (m, 1 H), 2.09 (m, 2 H), 1.84 (s, 3 H), 1.79 (m, 2 H), 1.59 (m, 1 H), 1.5-1.3 (m, 3 H), 1.27 (d, J=7 Hz, 6 H); IR (diffuse reflectance); 3343, 3254, 2958, 2937, 2866, 2497, 2442, 2377, 1660, 1628, 1598, 1553, 1460, 1116, cm⁻¹. MS (El) m/z (relative intensity) 458 (M+, 7), 415 (20), 230 (35), 202 (18), 201 (99), 200 (26), 159 (35), 157 (32), 133 (41), 129 (28), 117 (17). HRMS (ESI) calculated for C₂₇H₃₆N₂O₂F₂+H₁ 459.2823, found 459.2837. Anal. Calculated for C₂₇H₃₆F₂N₂O₂.HCl: C, 65.51; H, 7.53; N, 5.66; Cl, 7.16; F, 7.68. Found: C, 65.19; H, 7.70; N, 5.67. Found; Cl, 7.08.

### EXAMPLE 23

### PREPARATION OF N-((1S,2R)-1-(3-(HEXYLOXY)-5-FLUOROBENZYL)-2-HYDROXY-3-{[1-(3-ISOPROPYLPHENYL)CYCLOHEXYL]AMINO}PROPYL)ACETAMIDE HYDROCHLORIDE

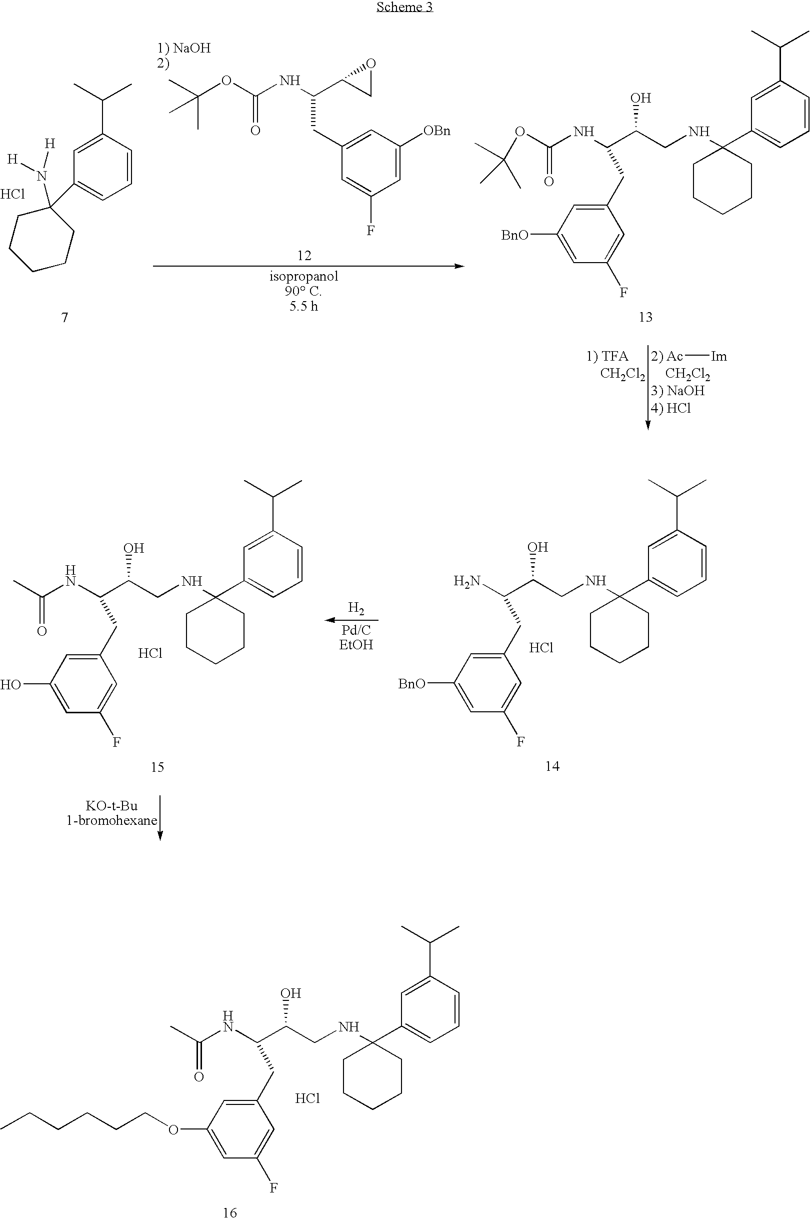

### Step 1. Preparation of tert-butyl (1S,2R)-1-(3-(benzyloxy)-5-fluorobenzyl)-2-hydroxy-3-{[1-(3-isopropylphenyl)cyclohexyl]amino]propylcarbamate (13)

Following essentially the procedure described in Step 1 of EXAMPLE 22, the free base of 1-(3-isopropylphenyl) cyclohexanamine hydrochloride 7 (3.9 mmol) is reacted with tert-butyl (1S)-2-[3-(benzyloxy)-5-fluorophenyl]-1-[(2S)-oxiran-2-yl]ethylcarbamate (12, 0.80 g, 2 mmol) in 20 mL of isopropyl alcohol at reflux overnight. After workup and chromatography over silica gel, eluting with 4% methanol (containing 2% NH₄OH) in CH₂Cl₂, 13 is obtained as a colorless syrup (0.92 g, 1.5 mmol, 74%): MS (Cl) m/z 605.5 (MH+).

### Step 2. Preparation of N-((1S,2R)-1-(3-(benzyloxy)-5-fluorobenzyl)-2-hydroxy-3-{[1-(3-isopropylphenyl)cyclohexyl]amino)propyl)acetamide hydrochloride (14)

Following essentially the procedures of Steps 2 and 3 of EXAMPLE 22, compound 13 (0.92 g, 1.5 mmol) is deprotected with trifluoroacetic acid and reacted with an excess of acetyl imidazole. This is followed by alkaline hydrolysis yielding, after workup and chromatography over silica gel, eluting with 7%-10% methanol (containing 1% NH₄OH) in CH₂Cl₂, and conversion to the HCl salt, 0.75 g (1.3 mmol, 85%) of hydrochloride, 14 as a white solid. ¹H NMR (CDCl₃+CD₃OD drop); δ 7.46-7.25 (m, 9 H), 6.26 (s, 1 H), 6.53-6.47 (m, 2 H), 5.00 (s, 2 H), 4.01 (m, 1 H), 3.88 (m, 1 H), 2.98-2.89 (m, 2 H), 2.68-2.62 (m, 4 H), 2.3 (m, 1H, obscured by water), 2.14 (m, 2 H), 1.88 (s, 3 H), 1.78 (m, 2 H), 1.58 (m, 1 H), 1.5-1.3 (m, 3 H), 1.26 (d, J=7 Hz, 6 H); MS (Cl) m/z 547.5 (MH+).

### Step 3. Preparation of N-((1S,2R)-1-(3-hydroxy-5-fluorobenzyl)-2-hydroxy-3-{[1-(3-isopropylphenyl)cyclohexyl]amino}propyl)acetamide hydrochloride (15)

To a solution of compound 14 (0.70 g, 1.2 mmol) in 70 mL of ethanol in a Parr bottle is added 0.33 g of 10% palladium on carbon. The mixture is placed under 20 psi of hydrogen and shaken for 21 h. The mixture is filtered and the catalyst is washed with ethanol. Concentration in vacuo affords a colorless oil, which is treated with ethereal HCl yielding a quantitative yield of hydrochloride 15 as a white solid. ¹H NMR (CDCl₃+CD₃OD drop); δ 7.44 (s, 1 H), 7.37 (m, 2 H), 7.28 (m, 1 H), 6.59 (s, 1 H), 6.40 (m, 1 H), 6.31 (m, 1 H), 4.0 (m, 1 H), 3.79 (m, 1 H), 2.95 (m, 2 H), 2.63 (m, 4 H), 2.44 (m, 1 H), 2.05 (m, 2 H), 1.90 (s, 3 H), 1.79 (m, 2 H), 1.59 (m, 1 H), 1.5-1.3 (m, 3 H), 1.26 (d, J=7 Hz, 6 H); MS (Cl) m/z 457.4 (MH+).

### Step 4. Preparation of N-((1S,2R)-1-(3-(hexyloxy)-5-fluorobenzyl)-2-hydroxy-3-{[1-(3-isopropylphenyl)cyclohexyl]amino}propyl)acetamide hydrochloride (16)

To 0.40 mmol of N-((1S,2R)-1-(3-hydroxy-5-fluorobenzyl)-2-hydroxy-3-{[1-(3-isopropylphenyl)cyclohexyl]amino)propyl)acetamide hydrochloride 15 in 3 mL of acetone is added 0.29 mL (2.1 mmol) of 1-bromohexane. The mixture is heated to reflux, and 0.6 mL of a 1 M solution of potassium t-butoxide in THF (0.6 mmol) is added. After 1.2 h the mixture is cooled and aqueous 1 N KH₂PO₄ and ethyl acetate are added. The organic phase is washed twice with 1 N NaHCO₃ and once with brine, dried over sodium sulfate, and concentrated. Chromatography over silica gel, eluting with 7%-9% methanol (containing 1% of NH₄OH) in CH₂Cl₂, affords a colorless oil. Treatment with ethereal HCl produces 147 mg (0.25 mmol, 64%) of hydrochloride 16 as a white solid. ¹H NMR (CDCl₃+CD₃OD drop); δ 7.45 (s, 1 H), 7.37 (m, 2 H), 7.27 (m, 1 H), 6.50 (s, 1 H), 6.43 (m, 2 H), 3.98 (m, 1 H), 3.88 (m +t, J=6.5 Hz, 3 H), 2.93 (m, 2 H), 2.63 (m, 4 H), 2.38 (m, 1 H), 2.09 (m, 2 H), 1.89 (s, 3 H), 1.75 (m, 4 H), 1.59 (m, 1 H), 1.43-1.32 (m, 10 H), 1.27 (d, J=7 Hz, 6 H), 0.90 (t, J=7 Hz, 3 H); MS (Cl) m/z 541.5 (MH+).

### EXAMPLE 24

### PREPARATION OF N-((1S,2R)-1-(3-FLUORO-4-HYDROXYBENZYL)-2-HYDROXY-3-{[1-(3-ISOPROPYL PHENYL)CYCLOHEXYL]AMINO}PROPYL)ACETAMIDE HYDROCHLORIDE

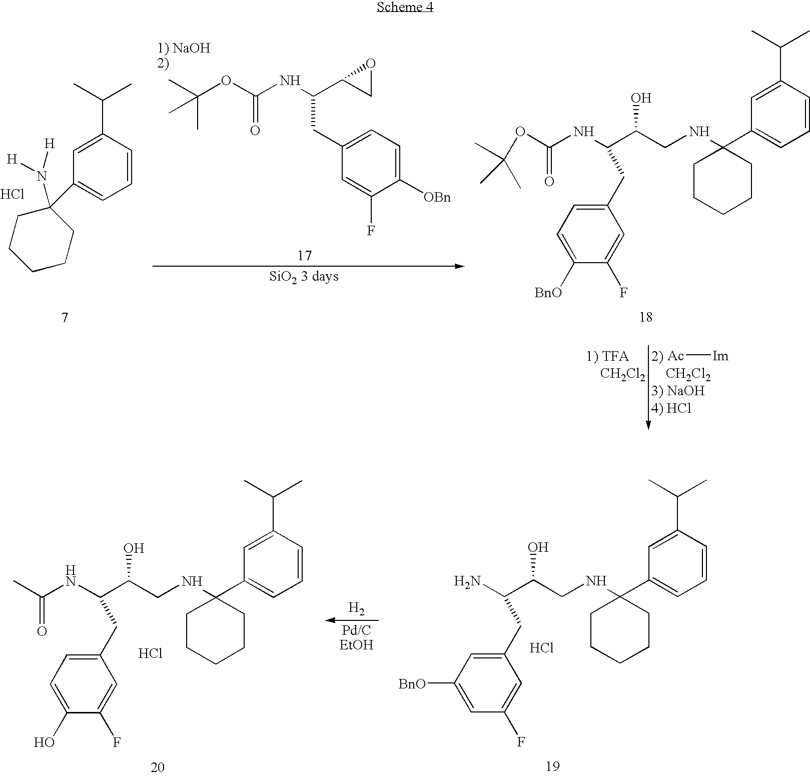

### Step 1. Preparation of tert-butyl (1S,2R)-1-(3-fluoro-4-(benzyloxy)benzyl)-2-hydroxy-3-{[1-(3-isopropylphenyl)cyclohexyl]amino}propylcarbamate (18)

The free base (270 mg, 1.24 mmol) of 1-(3-isopropylphenyl) cyclohexanamine hydrochloride 7 is obtained as a colorless oil by neutralization of the salt with 1 N NaOH, extraction into ethyl acetate, drying over sodium sulfate, and concentration. This is dissolved in 10 mL of CH₂Cl₂, and to it is added tert-butyl (1S)-2-[4-(benzyloxy)-3fluorophenyl]-1-[(2S)-oxiran-2-yl]ethylcarbamate 17 (280 mg, 0.73 mmol) and 1.25 g of silica gel. The solvent is removed in vacuo and the reactants on silica are allowed to stand at ambient temperature for three days. The product mixture is eluted from the silica with 10% methanol in CH₂Cl₂, concentrated, and chromatographed on silica gel, eluting with 4% methanol (containing 2% NH₄OH) in CH₂Cl₂, yielding 18 (238 mg, 0.39 mmol, 54%) as a colorless oil: ¹H NMR (CDCl₃); 5 7.43-7.26 (m, 8 H), 7.12 (m, 1 H), 6.94-6.84 (m, 3 H), 5.09 (s, 2 H), 4.64 (d, J=9 Hz, 1 H), 3.80 (br, 1 H), 3.31 (br, 1 H), 2.92-2.83 (m, 2 H), 2.7 (m, 1 H), 2.37 (m, 2 H), 2.0-1.95 (m, 4 H), 1.67-1.50 (m, 5 H), 1.35 (s+m, 10 H), 1.25 (d, J=7 Hz, 6 H).

### Step 2. Preparation of N-((1S,2R)-1-(3-fluoro-4-(benzyloxy)benzyl)-2-hydroxy-3-{[1-(3-isopropylphenyl)cyclohexyl]amino}propyl)acetamide hydrochloride (19)

Following essentially the procedures of Steps 2 and 3 of EXAMPLE 22, compound 18, (0.238 g, 0.39 mmol) as prepared in step 1, above, is deprotected with trifluoroacetic acid and reacted with an excess of acetyl imidazole. This is followed by alkaline hydrolysis yielding, after workup and chromatography over silica gel, eluting with 7%-10% methanol (containing 1% NH₄OH) in CH₂Cl₂, and conversion to the HCl salt, 0.19 g (0.32 mmol, 75%) hydrochloride, 19 as a white solid. MS (Cl) m/z 547.5 (MH+).

### Step 3. Preparation of N-((1S,2R)-1-(3-fluoro-4-hydroxybenzyl)-2-hydroxy-3-{[1-(3-isopropylphenyl)cyclohexyl]amino}propyl)acetamide hydrochloride (20)

Following essentially the procedure of EXAMPLE 23, Step 3, the product from step 2, compound 19, (0.19 g, 0.32 mmol) is deprotected under 20 psi of H₂ in the presence of 54 mg of 10% palladium on carbon in 3.5 h, affording, after filtration, concentration and treatment with ethereal HCl, 20 (0.16 g, 0.32 mmol, quant.) as a cream-white solid. ¹H NMR (CDCl₃+CD₃OD drop); δ 7.43-7.27 (m, 4 H), 6.86-6.77 (m, 3 H), 3.95 (br, 1 H), 3.8 (br, 1 H), 2.93 (m, 2 H), 2.6 (m, 4 H), 2.4 (m, 1 H), 2.06 (m, 2 H), 1.85 (s, 3 H), 1.8 (m, 2 H), 1.59 (m, 1 H), 1.5-1.3 (m, 3 H), 1.27 (d, J=7 Hz, 6 H); IR (diffuse reflectance); 3251, 3113, 3087, 3061, 3053, 3028, 2956, 2941, 2865, 2810, 1645, 1596, 1520, 1446, 1294 cm⁻¹. MS (Cl) m/z (relative intensity) 457 (MH+,99), 459 (5), 458 (25), 457 (99), 439 (3), 257 (7), 218 (5), 202 (3), 201 (9), 96 (4), 77 (3). HRMS (ESI) calculated for C₂₇H₃₇N₂O₃F+H₁ 457.2866, found 457.2855. Anal. Calc'd for C₂₇H₃₇FN₂O₃.HCl.1.5 H₂O: C, 62.35; H, 7.95; N, 5.39; Found: C, 62.63; H, 7.76; N, 5.47.

### EXAMPLE 25

### PREPARATION OF 8-(3-ISOPROPYLPHENYL)-1,4-DIOXA-SPIRO[4.5]DECANE-8-AMINE ACETATE

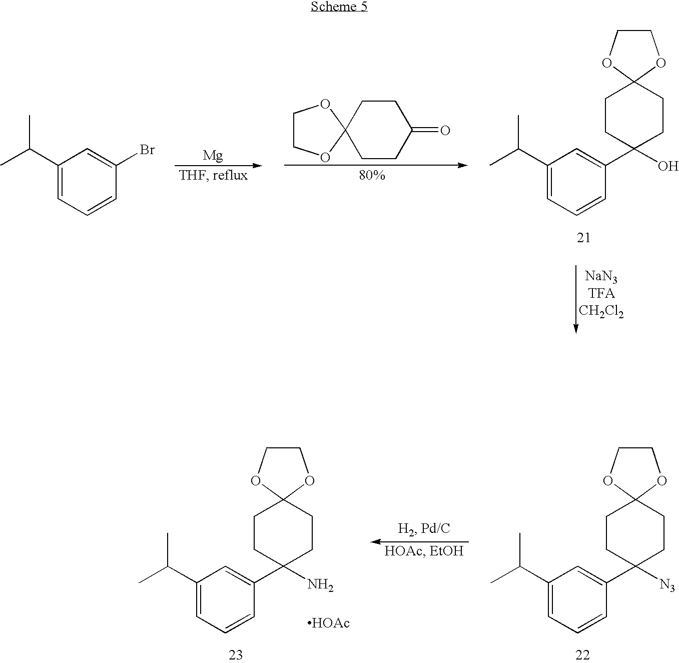

### Step 1. Preparation of 8-(3-isopropylphenyl)-1,4-dioxa-spiro[4.5]decane-8-alcohol (21)

A solution of 3-bromoisopropylbenzene (25 mmol) in 20 mL of dry THF is added dropwise over 20 min to 1.22 g (50 mmol) of magnesium turnings in 10 mL of refluxing THF under nitrogen and the mixture is refluxed for an additional 25 min to form the Grignard reagent. The Grignard solution is cooled and added by cannula to a suspension of CuBr-dimethylsulfide complex (0.52 g, 2.5 mmol) in dry THF at −25° C. The suspension is stirred at −25° C. for 20 min, and then a solution of 1,4 cyclohexanedione, monoethylene ketal (3.9 g, 25 mmol), and 15 mL of THF is added dropwise over 5 min. The mixture is allowed to gradually warm to ambient temperature. Chromatography over silica gel, eluting with 20% to 30% ethyl acetate in heptane, yields alcohol 21 (5.6 g, 20 mmol, 80%), as a colorless oil that crystallizes to a white solid on cooling: ¹H NMR (CDCl₃); δ 7.39 (s, 1 H), 7.33 (m, 1 H), 7.28 (t, J=7.5 Hz, 1 H), 7.13 (d, J=7.5 Hz, 1 H), 4.0 (m, 4 H), 2.91 (hept, J=7 Hz, 1 H), 2.15 (m, 4 H), 1.82 (br d, J=11.5 Hz, 2 H), 1.70 (br d, J=11.5 Hz, 2 H), 1.25 (d, J=7 Hz, 6 H); MS (Cl) m/z 259.2 (M-OH).

### Step 2. Preparation of 8-(3-isopropylphenyl)-1,4-dioxa-spiro[4.5]decane-8-azide (22)

Following essentially the procedure described in EXAMPLE 21, Step 2, 8-(3-isopropylphenyl)-1,4-dioxa-spiro[4.5]decane-8-alcohol 21 (5.5 g, 20 mmol) is reacted with sodium azide (2.9 g, 45 mmol) and trifluoroacetic acid (TFA, 13 mL, 170 mmol) in 120 mL of CH₂Cl₂ at 0° C., allowing the reaction to stir 2 h after dropwise addition of the TFA. The reaction is quenched by dropwise addition of 18 mL of concentrated NH₄OH.

The mixture is taken up in water, ethyl acetate, and heptane, and the organic phase is washed three more times with water and once with brine. The solution is dried (sodium sulfate), filtered, concentrated, and chromatographed over silica gel, eluting with 3% acetone in heptane. Concentration of the product-containing fractions affords 2.2 g (7.3 mmol, 36%) of 22 as a colorless oil: ¹H NMR (CDCl₃); δ 7.33-7.26 (m, 3 H), 7.17 (m, 1 H), 3.98 (m, 4 H), 2.92 (hept, J=7 Hz, 1 H), 2.2-2.12 (m, 2 H), 2.07-1.95 (m, 4 H), 1.72 (m, 2 H), 1.26 (d, J=7 Hz, 6 H).

### Step 3. Preparation of 8-(3-isopropylphenyl)-1,4-dioxa-spiro[4.5]decane-8-amine acetate (23)

Following essentially the procedure described in EXAMPLE 21, Step 3, 2.2 g (7.3 mmol) of 8-(3-isopropylphenyl)-1,4-dioxa-spiro[4.5]decane-8-azide 22 in 200 mL of ethanol is reduced under 16 psi of hydrogen in the presence of 0.7 g of 10% palladium on carbon for 4.5 h. Filtration and removal of solvents with a toluene azeotrope affords a white solid which is triturated with pentane to yield 2.14 g (6.4 mmol, 87%) of 23 as a white solid. ¹H NMR (CDCl₃); δ 7.37-7.33 (m, 2 H), 7.30-7.26 (m, 1 H), 7.13 (d, J=7.5 Hz, 1 H), 5.91 (br, 3 H), 3.96 (m, 4 H), 2.90 (hept., J=7 Hz, 1 H), 2.32 (m, 2 H), 2.03 (s, 3 H), 2.0-1.85 (m, 4 H), 1.63 (m, 2 H), 1.25 (d, J=7 Hz, 6 H); MS (Cl) m/z 259.2 (M-NH₂).

### EXAMPLE 26

### PREPARATION OF N-((1S,2R)-1-(3,5-DIFLUOROBENZYL)-2-HYDROXY-3-{(1-(3-ISOPROPYLPHENYL)CYCLOHEXAN-4-ONE]AMINO}PROPYL) ACETAMIDE

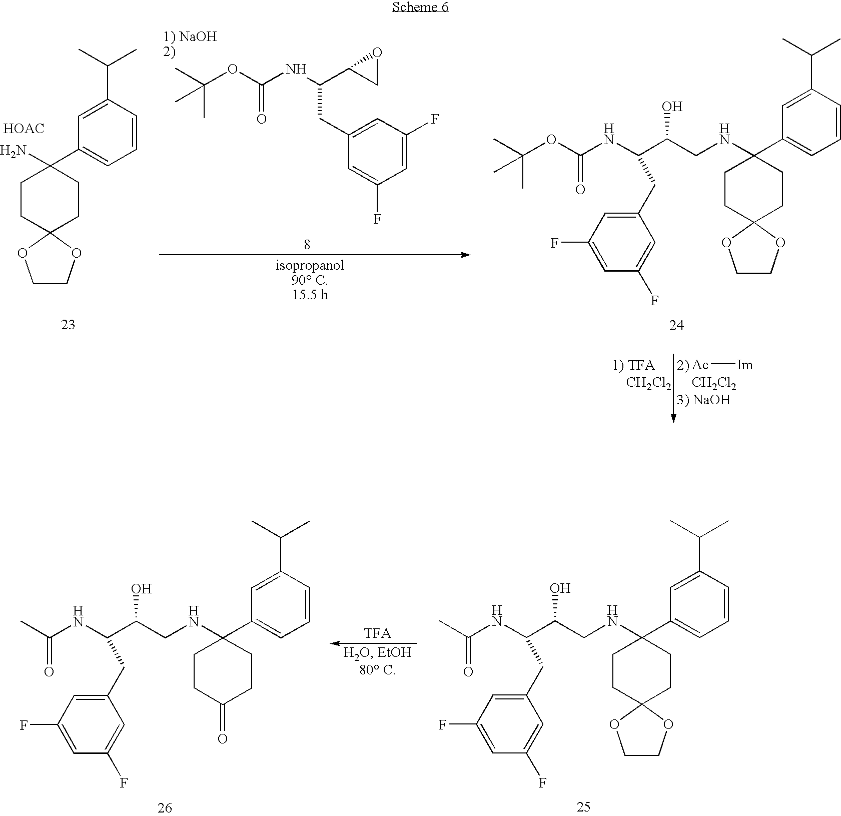

### Step 1. Preparation of tert-butyl (1S,2R)-1-(3,5-difluorobenzyl)-2-hydroxy-3-{8-(3-isopropylphenyl)-1,4-dioxa-spiro[4.5]decane-8-amino}propylcarbamate (24)

Following essentially the procedure of EXAMPLE 22, 8-(3-isopropylphenyl)-1,4-dioxa-spiro[4.5]decane-8-amine acetate 23 (3.2 mmol) is neutralized and reacted with [2-(3,5-Difluoro-phenyl)-1-oxiranyl-ethyl]-carbamic acid tert-butyl ester (8, 0.6 g, 2.0 mmol) in refluxing isopropanol (15 mL) for 15.5 h. The reaction mixture is concentrated and chromatographed over silica gel, eluting with 4% methanol (containing 2% of NH₄OH) in CH₂Cl₂ to separate the crude product from excess 8-(3-isopropylphenyl)-1,4-dioxa-spiro[4.5]decane-8-amine. The crude product is then re-chromatographed over silica gel, eluting with 10% to 20% acetone in CH₂Cl₂ yielding 0.600 g (1.04 mmol, 52%) of 24 as a colorless oil: ¹H NMR (CDCl₃); δ 7.27-7.20 (m, 3 H), 7.09 (d, J=7 Hz, 1 H), 6.69 (m, 2 H), 6.63 (m, 1 H), 4.64 (d, J=9 Hz, 1 H), 3.95 (m, 4 H), 3.72 (m, 1 H), 3.28 (m, 1 H), 2.88 (m, 2 H), 2.69 (dd, J=8.5,14 Hz, 1 H), 2.32 (m, 2 H), 2.15 (m, 2 H), 1.99-1.86 (m, 4 H), 1.63 (m, 2 H), 1.35 (s, 9 H), 1.24 (d, J=7 Hz, 6 H); MS (Cl) m/z 575.4 (MH+)

### Step 2. Preparation of N-((1S,2R)-1-(3,5-difluorobenzyl)-2-hydroxy-3-{8-(3-isopropylphenyl)-1,4-dioxa-spiro[4.5]decane-8-amino}propyl)acetamide (25)

Following essentially the procedures described in EXAMPLE 22, Steps 2 and 3, tert-butyl (1S,2R)-1-(3,5-difluorobenzyl)-2-hydroxy-3-{8-(3-isopropylphenyl)-1,4-dioxa-spiro[4.5]decane-8-amino}propylcarbamate 24 (0.600 g, 1.04 mmol) is deprotected, acetylated, and saponified yielding, after chromatography on silica gel and eluting with 32.5% acetone and 2.5% methanol in CH₂Cl₂, acetamide 25 (335 mg, 0.65 mmol, 62%) as a white solid by concentration from ethyl ether: ¹H NMR (CDCl₃); δ 7.31-7.26 (m, 3 H), 7.15 (m, 1 H), 6.69-6.61 (m, 3 H), 5.9 (br, 1 H), 4.13 (m, 1 H), 3.95 (m, 4 H), 3.48 (m, 1 H), 2.92-2.83 (m, 2 H), 2.73 (dd, J=8.5, 14 Hz, 1 H), 2.45-2.25 (m, 4 H), 2.10 (m, 2 H), 1.88 (s+m, 5 H), 1.62 (m, 2 H), 1.25 (d, J=7 Hz, 6 H); MS (Cl) m/z 517.4 (MH+).

### Step 3. Preparation of N-((1S,2R)-1-(3,5-difluorobenzyl)-2-hydroxy-3-{[1-(3-isopropylphenyl)cyclohexan-4-one]amino}propyl)acetamide (26)

To N-((1S,2R)-1-(3,5-difluorobenzyl)-2-hydroxy-3-{8-(3-isopropylphenyl)-1,4-dioxa-spiro[4.5]decane-8-amino}propyl) acetamide 25 (255 mg, 0.49 mmol) in 5 mL of ethanol and 5 mL of water is added 6 mL of trifluoroacetic acid, and the mixture is refluxed for 2 h under nitrogen. It is concentrated and taken up in aqueous 10% Na₂CO₃ and ethyl acetate. The organic phase is washed twice more with 10% Na₂CO₃ and then with brine. It is dried over sodium sulfate, and concentrated to a colorless oil. Evaporation in vacuo from ethyl ether affords 26 (140 mg, 0.30 mmol, 60%) as a white solid. ¹H NMR (CDCl₃); δ 7.35-7.18 (m, 4 H), 6.71-6.64 (m, 3 H), 5.65 (br, 1 H), 4.12 (m, 1 H), 3.43 (m, 1 H), 2.95-2.90 (m, 2 H), 2.75 (dd, J=8.5,14 Hz, 1 H), 2.64 (m, 2 H), 2.4-2.25 (m, 8 H), 1.87 (s, 3 H), 1.25 (d, J=7 Hz, 6 H); MS (Cl) m/z 473.4 (MH+). The LC-MS spectrum in methanol solvent shows a small signal at 505.4 (MH+CH₃OH) due to hemiketal formation. IR (diffuse reflectance); 3311, 2958, 1710, 1646, 1628, 1595, 1550, 1544, 1460, 1372, 1315, 1116, 983, 846, 707 cm⁻¹.

MS (El) m/z (relative intensity) 472 (M+, 6), 472 (6), 417 (5), 416 (33), 415 (99), 398 (8), 397 (30), 327 (11), 244 (9), 215 (13), 214 (6). HRMS (ESI) calculated for C₂₇H₃₄N₂O₃F₂+H₁ 473.2615, found 473.2627. Anal. Calc'd for C₂₇H₃₄F₂N₂O₃+0.5 H₂O: C, 67.34; H, 7.33; N, 5.82; Found (av): C, 67.89; H, 7.32; N, 5.86.

### EXAMPLE 27

### PREPARATION OF (1S,2R)-1-(3,5-DIFLUOROBENZYL)-2-HYDROXY-3-{[1-(3-ISOPROPYLPHENYL)CYCLOHEXYL]AMINO}PROPYLFORMAMIDE HYDROCHLORIDE

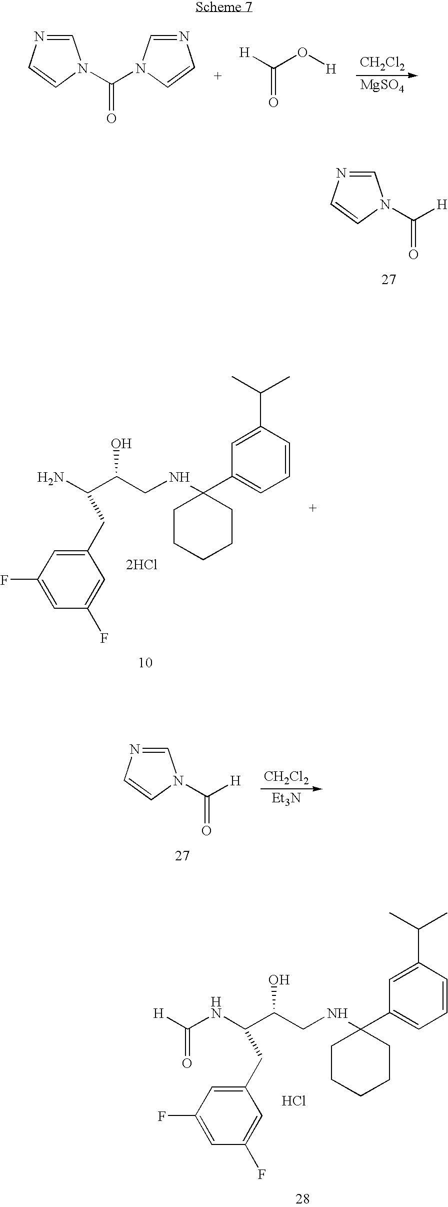

### Step 1. Preparation of formyl imidazole (27)

To a solution of formic acid (0.76 mL, 20 mmol, 96%) in CH₂Cl₂ stirring under nitrogen is added, portion-wise over 10 min, 3.6 g (22 mmol) of carbonyldiimidazole, and the mixture is allowed to stir overnight. Anhydrous magnisium sulfate is added, and after several hours the mixture is filtered and concentrated in vacuo (note: formyl imidazole is volatile and this operation should be carefully monitored for maximum recovery) yielding 0.7 g of iridescent crystals. The NMR spectrum showed the presence of formyl imidazole 27: ¹H NMR (CDCl₃); δ 9.15 (s, 1 H), 8.14 (s, 1 H), 7.53 (s, 1 H), 7.20 (s, 1 H). The crystals also contain imidazole (6 7.71 (s,1 H), 7.13 (s, 2H)) and the relative peak intensity and relative molecular weights are used to determine the weight % of formyl imidazole in the product.

### Step 2. Preparation of (1S,2R)-1-(3,5-difluorobenzyl)-2-hydroxy-3-{[1-(3-isopropylphenyl)cyclohexyl]amino}propylformamide hydrochloride (28)

To a solution of (2R,3S)-3-amino-4-(3,5-difluorophenyl)-1-{[1-(3-isopropylphenyl) cyclohexyl]amino}butan-2-ol dihydrochloride 10 (209 mg, 0.43 mmol) in 4 mL of CH₂Cl₂ under nitrogen is added 125 μL (0.9 mmol) of triethylamine. To this mixture is added 75 mg of the solid from Step 1, which is determined by NMR to contain 63% by weight of formyl imidazole (47 mg, 0.49 mmol) and the solution is stirred for 20 min. Methanol (5 mL) is added, followed by 2 mL of 1 N NaOH. The mixture is concentrated in vacuo and diluted with 1 N KH₂PO₄ and ethyl acetate. The organic phase is washed with 1 N NaHCO₃ and brine, and dried over sodium sulfate. Concentration and chromatography over silica gel, eluting with 5% to 7.5% of methanol (containing 1% of NH₄0H) in CH₂Cl₂ affords a colorless oil. Ether and ethereal HCl are added, and the gel-like precipitate is concentrated in vacuo from ethanol and then ethyl acetate yielding 176 mg (0.37 mmol, 85%) of hydrochloride 28 as a white solid. ¹H NMR (CDCl₃+CD₃OD drop); 8 7.86 (s, 1 H), 7.39-7.28 (m, 4 H), 6.67 (m, 2 H), 6.60 (m, 1 H), 3.96 (m, 1 H), 3.79 (m, 1 H), 3.08 (dd, 1 H), 2.93 (m, 1 H), 2.7-2.5 (m, 4 H), 2.37 (dd, 1 H), 2.05 (m, 2 H), 1.78 (m, 2 H), 1.6 (m, 1 H), 1.45-1.3 (m, 3 H), 1.25 (dd, J=1, 7 Hz, 6 H); MS (Cl) m/z 445.3 (MH+).

### EXAMPLE 28

### PREPARATION OF N-((1S,2R)-1-(3,5-DIFLUOROBENZYL)-2-HYDROXY-3-{[1-(3-ISOPROPYLPHENYL)CYCLOHEXYL]AMINO}PROPYL)-2-FLUOROACETAMIDE HYDROCHLORIDE

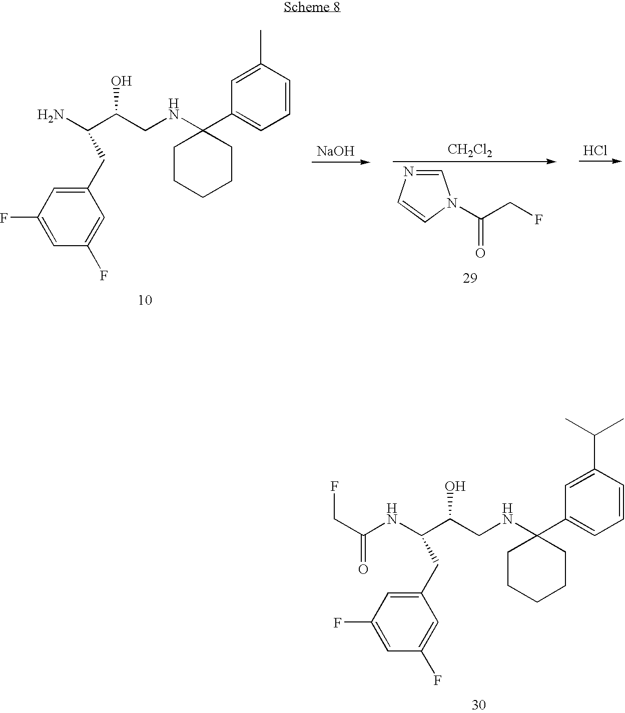

### Step 1. Preparation of fluoroacetyl imidazole (29). Fluoroacetyl imidazole 29 is obtained according to EXAMPLE 7.

### Step 2. Preparation of N-((1S,2R)-1-(3,5-difluorobenzyl)-2-hydroxy-3-{[1-(3-isopropylphenyl)cyclohexyl]amino}propyl)-2-fluoroacetamide hydrochloride (30)

To (2R,3S)-3-amino-4-(3,5-difluorophenyl)-1-{[1-(3-isopropylphenyl)cyclohexyl]amino}butan-2-ol dihydrochloride 10 (0.64 mmol) is added 1 N NaOH and ethyl acetate. The organic phase is washed with more 1 N NaOH, brine, and then dried over sodium sulfate and concentrated to 265 mg of a colorless oil. This free base is dissolved in 3 mL of CH₂Cl₂ under nitrogen and 3.2 mL (0.64 mmol) of a 0.2 M solution of fluoroacetyl imidazole 29 in CH₂Cl₂ is added. The mixture is stirred for 5 min, and then aqueous 1 N KH₂PO₄ and ethyl acetate are added. The organic phase is washed with 1 N KH₂PO₄, 1 N NaHCO₃, and brine, dried over sodium sulfate, and concentrated. Chromatography over silica gel, eluting with 5% methanol (containing 2% of NH₄OH) in CH₂Cl₂ affords a colorless oil. Ether and ethereal HCl are added, and the solvents are removed in vacuo to yield 256 mg (0.50 mmol, 78%) of hydrochloride 30 as a white solid. ¹H NMR (CDCl₃); δ 9.85 (m, 1 H), 8.0 (m, 1 H), 7.51 (s, 1 H), 7.37 (m, 2 H), 7.27 (m, 1 H), 6.80 (d, J=7 Hz, 1 H), 6.68 (m, 2 H), 6.63 (m, 1 H), 4.63 (d, J=47 Hz, 2 H), 4.16 (m, 1 H), 4.10 (m, 1 H), 2.98-2.93 (m, 2 H), 2.77-2.64 (m, 4 H), 2.35-2.2 (m, 3 H), 1.80 (m, 2 H), 1.59 (m, 1 H), 1.44-1.25 (m, 3 H), 1.28 (d, J=7 Hz, 6 H); MS (Cl) m/z 477.4 (MH+).

### EXAMPLE 29

### PREPARATION OF N-((1S,2R)-1-(3,5-DIFLUOROBENZYL)-2-HYDROXY-3-{[1-(3-ISOPROPYLPHENYL) CYCLOHEXYL]AMINO}PROPYL)ETHANETHIOAMIDE HYDROCHLORIDE

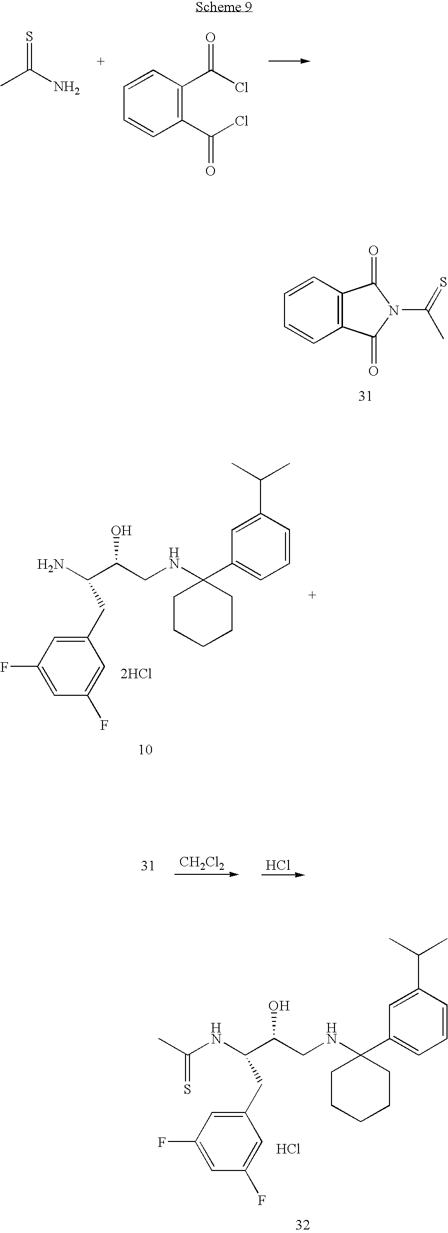

### Step 1. Preparation of thioacetyl-N-phthalimide (32)

Thioacetamide (1.9 g, 25 mmol) is suspended in 40 mL of CH₂Cl₂ and cooled in an ice bath under nitrogen. Phthaloyidichloride (3.6 mL, 25 mmol) is added slowly over 10 min via syringe while the mixture is stirred. The mixture becomes a clear orange solution transiently, eventually depositing a precipitate. After stirring for 40 h, the mixture is concentrated in vacuo. The oily coral solid is triturated with hexanes. Within minutes the hexane mother liquor drops a precipitate, which is filtered off yielding 0.2 g of a light coral solid. ¹H NMR (CDCl₃); 8 7.99 (m, 2 H), 7.86 (m, 2 H), 3.08 (s, 3 H). The residual solids remaining after trituration with hexanes are further triturated with ether and then with CH₂Cl₂. The combined mother liquors are concentrated to about 3 g of a red oily solid, which is chromatographed over silica gel, eluting with 10% to 20% ethyl acetate in heptane. The red fractions contained a product (concentrated to a coral solid, 0.77 g) with the same TLC retention (R_{f}=0.32, 20% ethyl acetate in heptane) as the coral solid which had precipitated from hexanes. The total recovery is 0.97 g, 4.7 mmol, 19%.

### Step 2. Preparation of N-((1S,2R)-1-(3,5-difluorobenzyl)-2-hydroxy-3-{[1-(3-isopropylphenyl)cyclohexyl]amino}propyl)ethanethioamide hydrochloride (32)

To 164 mg (0.39 mmol) of the free base prepared from (2R,3S)-3-amino-4-(3,5-difluorophenyl)-1-{[1-(3-isopropylphenyl)cyclohexyl]amino}butan-2-ol dihydrochloride 10 and dissolved in 3 mL of CH₂Cl₂ under nitrogen, cooled in an ice bath, is added solid thioacetyl-N-phthalimide 31 (80 mg, 0.39 mmol). The mixture is stirred for 20 min, and then 3 mL of methanol and 3 mL of 1 N NaOH are added. The mixture is taken up in ethyl acetate and washed twice with 1 N NaOH, once with water, and once with brine. It is dried over sodium sulfate, concentrated, and chromatographed over silica gel, eluting with 4% methanol (containing 2% NH₄OH) in CH₂Cl₂. Product-containing fractions are concentrated to a colorless oil, which is dissolved in ether and treated with ethereal HCl. Concentration affords 97 mg (0.19 mmol, 49%) of hydrochloride 32 as a white solid. ¹H NMR (CDCl₃+CD₃OD drop); 6 7.42-7.37 (m, 2 H), 7.29 (m, 2 H), 6.73 (m, 2 H), 6.62 (m, 2 H), 4.67 (m, 1 H), 4.10 (m, 1 H), 3.11 (dd, J=5, 14 Hz, 1 H), 2.96 (hept, J=7 Hz, 1 H), 2.83 (m, 1 H), 2.65-2.4 (m, 4 H, obscured by solvent), 2.38 (s, 3 H), 2.07 (m, 2 H), 1.78 (m, 2 H), 1.59 (m, 1 H), 1.44-1.35 (m, 3 H), 1.28 (d, J=7 Hz, 6 H); MS (Cl) m/z 475.3 (MH+).

### EXAMPLE 30

### PREPARATION OF N-((1S,2R)-2-HYDROXY-1-(4-HYDROXYBENZYL)-3-{[1-(3-ISOPROPYLPHENYL)CYCLOHEXYL]AMINO}PROPYL)ACETAMIDE HYDROCHLORIDE

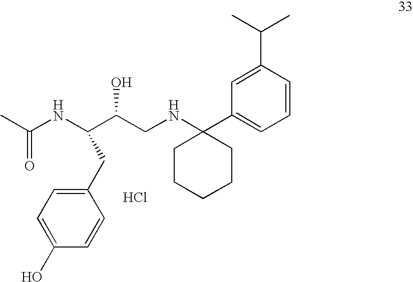

Using methods analogous to those previously described, tert-butyl (1 S)-2-(4-hydroxyphenyl)-1-[(2S)-oxiran-2-yl]ethylcarbamate (0.78 mmol) is converted to the N-((1S ,2R)-2-hydroxy-1-(4-hydroxybenzyl)-3-{[1-(3-isopropylphenyl) cyclohexyl]amino}propyl)acetamide hydrochloride 33 (70 mg, 0.15 mmol, 19%, 3 steps), which is obtained as a white solid. ¹H NMR (CDCl₃+CD₃OD drop); δ 7.49 (s, 1 H), 7.39 (d, J=4.6 Hz, 2 H), 7.28 (m, 1 H), 6.91 (d, J=8 Hz, 2 H), 6.69 (d, J=8Hz, 2 H), 3.97 (m, 1 H), 3.90 (m, 1 H), 2.96 (hept, J=7 Hz, 1 H), 2.83 (dd, 1 H), 2.62 (m, 4 H), 2.45 (m, 1 H), 2.13 (m, 2 H), 1.89 (s, 3 H), 1.78 (m, 2 H), 1.58 (m, 1 H), 1.45-1.3 (m, 3 H), 1.27 (d, J=7 Hz, 6 H); MS (Cl) m/z 439.3 (MH+).

### EXAMPLE 31

### PREPARATION OF N-((1S,2R)-1-[3-(ALLYLOXY)-5-FLUOROBENZYL]-2-HYDROXY-3-([1-(3-ISOPROPYLPHENYL) CYCLOHEXYL]AMINO}PROPYL)ACETAMIDE HYDROCHLORIDE

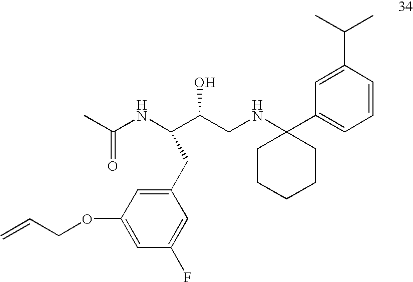

Using methods analogous to those previously described, tert-butyl (1S)-2-[3-(allyloxy)-5-fluorophenyl]-1-[(2S)-oxiran-2-yl]ethylcarbamate (0.61 mmol) is converted to the title compound 34 (0.31 mmol, 51%, 3 steps), which is obtained as a white solid. ¹H NMR (CDCl₃+CD₃OD drop); δ 7.42-7.27 (m, 4 H), 6.54 (m, 1 H), 6.48 (m, 1 H), 6.45 (m, 1 H), 6.05-5.98 (m, 1 H), 5.39 (m, 1 H), 5.28 (m, 1 H), 4.48 (m, 2 H), 3.95 (m, 1 H), 3.77 (m, 1 H), 2.96 (m, 2 H), 2.60 (m, 4 H), 2.4 (m, obscured, 1 H), 2.1 (m, 2 H), 1.81 (s+m, 5 H), 1.6 (m, 1 H), 1.45-1.3 (m, 3 H), 1.27 (d, J=7 Hz, 6 H); MS (Cl) m/z 497.4 (MH+).

### EXAMPLE 32

### PREPARATION OF N-[(1S,2R)-2-HYDROXY-3-([1-(3-ISOPROPYLPHENYL)CYCLOHEXYL]AMINO}-1-(THIEN-2-YLMETHYL)PROPYL]ACETAMIDE HYDROCHLORIDE

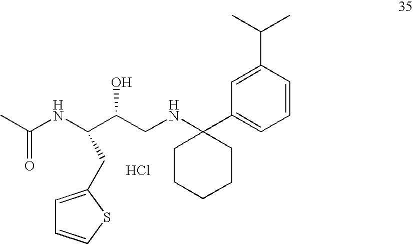

Using methods analogous to those previously described, tert-butyl (1 S)-1-[(2S)-oxiran-2-yl]-2-thien-2-ylethylcarbamate (0.92 mmol) is converted to the title compound 35 (0.51 mmol, 55%, 3 steps), which is obtained as a white solid. ¹H NMR (CDCl₃); δ 9.8 (br, 1 H), 8.03 (br, 1 H), 7.47 (s, 1 H), 7.37 (m, 2 H), 7.26 (m, 1 H), 7.21 (m, 1 H), 7.0 (br, 1 H), 6.95 (m, 1 H), 6.90 (d, J=5 Hz, 1 H), 4.15 (m, 1 H), 3.96 (m, 1 H), 3.9 (v br, 1 H), 2.96 (hept, J=7 Hz, 1 H), 2.86 (m, 2 H), 2.7-2.55 (m, 3 H), 2.24 (m, 3 H), 2.00 (s, 3 H), 1.8-1.7 (m, 2 H), 1.59 (m, 1 H), 1.45-1.3 (m, 3 H), 1.28 (dd, J=1.7, 7 Hz, 6 H); MS (Cl) m/z 429.3 (MH+).

### EXAMPLE 33

### PREPARATION OF N-((1S,2R)-2-HYDROXY-1-(3-HYDROXYBENZYL)-3-{[1-(3-ISOPROPYLPHENYL)CYCLOHEXYL]AMINO}PROPYL)ACETAMIDE HYDROCHLORIDE

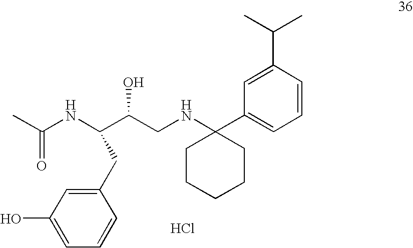

Using methods analogous to those previously described, tert-butyl (1S)-2-[3-(benzyloxy)phenyl]-1-[(2S)-oxiran-2-yl]ethylcarbamate (1.0 mmol) is converted to the N-((1S,2R)-2-hydroxy-1-(3-hydroxybenzyl)-3-{[1-(3-isopropylphenyl) cyclohexyl]amino}propyl)acetamide hydrochloride 36 (0.28 mmol, 28%, 4 steps), obtained as a colorless glass-like solid which can be pulverized into a beige powder: ¹H NMR (CDCl₃+CD₃OD drop); δ 7.43 (s, 1 H), 7.37 (m, 2 H), 7.28 (m, 1 H), 7.08 (t, J=7.7 Hz, 1 H), 6.78 (s, 1 H), 6.69 (d, J=8 Hz, 1 H), 6.57 (d, J=7.5 Hz, 1 H), 4.03 (m, 1 H), 3.75 (m, 1 H), 2.97 (m, 2 H), 2.65 (m, 4 H), 2.43 (m, 1 H), 2.12-2 (m, 2 H), 1.85 (s, 3 H), 1.78 (m, 2 H), 1.59 (m, 1 H), 1.45-1.3 (m, 3 H), 1.27 (d, J=7 Hz, 6 H); MS (Cl) m/z 439.3 (MH+).

### EXAMPLE 34

### PREPARATION OF N-((1S,2R)-1-(3-FLUOROBENZYL)-2-HYDROXY-3-{[1-(3-ISOPROPYLPHENYL) CYCLOHEXYL]AMINO}PROPYL)ACETAMIDE HYDROCHLORIDE

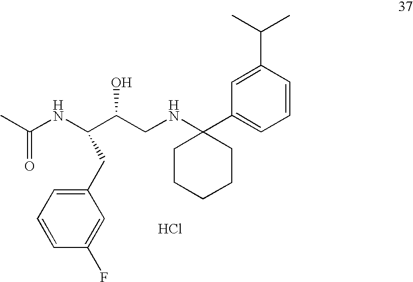

Using methods analogous to those previously described, tert-butyl (1S)-2-(3-fluorophenyl)-1-[(2S)-oxiran-2-yl]ethylcarbamate (0.82 mmol) is converted to the N-((1S,2R)-1-(3-fluorobenzyl)-2-hydroxy-3-{[1-(3-isopropylphenyl)cyclohexyl]amino}propyl)acetamide hydrochloride 37 (0.37 mmol, 45%, 3 steps), obtained as a white solid. ¹H NMR (CDCl₃+CD₃OD drop); δ 7.45 (s, 1 H), 7.4-7.35 (m, 2 H), 7.28 (m, 2 H), 7.20 (m, 1 H), 6.93 (m, 1 H), 6.88 (m, 2 H), 4.00 (m, 1 H), 3.87 (m, 1 H), 2.96 (m, 2 H), 2.7-2.6 (m, 4 H), 2.39 (m, 1 H), 2.11 (m, 2 H), 1.88 (s, 3 H), 1.79 (m, 2 H), 1.59 (m, 1 H), 1.45-1.3 (m, 3 H), 1.27 (d, J=7 Hz, 6 H); MS (Cl) m/z 441.5 (MH+).

### EXAMPLE 35

### PREPARATION OF N-((1S,2R)-1-(3-(HEPTYLOXY)-5-FLUOROBENZYL)-2-HYDROXY-3-{[1-(3-ISOPROPYLPHENYL)CYCLOHEXYL]AMINO}PROPYL)ACETAMIDE HYDROCHLORIDE

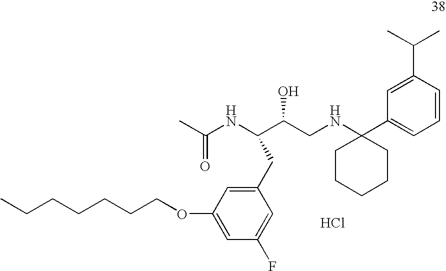

Using methods analogous to those previously described, N-((1S,2R)-1-(3-hydroxy-5-fluorobenzyl)-2-hydroxy-3-{[1-(3-isopropylphenyl)cyclohexyl]amino}propyl)acetamide hydrochloride 15 (0.4 mmol) is reacted with 1-bromoheptane yielding the N-((1S,2R)-1-(3-(heptyloxy)-5-fluorobenzyl)-2-hydroxy-3-{[1-(3-isopropylphenyl)cyclohexyl]amino}propyl)acetamide hydrochloride 38 (0.14 mmol, 34%) as a glass which can be pulverized to an off-white solid. ¹H NMR (CDCl₃+CD₃OD drop); δ 7.49 (s, 1 H), 7.37 (m, 2 H), 7.27 (m, 1 H), 6.51 (s, 1 H), 6.45 (s, 1 H), 6.43 (s, 1 H), 4.05 (m, 1 H), 3.98 (m, 1 H), 3.88 (t, J=6.5 Hz, 2 H), 2.96 (hept, J=7 Hz, 1 H), 2.84 (m, 1 H), 2.6 (3H obscured by solvent), 2.36 (m, 1 H), 2.16 (m, 2 H), 2.01 (s, 3 H), 1.85-1.75 (m, 4 H), 1.58 (m, 1 H), 1.5-1.26 (m, 18 H), 0.89 (t, J=6.6 Hz, 3 H); MS (Cl) m/z 555.5 (MH+).

### EXAMPLE 36

### PREPARATION OF N-((1S,2R)-1-(3-(2-(2-METHOXYETHOXY)ETHOXY)-5-FLUOROBENZYL)-2-HYDROXY-3-{[1-(3-ISOPROPYLPHENYL)CYCLOHEXYL]AMINO}PROPYL)ACETAMIDE HYDROCHLORIDE

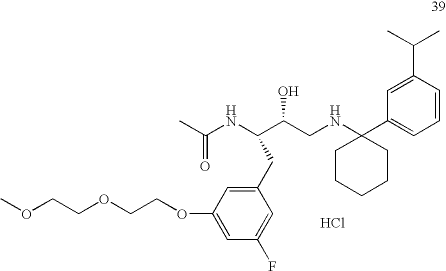

Using methods analogous to those previously described, compound 15 (0.4 mmol) is reacted with 1-bromo-2-(2-methoxyethoxy)ethane yielding the N-((1S,2R)-1-(3-(2-(2-methoxyethoxy) ethoxy)-5-fluorobenzyl)-2-hydroxy-3-{[1-(3-isopropylphenyl)cyclohexyl]amino}propyl) acetamide hydrochloride 39 (0.21 mmol, 52%) as a hygroscopic white solid. ¹H NMR (CDCl₃); δ 9.4 (br, 1 H), 8.5 (br, 1 H), 8.32 (br, 1 H), 7.54 (s, 1 H), 7.38 (m, 2 H), 7.26 (m, 1 H), 6.56 (s, 1 H), 6.47 (m, 2 H), 4.34 (v br, water H), 4.1 (m, 4 H), 3.83 (m, 2 H), 3.70 (m, 2 H), 3.58 (m, 2 H), 3.38 (s, 3 H), 2.96 (hept, J=7 Hz, 1 H), 2.8-2.6 (m, 5 H), 2.4-2.2 (m, 3 H), 2.15 (s, 3 H), 1.80 (m, 2 H), 1.6 (m, 1 H), 1.5-1.3 (m, 3 H), 1.27 (d, J=7 Hz, 6 H); MS (Cl) m/z 559.5 (MH+).

### EXAMPLE 37

### PREPARATION OF N-((1S,2R)-1-[3-(ALLYLOXY)-5-FLUOROBENZYL]-3-{[(4R)-6-ETHYL-2,2-DIOXIDO-3,4-DIHYDRO-1H-ISOTHIOCHROMEN-4-YL]AMINO)-2-HYDROXYPROPYL)ACETAMIDE

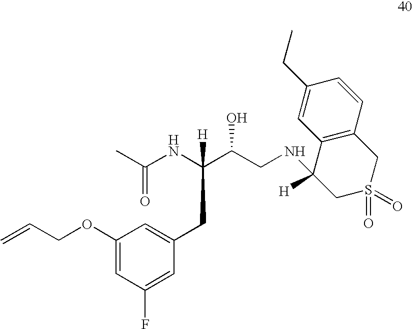

Using methods analogous to those previously described, tert-butyl (1S)-2-[3-(allyloxy)-5-fluorophenyl]-1-[(2S)-oxiran-2-yl]ethylcarbamate (0.37 mmol) and (4R)-6-ethyl-3,4-dihydro-1H-isothiochromen-4-amine 2,2-dioxide (0.78 mmol) are reacted together, and the product is further converted, using methods analogous to those previously described, (except that the HCl salt is not formed) to the N-((1S,2R)-1-[3-(allyloxy)-5-fluorobenzyl]-3-{[(4R)-6-ethyl-2,2-dioxido-3,4-dihydro-1H-isothiochromen-4-yl]amino}-2-hydroxypropyl)acetamide 40 (0.16 mmol, 43%), which is obtained as a white solid. ¹H NMR (CDCl₃); δ 7.22-7.19 (m, 2 H), 7.13 (m, 1 H), 6.57 (m, 1 H), 6.51 (m, 2 H), 6.06-5.99 (m, 1 H), 5.75 (br, 1 H), 5.41 (d, J=17 Hz, 1 H), 5.30 (d, J=12 Hz, 1 H), 4.67 (d, J=15 Hz, 1 H), 4.50 (m, 2 H), 4.26 (m, 1 H), 4.17 (d, J=15 Hz, 1 H), 4.1 (m, 1 H), 3.66 (m, 2 H), 3.48 (m, 1 H), 3.36 (dd, 1 H), 2.90 (m, 2 H), 2.78 (m, 2 H), 2.67 (q, J=7.6 Hz, 2 H), 1.91 (s, 3 H), 1.25 (t, J=7.6 Hz, 3 H); MS (Cl) m/z 505.4 (MH+).

### EXAMPLE 38

### PREPARATION OF 1-TERT-BUTYL-3-IODO-BENZENE FROM 3-(TERT-BUTYL)ANILINE

3-(tert-Butyl)aniline (Oakwood, 6.0 g, 40.21 mmol) was slowly added to a cold solution of 12 N HCl (24.5 mL) while stirring over an ice/acetone bath in a three-neck round-bottom flask equipped with a thermometer. A 2.9M solution of sodium nitrite (16 mL) was added via addition funnel to the reaction flask at a rate so as maintain the temperature below 2° C. The solution was stirred for 30 min. prior to being added to a reaction flask containing a 4.2M solution of potassium iodide (100 mL). The reaction mixture was allowed to stir overnight while warming to room temperature. The mixture was then extracted with a hexane/ether solution (1:1) followed by washing with H₂O (2×), 0.2 N citric acid (2×) and saturated NaCl. The organic phase was separated, dried (sodium sulfate) and concentrated under reduced pressure. The residue was purified by flash chromatography (100% Hexane) yielding the desired iodo intermediate (8.33 g, 80%); ¹H NMR (CDCl₃, 300 MHz) δ 1.34 (s, 9H), 7.07 (t, J=8.0 Hz, 1 H), 7.39 (d, J=8.0 Hz, 1H), 7.55 (d, J=8.0 Hz, 1H), 7.77 (t, J=2.0 Hz, 1H).

### EXAMPLE 39

### PREPARATION OF 1-(3-TERT-BUTYL-PHENYL)-CYCLOHEXANOL FROM 1-TERT-BUTYL-3-IODO-BENZENE

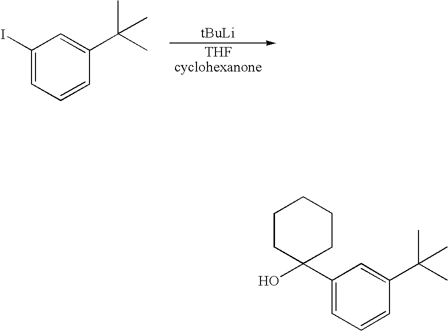

1-tert-Butyl-3-iodo-benzene (8.19 g, 31.49 mmol) in anhydrous THF (35 mL) was cooled to −78° C. A solution of 1.7M tert-butyl lithium was added and the reaction mixture was allowed to stir while under N₂ (g) inlet for 2 h. A solution of cyclohexanone in anhydrous THF (5 mL) was added and the reaction mixture was stirred for 1 h before transferring to a 0° C. bath for 1 h and warming to room temperature for 1 h. The reaction was quenched with H₂O and extracted with ether. The organic layer was separated, dried (sodium sulfate) and concentrated under reduce pressure. The residue was purified by flash chromatography (100% CHCl₃) yielding the desired alcohol (4.73 g, 65%): mass spec (Cl) 215.2 (M-OH).

### EXAMPLE 40

### PREPARATION OF 1-(1-AZIDO-CYCLOHEXYL)3-TERT-BUTYL-BENZENE FROM 1-(3-TERT-BUTYL-PHENYL)-CYCLO HEXANOL

1-(3-tert-Butyl-phenyl)-cyclohexanol (3.33 g, 14.34 mmol) in dry chloroform (75 mL) was cooled to 0° C. under N₂ (g) inlet. Sodium azide (2.89 g, 44.45 mmol) was added followed by dropwise addition of trifluoroacetic acid (5.5 mL, 71.39 mmol). The reaction mixture was allowed to stir at room temperature overnight and then partitioned between H₂O and ether. The aqueous layer was removed and the mixture was washed with H₂O followed by 1.0N NH₄OH. The organic layer was separated, dried (sodium sulfate), and concentrated under reduced pressure. The residue was purified by flash chromatography (100% hexane) yielding the desired azide (0.50 g, 14%): mass spec (Cl) 215.2 (M-N₃).

### EXAMPLE 41

### PREPARATION OF 1-(3-TERT-BUTYL-PHENYL)-CYCLOHEXYLAMINE FROM 1-(1-AZIDO-CYCLOHEXYL)3-TERT-BUTYL-BENZENE

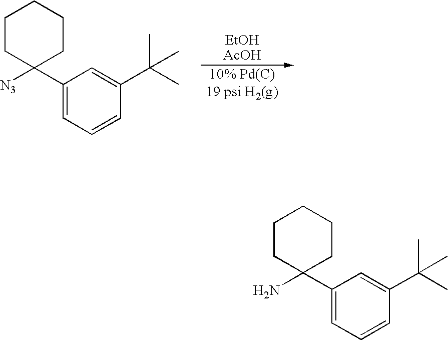

To a solution of 1-(1-Azido-cyclohexyl)-3-tert-butylbenzene dissolved in ethanol (5 mL) was added acetic acid (0.5 mL) and 10% palladium on carbon (0.10 g, 0.94 mmol). The reaction mixture was placed on the hydrogenator at 19 psi for 3.5 h and then filtered through Celite and rinsed with ethanol. The filtrate was collected and concentrated under reduced pressure. This was then partitioned between EtOAc and 1 N NaOH. The aqueous layer was removed and the mixture was washed with H₂O. The organic layer was separated, dried (sodium sulfate), and concentrated under reduced pressure. The crude product was used without further purification: mass spec (Cl) 215.2 (M-NH₂).

### EXAMPLE 42

### PREPARATION OF (1S,2R)-N-[3-[1-(3-TERT-BUTYL-PHENYL)CYCLOHEXYLAMINO]-1-(3,5-DIFLUOROBENZYL)-2-HYDROXY-PROPYL]

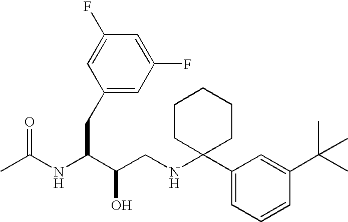

The product from EXAMPLE 41 was converted into the above titled product using methods described in EXAMPLE 22. Mass spec: (Cl) 473.2 (M+H).

### EXAMPLE 43

### PREPARATION OF 1-(3-ETHYNYLPHENYL)CYCLOHEXYLAMINE FROM 1-(3-BROMO-PHENYL)-CYCLOHEXYLAMINE

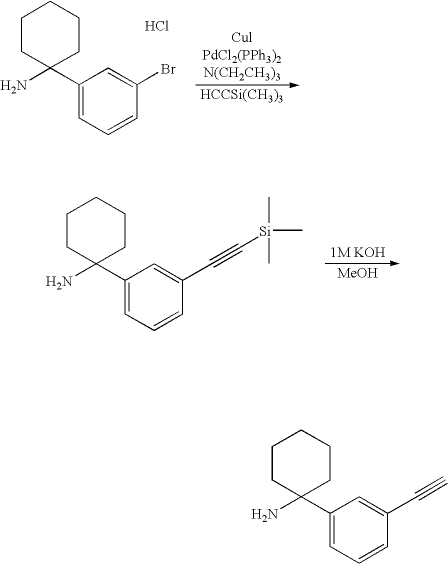

1-(3-Bromo-phenyl)-cyclohexylamine (Pharmacia, 1.04 g, 4.09 mmol) was free based and then dissolved in triethylamine (20 mL, 143 mol) prior to the addition of dicholorobis(triphenylphosphine) palladium(II) (0.119 g, 0.170 mmol) and copper iodide (0.040 g, 0.211 mmol). The reaction mixture was heated to reflux at which point trimethylsilylacetylene (0.85 mL, 6.01 mmol) was added via syringe. After refluxing for 3 h, the reaction mixture was cooled to room temperature before partitioning between EtOAc and saturated NaHCO₃ (aq). The aqueous phase was collected and extracted with EtOAc (3×). The organic phases were then collected and washed with saturated NaCl (aq), separated, dried (sodium sulfate) and concentrated under reduced pressure. The crude product was used without further purification.

The trimethylsilyl intermediate was dissolved in methanol (5 mL) and 1 N KOH (6 mL) and stirred at room temperature for 5.5 h. The reaction mixture was then partitioned between EtOAc and saturated NaHCO₃ (aq). The organic layer was separated, dried (sodium sulfate), and concentrated under reduced pressure. The residue was purified by flash chromatography (5% MeOH, 94.5% CHCl₂, 0.5% NH₄OH) yielding the desired amine (0.35 g, 31%): mass spec (Cl) 183.1 (M-16).

### EXAMPLE 44

### PREPARATION OF (1S, 2R)-N-[1-(3,5-DIFLUOROBENZYL)-3-[1-(3-(ETHYNYLPHENYL)CYCLOHEXYLAMINO]-2-HYDROXYPROPYL}ACETAMIDE

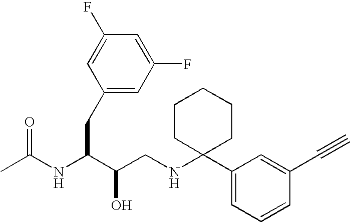

The product from EXAMPLE 43 was converted into the above titled product using methods described in EXAMPLE 22. Mass spectrometric analysis: (Cl) 441.2 (M+H).

### EXAMPLE 45

### PREPARATION OF (1S,2R)-N-(1-(3,5-DIFLUOROBENZYL)-3-{1-[3-(2,2-DIMETHYLPROPYL)PHENYL]CYCLOHEXYLAMINO)-2-HYDROXYPROPYL)ACETAMIDE

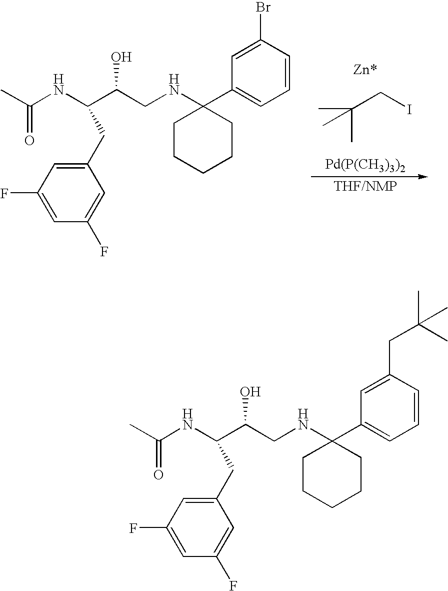

The desired product is prepared using methods that are analogous to others described in the application. Mass spec: (Cl) 487.2 (M+H), 509 (M+Na).

The Compounds Of The Present Invention That Comprise Cyclohexyl Moieties Can Be Synthesized According The General Schemes Found Below And Within Examples 32 Through 35.

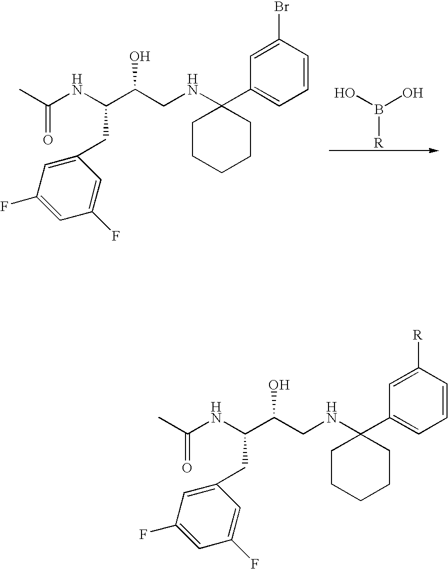

### EXAMPLE 46

### N-(1S, 2R)-(1-(3,5-DIFLUORO-BENZYL)-2-HYDROXY-3-{1-[3-(4-METHYL-THIOPHEN-2-YL)-PHENYL]-CYCLOHEXYLAMINO}-PROPYL)-ACETAMIDE

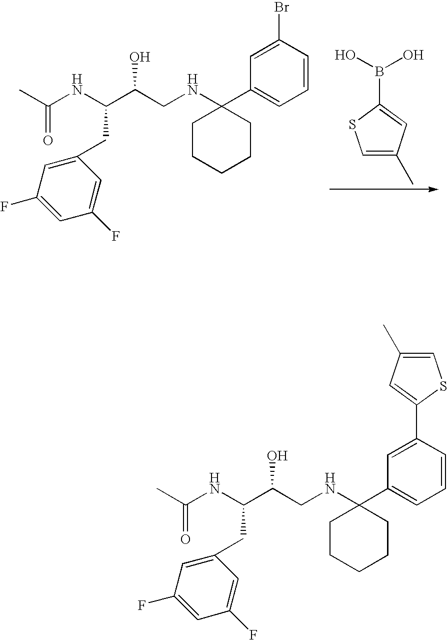

Palladium acetate (Pd(OAc)₂) (0.82 mg, 10 mol. wt. %) and Biphenyl-2-yI-di-tert-butyl-phosphane (2.16 mg, 20 mol. wt. %) was added to the reaction vessel (Vessel 1). N-(1S, 2R)-[3-[1-(3-Bromo-phenyl)-cyclohexylamino]-1-(3,5-difluoro-benzyl)-2-hydroxy-propyl]-acetamide (0.09075 mM) was placed in a separate reaction vessel (Vessel 2) and dissolved in 200 mL DME. 4-Methylthiophene-2-boronic acid and Potassium Fluoride (KF) (3 eq., 6.33 mg) were added to a separate reaction vessel and dissolved in 200 μL DME (Vessel 3). Solvents in Vessels 2 and 3 were added to Vessel 1 under nitrogen. Vessel 1 was stirred over night at room temperature. The reaction was then concentrated down by vacuum. The crude material was then purified by Prep-HPLC. The product fractions were collected and concentrated down by vacuum. MS (ESI+) for C₂₉H₃₄F₂N₂O₂S m/z 513.0 (M+H)⁺

### EXAMPLE 47

### ADDITIONAL COMPOUNDS

All compounds in EXAMPLE 1 (Exemplary Formula (I) compounds) can be essentially synthesized according to the same procedure as that used for synthesizing N-(1S,2R)-(1-(3,5-Difluoro-benzyl)-2-hydroxy-3-{1-[3-(4-methyl-thiophen-2-yl)-phenyl]-cyclohexylamino)-propyl)-acetamide; 4-methylthiophene-2-boronic acid may be replaced by other reagents as known in the art.

### EXAMPLE 48

### [3-[1-(3-BROMO-PHENYL)-CYCLOHEXYLAMINO]-1-(3,5-DIFLUORO-BENZYL)-2-HYDROXY-PROPYL]-CARBAMIC ACID TERT-BUTYL ESTER

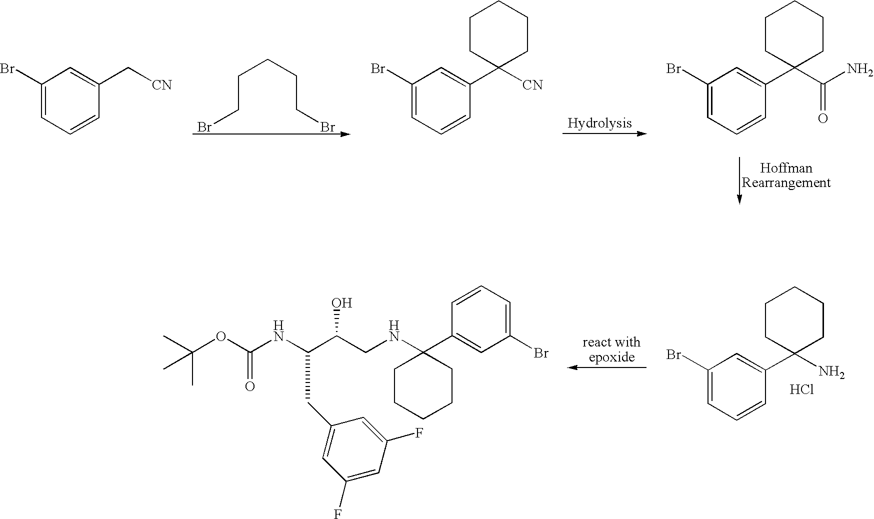

3-Bromobenzylnitrile was obtained from Kimera. Powder KOH was obtained from OxeChem. Other reagents were from Aldrich.

### Step 1: 1-(3-bromophenyl)cyclohexanecarbonitrile

To a 5 L 3-neck round-bottom flask equipped with N₂ inlet, temperature probe, addition funnel, and mechanical stirrer was added 3-bromobenzylnitrile (297 g, 1.51 mol, 1.0 eq) and THF (2.75 L). The clear solution was cooled to 0-5° C. via ice bath. KOtBu (374 g, 3.33 mol, 2.2 eq) was weighed out inside the glove box into a 200 mL round-bottom flask and added to the cold clear solution in shots. The first shot (71.1 g) was added over 30 seconds and an immediate exotherm of 9° C. was observed along with color change from clear to orange/brown solution. After waiting for 15 min for the solution to cool back down to 5.1° C., the second shot (96.0 g) was added and an exotherm of 6.5° C. was observed. After another 15 min, the third shot (100.4 g) was added and an exotherm of 5° C. was observed. After another 15 min, the fourth and final shot (106.5 g) was added and an exotherm of 3.8° C. was observed. The orange/brown solution was stirred in ice bath for 30 min upon which the solution thickened. 1,5-dibromopentane (365.5 g, 1.56 mol, 1.05 eq) was added to orange/brown mixture at such a rate to maintaining reaction temperature <15° C. The reaction changes from solution to brown slurry and the exotherm will continue to climb during addition. The addition takes ca 2 h. The addition funnel was rinsed with THF (250 mL) and added to the brown slurry. The ice bath was then removed and the slurry self-warmed to room temperature while maintaining medium agitation. A sample of the slurry was pulled after 1 h of stirring. GC indicated completion with only excess 1,5-dibromopentane and product. The light brown slurry was then filtered over a pad of celite to remove salts. The cake was rinsed with THF (ca 2 L) until clear. Ice (ca 1 L in volume) was then added to the burgundy filtrate and stirred at room temperature overnight. The mixture was then concentrated to remove THF and the resultant biphasic brown mixture was extracted with EtOAc and saturated NaCl solution. The orange organic layers were dried with anhydrous sodium sulfate, filtered and rinsed with EtOAc. The orange filtrate was then concentrated to dryness yielding a red oil. EtOAc (100 mL) was added to redissolve oil. While stirring at medium speed, heptane (2 L) was added over 1-2 min upon which burgundy oil sticks to bottom and sides of flask. The yellow solution was then carefully decanted away from the sticky oil and concentrated to dryness yielding light orange oil (379.7 g, 95% yield). GC of the light orange oil indicated excess 1,5-dibromopentane (2.8 area %), product (95.3 area %), and 7 other peaks having less than 0.5 area % (total =1.9 area %).

GC Conditions: 15 m DB5 0.25×0.25 micron; Init. Temp.=75° C., Init. Time=5 min, Rate=15° C./min, Final Temp.=275° C., Final Time=2 min, InJ. Temp.=275° C., Det. Temp.=250° C.; 1,5-dibromopentane room temperature=6.35 min, Prod. room temperature=13.47 min.

¹H NMR (400 MHz, CDCl₃); δ 7.62 (s, 1H), 7.45 (d, 2H), 7.26 (t, 1H), 2.14 (d, 2H), 1.74-1.88 (m, 6H), 1.26-1.29 (m, 2H). ¹³C NMR (100.6 MHz, CDCl₃); δ 143.63, 130.98, 130.40, 128.73, 124.41, 122.94, 122.07, 44.14, 37.23, 24.82, 23.46.

### Step 2: 1-(3-bromophenyl)cyclohexanecarboxamide

With overhead stirrer, a mixture of crude product from step 1, above, (380 g, 1207 mmol), powdered KOH (720 g) and t-BuOH (2.5 L) was heated at reflux overnight. See, for example, Hall, J. H., Gisler, M., J. Org. Chem. 1976, 41, 3769-3770. If deemed complete by GC analysis, it was cooled with ice-water (cool slowly to avoid shock to the glass), quenched with ice-water (1500 mL). The quenched mixture was then extracted with MTBE (3.5 L+1.5 L). MTBE layers were concentrated to a yellow solid, 390 g.

GC Conditions: 15 m DB5 0.25×0.25 micron; Init. Temp.=75° C., Init. Time=5 min, Rate=15° C./min, Final Temp.=275° C., Final Time=2 min, InJ. Temp.=275° C., Det. Temp.=250 ° C.; Product; room temperature=15.3 min.

### Step 3: 1-(3-bromophenyl)cyclohexanamine hydrochloride

The product from step 2, above (189 g, 603 mmol) was suspended in warmed t-BuOH (1140 mL) at −35° C., 3N NaOH (570 mL, 2.8 equiv.) was added. The reaction cooled to 30° C. NaOCl (380 mL, 13.6 wt %, 1.4 equiv.) was added in one portion. The reaction mixture was cooled to 26° C., and then started to warm up. Ice was directly added to the mixture to control the temperature <35° C. A total of 300 g of ice was used. The heat generation stopped after 15 min. All solids dissolved at that point. Assayed organic layer at 30 min, GC indicated completion. The mixture was extracted with 1100 mL of MTBE. The organic layer was combined with the organic layer of a parallel run of the same scale, and filtered to remove some white precipitate (likely urea side product). The aqueous layers were extracted with 300 mL of MTBE. The combined MTBE layers (ca. 5 L) was treated with 150 mL of conc. HCl (1.8 mol), stirred for 4h, cooled to 0° C. and filtered. The white solid was dried at 50° C. yielding a first crop of 180 g (52%) of material. The filtrate was treated with NaOH and NaHSO₃ to pH>12. The organic layer was concentrated to an oil. This oil was dissolved in 1 L of MTBE and treated with 75 mL of conc. HCl, cooled, filtered and dried yielding 140 g (40%) of the desired product Anal. Calc'd for C₁₂H₁₆BrN.HCl: C, 49.59; H, 5.90; N, 4.82; Br, 27.49; Cl, 12.20; Found: C, 50.34; H, 6.23; N, 4.70; HRMS calculated for C₁₂H₁₆BrN⁺253.0467, found 253.0470.

GC Conditions: 15 m DB5 0.25×0.25 micron; Init. Temp.=75° C., Init. Time=5 min, Rate=15° C./min, Final Temp.=275° C., Final Time=2 min, InJ. Temp.=275° C., Det. Temp.=250° C.; Product retention time=12.9 min.

### Step 4: tert-butyl-(1S,2R)-3-([1-(3-bromophenyl)cyclohexyl]amino}-1-(3,5-difluorobenzyl)-2-hydroxypropylcarbamate

The product from step 3, above (90 g, 310 mmol, 1.5 eq) was converted into a free base in 1000 mL of MTBE/400 mL of 2 N NaOH. MTBE layer was separated, washed with brine. Aqueous layers were back extracted with 400 mL of MTBE. Combined MTBE layer was concentrated (theoretical 78.3 g) yielding the free base.

61.7 g of the epoxide (206 mmol, 1 eq., FW 299.3) and the above free were suspended in (warm) 320 mL t-BuOH. A mantle and thermo/probe was used to heat the stirring mixture to 80° C. at 5° C./h ramp overnight. The mixture was concentrated on rotovap with 20° C. condenser. The resulting oil was dissolved in MTBE (1 N), washed with 1 N HCl (200 mL, then 100 mL×5) (to contain the product from this step, the first wash was quickly separated to avoid crash out). Aqueous layer was sequentially back-extracted with MTBE (200 mL). The MTBE layer was stirred with 1 N NaOH (500 mL) for 30 min, then separated. The layer was washed with brine and then concentrated to dryness. The product was recrystallized in MTBE/Heptane (150/900 mL), and then filtered at 0° C. and washed with heptane (150 mL×2), dried at 45° C., yielding 95.3 g (83.5%).

The HCl washes (suspension) were basified with 50% NaOH (ca. 50 g), extracted with MTBE (400 mL+200 mL). The MTBE layer was treated with conc. HCl (15 mL). The resulting suspension was cooled and filtered yielding the unreacted starting amine, the product from step 3, above, 31.3 g (52%).

HPLC conditions: Luna C18(2), 3 micron, min, 80:20 0.1% TFA in MeOH/0.1% TFA in water; 10 min, Product retention time=2.0 min.

### EXAMPLE 49

### SUBSTITUTED UREAS AND CARBAMATES UREAS AND CARBAMATES

Scheme 10 sets forth a general method used in the invention to prepare the appropriate compounds of formula (I). All reactions were run in 4-mL vials. 0.07 mmol of the starting amine is placed in each reaction vial. Next, 0.28 mmol (4 equiv.) of diisopropylethylamine is added in each vial. 0.077 mmol (1.1 equiv.) of each isocyanate or chloroformate is then added into the reaction vial. Finally, the starting reagents are dissolved in 1.5 mL of dichloromethane. Each reaction was run overnight at room temperature. LC/MS analysis for each reaction was performed via an Agilent 1100 HPLC, utilizing a Thermo-Hypersil C18 50×3 mm 5 micron column, coupled to a Thermo-Finnigan LCQ MS. Final purification of each product was performed via a Varian Pro Star Preparative HPLC utilizing a Phenomenex C18 60×21.2 mm 5 micron column.

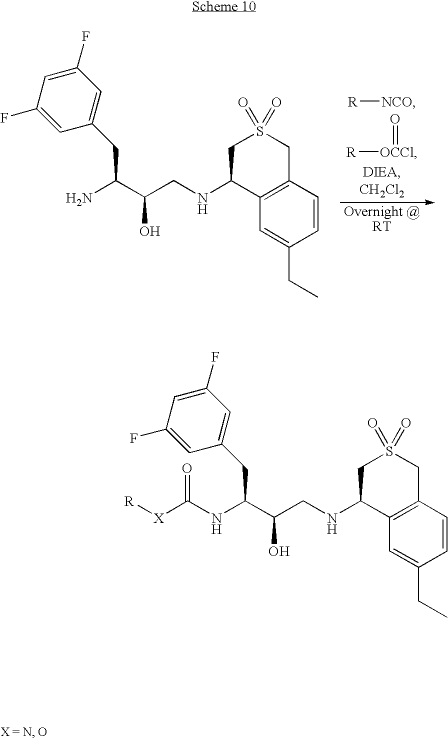

Alternatively, scheme 11 sets forth a general method to prepare appropriate compounds of formula (I). A protected amine is reacted with phosgene or phosgene equivalent such as triphosgene to generate an isocyanate that is subsequently reacted with an appropriate nucleophile. Finally, removal of the protecting group and purification by preparative HPLC will provide amines of formula (I).

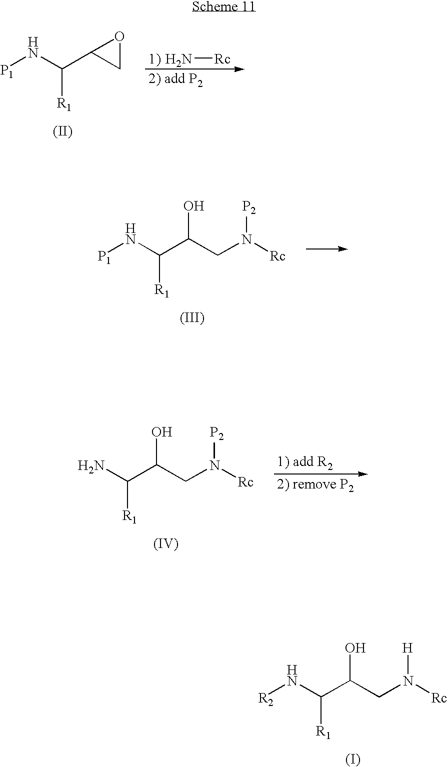

The general synthesis of compounds (I) are shown in the above Scheme. Chiral epoxides (II), which were derived from amino acids and were known in the art (see Luly, J. R. et al. J. Org. Chem. 1987, 52, 1487; Tucker, T. J. et al. J. Med. Chem. 1992, 35, 2525), were treated with 1.5-5 equivalents of primary amine H₂N—R_{c} in an alcoholic solvent, such as ethanol, isopropanol, or sec-butanol to effect ring opening of the epoxide. A preferred embodiment is to perform this reaction at elevated temperatures from 40° C. to reflux. A more preferred embodiment is to perform this reaction at reflux in isopropanol.

The resulting amino alcohol was then protected with capping group P₂. Appropriate protecting groups such as tert-butoxycarbonyl (Boc) or benzyloxycarbonyl (Cbz) may be introduced via treatment with the appropriate anhydride or carbamoyl chloride as known in the art in order to provide compounds of type (III). It is preferred to select protecting groups P₂ which may be orthogonally removed independently from P₁.

When an amino protecting group is used when preparing the inventive compounds, but no longer needed, it is removed by methods well known to those skilled in the art. By definition the amino protecting group must be readily removable as is known to those skilled in the art by methods well known to those skilled in the art. Suitable amino protecting groups include t-butoxycarbonyl, benzyloxycarbonyl, formyl, trityl, acetyl, trichloroacetyl, dichloroacetyl, chloroacetyl, trifluoroacetyl, difluoroacetyl, fluoroacetyl, 4-phenylbenzyloxycarbonyl, 2-methylbenzyloxycarbonyl, 4-ethoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxycarbonyl, cyclopentanyloxycarbonyl, 1-methylcyclopentanyloxycarbonyl, cyclohexanyloxycarbonyl, 1-methylcyclohexanyloxycabonyl, 2-methylcyclohexanyloxycarbonyl, 2-(4-toluylsulfonyl)ethoxycarbonyl, 2-(methylsulfonyl)ethoxycarbonyl, 2-(triphenylphosphino)ethoxycarbonyl, fluorenylmethoxycarbonyl, 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxyl)benzyloxycarbonyl, isobornyloxycarbonyl and 1-piperidyloxycarbonyl, 9-fluorenylmethyl carbonate, —CH—CH═CH₂, phenyl-C(═N—)—H, and the like.

Suitable means for removal of the amine-protecting group depends on the nature of the protecting group. Those skilled in the art, knowing the nature of a specific protecting group, know which reagent is preferable for its removal. For example, it is preferred to remove the preferred protecting group, Boc, by dissolving the protected material in a trifluoroacetic acid/dichloromethane mixture.

The addition of the group R₂ may be achieved by a variety of methods known in the art, depending on the nature of R₂. If R₂ is an arylsulfonyl group, the conversion may be achieved through use of a sulfonyl chloride. In the case of R₂=carbamoyl, the use of carbamoyl chlorides or carbamoyl anhydrides would afford the final compounds (I). Introduction of R₂=urethane may be achieved by treatment with the corresponding carbamyl chloride. Alternatively, treatment of amine (IV) with phosgene or phosgene equivalent (such as triphosgene) in the presence of a tertiary amine (such as triethylamine) to form the isocyanate, then condensation with an appropriate amine would also form the urethane. Removal of the protecting group P₂ by methods known in the art would then afford (I). Formation of R₂=amido may be performed by use of the appropriate carboxylic acid. The formation of the amide bond from the free amine and a given carboxylic acid may be performed by a variety of methods known in the art, such as with the use of BOP reagent (benzotriazolyl-N-hydroxytris(dimethylamino)phosphonium hexafluorophosphate) (Castro, B. et al. Tetrahedron Lett. 1975, 1219) or EDC (1-(3-dimethylaminopropyl)-3-ethylcarbodiimide hydrochloride) (Kimura, T. et al. Biopolymers 1981, 20, 1823). The synthesis of R₂=thioamido may be achieved from the amido compounds and sulfur-introducing agents known in the art, such as phosphorus pentasulfide or Lawesson's reagent (2,4-bis(4-methoxyphenyl)-1,3-dithia-2,4-diphosphetane-2,4-disulfide).

The compounds of the invention may contain geometric or optical isomers as well as tautomers. Thus, the invention includes all tautomers and pure geometric isomers, such as the E and Z geometric isomers, as well as mixtures thereof. Furthermore, the invention includes pure enantiomers and diasteriomers as well as mixtures thereof, including racemic mixtures. The individual geometric isomers, enantiomers, or diasteriomers may be prepared or isolated by methods known in the art.

### EXAMPLE 50

### PHENACYL-2-HYDROXY-3-DIAMINOALKANES AND BENZAMIDE-2-HYDROXY-3-DIAMINOALKANES

An example of one of many various processes that can be used to prepare the compounds of the invention is set forth in Scheme 12.

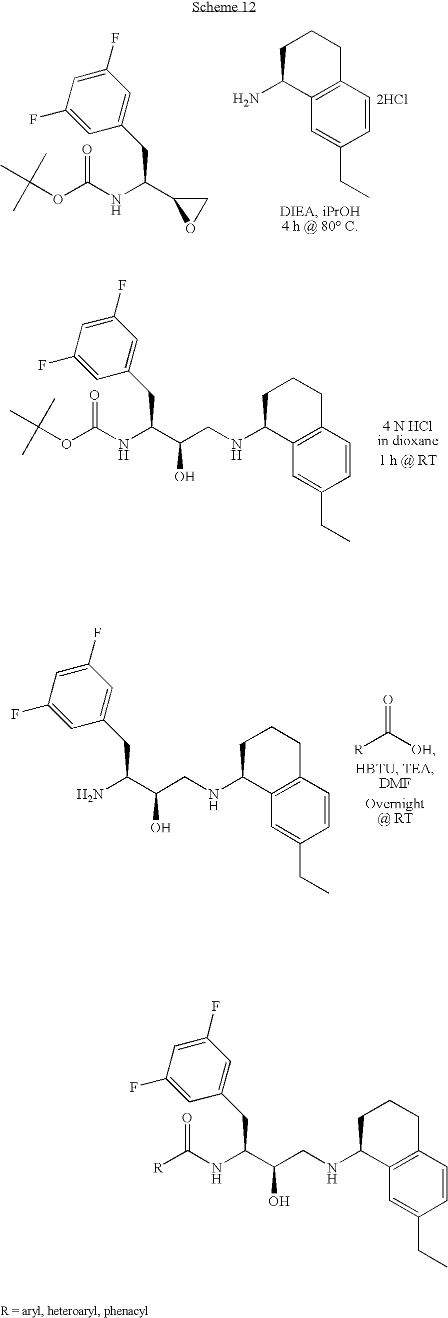

The epoxide opening in the first step in Scheme 12 was carried out with a 1:1 molar ratio of the erythro epoxide to the bicyclic C-terminal piece in a 20-mL reaction vial. Four equivalents of diisopropylethylamine were then added to the vial. Next, add 10 mL of isopropanol. Heat this reaction to 80° C. and let run for 4 h. The isopropanol and diisopropylethylamine were dissolved using a nitrogen stream.

The Boc-group deprotection in the second step was accomplished by using 3 equivalents of 4 N HCl in dioxane with respect to the amount of starting material. This reaction was run at room temperature for 1 h. The dioxane was then dissolved under a nitrogen stream.

Each reaction in the third step was run in a 4-mL vial. 0.07 mmol of the starting amine was placed in each reaction vial. Next, 0.14 mmol (2 equiv.) triethylamine was added in each vial. Then, 0.077 mmol (1.1 equiv.) of the carboxylic acid is added into the reaction vial. The starting reagents were then dissolved in 1.5 mL of DMF. Finally, 0.077 mmol (1.1 equiv.) of HBTU, dissolved in 0.5 mL DMF, is added. Each reaction was run overnight at room temperature. LC/MS analysis for each reaction was performed via an Agilent 1100 HPLC, utilizing a Thermo-Hypersil C18 50×3 mm 5 micron column, coupled to a Thermo-Finnigan LCQ MS. Final purification of each product was performed via a Varian Pro Star Preparative HPLC utilizing a Phenomenex C18 60×21.2 mm 5 micron column.

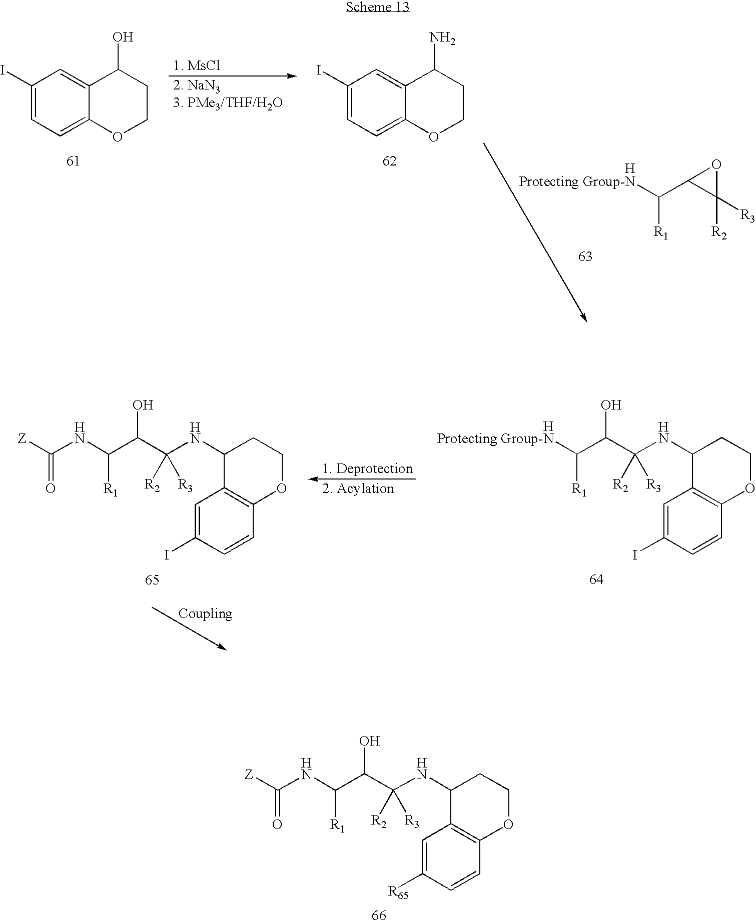

Scheme 13 illustrates the preparation of compounds using the readily obtainable 6-iodo-chroman-4-ol (61) as a starting material (see Synthesis, 1997, 23-25). One skilled in the art will recognize that there are several methods for the conversion of the alcohol functionality to the desired amino compounds 62. In Scheme 13 the alcohol 61 is first activated with methane sulfonyl chloride and the resulting mesylate displaced with sodium azide NaN₃. Alternative methods for the conversion of an alcohol to an azide are well known to one skilled in the art. The resulting azide is subsequently reduced using trimthylphosphine in a mixture of THF and water. One skilled in the art will recognize that there are several methods for the reduction of an azide to the corresponding amine. For examples, see Larock, R. C. in Comprehensive Organic Transformations, Wiley-VCH Publishers, 1999. This reduction of the azide produces a mixture of enantiomers of the amine 62. This enantiomeric mixture can be separated by means known to those skilled in the art such as low temperature recrystallization of a chiral salt or by chiral preparative HPLC, most preferably by HPLC, employing commercially available chiral columns.

The resulting amine 62 is used to open the epoxide 63 yielding the protected (6-iodo-3,4-dihydro-2H-chromen-4-yl)amino propyl carbamate 64. Suitable reaction conditions for opening the epoxide 63 include running the reaction in a wide range of common and inert solvents. C₁-C₆ alcohol solvents are preferred and isopropyl alcohol most preferred. The reactions can be run at temperatures ranging from 20-25° C. up to the reflux temperature of the alcohol employed. The preferred temperature range for conducting the reaction is between 50° C. and the refluxing temperature of the alcohol employed.

The protected iodo-chromen 64 is deprotected to the corresponding amine by means known to those skilled in the art for removal of amine protecting groups. Suitable means for removal of the amine protecting group depend on the nature of the protecting group. Those skilled in the art, knowing the nature of a specific protecting group, know which reagent is preferable for its removal. For example, it is preferred to remove the preferred protecting group, Boc, by dissolving the protected iodo-chroman in a trifluoroacetic acid/dichloromethane (1/1) mixture. When complete the solvents are removed under reduced pressure yielding the corresponding amine (as the corresponding salt, i.e. trifluoroacetic acid salt) which is used without further purification. However, if desired, the amine can be purified further by means well known to those skilled in the art, such as for example recrystallization. Further, if the non-salt form is desired that also can be obtained by means known to those skilled in the art, such as for example, preparing the free base amine via treatment of the salt with mild basic conditions. Additional Boc deprotection conditions and deprototection conditions for other protecting groups can be found in T. W. Green and P. G. M. Wuts in Protecting Groups in Organic Chemistry, John Wiley and Sons, 1999.

The amine is then reacted with an appropriately substituted amide forming agent Z-(CO)—Y to produce coupled amides 65 by nitrogen acylation means known to those skilled in the art. Nitrogen acylation conditions for the reaction of amine with an amide forming agent Z-(CO)—Y are known to those skilled in the art and can be found in R. C. Larock in Comprehensive Organic Transformations, VCH Publishers, 1989, p. 981, 979, and 972. Y comprises —OH (carboxylic acid) or halide (acyl halide), preferably chlorine, imidazole (acyl imidazole), or a suitable group to produce a mixed anhydride.

The acylated iodo-chromen 65 is coupled with an appropriately functionalized organometallic R₆₅M yielding compounds of formula 66 using conditions known to those skilled in the art. One skilled in the art will recognize that there are several methods for coupling various alkyl and aryl groups to an aromatic iodide. For examples, see L. S. Hegedus Transition Metals in the Synthesis of Complex Organic Molecules, University Science, 1999.

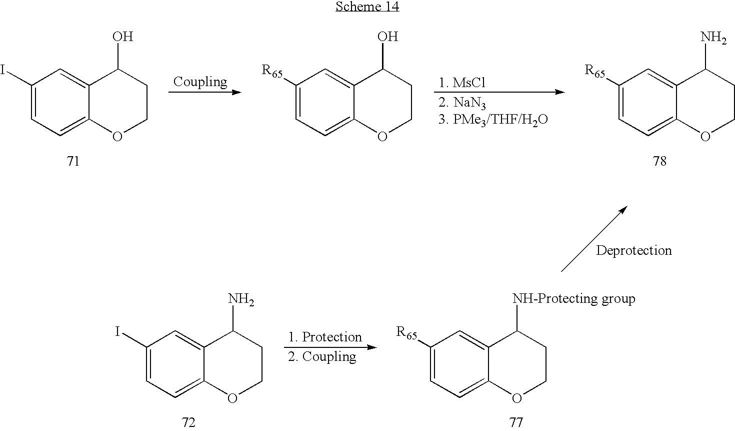

Amines of formula (78) can be prepared by coupling the appropriately functionalized organometallic to 6-iodo-chroman-4-ol 71 or to the appropriately protected iodo-amino chroman 77, as shown in Scheme 14. The chemistry from this point forward follows the generalizations described for Scheme 13.

Generally, the protection of amines is conducted, where appropriate, by methods known to those skilled in the art. See for example, Protecting Groups in Organic Synthesis, John Wiley and sons, New York, N.Y., 1981, Chapter 7; “Protecting Groups in Organic Chemistry”, Plenum Press, New York, N.Y., 1973, Chapter 2. When the amino protecting group is no longer needed, it is removed by methods known to those skilled in the art. By definition the amino protecting group must be readily removable. A variety of suitable methodologies are known to those skilled in the art; see also T. W. Green and P. G. M. Wuts in Protective Groups in Organic Chemistry, John Wiley and Sons, 1991. Suitable amino protecting groups include t-butoxycarbonyl, benzyl-oxycarbonyl, formyl, trityl, phthalimido, trichloro-acetyl, chloroacetyl, bromoacetyl, iodoacetyl, 4-phenylbenzyloxycarbonyl, 2-methylbenzyloxycarbonyl, 4-ethoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 2-(4-xenyl)isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxy-carbonyl, cyclopentanyloxycarbonyl, 1-methylcyclo-pentanyloxycarbonyl, cyclohexanyloxycarbonyl, 1-methyl-cyclohexanyloxycabonyl, 2-methylcyclohexanyloxycarbonyl, 2-(4-toluylsulfonyl)ethoxycarbonyl, 2-(methylsulfonyl)-ethoxycarbonyl, 2-(triphenylphosphino)ethoxycarbonyl, fluorenylmethoxycarbonyl, 2-(trimethylsilyl)ethoxy-carbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxyl)benzyloxycarbonyl, isobornyloxycarbonyl, 1-piperidyloxycarbonyl, 9-fluoroenylmethyl carbonate, —CH—CH═CH₂, and the like.

In an embodiment, the protecting group is t-butoxycarbonyl (Boc) and/or benzyloxycarbonyl (CBZ). In another embodiment, the protecting group is Boc. One skilled in the art will recognize suitable methods of introducing a Boc or CBZ protecting group and may additionally consult Protective Groups in Organic Chemistry, for guidance.

### EXAMPLE 51

### [2-(3,5-DIFLUORO-PHENYL)-1-OXIRANYL-ETHYL]-CARBAMIC ACID TERT-BUTYL ESTER

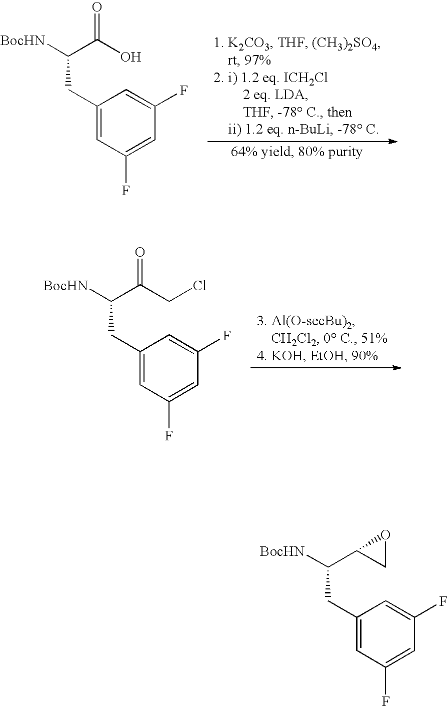

(2S)-2-[(tert-Butoxycarbonyl)amino]-3-(3,5-difluorophenyl)propionic acid methyl ester. A solution of (2S)-2-[(tert-butoxycarbonyl)amino]-3-(3,5-difluorophenyl)propionic acid (138 g, 458 mmol) was dissolved in THF (1000 mL) and cooled to 0° C. Potassium carbonate (69.6 g, 503.8 mmol) was added followed by the dropwise addition of dimethyl sulfate (45.5 mL, 480.9 mmol). The reaction was removed from the ice bath and allowed to stir at room temperature overnight after which HPLC analysis shows the complete consumption of starting material. The reaction was quenched by the addition of 10% ammonium hydroxide (150 mL). The aqueous layer was removed and extracted with ethyl acetate (500 mL). The combined organics were washed with brine (500 mL), dried over magnesium sulfate and concentrated yielding a yellow solid. The solid was recrystallized from hexanes yielding the product as an off white solid (140.3 g, 445.0 mmol, 97%).

Tert-Butyl (1 S)-3-chloro-1-(3,5-difluorobenzyl)-2-oxopropylcarbamate. A solution of LDA was prepared by adding n-BuLi (26 mL, 260 mmol) to a solution of diisopropylamine (26.3 g, 260 mmol) in THF (200 mL) at −78° C. After the addition was complete, the reaction was allowed by warm to 0° C. This light yellow solution was added dropwise to a solution of (2S)-2-[(tert-butoxycarbonyl)amino]-3-(3,5-difluorophenyl)propionic acid methyl ester (40 g, 127 mmol) and chloroiodomethane (11.1 mL, 152 mmol) keeping the temperature below −65° C. After the addition, the solution was stirred for 30 min at −78° C. n-BuLi (15 mL, 150 mmol) was added dropwise keeping the internal temperature below −62° C. The reaction was stirred for 30 min at −78° C. then quenched into 500 mL of 1 N HCl at 0° C. The product was extracted into EtOAc (500 mL), washed with brine (300 mL), dried over magnesium sulfate and concentrated. Octane (400 mL) was added to the product and the resulting solid collected by filtration and dried. The octane was cooled to −78° C. then allowed to warm until the octane melted. The resulting solid was collected and added to the previously collected solid. Drying of the combined solid gave the title compound as an off-white solid (33.9 g, 101.5 mmol, 64.5%).

Tert-Butyl (1 S, 2S)-3-chloro-1-(3,5-diflurorbenzyl)-2-hydroxypropylcarbamate. A solution of tert-butyl (1S)-3-chloro-1-(3,5-difluorobenzyl)-2-oxopropylcarbamate (67.4 g, 202 mmol) was dissolved in DCM (500 mL) and cooled to 0° C. Tri(sec-butoxy)aluminum (54.7 g, 222.1 mmol, 1.1 eq) in DCM (50 mL) was added dropwise. After stirring for 2 h at 0° C., the reaction was complete by HPLC. The reaction was quenched with 1 N HCl (750 mL) and the product extracted into ethyl acetate (2×400 mL). The combined organics were washed with brine (500 mL), dried over magnesium sulfate and concentrated yielding an oily yellow solid. Octane (300 mL) was added and the resulting solid was collected by filtration and washed with octane (100 mL). Drying overnight gave a white solid. The octane layers were collected and concentrated to ˜100 mL of volume, then placed in the freezer in the weekend to yield a second crop of the title compound (35 g, 104 mmol, 51%).

Tert-Butyl (1S)-2-(3,5-diflurorphenyl)-1-[(2S)-oxiranyl]ethylcarbamate. A solution of tert-butyl (1S, 2S)-3-chloro-1-(3,5-diflurorbenzyl)-2-hydroxypropylcarbamate in ethanol (150 mL) was cooled to 0° C. A solution of KOH in EtOH (25 mL) was added. The reaction was removed from the ice bath and stirred for 2 h. The reaction was diluted with 300 mL of water and placed into an ice bath. The resulting solid was collected by filtration and washed with cold water (100 mL). Drying overnight gave an off-white solid (6.74 g, 22.51 mmol, 90%).

### EXAMPLE 52

### 4-AMINO-6-(2,2-DIMETHYL-PROPYL)-3,4-DIHYDRO-2H-QUINOLINE-1-CARBOXYLIC ACID BENZYL ESTER

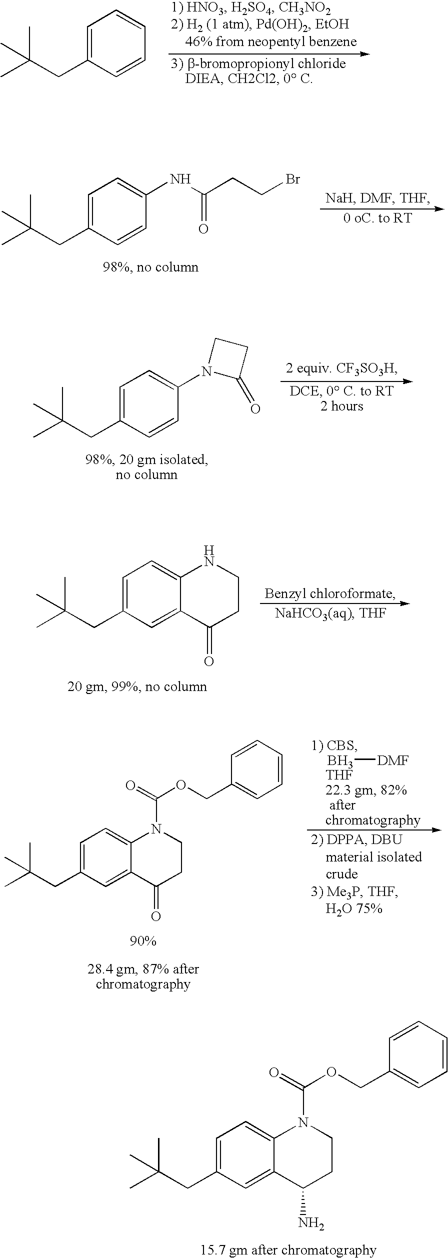

1-(2,2-Dimethyl-propyl)-4-nitro-benzene and 1-(2,2-Dimethyl-propyl)-2-nitro-benzene. To a stirred solution of concentrated sulfuric acid (13.8 mL) at 0° C. in an open flask was added concentrated HNO₃ (11.6 mL) dropwise by addition funnel. The sulfuric/nitric acid mix was then transferred to an addition funnel and added dropwise to a solution of neopentyl benzene (17.2 g, 116 mmol) in nitromethane (90 mL) stirring at 0° C. The temperature warmed to about 3° C. during the dropwise addition of the acid mixture. After complete addition, TLC in 9/1 hexanes/EtOAc showed the nitrated materials had begun forming. After warming to room temperature and stirring overnight the reaction was poured into 400 mL ice water and extracted 3×150 mL with CH₂Cl₂. The combined organics were washed 1×400 mL with H₂O, 2×400 mL with saturated NaHCO₃, and 1×400 mL with brine. The organics were dried (magnisium sulfate), filtered and concentrated to a yellow oil, which appears to be about a 1:1 mixture of regioisomers. This mixture was used crude in the subsequent reduction.

4-(2,2-Dimethyl-propyl)-phenylamine. To a stirred solution of the mixture of nitro compounds (22.4 g, 116 mmol) in 300 mL 95% EtOH was added Pearlman's catalyst (4 g). The suspension was put through a vacuum/purge cycle 3 times with hydrogen gas and then held under 1 atm H₂ overnight. TLC in 9/1 hexanes/EtOAc showed two new lower rf spots. The nitro compounds had been completely consumed. The reaction was filtered through GF/F filter paper with 95% EtOH and the filtrate concentrated. The crude material was loaded onto a Biotage 75 L column with 5/95 EtOAc/hexanes and eluted first with 5/95 EtOAc/hexanes (4 L) followed by 1/9 EtOAc/hexanes (6 L). The two regioisomeric anilines separated nicely and were concentrated yielding the undesired high rf aniline as an orange oil and the desired lower rf aniline as a tan solid (8.7 g, 46% from neopentyl benzene).

3-Bromo-N-[4-(2,2-dimethyl-propyl)-phenyl]-propionamide. To a stirred solution of the aniline (15.3 g, 93.78 mmol) in CH₂Cl₂ (300 mL) at 0° C. under nitrogen was added dimethylaniline (12.5 g, 103 mmol) followed by b-bromopropionyl chloride (17.68 g, 103 mmol). After 2 h, the reaction was diluted to 400 mL with CH₂Cl₂ and washed 3×300 mL with 2 N HCl, 3×300 mL with saturated NaHCO₃, and 1×300 mL with brine. The organics were dried (magnisium sulfate), filtered and concentrated to a white solid (27.5 g, 98%).

1-[4-(2,2-Dimethyl-propyl)-phenyl]-azetidin-2-one. To a stirred solution of DMF (115 mL) at 0° C. under nitrogen was added sodium hydride (60% oil dispersion, 4.61 g, 115 mmol). The b-bromoamide 27.5 g, 92 mmol) was then added dropwise by cannulation in 270 mL THF. Gas evolution was observed and the cooling bath was allowed to slowly melt and the reaction stirred at room temperature overnight. The white suspension was then partitioned between EtOAc (400 mL) and brine (300 mL). The organics were isolated and washed 3×300 mL with brine. The organics were dried (magnisium sulfate), filtered and concentrated to an off white solid (20 g, 100%).

6-(2,2-Dimethyl-propyl)-2,3-dihydro-1H-quinolin-4-one. To a stirred solution of the b-lactam (20.1 g, 92.5 mmol) in 300 mL dichloroethane at 0° C. under nitrogen was added triflic acid (27.76 g, 185 mmol) dropwise by syringe. The reaction was allowed to warm to room temperature and allowed to react for 4 h. Afterward, the reaction mixture was poured into 1 L of rapidly stirred 1:1 CH₂Cl₂:ice cold saturated NaHCO₃. After stirring for a few minutes the organics were isolated and the aqueous solution extracted 1×200 mL with CH₂Cl₂. The combined organics were dried (magnisium sulfate), filtered and concentrated to a yellow oil (20.1 g, 100%).

6-(2,2-Dimethyl-propyl)-4-oxo-3,4-dihydro-2H-quinoline-1-carboxylic acid benzyl ester. To a stirred solution of the tetrahydroquinolone (20.1 g, 92.5 mmol) in 300 mL CH₂Cl₂ at 0° C. under nitrogen was added DIEA (23.9 g, 185 mmol) by syringe followed by benzyl chloroformate (23.7 g, 139 mmol) dropwise by addition funnel. The reaction was allowed to warm to room temperature overnight. TLC showed near complete consumption of starting material. The reaction was transferred to a 1 L sep funnel and washed 3×300 mL with 2 N HCl and 3×300 mL with saturated NaHCO₃. The organics were dried (magnisium sulfate), filtered and concentrated to a brown oil which was loaded directly onto a Biotage 75 L column and eluted with 9/1 hexanes/EtOAc. Product containing fractions were pooled and concentrated to a pale yellow oil that solidified upon standing (28.4 g, 87% from the aniline).

6-(2,2-Dimethyl-propyl)-4-(R)-hydroxy-3,4-dihydro-2H-quinoline-1-carboxylic acid benzyl ester. To a stirred solution of the ketone (27.5 g, 79 mmol) in 79 mL THF at −25° C. (CCl4/dry ice bath) under nitrogen was added the CBS reagent (1 M in toluene, 7.9 mL, 7.9 mmol,) followed by dropwise addition of borane dimethylsulfide complex (2M in THF, 39.5 mL, 79 mmol) diluted with 95 mL THF by addition funnel, keeping the internal temperature below −20° C. After 1 h at −25° C., TLC in 3/7 EtOAc/hexanes showed some residual starting material with a new major lower rf spot dominating. The reaction was then allowed to warm to room temperature and stirred overnight. TLC showed the reaction had gone to completion. The reaction was recooled to 0° C. and quenched by addition of 190 mL MeOH via addition funnel. After removal of the cooling bath and stirring at room temperature for 2 h, the reaction was concentrated to dryness by rotovap and high vacuum and then loaded onto a Biotage 75M column with 4/1 hexanes/EtOAc and eluted. Product containing fractions were pooled and concentrated to a pale yellow oil that solidified upon standing (22.3 g, 80).

4-(S)-Azido-6-(2,2-dimethyl-propyl)-3,4-dihydro-2H-quinoline-1-carboxylic acid benzyl ester. To a stirred solution of the alcohol (22.3 g, 63 mmol) in 126 mL toluene at 0° C. under nitrogen was added DPPA (20.84 g, 75.7 mmol) neat by syringe. DBU (11.53 g, 75.7 mmol) was then added dropwise by addition funnel in 100 mL toluene. After complete addition the reaction was allowed to warm to room temperature and stir overnight. The crude reaction looked good by TLC in 4/1 hexanes/EtOAc with starting material completely consumed and a clean new higher rf spot. The reaction was reduced to about 100 mL by rotovap and was then loaded onto a Biotage 75M column with minimum CH₂Cl₂ and eluted with 5/95 EtOAc/hexanes. The product containing fractions were pooled and concentrated to a clear oil which solidified upon standing (22 g, 92%).

4-(S)-Amino-6-(2,2-dimethyl-propyl)-3,4-dihydro-2H-quinoline-1-carboxylic acid benzyl ester. To a stirred solution of the azide (22 g, 58 mmol) in 580 mL THF at room temperature under nitrogen was added H2O (1.26 g, 70 mmol) followed by trimethylphosphine (1M in toluene, 67 mL, 67 mmol) dropwise by addition funnel. After complete addition the reaction was allowed to stir overnight. TLC in EtOAc showed a trace of starting azide left with the majority of the material at the baseline. The reaction was concentrated to a yellow oil by rotary evaporation followed by high vacuum. The crude material was dissolved in EtOAc to load onto a column but a precipitate formed. The precipitate was filtered off and was shown to be not UV active on TLC and was thought to be trimethylphosphine oxide and was discarded. The crude product filtrate was loaded onto a Biotage 75M column with EtOAc and eluted with the same solvent. Product containing fractions were pooled and concentrated to a pale yellow oil (15.7 g, 77%).

### EXAMPLE 53

### 1-(3-TERT-BUTYL-5-IODO-PHENYL)-CYCLOHEXYLAMINE

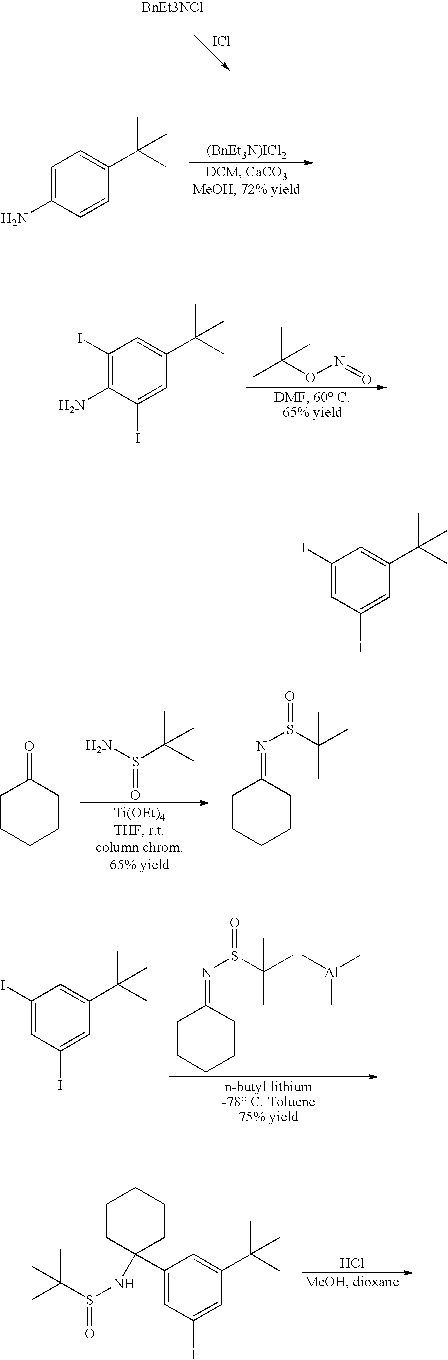

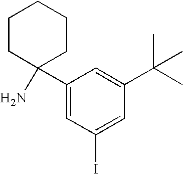

Benzytriethylammonium Dichloroiodate. To a stirred solution of ICl (146.1 g, 900 mmol) in 2 L of DCM was added BnEt₃NCl₂ (146.1 g, 900 mmol) in 1 L of water via an addition funnel over 15 min. After stirring for 30 min the layers were separated, and the organic layer was dried over magnisium sulfate, filtered, and concentrated under reduced pressure. The residue was crystallized by taking it up in minimal DCM and back adding ether to a 3:1 (DCM:ether) ratio. The material was filtered and washed with ether to yield 278 g (79.3% yield) of yellow crystals.

4-tert-Butyl-2,6-diiodo-phenylamine. To a stirred solution of t-butyl aniline (22.4 g, 150 mmol) in DCM (2 L) and MeOH (1 L) was added BnEt₃NICl₂ (122.9 g, 315 mmol) and calcium carbonate (60 g, 600 mmol). The reaction was stirred at 40° C. overnight. The reaction was filtered through a bed of celite and concentrated to 1/3 the volume. The organic phase was washed with 5% NaHSO₃, sat NaHCO₃, water, and brine. The organic layer was dried over magnisium sulfate, filtered, and concentrated yielding a red oil. The material was purified using a biotage flash 75 columns eluting with 5% EtOac in pet ether to yield 43 g (71% yield) of a dark brown oil.

1-tert-Butyl-3,5-diiodo-benzene. To a stirred solution of DMF (1.2 mL) at 60° C. was added t-butylnitrite (216 mg, 2.1 mmol) followed by 4-tert-Butyl-2,6-diiodo-phenylamine (421 mg, 1.05 mmol) in 600 uL of DMF dropwise. After stirring for 10 min, the reaction was allowed to cool, diluted with ethyl acetate (50 mL) and water (50 mL). The organic layer was washed with brine, dried over magnisium sulfate, filtered, and concentrated. The material was purified using a biotage 40 S cartridge eluting with hexanes to yield 260 mg (64% yield) of a clear oil. ¹H NMR (400 MHz, CDCl₃); δ 7.86 (t, J=1.3 Hz, 1 H), 7.65 (t, J=1.3 Hz, 2H), 1.27 (s, 9H).

### EXAMPLE 54

### 2-METHYL-PROPANE-2-SULFINIC ACID [1-(3-TERT-BUTYL-5-IODO-PHENYL)-CYCLOHEXYL]-AMIDE, AND 2-METHYL-PROPANE-2-SULFINIC ACID CYCLOHEXYLIDENEAMIDE

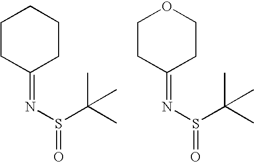

The following sulfinimines were prepared according to the method of Liu, G. et al. J. Org. Chem. 1999, 64, 1278-1284. Organolithium and Grignard additions to such ketones are described in McMahon, J. P.; Ellman, J. A. Org. Lett. 2004, 6,1645-1647.

2-Methyl-propane-2-sulfinic acid cyclohexylideneamide. To a stirred solution of cyclohexanone (1.18 g, 12 mmol) in 20 mL of THF at room temperature under nitrogen was added titanium (IV) ethoxide (4.79 g, 21 mmol) followed by 2-Methyl-propane-2-sulfinic acid amide (1.21 g, 10 mmol). After 2 h the reaction was poured into an equal volume of saturated bicarbonate stirring rapidly. The formed precipitate was filtered off by filtration through GF/F filter paper and rinse with EtOAc. The filtrate layers were separated and the aqueous layer was extracted with EtOAc. The organic layers were combined, dried over (magnisium sulfate), filtered and concentrated to a yellow oil. The material was purified by a biotage 40M cartridge eluting with hexanes:EtOac (60:40) to yield 1.25 g of a clear oil.

2-Methyl-propane-2-sulfinic acid [1-(3-tert-butyl-5-iodo-phenyl)-cyclohexyl]-amide. To a cooled (−78 ° C.) stirred solution of the 1-tert-Butyl-3,5-diiodo-benzene (3.24 g, 8.4 mmol) in dry Toluene (11 mL) was added n-butyl lithium dropwise (3.6 mL of a 2.33M solution, 8.4 mmol). The reaction was stirred at −78 for 1 h. In a separate flask the 2-Methyl-propane-2-sulfinic acid cyclohexylideneamide (805 mg, 4.0 mmol) was taken up in toluene (5 mL) cooled to −78° C. and trimethyl aluminum (2.2 mL of a 2.0M sol. In hexanes, 4.4 mmol) was added. This was stirred for 20 min and then added to the Phenyl lithium via cannulation. The reaction was stirred for 2 h at −78 ° C. and then stirred at 0° C. for 1 h. The reaction was quenched with sodium sulfate decahydrate until the bubbling stopped. Magnisium sulfate was added to the reaction and stirred for 30 min. The reaction was filtered, rinsed with EtOac and concentrated down onto silica gel. The material was purified using a biotage 40M cartridge eluting with hexanes:EtOac (60:40) to obtain 1.25 g (68% yield) of a viscous clear oil. ¹H NMR (400 MHz, CDCl₃); δ 7.61 (t, J=1.5 Hz, 1 H), 7.59 (t, J=1.5 Hz,1 H), 7.47 (t, J=1.5 Hz, 1H), 3.58 (s, 1H), 2.32-2.22 (m, 1H), 2.22-2.12 (m, 1H), 2.00-1.92 (m, 2H), 1.84-1.70 (m, 1H), 1.68-1.34 (m, 5H), 1.29 (s, 9H), 1.14 (s, 9H).

### EXAMPLE 55

### 1-(3-TERT-BUTYL-5-IODO-PHENYL)-CYCLOHEXYLAMINE HCL SALT

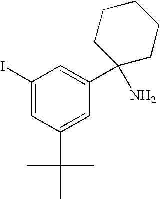

1-(3-tert-Butyl-5-iodo-phenyl)-cyclohexylamine HCl salt. To a solution of 2-Methyl-propane-2-sulfinic acid [1-(3-tert-butyl-5-iodo-phenyl)-cyclohexyl]-amide (1.25 g, 2.7 mmol) in MeOH (4 mL) was added HCl (2.7 mL of a 4M sol. in dioxane, 10.8 mmol). After stirring for 1 h the reaction was concentrated under reduced pressure to yield 1.05 g (98% yield) of a white solid. ¹H NMR (400 MHz, DMSO-d₆); δ 8.38 (s, 2H), 7.72 (d, J=1.2 Hz, 2H), 7.66 (d, J=2.0 Hz, 1H), 2.32-2.17 (m, 2H), 1.98-1.83 (m, 2H), 1.80-1.63 (m, 2H), 1.50-1.24 (m, 4H), 1.29 (s, 9H); LC retention time=3.15 min; MS (ESI) 357.6 (MH+, 100).

### EXAMPLE 56

### PROPIONIC ACID 3-(1-AMINO-CYCLOHEXYL)-PHENYL ESTERT\

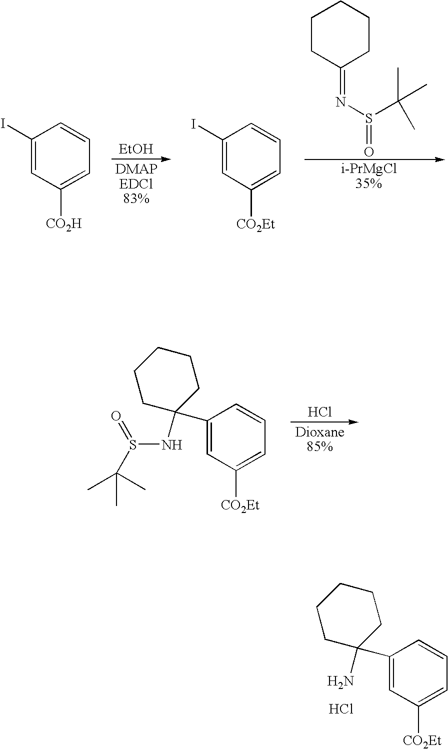

3-Iodo-benzoic acid ethyl ester. To a 250 mL round-bottom flask in a 0° C. ice bath was added 3-iodobenzoic acid (10 g, 40 mmol), EDCl (8.5 g, 44 mmol), DCM (80 mL) and allowed to stir for 10 min. To the stirred solution was added DMAP (500 mg, 4 mmol), ethanol (2.9 mL) and allowed to stir overnight. Disappearance of SM was monitored by HPLC and TLC. Reaction mixture was diluted with 1 N HCl, extracted with EtOAc, dried with magnisium sulfate, and concentrated in vacuo. Required column chromotography (10:1 Hex/EtOAc) to isolate product.

To a 50 mL round-bottom flask was added ethyl 3-iodo-benzoate (4.1 g, 15 mmol), THF (28 mL), and cooled to −20° C. Isopropylmagnesium chloride (7.5 mL, 15 mmol) was added dropwise and the reaction was stirred for 2 h at −20° C. HPLC was used to monitor disappearance of starting material. To the reaction mixture at −78° C. was then added tert-butylsulfinamide (2.0 g, 10 mmol) and the reaction allowed to warm to room temperatureand stirred overnight. The reaction was worked up with H2O, EtOAc, dried in vacuo and purified (2:1 Hex/EtOAc).

3-[1-(2-methylpropane-2-sulfinylamino0-cyclohexyl]-benzoic acid ethyl. To a 50 mL round-bottom flask was added the tert-butyl sulfinimine (933 mg, 2.65 mmol), EtOH (13 mL) and HCl/doxane (3.3 mL) and allowed to stir at room temperature for 30 min. Disappearance of starting material was monitored by HPLC. The reaction was concentrated and the resulting was dried, rinsed with Et₂O (2×), and dried again in vacuo. Product was a white solid. ¹H NMR (400 MHz, CDCl₃); δ 8.65 (br s, 2H), 8.19 (s, 1H), 7.98 (d, J=7.9 Hz, 1H), 7.95 (d, J=7.9 Hz, 1H), 7.37 (d, J=7.9 Hz, 1H), 4.38 (q, J=7.2 Hz, 2H), 2.27-2.16 (m, 2H), 2.16-2.07 (m, 2H), 1.86-1.72 (m, 2H), 1.40 (t, J=7.2 Hz, 3H); LC retention time=2.66 min; MS (ESI) 231.0 (M-NH₂+, 100).

### EXAMPLE 57

### 1-(3-METHOXY-PHENYL)-CYCLOHEXYLAMINE; COMPOUND WITH GENERIC INORGANIC NEUTRAL COMPONENT

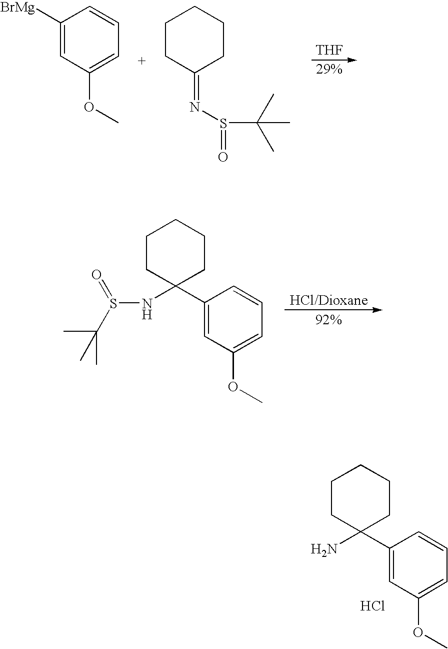

2-Methylpropane-2-sulfinic acid [1-(3-methoxyphenyl)-cyclohexyl]-amide. To a 100 mL round-bottom flask was added the 3-methoxyphenylmagnesium bromide (19 mL, 19 mmol) and THF (37 mL) and cooled to −78° C. To the mixture was then added the tert-butanesulfinamide (2.6 g, 13 mmol) and the reaction allowed to warm to room temperatureand stirred overnight. The reaction was worked up with H₂O, EtOAc, dried in vacuo and purified (3:1 Hex/EtOAc).

1-(3-Methoxyphenyl)-cyclohexylamine. To a 50 mL round-bottom flask was added the tert-butyl sulfinimine (1.16 g, 3.75 mmol), MeOH (20 mL) and HCl/dioxane (5 mL) and allowed to stir at room temperature for 30 min. Disappearance of starting material was monitored by HPLC. The reaction was concentrated yielding a solid which was dried, rinsed with Et₂O (2×), and dried again in vacuo. MS(ESI) 189.0.

### EXAMPLE 58

### 5-BROMO-2-IMIDAZOL-1-YL-BENZONITRILE

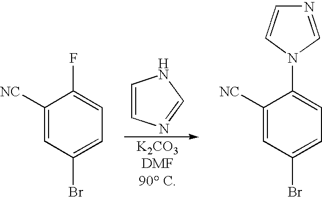

5-Bromo-2-imidazole-1-yl-benzynitrile. To a stirred solution 5-bromo-2-fluorobenzonitrile (50.0 g, 250 mmol) in DMF (300 mL) was added K₂CO₃ (69 g, 500 mmol), and then imidazole (20.0 g, 300 mmol). The reaction mixture was heated to 90° C. and stirred overnight. The reaction mixture was diluted with water and extracted with EtOAc (2×). The organic layer was washed with water (1×) and brine (1×), dried with sodium sulfate, filtered, and concentrated. Hexane was added to the resulting solid and allowed to stir for 5 min then filtered off leaving a white solid.

### EXAMPLE 59

### 4-(3-TERT-BUTYL-PHENYL)-TETRAHYDRO-PYRAN-4-YLAMINE

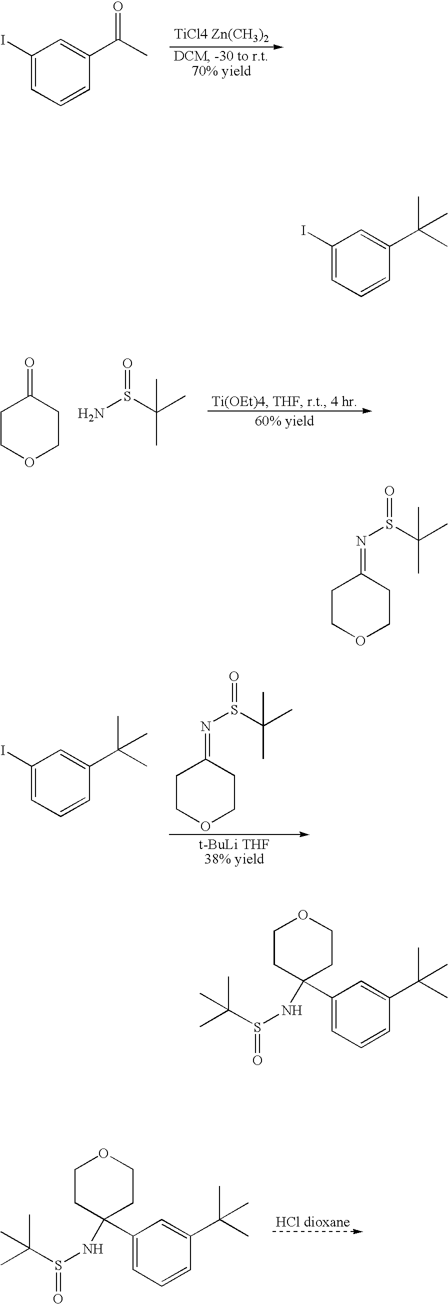

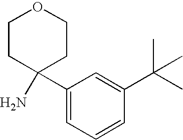

1-tert-Butyl-3-iodo-benzene. To a cooled (−40° C.) stirred solution of TiCl₄ (11 mL of a 1.0M sol in DCM, 11 mmol) in 5 mL of DCM was added dimethyl zinc (5.5 mL of a 2 N sol. in toluene, 11 mmol). After stirring for 10 min iodoacetophenone (1.23 g, 5.0 mmol) was added. After 2 h the reaction was warmed to 0° C. and stirred for an additional 1 h. The reaction was poured onto ice and extracted with ether. The organic phase was washed with water and sat NaHCO₃. The organic phase was dried over magnisium sulfate, filtered, and dried under reduced pressure. The material was distilled using a kugelrohr (80° C. at 0.1 mm) to obtain 1.0 g (76% yield) of a clear oil.

2-Methyl-propane-2-sulfinic acid (tetrahydro-pyran-4-ylidene)-amide. To a stirred solution of tetrahydro-pyran-4-one (1.2 g, 12 mmol) in 20 mL THF at room temperature under nitrogen was added titanium (IV) ethoxide (4.8 g, 21 mmol) followed by 2-methyl-propane-2-sulfinic acid amide (1.29 g, 10 mmol). The reaction was stirred at room temperature for 3 h. The reaction was quenched by pouring it into 20 mL of saturated sodium bicarbonate stirring rapidly. The formed precipitate was filtered off through GF/F filter paper and rinsed with EtOAc. The aqueous layer was washed once with EtOAc. The combined organics dried (magnisium sulfate), filtered and concentrated to a yellow oil. The material was purified using a biotage 40M cartridge eluting with hexane:ethyl acetate (60:40) to yield 1.25 g (62% yield) of a clear oil.

### EXAMPLE 60

### NH₂ REPLACEMENT OF HYDROXYL ALPHA TO THE —(CHR₁)— GROUP OF COMPOUNDS OF FORMULA (I)

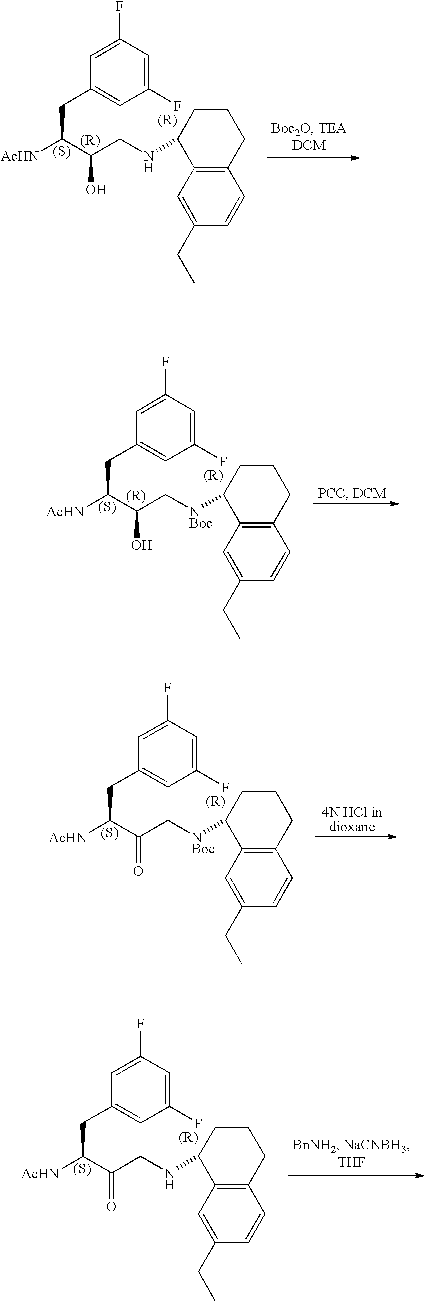

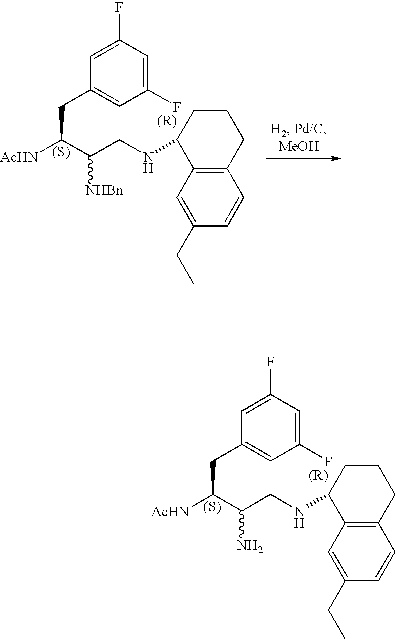

### EXAMPLE 61

### SH REPLACEMENT OF HYDROXYL ALPHA TO THE —(CHR₁)— GROUP OF COMPOUNDS OF FORMULA (I)

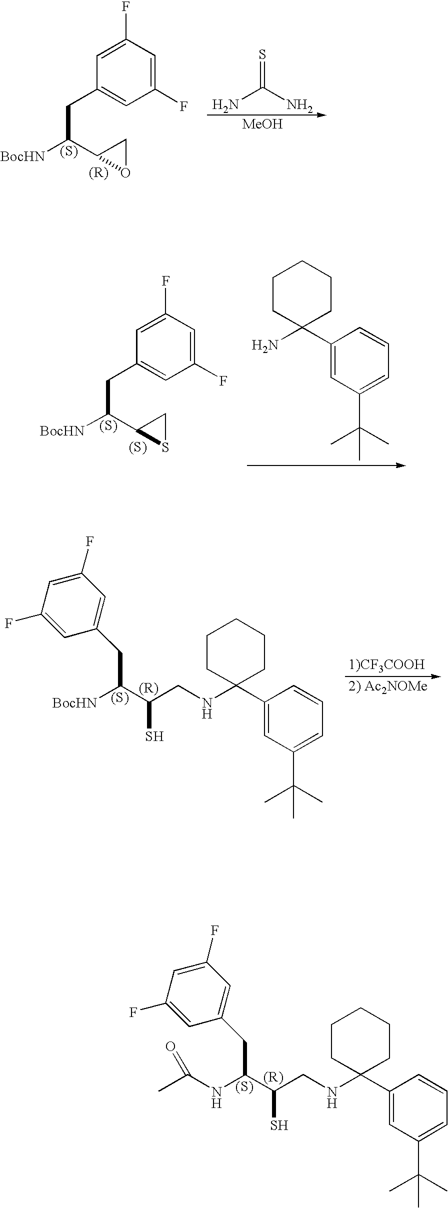

### EXAMPLE 62

### ALTERNATIVE PREPARATION OF 5-(2,2-DIMETHYL-PROPYL)-2-IMIDAZOL-1-YL-BENZYLAMINE

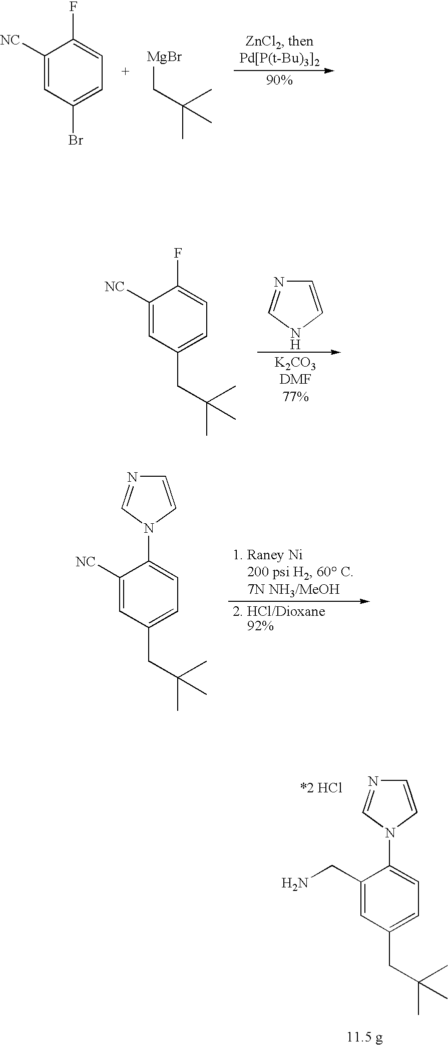

Incorporation of the neopentyl group was performed using a Negishi coupling with the neopentyl zinc species generated from the commercially available neopentylmagnesium chloride. The in situ generated neopentyl zinc reagent underwent cross-coupling reaction with the aryl bromide using the Fu catalyst at room temperature. Displacement of the aryl fluoride with imidazole occurred in DMF with heating. Reduction of the nitrile was carried out with Raney Ni. During the reduction, a significant amount of dimer was seen when Boc anhydride was used instead of ammonia. The reaction was found to proceed to completion at 200 psi of hydrogen at 60° C. Reduction of the temperature to either 20° C. or 40° C. or reducing the pressure of H₂(g) significantly reduced the rate of the reduction. The product was an oil, but treating with hydrogen chloride in dioxane gave the salt as a free flowing solid.

### Step 1: Preparation of 5-neopentyl-2-fluoro-benzonitrile

To a solution of zinc chloride (50 mL, 1.0M in diethyl ether, 50 mmol) was added neopentylmagnesium chloride (50 mL, 1.0M in THF, 50 mmol) dropwise at 0° C. During the addition, the generated magnesium salts formed a white precipitate. The reaction was removed from the ice bath and allowed to stir for 1 h then 1-bromo-2-fluorobenzonitrile (5 g, 25 mmol) was added followed by bis(tri-tert-butylphosphine) palladium (0.127 g, 0.25 mmol, 1%). The reaction began to reflux and was placed back into the ice bath. After 1 h, the reaction was diluted with 200 mL of diethyl ether and washed with 1 N HCl (2×100 mL), brine (100 mL), dried over magnesium sulfate and concentrated yielding an oily solid (4.3 g, 22 mmol, 90%).

### Step 2: Preparation of 5-neopentyl-2-imidazol-1-yl-benzonitrile

A solution of 5-neopentyl-2-fluoro-benzonitrile (4.3 g, 22.5 mmol), imidazole (1.68 g, 24.73 mmol) and potassium carbonate (6.25 g, 44.97 mmol) were stirred in DMF (50 mL) at 90° C. The reaction was stopped after 4 h and worked up, but LCMS and HNMR show starting material remaining. The crude product was resubmitted to reaction conditions and stirred overnight. The reaction was diluted with ethyl acetate (100 mL) and washed with water (2×75 mL) and brine (75 mL). The organic layer was dried over magnesium sulfate and concentrated yielding a white solid (4.16 g, 17.4 mmol, 77%); MH+240.2.

### Step 3: Preparation of 5-neopentyl-2-fluoro-benzylamine

To a solution of 5-neopentyl-2-imidazol-1-yl-benzonitrile (10.00 g, 41.79 mmol) in ammonia in methanol solution (7 N, 350 mL) was added a slurry of Raney nickel (10 mL). The reaction was sealed in a parr bomb and placed under H₂ (200 psi) then heated to 60° C. As the pressure dropped, H₂ was added to adjust the pressure to 200 psi. After 8 h, the vessel was cooled, the hydrogen was removed and the reaction was placed under N₂(g). The reaction was filtered, washed with methanol and concentrated. The resulting oil was dried for 48 h. The oil was dissolved in 50 mL of diethyl ether and 4N HCl in dioxane (32 mL) was added which caused a precipitate to form. This precipitate was collected by filtration, washed with diethyl ether (100 mL) and methylene chloride (100 mL). Drying under high vacuum gave a white solid (12.1 g, 38.3 mmol, 92%); MH+244.2.

### EXAMPLE 63

### 4-AMINO-6-(2,2-DIMETHYL-PROPYL)-3,4-DIHYDRO-2H-QUINOLINE-1-CARBOXYLIC ACID BENZYL ESTER

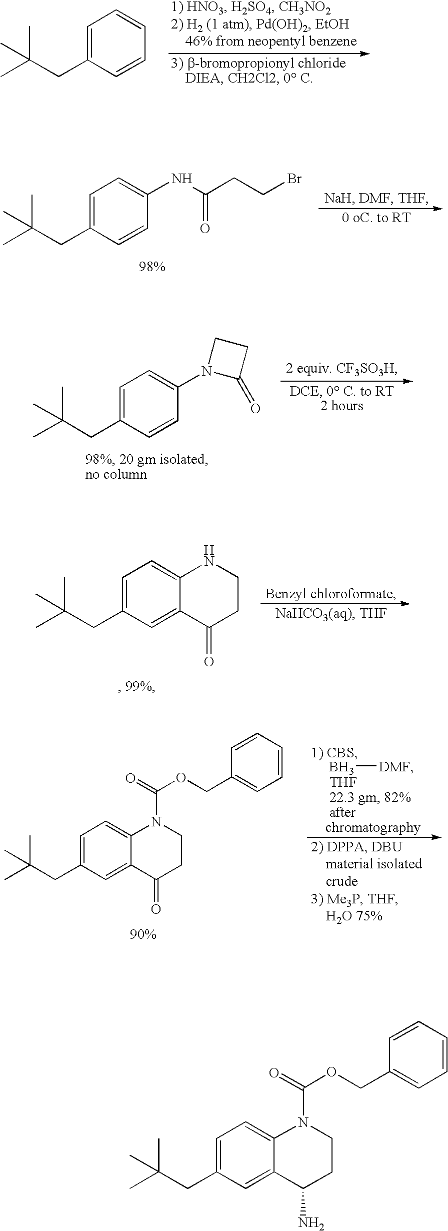

1-(2,2-Dimethyl-propyl)-4-nitro-benzene and 1-(2,2-Dimethyl-propyl)-2-nitro-benzene. To a stirred solution of concentrated sulfuric acid (13.8 mL) at 0° C. in an open flask was added concentrated HNO₃ (11.6 mL) dropwise by addition funnel. The sulfuric/nitric acid mix was then transferred to an addition funnel and added dropwise to a solution of neopentyl benzene (17.2 g, 116 mmol) in nitromethane (90 mL) stirring at 0° C. The temperature warmed to about 3° C. during the dropwise addition of the acid mixture. After complete addition, TLC in 9/1 hexanes/EtOAc showed the nitrated materials had begun forming. After warming to room temperature and stirring overnight the reaction was poured into 400 mL ice water and extracted 3×150 mL with CH₂Cl₂. The combined organics were washed 1×400 mL with H₂O, 2×400 mL with saturated NaHCO₃, and 1×400 mL with brine. The organics were dried (magnisium sulfate), filtered and concentrated to a yellow oil. This mixture was used crude in the subsequent reduction.

4-(2,2-Dimethyl-propyl)-phenylamine. To a stirred solution of the mixture of nitro compounds (22.4 g, 116 mmol) in 300 mL 95% EtOH was added Pearlman's catalyst (4 g). The suspension was put through a vacuum/purge cycle 3 times with hydrogen gas and then held under 1 atm H₂ overnight. TLC in 9/1 hexanes/EtOAc showed two new lower rf spots. The nitro compounds had been completely consumed. The reaction was filtered through GF/F filter paper with 95% EtOH and the filtrate concentrated to material corresponding to ¹H-NMR E10483_{—}007_{—}001. The crude material was loaded onto a Biotage 75L column with 5/95 EtOAc/hexanes and eluted first with 5/95 EtOAc/hexanes (4 liters) followed by 1/9 EtOAc/hexanes (6 liters). The two regioisomeric anilines separated nicely and were concentrated yielding the undesired high rf aniline as an orange oil and the desired lower rf aniline as a tan solid (8.7 g, 46% from neopentyl benzene; ¹H NMR (400 MHz, CDCl₃); δ 6.91 (d, J=6.4 Hz, 1 H), 6.61 (d, J=6.4 Hz, 1 H), 3.54 (s, 2H), 2.38 (s, 2H), 0.87 (s, 9H); LC retention time=2.89 min.

3-Bromo-N-r4-(2,2-dimethyl-propyl)-phenyl]-propionamide. To a stirred solution of the aniline (15.3 g, 93.78 mmol) in CH₂Cl₂ (300 mL) at 0° C. under nitrogen was added dimethylaniline (12.5 g, 103 mmol) followed by p-bromopropionyl chloride (17.68 g, 103 mmol). After 2 h, the reaction was diluted to 400 mL with CH₂Cl₂ and washed 3×300 mL with 2 N HCl, 3×300 mL with saturated NaHCO₃, and 1×300 mL with brine. The organics were dried (magnisium sulfate), filtered and concentrated to a white solid (27.5 g, 98%); LC retention time=4.06 min.

1-[4-(2,2-Dimethyl-propyl)-phenyl]-azetidin-2-one. To a stirred solution of DMF (115 mL) at 0° C. under nitrogen was added sodium hydride (60% oil dispersion, 4.61 g, 115 mmol). The 13-bromoamide 27.5 g, 92 mmol) was then added dropwise by cannulation in 270 mL THF. Gas evolution was observed and the cooling bath was allowed to slowly melt and the reaction stirred at room temperature overnight. The white suspension was then partitioned between EtOAc (400 mL) and brine (300 mL). The organics were isolated and washed 3×300 mL with brine. The organics were dried (magnisium sulfate), filtered and concentrated to an off white solid (20 g, 100%). The crude product was used as is in the following reaction.

6-(2,2-Dimethyl-propyl)-2,3-dihydro-1H-quinolin-4-one. To a stirred solution of the β-lactam (20.1 g, 92.5 mmol) in 300 mL dichloroethane at 0° C. under nitrogen was added triflic acid (27.76 g, 185 mmol) dropwise by syringe. The reaction was allowed to warm to room temperature. HPLC showed that the reaction had proceeded to completion after about 4 h. The reaction was poured into 1 liter of rapidly stirred 1:1 CH₂Cl₂:ice cold saturated NaHCO₃. After stirring for a few minutes the organics were isolated and the aqueous solution extracted 1×200 mL with CH₂Cl₂. The combined organics were dried (magnisium sulfate), filtered and concentrated to a yellow oil (20.1 g, 100%); LC retention time=3.87 min.

6-(2,2-Dimethyl-propyl)-4-oxo-3,4-dihydro-2H-quinoline-1-carboxylic acid benzyl ester. To a stirred solution of the tetrahydroquinolone (20.1 g, 92.5 mmol) in 300 mL CH₂Cl₂ at 0° C. under nitrogen was added DIEA (23.9 g, 185 mmol) by syringe followed by benzyl chloroformate (23.7 g, 139 mmol) dropwise by addition funnel. The reaction was allowed to warm to room temperature overnight. TLC showed near complete consumption of starting material. The reaction was transfered to a 1 N sep funnel and washed 3×300 mL with 2 N HCl and 3×300 mL with saturated NaHCO₃. The organics were dried (magnisium sulfate), filtered and concentrated to a brown oil which was loaded directly onto a Biotage 75L column and eluted with 9/1 hexanes/EtOAc. Product containing fractions were pooled and concentrated to a pale yellow oil that solidified upon standing (28.4 g, 87% from the aniline). 6-(2,2-Dimethyl-propyl)-4-(R)-hydroxy-3,4-dihydro-2H-quinoline-1-carboxylic acid benzyl ester. To a stirred solution of the ketone (27.5 g, 79 mmol) in 79 mL THF at −25° C. (CCl₄/dry ice bath) under nitrogen was added the CBS reagent (1 M in toluene, 7.9 mL, 7.9 mmol,) followed by dropwise addition of borane dimethylsulfide complex (2 M in THF, 39.5 mL, 79 mmol) diluted with 95 mL THF by addition funnel, keeping the internal temperature below −20° C. After 1 h at −25° C., TLC in 3/7 EtOAc/hexanes showed some residual starting material with a new major lower rf spot dominating. The reaction was then allowed to warm to room temperature and stirred overnight. TLC showed the reaction had gone to completion. The reaction was recooled to 0° C. and quenched by addition of 190 mL MeOH via addition funnel. After removal of the cooling bath and stirring at room temperature for 2 h, the reaction was concentrated to dryness by high vacuum and then loaded onto a Biotage 75M column with 4/1 hexanes/EtOAc and eluted. Product containing fractions were pooled and concentrated to a pale yellow oil that solidified upon standing (22.3 g, 80%); ¹H NMR (400 MHz, CDCl₃); δ 7.78 (d, J=8.2 Hz, 1 H), 7.43-7.29 (m, 5H), 7.13 (d, J=1.8 Hz, 1H), 7.03 (dd, J=8.2, 1.8 Hz, 1H), 5.24 (AB q, J=12.5 Hz, 2H), 4.75 (q, J=4.7 Hz, 1H), 4.19-4.09 (m, 1H), 3.68 (ddd, J=13.3, 9.5, 4.0 Hz, 1H), 2.46 (s, 2H), 2.14-1.97 (m, 2H), 1.71 (d, J=5.0 Hz, 1 H), 0.90 (s, 9H).

4-(S)-Azido-6-(2,2-dimethyl-propyl)-3,4-dihydro-2H-quinoline-1-carboxylic acid benzyl ester. To a stirred solution of the alcohol (22.3 g, 63 mmol) in 126 mL toluene at 0° C. under nitrogen was added DPPA (20.84 g, 75.7 mmol) neat by syringe. DBU (11.53 g, 75.7 mmol) was then added dropwise by addition funnel in 100 mL toluene. After complete addition the reaction was allowed to warm to room temperature and stir overnight. The crude reaction looked good by TLC in 4/1 hexanes/EtOAc with starting material completely consumed and a clean new higher rf spot. The reaction was reduced to about 100 mL by rotovap and was then loaded onto a Biotage 75M column with minimum CH₂Cl₂ and eluted with 5/95 EtOAc/hexanes. The product containing fractions were pooled and concentrated to a clear oil which solidifed upon standing (22 g, 92%).

4-(S)-Amino-6-(2,2-dimethyl-propyl)-3,4-dihydro-2H-quinoline-1-carboxylic acid benzyl ester. To a stirred solution of the azide (22 g, 58 mmol) in 580 mL THF at room temperature under nitrogen was added H₂O (1.26 g, 70 mmol) followed by trimethylphosphine (1 M in toluene, 67 mL, 67 mmol) dropwise by addition funnel. After complete addition the reaction was allowed to stir overnight. TLC in EtOAc showed a trace of starting azide left with the majority of the material at the baseline. The reaction was concentrated to a yellow oil by rotary evaporation followed by high vacuum. The crude material was dissolved in EtOAc to load onto a column but a precipitate formed. The precipitate was filtered off and was discarded. The crude product filtrate was loaded onto a Biotage 75M column with EtOAc and eluted with the same solvent. Product containing fractions were pooled and concentrated to a pale yellow oil (15.7 g, 77%); ¹H NMR (400 MHz, CDCl₃); δ 7.68 (d, J=8.0 Hz, 1H), 7.43-7.28 (m, 5H), 7.09 (s, 1H), 6.97 (d, J=8.1 Hz, 1H), 5.24 (AB q, J=12.5 Hz, 2H), 4.01-3.91 (m, 2H), 3.84-3.76 (m, 1H), 2.45 (s, 2H), 2.19-2.09 (m, 1H), 1.82-1.72 (m, 1H), 0.90 (s, 9H); LC retention time=3.18 min.

### EXAMPLE 64

### 1-(3-TERT-BUTYL-5-IODO-PHENYL)-CYCLOHEXYLAMINE

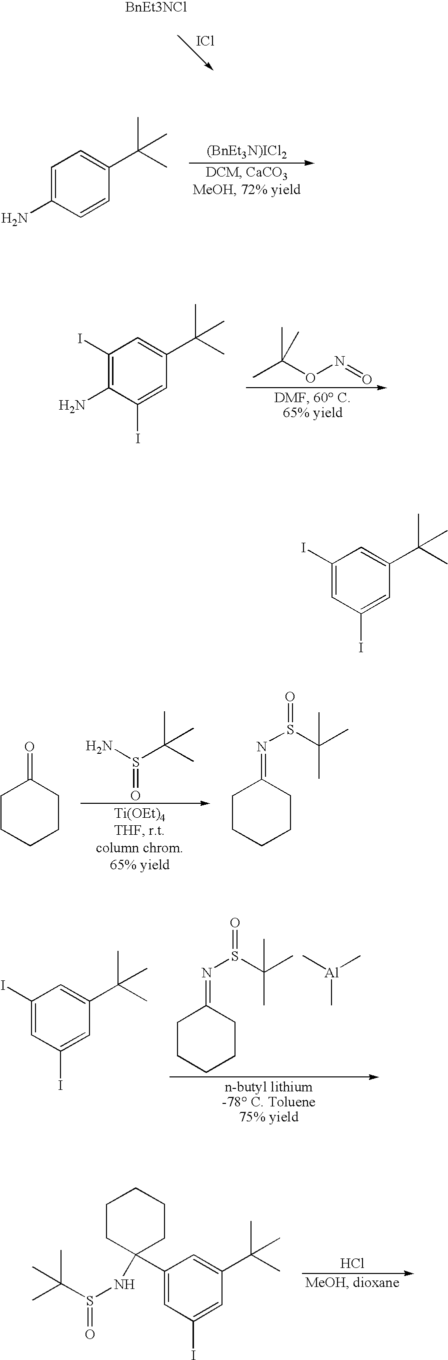

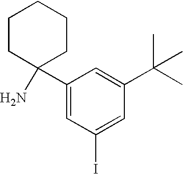

Benzytriethylammonium Dichloroiodate. To a stirred solution of ICl (146.1 g, 900 mmol) in 2 L of DCM was added BnEt₃NCl₂ (146.1 g, 900 mmol) in 1 L of water via an addition funnel over 15 min. After stirring for 30 min the layers were separated, and the organic layer was dried (magnisium sulfate), filtered, and concentrated under reduced pressure. The residue was crystallized by taking it up in minimal DCM and back adding ether to a 3:1 (DCM:ether) ratio. The material was filtered and washed with ether to yield 278 g (79.3% yield) of yellow crystals.

4-tert-Butyl-2,6-diiodo-phenylamine. To a stirred solution of t-butyl aniline (22.4 g, 150 mmol) in DCM (2 L) and MeOH (1 L) was added BnEt₃NICl₂ (122.9 g, 315 mmol) and calcium carbonate (60 g, 600 mmol). The reaction was stirred at 40° C. overnight. The reaction was filtered through a bed of celite and concentrated to 1/3 the volume. The organic phase was washed with 5% NaHSO₃, sat NaHCO₃, water, and brine. The organic layer was dried (magnisium sulfate), filtered, and concentrated yielding a red oil. The material was purified using a biotage flash 75 columns eluting with 5% EtOac in pet ether to yield 43 g (71% yield) of a dark brown oil.

1-tert-Butyl-3,5-diiodo-benzene. To a stirred solution of DMF (1.2 mL) at 60° C. was added tbutylnitrite (216 mg, 2.1 mmol) followed by 4-tert-Butyl-2,6-diiodo-phenylamine (421 mg, 1.05 mmol) in 600 uL of DMF dropwise. After stirring for 10 min, the reaction was allowed to cool, diluted with ethyl acetate (50 mL) and water (50 mL). The organic layer was washed with brine, dried over magnisium sulfate, filtered, and concentrated. The material was purified using a biotage 40 S cartridge eluting with hexanes to yield 260 mg (64% yield) of a clear oil. ¹H NMR (400 MHz, CDCl₃); δ 7.86 (t, J=1.3 Hz,1 H), 7.65 (t, J=1.3 Hz, 2H), 1.27 (s, 9H).

### EXAMPLE 65

### 2-METHYL-PROPANE-2-SULFINIC ACID [1-(3-TERT-BUTYL-5-IODO-PHENYL)-CYCLOHEXYL]-AMIDE, AND 2-METHYL-PROPANE-2-SULFINIC ACID CYCLOHEXYLIDENEAMIDE

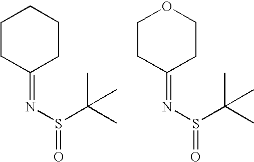

The following sulfinimines were prepared according to the method of Liu, G. et al. J. Org. Chem. 1999, 64, 1278-1284. Organolithium and Grignard additions to such ketones are described in McMahon, J. P.; Ellman, J. A. Org. Lett. 2004, 6,1645-1647.

2-Methyl-propane-2-sulfinic acid cyclohexylideneamide. To a stirred solution of cyclohexanone (1.18 g, 12 mmol) in 20 mL of THF at room temperature under nitrogen was added titanium (IV) ethoxide (4.79 g, 21 mmol) followed by 2-Methyl-propane-2-sulfinic acid amide (1.21 g, 10 mmol). After 2 h the reaction was poured into an equal volume of sat bicarb stirring rapidly. The formed precipitate was filtered off by filtration through GF/F filter paper and rinse with EtOAc. The filtrate layers were separated and the aqueous layer was extracted with EtOac. The organic layers were combined, dried over (magnisium sulfate), filtered and concentrated to a yellow oil. The material was purified by a biotage 40M cartridge eluting with hexanes:EtOac (60:40) to yield 1.25 g of a clear oil.

2-Methyl-propane-2-sulfinic acid [1-(3-tert-butyl-5-iodo-phenyl)-cyclohexyl]-amide. To a cooled (−78° C.) stirred solution of the 1-tert-Butyl-3,5-diiodo-benzene (3.24 g, 8.4 mmol) in dry Toluene (11 mL) was added n-butyl lithium dropwise (3.6 mL of a 2.33M solution, 8.4 mmol). The reaction was stirred at −78 for 1 h. In a separate flask the 2-Methyl-propane-2-sulfinic acid cyclohexylideneamide (805 mg, 4.0 mmol) was taken up in toluene (5 mL) cooled to −78° C. and trimethyl aluminum (2.2 mL of a 2.0M sol. In hexanes, 4.4 mmol) was added. This was stirred for 20 min and then added to the Phenyl lithium via cannulation. The reaction was stirred for 2 h at −78° C. and then stirred at 0° C. for 1 h. The reaction was quenced with sodium sulfate decahydrate. Magnisium sulfate was added to the reaction and stirred for 30 min. The reaction was filtered, rinsed with EtOac and concentrated down onto silica gel. The material was purified using a biotage 40M cartridge eluting with hexanes:EtOac (60:40) to obtain 1.25 g (68% yield) of a viscous clear oil. ¹H NMR (400 MHz, CDCl₃); δ 7.61 (t, J=1.5 Hz, 1 H), 7.59 (t, J=1.5 Hz, 1 H), 7.47 (t, J=1.5 Hz,1 H), 3.58 (s, 1H), 2.32-2.22 (m, 1H), 2.22-2.12 (m, 1H), 2.00-1.92 (m, 2H), 1.84-1.70 (m, 1H), 1.68-1.34 (m, 5H), 1.29 (s, 9H), 1.14 (s, 9H).

### EXAMPLE 66

### 1-(3-TERT-BUTYL-5-IODO-PHENYL)-CYCLOHEXYLAMINE HCL SALT

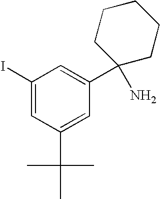

1-(3-tert-Butyl-5-iodo-phenyl)-cyclohexylamine HCl salt. To a solution of 2-Methyl-propane-2-sulfinic acid [1-(3-tert-butyl-5-iodo-phenyl)-cyclohexyl]-amide (1.25 g, 2.7 mmol) in MeOH (4 mL) was added HCl (2.7 mL of a 4M sol. in dioxane, 10.8 mmol). After stirring for 1 h the reaction was concentrated under reduced pressure to yield 1.05 g (98% yield) of a white solid. ¹H NMR (400 MHz, DMSO-d₆); δ 8.38 (s, 2H), 7.72 (d, J=1.2 Hz, 2H), 7.66 (d, J=2.0 Hz, 1H), 2.32-2.17 (m, 2H), 1.98-1.83 (m, 2H), 1.80-1.63 (m, 2H), 1.50-1.24 (m, 4H), 1.29 (s, 9H); LC retention time=3.15 min; MS (ESI) 357.6 (MH+, 100).

### EXAMPLE 67

### PROPIONIC ACID 3-(1-AMINO-CYCLOHEXYL)-PHENYL ESTERT

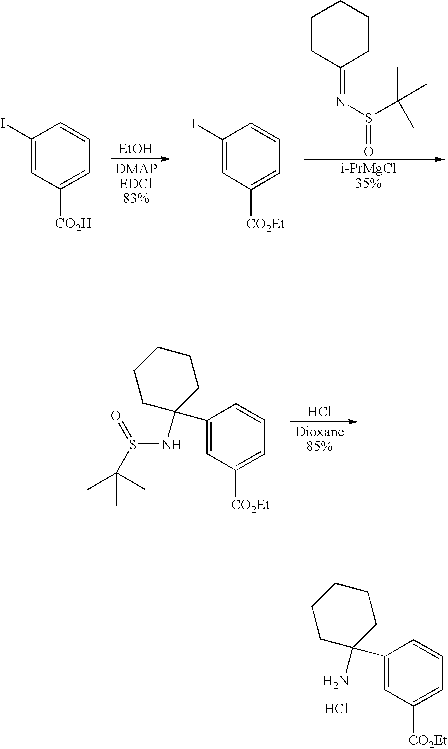

3-Iodo-benzoic acid ethyl ester. To a 250 mL round-bottom flask in a 0° C. ice bath was added 3-iodobenzoic acid (10 g, 40 mmol), EDCl (8.5 g, 44 mmol), DCM (80 mL) and allowed to stir for 10 min. To the stirred solution was added DMAP (500 mg, 4 mmol), ethanol (2.9 mL) and allowed to stir overnight. Disappearance of SM was monitored by HPLC and TLC. Reaction mixture was diluted with 1 N HCl, extracted with EtOAc, dried with magnisium sulfate, and concentrated in vacuo. Required column chromotography (10:1 Hex/EtOAc) to isolate product.

To a 50 mL round-bottom flask was added ethyl 3-iodo-benzoate (4.1 g, 15 mmol), THF (28 mL), and cooled to −20° C. Isopropylmagnesium chloride (7.5 mL, 15 mmol) was added dropwise and the reaction was stirred for 2 h at −20° C. HPLC was used to monitor disappearance of starting material. To the reaction mixture at −78° C. was then added tert-butylsulfinamide (2.0 g, 10 mmol) and the reaction allowed to warm to room temperature and stirred overnight. The reaction was worked up with H2O, EtOAc, dried in vacuo and purified (2:1 Hex/EtOAc).

3-[1-(2-methylpropane-2-sulfinylaminoO-cyclohexyl]-benzoic acid ethyl. To a 50 mL round-bottom flask was added the tert-butyl sulfinimine (933 mg, 2.65 mmol), EtOH (13 mL) and HCl/doxane (3.3 mL) and allowed to stir at room temperature for 30 min. Disappearance of starting material was monitored by HPLC. The reaction was concentrated and the resulting was dried, rinsed with Et₂O (2×), and dried again in vacuo. Product was a white solid. ¹H NMR (400 MHz, CDCl₃); δ 8.65 (br s, 2H), 8.19 (s, 1H), 7.98 (d, J=7.9 Hz, 1H), 7.95 (d, J=7.9 Hz, 1H), 7.37 (d, J=7.9 Hz, 1H), 4.38 (q, J=7.2 Hz, 2H), 2.27-2.16 (m, 2H), 2.16-2.07 (m, 2H), 1.86-1.72 (m, 2H), 1.40 (t, J=7.2 Hz, 3H); LC retention time =2.66 min; MS (ESI) 231.0 (M-NH₂+, 100).

### EXAMPLE 68

### 1-(3-METHOXY-PHENYL)-CYCLOHEXYLAMINE; COMPOUND WITH GENERIC INORGANIC NEUTRAL COMPONENT

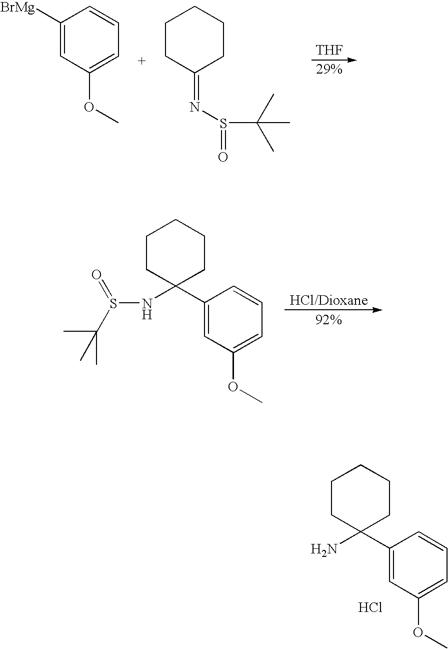

2-Methylpropane-2-sulfinic acid [1-(3-methoxyphenyl)-cyclohexyl]-amide. To a 100 mL round-bottom flask was added the 3-methoxyphenylmagnesium bromide (19 mL, 19 mmol) and THF (37 mL) and cooled to −78° C. To the mixture was then added the tert-butanesulfinamide (2.6 g, 13 mmol) and the reaction allowed to warm to room temperature and stirred overnight. The reaction was worked up with H₂O, EtOAc, dried in vacuo and purified (3:1 Hex/EtOAc); MS(ESI) 310.0.

1-(3-Methoxyphenyl)-cyclohexylamine. To a 50 mL round-bottom flask was added the tert-butyl sulfinimine (1.16 g, 3.75 mmol), MeOH (20 mL) and HCl/dioxane (5 mL) and allowed to stir at room temperature for 30 min. Disappearance of starting material was monitored by HPLC. The reaction was concentrated yielding a solid which was dried, rinsed with Et₂O (2×), and dried again in vacuo. MS(ESI) 189.0.

### EXAMPLE 69

### 5-BROMO-2-IMIDAZOL-1-YL-BENZONITRILE

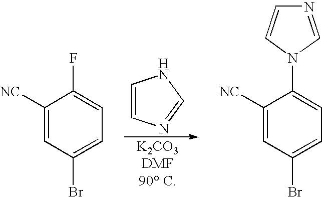

5-Bromo-2-imidazole-1-yl-benzynitrile. To a stirred solution 5-bromo-2-fluorobenzonitrile (50.0 g, 250 mmol) in DMF (300 mL) was added K₂CO₃ (69 g, 500 mmol), and then imidazole (20.0 g, 300 mmol). The reaction mixture was heated to 90° C. and stirred overnight. The reaction mixture was diluted with water and extracted with EtOAc (2×). The organic layer was washed with water and brine, dried with sodium sulfate, filtered, and concentrated. Hexane was added to the resulting solid and allowed to stir for 5 min then filtered off leaving a white solid.

### EXAMPLE 70

### 4-(3-TERT-BUTYL-PHENYL)-TETRAHYDRO-PYRAN-4-YLAMINE

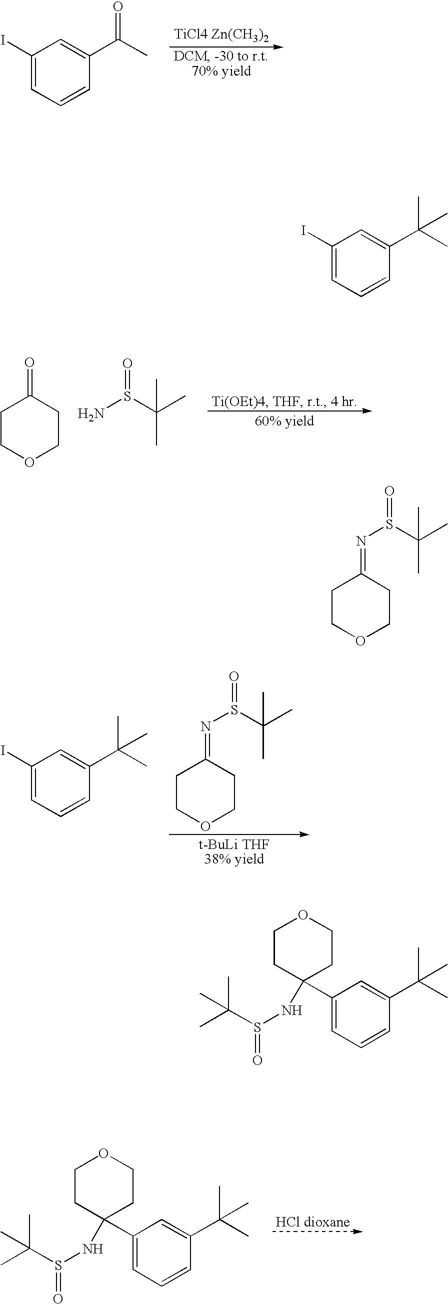

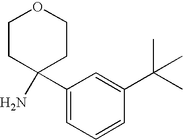

1-tert-Butyl-3-iodo-benzene. To a cooled (−40° C.) stirred solution of TiCl₄ (11 mL of a 1.0M sol in DCM, 11 mmol) in 5 mL of DCM was added dimethyl zinc (5.5 mL of a 2 N sol. in toluene, 11 mmol). After stirring for 10 min iodoacetophenone (1.23 g, 5.0 mmol) was added. After 2 h the reaction was warmed to 0° C. and stirred for an additional 1 h. The reaction was poured onto ice and extracted with ether. The organic phase was washed with water and sat NaHCO₃. The organic phase was dried over magnisium sulfate, filtered, and dried under reduced pressure. The material was distilled using a kugelrohr (80° C. at 0.1 mm) to obtain 1.0 g (76% yield) of a clear oil.

2-Methyl-propane-2-sulfinic acid (tetrahydro-pyran-4-ylidene)-amide. To a stirred solution of tetrahydro-pyran-4-one (1.2 g, 12 mmol) in 20 mL THF at room temperature under nitrogen was added titanium (IV) ethoxide (4.8 g, 21 mmol) followed by 2-methyl-propane-2-sulfinic acid amide (1.29 g, 10 mmol). The reaction was stirred at room temperature for 3 h. The reaction was quenched by pouring it into 20 mL of saturated sodium bicarbonate stirring rapidly. The formed precipitate was filtered off through GF/F filter paper and rinsed with EtOAc. The aqueous layer was washed once with EtOAc. The combined organics dried (magnesium sulfate), filtered and concentrated to a yellow oil. The material was purified using a biotage 40M cartridge eluting with hexane:ethyl acetate (60:40) to yield 1.25 g (62% yield) of a clear oil.

### EXAMPLE 71

### ALTERNATIVE PREPARATION OF 5-(2,2-DIMETHYL-PROPYL)-2-IMIDAZOL-1-YL-BENZYLAMINE

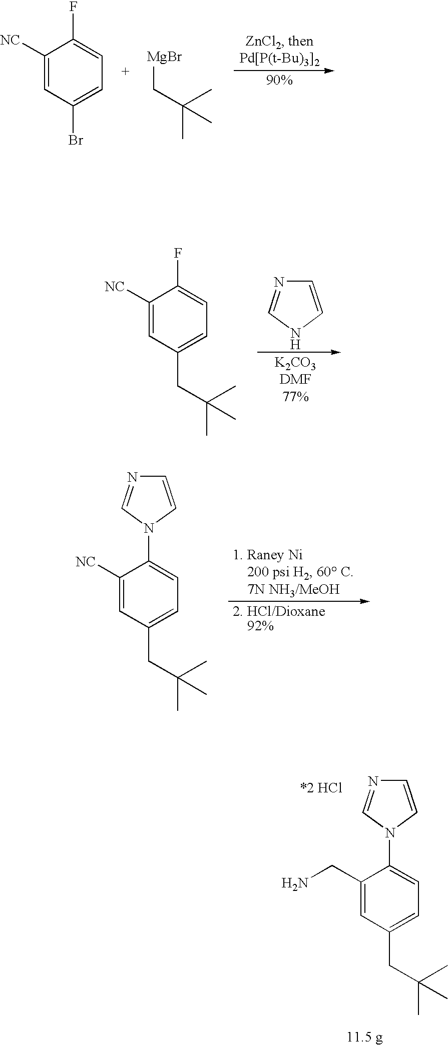

Incorporation of the neopentyl group was performed using a Negishi coupling with the neopentyl zinc species generated from the commercially available neopentylmagnesium chloride. The in situ generated neopentyl zinc reagent underwent cross-coupling reaction with the aryl bromide using the Fu catalyst at room temperature. Displacement of the aryl fluoride with imidazole occurred in DMF with heating. Reduction of the nitrile was carried out with Raney Ni. During the reduction, a significant amount of dimer was seen when Boc anhydride was used instead of ammonia. The reaction was found to proceed to completion at 200 psi of hydrogen at 60° C. Reduction of the temperature to either 20° C. or 40° C. or reducing the pressure of H₂(g) significantly reduced the rate of the reduction. The product was an oil, but treating with hydrogen chloride in dioxane gave the salt as a free flowing solid.

### Step 1: Preparation of 5-neopentyl-2-fluoro-benzonitrile

To a solution of zinc chloride (50 mL, 1.0M in diethyl ether, 50 mmol) was added neopentylmagnesium chloride (50 mL, 1.0 M in THF, 50 mmol) dropwise at 0° C. During the addition, the generated magnesium salts formed a white precipitate. The reaction was removed from the ice bath and allowed to stir for 1 h then 1-bromo-2-fluorobenzonitrile (5 g, 25 mmol) was added followed by bis(tri-tert-butylphosphine)palladium (0.127 g, 0.25 mmol, 1%). The reaction began to reflux and was placed back into the ice bath. After 1 h, the reaction was diluted with 200 mL of diethyl ether and washed with 1 N HCl (2×100 mL), brine (100 mL), dried over magnesium sulfate and concentrated yielding an oily solid (4.3 g, 22 mmol, 90%).

### Step 2: Preparation of 5-neopentyl-2-imidazol-1-yl-benzonitrile

A solution of 5-neopentyl-2-fluoro-benzonitrile (4.3 g, 22.5 mmol), imidazole (1.68 g, 24.73 mmol) and potassium carbonate (6.25 g, 44.97 mmol) were stirred in DMF (50 mL) at 90° C. The reaction was stopped after 4 h and worked up, but LCMS and ¹H NMR show starting material remaining. The crude product was resubmitted to reaction conditions and stirred overnight. The reaction was diluted with ethyl acetate (100 mL) and washed with water (2×75 mL) and brine (75 mL). The organic layer was dried over magnesium sulfate and concentrated yielding a white solid (4.16 g, 17.4 mmol, 77%); MH+240.2.

### Step 3: Preparation of 5-neopentyl-2-fluoro-benzylamine

To a solution of 5-neopentyl-2-imidazol-1-yl-benzonitrile (10.00 g, 41.79 mmol) in ammonia in methanol solution (7 N, 350 mL) was added a slurry of Raney nickel (10 mL). The reaction was sealed in a parr bomb and placed under H₂ (200 psi) then heated to 60° C. As the pressure dropped, H₂ was added to adjust the pressure to 200 psi. After 8 h, the vessel was cooled, the hydrogen was removed and the reaction was placed under N₂(g). The reaction was filtered, washed with methanol and concentrated. The resulting oil was dried for 48 h. The oil was dissolved in 50 mL of diethyl ether and 4N HCl in dioxane (32 mL) was added which caused a precipitate to form. This precipitate was collected by filtration, washed with diethyl ether (100 mL) and methylene chloride (100 mL). Drying under high vacuum gave a white solid (12.1 g, 38.3 mmol, 92%); MH+244.2.

6-(2,2-Dimethyl-propyl)-2,3-dihydro-1H-quinolin-4-one. To a stirred solution of the β-lactam (20.1 g, 92.5 mmol) in 300 mL dichloroethane at 0° C. under nitrogen was added triflic acid (27.76 g, 185 mmol) dropwise by syringe. The reaction was allowed to warm to room temperature. HPLC showed that the reaction had proceeded to completion after about 4 h. The reaction was poured into 1 L of rapidly stirred 1:1 CH₂Cl₂:ice cold saturated NaHCO₃. After stirring for a few minutes the organics were isolated and the aqueous solution extracted 1×200 mL with CH₂Cl₂. The combined organics were dried (magnisium sulfate), filtered and concentrated to a yellow oil (20.1 g, 100%); LC retention time=3.87 min.

### EXAMPLE 72

### PREPARATION OF 4-AMINO-4-(3-TERT-BUTYLPHENYL)-PIPERIDINE-1-CARBOXYLIC ACID BENZYL ESTER

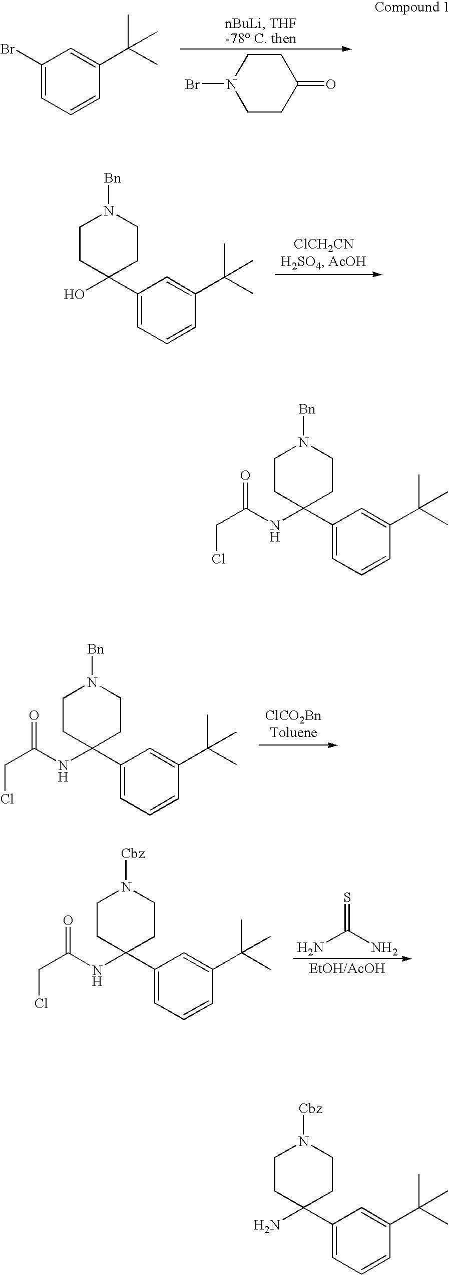

1-Benzyl-4-(3-tert-butylphenyl)-piperidin-4-ol. A solution of bromo-tert-butylbenzene (4.62 g, 21.68 mmol) in THF (50 mL) was cooled to −78° C. then n-BuLi (2.5M, 9.1 mL) was added dropwise. The reaction was stirred for 30 min then a solution of 1-benzyl-piperidin-4-one (3.69 g, 19.5 mmol) in THF (10 mL) was added dropwise. After stirring for 30 min at −78° C., the reaction was warmed to 0° C. then quenched with water (50 mL). The reaction was diluted with ethyl acetate (100 mL); the organic was separated, washed with brine (50 mL), dried over magnesium sulfate and concentrated yielding an oil (6.94 g, 21.5 mmol), which was used in the next step without further purification; LC rt=2.98 min; MS(ESI) 306.2.

N- [1-Benzyl-4-(3-tert-butylphenyl)-piperidin-4-yl]-2-chloroacetamide. To 1-benzyl-4- (3-tert-butylphenyl)-piperidin-4-ol (6.94 g, 21.45 mmol) and chloroacetonitrile (3.24 g, 75.50 mmol) was added acetic acid (3.5 mL) then sulfuric acid (3.5 mL) and the reaction stirred at room temperature overnight. The reaction was diluted with ethyl acetate (100 mL), washed with ammonium chloride (100 mL), water (50 mL), brine (50 mL), then dried over magnesium sulfate and concentrated. Silica gel chromatography eluting with 100% ethyl acetate gave an oil (2.75 g, 6.89 mmol); MS(ESI) 399.3.

4-(3-tert-Butylphenyl)-4-(2-chloroacetylamino)-piperidine-1-carboxylic acid benzyl ester. To a solution of N-[1-benzyl-4-(3-tert-butylphenyl)-piperidin-4-yl]-2-chloroacetamide (2.65 g, 6.664 mmol) in toluene (20 mL) was added benzyl chloroformate (1.90 mL, 7.00 mmol) and the reaction was heated to 80° C. The reaction was concentrated, placed onto silica gel and eluted with hexane/ethyl acetate (2:1). Isolated an oil (2.82 g, 6.37 mmol); MS(ESI) 442.9.

4-Amino-4-(3-tert-butylphenyl)-piperidine-1-carboxylic acid benzyl ester. A solution of 4-(3-tert-butylphenyl)-4-(2-chloroacetylamino)-piperidine-1-carboxylic acid benzyl ester (2.82 g, 6.37 mmol) and thiourea (0.53 g, 7.00 mmol) in 10 mL of ethanol and 2 mL of acetic acid was heated to 80° C. overnight. The reaction was cooled, diluted with ethyl acetate (50 mL), washed with 1 N NaOH (50 mL), brine (50 mL), dried over magnesium sulfate and concentrated. Silica gel chromatography eluting with 5% MeOH/DCM gave some product and some mixed fractions. The mixed fractions were chromatographed over silica gel eluting with 3% MeOH/DCM and again gave some product and some mixed fractions. Finally, the mixed fractions were chromatographed over silica gel eluting with 8% MeOH/EtOAc and all impurities were removed. The batches of pure product were combined and dried yielding a colorless oil (1.60 g, 4.44 mmol, 69%); LC retention time=3.15 min; MS(ESI) 350.0;

### EXAMPLE 73

### PREPARATION OF 4-(3-TERT-BUTYLPHENYL)-TETRAHYDRO-2H-PYRAN-4-AMINE

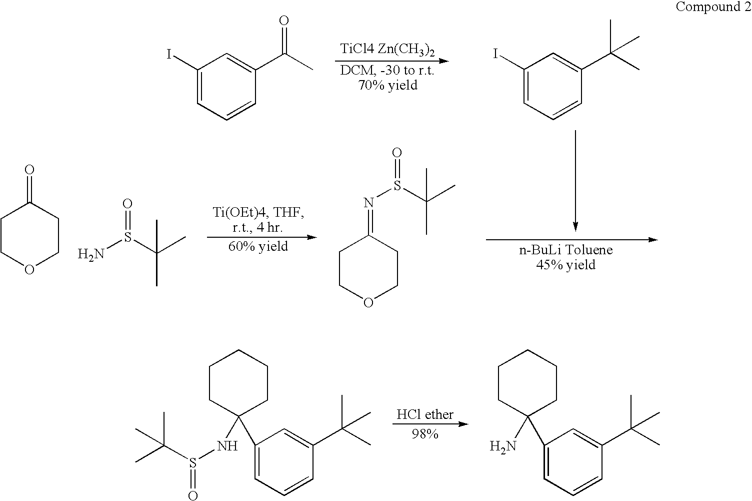

1-tert-Butyl-3-iodo-benzene. To a cooled (−40° C.) stirred solution of TiCl₄ (11 mL of a 1.0 M sol in DCM, 11 mmol) in 5 mL of DCM was added dimethyl zinc (5.5 mL of a 2 N sol. in toluene, 11 mmol). After stirring for 10 min Iodoacetophenone (1.23 g, 5.0 mmol) was added. After 2 h the reaction was warmed to 0° C. and stirred for an addtional 1 h. The reaction was poured onto ice and extracted with ether. The organic phase was washed with water and sat NaHCO₃. The organic phase was dried over magnisium sulfate, filtered, and dried under reduced pressure. The material was distilled using a kugelrohr (80° C. at 0.1 mm) to obtain 1.0 g (76% yield) of a clear oil; ¹H NMR (300 MHz, CDCl₃); δ 7.71 (t, J =2.0 Hz, 1H), 7.51 (dt, J=7.7, 1.3 Hz, 1H), 7.35 (app d, J=7.7 Hz, 1H), 7.03 (t, J=7.9 Hz, 1H), 1.29 (s, 9H).

2-Methyl-propane-2-sulfinic acid (tetrahydro-pyran-4-ylidene)-amide. To a stirred solution of tetrahydro-pyran-4-one (1.2 g, 12 mmol) in 20 mL THF at room temperature under nitrogen was added titanium (IV) ethoxide (4.8 g, 21 mmol) followed by 2-Methyl-propane-2-sulfinic acid amide (1.29 g, 10 mmol). The reaction was stirred at room temperature for 3 h. The reaction was quenched by pouring it into 20 mL of saturated sodium bicarb, while stirring rapidly. The formed precipitate was filtered off by filtration through GF/F filter paper and rinse with EtOAc. The aqueous layer was washed once with etoac. The combined organics dried (magnisium sulfate), filtered and concentrated to a yellow oil. The material was purified using a biotage 40 M cartridge eluting with hexanes:ethyl acetate (60:40) to yield 1.25 g (62% yield) of a clear oil.

2-Methyl-propane-2-sulfinic acid [4-(3-tert-butyl-phenyl)-tetrahydro-pyran-4-yl]-amide. Iodo t-butyl benzene (14 g, 54.6 mmol) was taken up in 50 mL of Toulene under N₂ and cooled to 0° C. Butyl lithium (34 mL, 1.6M sol. in hexanes) was added dropwise over 15 min. The reaction was stirred at 0° C. for 3 h. In a separate flask the imine (5.28 g, 26 mmoles) was taken up in 30 mL of Toluene and cooled to −78° C. Trimethyl aluminum (14.3 mL, 2.0 mmol sol. in toluene) was added dropwise over 10 min. The imine solution was stirred for 10 min and then cannulated into the phenyl lithium over 30 min. The reaction was allowed to warm to room temperature and stirred for 4 h. The reaction was quenched with sodium sulfate decahydrate until the bubbling stopped. Magnisium sulfate was added to the reaction and stirred for 30 min. The reaction was filtered, rinsed with etoac and concentrated down onto silica gel. The material was purified using a biotage 75S cartridge eluting with ethyl acetate to yield 4.0 g (45% yield) of desired product.

4-(3-tert-Butyl-phenyl)-tetrahydro-pyran-4-ylamine. To a stirred solution of 2-methyl-propane-2-sulfinic acid [4-(3-tert-butyl-phenyl)-tetrahydro-pyran-4-yl]-amide (3.7 g, 11.0 mmol) in ether (10 mL) was added HCl (33 mL, 1 M sol. in ether). The reaction was stirred for 30 min and then concentrated under reduced pressure; LC retention time=2.07 min; MS(ESI) 233.7.

### EXAMPLE 74

### PREPARATION OF 8-(3-TERT-BUTYL-PHENYL)-1,4-DIOXA-SPIRO[4.5]DEC-8-YLAMINE

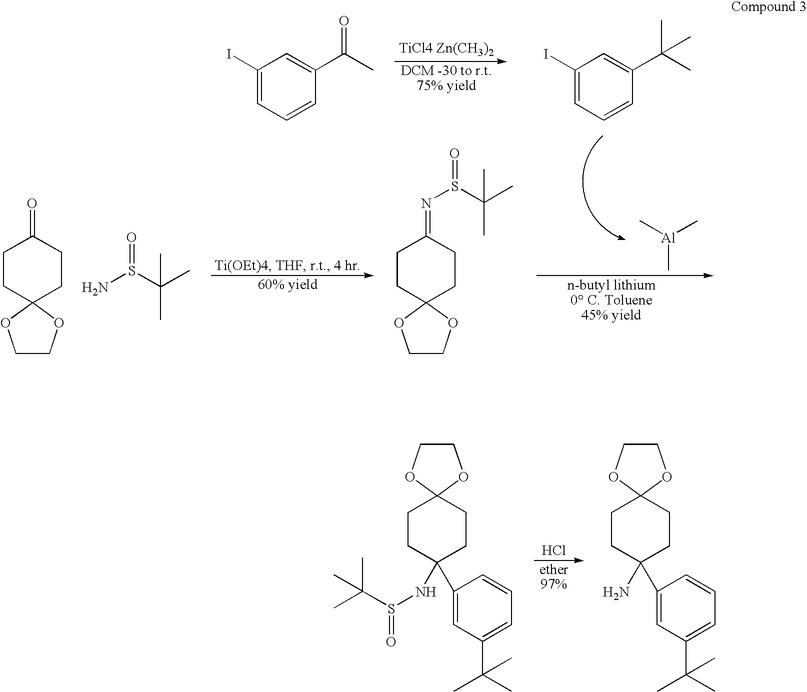

2-Methyl-propane-2-sulfinic acid (1,4-dioxa-spiro[4.5]dec-8-ylidene)-amide. To a stirred solution of 1,4-cyclohexanedione monoethylene acetal (10 g, 64.05 mmol) in 130 mL THF at room temperature under nitrogen was added titanium (IV) ethoxide (29.22 g, 128.1 mmol) followed by 2-methyl-propane-2-sulfinic acid amide (7.39 g, 61.0 mmol). The reaction was stirred at room temperature for 3 h. The reaction was quenched by pouring into 120 mL of saturated sodium bicarb. stirring rapidly. The 10 formed precipitate was filtered off by filtration through GF/F filter paper and rinsed with EtOAc. The aqueous layer was washed once with etoac. The combined organics were dried (magnisium sulfate), filtered and concentrated to a yellow oil. The material was purified using a biotage 75M eluting with hexanes:ethyl acetate (60:40) to yield 9 g (57%) of a white solid; ¹H NMR (300 MHz, CDCl₃); δ 4.00 (s, 4H), 3.16-3.05 (m, 1H), 2.95-2.83 (m, 1H), 2.62 (t, J=6.9 Hz, 2H), 1.99-1.81 (m, 4H), 1.23 (s, 9H).

2-Methyl-propane-2-sulfinic acid [8-(3-tert-butyl-phenyl)-1,4-dioxa-spiro[4.5]dec-8-yl]-amide. Iodo t-butyl benzene (38.4 g, 148 mmol) was taken up in 200 mL of Toulene under N₂ and cooled to 0° C. Butyl lithium (86.8 mL, 1.7 M sol. in hexanes) was added dropwise over 15 min. The reaction was stirred at 0° C. for 3 h. In a separate flask the imine (18.67 g, 72 mmol) was taken up in 100 mL of Toluene and cooled to −78° C. Trimethyl aluminum (37.8 mL, 2M sol. in toluene) was added dropwise over 10 min. The imine solution was stirred for 10 min and then cannulated into the phenyl lithium over 30 min. The reaction was allowed to warm to room temperature and stirred for 4 h. The reaction was quenced with sodium sulfate decahydrate until the bubbling stopped. Magnisium sulfate was added to the reaction and stirred for 30 min. The reaction was filtered, rinsed with etoac and concentrated down onto silica gel. The material was purified using a biotage 75 M cartridge eluting with ethyl acetate to yield 10 g (35% yield) of desired product; ¹H NMR (300 MHz, CDCl₃); δ 7.55 (s, 1H), 7.39-7.26 (m, 3H), 4.04-3.88 (m, 4H), 3.49 (s, 1H), 2.59-2.45 (m, 1H), 2.39-2.17 (m, 3H), 1.98-1.85 (m, 1H), 1.85-1.58 (m, 3H), 1.32 (s, 9H), 1.14 (s, 9H).

8-(3-tert-Butyl-phenyl)-1,4-dioxa-spiro[4.5]dec-8-ylamine. To a stirred solution of 2-Methyl-propane-2-sulfinic acid [8-(3-tert-butyl-phenyl)-1,4-dioxa-spiro[4.5]dec-8-yl]-amide (10 g, 25.4 mmol) in ether (30 mL) was added HCl (76.2 mL, 1M sol. in ether). The reaction was stirred for 30 min and then concentrated under reduced pressure to yield 7.35 g of the HCl salt; ¹H NMR (300 MHz, DMSO-d₆) δ 8.45 (s, 3H), 7.58 (s, 1H), 7.48-7.35 (m, 3H), 3.94-3.88 (m, 2H), 3.87-3.81 (m, 2H), 2.47-2.36 (m, 2H), 2.20-2.08 (m, 2H), 1.89-1.77 (m, 2H), 1.49-1.37 (m, 2H), 1.32 (s, 9H); LC retention time=2.77 min; MS(ESI) 289.8.

### EXAMPLE 75

### PREPARATION OF 8-(3-BROMO-PHENYL)-1,4-DIOXA-SPIRO[4.5]DEC-8-YLAMINE

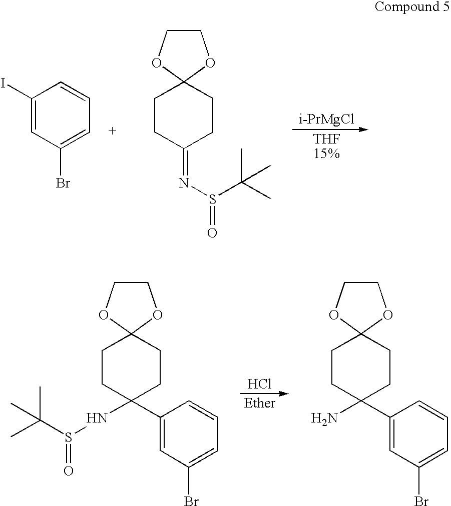

2-Methyl-propane-2-sulfinic acid [8-(3-bromo-phenyl)-1,4-dioxa-spiro[4.5]dec-8-yl]-amide. In a flask containing 3-bromo-1-iodobenzene (1.63 mL, 13 mmol) in THF (30 mL) at −40° C. was added isopropyl magnesium chloride (6.4 mL, 13 mmol) dropwise. After stirring for 2 h, the reaction was then cooled to −78° C. and the ketal imine (2.2 g, 8.5 mmol) was added. The reaction was allowed to warm to room temperature and stirred overnight. The reaction was quenched with HCl, diluted with EtOAc, then washed with brine, magnisium sulfate and dried in vacuo yielding a reddish oil. The material was purified using silica gel chromatography (1:1 Hex/EtOAc) giving a 15% yield; MS (ESI) 296.9; ¹H NMR (300 MHz, CDCl₃); δ 7.64 (s, 1H), 7.47 (app d, J=8.0 Hz, 1H), 7.41 (app d, J=8.0 Hz, 1H), 7.24 (t, J=8.0 Hz, 1H), 4.01-3.91 (m, 4H). 3.52 (s, 1H), 2.49-2.38 (m, 1H), 2.29-2.18 (m, 3H), 1.98-1.86 (m, 1H), 1.84-1.58 (m, 3H), 1.17 (s, 9H).

8-(3-bromo-phenyl)-1,4-dioxa-spiro[4.5]dec-8-ylamine. To the starting material was added HCl/ether and allowed to stir at room temperature for 2 h. The reaction was then sonicated for 10 min, filtered, washed with ether and dried yielding a white solid; LC retention time=2.48 min; ¹H NMR (300 MHz, DMSO-d₆) δ 7.82 (t, J=2.0 Hz, 1H), 7.65 (app t, J=7.2 Hz, 2H), 7.47 (t, J=8.0 Hz, 1H), 4.01-3.96 (m, 2H), 3.96-3.91 (m, 2H), 2.64-2.54 (m, 2H), 2.20 (ddd, J=14.1, 11.1, 3.9 Hz, 2H), 1.89-1.79 (m, 2H), 1.56 (dt, J=13.8, 3.5 Hz, 2H).

### EXAMPLE 76

### PREPARATION OF 1-[3-(1,1-DIMETHYL-2-TRISOPROPYLSILANYLOXYETHYL)-PHENYL]-CYCLOHEXYLAMINE

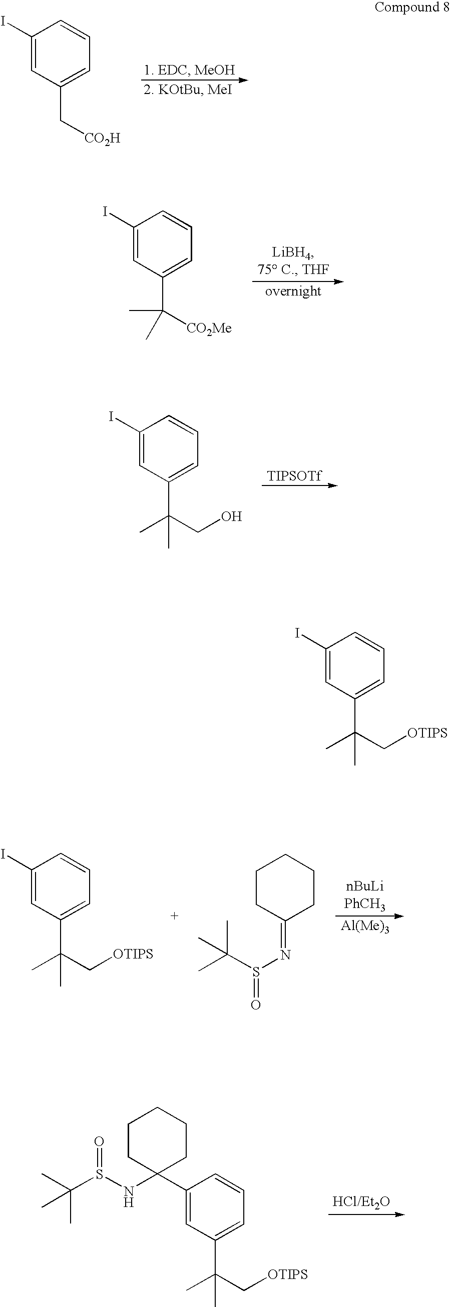

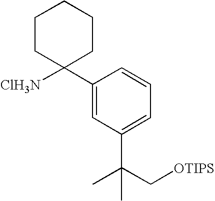

(3-Iodophenyl)-acetic acid methyl ester. To a 125 mL round-bottom flask in a 0° C. ice bath was added 3-iodobenoic acid (5.0 g, 23 mmol), EDCl (5.0 g, 26 mmol), DCM (50 mL) and allowed to stir for 10 min. To the stirred solution was added DMAP (0.3 mg, 2.3 mmol) and methanol (1.1 mL) and the reaction allowed to stir overnight. Disappearance of SM was monitored by HPLC. Reaction mixture was diluted with DCM, washed with 1 N HCl, dried with magnisium sulfate, and concentrated in vacuo. Required column chromatography (10:1 Hex/EtOAc) to isolate product (4.56 g, 87%); LC retention time=3.77 min.

2-(3-Iodophenyl)-2-methyl-propionic acid methyl ester. To a 500 mL round-bottom flask under an atmosphere of nitrogen was added (3-iodophenyl)-acetic acid methyl ester (4.56 g, 16.5 mmol), iodomethane (2.3 mL, 36 mmol), dry THF (165 mL) and allowed to stir and cool to −78° C. To the cooled reaction was added potassium t-butoxide (36 mL, 36 mmol) and allowed to stir for 2 h. The reaction mixture was diluted with 1M HCl, extracted with EtOAc, dried with magnisium sulfate, and concentrated under vacuo. Required column chromatography (5% EtOAc/Hex) to isolate product (4.74 g, 96%); LC retention time=4.25 min.

2-(3-Iodophenyl)-2-methyl-proan-1-ol. To a 500 mL round-bottom flask was added 2-(3-iodophenyl)-2-methyl-propionic acid methyl ester (4.7 g, 27 mmol), THF (275 mL), lithium borohydride (20 mL, 1.5 eq) and allowed to stir at 80° C. for 2 days. The reaction was worked up with HCl, EtOAc, brine, dried with magnisium sulfate, and concentrated in vacuo. The reaction required flash chromatography (10:1 Hex/EtOAc) to separate product from remaining starting material (4.19, 97%); LC retention time=3.66 min.

[2-(3-Iodophenyl)-2-methyl-propoxy]-triisopropylsilane. To 50 mL round-bottom flask at 0° C. was added DIEA (1.9 mL, 11 mmol), TIPS triflate (2.2 mL, 8 mmol) and allowed to stir for 10 min. To the stirred solution was added 2-(3-iodophenyl)-2-methyl-proan-1-ol (2.0 g, 7 mmol) in DCM (15 mL). The reaction was complete after 30 min. The reaction mixture was diluted with water, extracted with EtOAc, dried with magnisium sulfate, filtered and dried in vacuo. The product was then purified through a flash column using 100% Hexane (3.05, 98%).

2-Methylpropane-2-sulfinic acid{1-[3-(1,1-dimethyl-2-triisopropylsilanyloxyethyl)-phenyl]-cyclohexyl}-amide. To a 50 round-bottom flask at 0° C. was added [2-(3-iodophenyl)-2-methyl-propoxy]-triisopropylsilane (3.05 g, 7 mmol), n-butyl lithium (4.4 mL, 7 mmol), dry toluene (10 mL) and allowed to warm to room temperature for 1 h. After 1 h in a separate flask at −78° C. was added the aryl imine (1.3 g, 6 mmol), toluene (5 mL), trimethyl aluminum (3.35 mL, 6.7 mmol) and stirred for 30 min. While this was stirring for 30 min the transmetalated species was cooled to −78° C. and allowed to stir for 30 min. The Imine mixture was then added to the phenyl lithium via cannulation. The reaction was allowed to warm to room temperature and stirred for 2 h. The reaction was quenched with sodium sulfate decahydrate until the bubbling stopped. Magnisium sulfate was added to the reaction and stirred for 30 min. The reaction was filtered, rinsed with EtOAc and concentrated. The product was then purified (5:1 Hex/EtOAc) to get a clear viscous oil (1.2 g, 33.5%); MS (ESI) 530.3.

1-[3-(1,1-Dimethyl-2-trisopropylsilanyloxyethyl)-phenyl]-cyclohexylamine. To 2-methylpropane-2-sulfinic acid{1-[3-(1,1-dimethyl-2-triisopropylsilanyloxyethyl)-phenyl]-cyclohexyl}-amide (1.2 g, 2.4 mmol) was added HCl/ether and allowed to stir at room temperature for 2 h. The reaction was filtered, washed with ether and dried yielding a white solid (1.03 g); LC retention time=4.18 min; MS (ESI) 403.8.

### EXAMPLE 77

### PREPARATION OF HETEROARYL ANALOGS

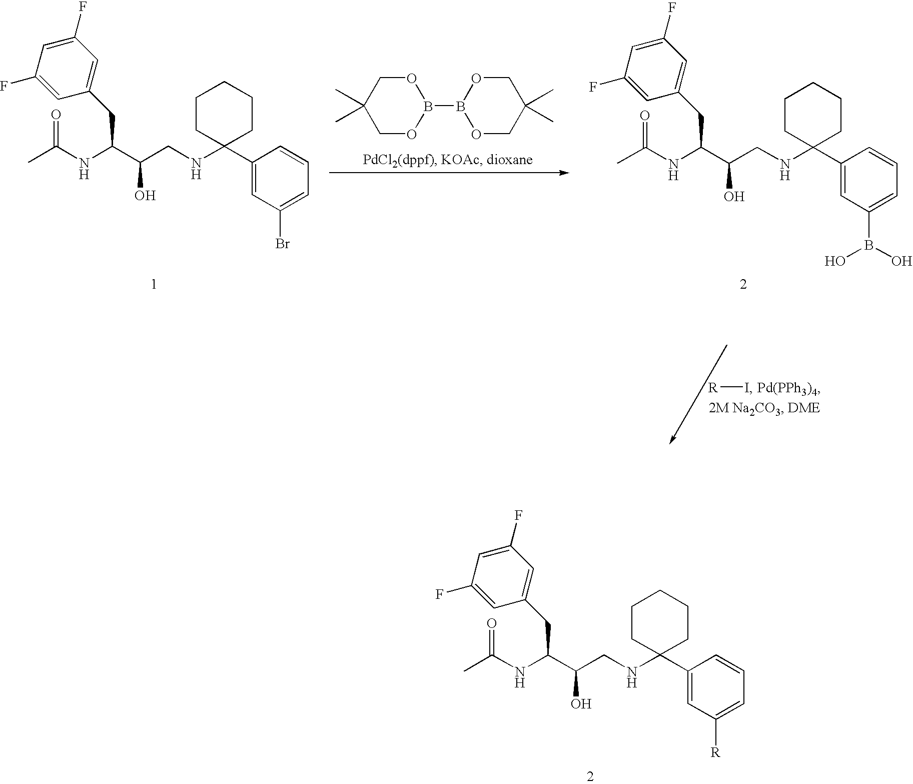

To a solution of 50 mg (˜0.1 mmol) of N-[3-[1-(3-Bromo-phenyl)-cyclohexylamino]-1-(3,5-difluoro-benzyl)-2-hydroxy-propyl]-acetamide 1 in 1 mL of dioxane is added a solution of neopentyl glycolato diboron (0.12 mmol), potassium acetate (0.4 mmol), and 1,1′-Bis(diphenylphosphino)ferrocene-palladium (II) dichloride dichloromethane complex (0.003 mmol) in 1 mL of dioxane, under nitrogen. This reaction is stirred at 90° C. for 15 h. The reaction mixture is then concentrated and 3-{1-[3-Acetylamino-4-(3,5-difluoro-phenyl)-2-hydroxy-butylamino]-cyclohexyl}-phenylboronic acid 2 is isolated via preparative HPLC.

Compound 2 (33 mg, 0.07 mmol), tetrakis(triphenylphosphine) palladium(0) (0.007 mmol), and 0.15 mL of aqueous 2M Na₂CO₃ is dissolved in 1 mL DME in a 4-mL reaction vial. Under nitrogen, a solution of the halide (0.1 mmol) in 1 mL DME is added to the reaction mixture. The reaction is then stirred at 95° C. for 15 h. The crude reaction mixture is then filtered of any solid particulates. The reaction mixture is then concentrated and the product 3 is isolated via preparative HPLC. LC/MS analysis is conducted utilizing method [1].

### EXAMPLE 78

### PREPARATION OF HETEROARYL ANALOGS

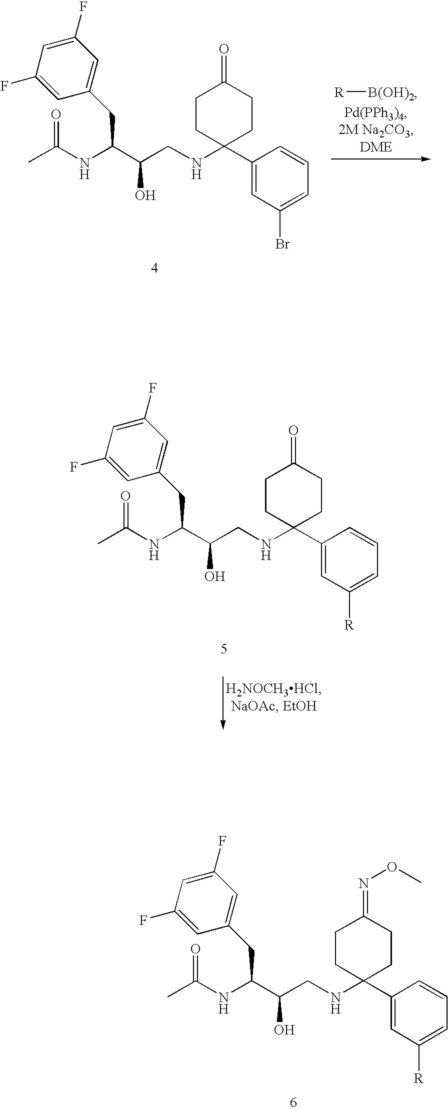

25 mg (0.04 mmol) of the N-[3-[1-(3-Bromo-phenyl)-4-oxo-cyclohexylamino)-1-(3,5-difluoro-benzyl)-2-hydroxy-propyl]-acetamide 4 is dissolved in 1 mL DME and placed in a 4-mL reaction vial. Under nitrogen, a solution of the boronic acid (0.06 mmol), tetrakis(triphenylphosphine) palladium(0) (0.006 mmol), and 0.125 mL of aqueous 2 M Na₂CO₃ dissolved in 1 mL DME is added to the reaction mixture. The reaction is then stirred at 95° C. for 15 h. The reaction mixture is then concentrated.

The product 5 (0.048 mmol) from the previous reaction is then dissolved in 1 mL ethanol and placed in a 4-mL reaction vial. Methoxylamine hydrochloride (0.23 mmol) and sodium acetate (0.13 mmol) are added in the vial. The reaction is then stirred for 2.5 h at room temperature. The reaction mixture is then concentrated and the product 6 is isolated via preparative HPLC. LC/MS analysis is conducted utilizing method [1].

### EXAMPLE 79

### PREPARATION OF N-TERMINAL HETEROARYL ANALOGS

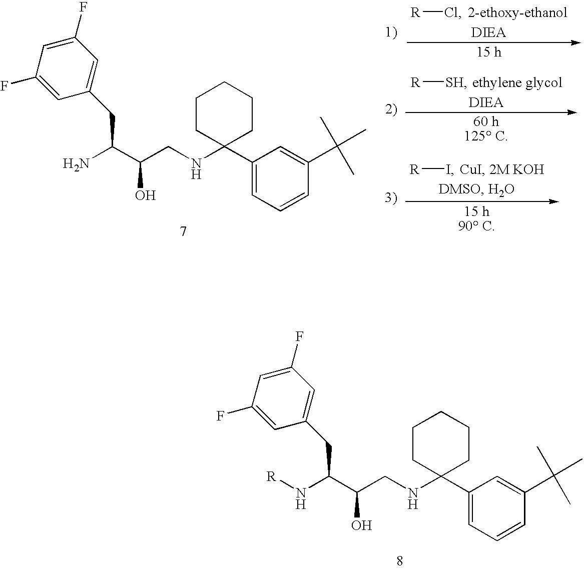

From heteroaryl chlorides. 44 mg (0.1 mmol) of 3-Amino-1-[1-(3-tert-butyl-phenyl)-cyclohexylamino]-4-(3,5-difluoro-phenyl)-butan-2-ol (7), dissolved in 1 mL of 2-ethoxy-ethanol, is placed into a 4-mL reaction vial. A solution of the heteroaryl chloride (0.1 mmol) and diisopropylethylamine (0.4 mmol) in 1 mL of 2-ethoxy-ethanol is added into the reaction vial. The reaction is then stirred for 15 h at room temperature. The reaction mixture is then concentrated and the product 8 is isolated via preparative HPLC.

From heteroaryl thiols. 44 mg (0.1 mmol) of 7, dissolved in 0.5 mL of ethylene glycol, is placed into a 4-mL reaction vial. A solution of the heteroaryl thiol (0.2 mmol) and diisopropylethylamine (0.4 mmol) in 0.5 mL ethylene glycol is added into the reaction vial. The reaction is stirred for 60 h at 125° C. The reaction mixture is then concentrated and the product 8 is isolated via preparative HPLC.

From heteroaryl iodides. 44 mg (0.1 mmol) of 7, dissolved in 0.5 mL DMSO, is placed into a 4-mL reaction vial. A solution of the heteroaryl iodide (0.15 mmol), copper iodide (0.005 mmol), and aqueous potassium hydroxide (0.5 mmol) in 1 mL DMSO is added to the reaction vial. The reaction is stirred for 15 h at 90° C. The crude reaction mixture is then filtered of any solid particulates. The reaction mixture is then concentrated and the product 8 is isolated via preparative HPLC. LC/MS analysis is conducted utilizing method [1].

### EXAMPLE 80

### PREPARATION OF N-[3-[1-(4-TERT-BUTYL-PHENYL)-CYCLOHEXYLAMINO]-1-(3,5-DIFLUORO-BENZYL)-2-HYDROXY-PROPYL]-ACETAMIDE (3)

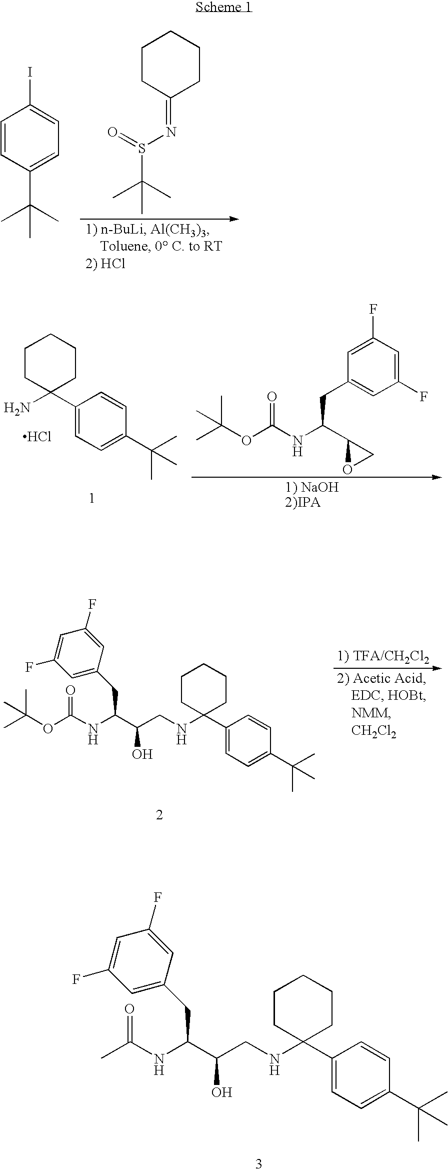

### Step 1: Preparation of 1-(4-Isopropyl-phenyl)-cyclohexylamine hydrochloride (1)

Two oven dried round-bottom flasks were cooled to room temperature by flushing with nitrogen over 30 min. One round-bottom flask was cooled to −78° C. 1-tert-Butyl-4-iodo-benzene (2.73 g, 10.43 mmol.) dissolved in 14 mL toluene was added to the round-bottom flask. The n-BuLi (2.5 M in Hexanes) (0.67 g, 10.43 mmol.) was added dropwise over 30 min. The reaction stirred at −78° C. for 1 h. The second round-bottom flask, was cooled to −78° C. 2-Methyl-propane-2-sulfinic acid cyclohexylideneamide (1.0 g, 4.97 mmol.) dissolved in 6.25 mL toluene AlMe₃ (2.0 M toluene) (0.39 g, 5.46 mmol.) was added to the round-bottom flask. The reaction in the second round-bottom flask stirred for 20 min. Then the reaction mixture in the second round-bottom flask was added by cannula to the first round-bottom flask. The reaction then stirred at −78° C. for 2 h and 0° C. for 1 h. The reaction was quenched with Na₂SO₄.6H₂O until bubbling stopped. Magnisium sulfate was added, and the reaction was stirred for 30 min. The reaction was then filtered and rinsed with EtOAc, and concentrated under reduced pressure. Crude material was purified with silica gel, eluting with 30% EtOAc in hexanes. From the column 0.26 g was recovered. The pure product was dissolved in 1.1 mL MeOH and 0.77 mL HCl (4M Dioxane). The reaction stirred at room temperature for 1 h. The reaction mixture was concentrated under reduced pressure to obtain the HCl salt of compound 1 (0.211 g of the HCl salt). MS m/z 215.1 (M-NH₂); retention time: 1.546, method: [1].

### Step 2: Preparation of [3-[1-(4-tert-Butyl-phenyl)-cyclohexylamino]-1-(3,5-difluoro-benzyl)-2-hydroxy-propyl]-carbamic acid tert-butyl ester (2)

Compound 1 was dissolved in 1 mL MeOH and added to a round-bottom flask. 2M NaOH was added until the pH was approximately 10. The reaction mixture was rinsed six times with CH₂Cl₂. The organic layer was dried with magnisium sulfate, filtered and concentrated under reduced pressure to get 0.16 g of product. The product was then dissolved in 1.0 mL isopropyl alcohol and added to a sealed tube containing [2-(3,5-Difluoro-phenyl)-1-oxiranyl-ethyl]-carbamic acid tert-butyl ester (0.22 g, 0.72 mmol). The reaction was heated to 80° C. over night. The reaction was concentrated by reduced pressure yielding 0.36 g of Compound 2. MS m/z 531.3; retention time: 2.361, method [1].

### Step 3: Preparation of N-[3-[1-(4-tert-Butyl-phenyl)-cyclohexylamino]-1-(3,5-difluoro-benzyl)-2-hydroxy-propyl]-acetamide (3)

Compound 2 was dissolved in 1 mL (1:1) trifluoroacetic acid (TFA) and CH₂Cl₂. The reaction stirred at room temperature for 2 h and concentrated under reduced pressure providing 0.298 g of product. The compound was dissolved in 4 mL CH₂Cl₂ and N-Methylmorpholine (NMM) (0.32 g, 3.12 mmol.). The reaction was stirred at 0° C. Acetic Acid (0.046 g, 0.76 mmol.) was added slowly to the reaction mixture and the mixture stirred at 0° C. for 5 min. Then 1-Hydroxylbenzotriazole hydrate (HOBt) (0.10 g, 0.76 mmol.) and 1-Ethyl-3-(3′-Dimethylaminopropyl)carbodiimide Hydrochloride (EDC.HCl) (0.15 g, 0.76 mmol.) were added sequentially. The reaction was stirred at room temperature for 2 h. CH₂Cl₂ was removed by reduced pressure and the residue dissolved in EtOAc. The organic layer was rinsed with saturated NaHCO₃ three times and once with brine. The organic layer was dried with magnisium sulfate, filtered and concentrated under reduced pressure. Compound 3 was purified by preparative HPLC (17 mg).

¹H NMR (CD₃OD); 6 7.60-7.53 (m, 4H), 6.81-6.77 (m, 3H), 3.90-3.84 (m, 1H), 3.55-3.49 (m, 1H), 3.21-3.16 (m, 1H), 2.75 (s, 1H), 2.7 (s, 1H), 2.65-2.64 (bs, 2H), 2.56-2.48 (m, 1H), 1.98-1.82 (m, 3H), 1.76 (s, 3H), 1.63 (bs, 1H), 1.41-1.38 (m, 2H), 1.35 (s, 9H), 1.30-1.25 (m, 2H). MS m/z 473.2; retention time: 1.906, method [1].

### EXAMPLE 81

### PREPARATION OF N-[3-[1-(3-TERT-BUTYL-5-IODO-PHENYL)-CYCLOHEXYLAMINO]-1-(3,5-DIFLUORO-BENZYL)-2-HYDROXY-PROPYL]-ACETAMIDE (7)

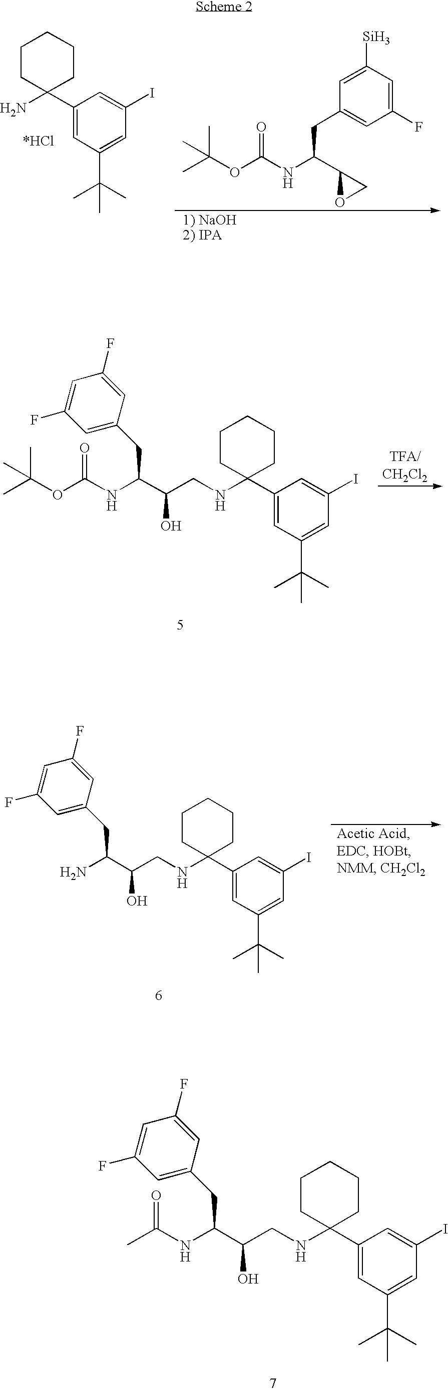

### Step 1: Preparation of [3-[1-(3-tert-Butyl-5-iodo-phenyl)-cyclohexylamino]-1-(3,5-difluoro-benzyl)-2-hydroxy-propyl]-carbamic acid tert-butyl ester (5)

2.0 g (5.08 mmol.) of 1-(3-tert-Butyl-5-iodo-phenyl)-cyclohexylamine hydrochloride and 2 mL of MeOH were added to a round-bottom flask. 2M NaOH was used to elevate the pH to approximately 10. The reaction mixture was rinsed six times with CH₂Cl₂, and the combined organic layers were concentrated under reduced pressure. The residue (1.61 g, 4.51 mmol.) and [2-(3,5-Difluoro-phenyl)-1-oxiranyl-ethyl]-carbamic acid tert-butyl ester (1.35 g, 4.51 mmol.) were added to the sealed tube with 4 mL of isopropyl alcohol. The reaction was stirred at 80° C. over night. The reaction mixture was concentrated under reduced pressure yielding 1.55 g of Compound (5). MS m/z 657.1; retention time: 2.368, method [1].

### Step 2: Preparation of 3-Amino-1-[1-(3-tert-butyl-5-iodo-phenyl)-cyclohexylamino]-4-(3,5-difluoro-phenyl)-butan-2-ol (6)

Compound 5 (1.55 g, 2.36 mmol.) was dissolved in a (1:1) solution of TFA and CH₂Cl₂. The reaction stirred at room temperature for 2 h and concentrated under reduced pressure providing 1.27 g of Compound 6. MS m/z 557.1; retention time: 1.853, method [1].

### Step 3: Preparation of N-[3-[1-(3-tert-Butyl-5-iodo-phenyl)-cyclohexylamino]-1-(3,5-difluoro-benzyl)-2-hydroxy-propyl]-acetamide (7)

Compound 6 (1.27 g, 2.28 mmol.) was dissolved in 20 mL CH₂Cl₂ and NMM (1.03 g, 10.27 mmol). The reaction was cooled to 0° C. and stirred. Acetic Acid (0.15 g, 2.51 mmol.) was added slowly to the reaction mixture and stirred at 0° C. for 5 min. Then HOBt (0.34 g, 2.51 mmol.) and EDC.HCl (0.48 g, 2.51 mmol.) were added to the round-bottom flask. The reaction stirred at room temperature for 2 h. CH₂Cl₂ was removed under reduced pressure and the crude product was dissolved in EtOAc. The organic layer was rinsed with saturated NaHCO₃ three times and brine once. The organic layer was dried with magnisium sulfate, filtered and concentrated under reduced pressure yielding Compound 7. The final compound was purified by preparative HPLC (13.0 mg).

¹H NMR (CD₃OD); δ 7.85 (s, 1H), 7.78 (s, 1H), 7.64 (s, 1H), 6.82 (s, 2H), 6.79 (s, 1H), 3.87-3.83 (m, 1H), 3.56-3.52 (m, 1H), 3.25-3.19 (m, 1H), 2.71-2.48 (m, 4H), 2.01-1.89 (m, 2H), 1.82 (s, 3H), 1.83-1.80 (m, 2H), 1.61 (bs, 1H), 1.47-1.39 (m, 2H), 1.35-1.26 (m, 2H), 1.34 (s, 9H). MS m/z 598.2; retention time: 2.100, method [1].

### EXAMPLE 82

### N-[3-[1-(3-ACETYL-5- TERT-BUTYL-PHENYL)-CYCLOHEXYLAMINO]-1-(3,5-DIFLUORO-BENZYL)-2-HYDROXY-PROPYL]-ACETAMIDE (8)

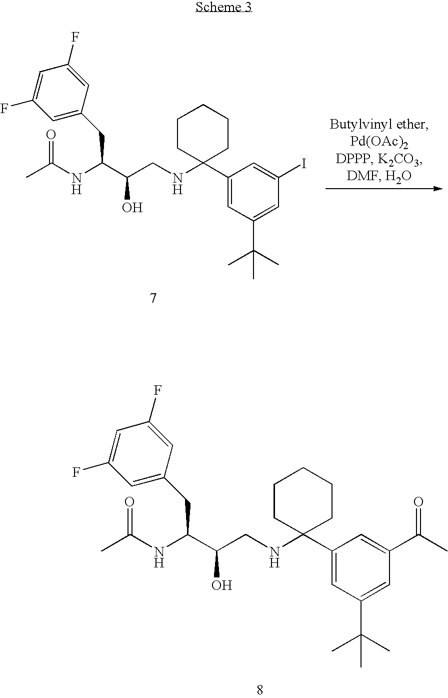

### Step 1: Procedure of N-[3-[1-(3-Acetyl-5-tert-butyl-phenyl)-cyclohexylamino]-1-(3,5-difluoro-benzyl)-2-hydroxy-propyl]-acetamide (8)

Compound 7 (0.025 g, 0.36 mmol.) was added to a sealed tube with Butyl vinyl ether (0.009 g, 0.090 mmol.), Palladium(II) Acetate (Pd(OAc)₂) (0.0002 g, 0.0011 mmol.), 1,3-Bis(diphenylphosphino)propane (DPPP) (0.001 g, 0.0024 mmol.), and Potassium Carbonate (K₂CO₃) (0.0056 g, 0.043 mmol.). 90 μL of DMF and 11 μL H₂O were added to the sealed tube. The reaction was heated to 80° C. for two days. The reaction mixture was run through a plug of diatomaceous earth and purified by preparative HPLC (25.0 mg).

¹H NMR (CD₃OD); δ 8.12-8.11 (bs, 1H), 8.04 (s, 1H), 7.92-7.91 (bs, 1H), 6.81-6.75 (m, 3H), 3.86-3.83 (m, 2H), 3.56-3.53 (m, 1H), 3.23-3.18 (m, 1H), 2.75-2.69 (m, 2H), 2.67 (s, 3H), 2.66-2.63 (m, 4H), 2.05-1.98 (m, 2H), 1.86-1.78 (m, 2H), 1.79 (s, 3H), 1.62 (bs, 1H), 1.50-1.40 (m, 2H), 1.41 (s, 9H). MS m/z 515.2; retention time: 1.842, method [1].

### EXAMPLE 83

### N-[3-[1-(3-AMINO-5- TERT-BUTYL-PHENYL)-CYCLOHEXYLAMINO]-1-(3,5-DIFLUORO-BENZYL)-2-HYDROXY-PROPYL]-ACETAMIDE (9)

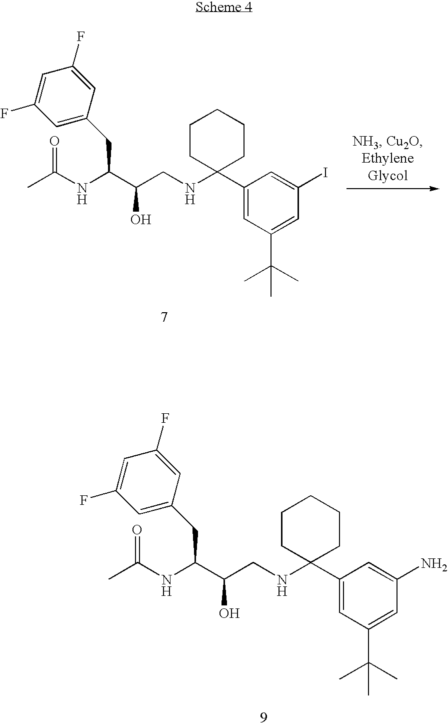

### Step 1: Procedure of N-[3-[1-(3-Amino-5-tert-butyl-phenyl)-cyclohexylamino]-1-(3,5-difluoro-benzyl)-2-hydroxy-propyl]-acetamide (9)

(Adapted from Tetrahedron Letters; 42; 2001; 3251-3254). Compound 7 (0.05 g, 0.084 mmol.) was added to 1 mL of Ethylene Glycol in a sealed tube. Copper (I) oxide (Cu₂O) was added to the sealed tube. The sealed tube was capped with a septum. The reaction mixture was then cooled to 0° C. Ammonia (NH₃) was bubbled into the reaction mixture for 30 min. The reaction mixture was then warmed to room temperature and once at room temperature the septum was quickly removed and the screw cap was added. The reaction mixture was then heated to 80° C. over night. The reaction was then run on the preparative HPLC to remove the Ethylene glycol and to purify Compound 9 (21.0 mg).

¹H NMR (CD₃OD); δ 3.32-3.30 (bs, 1H), 7.62 (s, 1H), 7.37 (s, 1H), 7.30 (s, 1H), 6.82-6.78 (m, 3H), 3.85-3.82 (m, 1H), 3.62-3.59 (m, 1H), 3.23-3.17 (m, 1H), 2.68-2.66 (m, 4H), 2.60-2.51 (m, 1H), 2.03-1.96 (m, 2H), 1.85-1.82 (m, 1H), 1.78 (s, 3H), 1.68-1.52 (m, 1H), 1.45-1.43 (m, 1H), 1.38 (s, 9H), 1.32 (m, 2H). MS m/z 488.2; retention time: 1.447, method [1].

### EXAMPLE 84

### PROCEDURE OF N-[3-[8-(3-BROMO-PHENYL)-1,4-DIOXA-SPIRO[4.5]DEC-8-YLAMINO]-1-(3,5-DIFLUORO-BENZYL)-2-HYDROXY-PROPYL]-ACETAMIDE (14)

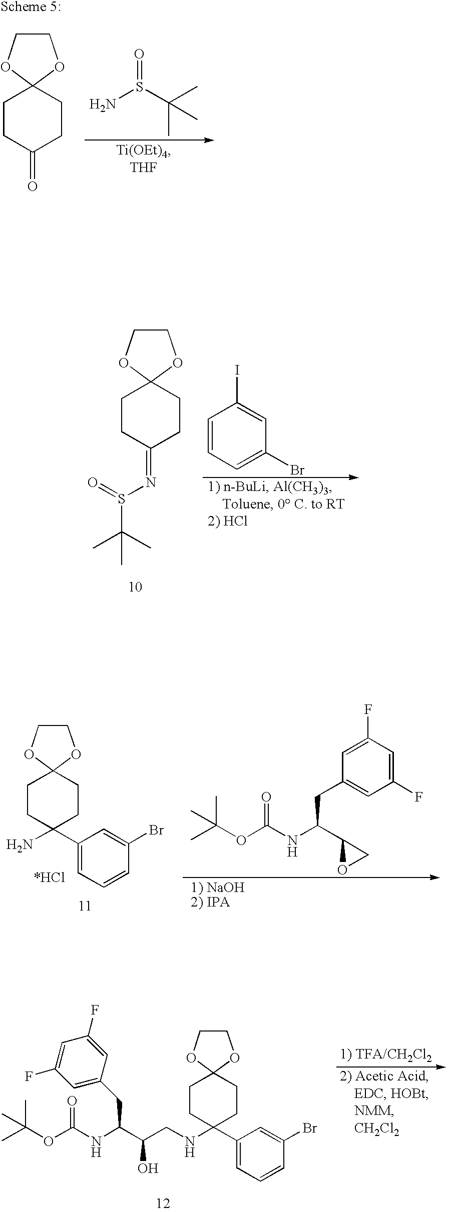

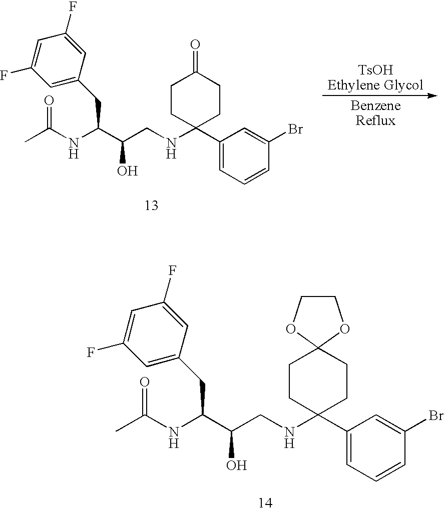

### Step 1: Procedure of 2-Methyl-propane-2-sulfinic acid (1,4-dioxa-spiro[4.5]dec-8-ylidene)-amide (10)

An oven dried round-bottom flask was cooled to room temperature by flushing with nitrogen gas for 30 min. 1,4-Dioxa-spiro[4.5]decan-8-one (1.35 g, 8.66 mmol.) (dissolved in 12 mL THF), 2-Methyl-propane-2-sulfinic acid amide (1.0 g, 8.25 mmol.) (dissolved in THF), and titanium(IV) ethoxide (3.77 g, 16.50 mmol.) were added to the round-bottom flask. The reaction was stirred for 4 h at room temperature. To the mixture was added 15 mL saturated NaHCO₃, followed by filtration and an EtOAc rinse. The organic layer was dried with magnisium sulfate, filtered and concentrated under reduced pressure yielding 0.98 g of Compound 10. MS m/z 260.1; retention time: 0.754, method [1].

### Step 2: Procedure for 8-(3-Bromo-phenyl)-1,4-dioxa-spiro[4.5]dec-8-ylamine Hydrochloride (11)

Two oven dried round-bottom flask were cooled to room temperature by flushing with nitrogen. n-Butyl Lithium (2.5 M in hexanes) (0.46 g, 7.14 mmol.) was added dropwise to a stirring solution of 1-Bromo-3-iodo-benzene (2.02 g, 7.14 mmol.) in 3.2 mL Toluene to the round-bottom flask at 0° C. The reaction stirred from 0° C. to room temperature over 2 h. A separate round-bottom flask was cooled to −78° C. and Compound (10) (0.98 g, (assuming 90% purity) 3.4 mmol.) and AlMe₃ (0.269 g, 3.74 mmol.) were added to the second round-bottom flask and stirred for 10 min. Contents in the second round-bottom flask were added by cannula to the first round-bottom flask. The combined material was at 0° C. and allowed to reach room temperature over 3 h. The reaction was then quenched with Na₂SO₄.6H₂O until bubbling stopped. Magnisium sulfate was added to the reaction mixture. The reaction mixture was then filtered and concentrated under reduced pressure. The reaction provided 1.6 g of crude material. A column on silica gel (50% EtOAc:hexanes) provided 0.29 g of pure material. The pure material was treated with 0.69 mL 4M HCl in dioxanes and stirred for 1 h at room temperature. The reaction mixture was then placed under reduced pressure. 0.23 g of Compound 11 were recovered. MS m/z 295.0 (M-NH₂); retention time: 0.979, method [1].

### Step 3: Procedure for [3-[8-(3-Bromo-phenyl)-1,4-dioxa-spiro[4.5]dec-8-ylamino]-1-(3,5-difluoro-benzyl)-2-hydroxy-propyl]-carbamic acid tert-butyl ester (12)

Same procedure was used as in EXAMPLE 3, Step 1. MS m/z 611.1; retention time: 1.919, method [1].

### Step 4: Procedure for N-[3-[1-(3-Bromo-phenyl)-4-oxo-cyclohexylamino]-1-(3,5-difluoro-benzyl)-2-hydroxy-propyl]-acetamide (13)

Same procedure as was used in EXAMPLE 81, Step 1. MS m/z 611.1; retention time: 1.919, method [1

### Step 5: Procedure for N-[3-[8-(3-Bromo-phenyl)-1,4-dioxa-spiro[4.5]dec-8-ylamino]-1-(3,5-difluoro-benzyl)-2-hydroxy-propyl]-acetamide (14)

Compound (13) (0.4 g, 0.78 mmol.), p-Toluenesulfonic acid monohydrate (TsOH) (0.16 g, 0.84 mmol.), poly(Ethylene glycol) (8.9 g, 143.4 mmol.), and 25 mL benzene were added to a round-bottom flask. The reaction was heated to 100° C. for 30 min. The benzene was removed under reduced pressure and fresh benzene was added. This was repeated no starting material was present. Once reaction was complete, it was treated with saturated NaHCO₃ and extracted CH₂Cl₂. The organic layer was washed with Brine and dried with Mg₂SO₄, filtered and concentrated under reduced pressure providing 0.4 g of Compound (14). MS m/z 553.1; retention time: 1.523, method [1].

### EXAMPLE 85

### N-{1-(3,5-DIFLUORO-BENZYL)-2-HYDROXY-3-[8-(3-PYRAZOL-1-YL-PHENYL)-1,4-DIOXA-SPIRO[4.5]DEC-8-YLAMINO]-PROPYL}-ACETAMIDE (15)

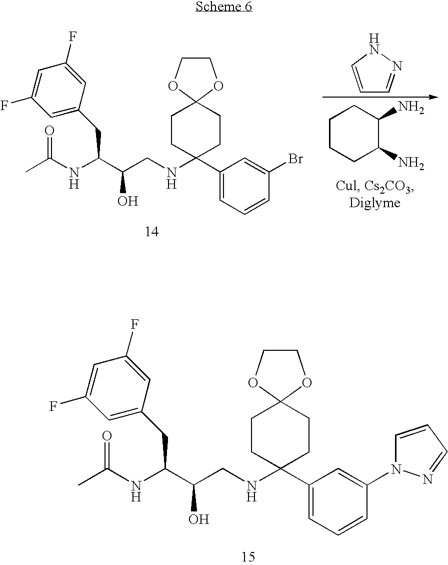

### Step 1: N-{1-(3,5-Difluoro-benzyl)-2-hydroxy-3-[8-(3-pyrazol-1-yl-phenyl)-1,4-dioxa-spiro[4.5]dec-8-ylamino]-propyl}-acetamide (15)

Compound 14 (0.4 g, 0.72 mmol.), pyrazole (0.059 g, 0.87 mmol.), and cesium carbonate (0.47 g, 1.45 mmol.) were added to a round-bottom flask. Copper(l) Iodide (0.014 g, 0.072 mmol.) and trans-1,2-diaminocyclohexanes (0.0082 g, 0.072 mmol.) were both weighed in separate vials in a nitrogen box. Diglyme was added to the trans-1,2-diaminocyclohexanes. This mixture was then transferred to the vial contain the Copper(I) Iodide which was then transferred to the round-bottom flask. The reaction mixture was then heated to 130° C. for four days. The crude material was purified by preparative HPLC (13.0 mg).

¹H NMR (CD₃OD); δ 7.87 (s, 1H), 7.72-7.65 (m, 2H), 7.52-7.46 (m, 1H), 6.82-6.79 (m, 3H), 4.00-3.87 (m, 4H), 3.57-3.54 (m, 1H), 3.23-3.17 (m, 1H), 2.83-2.65 (m, 3H), 2.58-2.53 (m, 1H), 2.27-2.19 (m, 2H), 1.87 (s, 2H), 1.80 (s, 2H), 1.80 (s, 3H), 1.51-1.29 (m, 4H). MS m/z 541.2; retention time: 1.412, method [1].

### EXAMPLE 86

### PREPARATION OF N-[3-[1-(3-TERT-BUTYL-5-METHYL-PHENYL)-CYCLOHEXYLAMINO]-1-(3,5-DIFLUORO-BENZYL)-2-HYDROXY-PROPYL]-ACETAMIDE

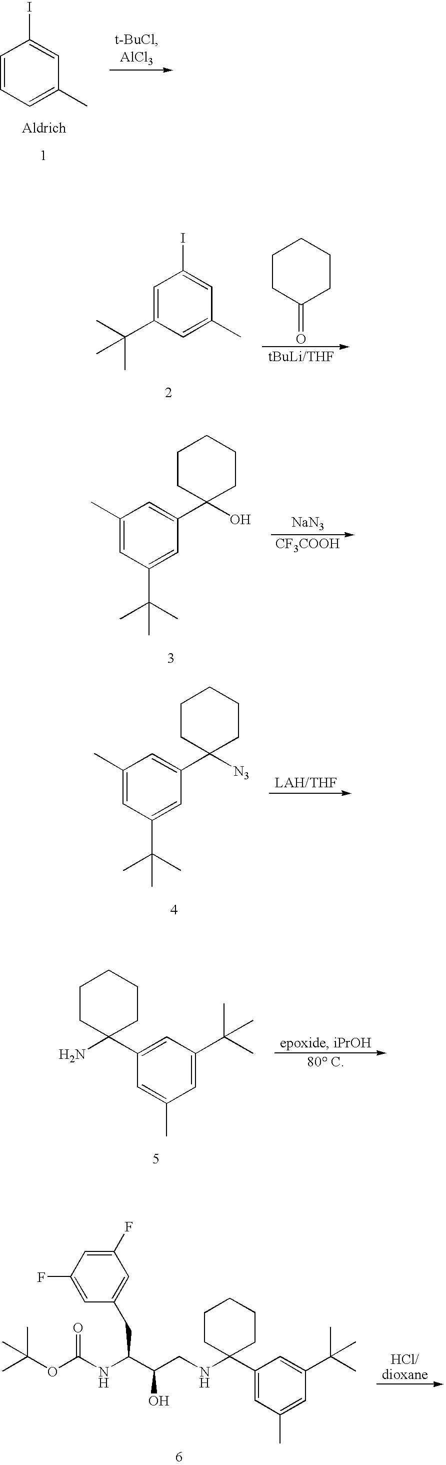

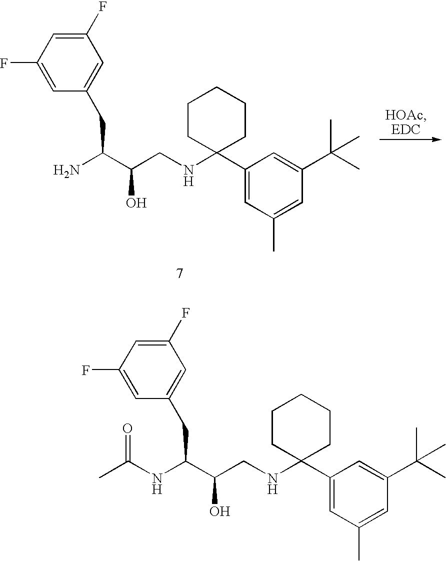

### Step 1: Preparation of 1-tert-Butyl-3-iodo-5-methyl-benzene (2)

Aluminium chloride (0.2 g) was added cautiously over 1-2 min. to a stirred, ice-cooled mixture of 3-iodotoluene (Aldrich, 6.41 mL, 50 mmol) and tert-BuCl (8 mL, 72.5 mmol). Stirring was continued for 15 min. in all. The mixture was poured into water and extracted with CH₂Cl₂. Organic layer was washed with Na₂S₂O₅, dried and concentrated. Distillation at 0.03 mmHg gave some SM (34°-38° C.) and mostly product 2 (56°-63° C.) as colorless oil. Yield 7.55 g (55%). ¹H NMR (CDCl₃); δ 7.54 (s, 1H), 7.39 (s, 1H), 7.16 (s, 1H), 2.32 (s, 3H), 1.31 (s, 9H); ¹³C NMR (CDCl₃); δ 21.2, 31.5, 34.6, 125.6, 131.6, 135.1, 138.0, 139.7.

### Step 2: Preparation of 1-(3-tert-Butyl-5-methyl-phenyl)-cyclohexanol (3)

To a 500 mL round-bottom flask, under nitrogen, added dry THF (100 mL) followed by a solution of 1-tert-Butyl-3-iodo-5-methyl-benzene (2; 7.54 g, 27.52 mmol, 1 eq) in 20 mL of dry THF. After cooling to −78° C. added tert-BuLi (32.4 mL, 1.7 M solution in pentane, 2 eq) and continued to stir at −78° C. for 15 min. Warmed to 0° C. for 30 min. Cooled down again to −78° C and added a solution of cyclohexanone ( 2.43 g, 24.8 mmol, 0.9 eq.) in dry THF (20 mL). Continued to stir at −78° C. for 45 min., then quenched with water and extracted with ether. The organic phase was washed with brine, dried over sodium sulfate and concentrated to a colorless oil (6.45 g), which was used in a next step without purification. ¹H NMR (CDCl₃); 67.22 (s, 1H), 7.15 (s, 1H), 7.11 (s, 1H), 2.36 (s, 3H), 1.90-1.65 (m, 1OH), 1.34 (s, 9H); m/z 269.2 (M+Na), 229.2 (M-OH); retention time=2.104, method [2].

### Step 3: Preparation of 1-(1-Azido-cyclohexyl)-3-tert-butyl-5-methyl-benzene (4)

The crude alcohol 3 (6.45 g, 26.2 mmol, 1 eq) was dissolved in CH₂Cl₂ (45 mL) and sodium azide (5.1 g, 78.6 mmol, 3 eq.) was added. The mixture was stirred rapidly while a solution of trifluoroacetic acid (6.1 mL, 78.6 mmol, 3 eq) in dichloromethane was added dropwise at room temperature over 40 min. After 1 hr TLC (20% EtOAc/hexane) showed no starting material. The reaction was quenched by addition of water (100 mL). The layers were separated and the aqueous layer was extracted with CH₂Cl₂ (3×50 mL). The combined organic phase was washed with 3N NH₄OH (2×40 mL) and brine, dried and concentrated. Crude yield of 4 was 5.6 g (79%). ¹H NMR (CDCl₃); δ 7.08 (s, 1H), 7.15 (s, 1H), 7.22 (s, 1H), 2.39 (s, 3H), 2.10-1.95 (m, 2H), 1.90-1.80 (m, 2H), 1.75-1.6 (m, 6H), 1.34 (s, 9H); m/z 244.2 (M-N₃); retention time=3.039, method [2].

### Step 4: Preparation of 1-(3-tert-Butyl-5-methyl-phenyl)-cyclohexylamine (5)

The crude azide 4 (5.0 g, 18.5 mmol, 1 eq) as a solution in THF (35 mL) was added dropwise over 15 min. to a slurry of lithium aluminum hydride (2.8 g, 74 mmol, 4 eq) in THF (75 mL) in an ice-bath. Upon completion of the addition, the ice-bath was removed and the mixture was allowed to warm to room temperature. It was stirred at room temperature for 1 hr, and then heated to reflux for 1 hr. The mixture was then cooled down in an ice-bath, EtOAc (6 mL), water (2.2 mL), 15% aq.NaOH (2.2 mL) and water (6.5 mL) were carefully added in succession with 5 min. stirring between each addition. The quenched mixture was then stirred for 3 hr. The aluminates were removed by filtration and washed with THF and ether. The filtrate was dried and concentrated yielding free amine. The free base was taken up in 20 mL of hexane/ether (1:1) and 4N HCl in dioxane (5 mL) was carefully added. The resulting white precipitate was collected by filtration, washed with ether and dried under vacuum to yield 3 25 g of 5 (63% as HCl salt). ¹H NMR (CDCl₃); δ 7.10 (s, 1H), 7.18 (s, 1H), 7.38 (s, 1H), 2.38 (s, 3H), 2.05-1.95 (m, 2H), 1.74-1.60 (m, 10H), 1.35 (s, 9H); ¹³C NMR (CDCl₃); 21.8, 22.5, 25.8, 31.4, 34.7, 39.4, 53.8, 119.1, 123.0, 124.0, 137.1, 149.5, 150.9; m/z 245.8 (MH+); retention time=1.820, method [1].

### Step 5: Preparation of [3-[1-(3-tert-Butyl-5-methyl-phenyl)-cyclohexylamino]-1-(3,5-difluoro-benzyl)-2-hydroxy-propyl]-carbamic acid tert-butyl ester (6)

Boc-protected amine 6 was prepared in 79% yield according to method of EXAMPLE 22, Step 1; m/z 545.0 (MH+); retention time=2.492, method [1].

### Step 6 and 7: Preparation of N-[3-[1-(3-tert-Butyl-5-methyl-phenyl)-cyclohexylamino]-1-(3,5-difluoro-benzyl)-2-hydroxy-propyl]-acetamide

Boc-protected amine 6 was treated with 4N HCl in dioxane to yield quantitatively free amine 7, which in turn was N-acetylated using standard procedure described before. Desired product was purified by HPLC and characterized by NMR: ¹H NMR (CDCl₃); δ 7.39 (s, 1H), 7.23 (s, 1H), 7.13 (s,1H), 6.74 (s, 1H), 6.71 (s, 1H), 6.65 (m, 1H), 5.95 (m, 1H), 4.12 (m, 1H), 3.65 (m, 1H), 2.75-2.40 (m, 4H), 2.38 (s, 3H), 2.1-1.97 (m, 4H), 1.87 (s, 3H), 1.78 (m, 6H), 1.32 (s, 9H); m/z 486.9 (MH+); retention time=2.165, method [1].

### EXAMPLE 87

### PREPARATION OF N-[3-[4-(3-TERT-BUTYL-PHENYL)-1-(2-HYDROXY-ETHYL)-PIPERIDIN-4-YLAMINO]-1-(3,5-DIFLUORO-BENZYL)-2-HYDROXY-PROPYL]-ACETAMIDE (1A) AND N-[3-[4-(3-TERT-BUTYL-PHENYL)-1-(2-CYANO-ETHYL)-PIPERIDIN-4-YLAMINO]-1-(3,5-DIFLUORO-BENZYL)-2-HYDROXY-PROPYL]-ACETAMIDE (1B)

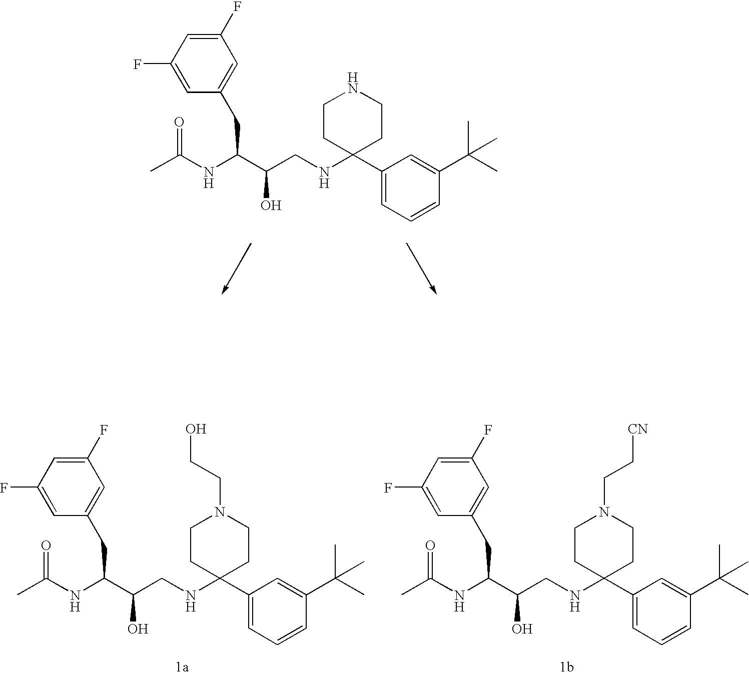

N-[3-[4-(3-tert-Butyl-phenyl)-piperidin-4-ylamino]-1-(3,5-difluoro-benzyl)-2-hydroxy-propyl]-acetamide (012 g, 0.25 mmol) was stirred and heated to 50° C. with 2-bromoethanol (0.017 mL, 0.25 mmol) and anhydrous Na₂CO₃ (0.10 g) in 6 mL of absolute EtOH. The resulting mixture was refluxed under N₂ for 2 hr. The EtOH was evaporated, and the residue was dissolved in CH₂Cl₂ and washed with brine. The dried organic phase was evaporated and purified by HPLC yielding pure alcohol 1a (0.07 g). ¹H NMR (CDCl₃); δ 7.74 (bs, 1H), 7.58-7.51 (m, 1H), 7.49-7.40 (m, 1H), 7.28 (s, 1H), 6.64-6.60 (m,3H), 6.35-6.28 (m, 1H), 4.10-3.80 (m, 4H), 3.75-3-62 (m, 2H), 3.10-2.85 (m, 6H), 2.81-2.70 (m, 2H), 2.67-2.60 (m, 2H), 2.55-2.42 (m, 2H), 1.79 (s, 3H), 1.76 (bs, 1H), 1.25 (s, 9H); m/z 518.3 (MH+); ret. time 1.309, method [1].

To a solution of piperidine N-[3-[4-(3-tert-Butyl-phenyl)-piperidin-4-ylamino]-1-(3,5-difluoro-benzyl)-2-hydroxy-propyl]-acetamide (0.06 g, 0.12 mmol) and triethylamine (0.5 mL) in methanol (3 mL) was added acrylonitrile (0.2 mL) at room temperature, and the mixture was stirred for 4 hr. Solvent and the excess of acrylonitrile were evaporated and purified by HPLC to yield nitrile 1b; m/z 527.3 (MH+); ret. time 1.408, method [1].

### EXAMPLE 88

### PREPARATION OF N-[3-[1-(3-TERT-BUTYL-PHENYL)-4-METHOXYAMINO-CYCLOHEXYLAMINO]-1-(3,5-DIFLUORO-BENZYL)-2-HYDROXY-PROPYL]-ACETAMIDE

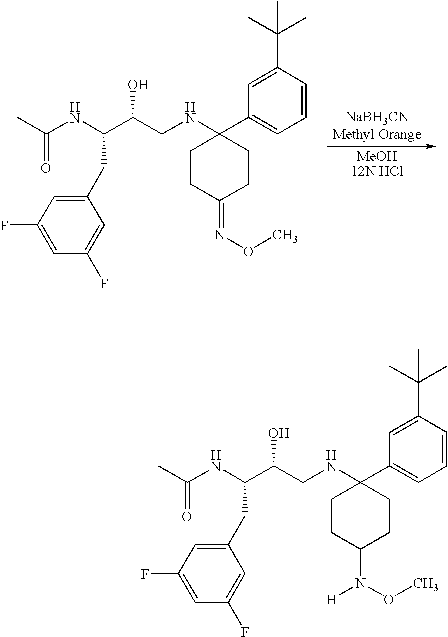

To a solution of N-[3-[1-(3-tert-butyl-phenyl)-4-methoxyimino-cyclohexylamino]-1-(3,5-difluoro-benzyl)-2-hydroxy-propyl]-acetamide (0.075 g,0.145 mmol) in methanol (0.4 mL) was added sodium cyanoborohydride (0.025 g, 0.340 mmol) and a catalytic amount of methyl orange. A solution of 12 N HCl was added dropwise until reaction mixture turned from yellow to red. The reaction was stirred at room temperature overnight under N₂ (g) inlet prior to quenching with saturated NaHCO₃ (aq.) The product was extract with CHCl₃ followed by a CHCl3/iPA (3:1) solution. The organic phases were combined, dried (sodium sulfate) and concentrated under reduced pressure. The residue was purified by HPLC chromatography using method [7] to yield trifluoroacetate salts of the separated isomers. MS (Cl): 518.3 (M+H). Retention times 1.415 and 1.559. Reference: Journal of Fluorine Chemistry, 59,1992,157-162.

### EXAMPLE 89

### PREPARATION OF N-((2S,3R)-4-(1-(3-TERT-BUTYLPHENYL)-4-(METHOXYAMINO)CYCLOHEXYLAMINO)-1-(3,5-DIFLUOROPHENYL)-3-HYDROXYBUTAN-2-YL)ACETAMIDE

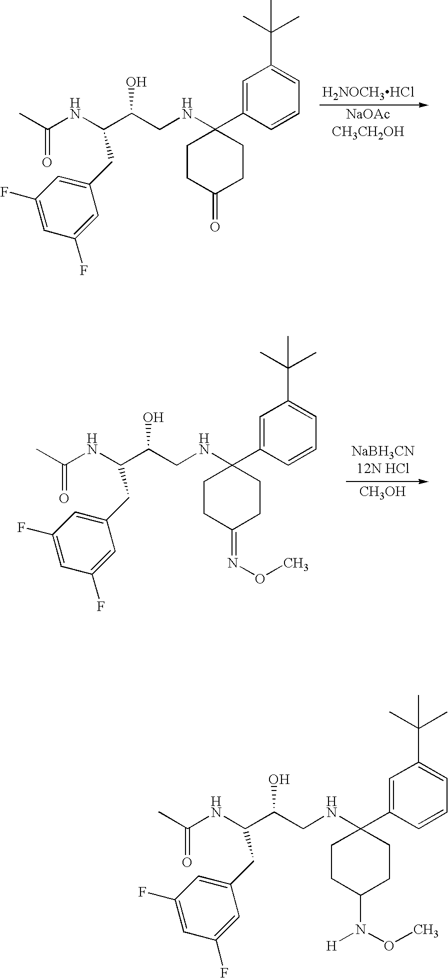

The N-((2S,3R)-4-( 1-(3-tert-butylphenyl)-4-(methoxyamino)cyclohexylamino)-1-(3,5-difluorophenyl)-3-hydroxybutan-2-yl)acetamidemethoxyl amines were acetylated under standard conditions in CH₂Cl₂. The ketone N-((2S,3R)-4-(1-(3-tert-butylphenyl)-4-oxocyclohexylamino)-1-(3,5-difluorophenyl)-3-hydroxybutan-2-yl)acetamide was converted to the methoxyl oxime and then reduced under acidic conditions with sodium cyanoborohydride. See EXAMPLE 87.

### EXAMPLE 90

### PREPARATION OF N-(4-(1-(3-TERT-BUTYLPHENYL)-4-N-HYDROXYACETAMIDO-CYCLOHEXYLAMINO)-1-(3,5-DIFLUOROPHENYL)-3-HYDROXYBUTAN-2-YL)ACETAMIDE

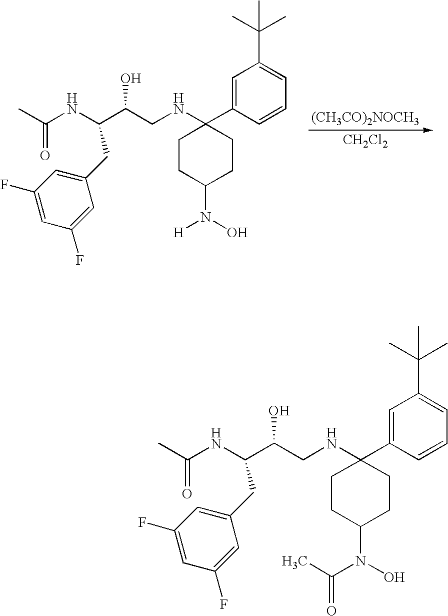

The N-((2S,3R)-4-(1-(3-tert-butylphenyl)-4-(hydroxyamino)cyclohexylamino)-1-(3,5-difluorophenyl)-3-hydroxybutan-2-yl)acetamide hydroxylamine analogs were acetylated under standard conditions with N,N-diacetyl-O-methylhydroxylamine in CH₂Cl₂ to yield N-(4-(1-(3-tert-butylphenyl)-4-N-hydroxyacetamido-cyclohexylamino)-1-(3,5-difluorophenyl)-3-hydroxybutan-2-yl)acetamide. The most polar of the diastereomers was isolated using HPLC purification.

### EXAMPLE 91

### N-[3-[1-(3-TERT-BUTYL-PHENYL)-4-HYDROXYAMINO-CYCLOHEXYLAMINO]-1-(3,5-DIFLUORO-BENZYL)-2-HYDROXY-PROPYL]-ACETAMIDE

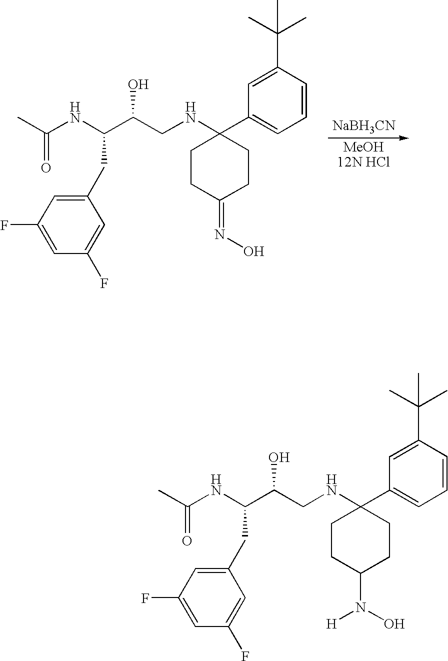

To a solution of N-[3-[1-(3-tert-butyl-phenyl)-4-hydroxyimino-cyclohexylamino]-1-(3,5-difluoro-benzyl)-2-hydroxy-propyl]-acetamide (0.138 g, 0.275 mmol) in methanol (1 mL) was added sodium cyanoborohydride (0.039 g, 0.493 mmol) and 12 N HCl dropwise until the reaction is acidic. The reaction mixture was stirred at room temperature overnight under N₂ (g) inlet prior to quenching with saturated NaHCO₃ (aq). The product was extract with CHCl₃ followed by a CHCl₃/iPA (3:1) solution. The organic phases were combined, dried (sodium sulfate) and concentrated under reduced pressure. The residue was purified by HPLC chromatography using method [7] to yield trifluoroacetate salts of the separated isomers. MS (Cl): 504.2 (M+H). Retention times: 1.361 and 1.448.

### EXAMPLE 92

### PREPARATION OF N-((2S,3R)-4-(1-(3-TERT-BUTYLPHENYL)-4-(METHOXY(METHYL)AMINO)CYCLOHEXYLAMINO)-1-(3,5-DIFLUOROPHENYL)-3-HYDROXYBUTAN-2-YL)ACETAMIDE

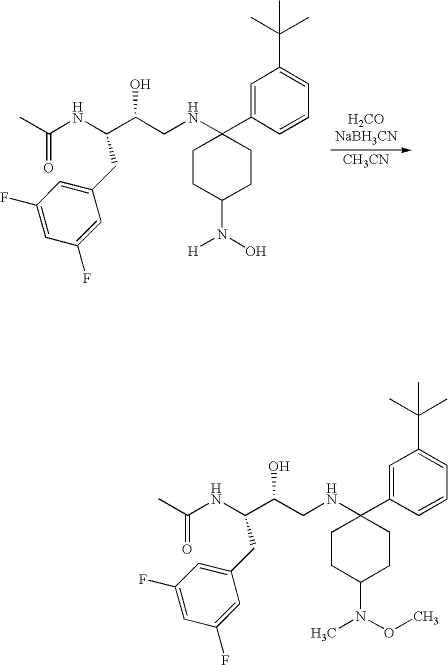

Reaction of N-((2S,3R)-4-(1-(3-tert-butylphenyl)-4-(hydroxyamino)cyclohexylamino)-1-(3,5-difluorophenyl)-3-hydroxybutan-2-yl)acetamide with aqueous formaldehyde, acetonitrile and sodium cyanoborohydride yielded N-((2S,3R)-4-(1-(3-tert-butylphenyl)-4-(methoxy(methyl)amino)cyclohexylamino)-1-(3,5-difluorophenyl)-3-hydroxybutan-2-yl)acetamide. See, e.g., Cerri, A. et al., J. Med. Chem.; 2000; 43(12); 2332-2349.

### EXAMPLE 93

### N-[3-[4-(AMINO)-1-(3-TERT-BUTYL-PHENYL)-CYCLOHEXYLAMINO]-1-(3,5-DIFLUORO-BENZYL)-2-HYDROXY-PROPYL]-ACETAMIDE

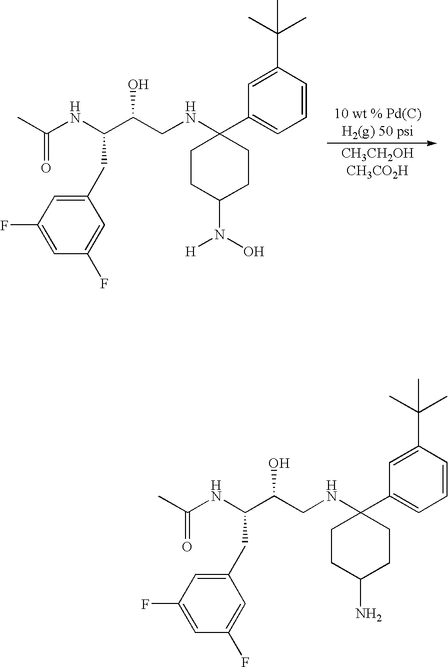

To a solution of N-[3-[1-(3-tert-butyl-phenyl)-4-hydroxyamino-cyclohexylamino]-1-(3,5-difluoro-benzyl)-2-hydroxy-propyl]-acetamide (0.050 g, 0.099 mmol) in ethanol (0.3 mL) was added a catalytic amount of 10 wt % palladium on carbon (0.72 g) and glacial acetic acid (0.05 mL). The reaction mixture was placed on the hydrogenator under 50 psi for 5 h and then filtered through celite. The reaction mixture was partitioned between saturated NaHCO₃ (aq) and EtOAc. The organic layer was separated, dried (sodium sulfate) and concentrated under reduced pressure. The residue was purified by HPLC chromatography using method [7] to yield the trifluoroacetate salt of the separated isomers. MS (Cl): 488.2 (M+H). Retention times: 1.305 and 1.413

### EXAMPLE 94

### N-[3-[1-(3-TERT-BUTYL-PHENYL)-4-METHYLAMINO-CYCLOHEXYLAMINO]-1-(3,5-DIFLUORO-BENZYL)-2-HYDROXY-PROPYL]-ACETAMIDE

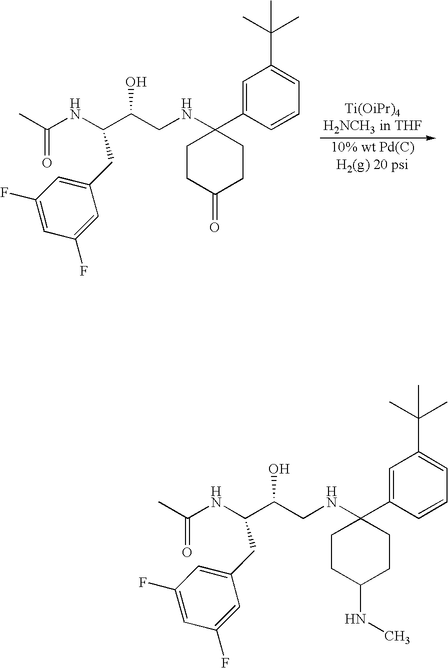

To a solution of N-[3-[1-(3-tert-butyl-phenyl)-4-oxo-cyclohexylamino]-1-(3,5-difluoro-benzyl)-2-hydroxy-propyl]-acetamide (0.155 g, 0.319 mmol) in 2.0 M solution of methylamine in THF (0.4 mL, 1.365 mmol) was added titanium (IV) isopropoxide (0.2 mL, 0.4 mmol). The reaction mixture was stirred at room temperature for 0.5 h prior to addition of a catalytic amount of 10 wt % palladium on carbon (0.16 g) and placement on the hydrogenator under 20 psi overnight. The reaction was filtered through a pad of celite and rinsed with EtOAc. The filtrate collected was washed with 1 N NaOh followed by saturated NaCl (aq). The organic layer was separated, dried (sodium sulfate) and concentrated under reduced pressure. The residue was purified by HPLC chromatography using method [7] to yield the trifluoroacetae salt of the separated isomers. MS (Cl): 502.3 (M+H). Retention times: 1.318 and 1.425. Reference: Alexakis, A. et. Al. Tet Lett. 45, 2004,1449-1451.

### EXAMPLE 95

### N-[3-[1-(3-TERT-BUTYL-PHENYL)-4-CYANO-CYCLOHEXYLAMINO]-1-(3,5-DIFLUORO-BENZYL)-2-HYDROXY-PROPYL]-ACETAMIDE

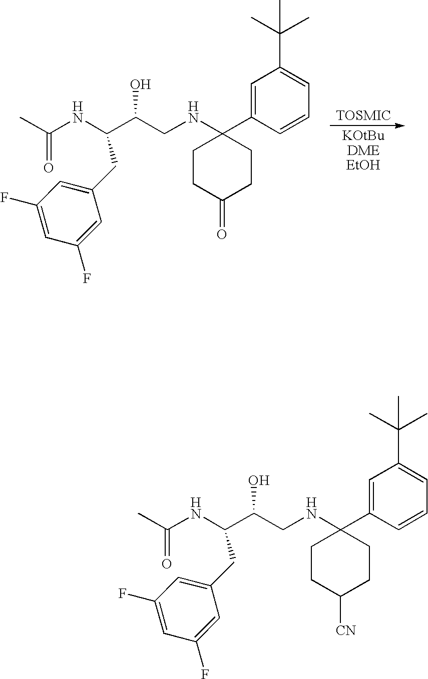

To a cooled suspension of N-[3-[1-(3-tert-butyl-phenyl)-4-oxo-cyclohexylamino]-1-(3,5-difluoro-benzyl)-2-hydroxy-propyl]-acetamide (0.098 g, 0.201 mole) in ethylene glycol dimethyl ether (0.7 mL) and absolute ethanol (0.02 mL) was added tosyl methyl isocyanide (0.066 g, 0.338 mmol) and potassium tert-butoxide (0.057 g, 0.508 mmol). The reaction mixture was warmed to room temperature while stirring for 4 h under N₂(g) inlet and then partitioned between H₂O and CHCl₃. The organic layer was separated, dried (sodium sulfate), and concentrated under reduced pressure. The residue was purified by HPLC chromatography using method [7] to yield 9.3 mg of trifluoroacetate salt. MS (Cl): 498.2 (M+H) Retention time:1.833 min. Reference: Becker, D. P & Flynn, D. L. Synthesis, 1992, 1080.

### EXAMPLE 96

### PREPARATION OF N-((2S,3R)-4-(1-(3-TERT-BUTYLPHENYL)-4-FORMAMIDOCYCLOHEXYLAMINO)-1-(3,5-DIFLUOROPHENYL)-3-HYDROXYBUTAN-2-YL)ACETAMIDE

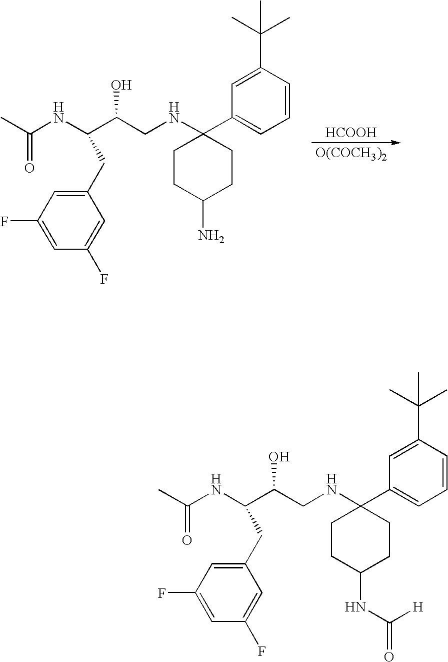

N-((2S,3R)-4-(1-(3-tert-butylphenyl)-4-formamidocyclohexylamino)-1-(3,5-difluorophenyl)-3-hydroxybutan-2-yl)acetamide was prepared from the formylation of N-((2S,3R)-4-(4-amino-1-(3-tert-butylphenyl)cyclohexylamino)-1-(3,5-difluorophenyl)-3-hydroxybutan-2-yl)acetamide with formic acid and acetic anhydride. See, e.g., Harnden, M. R., et al., J. Med. Chem.; 1990; 33(1); 187-196.

### EXAMPLE 97

### PREPARATION OF N-[3-[4-ACETYLAMINO-1-(3-TERT-BUTYL-PHENYL)-CYCLOHEXYLAMINO]-1-(3,5-DIFLUORO-BENZYL)-2-HYDROXY-PROPYL]-ACETAMIDE

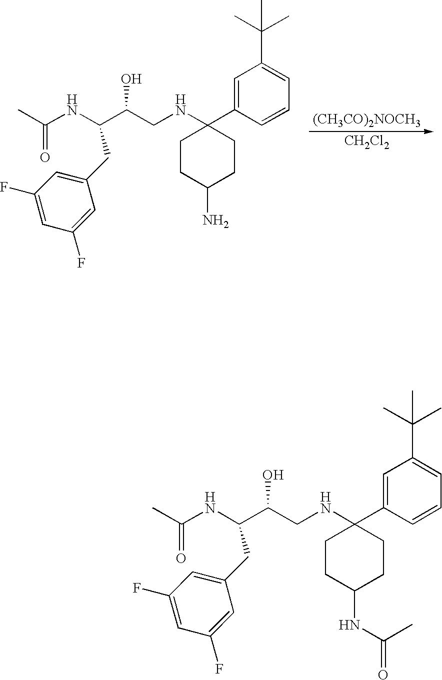

N-[3-[4-Acetylamino-1-(3-tert-butyl-phenyl)-cyclohexylamino]-1-(3,5-difluoro-benzyl)-2-hydroxy-propyl]-acetamide was synthesized via acetylation of N-((2S,3R)-4-(4-amino-1-(3-tert-butylphenyl)cyclohexylamino)-1-(3,5-difluorophenyl)-3-hydroxybutan-2-yl)acetamide with N,N-diacetyl-O-methylhydroxylamine in CH₂Cl₂.

### EXAMPLE 98

### PREPARATION OF CARBAMATE AND SULFONAMIDE ANALOGS

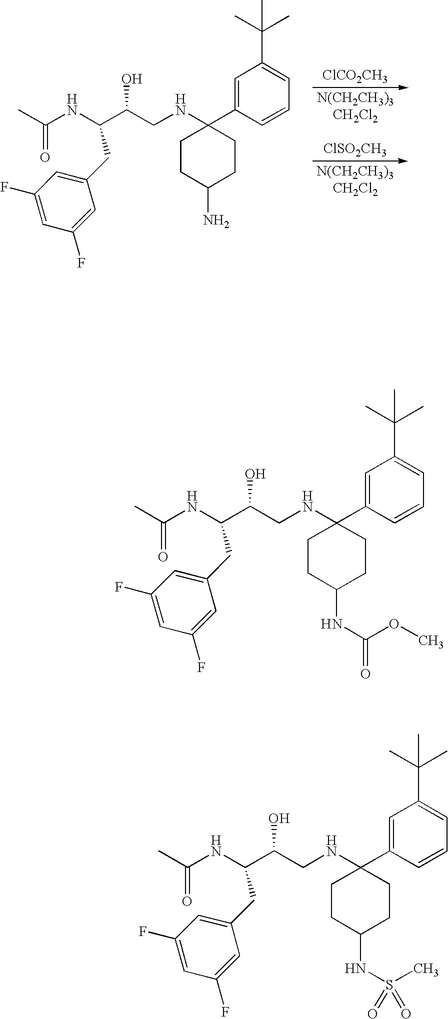

Both the carbamate (methyl 4-((2R,3S)-3-acetamido-4-(3,5-difluorophenyl)-2-hydroxybutylamino)-4-(3-tert-butylphenyl)cyclohexylcarbamate) and sulfonamide (N-((2S,3R)-4-(1-(3-tert-butylphenyl)-4-(methylsulfonamido)cyclohexylamino)-1-(3,5-difluorophenyl)-3-hydroxybutan-2-yl)acetamide) analogs were synthesized from N-((2S,3R)-4-(4-amino-1-(3-tert-butylphenyl)cyclohexylamino)-1-(3,5-difluorophenyl)-3-hydroxybutan-2-yl)acetamide with methyl chloroformate and methansulfonyl chloride, respectively, in the presence of triethylamine.

### EXAMPLE 99

### PREPARATION OF 1-(4-(3-ACETAMIDO-4-(3,5-DIFLUOROPHENYL)-2-HYDROXYBUTYLAMINO)-4-(3-TERT-BUTYLPHENYL)CYCLOHEXYL)-3-METHYLUREA

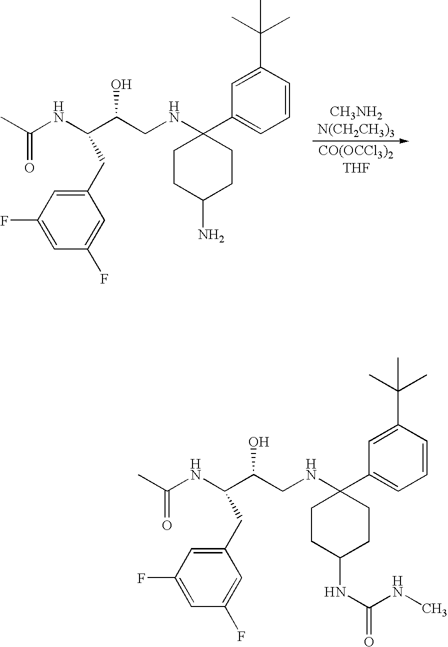

The urea compounds, (e.g., 1-(4-(3-acetamido-4-(3,5-difluorophenyl)-2-hydroxybutylamino)-4-(3-tert-butylphenyl)cyclohexyl)-3-methylurea), were synthesized from N-((2S,3R)-4-(4-amino-1-(3-tert-butylphenyl)cyclohexylamino)-1-(3,5-difluorophenyl)-3-hydroxybutan-2-yl)acetamide by treatment with triphosgene in the presence of base followed by the addition of methylamine. See, e.g., Tao, B. et al., Synthesis; 2000; 10; 1449-1453.

### EXAMPLE 100

### PREPARATION OF N-((2S,3R)-4-(1-(3-TERT-BUTYLPHENYL)-4-(2-HYDROXYETHYL)CYCLOHEXYLAMINO)-1-(3,5-DIFLUOROPHENYL)-3-HYDROXYBUTAN-2-YL)ACETAMIDE

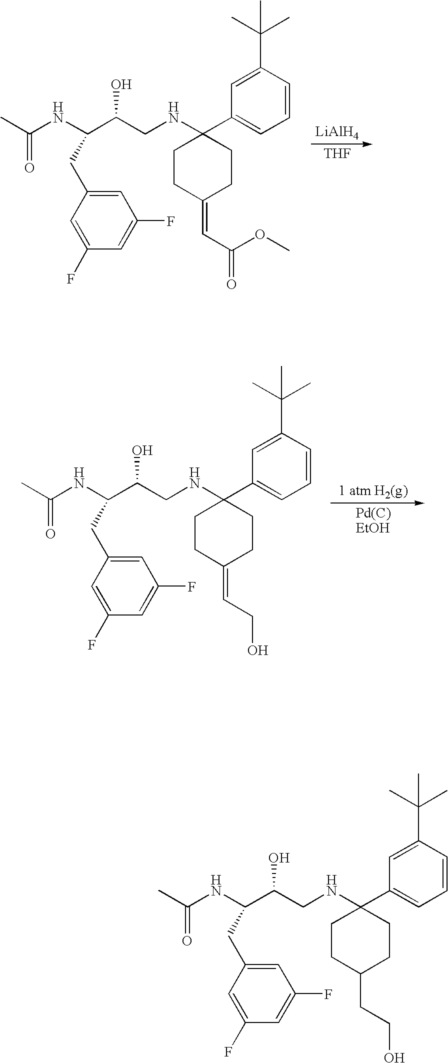

The ethyl alcohol derivative, N-((2S,3R)-4-(1-(3-tert-butylphenyl)-4-(2-hydroxyethyl)cyclohexylamino)-1-(3,5-difluorophenyl)-3-hydroxybutan-2-yl)acetamide, was prepared in two steps. First, methyl 2-(4-((2R,3S)-3-acetamido-4-(3,5-difluorophenyl)-2-hydroxybutylamino)-4-(3-tert-butylphenyl)cyclohexylidene)acetate was reduced with lithium aluminum hydride N-((2S,3R)-4-(1-(3-tert-butylphenyl)-4-(2-hydroxyethylidene)cyclohexylamino)-1-(3,5-difluorophenyl)-3-hydroxybutan-2-yl)acetamide followed by reduction of the olefin under hydrogenation conditions yielding N-((2S,3R)-4-(1-(3-tert-butylphenyl)-4-(2-hydroxyethyl)cyclohexylamino)-1-(3,5-difluorophenyl)-3-hydroxybutan-2-yl)acetamide.

### EXAMPLE 101

### CONVERSION OF TERT-BUTYL 1-(3-TERT-BUTYLPHENYL)-4-OXOCYCLOHEXYLCARBAMATE

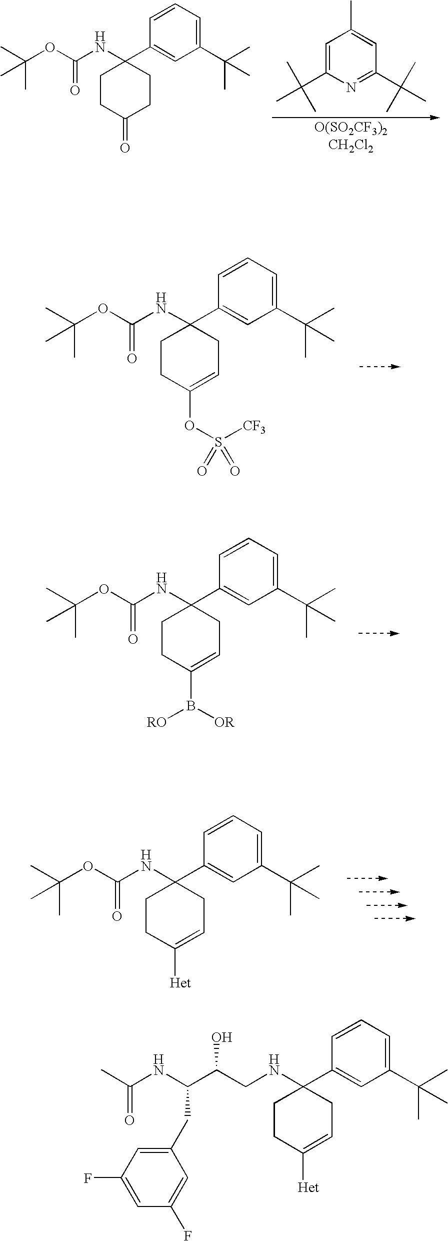

tert-butyl 1-(3-tert-butylphenyl)-4-oxocyclohexylcarbamate was converted into a vinyl triflate via treatment with 2,6-di-tert-butyl-4-methylpyridine and triflic anhydride. See, e.g., William, S. J. et al., Org. Syn.; 1983; Coll. Vol. 8; 97-103.

### EXAMPLE 102

### N-LINKED COMPOUNDS

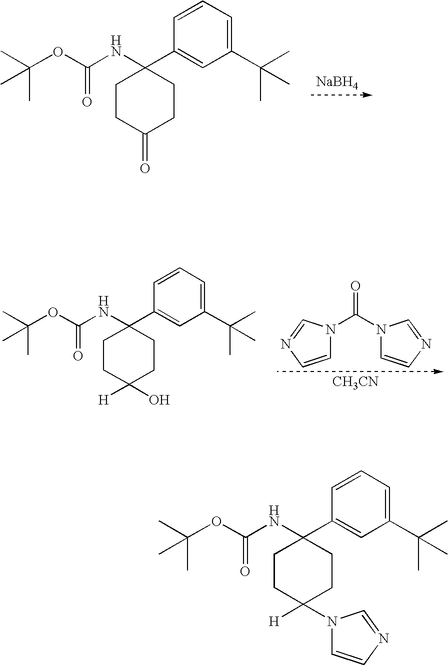

These N-linked compounds were obtained from the BOC-protected ketone amine, which can be reduced to the alcohol and then converted to the imidazole in the presence of CDI. See, e.g., Njar, V. C. O.; Synthesis; 2000; 14; 2019-2028. Similar chemistry may be utilized in order to obtain the triazole.

### EXAMPLE 103

### PREPARATION OF N-((2S,3R)-4-(1-(3-TERT-BUTYLPHENYL)-4-HYDROXY-4-(THIAZOL-2-YL)CYCLOHEXYLAMINO)-1-(3,5-DIFLUOROPHENYL)-3-HYDROXYBUTAN-2-YL)ACETAMIDE

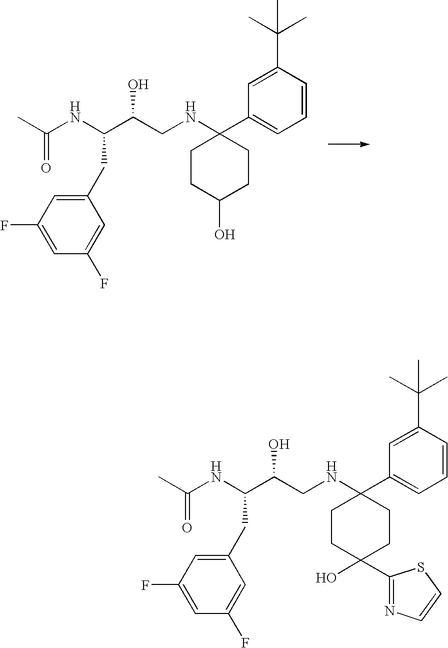

N-((2S,3R)-4-( 1-(3-tert-butylphenyl)-4-hydroxy-4-(thiazol-2-yl)cyclohexylamino)-1-(3,5-difluorophenyl)-3-hydroxybutan-2-yl)acetamide is prepared from N-((2S,3R)-4-(1-(3-tert-butylphenyl)-4-hydroxycyclohexylamino)-1-(3,5-difluorophenyl)-3-hydroxybutan-2-yl)acetamide according to methods described herein and known to those of skill in the art.

### EXAMPLE 104

### SYNTHESIS OF 4-METHYLSULFANYLCYCLOHEXANONE (5)

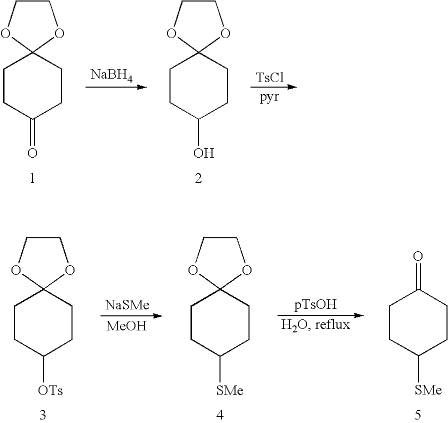

1,4-Dioxa-spiro[4.5]decan-8-ol (2) from 1,4-Dioxa-spiro[4.5]decan-8-one (1). To a solution of 1,4-dioxa-spiro[4.5]decan-8-one (Aldrich, 10.0 g, 64.0 mmol) in anhydrous methanol (250 mL) at 0° C. was added solid sodium borohydride (4.6 g, 121 mmol). The reaction mixture was then allowed to warm to room temperature over 1 h, whereupon TLC analysis indicated complete reaction. Water (60 mL) was added, and the methanol was removed under reduced pressure. The aqueous residue was partitioned between ethyl acetate (200 mL) and saturated aqueous brine (50 mL). The layers were separated, and the aqueous extracted with addition ethyl acetate (200 mL). The combined organic layers were dried (magnesium sulfate), filtered and concentrated under reduced pressure yielding the crude alcohol 2 (9.3 g, 92%): R_{f}=0.2 (CH₂Cl₂); ¹H NMR (300 MHz, CDCl₃); δ 3.95 (s, 4H), 3.85-3.75 (m, 1H), 2.00-1.75 (m, 4H), 1.75-1.50 (m, 4H).

8-Methylsulfanyl-1,4-dioxa-spiro[4.5]decane (4) from 1,4-Dioxa-spiro[4.5]decan-8-ol (2). Ref.: J. Org. Chem. 1986, 51, 2386-2388. To a solution of 1,4-dioxa-spiro[4.5]decan-8-ol (8.6 g, 54 mmol) in chloroform (54 mL) at 0° C. was added pyridine (13.2 mL, 163 mmol). To this stirring solution was added p-toluenesulfonyl chloride (20.7 g, 108 mmol) in portions. This was stirred at 0° C. for 7 h, whereupon the mixture was partitioned between diethyl ether (150 mL) and water (50 mL). The organic layer was washed with 3 N HCl (50 mL), saturated sodium bicarbonate (50 mL), and water (50 mL). The organic layer was dried (magnesium sulfate), filtered and concentrated under reduced pressure yielding crude toluene-4-sulfonic acid 1,4-dioxa-spiro[4.5]dec-8-yl ester as a crystalline solid, contaminated with p-toluenesulfonic acid: R_{f}=0.31 (CH₂Cl₂).

Crude toluene-4-sulfonic acid 1,4-dioxa-spiro[4.5]dec-8-yl ester (18 g) in ethanol (25 mL) was added to a solution of sodium thiomethoxide (12.1 g, 173 mmol) in dry methanol (75 mL). This mixture was heated to 80° C. for 4 h. The mixture was partitioned between ethyl acetate (100 mL) and water (100 mL). The aqueous layer was extracted with additional ethyl acetate (100 mL). The combined organic layers were concentrated under reduced pressure. The residue was partitioned between CH₂Cl₂ (75 mL) and saturated NaHCO₃ (100 mL). The aqueous was extracted with additional CH₂Cl₂ (50 mL). The combined organic layers were dried (sodium sulfate), filtered and concentrated under reduced pressure yielding crude 8-methylsulfanyl-1,4-dioxa-spiro[4.5]decane (6.6 g, 77% over two steps): R_{f}=0.45 (CH₂Cl₂); ¹H NMR (300 MHz, CDCl₃); δ 3.94 (s, 4H), 3.67-3.53 (m, 1H), 2.09 (s, 3H), 2.05-1.92 (m, 2H), 1.90-1.50 (m, 6H).

4-Methylsulfanyl-cyclohexanone (5) from 8-Methylsulfanyl-1,4-dioxa-spiro[4.5]decane (4). 8-Methylsulfanyl-1,4-dioxa-spiro[4.5]decane (6.6 g, 35 mmol) was combined with p-toluenesulfonic acid (6.65 g, 35 mmol) in water (75 mL), and heated to reflux for 5 h, and was subsequently allowed to stir at room temperature overnight. The aqueous reaction mixture was extracted with Et₂O (3×100 mL). The combined organic layers were washed successively with 3 N HCl (2×25 mL), saturated NaHCO₃ (2×25 mL), and water (2×25 mL). The organics were then dried (sodium sulfate), filtered and concentrated under reduced pressure. The residue was purified by flash chromatography (CH₂Cl₂ elution) yielding 4-methylsulfanyl-cyclohexanone (3.0 g, 60%): R_{f}=0.21 (3:1 CH₂Cl₂/hexanes); ¹H NMR (300 MHz, CDCl₃); δ 3.01-2.98 (m, 1H), 2.52-2.38 (m, 2H), 2.35-2.22 (m, 2H), 2.22-2.08 (m, 2H), 2.06 (s, 3H), 1.88-1.72 (m, 2H).

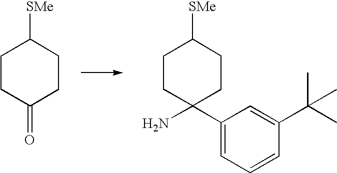

1-(3-tert-Butyl-phenyl)-4-methylsulfanyl-cyclohexylamine from 4-Methylsulfanyl-cyclohexanone. 4-Methylsulfanyl-cyclohexanone was converted into 1-(3-tert-Butyl-phenyl)-4-methylsulfanyl-cyclohexylamine in the manner described in EXAMPLE 21, except using 1-bromo-3-tert-butyl-benzene in the first step.

### EXAMPLE 105

### PREPARATION OF N-[3-[1-(3-TERT-BUTYL-PHENYL)-4-METHYLSULFANYL-CYCLOHEXYLAMINO]-1-(3,5-DIFLUORO-BENZYL)-2-HYDROXY-PROPYL]-ACETAMIDE FROM 1-(3-TERT-BUTYL-PHENYL)-4-METHYLSULFANYL-CYCLOHEXYLAMINE

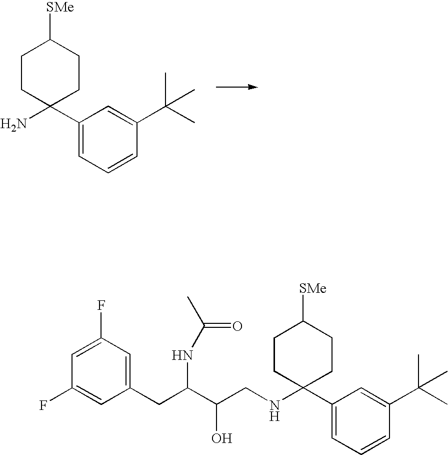

N-[3-[1-(3-tert-Butyl-phenyl)-4-hydroxymethyl-cyclohexylamino]-1-(3,5-difluoro-benzyl)-2-hydroxy-propyl]-acetamide was synthesized from 1-(3-tert-Butyl-phenyl)-4-methylsulfanyl-cyclohexylamine according to the procedure described in EXAMPLE 22 yielding (1S,2R)—N-[3-[1-(3-tert-Butyl-phenyl)-4-methylsulfanyl-cyclohexylamino]-1-(3,5-difluoro-benzyl)-2-hydroxy-propyl]-acetamide as a mixture of two isomers, which were separated by flash chromatography, and each was further purified by HPLC yielding each as trifluoroacetic acid salts:

Isomer 1: R_{f}=0.47 (EtOAc); retention time (min)=1.943, method [1]; ¹H NMR (300 MHz, CDCl₃); δ 7.64 (s, 1H), 7.50-7.28 (m, 3H), 6.75-6.55 (m, 4H), 4.10-3.90 (m, 1H), 3.82-3.70 (m, 1H), 2.97 (dd, J=15, 3 Hz, 1H), 2.83 (t, J=4.5 Hz, 1H), 2.75-2.55 (m, 2H), 2.55-2.42 (m, 2H), 2.42-2.25 (m, 3H), 2.08 (s, 3H), 1.84 (s, 3H), 1.31 (s, 9H); MS (ESI) 519.2;

Isomer 2: R_{f}=0.15 (EtOAc); retention time (min)=1.948, method [1]; ¹H NMR (300 MHz, CDCl₃); δ 7.59 (s, 1H), 7.50-7.26 (m, 3H), 6.75-6.55 (m, 3H), 6.11 (d, J=4.5 Hz, 1H), 4.12-3.98 (m, 1H), 3.73-3.60 (m, 1H), 2.97 (dd, J=12, 3 Hz, 1H), 2.90-2.60 (m, 4H), 2.50-2.00 (m, 8H), 2.05 (s, 3H), 1.83 (s, 3H), 1.32 (s, 9H); MS (ESI) 519.2.

### EXAMPLE 106

### PREPARATION OF 1-(4-BROMO-3-TERT-BUTYL-PHENYL)-CYCLOHEXYLAMINE

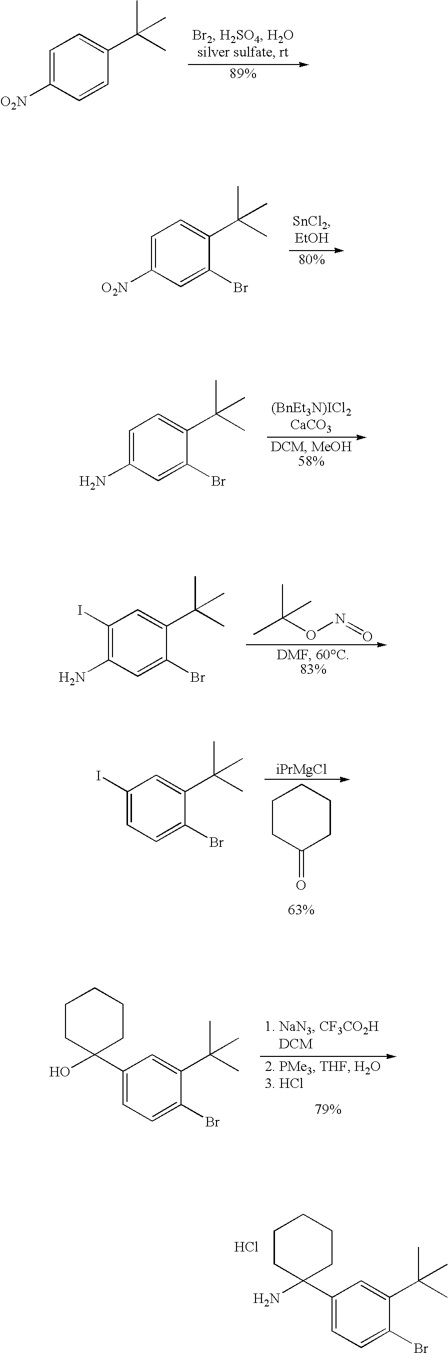

2-Bromo-1-tert-butyl-4-nitro-benzene. 1-tert-Butyl-4-nitro-benzene (21.0 g, 179.216 mmol) was dissolved in 100 mL concentrated sulfuric acid and 11 mL of water. Silver sulfate (20.1 g, 117.18 mmol) was added followed by the dropwise addition of bromine. After 3 h, the reaction was poured into dilute sodium sulfite (100 mL). The product was extracted into ethyl acetate (100 mL) washed with brine (100 mL), dried over magnesium sulfate and concentrated yielding a yellow solid 27.0 g, 89%): ¹H NMR (400 MHz, CDCl₃); δ 8.40 (d, J=2.4 Hz, 1H), 8.17 (dd, J=8.7, 2.6 Hz, 1H), 7.82 (d, J=8.7 Hz, 1H), 1.57 (s, 9H). 3-Bromo-4-tert-butyl-phenylamine. To a solution of 2-bromo-1-tert-butyl-4-nitro-benzene (5.00 g, 19.37 mmol) and tin dichloride dihydrate (21.86 g, 96.86 mmol) in ethanol (50 mL) was heated to 70° C. for 3 h. The reaction was cooled, poured into 1 N sodium hydroxide (50 mL) and the pH adjusted to 5 with 5 N sodium hydroxide. The resulting solid was removed by filtration, washed with ethyl acetate (50 mL). The aqueous layer was extracted with ethyl acetate (100 mL), washed with brine (100 mL), dried over magnesium sulfate and concentrated. Silica gel chromatography eluting first with hexane/ethyl acetate 10:1 followed by hexane/ethyl acetate 3:1 gave the product was a brownish oil (3.53 g, 80%): ¹H NMR (400 MHz, CDCl₃); δ 7.16 (d, J=8.6 Hz, 1H), 6.97 (d, J=3.0 Hz, 1H), 6.59 (dd, J=8.4, 2.8 Hz, 1H), 1.43 (s, 9H).

5-Bromo-4-tert-butyl-2-iodo-phenylamine. To a solution of 3-bromo-4-tert-butyl-phenylamine (5.00 g, 21.92 mmol) in 80 mL of DCM and 25 mL of methanol was added calcium carbonate (4.39 g, 43.83 mmol), followed by iodinating reagent (8.55 g, 21.92 mmol). The reaction was stirred for 3 h at room temperature, quenched with water 100 mL, and the product extracted into ethyl acetate (100 mL). The organic layer was washed with brine (75 mL), dried over magnesium sulfate and concentrated. Silica gel chromatography eluting with 25% ethyl acetate/hexane gave the product as an oil (4.50 g, 12.7 mmol, 58%): ¹H NMR (400 MHz, CDCl₃); δ 7.61 (s, 1H), 6.99 (t, J=0.9 Hz, 1H), 3.96 (s, 2H), 1.44 (s, 9H); MS(ESI) 356.3.

1-Bromo-2-tert-butyl-4-iodo-benzene. A solution of tert-butyl nitrite (2.56 g, 24.86 mmol) was dissolved in 30 mL of DMF and heated to 60° C. 5-Bromo-4-tert-butyl-2-iodo-phenylamine (4.40 g, 12.4 mmol) was dissolved in 10 mL of DMF and added dropwise via an addition funnel over 20 min. After the addition the reaction was stirred for 30 min at 60° C. then cooled to room temperature. The reaction was loaded onto silica gel and the product eluted with 100% hexanes as an oil (3.51 g, 10.4 mmol, 83%): ¹H NMR (400 MHz, CDCl₃); δ 7.70 (d, J=2.2 Hz, 1H), 7.34 (dd, J=6.7, 2.6 Hz, 1H), 7.28 (d, J=8.1 Hz, 1H), 1.48 (s, 9H).

1-(4-Bromo-3-tert-butyl-phenyl)-cyclohexanol. To a solution of 1-bromo-2-tert-butyl-4-iodo-benzene (3.51 g, 10.4 mmol) in THF (10 mL) at 0° C. was added isopropyl magnesium chloride (6.2 mL), after stirring for 1 h, the reaction was cooled to −78° C. and cyclohexanone (1.61 mL, 15.53 mmol) was added. The reaction was stirred for 1 h at −78° C then allowed to warm to room temperature. The reaction was quenched by the addition of 1 N HCl (50 mL) and the product extracted into ethyl acetate (75 mL). The organic layer was washed with brine (50 mL), dried over magnesium sulfate and concentrated. Silica gel chromatography eluting with a gradient of 5% ethyl acetate/hexane to 40% ethyl acetate/hexane gave the product as an oil (2.02 g, 6.49 mmol, 63%): ¹H NMR (400 MHz, CDCl₃); δ 7.62 (d, J=2.3 Hz, 1H), 7.54 (d, J=8.4 Hz, 1H), 7.14 (dd, J=8.3, 2.3 Hz, 1H), 1.84-1.60 (m, 8H), 1.52 (s, 9H), 1.37-1.24 (m, 2H).

1-(4-Bromo-3-tert-butyl-phenyl)-cyclohexylamine. To a solution of 1-(4-bromo-3-tert-butyl-phenyl)-cyclohexanol (2.02 g, 6.49 mmol) in dichloromethane (20 mL) was added sodium azide (1.27 g, 19.47 mmol) and the resulting suspension was cooled to 0° C. Trifluoroacetic acid (2.20 g, 19.47 mmol) in 10 mL of dichloromethane was added dropwise over 20 min. The reaction was removed from the ice bath and stirred for 3 h. The reaction was carefully quenched by the addition of saturated sodium bicarbonate (50 mL) and the product extracted into dichloromethane (100 mL). The organic layer was washed with brine (50 mL), dried over magnesium sulfate and concentrated yielding an oil. To this oil in tetrahydrofuran (20 mL) was added water (0.23, 12.98 mmol) followed by trimethylphosphine (0.73 mL, 7.14 mmol). The reaction was heated to 70° C. After 1 h, and additional 1 mL of water was added and stirring was continued overnight at 70° C. The reaction was cooled, concentrated, and the crude material chromatographed over silica gel eluting with a gradient from 1% methanol/ethyl acetate to 10% methanol/ethyl acetate. The product was dissolved in 5 mL of ether and 1 N HCl/ether was added (13 mL). The resulting solid was cooled by filtration, washed with hexanes and dried yielding a white solid (1.77 g, 5.10 mmol, 79% for the two steps): ¹H NMR (400 MHz, MeOD-d₄); δ 7.73 (d, J=8.2 Hz, 1H), 7.68 (d, J=2.2 Hz, 1H), 7.32 (dd, J=8.4, 2.5 Hz, 1H), 2.48 (dd, J=11.6, 3.8 Hz, 2H), 1.93 (td, J=11.8, 3.4 Hz, 2H), 1.83-1.70 (m, 2H), 1.66-1.37 (m, 4H), 1.55 (s, 9H); MS (ESI) 309.7 (⁷⁹Br).

### EXAMPLE 107

### PREPARATION OF 3-AMINO-3-(3-TERT-BUTYL-PHENYL)-PIPERIDINE-1-CARBOXYLIC ACID BENZYL ESTER

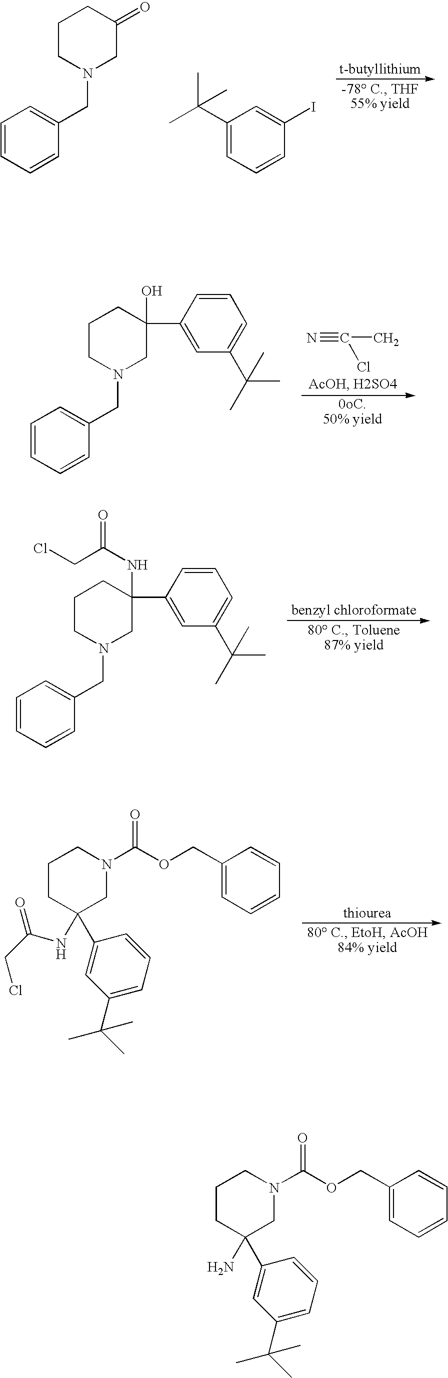

1-Benzyl-3-(3-tert-butyl-phenyl)-piperidin-3-ol. Iodo t-butyl benzene (2.46 g, 9.44 mmol) was taken up in 10 mL of THF, placed under N₂ and cooled to −78° C. T-Butyl lithium (11.06 mL, 1.7 M solution, 18.8 mmol) was added dropwise over 5 min. The reaction was allowed to stir for 1 h. The 1-benzyl-piperidin-3-one (1.5 g, 8.0 mmol) was added and the reaction was stirred for 3 h warming to room temperature. The reaction was quenched with water and extracted with ether. The ether layer was dried over magnesium sulfate, filtered and concentrated under reduced pressure. The material was purified using a biotage 40M eluting with hexanes: ethyl acetate (70:30) to yield 1.4 g (54% yield) of a clear oil: ¹H NMR (400 MHz, CDCl₃); δ 7.57 (t, J=1.3 Hz, 1H), 7.36-7.22 (m, 8H), 3.95 (s, 1H), 3.58 (s, 2H), 2.91 (d, J=10.4 Hz, 1H), 2.76 (d, J=10.8 Hz, 1H), 2.34 (d, J=10.8 Hz, 1H), 2.10-1.90 (m, 3H), 1.85-1.62 (m, 4H), 1.32 (s, 9H).

N-[1-Benzyl-3-(3-tert-butyl-phenyl)-piperidin-3-yl]-2-chloro-acetamide. To 1-benzyl-3-(3-tert-butyl-phenyl)-piperidin-3-ol (517 mg, 1.6 mmol) and chloroacetonitrile (241 mg, 3.2 mmol) was added 300 uL of AcOH. This mixture was placed under nitrogen and cooled to 0° C. Sulfuric acid (300 uL) was added dropwise keeping the temp below 10° C. The reaction was stirred for 12 h warming to room temperature. The reaction was diluted with ethyl acetate (75 mL) and 10% aq sodium carbonate (75 mL). The layers were separated and the organic layer was dried over magnesium sulfate, filtered and concentrated under reduced pressure. The material was purified using a biotage 40S cartridge eluting with hexanes:ethyl acetate (70:30) yielding 247 mg (40% yield) of a clear oil: ¹H NMR (400 MHz, CDCl₃); δ 7.73 (s, 1H), 7.37-7.20 (m, 7H), 7.12 (dt, J=7.1, 1.8 Hz, 1H), 4.02 (s, 2H), 3.56 (d, J=13.4 Hz, 1H), 3.48 (d, J=13.4 Hz, 1H), 2.95 (d, J=9.8 Hz, 1H), 2.80 (d, J=11.8 Hz, 1H), 2.71 (d, J=9.9 Hz, 1H), 2.10-2.00 (m, 2H), 1.91 (dt, J=12.8, 4.6 Hz, 1H), 1.85-1.65 (m, 2H), 1.29 (s, 9H).

3-(3-tert-Butyl-phenyl)-3-(2-chloro-acetylamino)-piperidine-1-carboxylic acid benzyl ester. To a stirred solution of N-[1-Benzyl-3-(3-tert-butyl-phenyl)-piperidin-3-yl]-2-chloro-acetamide (247 mg, 0.620 mmol) in Toluene (2 mL) was added benzylchloroformate (177 uL, 1.24 mmol). The reaction was heated to 80° C. and stirred for 4 h. An additional 2 eq was added and the reaction was stirred at room temperature for 3 days. The reaction was diluted with ethyl acetate (50 mL) and 10% aq sodium carbonate (50 mL). The layers were separated and the organic layer was dried over magnesium sulfate, filtered and concentrated under reduced pressure. The material was purified using a biotage 12i cartridge eluting with hexanes:ethyl acetate (70:30) yielding 240 mg (84% yield) of a clear oil: ¹H NMR (400 MHz, CDCl₃); δ 7.45-7.22 (m, 9H), 5.23 (d, J=12.3 Hz, 1H), 5.17 (d, J=12.3 Hz, 1H), 4.44-4.30 (m, 1H), 4.30-4.10 (m, 1H), 3.95-3.80 (m, 2H), 3.20-3.00 (m, 1H), 3.00-2.80 (m, 2H), 2.10-1.90 (m, 1H), 1.80-1.60 (m, 2H), 1.30 (s, 9H).

3-Amino-3-(3-tert-butyl-phenyl)-piperidine-1-carboxylic acid benzyl ester. The 3-(3-tert-butyl-phenyl)-3-(2-chloro-acetylamino)-piperidine-1-carboxylic acid benzyl ester (239 mg, 0.540 mmol) was taken up in ethanol (1 mL) and AcOH (200 uL) followed by the addition of thiourea (50 mg, 0.648 mmol). The reaction was heated to 80° C. and stirred for 12 h. The reaction was diluted with ethyl acetate (50 mL) and 10%aq sodium carbonate (50 mL). The layers were separated and the organic layer was dried over magnesium sulfate, filtered and concentrated under reduced pressure. The material was purified using a biotage 12i cartridge eluting with ethyl acetate: methanol (92:8) yielding 166 mg (84% yield) of a clear oil: retention time (min)=1.71, method [1]; MS(ESI) 367.4 (31), 350.4 (100).

### EXAMPLE 108

### PREPARATION OF EXAMPLE N-[3-[1-(3-TERT-BUTYL-PHENYL)-4-METHOXY-CYCLOHEXYLAMINO]-1-(3,5-DIFLUORO-BENZYL)-2-HYDROXY-PROPYL]-ACETAMIDE

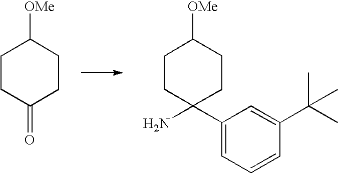

1-(3-tert-Butyl-phenyl)-4-methoxy-cyclohexylamine from 4-methoxycyclohexanone. 4-Methoxycyclohexanone was synthesized according to the procedure described in Kaiho, T. et al. J. Med. Chem. 1989, 32, 351-357. The ketone was converted to the 1-(3-tert-Butyl-phenyl)-4-methoxy-cyclohexylamine in the manner described in EXAMPLE 21, except using 1-bromo-3-tert-butyl-benzene in the first step yielding a 1:1 mixture of isomers: retention time (min)=1.33 and 1.42 (diastereomers), method [1], MS(ESI) 213.2 (M-NH₂); MS(ESI) 213.2 (M-NH₂).

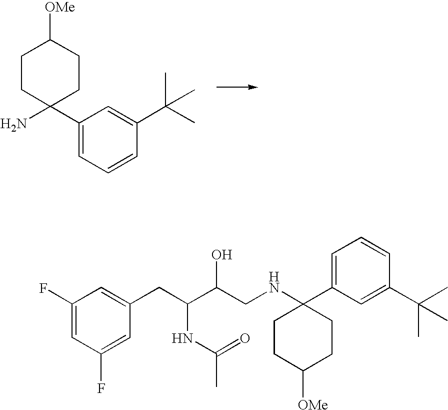

(1S, 2R)—N-[3-[1-(3-tert-Butyl-phenyl)-4-methoxy-cyclohexylamino]-1-(3,5-difluoro-benzyl)-2-hydroxy-propyl]-acetamide from 1-(3-tert-Butyl-phenyl)-4-methoxy-cyclohexylamine. The amine was converted into N-[3-[1-(3-tert-Butyl-phenyl)-4-methoxy-cyclohexylamino]-1-(3,5-difluoro-benzyl)-2-hydroxy-propyl]-acetamide according to the procedure described in EXAMPLE 22 yielding (1S,2R)—N-[3-[1-(3-tert-Butyl-phenyl)-4-methoxy-cyclohexylamino]-1-(3,5-difluoro-benzyl)-2-hydroxy-propyl]-acetamide as a mixture of two isomers, which were separated by flash chromatography (100% EtOAc to 15% MeOH/EtOAc gradient), and each was further purified by HPLC:

Isomer 1: retention time (min)=1.84, method [1]; ¹H NMR (300 MHz, CDCl₃); δ 7.57 (s,1H), 7.50-7.25 (m, 3H), 7.02 (d, J=9 Hz,1H), 6.68 (d, J=6 Hz, 2H), 6.60 (dt, J=9, 2 Hz, 1H), 6.42 (br s, 1H), 3.80-3.60 (m, 1H), 3.29 (s, 3H), 2.85 (dd, J=15, 6 Hz, 1H), 2.65 (dd, J=15, 9 Hz, 1H), 2.45 (d, J=6 Hz, 1H), 2.32-2.10 (m, 3H), 2.00-1.75 (m, 1H), 1.82 (s, 3H), 1.70-1.40 (m, 2H), 1.31 (s, 9H); ¹³C NMR (75 MHz, CDCl₃); δ 171.2, 171.0, 162.9 (dd, J=246.7, 12.9 Hz, 2C), 152.2, 142.2 (t, J=9.1 Hz, 1C), 128.6, 125.4, 124.1, 124.0, 112.0 (dd, J=17.1, 7.4 Hz, 2C), 101.9 (t, J=25.9 Hz, 1C), 74.9, 70.0, 60.4, 57.9, 55.4, 53.2, 44.5, 35.7, 34.9, 31.2, 29.8, 29.3, 26.6, 26.1, 22.8, 21.0, 14.1; MS (ESI) 503.2.

Isomer2: retention time (min)=1.78, method [1]; ¹H NMR (300 MHz, CDCl₃); δ 7.43 (s, 1H), 7.40-7.10 (m, 2H), 7.02 (d, J=9 Hz, 1H), 6.70-6.45 (m, 3H), 3.45-3.20 (m, 2H), 3.24 (s, 3H), 2.63 (dd, J=15, 6 Hz, 1H), 2.56 (dd, J=15, 9 Hz, 1H), 2.45-2.20 (m, 4H), 1.97 (s, 1.5H), 1.95-1.80 (m, 2H), 1.78 (s, 1.5H), 1.72-1.60 (m, 2H), 1.60-1.40 (m, 2H), 1.27 (s, 9H); ¹³C NMR (75 MHz, CDCl₃); δ 171.1, 170.6, 162.7 (dd, J=246.7, 12.9 Hz, 2C), 151.1, 142.1 (t, J=9.1 Hz, 1C), 128.0, 123.7, 123.6, 123.4, 111.7 (dd, J=17.1, 7.4 Hz, 2C), 101.6 (t, J=25.9 Hz, 1C), 70.1, 60.2, 57.9, 55.4, 53.4, 43.6, 36.1, 34.6, 31.9, 31.6, 31.2, 26.6, 22.8, 20.8, 13.9; MS (ESI) 503.2.

### EXAMPLE 109

### EXAMPLE N-(4-(1-(3-TERT-BUTYLPHENYL)-4-(TRIFLUOROMETHYL)CYCLOHEXYLAMINO)-1-(3,5-DIFLUOROPHENYL)-3-HYDROXYBUTAN-2-YL)ACETAMIDE

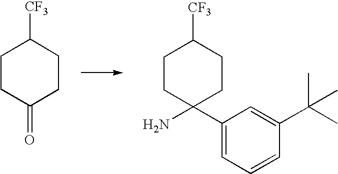

1-(3-tert-Butyl-phenyl)-4-trifluoromethyl-cyclohexylamine from 4-Trifluoromethyl-cyclohexanone. 4-Trifluoromethylcyclohexanone (Matrix Scientific) was converted to the titled amine by the method described in EXAMPLE 21. Retention time (min)=1.64 and 1.69 (diastereomers), method [1]; ¹H NMR (300 MHz, CDCl₃); δ 7.55 (s, 0.5H), 7.47 (s, 0.5H), 7.40-7.20 (m, 3H), 2.54 (d, J=13.2 Hz, 1H), 2.15 (br s, 2H), 2.00-1.80 (m, 4H), 1.75-1.50 (m, 4H), 1.34 (s, 9H); MS(ESI) 283.1 (M-NH₂).

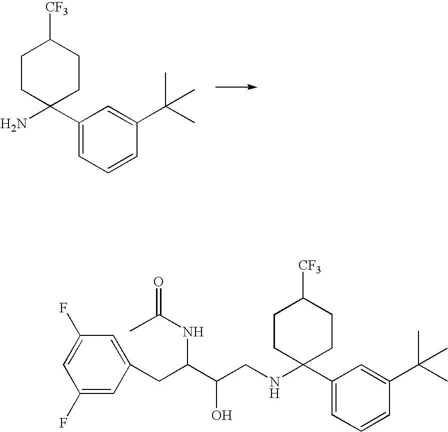

N-[3-[1-(3-tert-Butyl-phenyl)-4-trifluoromethyl-cyclohexylamino]-1-(3,5-difluoro-benzyl)-2-hydroxypropyl]-acetamide from 1-(3-tert-Butyl-phenyl)-4-trifluoromethyl-cyclohexylamine. The titled compound was synthesized from the intermediate amine by the route described in EXAMPLE 22. The diastereomers were separated by flash chromatography (EtOAc/hexanes elution), and further purified by HPLC yielding each as the trifluoroacetic acid salt.

Isomer 1: R_{f}=0.66 (4:1 EtOAc/hexanes); retention time (min)=2.017, method [1]; ¹H NMR (300 MHz, CDCl₃); δ 7.42 (s, 1H), 7.40-7.20 (m, 2H), 7.20-7.10 (m, 1H), 6.75-6.55 (m, 3H), 5.63 (d, J=8.7 Hz, 1H), 4.22-4.02 (m, 1H), 3.37 (q, J=3.5 Hz, 1H), 2.89 (dd, J=13.5, 4.5 Hz, 1H), 2.75 (dd, J=13.5, 8.1 Hz, 1H), 2.30-2.00 (m, 4H), 1.88 (s, 3H), 1.85-1.50 (m, 7H), 1.32 (s, 9H); MS (ESI) 541.2.

Isomer 2: R_{f}=0.11 (4:1 EtOAc/hexanes); retention time (min)=2.005, method [1]; ¹H NMR (300 MHz, CDCl₃); δ 8.05 (br s, 1H), 7.35-7.20 (m, 2H), 7.20-7.12 (m, 1H), 6.72-6.55 (m, 3H), 5.57 (d, J=9.0 Hz, 1H), 4.15-3.90 (m, 1H), 3.30-3.10 (m, 1H), 2.82 (dd, J=13.5, 5.1 Hz, 1H), 2.67 (dd, J=13.5, 8.1 Hz, 1H), 2.65-2.50 (m, 2H), 2.40-2.00 (m, 5H), 1.85 (s, 3H), 1.75-1.40 (m, 4H), 1.32 (s, 9H); MS (ESI) 541.2. ¹³C NMR (75 MHz, MeOD-d₄) δ 172.3, 164.4 (dd, J=246.7, 13.1 Hz, 2C), 162.1, 154.3, 144.1 (t, J=9.1 Hz, 1C), 133.6, 130.6, 128.0, 126.4, 126.0, 112.8 (dd, J=17.1, 7.4 Hz, 2C), 102.7 (t, J=25.9 Hz, 1C), 70.5, 64.6, 54.7, 46.2, 36.8, 36.0, 33.2, 31.7, 31.5, 22.5, 22.3.

### EXAMPLE 110

### SYNTHESIS OF EXAMPLE N-[3-[1-(6-TERT-BUTYL-PYRIMIDIN-4-YL)-CYCLOHEXYLAMINO]-1-(3,5-DIFLUORO-BENZYL)-2-HYDROXY-PROPYL]-ACETAMIDE

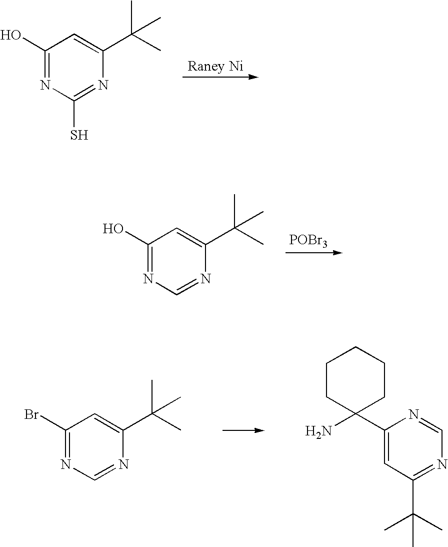

Synthesis of 1-(6-tert-Butyl-pyrimidin-4-yl)-cyclohexylamine 6-tert-Butyl-pyrimidin-4-ol from 6-tert-Butyl-2-mercapto-pyrimidin-4-ol. Procedure adapted from: J. Med. Chem. 2002, 45, 1918-1929. 6-tert-Butyl-2-mercapto-pyrimidin-4-ol (1.0 g, 5.4 mmol), synthesized according to the procedure described in J. Am. Chem. Soc. 1945, 67, 2197, was dissolved in boiling EtOH (30 mL). Raney Ni 2800 slurry (Aldrich) was added to the mixture dropwise until starting material had been determined by TLC to be completely consumed (approx. 5 mL of slurry over 3 h). The mixture was filtered through diatomaceous earth, washed with EtOH (50 mL). The filtrate was concentrated under reduced pressure yielding 794 mg, 96% of desired product: Rf =0.13 (1:1 EtOAc/hexanes); ¹H NMR (300 MHz, MeOD-d₄) δ 8.14 (s, 1H), 6.37 (s, 1H), 1.29 (s, 9H).

4-Bromo-6-tert-butyl-pyrimidine from 6-tert-Butyl-pyrimidin-4-ol. Procedure adapted from: Kim, J. T. Org. Lett. 2002, 4, 4697-4699. Phosphorus oxybromide (14.9 g, 51.9 mmol) was added to a solution of 6-tert-Butyl-pyrimidin-4-ol (5.2 g, 34 mmol) and N,N-dimethylaniline (1.25 g, 10 mmol) in anhydrous benzene (150 mL). The mixture was then heated to reflux for 3 h. The reaction mixture was then allowed to cool to rt, and saturated Na₂CO₃ (200 mL) was added. The layers were separated, and the aqueous further extracted with EtOAc (300 mL). The combined organic layers were washed (sat'd NaCl), dried (sodium sulfate), filtered and concentrated under reduced pressure. Flash chromatography (0-20% EtOAc/hexanes gradient elution) afforded pure product (3 g, 40%): R_{f}=0.84 (1:4 EtOAc/hexanes); ¹H NMR (300 MHz, CDCl₃); δ 8.82 (d, J=0.6 Hz,1H), 7.74 (d, J=0.6 Hz,1H), 1.35 (s, 9H).

1-(6-tert-Butyl-pyrimidin-4-yl)-cyclohexylamine from 4-Bromo-6-tert-butyl-pyrimidine. The cyclohexylamine was synthesized from the aryl bromide according to the method described in EXAMPLE 59, using 2-methylpropane-2-sulfinic acid cyclohexylideneamide prepared according to the method of Liu, G. et al. J. Org. Chem. 1999, 64,1278-1284: retention time (min)=1.48, method [1]; MS (ESI) 234.2.

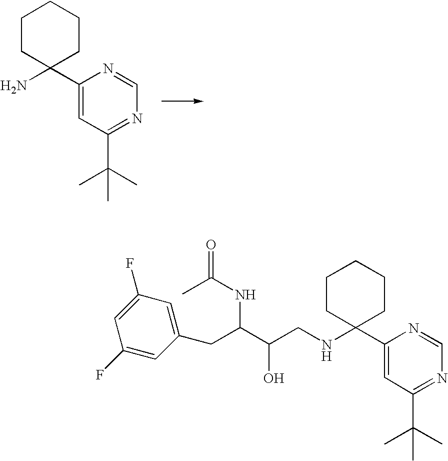

N-[3-[1-(6-tert-Butyl-pyrimidin-4-yl)-cyclohexylamino]-1-(3,5-difluoro-benzyl)-2-hydroxy-propyl]acetamide from 1-(6-tert-Butyl-pyrimidin-4-yl)-cyclohexylamine. The compound was synthesized from the intermediate amine according to methods described in EXAMPLE 22 yielding (1S,2R)-N-[3-[1-(6-tert-Butyl-pyrimidin-4-yl)-cyclohexylamino]-1-(3,5-difluoro-benzyl)-2-hydroxy-propyl]acetamide, which was purified by HPLC yielding the trifluoroacetic acid salt: retention time (min)=1.67, method [1]; ¹H NMR (300 MHz, CDCl₃); δ 9.20 (d, J=1.2 Hz, 1H), 7.67 (d, J=1.2 Hz, 1H), 6.73 (dd, J=8.2, 2.2 Hz, 2H), 6.67 (dt, J=9.0, 2.2 Hz, 1H), 6.05 (d, J=8.6 Hz, 1H), 4.20-4.05 (m, 1H), 3.85-3.70 (m, 1H), 3.12 (dd, J=14.7, 4.5 Hz, 1H), 2.90-2.70 (m, 1H), 2.63 (dd, J=12.4, 6.2 Hz, 1H), 2.55-2.30 (m, 1H), 2.25-1.75 (m, 8H), 1.92 (s, 3H), 1.39 (s, 9H); MS (ESI) 475.2.

### EXAMPLE 111

### PREPARATION OF N-[3-[1-(3-TERT-BUTYL-PHENYL)-4-OXO-CYCLOHEXYLAMINO]-1-(3,5-DIFLUORO-BENZYL)-2-HYDROXYL-PROPYL]-ACETAMIDE

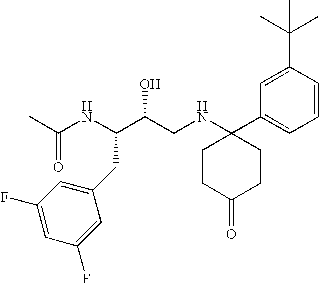

This compound was synthesized according to the procedure in EXAMPLE 26, except using 8-(3-tert-Butyl-phenyl)-1,4-dioxa-spiro[4.5]dec-8-ylamine, which was synthesized according to the method of EXAMPLE 25. HPLC purification afforded trifluoroacetic acid salt: retention time (min)=1.63, method [1]; ¹H NMR (300 MHz, CDCl₃): δ 7.70 (s, 1H), 7.52 (d, J=7 Hz, 1H), 7.46 (t, 1H), 7.38 (d, J=7 Hz, 1H), 6.68 (d, J=7 Hz, 2H), 6.63 (d, J=2 Hz, 1H), 6.03 (d, J=8 Hz, 1H), 4.06-4.02 (m, 1H), 3.74 (m, 1H), 2.96 (d, J=4 Hz, 1H), 2.79-2.67 (m, 3H), 2.55-2.53 (m, 5H), 2.32-2.28 (m, 1H), 1.84 (s, 3H), 1.33 (s, 9H); ¹³C NMR (75MHz, CDCl₃); δ 170.2, 163.7 (dd, J=246.7, 13.1 Hz, 2C), 151.3, 143.1, 141.6, 128.1, 123.9, 122.9, 122.6, 112.01 (dd, J=16.7, 12.2 Hz, 2C), 102.0 (t, J=25.4 Hz, 1C), 71.1, 65.7, 56.4, 52.4, 43.8, 37.1, 37.0, 36.1, 35.4, 35.3, 34.7, 31.2, 23.0, 15.0; MS (ESI) 487.2 (M+H), 509.2 (M+Na).

### EXAMPLE 112

### PREPARATION OF N-[3-4-ACETYLAMINO-1-(3-TERT-BUTYL-PHENYL)-CYCLOHEXYLAMINO]-1-(3,5-DIFLUORO-BENZYL)-2-HYDROXY-PROPYL]-ACETAMIDE

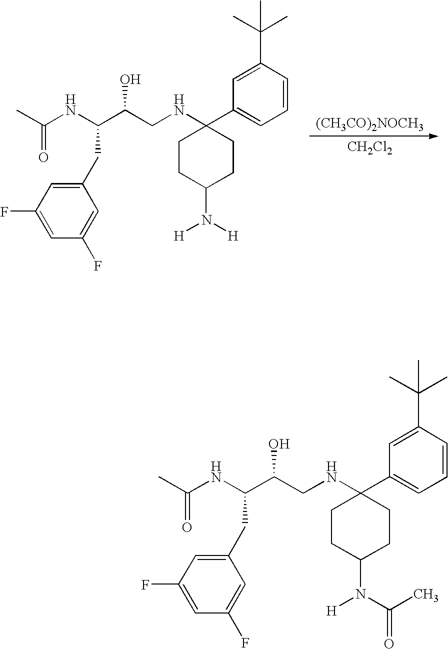

To a solution of N-[3-[4-amino-1-(3-tert-butyl-phenyl)-cyclohexylamino]-1-(3,5-difluoro-benzyl)-2-hydroxyl-propyl]-acetamide (0.218 g, 0.433 mmol) in anhydrous CH₂Cl₂ (3 mL) was added N,N-diacetyl-O-methylhydroxylamine (0.03 mL, 0.256 mmol). The reaction mixture was stirred at room temperature, overnight under, N₂(g) inlet prior to quenching with H₂O. The mixture was extracted with CH₂Cl₂ and then the organic layer was collected, dried over anhydrous sodium sulfate, filtered and concentrated. The crude product was purified by HPLC and hydrolyzed yielding the parent compound: retention time (min)=1.57, method [1]; ¹H NMR (300 MHz, CD₃OD); δ 7.60 (s, 1H), 7.39 (broad s, 3H), 6.77 (d, J=7 Hz, 2H), 6.74 (d, J=2 Hz, 1H), 3.92-3.77 (m, 3H), 3.57-3.47 (m, 2H), 3.06 (d, J=14 Hz, 1H), 2.73-2.69 (m, 2H), 2.63-2.37 (m, 3H), 1.84 (s, 3H), 1.61 (s, 3H), 1.34 (s, 9H); MS (ESI) 530.5 (M+H).

### EXAMPLE 113

### PREPARATION OF N-[3-[1-(3-TERT-BUTYL-PHENYL)-4-CYANOMETHYLENE-CYCLOHEXYLAMINO]-1-(3,5-DIFLUORO-BENZYL)-2-HYDROXY-PROPYL]-ACETAMIDE

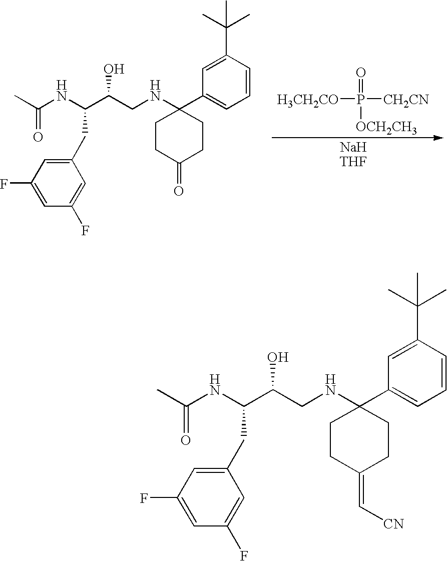

To a solution of diethyl(cyanomethyl)phosphonate (0.05 mL, 0.309 mmol) in anhydrous THF (0.7 mL) was added a 60% dispersion of sodium hydride in mineral oil (0.008 g, 0.20 mmol). Vigorous gas evolution was observed while stirring at room temperature under N₂(g) inlet. After 1.5 h a solution of N-[3-[1-(3-tert-butyl-phenyl)-4-oxo-cyclohexylamino]-1-(3,5-difluoro-benzyl)-2-hydroxyl-propyl]-acetamide (0.119 g, 0.123 mmol) in anhydrous THF (0.5 mL) was added to the reaction flask. The mixture was allowed to stir overnight. The reaction was quenched with H₂O and extracted with CH₂Cl₂. The organic layer was collected, dried over anhydrous sodium sulfate, filtered and concentrated. The crude product was purified by HPLC yielding the trifluoroacetic acid salt: retention time (min)=1.81, method [1]; MS (ESI) 510.2 (M+H).

### EXAMPLE 114

### PREPARATION OF [4-[3-ACETYLAMINO-4-(3,5-DIFLUORO-PHENYL)-2-HYDROXY-BUTYLAMINO]-4-(3-TERT-BUTYL-PHENYL)-CYCLOHEXYLIDENE]-ACETIC ACID METHYL ESTER

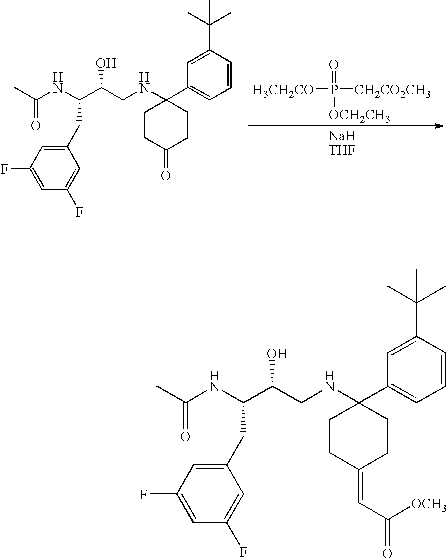

To a solution of methyl diethylphosphonoacetate (0.20 mL, 1.102 mmol) in anhydrous THF (1 mL) was added a 60% dispersion of sodium hydride in mineral oil (0.80 g, 20.0 mmol). Vigourous gas evolution was observed while stirring at room temperature under N₂(g) inlet. After 2 h a solution of N-[3-[1-(3-tert-butyl-phenyl)-4-oxo-cyclohexylamino]-1-(3,5-difluoro-benzyl)-2-hydroxyl-propyl]-acetamide (0.291 g, 0.598 mmol) in anhydrous THF (1 mL) was added to the reaction flask. The mixture was allowed to stir for 2 days. The reaction was quenched with H₂O and extracted with CH₂Cl₂. The organic layer was collected, dried over anhydrous sodium sulfate, filtered and concentrated. The crude product was purified by flash chromatography, eluting with 5% MeOH in CH₂Cl₂ yielding 0.085 g (0.157 mmol, 26%) of the product. HPLC purification afforded the desired product: retention time (min)=1.87, method [1]; ¹H NMR (300 MHz, CD₃OD); δ 7.56 (s, 1H), 7.31-7.26 (m, 1H), 7.24-7.26 (m, 2H), 6.70 (d, J=7 Hz, 3H), 3.80-3.78 (m, 1H), 3.70 (s, 3H), 3.36-3.33 (m, 1H), 2.83-2.77 (m, 3H), 2.51 (t, 1H), 2.30 (d, J=4 Hz, 1H), 2.24 (d, J=10 Hz, 1H), 2.16-2.07 (m, 1H), 1.95-1.86 (m, 7H), 1.71 (s, 3H), 1.33 (s, 9H); MS (ESI) 543.2 (M+H).

### EXAMPLE 115

### PREPARATION OF N-[3-[1-(3-TERT-BUTYL-PHENYL)-4-(3-METHYL-UREDIO)-CYCLOHEXYLAMINO]-1-(3,5-DIFLUORO-BENZYL)-2-HYDROXY-PROPYL]-ACETAMIDE

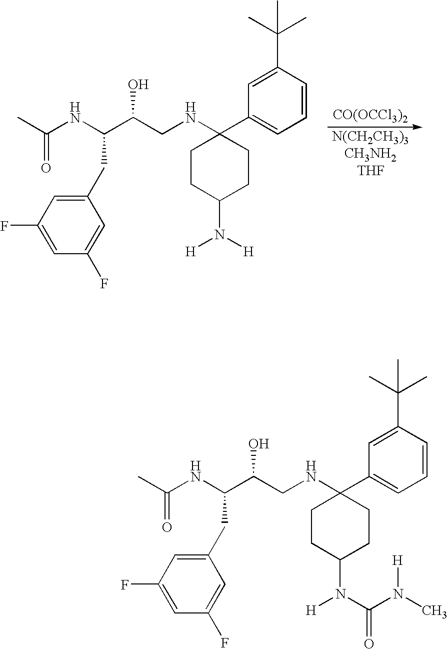

Procedure adapted from: Synthesis, 2000, 10, 1449-1453. To a solution of triphosgene (0.036 g, 0.121 mmol) in anhydrous THF (4 mL) was added a solution of N-[3-[4-amino-1-(3-tert-butyl-phenyl)-cyclohexylamino]-1-(3,5-difluoro-benzyl)-2-hydroxyl-propyl]-acetamide (0.121 g, 0.248 mmol) and triethylamine (0.08 mL, 0.574 mmol) in anhydrous THF (1 mL). The mixture was allowed to stir at room temperature under N₂(g) inlet for 1 h. A 2.0 M solution of methylamine (0.22 mL, 0.44 mmol) and triethylamine (0.035 mL, 0.251 mmol) in anhydrous THF (1 mL) was added to the reaction flask and stirred for 1 h. The mixture was concentrated and purified by HPLC yielding the diastereomers:

Isomer 1: retention time (min)=1.58, method [1]; ¹H NMR (300 MHz, CD₃OD); δ 7.50 (s, 1H), 7.29 (s, 1H), 6.75 (d, J=8 Hz, 2H), 6.74 (t, 1H), 3.95-3.89 (m, 1H), 3.64-3.63 (m, 1H), 3.41-3.33 (m, 1H), 2.98 (d, J=14 Hz, 1H), 2.64 (s, 3H), 2.54-2.46 (m, 3H), 2.35-2.22 (m, 2H), 1.90-1.86 (m, 2H), 1.77-1.72 (m, 5H), 1.32 (s, 9H); MS (ESI) 545.3 (M+H)

Isomer2: retention time (min)=1.63, method [1]; ¹H NMR (300 MHz, CD₃OD); δ 7.52 (s, 1H), 7.24 (s, 1H), 6.81 (d, J=8 Hz, 2H), 6.77 (t, 1H), 4.13-4.07 (m, 1H), 3.48-3.43 (m, 2H), 3.08 (d, J=14 Hz, 1H), 2.69 (s, 3H), 2.59 (t,1H), 2.28-2.19 (m, 2H), 2.06-1.99 (m, 2H), 1.91-1.79 (m, 9H), 1.75 (s, 9H); MS (ESI) 545.3 (M+H).

### EXAMPLE 116

### PREPARATION OF N-[3-[1-(3-TERT-BUTYL-PHENYL)-4-METHANESULFONYLAMINO-CYCLOHEXYLAMINO]-1-(3,5-DIFLUORO-BENZYL)-2-HYDROXY-PROPYL]-ACETAMIDE

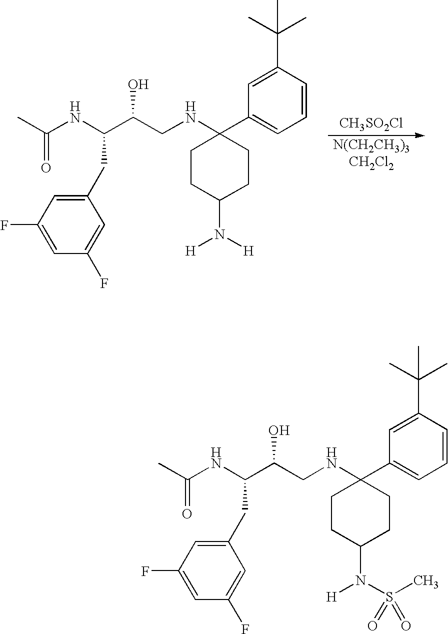

To a solution of N-[3-[4-amino-1-(3-tert-butyl-phenyl)-cyclohexylamino]-1-(3,5-difluoro-benzyl)-2-hydroxyl-propyl]-acetamide (0.106 g, 0.217 mmol) in anhydrous CH₂Cl₂ (1 mL) was added triethylamine (0.03 mL, 0.215 mmol). The reaction mixture was cooled to 0° C. prior to addition of methansulfonyl chloride (0.02 mL, 0.257 mmol) and then allowed to stirred under N₂(g) inlet overnight. Upon completion, the reaction was quenched with H₂O and extracted with EtOAc followed by washing with saturated NaCl (aq). The organic layer was collected, dried over anhydrous sodium sulfate, filtered and concentrated. The crude product was purified by HPLC yielding the diastereomers: retention time (min)=1.62, method [1]; MS (ESI) 566.2 (M+H) and retention time (min)=1.73, method [1]; ¹H NMR (300 MHz, CD₃OD); δ 7.52 (s, 1H), 7.24 (s, 3H), 6.80 (d, J=9 Hz, 2H), 6.78-6.70 (m, 1H), 4.11-4.05 (m, 1H), 3.46-3.42 (m, 1H), 3.08 (d, J=14 Hz, 1H), 2.97 (s, 3H), 2.60-2.52 (m, 1H), 2.28-2.19 (m, 2H), 2.04-2.03 (m, 2H), 1.88-1.79 (m, 6H), 1.76 (s, 3H), 1.32 (s, 9H); MS (ESI) 566.2 (M+H).

### EXAMPLE 117

### PREPARATION OF N-[3-[1-(3-TERT-BUTYL-PHENYL)-4-FORMYLAMINO-CYCLOHEXYLAMINO]-1-(3,5-DIFLUORO-BENZYL)-2-HYDROXY-PROPYL]-ACETAMIDE

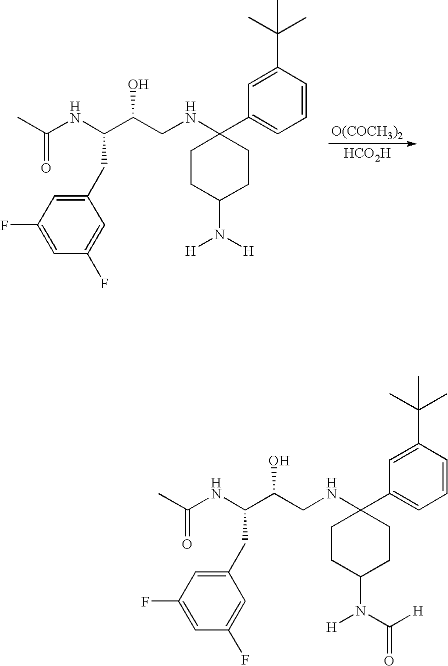

Procedure adapted from: J. Med. Chem. 1990, 33(1), 187-196. Acetic anhydride (0.11 mL, 1.166 mmol) was added to a solution of N-[3-[4-amino-1-(3-tert-butyl-phenyl)-cyclohexylamino]-1-(3,5-difluoro-benzyl)-2-hydroxyl-propyl]-acetamide (0.100 g, 0.205 mmol) in formic acid (0.37 mL, 8.70 mmol) cooled to 0° C. The reaction mixture was stirred under N₂(g) inlet while warming to RT overnight. LC/MS results indicated that the reaction was 50% complete. Therefore, the reaction mixture was then subjected to additional equivalents of acetic anhydride (0.11 mL, 1.166 mmol) and formic acid (0.37 mL, 8.70 mmol) and placed in an oil bath at 45° C. with condenser under N₂(g) inlet overnight. The reaction solvents were removed in vacuo. The crude product was dissolved in CHCl₃ and washed with H₂O followed by saturated NaCl (aq). The organic layer was collected, dried over anhydrous sodium sulfate, filtered and concentrated. Purification by HPLC afforded the desired compound: retention time (min)=1.64, method [1]; ¹H NMR (300 MHz, CD₃OD); δ 7.52 (s, 1H), 7.24 (d, J=6 Hz, 3H), 6.80-6.71 (m, 3H), 4.10-4.06 (m, 1H), 3.46-3.32 (m, 1H), 3.07 (d, J=10 Hz, 1H), 2.97 (s, 1H), 2.60-2.56 (m, 1H), 2.28-2.23 (m, 2H), 2.04-2.03 (m, 2H), 1.88-1.79 (m, 5H), 1.76 (d, J=6Hz, 4H), 1.32 (d, J=5Hz, 9H); MS (ESI) 516.2 (M+H).

### EXAMPLE 118

### PREPARATION OF 2-[4-[3-ACETYLAMINO-4-(3,5-DIFLUORO-PHENYL)-2-HYDROXY-BUTYLAMINO]-4-(3-TERT-BUTYL-PHENYL)-CYCLOHEXYLIDENE]-N,N-DIMETHYL-ACETAMIDE

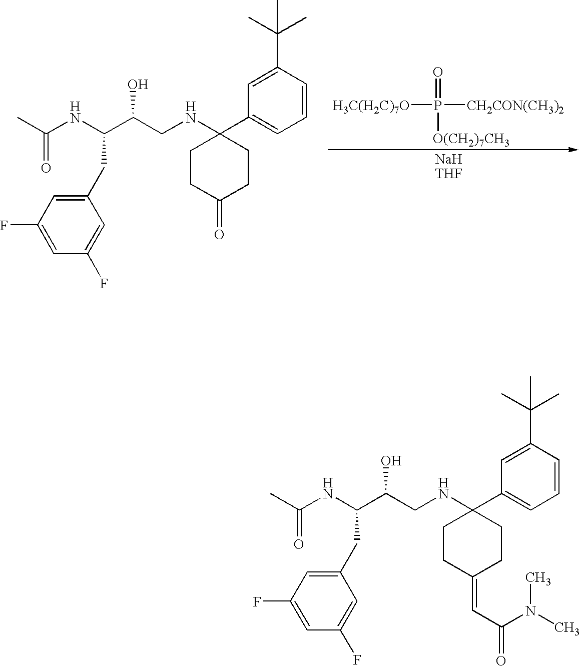

To a solution of dioctyl (N,N-dimethylcarbamoylmethyl)phosphonate (0.05 g, 0.128 mmol) in anhydrous THF (1 mL) was added a 60% dispersion of sodium hydride in mineral oil (0.026 g, 0.65 mmol). Vigourous gas evolution was observed while stirring at room temperature under N₂(g) inlet. After 3.5 h a solution of N-[3-[1-(3-tert-butyl-phenyl)-4-oxo-cyclohexylamino]-1-(3,5-difluoro-benzyl)-2-hydroxyl-propyl]-acetamide (0.041 g, 0.084 mmol) in anhydrous THF (1 mL) was added to the reaction flask. The mixture was allowed to stir overnight. The reaction was quenched with H₂O and extracted with CH₂Cl₂. The organic layer was collected, dried over anhydrous sodium sulfate, filtered and concentrated. HPLC purification afforded the parent compound: retention time (min)=1.83, method [1]; ¹H NMR (300 MHz, CD₃OD); δ 7.53 (s, 1H), δ 7.24 (m, 1H), 7.22 (d, J=4 Hz, 2H), 6.68 (d, J=8 Hz, 3H), 3.78 (m, 1H), 3.41 (m, 1H), 3.12 (s, 3H), 2.94 (s, 3H), 2.84 (s, 2H), 2.79 (d, J=15 Hz, 1H), 2.49 (t, 1H), 2.32-2.22 (m, 2H), 2.06 (m, 2H), 1.85 (m, 6H), 1.64 (s, 3H), 1.29 (s, 9H); MS (ESI) 556.3 (M+H).

### EXAMPLE 119

### PREPARATION OF [4-[3-ACETYLAMINO-4-(3,5-DIFLUORO-PHENYL)-2-HYDROXY-BUTYLAMINO]-4-(3-TERT-BUTYL-PHENYL)-CYCLOHEXYL]-CARBAMIC ACID METHYL ESTER

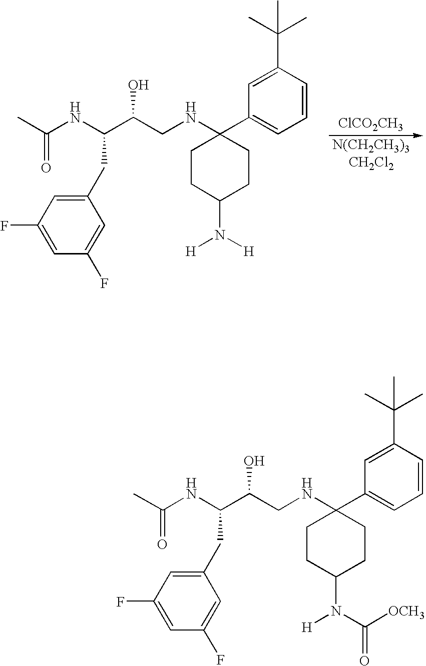

To a solution of N-[3-[4-amino-1-(3-tert-butyl-phenyl)-cyclohexylamino]-1-(3,5-difluoro-benzyl)-2-hydroxyl-propyl]-acetamide (0.097 g, 0.199 mmol) in anhydrous CH₂Cl₂ (1 mL) was added triethylamine (0.03 mL, 0.215 mmol). The reaction mixture was cooled to 0° C. prior to addition of methyl chloroformate (0.0154 mL, 0.199 mmol) and then allowed to stirred under N₂(g) inlet overnight. Upon completion, the reaction was quenched with H₂O and extracted with EtOAc followed by washing with saturated NaCl (aq). The organic layer was collected, dried over anhydrous sodium sulfate, filtered and concentrated. The crude product was purified by HPLC yielding two diastereomers:

Isomer 1: retention time (min)=1.73, method [1]; ¹H NMR (300 MHz, CD₃OD); δ 7.47 (s, 1H), 7.25 (s, 3H), 6.72 (d, J=8 Hz, 3H), 3.89-3.87 (m, 1H), 3.54-3.51 (m, 4H), 2.93 (d, J=14 Hz, 1H), 2.51-2.43 (m, 3H), 2.30-2.15 (m, 2H), 1.91-1.79 (m, 2H), 1.73-1.64 (m, 5H), 1.29 (s, 9H); MS (ESI) 546.3 (M+H).

Isomer 2: retention time (min)=1.79, method [1]; ¹H NMR (300 MHz, CD₃OD); δ 7.50 (s, 1H), 7.22 (s, 3H), 6.77 (d, J=8 Hz, 2H), 6.72 (t,1H), 4.06 (m, 1H), 3.60 (s, 3H), 3.41-3.35 (m, 2H), 3.04 (d, J=14 Hz, 1H), 2.58-2.49 (m, 1H), 2.29-2.16 (m, 2H), 2.06-2.01 (m, 2H), 1.87-1.63 (m, 8H), 1.29 (s, 9H); MS (ESI) 546.3 (M+H).

### EXAMPLE 120

### PREPARATION OF N-[3-[4-(ACETYL-HYDROXY-AMINO)-1-(3-TERT-BUTYL-PHENYL)-CYCLOHEXYLAMINO]-1-(3,5-DIFLUORO-BENZYL)-2-HYDROXY-PROPYL]-ACETAMIDE

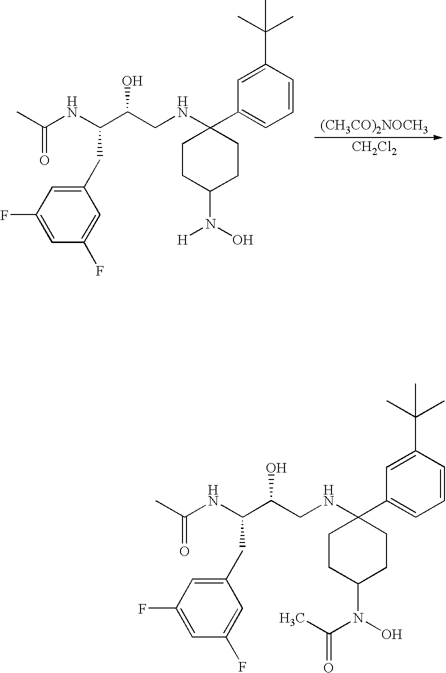

To a solution of N-[3-[1-(3-tert-butyl-phenyl)-4-hydroxyamino-cyclohexylamino]-1-(3,5-difluoro-benzyl)-2-hydroxy-propyl]-acetamide (0.218 g, 0.433 mmol) in anhydrous CH₂Cl₂ (3 mL) was added N,N-diacetyl-O-methylhydroxylamine (0.06 mL, 0.512 mmol). The reaction mixture was stirred at room temperature overnight under N₂(g) inlet prior to quenching with H₂O. The mixture was extracted with CH₂Cl₂ and then the organic layer was collected, dried over anhydrous sodium sulfate, filtered and concentrated. The crude product was purified by HPLC yielding a trifluoroacetic acid salt: retention time (min)=1.603, method [1]; ¹H NMR (300 MHz, CD₃OD); δ 7.65 (s, 1H), 7.55 (t, 1H), 7.47 (d, J=8 Hz, 2H), 6.77-6.73 (m, 3H), 4.46-4.44 (m, 1H), 3.85-3.77 (m, 1H), 3.48-3.54 (m, 1H), 3.17 (d, J=14 Hz, 1H), 2.91-2.87 (m, 2H), 2.62 (broad s, 2H), 2.54 (t, 1H), 2.08-1.92 (m, 5H), 1.73-1.62 (m, 5 H), 1.58-1.54 (m, 1H), 1.35 (s, 9H); MS (ESI) 546.4 (M+H).

### EXAMPLE 121

### PREPARATION OF 1-(3-TERT-BUTYL-5-FLUORO-PHENYL)-CYCLOHEXYLAMINE HYDROCHLORIDE SALT

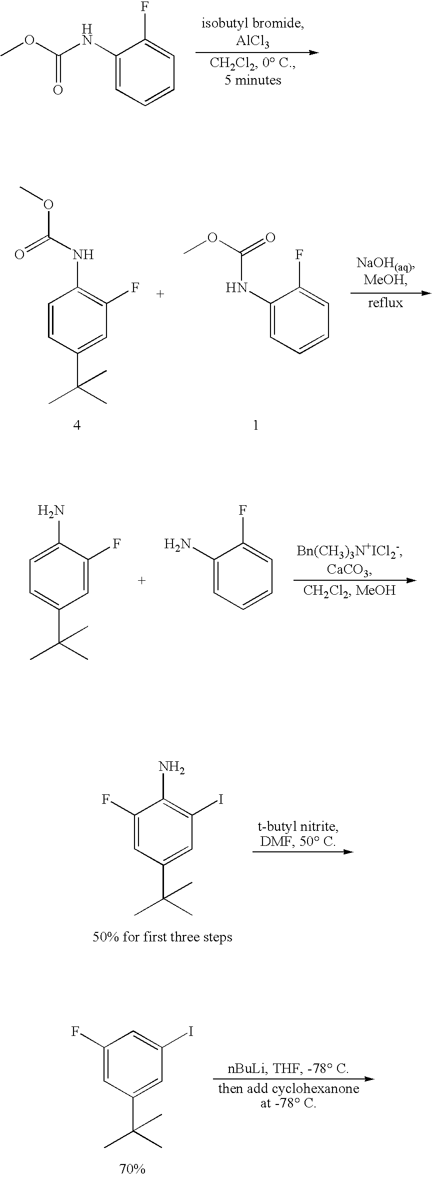

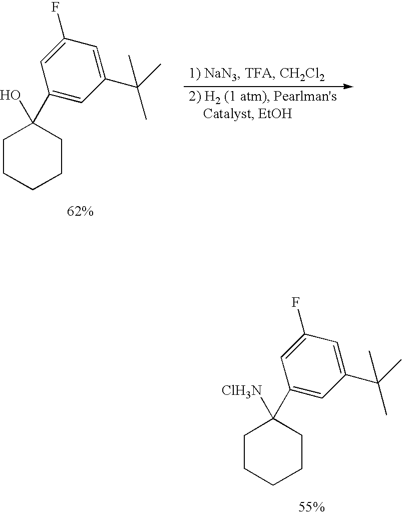

(4-tert-Butyl-2-fluoro-phenyl)-carbamic acid methyl ester: To a stirred solution of the carbamate (12.2 gm, 72 mmol) in 144 mL dichloromethane at 0° C. under a drying tube was added aluminum trichloride (28.85 gm, 216 mmol) carefully portion wise as a solid (some exotherm). The suspension was allowed to cool back to 0° C. for about 5 min and then isobromobutane (39.22 mL, 360 mmol) was added carefully by syringe at a rate that avoided reflux. The reaction was stirred for 5 min. HPLC shows near complete conversion at this time (retention time (min)=3.60, method [8]). The reaction was carefully poured into rapidly stirring ice water (500 mL) and diluted with 400 mL CH₂Cl₂. The mixture was stirred for about 5 min and the layers separated. The organics were washed 2×100 mL with H₂O, 1×200 mL with saturated NaHCO₃ and 1×100 mL with brine. The organics were dried (magnesium sulfate), filtered and concentrated to a brown oil that was used crude in the next reaction.

4-tert-Butyl-2-fluoro-phenylamine: To a stirred solution of the crude carbamate (18.4 g, 81.7 mmol) in 163 mL MeOH at room temperature under nitrogen was added 2 N NaOH (81.7 mL, 163.4 mmol). The reaction was warmed to 75° C. and stirred overnight. 40 mL of 2 N NaOH was added and the reaction stirred at 75° C. overnight again. HPLC showed a completed reaction (retention time=3.59, 3.65, method [8]). The reaction was cooled to room temperature and most of the MeOH was removed by rotovap. The residual aqueous mixture was cooled on ice and neutralized to pH=8 with conc. HCl. The solution was then extracted 2×100 mL with CH₂Cl₂ and the organics combined, dried (MgSO₄), filtered and concentrated to a brown oil which was taken into the iodination step as is.

4-tert-Butyl-2-fluoro-6-iodo-phenylamine: To a stirred solution of the crude aniline (12.8 g, 76.54 mmol) in 240 mL CH₂Cl₂ and 80 mL MeOH at room temperature under nitrogen was added calcium carbonate (15.32 g, 153.1 mmol) followed by the iodinating reagent, benzyltrimethylammonium iododichloride (67.28 g, 153.1 mmol). The reaction was allowed to precede overnight at room temperature. HPLC showed complete consumption of starting material and a new late eluting peak. The reaction was diluted to 500 mL with CH₂Cl₂ and poured into ice cold 10% NaHSO₃ with rapid stirring. The layers were separated and the organics washed 1×500 mL with 10% NaHSO₃, 1×500 mL with H₂O and 1×500 mL with saturated NaHCO₃. The organics were dried (magnesium sulfate), filtered and concentrated to a brown oil which was diluted in CH₂Cl₂ and absorbed onto silica gel. After rotovap and thorough high vacuum drying the silica was loaded into a ZIF module in line with a Biotage 75S column and eluted first with pure hexanes and then 98/2 hexanes/Et₂O. The product was isolated and concentrated to a brown oil (11.72 g, 52% for three steps); retention time (min)=4.45, method [8]; ¹H NMR (400 MHz, CDCl₃); δ 7.38 (s, 1H), 7.00 (d, J=10.8 Hz, 1H), 3.99 (s, 2H), 1.25 (s, 9H).

1-tert-Butyl-3-fluoro-5-iodo-benzene: To a stirred solution of t-butyl nitrite (7.13 mL, 60 mmol) in 80 mL DMF at 60° C. under nitrogen was added a solution of the iodoaniline (11.72 g, 40 mmol) in 80 mL DMF dropwise by cannulation. The reaction began to evolve gas. After complete addition the reaction was stirred for 1 h and then cooled to room temperature. HPLC showed complete consumption of starting material and a new late eluting peak. The reaction was diluted with 1 L EtOAc and washed 4×800 mL with H₂O and then 1×800 mL with brine. The organics were dried (magnesium sulfate), filtered and concentrated to a brown oil that was loaded onto a Biotage 65 column with hexane and eluted with the same solvent. The product containing fractions were pooled and partially concentrated to about 100 mL. The solution of combined fractions was washed 1×100 mL with 10% NaHSO₃, 1×100 mL with H₂O and 1×100 mL with NaHCO₃. The clear organics were dried (magnesium sulfate), filtered and concentrated to a clear oil (6.8 g, 61%); ¹H NMR (400 MHz, CDCl₃); δ 7.48 (s, 1H), 7.27-7.22 (m,1H), 7.04 (d, J=10.5 Hz,1H), 1.26 (s, 9H).

1-(3-tert-Butyl-5-fluoro-phenyl)-cyclohexanol: To a stirred solution of the iodobenzene derivative (2.3 g, 8.27 mmol) in 16 mL THF at −78° C. under nitrogen was added n-BuLi (2.5 M in hexanes, 3.31 mL, 8.27 mmol) dropwise by syringe. After 2 h, a solution of cyclohexanone (1.03 mL, 9.92 mmol) in 8 mL THF was added dropwise by cannulation at −78° C. After 1 h TLC in 4/1 hexanes/EtOAc shows a major spot at rf=0.4. The reaction was poured into 50 mL saturated NH₄Cl and then the solution was extracted 3×50 mL with EtOAc. The combined organics were dried (magnesium sulfate), filtered and concentrated. The crude product was loaded onto a Biotage 40M column with hexanes and eluted with 4/96 EtOAc/hexanes. Product containing fractions were pooled and concentrated to a clear oil which solidified upon storage in the freezer overnight (1.3 g, 63%); R_{f}=0.2 (9:1 hexanes:EtOAc); ¹H NMR (400 MHz, CDCl₃); δ 7.31 (s, 1H), 7.01 (d, J=10.5 Hz, 1H), 6.95 (d, J=10.4 Hz, 1H), 1.86-1.56 (m, 10H), 1.31 (s, 9H).

1-(1-Azido-cyclohexyl)-3-tert-butyl-5-fluoro-benzene: To a stirred solution of the tertiary alcohol (1.3 g, 5.2 mmol) in 11 mL CH₂Cl₂ at 0° C. under nitrogen was added sodium azide (1.01 g, 15.6 mmol) as a solid. A solution of TFA (1.2 mL, 15.6 mmol) in 5 mL CH₂Cl₂ was then added dropwise by syringe. Immediately a solid began to precipitate. The cooling bath was removed and after 1 h, TLC in 9/1 hexanes/EtOAc showed near complete consumption of starting material. The reaction was allowed to proceed overnight. The reaction was partitioned between CH₂Cl₂ (50 mL) and H₂O (50 mL) and the organics washed 2×50 mL with 3 N NH₄OH and 1×50 mL with brine. The organics were dried (magnesium sulfate), filtered and concentrated to a yellow oil. The material was taken crude into the Staudinger Reduction below.

1-(3-tert-Butyl-5-fluoro-phenyl)-cyclohexylamine hydrochloride salt: To a stirred solution of the azide (800 mg, 2.9 mmol) in 9 mL 95% EtOH at room temperature was added Pearlman's Catalyst. The suspension was put through a vacuum/purge cycle three times with hydrogen gas and then held under 1 atm hydrogen. After 2 h the reaction appeared to be complete by TLC in 9/1 EtOAc/MeOH. The suspension was filtered through GF/F filter paper with 95% EtOH and the filtrate concentrated to a crude oil. The oil was loaded onto a Biotage 40M cartridge with EtOAc and eluted on the Horizon system with a gradient of EtOAc to 10% MeOH in EtOAc. Product containing fractions were pooled and concentrated to a clear oil (540 mg, 75%). The free base was dissolved in 5 mL Et₂O and cooled to 0° C. and treated with 1 M HCl in Et₂O (2 equiv.). A white precipitate formed that was filtered off with hexane, rinsed and dried under high vacuum: retention time (min)=2.73, method [8]; ¹H NMR (400 MHz, DMSO-d₆) δ 8.44 (s, 2H), 7.49 (s, 1H), 7.28-7.20 (m, 2H), 2.32-2.20 (m, 2H), 1.99-1.87 (m, 2H), 1.79-1.65 (m, 2H), 1.50-1.27 (m, 4H), 1.30 (s, 9H); MS (ESI) 249.8.

### EXAMPLE 122

### PREPARATION OF (1S, 2R)-N-[3-[1-(3-TERT-BUTYL-5-FLUORO-PHENYL)-CYCLOHEXYLAMINO]-1-(3,5-DIFLUORO-BENZYL)-2-HYDROXY-PROPYL]-ACETAMIDE

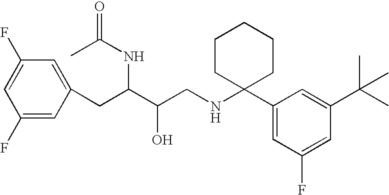

The title compound was synthesized from 1-(3-tert-Butyl-5-fluoro-phenyl)-cyclohexylamine using methods described in EXAMPLE 22: retention time (min)=2.06, method [1]; ¹H NMR (300 MHz, MeOD-d₄); δ 7.48 (d, J=1.3 Hz, 1H), 7.23 (tt, J=12.5, 1.9 Hz, 2H), 6.85-6.70 (m, 3H), 3.95-3.80 (m, 1H), 3.65-3.50 (m, 1H), 3.20 (dd, J=14.2, 3.1 Hz, 1H), 2.75-2.55 (m, 3H), 2.53 (dd, J=14.2, 11.1 Hz, 1H), 2.20-1.70 (m, 5H), 1.83 (s, 3H), 1.70-1.55 (m, 1H), 1.50-1.20 (m, 3H), 1.35 (s, 9H); ¹³C NMR (75 MHz, MeOD-d₄) δ 174.3, 164.8 (d, J=244.9 Hz, 1C), 164.4 (dd, J=246.9, 13.1 Hz, 2C), 162.0 (d, J=13.1 Hz, 1C), 157.0 (d, J=6.7 Hz, 1C), 144.2 (t, J=9.3 Hz, 1C), 138.1 (d, J=7.0 Hz, 1C), 122.0, 114.6 (d, J=21.9 Hz, 1C), 113.4 (d, J=23.5 Hz, 1C), 112.8 (dd, J=17.1, 7.7 Hz, 2C), 102.7 (t, J=25.8 Hz, 1C), 70.4, 65.2, 54.6, 46.0, 36.8, 36.2, 35.2, 33.3, 31.5, 26.0, 23.2, 22.3; MS (ESI) 491.2.

### EXAMPLE 123

### PREPARATION OF 4-AMINO-4-(3-TERT-BUTYL-PHENYL)-CYCLOHEXANONE

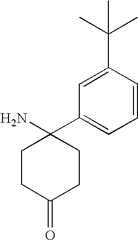

This amine was synthesized from 8-(3-tert-Butyl-phenyl)-1,4-dioxa-spiro[4.5]dec-8-ylamine according to the method described in step 3 of EXAMPLE 26: retention time (min)=1.34, method [4]; MS (ESI) 229.1 (100), 246.1 (40).

### EXAMPLE 124

### PREPARATION OF 1-(3-TERT-BUTYL-PHENYL)-4,4-DIFLUORO-CYCLOHEXYLAMINE

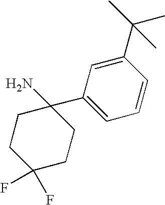

To 4-amino-4-(3-tert-butyl-phenyl)-cyclohexanone (200 mg, 0.82 mmol) was added a solution of bis(2-methoxyethyl)amino-sulfur trifluoride (360 mg, 1.6 mmol) and ethanol (12 μL) in CH₂Cl₂ (1 mL). This was stirred overnight at room temperature. The reaction mixture was quenched with saturated NaHCO₃ (5 mL), and extracted with EtOAc (2×5 mL). The organic extracts were dried (sodium sulfate), filtered and concentrated under reduced pressure. The residue was purified by flash chromatography (10% MeOH/CH₂Cl₂ elution) yielding 20 mg (9%) of material as an oil: R_{f}=0.33 (10% MeOH/CH₂Cl₂); retention time (min)=1.51, method [1]; MS (ESI) 251.1.

### EXAMPLE 125

### PREPARATION OF (1S,2R)-N-[3-[1-(3-TERT-BUTYL-PHENYL)-4,4-DIFLUORO-CYCLOHEXYLAMINO]-1-(3,5-DIFLUORO-BENZYL)-2-HYDROXY-PROPYL]-ACETAMIDE

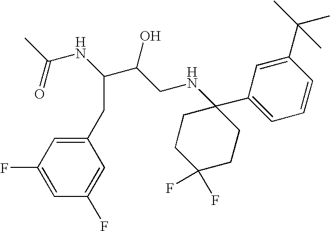

The title compound was synthesized from 1-(3-tert-Butyl-phenyl)-4,4-difluoro-cyclohexylamine according to the method described in EXAMPLE 22. HPLC purification afforded 2.6 mg of white powder as the trifluoroacetic acid salt: retention time (min)=1.85, method [1]; MS (ESI) 509.2.

### EXAMPLE 126

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID BENZYL ESTER FROM 3-AMINO-3-(3-TERT-BUTYL-PHENYL)-PIPERIDINE-1-CARBOXYLIC ACID BENZYL ESTER

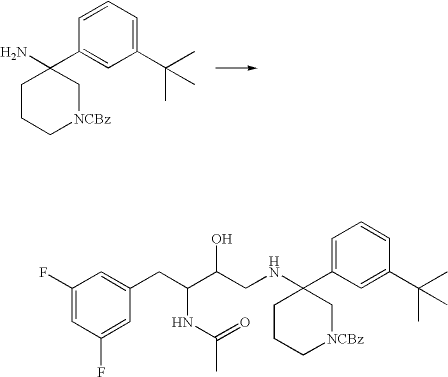

The titled compound was prepared according to the procedure described in EXAMPLE 22 from 3-amino-3-(3-tert-butyl-phenyl)-piperidine-1-carboxylic acid benzyl ester yielding 3-[3-acetylamino-4-(3,5-difluoro-phenyl)-2-hydroxy-butylamino]-3-(3-tert-butyl-phenyl)-piperidine-1-carboxylic acid benzyl ester as a mixture of two diastereomers, which were separated by flash chromatography (50% EtOAc/hexane to 1% MeOH/EtOAc). The mixtures were further purified by HPLC yielding 3-[3-acetylamino-4-(3,5-difluoro-phenyl)-2-hydroxy-butylamino]-3-(3-tert-butyl-phenyl)-piperidine-1-carboxylic acid benzyl ester as a yellow solid. 300 MHz ¹H NMR (CDCl₃); δ 7.62-7.20 (m, 9H), 6.80-6.57 (m, 3H), 6.24 (b s, 1H), 5.82 (b d, J=9 Hz, 1H), 5.22-5.08 (m, 2H), 4.32-4.18 (m, 1H), 4.14-3.71 (m, 5H), 3.62-3.45 (m, 2H), 2.57-1.86 (m, 6H), 1.88 (s, 3H), 1.31 (s, 9H); 75 MHz ¹³C NMR (CDCl₃); δ 172.4, 172.0, 165.0, 164.8, 161.6, 161.5, 161.5, 153.4, 153.2, 136.3, 129.4, 128.4, 128.8, 128.6, 128.5, 128.4, 128,2, 127.0, 124.4, 124.2,123.6, 112.4, 112.1, 102 (t, J=25.1 Hz), 77.4, 68.2, 68.0, 62.5, 62.2, 52.6, 52.2, 43.9, 35.4, 35.2, 31.4, 23.1, 21.0 ppm; Method [1], MS (M+1) =608; retention time (min)=2.20.

### EXAMPLE 127

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID BENZYL ESTER

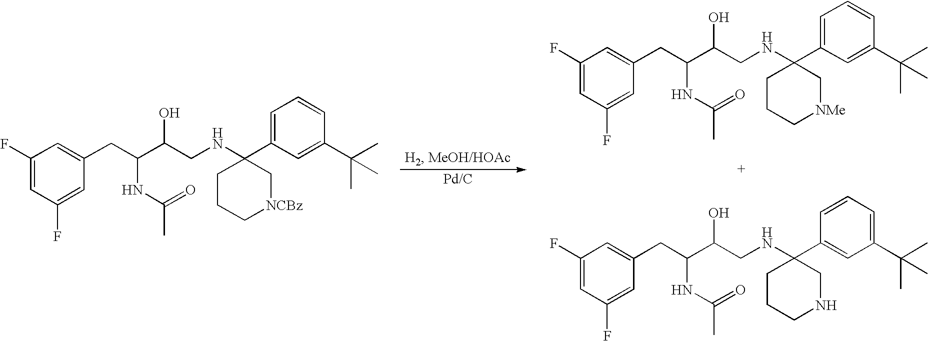

To a stirring of 3-[3-acetylamino-4-(3,5-difluoro-phenyl)-2-hydroxy-butylamino]-3-(3-tert-butyl-phenyl)-piperidine-1-carboxylic acid benzyl ester (400 mg, 0.66 mmol) in MeOH (3 mL) and HOAc (300 μL) was added 10% palladium-carbon (50 mg). The resulting mixture was stirred at room temperature under an atmospheric pressure of hydrogen for 2 days. The mixture was then filtered through a plug of Celite. The Celite plug was washed several times with 10% MeOH/EtOAc. The filtrate was concentrated under reduced pressure yielding a crude mixture, which was subjected to silica gel chromatography. Elution with 100% EtOAc and 2% NH₄OH/10% MeOH/EtOAc afforded N-[3-[3-(3-tert-butyl-phenyl)-piperidin-3-ylamino]-1-(3,5-difluoro-benzyl)-2-hydroxy-propyl]-acetamide (N—H product) and N-[3-[3-(3-tert-butyl-phenyl)-1-methyl-piperidin-3-ylamino]-1-(3,5-difluoro-benzyl)-2-hydroxy-propyl]-acetamide (N-Me product) as an inseparable mixture (3:1). This mixture was then further purified via HPLC.

Analytical data for N-[3-[3-(3-tert-butyl-phenyl)-piperidin-3-ylamino]-1-(3,5-difluoro-benzyl)-2-hydroxy-propyl]-acetamide (N—H product); Method [1], LCMS (M+1)=474; retention time (min)=1.45.

Analytical data for N-[3-[3-(3-tert-butyl-phenyl)-1-methyl-piperidin-3-ylamino]-1-(3,5-difluoro-benzyl)-2-hydroxy-propyl]-acetamide (N-Me product): 300 MHz ¹H NMR (CDCl₃); δ 7.58-7.32 (m, 3H), 7.20-7.14 (m, 1H), 6.83-6.82 (m, 3H), 6.41 (b d, J=17 Hz, 1H), 4.39-4.02 (m, 6H), 3.86-3.77 (m, 1H), 3.75-3.67 (m, 1H), 3.55 (b s, 1H), 3.08-2.92 (m, 1H), 2.88 and 2.86 (s, s, 3H), 2.83-2.73 (m, 1H), 2.66-2.34 (m, 4H), 1.83 and 1.79 (s, s, 3H), 1.31 and 1.30 (s, s, 9H); 75 MHz ¹³C NMR (CDCl₃); δ 164.9, 164.8, 161.5, 153.5, 153.3, 129.5, 127.1, 126.9, 122.9, 122.7, 112.5, 112.1, 102.7, 102.3, 102.2, 77.4, 70.2, 61.0, 60.2, 54.8, 52.8, 52.7, 52.5, 46.7, 46.6, 44.9, 44.9, 36.2, 35.8, 35.3, 35.2, 31.4, 31.4, 23.2, 22.9, 19.4, 19.1 ppm; Method [1], LCMS (M+1)=488; retention time (min)=1.57.

### EXAMPLE 128

### 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID METHYL ESTER FROM 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID BENZYL ESTER

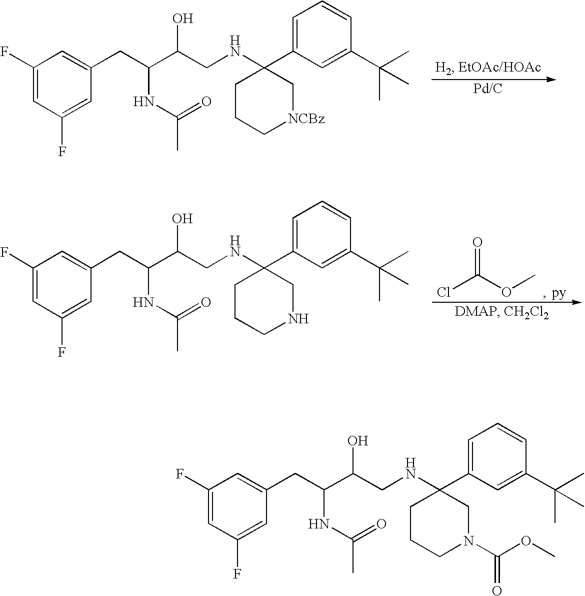

To a stirring solution of 3-[3-acetylamino-4-(3,5-difluoro-phenyl)-2-hydroxy-butylamino]-3-(3-tert-butyl-phenyl)-piperidine-1-carboxylic acid benzyl ester (670 mg, 1.10 mmol) in EtOAc (2 mL) and HOAc (2 mL) was added 10% palladium-carbon (50 mg). The resulting mixture was stirred at room temperature under an atmospheric pressure of hydrogen for 2 days. The mixture was then filtered through a plug of Celite. The Celite plug was washed several times with 10% MeOH/EtOAc. The filtrate was concentrated under reduced pressure yielding a crude mixture, which was subjected to silica gel chromatography. Elution with 100% EtOAc and 2% NH₄OH/10% MeOH/EtOAc afforded the desired amine (400 mg, 77% yield).

To a stirring solution of N-[3-[3-(3-tert-butyl-phenyl)-piperidin-3-ylamino]-1-(3,5-difluoro-benzyl)-2-hydroxy-propyl]-acetamide (48 mg, 0.1 mmol) in CH₂Cl₂ (1 mL) was successively added pyridine, DMAP (2 crystals), and methyl chloroformate (25 mg, 0.2 mmol). The resulting mixture was allowed to react overnight at room temperature. The reaction was quenched with a saturated NaHCO₃ (5 mL) solution and extracted with EtOAc (2×20 mL). The organic layers were washed with brine, dried over sodium sulfate, and filtered. The combined organic layers were evaporated under reduced pressure. The crude mixture was purified via silica gel chromatography. Elution with 50% EtOAc/hexane, 100% EtOAc, and 3% MeOH/EtOAc afforded 3-[3-acetylamino-4-(3,5-difluoro-phenyl)-2-hyd roxy-butylamino]-3-(3-tert-butyl-phenyl)-piperidine-1-carboxylic acid methyl ester (30 mg): 300 MHz ¹H NMR (CDCl₃); δ 7.53-7.21 (m, 4H), 6.76-6.62 (m, 3H), 6.01 (b s, 1H) 4.18-4.02 (m, 2H), 4.01-3.82 (m, 1H), 3.74 (s, 3H), 3.42-3.32 (m, 2H), 3.10-2.93 (m, 2H), 2.72-2.70 (m, 2H), 2.46-2.12 (m, 2H), 2.10-1.76 (m, 4H), 1.91 (s, 3H), 1.33 and 1.32 (s, s, 9H); 75 MHz ¹³C NMR (CDCl₃); δ 164.9, 164.7, 161.6, 161.4, 151.7, 128.4, 124.4, 124.5, 124.3, 123.4, 112.5, 112.3, 112.2, 112.1, 112.0, 102.2 (t, J=23.5 Hz), 77.4, 71.3, 71.0, 56.7, 53.0, 52.8, 44.4, 43.8, 36.2, 35.0, 31.6, 29.9, 29.6, 23.4 ppm; Method [1], LCMS (M+1) 531; retention time (min)=1.86.

### EXAMPLE 129

### PREPARATION OF N-[3-[1-ACETYL-3-(3-TERT-BUTYL-PHENYL)-PIPERIDIN-3-YLAMINO]-1-(3,5-DIFLUORO-BENZYL)-2-HYDROXY-PROPYL]-ACETAMIDE

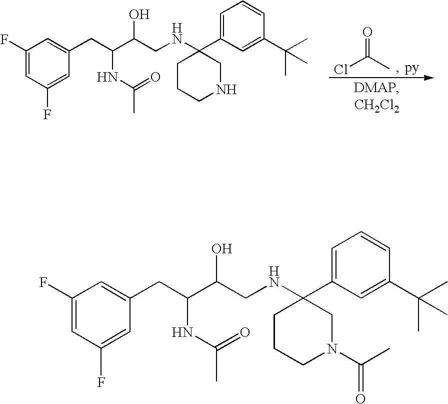

The free amine was converted into N-[3-[1-acetyl-3-(3-tert-butyl-phenyl)-piperidin-3-ylamino]-1-(3,5-difluoro-benzyl)-2-hydroxy-propyl]-acetamide according to the procedure described above. Analytical data for N-[3-[1-acetyl-3-(3-tert-butyl-phenyl)-piperidin-3-ylamino]-1-(3,5-difluoro-benzyl)-2-hydroxy-propyl]-acetamide: 300 MHz ¹H NMR (CDCl₃); δ 7.74-7.24 (m, 4H), 6.81-6.62 (m, 3H), 5.93 (d, J=8.9 Hz, 1H), 4.94 (d, J=13 Hz, 1H), 4.63-4.52 (m, 1H), 4.24-4.08 (m, 1H), 4.05-3.65 (m, 3H), 3.61-3.07 (m, 6H), 3.05-2.85 (m, 2H), 2.80-2.56 (m, 2H), 2.20 and 2.06 (s, s, 3H), 1.90-1.84 (s, s, 3H), 1.32 (s, 9H); 75 MHz ¹³C NMR (CDCl₃); δ 173.1, 172.1, 171.7, 171.5, 165.0, 162.3, 161.8, 161.7, 161.5, 153.6, 153.4, 141.6, 141.0, 135.4, 134.2, 129.5, 129.3, 127.2, 123.8, 123.7, 123.5, 123.0, 112.5, 112.2, 112.2, 102.8, 102.4, 77.5, 70.0, 68.8, 62.8, 62.4, 52.9, 51.7, 47.0, 46.6, 45.6, 44.9, 36.4, 35.3, 35.3, 35.2, 33.9, 31.4, 31.6, 23.0, 22.9, 21.7, 21.1 ppm; Method [1], LCMS (M+1) 516; retention time (min)=1.76.

### EXAMPLE 130

### PREPARATION OF N-[3-[3-(3-TERT-BUTYL-PHENYL)-1-METHANESULFONYL-PIPERIDIN-3-YLAMINO]-1-(3,5-DIFLUORO-BENZYL)-2-HYDROXY-PROPYL]-ACETAMIDE

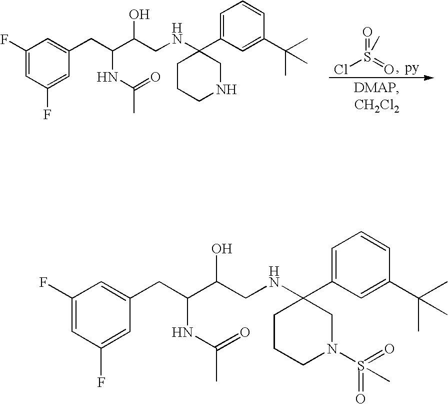

Analytical data of N-[3-[3-(3-tert-butyl-phenyl)-1-methanesulfonyl-piperidin-3-ylamino]-1-(3,5-difluoro-benzyl)-2-hydroxy-propyl]-acetamide: 300 MHz ¹H NMR (CDCl₃); δ 7.61-7.21 (m, 4H), 6.72-6.53 (m, 3H), 5.95 (d, J=10.1 Hz, 1H), 5.69 (d, J=10.1 Hz, 1H), 4.26-4.01 (m, 2H), 3.85-3.60 (m, 4H), 3.48-3.36 (m, 2H), 3.20-3.0 (m, 2H), 2.90-2.72 (m, 3H), 2.85 and 2.80 (s, s, 3H), 2.41-1.82 (m, 6H), 2.18 (s, 3H), 1.95 and 1.87 (s, s, 3H), 1.33 and 1.32 (s, s, 9H); 75 MHz ¹³C NMR (CDCl₃); δ 170.8, 170.7, 164.9, 164.7, 161.6, 161.4, 151.9, 151.8, 142.2, 128.5, 125.0, 124.7, 123.6, 123.5, 123.6, 123.5, 123.5, 112.5, 112.4, 112.3, 112.2, 112.0, 102.6, 102.2, 102.2, 101.9, 77.4, 71.5, 71.0, 56.7, 54.7, 54.3, 53.1, 52.8, 46.5, 43.91, 43.4, 36.5, 36.2, 35.5, 35.1, 34.7, 33.9, 33.3, 31.8, 31.6, 23.5, 23.4, 22.9, 21.3 ppm; Method [1], LCMS (M+1) 552; retention time (min)=1.82 (min).

### EXAMPLE 131

### PREPARATION OF N-[3-[3-(3-TERT-BUTYL-PHENYL)-1-(3-PHENYL-PROPIONYL)-PIPERIDIN-3-YLAMINO]-1-(3,5-DIFLUORO-BENZYL)-2-HYDROXY-PROPYL]-ACETAMIDE

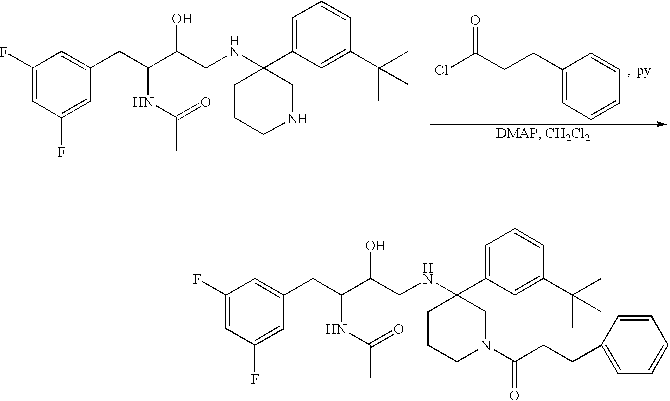

Analytical data for N-[3-[3-(3-tert-butyl-phenyl)-1-(3-phenyl-propionyl)-piperidin-3-ylamino]-1-(3,5-difluoro-benzyl)-2-hydroxy-propyl]-acetamide: 300 MHz ¹H NMR (CDCl₃); δ 7.61-7.22 (m, 9H), 6.72-6.64 (m, 3H), 5.85-5.68 (m, 1H), 4.67-4.41 (m, 1H), 4.24-4.05 (m, 1H), 3.81-3.60 (m, 1H), 3.51-3.21 (m, 2H), 3.20-3.08 (m, 1H), 2.93-2.62 (m, 8H), 2.61-2.09 (m, 5H), 1.94 and 1.90 (s, s, 3H), 1.34 and 1.33 (s, s, 9H); Method [1], LCMS (M+1)=606; retention time (min)=2.1.

### EXAMPLE 132

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID AMIDE

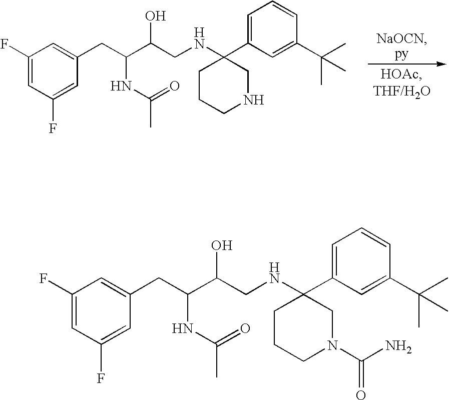

To a stirring solution of the amine (35 mg, 0.074 mmol) in THF/H₂O (0.6 mL each) was added pyridine, acetic acid (2 drops each) and NaOCN (240 mg, 3.7 mmol). The resulting mixture was allowed to react for 24 h. The mixture was then quenched with CH₂Cl₂ (10 mL) and saturated NaHCO₃ solution (10 mL). The layers were separated and the aqueous layer was extracted with EtOAc (2×10 mL). The layers were dried over Na₂SO₄, filtered, and concentrated under reduced pressure. The crude mixture was purified via a silica gel chromatography. Elution of 100% EtOAc, 3% MeOH/EtOAc, and 10% MeOH/EtOAc afforded 3-[3-acetylamino-4-(3,5-difluoro-phenyl)-2-hydroxy-butylamino]-3-(3-tert-butyl-phenyl)-piperidine-1-carboxylic acid amide. This product was further purified using HPLC. Analytical data for the urea compound: 300 MHz ¹H NMR (CDCl₃); δ 7.74-7.32 (m, 4H), 6.62-6.57 (m, 3H), 4.60 (d, J=15.1 Hz, 1H), 4.41-4.31 (m, 1H), 4.20-3.61 (m, 4H), 3.30-2.44 (m, 6H), 2.25-1.96 (m, 2H), 1.83 and 1.76 (s, s, 3H), 1.33 and 1.31 (s, s, 9H); 75 MHz ¹³ C NMR (CDCl₃); δ 171.6, 172.3, 164.8, 164.7, 162.8, 162.3, 161.5, 161.4, 160.2, 160.0, 153.6, 153.5, 141.8, 141.2, 134.5, 134.2, 129.6, 129.5, 127.6, 127.4, 123.6, 123.5, 123.44, 123.4, 123.4, 112.6, 112.4, 112.3, 112.2, 112.1, 102.4, 102.3, 102.2, 77.5, 69.3, 68.7, 62.6, 62.4, 52.5, 51.5, 47.2, 46.7, 43.7, 43.5, 36.1, 35.6, 35.3, 35.3, 31.3, 31.3, 30.1, 29.2, 23.0, 22.9 19.7 ppm; Method [1], LCMS (M+1) 517; retention time(min)=1.69.

The mixture of diastereomers were further purified by HPLC:

Isomer 1: Method [1], LCMS (M+1)=517; retention time (min)=1.673

Isomer 2: Method [1], LCMS (M+1)=517; retention time (min)=1.666

### EXAMPLE 133

### PREPARATION OF N-[3-[3-(3-TERT-BUTYL-PHENYL)-1-HYDROXY-PIPERIDIN-3-YLAMINO]-1-(3,5-DIFLUORO-BENZYL)-2-HYDROXY-PROPYL]-ACETAMIDE

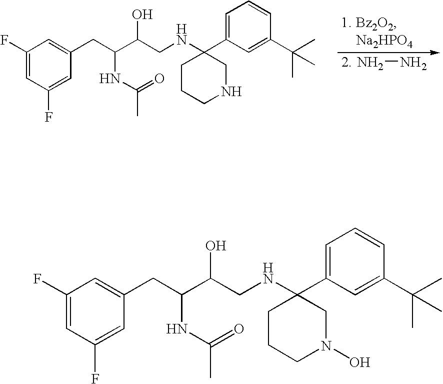

To a stirring mixture of the amine (75 mg, 0.158 mmol) and Na₂HPO4 (114 mg, 0.80 mmol) in THF (1 mL) was added dibenzoylperoxide (45 mg, 0.182 mmol) in THF (0.2 mL) dropwise. After 15 h of stirring, the resulting mixture was then filtered and the solid was washed with 50 mL of CH₂Cl₂. The organic layer was then concentrated under reduced pressure. The insoluble material was then dissolved in 10% NaHCO₃ and CH₂Cl₂ (20 mL, each). The layers were separated and the aqueous layer was extracted with CH₂Cl₂. The combined organic layers were dried over sodium sulfate, filtered, and concentrated under reduced pressure. This crude mixture was directly taken to the next reaction without any further purification. Method [1], LCMS (M+1)=594; retention time (min)=2.24.

To a stirring solution of N-OBz in THF (1 mL) was added hydrazine (200 μL) dropwise at room temperature. After 15 h of stirring, the mixture was then concentrated under reduced pressure. The crude mixture was purified via silica chromatography. Elution with 50% EtOAc/hex, 80% EtOAc/hex, 100% EtOAc, and 5% MeOH/EtOAc afforded N-[3-[3-(3-tert-butyl-phenyl)-1-hydroxy-piperidin-3-ylam ino]-1-(3,5-difluoro-benzyl)-2-hydroxy-propyl]-acetamide which was further purified via HPLC. Analytical data of the hydroxy amine: 300 MHz ¹H NMR (CDCl₃); δ 7.61-7.22 (m, 4H), 6.78-6.61 (m, 3H), 4.18-3.88 (m, 2H), 3.82-3.65 (m, 1H), 3.54 (b s, 1H), 3.21-2.79 (m, 2H), 2.76-2.46 (m, 5H), 2.19 (b s, 1H), 2.14-1.86 (m, 4H), 1.85 (s, 3H), 1.33 and 1.32 (s, s, 9H); Method [1], LCMS (M+1) =490; retention time (min)=1.77.

### EXAMPLE 134

### PREPARATION OF N-[3-[3-(3-TERT-BUTYL-PHENYL)-1-(PIPERIDINE-1-CARBONYL)-PIPERIDIN-3-YLAMINO]-1-(3,5-DIFLUORO-BENZYL)-2-HYDROXY-PROPYL]-ACETAMIDE

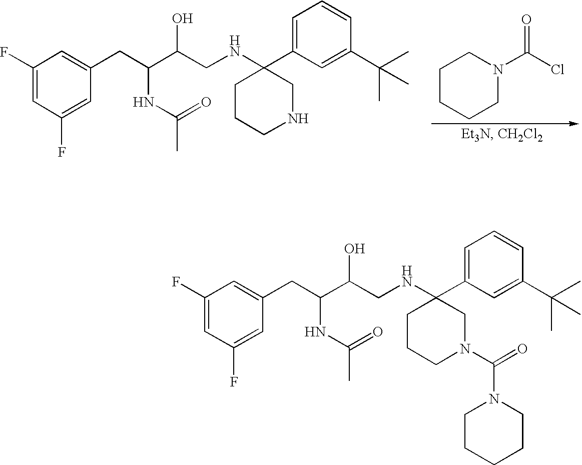

To a stirring solution of the amine (35 mg, 0.74 mmol) in CH₂Cl₂ (1 mL) was added Et₃N and 1-piperidinecarbonyl chloride (20 mg, 1.4 mmol). The resulting mixture was allowed to react at room temperature overnight. The reaction mixture was then quenched with a saturated NaHCO₃ solution. The layers were separated and the aqueous layer was extracted with CH₂Cl₂ (2×10 mL). The combined organic layers were dried over sodium sulfate, filtered, and concentrated under reduced pressure. The crude mixture was purified via silica gel chromatography and then further purified via HPLC. Analytical data for N-[3-[3-(3-tert-butyl-phenyl)-1-(piperidine-1-carbonyl)-piperidin-3-ylamino]-1-(3,5-difluoro-benzyl)-2-hydroxy-propyl]-acetamide: 300 MHz ¹H NMR (CDCl₃); δ [symbol] 7.82 (b s, 1H), 7.66-7.23 (m, 4H), 6.67-6.63 (m, 3H), 4.25 (b d, J=14 Hz, ¹H), 4.0-3.82 (m, 2H), 3.62-3.42 (m, 2H), 3.40-2.96 (m, 9H), 2.93-2.54 (m, 4H), 2.32-1.94 (m, 2H), 1.87 and 1.79 (s, s, 3H), 1.74-1.54 (m, 6H), 1.32 and 1.32 (s, s, 9H); 75 MHz ¹³C NMR (CDCl₃); δ [symbol] 171.8, 164.9, 164.8, 153.2, 141.8, 135.0, 134.7, 129.4, 127.0, 126.9, 123.8, 123.5, 112.5, 112.2, 102.3, 69.2, 62.1, 61.8, 53.2, 52.6, 51.1, 50.3, 48.5, 46.2, 38.6, 38.4, 35.2, 31.4, 23.0, 22.9, 21.2 ppm; Method [1], LCMS (M+1)=585; retention time (min)=2.03.

### EXAMPLE 135

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID DIMETHYLAMIDE

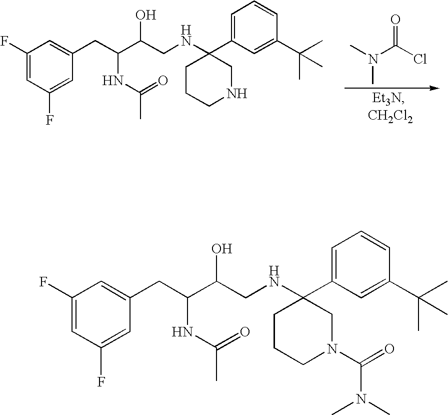

3-[3-acetylamino-4-(3,5-difluoro-phenyl)-2-hydroxy-butylamino]-3-(3-tert-butyl-phenyl)-piperidine-1-carboxylic acid dimethylamide was synthesized analogous to the preparation of N-[3-[3-(3-tert-butyl-phenyl)-1-(piperidine-1-carbonyl)-piperidin-3-ylamino]-1-(3,5-difluoro-benzyl)-2-hydroxy-propyl]-acetamide described above: 300 MHz ¹H NMR (CDCl₃); δ [symbol] 7.67-7.24 (m, 4H), 7.00 (b d, J=8.1 Hz, 1H), 6.70-6.54 (m, 3H), 4.32-4.04 (m, 3H), 3.92-3.65 (m, 2H), 3.62-3.01 (m, 6H), 2.89 and 2.86 (s, s, 6H), 2.78-2.50 (m, 3H), 2.38-1.85 (m, 2H), 1.85 and 1.80 (s, s, 3H), 1.33 and 1.32 (s, s, 9H); Method [1], LCMS (M+1) 545; retention time (min)=1.81.

### EXAMPLE 136

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID

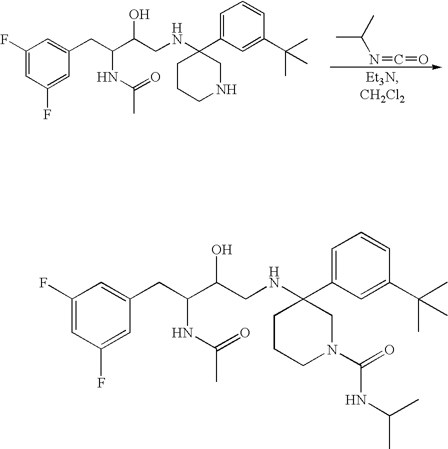

Analytical data for 3-[3-acetylamino-4-(3,5-difluoro-phenyl)-2-hydroxy-butylamino]-3-(3-tert-butyl-phenyl)-piperidine-1-carboxylic acid isopropylamide: 300 MHz ¹H NMR (CDCl₃); δ [symbol] 7.71 (b s, 1H), 7.49-7.29 (m, 4H), 6.73-6.65 (m, 3H), 6.14 (b s, J=8.2 Hz, 1H), 5.01-4.93 (b s, 1H), 4.72-4.67 (m, 1H), 4.18-4.14 (m, 2H), 3.78-3.69 (m, 3H), 3.41-3.35 (m,1H), 3.12-2.78 (m, 3H), 2.66- 2.39 (m, 5H), 2.08-2.05 (m, 1H), 1.87 (s, 3H), 1.34 (s, 9H), 0.90 (t, J=3.6 Hz, 6H); Method [1], LCMS (M+1)=559; retention time (min)=1.93.

### EXAMPLE 137

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID METHYLAMIDE

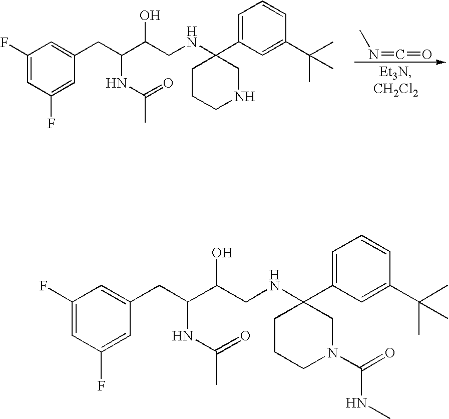

Analytical data for 3-[3-acetylamino-4-(3,5-difluoro-phenyl)-2-hydroxy-butylamino]-3-(3-tert-butyl-phenyl)-piperidine-1-carboxylic acid methylamide: 300 MHz ¹H NMR (CDCl₃); δ [symbol] 7.79 (b s, 1H), 7.56-7.24 (m, 4H), 6.70-6.54 (m, 3H), 6.23-6.21 (m, 1H), 5.81-5.76 (m, 1H), 4.73-4.62 (m, 1H), 4.41-4.32 (m, 1H), 4.23-3.95 (m, 3H), 3.81-3.52 (m, 2H), 3.36-2.96 (m, 4H), 2.86-2.56 (m, 3H), 2.72 (s, 3H), 1.89 and 1.86 (s, s, 3H), 1.34 and 1.33 (s, s, 9H); Method [1], LCMS (M+1) =531; retent time (min)=1.77.

### EXAMPLE 138

### PREPARATION OF 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID BENZYLAMIDE FROM 3-[3-ACETYLAMINO-4-(3,5-DIFLUORO-PHENYL)-2-HYDROXY-BUTYLAMINO]-3-(3-TERT-BUTYL-PHENYL)-PIPERIDINE-1-CARBOXYLIC ACID AMIDE

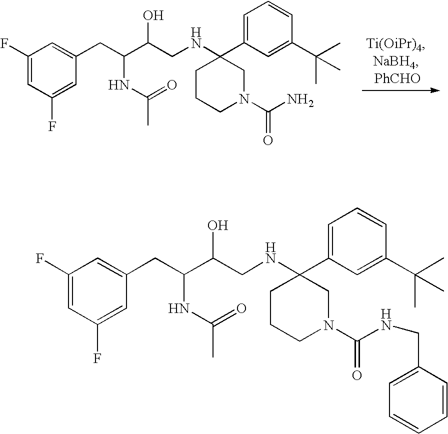

To a stirring solution of 3-[3-acetylamino-4-(3,5-difluoro-phenyl)-2-hydroxy-butylamino]-3-(3-tert-butyl-phenyl)-piperidine-1-carboxylic acid amide (68 mg, 0.14 mmol) in THF (1 mL) at 0° C. was added Ti(OⁱPr)₄ (135 mg, 48 mmol), followed by the addition of benzaldehyde (22 mg, 0.2 mmol) and NaBH₄ (4 mg). The reaction was then allowed to warm to room temperature overnight. After 48 h, the reaction mixture was quenched with a saturated NH₄Cl solution (5 mL). The reaction mixture was then diluted with CH₂Cl₂ (10 mL). The layers were separated and the aqueous layer was extracted with CH₂Cl₂ (2×10 mL). The combined organic layers were washed with brine, dried over sodium sulfate, filtered, and concentrated under reduced pressure yielding crude product. This crude mixture was then purified via silica gel chromatography (100% EtOAc to 15% MeOH/EtOAc) and was then further purified by HPLC. Analytical data for 3-[3-acetylamino-4-(3,5-difluoro-phenyl)-2-hydroxy-butylamino]-3-(3-tert-butyl-phenyl)-piperidine-1-carboxylic acid benzylamide: 300 MHz ¹H NMR (CD₃OD); δ 7.82-7.72 (m, 1H), 7.74-7.17 (m, 8H), 6.87-6.82 (m, 3H), 4.94-4.92 (m, 2H), 4.53-4.15 (m, 3H), 4.12-3.82 (m, 2H), 3.72-3.54 (m, 2H), 3.42-3.24 (m, 4H), 3.18-3.04 (m, 1H), 2.92-2.66 (m, 1H), 2.75-2.21 (m, 3H), 1.86 and 1.80 (s, s, 3H), 1.37 and 1.34 (s, s, 9H); 75 MHz ¹³C NMR (CD₃OD); δ 173.4, 173.0, 164.6, 164.4, 161.3, 161.2, 158.6, 158.4, 152.4, 152.4, 142.9, 142.8, 142.7, 142.5, 139.5, 134.6, 133.9, 128.7, 127.9, 127.7, 126.8, 126.6, 126.5, 126.4, 126.2, 123.9, 123.6, 123.2, 111.5, 111.4, 111.3, 111.3, 111.2, 111.0, 101.5, 101.2 (t, J=16 Hz, 1C), 100.9, 68.7, 68.6, 62.5, 62.1, 52.5, 44.0, 43.1, 35.5, 34.4, 34.1, 30.2, 30.0, 20.8, 20.7, 20.3, 20.0 ppm; Method [1], LCMS (M+1) =607; retention time (min)=2.07.

### EXAMPLE 139

### REPRESENTATIVE PROCEDURE FOR 4-HETEROARYL COMPOUNDS MADE VIA REDUCTIVE AMINATION:

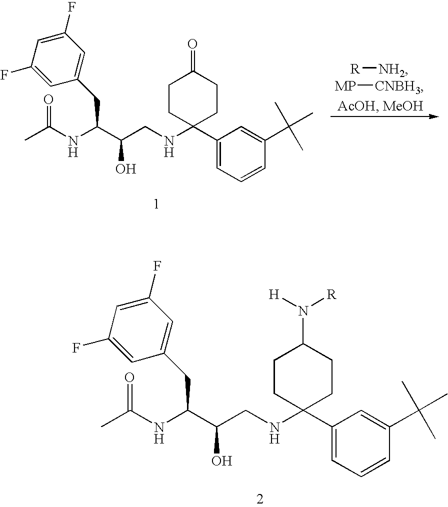

To 97 mgs (0.2 mmol) of N-[3-[1-(3-tert-Butyl-phenyl)-4-oxo-cyclohexylamino]-1-(3,5-difluoro-benzyl)-2-hydroxy-propyl]-acetamide (1) in 1.5 mL of methanol is added 0.24 mmol of heteroaryl amine. The mixture is stirred for 15 min at room temperature. 0.15 mL of glacial acetic acid is then added to the reaction mixture. The mixture is stirred for an additional 30 min. Then, 2.5 equivalents (233 mg) of Argonaut MP-Cyanoborohydride is added to the reaction vial. Each reaction vial is placed on a J-Kem Orbit Shaker block. The reaction temperature is raised to 60° C. The reaction mixture is stirred for 60 h. The resins are filtered out of the reaction mixture. The reaction mixture is then concentrated and isolated via preparative HPLC utilizing a Varian ProStar Preparative HPLC system to leave compounds with general structure 2. LC/MS analysis is conducted utilizing method [1].

N-[3-[1-(3-tert-Butyl-phenyl)-4-(thiazol-2-ylamino)-cyclohexylamino]-1-(3,5-difluoro-benzyl)-2-hydroxy-propyl]-acetamide. ¹H NMR (300 MHz, CD₃OD); δ 7.79-7.64 (m, 1H), 7.64-7.42 (m, 3H), 7.35-7.14 (m, 1H), 6.94-6.65 (m, 3H), 4.14-3.44 (m, 3H), 3.28-3.05 (m, 1H), 3.00-2.79 (m, 2H), 2.77-2.61 (m, 2H), 2.59-2.40 (m, 2H), 2.40-2.22 (m, 1H), 2.20-1.91 (m, 3H), 1.87 (s, 3H), 1.76 (s, 3H). HPLC ret. time 1.425. MS 571.2 (MH+).

N-[3-[1-(3-tert-Butyl-phenyl)-4-(pyridin-2-ylamino)-cyclohexylamino]-1-(3,5-difluoro-benzyl)-2-hydroxy-propyl]-acetamide. ¹H NMR (300 MHz, CD₃OD); δ 8.07-7.80 (m, 1H), 7.79-7.64 (m, 1H), 7.64-7.42 (m, 3H), 7.35-7.14 (m, 1H), 6.94-6.65 (m, 3H), 4.03-3.69 (m, 2H), 3.68-3.43 (m, 1H), 3.28-3.05 (m, 1H), 3.00-2.79 (m, 2H), 2.77-2.61(m, 2H), 2.59-2.40 (m, 2H), 2.40-2.22 (m, 1H), 2.20-2.08 (m, 1H), 2.05-1.90 (m, 2H), 1.87 (s, 3H), 1.76 (s, 3H). HPLC ret. time 1.443. MS 565.2 (MH+).

N-[3-[1-(3-tert-Butyl-phenyl)-4-(pyrimidin-2-ylamino)-cyclohexylamino]-1-(3,5-difluoro-benzyl)-2-hydroxy-propyl]-acetamide. ¹H NMR (300 MHz, CD₃OD); δ 8.46-8.28 (m, 1H), 7.72 (s, 1H), 7.64-7.42 (m, 3H), 6.94-6.65 (m, 3H), 4.11-3.92 (m, 1H), 3.90-3.74 (m, 1H), 3.65 (s, 1H), 3.60-3.46 (m, 1H), 3.28-3.05 (m, 1H), 3.00-2.79 (m, 2H), 2.77-2.61 (m, 2H), 2.59-2.40 (m, 2H), 2.40-2.22 (m, 1H), 2.20-1.91 (m, 3H), 1.87 (s, 3H), 1.76 (s, 3H). HPLC ret. time 1.536. MS 566.2 (MH+).

N-[3-[1-(3-tert-Butyl-phenyl)-4-(1H-pyrazol-3-ylamino)-cyclohexylamino]-1-(3,5-difluoro-benzyl)-2-hydroxy-propyl]-acetamide. ¹H NMR (300 MHz, CD₃OD); δ 7.72 (s, 1H), 7.64-7.42 (m, 3H), 6.94-6.65 (m, 3H), 5.83 (s, 1H), 5.73, (s, 1H), 4.02-3.72 (m, 1H), 3.69-3.42 (m, 1H), 3.28-3.05 (m, 1H), 3.00-2.79 (m, 2H), 2.77-2.61 (m, 2H), 2.59-2.40 (m, 2H), 2.40-2.22 (m, 1H), 2.20-1.91 (m, 1H), 1.87 (s, 3H), 1.76 (s, 3H). HPLC ret. time 1.432. MS 554.2 (MH+).

N-[3-[1-(3-tert-Butyl-phenyl)-4-(pyrazin-2-ylamino)-cyclohexylamino]-1-(3,5-difluoro-benzyl)-2-hydroxy-propyl]-acetamide. ¹H NMR (300 MHz, CD₃OD); δ 7.95 (s, 1H), 7.67 (s, 1H), 7.64-7.42 (m, 3H), 6.94-6.65 (m, 3H), 4.03-3.87 (m, 1H), 3.87-3.75 (m, 1H), 3.65 (s, 1H), 3.60-3.46 (m, 1H), 3.28-3.05 (m, 1H), 2.77-2.61 (m, 2H), 2.59-2.40 (m, 2H), 2.40-2.22 (m, 1H), 2.06-1.93 (m, 1H), 1.87 (s, 3H), 1.76 (s, 3H), HPLC ret. time 1.547. MS 566.2 (MH+).

N-[3-[1-(3-tert-Butyl-phenyl)-4-(pyridin-3-ylamino)-cyclohexylamino]-1-(3,5-difluoro-benzyl)-2-hydroxy-propyl]-acetamide. ¹H NMR (300 MHz, CD₃OD); δ 8.09 (s, 1H), 8.03-7.87 (m, 2H), 7.81-7.66 (m, 3H), 7.60-7.36 (m, 3H), 6.94-6.65 (m, 3H), 4.03-3.78 (m, 1H), 3.69-3.48 (m, 2H), 3.28-3.05 (m, 1H), 3.00-2.79 (m, 2H), 2.77-2.61 (m, 2H), 2.59-2.40 (m, 2H), 2.40-2.22 (m, 1H), 2.22-2.05 (m, 1H), 1.98-1.88 (m, 2H), 1.87 (s, 3H), 1.76 (s, 3H). HPLC ret. time 1.426. MS 565.2 (MH+).

### EXAMPLE 140

### PREPARATION OF HETEROARYL ANALOGS VIA NUCLEOPHILIC DISPLACEMENT

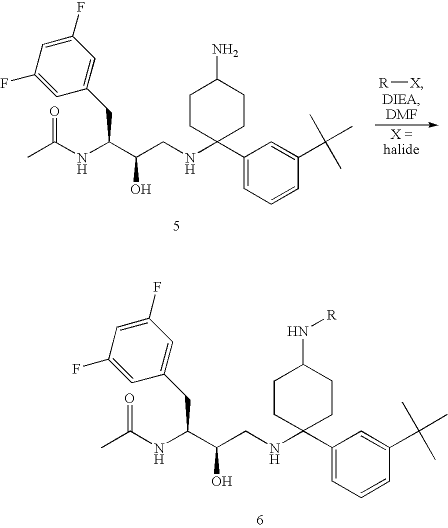

Representative procedure for heteroaryl compounds made via nucleophilic displacement: N-[3-[4-Amino-1-(3-tert-butyl-phenyl)-cyclohexylamino]-1-(3,5-difluoro-benzyl)-2-hydroxy-propyl]-acetamide was synthesized as previously described in EXAMPLE 77.

To 49 mgs (0.1 mmol) of N-[3-[4-Amino-1-(3-tert-butyl-phenyl)-cyclohexylamino]-1-(3,5-difluoro-benzyl)-2-hydroxy-propyl]-acetamide (5) in 1 mL DMF is added 0.15 mmol of heteroaryl halide. 0.1 mL of diisopropylethylamine is added to each reaction vial. Each reaction vial is placed on a J-Kem Orbit Shaker block. The reaction temperature is then raised to 80° C. The reaction mixture is then stirred for 16 h. The reaction mixture is then concentrated and isolated via preparative HPLC utilizing a Varian ProStar Preparative HPLC system to leave compounds with general structure 6. LC/MS analysis is conducted utilizing method [1].

N-[3-[1-(3-tert-Butyl-phenyl)-4-(pyrimidin-2-ylamino)-cyclohexylamino]-1-(3,5-difluoro-benzyl)-2-hydroxy-propyl]-acetamide. ¹H NMR (CD₃OD); δ 8.46-8.28 (m, 1H), 7.72 (s, 1H), 7.64-7.42 (m, 3H), 6.94-6.65 (m, 3H), 4.11-3.92 (m, 1H), 3.90-3.74 (m, 1H), 3.65 (s, 1H), 3.60-3.46 (m, 1H), 3.28-3.05 (m, 1H), 3.00-2.70 (m, 2H), 2.77-2.61 (m, 2H), 2.59-2.40 (m, 2H), 2.40-2.22 (m, 1H), 2.20-1.91 (m, 3H), 1.87 (s, 3H), 1.76 (s, 3H). HPLC ret. time 1.590. MS 566.2 (MH+).

N-[3-[4-(3-Bromo-[1,2,4]thiadiazol-5-ylamino)-1-(3-tert-butyl-phenyl)-cyclohexylamino]-1-(3,5-difluoro-benzyl)-2-hydroxy-propyl]-acetamide. ¹H NMR (CD₃OD); δ 7.72 (s, 1H), 7.64-7.42 (m, 3H), 6.94-6.65 (m, 3H), 4.02-3.89 (m, 1H), 3.87-3.71 (m, 1H), 3.65 (s, 1H), 3.60-3.46 (m, 1H), 3.28-3.05 (m, 1H), 3.00-2.79 (m, 2H), 2.77-2.61 (m, 2H) 2.59-2.40 (m, 2H), 2.40-2.22 (m, 1H), 2.20-1.91, (m, 3H), 1.87 (s, 3H), 1.76 (s, 3H). HPLC ret. time 1.970. MS 651.2 (MH+).

### EXAMPLE 141

### PREPARATION OF CIS/TRANS 4-METHYL-CYCLOHEXANECARBOXYLIC ACID METHYL ESTER

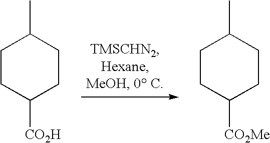

A 2.0 M solution of trimethylsilyidiazomethane in hexanes (11.0 mL, 22.0 mmol) was added to a solution of a mixture of cis/trans isomers of 4-methyl-cyclohexanecarboxylic acid (2.0 mL, 14.1 mmol) in methanol (14 mL) and hexane (14 mL). The clear solution turned yellow following the addition of the trimethylsilyldiazomethane. The solution was concentrated to yield a mixture of cis/trans isomers of 4-methyl-cyclohexanecarboxylic acid methyl ester.

¹H NMR (300 MHz, CDCl₃); δ 3.68 and 3.66 (s, 3 H), 2.51 and 2.21 (m and tt, J=3.6 Hz, and 12.2 Hz, 1H), 1.96 (m, 3 H), 1.74-1.15 (broad m, 6 H), 0.89 (m, 3 H).

### EXAMPLE 142

### PREPARATION OF CIS/TRANS 1-(3-TERT-BUTYL-PHENYL)-4-METHYL-CYCLOHEXANECARBOXYLIC ACID METHYL ESTER

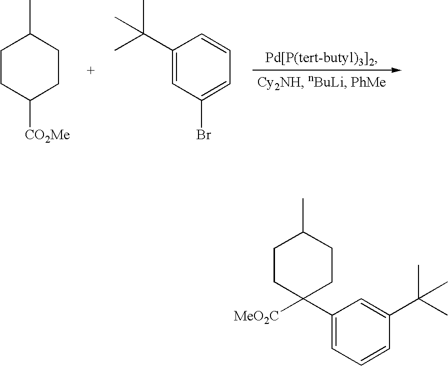

A 1.6 M solution of ⁿbutyllithium (1.7 mL, 2.72 mmol) was added to a solution of dicyclohexylamine (0.52 mL, 2.61 mmol) in toluene (10 mL). After stirring for 5 min, a mixture of cis/trans isomers of 4-methyl-cyclohexanecarboxylic acid methyl ester (342 mg, 2.19 mmol) was added. After stirring for 10 min,1-bromo-3-tert-butyl-benzene (428 mg, 2.01 mmol) and bis(tri-tert-butylphosphine)palladium(0) (52 mg, 102 μmol) was sequentially added. After stirring for 20 h, the solution was diluted with 10% aqueous hydrochloric acid, and extracted with diethyl ether. The combined organic extracts were dried over magnesium sulfate, filtered, and concentrated. The residue was flash chromatographed with 49:1, 24:1, and 23:2 hexanes:ethyl acetate as the eluant to yield 484 mg (84% yield) of a mixture of cis/trans isomers of 1-(3-tert-butyl-phenyl)-4-methyl-cyclohexanecarboxylic acid methyl ester as a light yellow oil.

¹H NMR (300 MHz, CDCl₃); δ 7.51 and 7.40 (t and m, J=1.9 Hz, 1H), 7.33-7.13 (m, 3 H), 3.65 (s, 3 H), 2.62 (m, 2H), 1.77-1.02 (broad m, 7 H), 1.30 (s, 9H), 0.91 (d, J=6.5 Hz, 3 H).

### EXAMPLE 143

### PREPARATION OF CIS/TRANS 1-(3-TERT-BUTYL-PHENYL)-4-METHYL-CYCLOHEXANECARBOXYLIC ACID

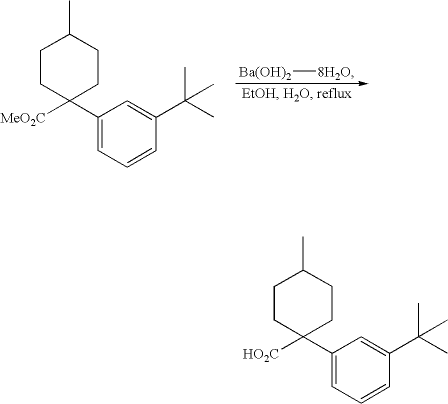

Barium hydroxide-octahydrate (968 mg, 3.07 mmol), and a mixture of cis/trans isomers of 1-(3-tert-butyl-phenyl)-4-methyl-cyclohexanecarboxylic acid methyl ester in ethanol (10 mL) and water (10 mL) was placed into a preheated oil bath at 85° C. After heating at reflux for 18 h, the solution was diluted with 10% aqueous hydrochloric acid, and extracted with methylene chloride. The combined organic extracts were dried over magnesium sulfate, filtered, and concentrated to yield 285 mg (69% yield) of a mixture of cis/trans isomers of 1-(3-tert-butyl-phenyl)-4-methyl-cyclohexanecarboxylic acid as a light yellow oil.

¹H NMR (300 MHz, CDCl₃); δ 7.51 and 7.48 (t and s, J=1.9 Hz, 1H), 7.33-7.14 (m, 3 H), 2.65 (d, J=12.6 Hz, 2H), 1.77-1.10 (broad m, 7 H), 1.31 (s, 9H), 0.92 and 0.88 (both d, both J=6.4 Hz, 3 H).

### EXAMPLE 144

### PREPARATION OF 1-(3-TERT-BUTYL-PHENYL)-4-METHYL-CYCLOHEXYLAMINE

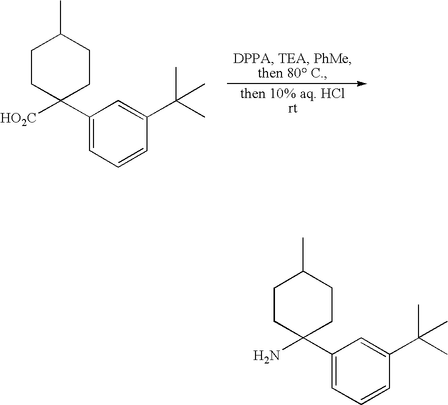

Diphenylphosphoryl azide (0.26 mL, 1.20 mmol) was added to a solution of a mixture of cis/trans 1-(3-tert-butyl-phenyl)-4-methyl-cyclohexanecarboxylic acid (275 mg, 1.00 mmol) and triethylamine (0.19 mL, 1.36 mmol) in toluene (5 mL). After stirring at ambient temperature for 16 h, the solution was placed into a preheated oil bath at 80° C. Bubbling was observed. After stirring for 1 h at 80° C., the bubbling had ceased and the solution was cooled to ambient temperature. Dioxane (2.5 mL) and 10% aqueous hydrochloric acid (2.5 mL) was added and stirred vigorously for 18 h. The aqueous layer was made alkaline with aqueous 3 N NaOH and extracted with methylene chloride. The combined organic extracts were dried over magnesium sulfate, filtered, and concentrated. The residue was flash chromatographed with 19:1:0.1, 9:1:0.1, 17:3:0.3, and 4:1:0.1 methylene chloride:methanol:concentrated ammonium hydroxide as the eluant to yield 75 mg (30% yield) of a single isomer of 1-(3-tert-butyl-phenyl)-4-methyl-cyclohexylamine.

¹H NMR (300 MHz, CDCl₃); δ 7.51 (d, J=1.9 Hz, 1H), 7.37-7.27 (m, 3H), 1.77-1.10 (broad m, 9 H), 1.34 (s, 9H), 0.98 (d, J=5.7 Hz, 3 H). Method [1] Retention time 1.55 min by HPLC and 1.62 min by MS (M-NH₂=229).

### EXAMPLE 145

### [3-[1-(3-TERT-BUTYL-PHENYL)-4-METHYL-CYCLOHEXYLAMINO]-1-(3,5-DIFLUORO-BENZYL)-2-HYDROXY-PROPYL]-CARBAMIC ACID TERT-BUTYL ESTER

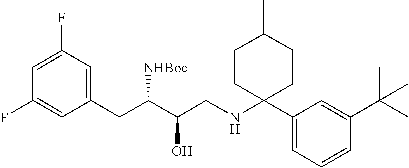

Method [1] Retention time 2.34 min by HPLC and 2.40 min by MS (M+=545).

### EXAMPLE 146

### 3-AMINO-1-[1-(3-TERT-BUTYL-PHENYL)-4-METHYL-CYCLOHEXYLAMINO]-4-(3,5-DIFLUORO-PHENYL)-BUTAN-2-OL

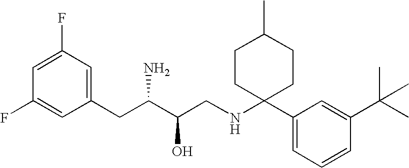

Method [1] Retention time 1.56 min by HPLC and 1.63 min by MS (M+=445).

### EXAMPLE 147

### N-[3-[1-(3-TERT-BUTYL-PHENYL)-4-METHYL-CYCLOHEXYLAMINO]-1-(3,5-DIFLUORO-BENZYL)-2-HYDROXY-PROPYL]-ACETAMIDE

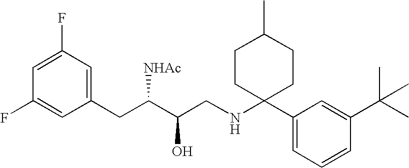

¹H NMR (300 MHz, CDCl₃); δ 9.35 (broad s, 1H), 8.12 (broad s, 1H), 7.66 (s, 1H), 7.43-7.27 (m, 3H), 6.71-6.47 (broad m, 3H), 4.11 (broad s,1H), 3.75 (broad s, 2H), 3.03 (dd, J=4.2 Hz and 14.2 Hz, 1H), 2.75 (dd, J=8.9 Hz, and 14.2 Hz, 1H), 2.55 (m, 4H), 2.08-1.77 (broad m, 5H), 1.85 (s, 3H), 1.63-1.17 (broad m, 2H), 1.32 (s, 9H), 1.04 (d, J=5.7 Hz, 3H). Method [1] Retention time 2.09 min by HPLC and 2.16 min by MS (M+=487).

### EXAMPLE 148

### PREPARATION OF 1-THIOPHEN-3-YL-CYCLOHEXANECARBOXYLIC ACID METHYL ESTER

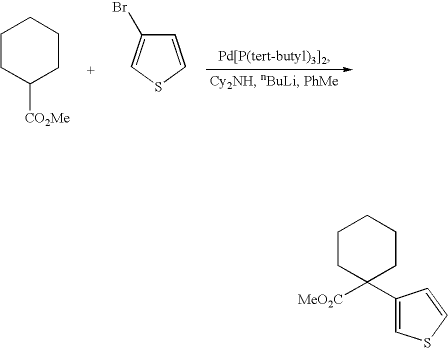

A 1.6 M solution of ⁿbutyllithium (25.0 mL, 40.0 mmol) was added to a solution of dicyclohexylamine (7.8 mL, 39.1 mmol) in toluene (60 mL). After stirring for 5 min, cyclohexanecarboxylic acid methyl ester (4.8 mL, 33.6 mmol) was added. After stirring for 10 min, 1-bromo-thiophene (2.8 mL, 29.6 mmol) and bis(tri-tert-butylphosphine)palladium(0) (312 mg, 610 μmol) was sequentially added. After stirring for 24 h, the solution was diluted with 10% aqueous hydrochloric acid, filtered through a Buchner funnel, and the solid was washed with diethyl ether. The aqueous layer was extracted with diethyl ether, the combined organic extracts were dried over magnesium sulfate, filtered, and concentrated. The residue was flash chromatographed with 99:1, 49:1, and 24:1 hexanes:ethyl acetate as the eluant to yield 4.93 g (74% yield) of 1-thiophen-3-yl-cyclohexanecarboxylic acid methyl ester as a light yellow oil. ¹H NMR (300 MHz, CDCl₃); δ 7.24 (m, 1H), 7.10 (m, 2H), 3.65 (s, 3H), 2.46 (d, J=6.7 Hz 2H), 1.78-1.26 (broad m, 8H).

### EXAMPLE 149

### PREPARATION OF 1-THIOPHEN-3-YL-CYCLOHEXANECARBOXYLIC ACID

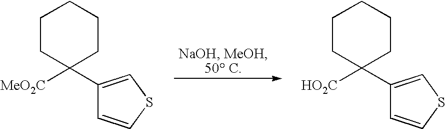

A 3 N solution of aqueous sodium hydroxide (5.0 mL, 15.0 mmol) was added to a solution of 1-thiophen-3-yl-cyclohexanecarboxylic acid methyl ester (500 mg, 2.23 mmol) in methanol (10 mL) and was placed into a preheated oil bath at 50° C. After stirring for 18 h, the solution was concentrated, diluted with 10% aqueous hydrochloric acid, and extracted with methylene chloride. The combined organic extracts were dried over magnesium sulfate, filtered, and concentrated to yield 450 mg (96% yield) of 1-thiophen-3-yl-cyclohexanecarboxylic acid as a white solid. ¹H NMR (300 MHz, CDCl₃); δ 7.24 (m, 1H), 7.10 (m, 2H), 2.46 (d, J=6.7 Hz, 2H), 1.78-1.26 (broad m, 8H).

### EXAMPLE 150

### PREPARATION OF 1-THIOPHEN-3-YL-CYCLOHEXYLAMINE

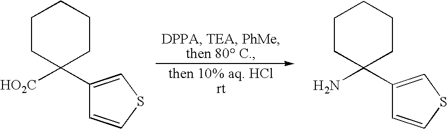

Diphenylphosphoryl azide (1.0 mL, 4.63 mmol) was added to a solution of 1-thiophen-3-yl-cyclohexanecarboxylic acid (450 mg, 2.14 mmol) and triethylamine (1.00 mL, 7.17 mmol) in toluene (10 mL). After stirring at ambient temperature for 16 h, the solution was placed into a preheated oil bath at 80° C. Bubbling was observed. After stirring for 1 h at 80° C., the bubbling had ceased and the solution was cooled to ambient temperature. Dioxane (5 mL) and 10% aqueous hydrochloric acid (5 mL) was added and stirred vigorously for 18 h. The aqueous layer was made alkaline with aqueous 3 N NaOH and extracted with methylene chloride. The combined organic extracts were dried over magnesium sulfate, filtered, and concentrated. The residue was flash chromatographed with 19:1:0.1, 9:1:0.1, 17:3:0.3, and 4:1:0.1 methylene chloride:methanol:concentrated ammonium hydroxide as the eluant to yield 1-thiophen-3-yl-cyclohexylamine as an impure product. Method [1] Retention time 0.43 min by HPLC and 0.50 min by MS (M−NH₂=165).

### EXAMPLE 151

### [1-(3,5-DIFLUORO-BENZYL)-2-HYDROXY-3-(1-THIOPHEN-3-YL-CYCLOHEXYLAMINO)-PROPYL]-CARBAMIC ACID TERT-BUTYL ESTER

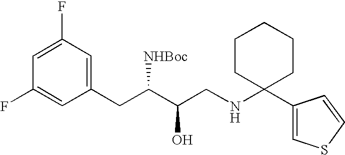

Method [1] Retention time 1.78 min by HPLC and 1.85 min by MS (M+=481).

### EXAMPLE 152

### 3-AMINO-4-(3,5-DIFLUORO-PHENYL)-1-(1-THIOPHEN-3-YL-CYCLOHEXYLAMINO)-BUTAN-2-OL

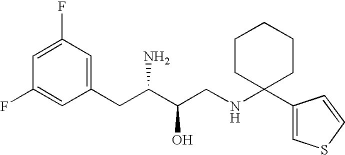

Method [1] Retention time 1.26 min by HPLC and 1.29 min by MS (M+=381).

### EXAMPLE 153

### N[1-(3,5-DIFLUORO-BENZYL)-2-HYDROXY-3-(1-THIOPHEN-3-YL-CYCLOHEXYLAMINO)-PROPYL]-ACETAMIDE

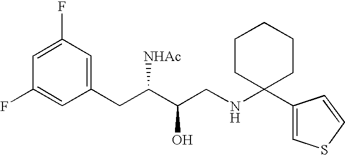

¹H NMR (300 MHz, CDCl₃); δ 9.19 (broad s, 1H), 8.18 (broad s, 1H), 7.42 (m, 2H), 7.21 (dd, J=1.2 Hz and 5.0 Hz, 1H), 6.69 (broad m, 3H), 4.45 (broad s, 2H), 4.00 (broad s, 1H), 3.80 (broad s, 1H), 2.97 (dd, J=4.1 Hz and 14.3 Hz, 1H), 2.66 (m, 2H), 2.48 (m, 3H), 1.99 (m, 2H), 1.83 (s, 3H), 1.75 (broad s, 2H), 1.59 (broad s, 1 H), (broad m, 3H). Method [1] Retention time 1.49 min by HPLC and 1.52 min by MS (M+=423).

### EXAMPLE 154

### PREPARATION OF CIS/TRANS 3-METHYL-CYCLOHEXANECARBOXYLIC ACID 2-TRIMETHYLSILANYL-ETHYL ESTER

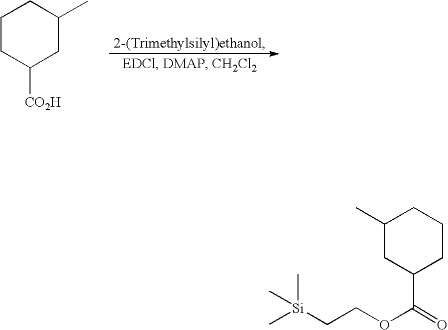

A mixture of cis/trans isomers of 3-methyl-cyclohexanecarboxylic acid (1.44 g, 10.1 mmol), 2-trimethylsilylethanol (1.30 g, 11.0 mmol), 4-dimethylaminopyridine (128 mg, 1.05 mmol), and 1-(3-dimethylaminopropyl)-3-ethylcarbodiimide hydrochloride (2.12 g, 11.1 mmol) in methylene chloride (10 mL) was stirred for 36 h. The solution was diluted with 10% aqueous hydrochloric acid and extracted with methylene chloride. The combined organic extracts were dried over magnesium sulfate, filtered, and concentrated to yield 2.45 g (100% yield) of a mixture of cis/trans isomers of 3-methyl-cyclohexanecarboxylic acid 2-trimethylsilanyl-ethyl ester as a clear oil. ¹H NMR (300 MHz, CDCl₃); δ 4.15 (m, 2H), 2.59 and 2.26 (m and tt, J=3.5 Hz, and 12.1 Hz, 1H), 1.98-1.19 (broad m, 8H), 1.12-0.93 (broad m, 3H), 0.90 (d and d, J=6.5 Hz and 6.7 Hz, 3H), 0.04 (s, 9H).

### EXAMPLE 155

### PREPARATION OF CIS/TRANS 1-(3-TERT-BUTYL-PHENYL)-3-METHYL-CYCLOHEXANECARBOXYLIC ACID 2-TRIMETHYLSILANYL-ETHYL ESTER

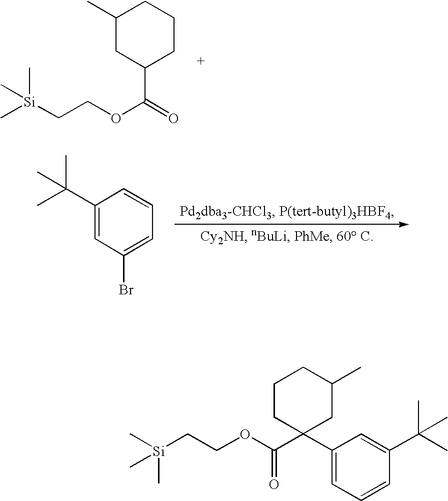

A 1.6 M solution of ⁿbutyllithium (0.85 mL, 1.36 mmol) was added to a solution of dicyclohexylamine (0.27 mL, 1.36 mmol) in toluene (5 mL). After stirring for 5 min, a mixture of cis/trans isomers of 3-methyl-cyclohexanecarboxylic acid 2-trimethylsilanyl-ethyl ester (269 mg, 1.11 mmol) was added. After stirring for 30 min, 1-bromo-3-tert-butyl-benzene (250 mg, 1.17 mmol) was added followed by the simultaneous addition of tri-tert-butylphosphonium tetrafluoroborate (31 mg, 107 μmol) and tris(dibenzylideneacetone)dipalladium(0)-chloroform adduct (54 mg, 52.2 μmol). The solution was placed into a preheated oil bath at 60° C. After stirring for 20 h, the solution was diluted with 10% aqueous hydrochloric acid, and extracted with diethyl ether. The combined organic extracts were dried over magnesium sulfate, filtered, and concentrated. The residue was flash chromatographed with 49:1, 24:1, and 23:2 hexanes:ethyl acetate as the eluant to yield 250 mg (62% yield) of a mixture of cis/trans isomers of 1-(3-tert-butyl-phenyl)-3-methyl-cyclohexanecarboxylic acid 2-trimethylsilanyl-ethyl ester as a yellow oil. Method [2] Retention time 3.64 min by HPLC and 3.68 min by MS (M+Na=397).

### EXAMPLE 156

### PREPARATION OF CIS/TRANS 1-(3-TERT-BUTYL-PHENYL)-3-METHYL-CYCLOHEXANECARBOXYLIC ACID

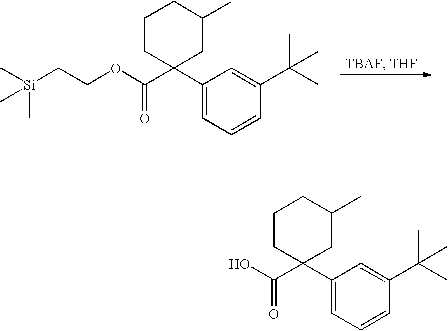

A 1.0 M solution of tetrabutylammonium fluoride in tetrahydrofuran (2.5 mL, 2.5 mmol) was added to a solution of a mixture of cis/trans isomers of 1-(3-tert-butyl-phenyl)-3-methyl-cyclohexanecarboxylic acid 2-trimethylsilanyl-ethyl ester (500 mg, 1.34 mmol) in tetrahydrofuran (10 mL). After stirring for 24 h, the solution was diluted with 10% aqueous hydrochloric acid, and extracted with diethyl ether. The combined organic extracts were dried over magnesium sulfate, filtered, and concentrated to yield 419 mg (impure) of a mixture of cis/trans isomers of 1-(3-tert-butyl-phenyl)-3-methyl-cyclohexanecarboxylic acid as a brown viscous oil.

### EXAMPLE 157

### PREPARATION OF CIS/TRANS 1-(3-TERT-BUTYL-PHENYL)-3-METHYL-CYCLOHEXYLAMINE

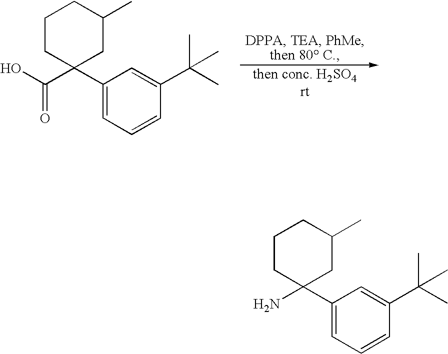

Diphenylphosphoryl azide (0.34 mL, 1.57 mmol) was added to a solution of a mixture of cis/trans isomers of 1-(3-tert-butyl-phenyl)-3-methyl-cyclohexanecarboxylic acid (ca.1.34 mmol) and triethylamine (0.24 mL, 1.72 mmol) in toluene (6 mL). After stirring at ambient temperature for 16 h, the solution was placed into a preheated oil bath at 80° C. Bubbling was observed. After stirring for 1 h at 80° C., the bubbling had ceased and the solution was cooled to ambient temperature. Concentrated sulfuric acid was added and stirred vigorously for 2 min. The aqueous layer was made alkaline with aqueous 3 N NaOH and extracted with methylene chloride. The combined organic extracts were dried over magnesium sulfate, filtered, and concentrated. The residue was flash chromatographed with 99:1:0.1, 49:1:0.1, 24:1:0.1, 23:2:0.2, 22:3:0.3, 21:4:0.4, and 4:1:0.1 methylene chloride/methanol:concentrated ammonium hydroxide as the eluant to yield 185 mg (impure) of a mixture of cis/trans isomers of 1-(3-tert-butyl-phenyl)-3-methyl-cyclohexylamine. Method [1] Retention time 1.75 min by HPLC and 1.82 min by MS (M−NH₂=229).

### EXAMPLE 158

### [3-[1-(3-TERT-BUTYL-PHENYL)-3-METHYL-CYCLOHEXYLAMINO]-1-(3,5-DIFLUORO-BENZYL)-2-HYDROXY-PROPYL]-CARBAMIC ACID TERT-BUTYL ESTER

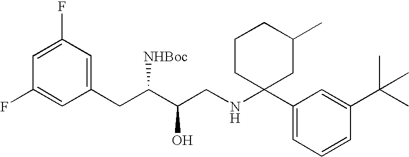

Method [1] Retention time 2.48 min by HPLC and 2.55 min by MS (M+=545).

### EXAMPLE 159

### 3-AMINO-1-[1-(3-TERT-BUTYL-PHENYL)-3-METHYL-CYCLOHEXYLAMINO]-4-(3,5-DIFLUORO-PHENYL)-BUTAN-2-OL

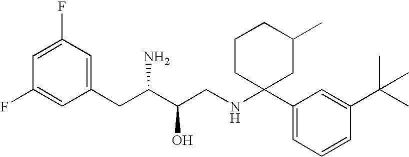

Method [1] Retention time 1.92 min by HPLC and 2.01 min by MS (M+=445).

### EXAMPLE 160

### N-[3-[1-(3-TERT-BUTYL-PHENYL)-3-METHYL-CYCLOHEXYLAMINO]-1-(3,5-DIFLUORO-BENZYL)-2-HYDROXY-PROPYL]-ACETAMIDE

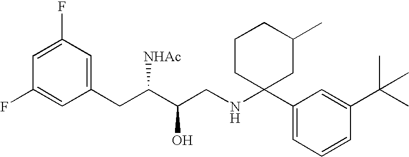

Method [1] Retention time 2.06 min by HPLC and 2.16 min by MS (M+=487).

### EXAMPLE 161

### PREPARATION OF CIS/TRANS 2-METHYL-CYCLOHEXANECARBOXYLIC ACID 2-TRIMETHYLSILANYL-ETHYL ESTER

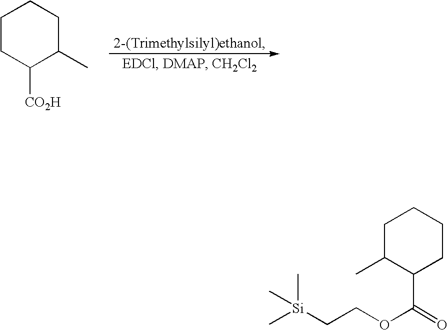

A mixture of cis/trans isomers of 2-methyl-cyclohexanecarboxylic acid (1.44 g, 10.1 mmol), 2-trimethylsilylethanol (1.31 g, 11.1 mmol), 4-dimethylaminopyridine (123 mg, 1.01 mmol), and 1-(3-dimethylaminopropyl)-3-ethylcarbodiimide hydrochloride (2.11 g, 11.0 mmol) in methylene chloride (10 mL) was stirred for 36 h. The solution was diluted with 10% aqueous hydrochloric acid and extracted with methylene chloride. The combined organic extracts were dried over magnesium sulfate, filtered, and concentrated to yield 2.45 g (100% yield) of a mixture of cis/trans isomers of 2-methyl-cyclohexanecarboxylic acid 2-trimethylsilanyl-ethyl ester as a clear oil. ¹H NMR (300 MHz, CDCl₃); δ 4.16 (m, 2H), 2.47 (m, 1H), 2.14 (m, 1H), 1.77-1.20 (broad m, 8H), 0.98 (m, 5H), 0.04 (s, 9H).

### EXAMPLE 162

### PREPARATION OF CIS/TRANS 1-(3-TERT-BUTYL-PHENYL)-2-METHYL-CYCLOHEXANECARBOXYLIC ACID 2-TRIMETHYLSILANYL-ETHYL ESTER

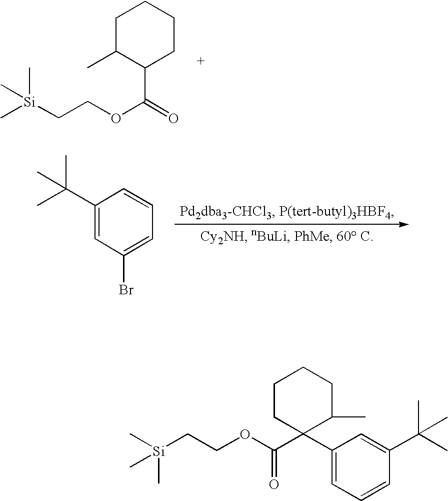

A 1.6 M solution of ⁿbutyllithium (0.85 mL, 1.36 mmol) was added to a solution of dicyclohexylamine (0.27 mL, 1.36 mmol) in toluene (5 mL). After stirring for 5 min, a mixture of cis/trans isomers of 2-methyl-cyclohexanecarboxylic acid 2-trimethylsilanyl-ethyl ester (269 mg, 1.11 mmol) was added. After stirring for 30 min, 1-bromo-3-tert-butyl-benzene (248 mg, 1.16 mmol) was added followed by the simultaneous addition of tri-tert-butylphosphonium tetrafluoroborate (31 mg, 107 μmol) and tris(dibenzylideneacetone)dipalladium(0)-chloroform adduct (51 mg, 49.3 μmol). The solution was placed into a preheated oil bath at 60° C. After stirring for 20 h, the solution was diluted with 10% aqueous hydrochloric acid, and extracted with diethyl ether. The combined organic extracts were dried over magnesium sulfate, filtered, and concentrated. The residue was flash chromatographed with 49:1, 24:1, and 23:2 hexanes:ethyl acetate as the eluant to yield 375 mg (90% yield) of a mixture of cis/trans isomers of 1-(3-tert-butyl-phenyl)-2-methyl-cyclohexanecarboxylic acid 2-trimethylsilanyl-ethyl ester as a yellow oil. Method [2] Retention time 3.67 min by HPLC and 3.75 min by MS (M+Na=397). Method [2] Retention time 3.77 min by HPLC and 3.85 min by MS (M+Na=397).

### EXAMPLE 163

### PREPARATION OF CIS/TRANS 1-(3-TERT-BUTYL-PHENYL)-2-METHYL-CYCLOHEXANECARBOXYLIC ACID

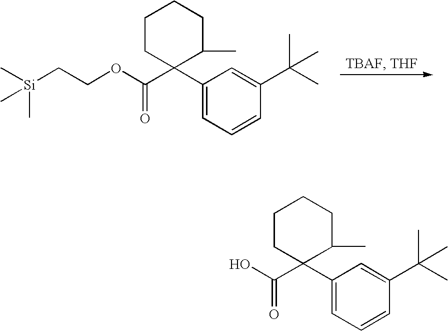

A 1.0 M solution of tetrabutylammonium fluoride in tetrahydrofuran (4.0 mL, 4.00 mmol) was added to a solution of a mixture of cis/trans isomers of 1-(3-tert-butyl-phenyl)-2-methyl-cyclohexanecarboxylic acid 2-trimethylsilanyl-ethyl ester (610 mg, 1.63 mmol) in tetrahydrofuran (10 mL). After stirring for 24 h, the solution was diluted with 10% aqueous hydrochloric acid, and extracted with diethyl ether. The combined organic extracts were dried over magnesium sulfate, filtered, and concentrated to yield 360 mg (80% yield) of a mixture of cis/trans isomers of 1-(3-tert-butyl-phenyl)-2-methyl-cyclohexanecarboxylic acid as a yellow oil.

### EXAMPLE 164

### PREPARATION OF CIS/TRANS 1-(3-TERT-BUTYL-PHENYL)-2-METHYL-CYCLOHEXYLAMINE

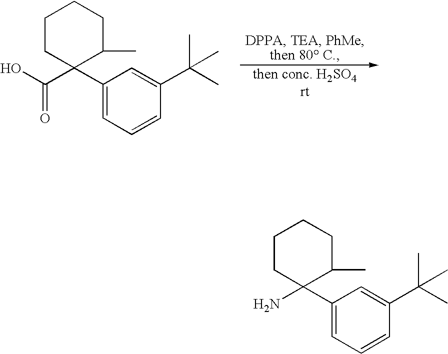

Diphenylphosphoryl azide (0.34 mL, 1.57 mmol) was added to a solution of a mixture of cis/trans isomers of 1-(3-tert-butyl-phenyl)-2-methyl-cyclohexanecarboxylic acid (ca.1.34 mmol) and triethylamine (0.24 mL, 1.72 mmol) in toluene (6 mL). After stirring at ambient temperature for 16 h, the solution was placed into a preheated oil bath at 80° C. Bubbling was observed. After stirring for 1 h at 80° C., the bubbling had ceased and the solution was cooled to ambient temperature. Concentrated sulfuric acid was added and stirred vigorously for 2 min. The aqueous layer was made alkaline with aqueous 3 N NaOH and extracted with methylene chloride. The combined organic extracts were dried over magnesium sulfate, filtered, and concentrated. The residue was flash chromatographed with 99:1:0.1, 49:1:0.1, 24:1:0.1, 23:2:0.2, 22:3:0.3, 21:4:0.4, and 4:1:0.1 methylene chloride/methanol/concentrated ammonium hydroxide as the eluant to yield 95 mg (30% yield) of a mixture of cis/trans isomers of 1-(3-tert-butyl-phenyl)-2-methyl-cyclohexylamine. Method [1] Retention time 1.72 min by HPLC and 1.79 min by MS (M+=229).

### EXAMPLE 165

### [3-[1-(3-TERT-BUTYL-PHENYL)-2-METHYL-CYCLOHEXYLAMINO]-1-(3,5-DIFLUORO-BENZYL)-2-HYDROXY-PROPYL]-CARBAMIC ACID TERT-BUTYL ESTER

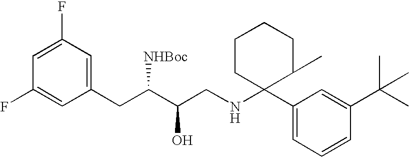

Method [1] Retention time 2.49 min by HPLC and 2.59 min by MS (M+=545).

### EXAMPLE 166

### 3-AMINO-1-[1-(3-TERT-BUTYL-PHENYL)-2-METHYL-CYCLOHEXYLAMINO]-4-(3,5-DIFLUORO-PHENYL)-BUTAN-2-OL

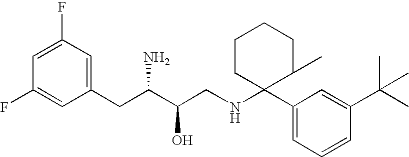

Method [1] Retention time 1.88 min by HPLC and 1.98 min by MS (M+=445).

### EXAMPLE 167

### N[3-[1-(3-TERT-BUTYL-PHENYL)-2-METHYL-CYCLOHEXYLAMINO]-1-(3,5-DIFLUORO-BENZYL)-2-HYDROXY-PROPYL]-ACETAMIDE

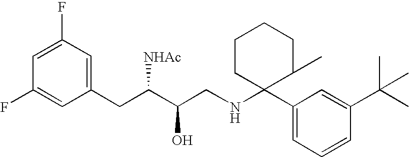

Method [1] Retention time 2.00 min by HPLC and 2.08 min by MS (M+=487).

### EXAMPLE 168

### PREPARATION OF 3-OXO-CYCLOHEXANECARBOXYLIC ACID 2-TRIMETHYLSILANYL-ETHYL ESTER

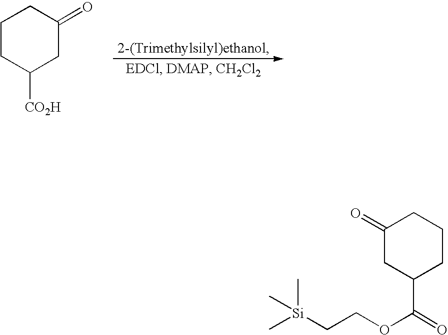

A solution of 3-oxo-cyclohexanecarboxylic acid (2.00 g, 14.1 mmol), 2-trimethylsilylethanol (2.5 mL, 17.4 mmol), 4-dimethylaminopyridine (148 mg, 1.21 mmol), and 1-(3-dimethylaminopropyl)-3-ethylcarbodiimide hydrochloride (3.44 g, 17.9 mmol) in methylene chloride (14 mL) was stirred for 18 h. The solution was diluted with 10% aqueous hydrochloric acid and extracted with methylene chloride. The 20 combined organic extracts were dried over magnesium sulfate, filtered, and concentrated to yield 3.41 g (100% yield) of 3-oxo-cyclohexanecarboxylic acid 2-trimethylsilanyl-ethyl ester as a clear oil. ¹H NMR (300 MHz, CDCl₃); δ 4.16 (m, 2H), 2.75 (m, 1H), 2.55 (d, J=7.9 Hz, 2H), 2.36 (m, 2H), 2.08 (m, 2H), 1.82 (m, 2H, 0.98 (m, 2H), 0.04 (s, 9H).

### EXAMPLE 169

### PREPARATION OF 3-METHYLENE-CYCLOHEXANECARBOXYLIC ACID 2-TRIMETHYLSILANYL-ETHYL ESTER

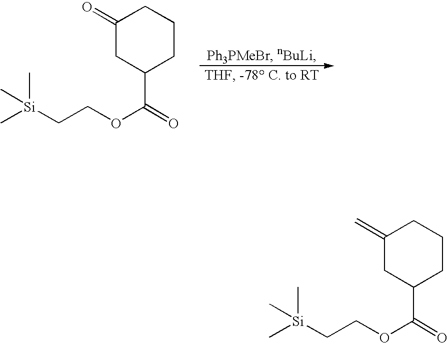

A solution of 1.6 M ⁿbutyllithium in hexanes (14.0 mL, 22.4 mmol) was added to a heterogeneous mixture of methyltriphenylphosphonium bromide (8.02 g, 22.4 mmol) in tetrahydrofuran (50 mL) at −10° C. After stirring for 30 min at −10° C., the yellow slurry was cooled to −78° C. and 3-oxo-cyclohexanecarboxylic acid 2-trimethylsilanyl-ethyl ester (3.41 mg, 14.1 mmol) in tetrahydrofuran (20 mL) was added. After stirring for 10 min at −78° C., the dry ice/acetone bath was removed and the heterogeneous mixture was stirred for 3 h, during which time the solution warmed to ambient temperature. The heterogeneous mixture was concentrated and the residue was flash chromatographed with 99:1, 49:1, 24:1, and 23:2 hexanes/ethyl acetate as the eluant to yield 3.38 g (100% yield) of 3-methylene-cyclohexanecarboxylic acid 2-trimethylsilanyl-ethyl ester as a clear oil. ¹H NMR (300 MHz, CDCl₃); δ 4.68 (s, 2H), 4.16 (m, 2H), 2.51 (m, 1H), 2.24 (broad m, 3H), 1.98 (m, 2H), 1.86 (m, 1H), 1.55 (m, 1H), 1.38 (m, 1H), 0.98 (m, 2H), 0.05 (s, 9H).

### EXAMPLE 170

### PREPARATION OF 1-(3-TERT-BUTYL-PHENYL)-3-METHYLENE-CYCLOHEXANECARBOXYLIC ACID 2-TRIMETHYLSILANYL-ETHYL ESTER

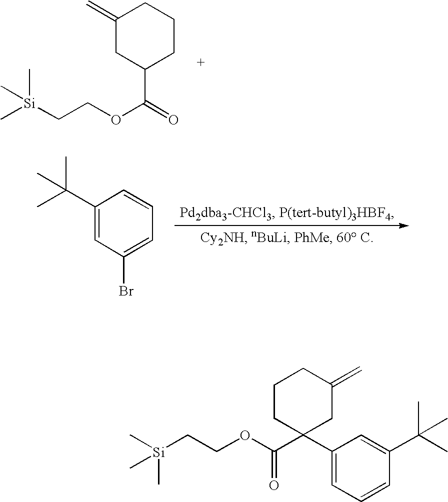

A 1.6 M solution of ⁿbutyllithium (12.0 mL, 19.2 mmol) was added to a solution of dicyclohexylamine (3.7 mL, 18.6 mmol) in toluene (40 mL). After stirring for 5 min, 3-methylene-cyclohexanecarboxylic acid 2-trimethylsilanyl-ethyl ester (3.45 g, 14.4 mmol) was added. After stirring for 30 min, 1-bromo-3-tert-butyl-benzene (3.16 g, 14.8 mmol) was added followed by the simultaneous addition of tri-tert-butylphosphonium tetrafluoroborate (220 mg, 758 μmol) and tris(dibenzylideneacetone)dipalladium(0)-chloroform adduct (380 mg, 367 μmol). The solution was placed into a preheated oil bath at 60° C. After stirring for 16 h, the solution was directly flash chromatographed with 99:1, 49:1, 24:1, and 23:2 hexanes/ethyl acetate as the eluant to yield 4.31 g (81% yield) of 1-(3-tert-butyl-phenyl)-3-methylene-cyclohexanecarboxylic acid 2-trimethylsilanyl-ethyl ester as a light yellow oil. ¹H NMR (300 MHz, CDCl₃); δ 7.43 (d, J=1.0 Hz, 1H), 7.25 (m, 3H), 4.82 (s, 1H), 4.78 (s, 1H), 4.12 (m, 2H), 3.06 (d, J=13.3 Hz, 1H), 2.52 (d, J=13.3 Hz, 2H), 2.26 (dt, J=13.1 Hz and 4.5 Hz, 1H), 2.05 (m, 1H), 1.88-1.59 (broad m, 3H), 1.31 (s, 9H), 0.89 (m, 2H), −0.04 (s, 9H).

### EXAMPLE 171

### PREPARATION OF 1-(3-TERT-BUTYL-PHENYL)-3-METHYLENE-CYCLOHEXANECARBOXYLIC ACID

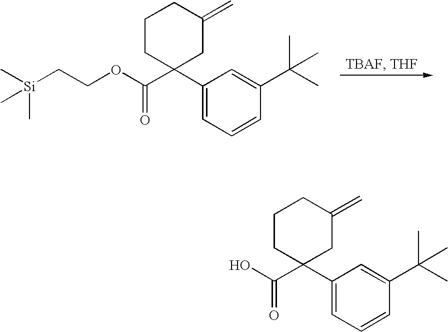

A 1.0 M solution of tetrabutylammonium fluoride in tetrahydrofuran (15.0 mL, 15.0 mmol) was added to 1-(3-tert-butyl-phenyl)-3-methylene-cyclohexanecarboxylic acid 2-trimethylsilanyl-ethyl ester (2.67 mg, 7.16 mmol). After stirring for 16 h, the solution was concentrated, diluted with 10% aqueous hydrochloric acid, and extracted with diethyl ether. The combined organic extracts were dried over magnesium sulfate, filtered, and concentrated to yield 2.09 g (100% yield) of 1-(3-tert-butyl-phenyl)-3-methylene-cyclohexanecarboxylic acid as yellow oil. ¹H NMR (300 MHz, CDCl₃); δ 7.50 (m, 1H), 7.29 (m, 3H), 4.84 (s, 1H), 4.79 (s, 1H), 3.06 (d, J=13.3 Hz, 1H), 2.58 (d, J=13.3 Hz, 1H), 2.51-1.20 (broad m, 6H), 1.34 (s, 9H).

### EXAMPLE 172

### PREPARATION OF 1-(3-TERT-BUTYL-PHENYL)-3-METHYLENE-CYCLOHEXYLAMINE

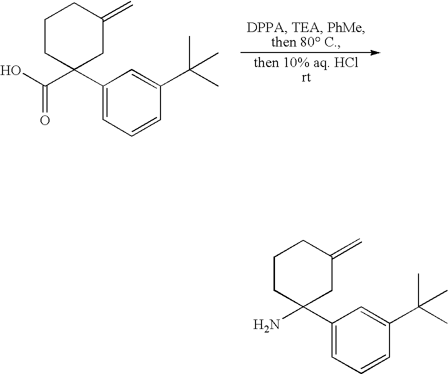

Diphenylphosphoryl azide (0.53 mL, 2.46 mmol) was added to a solution of 1-(3-tert-butyl-phenyl)-3-methylene-cyclohexanecarboxylic acid (554 mg, 2.03 mmol) and triethylamine (0.43 mL, 3.08 mmol) in toluene (4 mL). After stirring at ambient temperature for 18 h, the solution was placed into a preheated oil bath at 80° C. Bubbling was observed. After stirring for 1 h at 80° C., the bubbling had ceased and the solution was cooled to ambient temperature. 10% aqueous hydrochloric acid was added and stirred vigorously for 3 h. The aqueous layer was made alkaline with aqueous 3 N NaOH and extracted with methylene chloride. The combined organic extracts were dried over magnesium sulfate, filtered, and concentrated. The residue was flash chromatographed with 99:1:0.1, 49:1:0.1, 24:1:0.1, and 23:2:0.2 methylene chloride/methanol/concentrated ammonium hydroxide as the eluant to yield 12 mg (2% yield) of 1-(3-tert-butyl-phenyl)-3-methylene-cyclohexylamine. Method [1] Retention time 1.94 min by HPLC and 2.00 min by MS (M−NH₂=227).

### EXAMPLE 173

### [3-[1-(3-TERT-BUTYL-PHENYL)-3-METHYLENE-CYCLOHEXYLAMINO]-1-(3,5-DIFLUORO-BENZYL)-2-HYDROXY-PROPYL]-CARBAMIC ACID TERT-BUTYL ESTER

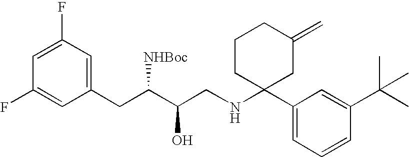

Method [1] Retention time 2.27 min by HPLC and 2.33 min by MS (M+=543).

### EXAMPLE 174

### 3-AMINO-1-[1-(3-TERT-BUTYL-PHENYL)-3-METHYLENE-CYCLOHEXYLAMINO]-4-(3,5-DIFLUORO-PHENYL)-BUTAN-2-OL

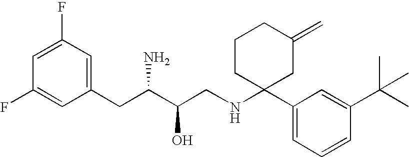

Method [1] Retention time 1.65 min by HPLC and 1.70 min by MS (M+=443).

### EXAMPLE 175

### A[3-[1-(3-TERT-BUTYL-PHENYL)-3-METHYLENE-CYCLOHEXYLAMINO]-1-(3,5-DIFLUORO-BENZYL)-2-HYDROXY-PROPYL]-ACETAMIDE

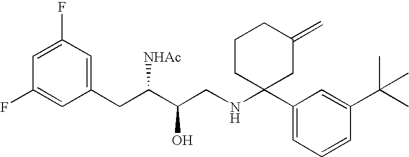

Method [1] Retention time 1.92 min by HPLC and 1.98 min by MS (M+=485).

### EXAMPLE 176

### PREPARATION OF CIS/TRANS N-[3-[1-(3-TERT-BUTYL-PHENYL)-3-HYDROXY-3-HYDROXYMETHYL-CYCLOHEXYLAMINO]-1-(3,5-DIFLUORO-BENZYL)-2-HYDROXY-PROPYL]-ACETAMIDE

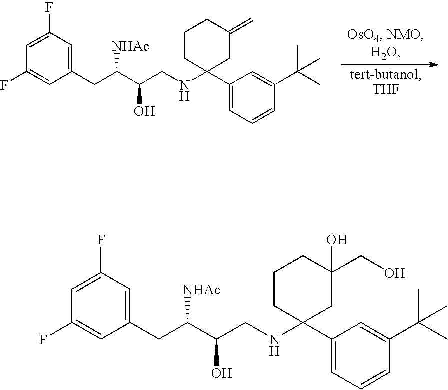

A 4% aqueous solution of osmium tetraoxide (0.75 mL, 123 μmol) was added to a solution of N-[3-[1-(3-tert-Butyl-phenyl)-3-methylene-cyclohexylamino]-1-(3,5-difluoro-benzyl)-2-hydroxy-propyl]-acetamide (200 mg, 413 μmol), and 4-methylmorpholine N-oxide (268 mg, 2.29 mmol) in 2-methyl-2-propanol (3 mL), tetrahydrofuran (0.9 mL), and water (0.3 mL). After stirring for 7 h, sodium sulfite was added, stirred for 30 min, and concentrated. The residue was flash chromotographed with 19:1:0.1, 9:1:0.1, 17:3:0.3, 4:1:0.1, 3:1:1, and 7:3:0.3 methylene chloride/methanol/concentrated ammonium hydroxide as the eluant to yield a mixture of cis/trans isomers of N-[3-[1-(3-tert-Butyl-phenyl)-3-hydroxy-3-hydroxymethyl-cyclohexylamino]-1-(3,5-difluoro-benzyl)-2-hydroxy-propyl]-acetamide. Method [1] Retention time 1.48 min by HPLC and 1.54 min by MS (M+=519). Method [1] Retention time 1.59 min by HPLC and 1.66 min by MS (M+=519).

### EXAMPLE 177

### PREPARATION OF 1-(2,5-DIBROMO-THIOPHEN-3-YL)-CYCLOHEXANECARBOXYLIC ACID METHYL ESTER

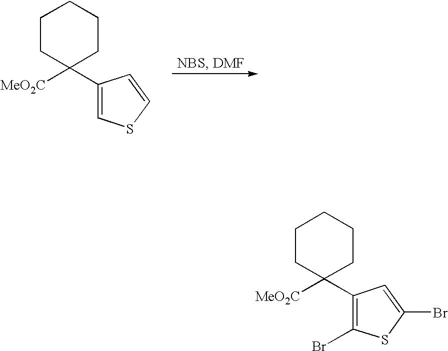

A solution of N-bromosuccinimide (5.58 g, 31.4 mmol) and 1-thiophen-3-yl-cyclohexanecarboxylic acid methyl ester (3.19 g, 14.2 mmol) in dimethylformamide (60 mL) was stirred for 72 h. The solution was diluted with 10% aqueous hydrochloric acid and extracted with diethyl ether. The combined organic extracts were dried over magnesium sulfate, filtered, and concentrated. The residue was flash chromatographed with 99:1, 49:1, and 24:1 hexanes/ethyl acetate as the eluant to yield 4.30 g (79% yield) of 1-(2,5-dibromo-thiophen-3-yl)-cyclohexanecarboxylic acid methyl ester as a yellow oil. ¹H NMR (300 MHz, CDCl₃); δ 6.93 (s, 1 H), 3.67 (s, 3H), 2.34 (m, 2H), 1.90 (m, 2H), 1.60 (m, 5H), 1.36 (m, 1H).

### EXAMPLE 178

### PREPARATION OF 1-(2-BROMO-5-TRIMETHYLSILANYLETHYNYL-THIOPHEN-3-YL)-CYCLOHEXANECARBOXYLIC ACID METHYL ESTER

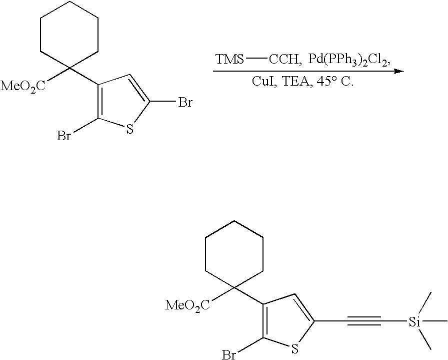

Trimethylsilylacetylene (487 mg, 4.96 mmol), cuprous iodide (55 mg, 289 umol), dichlororbis(triphenylphosphine)palladium(II) (310 mg, 442 μmol), and 1-(2,5-dibromo-thiophen-3-yl)-cyclohexanecarboxylic acid methyl ester (1.71 g, 4.48 mmol) in triethylamine (20 mL) were placed into a preheat oil bath at 45° C. After stirring for 18 h, the solution was diluted with 10% aqueous hydrochloric acid and extracted with diethyl ether. The combined organic extracts were dried over magnesium sulfate, filtered, and concentrated. The residue was flash chromatographed with 99:1, 49:1, and 24:1 hexanes/ethyl acetate as the eluant to yield 1.66 g (93% yield) of 1-(2-bromo-5-trimethylsilanylethynyl-thiophen-3-yl)-cyclohexanecarboxylic acid methyl ester as a yellow solid. ¹H NMR (300 MHz, CDCl₃); δ 7.09 (s, 1 H), 3.67 (s, 3H), 2.34 (m, 2H), 1.93 (m, 2H), 1.58 (m, 5H), 1.35 (m, 1H), 0.23 (s, 9H).

### EXAMPLE 179

### PREPARATION OF 1-(2-BROMO-5-ETHYNYL-THIOPHEN-3-YL)-CYCLOHEXANECARBOXYLIC ACID METHYL ESTER

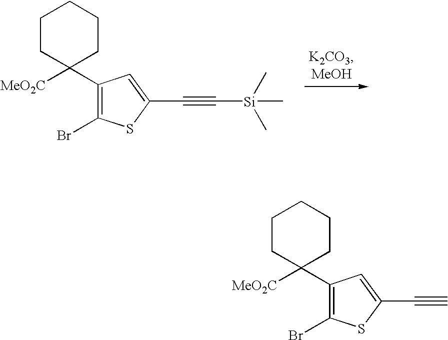

A heterogeneous mixture of potassium carbonate (1.42 g, 10.3 mmol) and 1-(2-bromo-5-trimethylsilanylethynyl-thiophen-3-yl)-cyclohexanecarboxylic acid methyl ester (1.66 g, 4.16 mmol) in methanol (10 mL) was stirred for 24 h. The solution was diluted with water and extracted with methylene chloride. The combined organic extracts were dried over magnesium sulfate, filtered, and concentrated. The residue was flash chromatographed with 99:1, 49:1, and 24:1 hexanes/ethyl acetate as the eluant to yield 1.17 g (74% yield) of 1-(2-bromo-5-ethynyl-thiophen-3-yl)-cyclohexanecarboxylic acid methyl ester as a yellow oil. ¹H NMR (300 MHz, CDCl₃); δ 7.12 (s, 1H), 3.68 (s, 3H), 3.36 (s, 1H), 2.34 (m, 2H), 1.92 (m, 2H), 1.53 (m, 5H), 1.37 (m, 1H).

### EXAMPLE 180

### PREPARATION OF 1-(5-ETHYL-THIOPHEN-3-YL)-CYCLOHEXANECARBOXYLIC ACID METHYL ESTER

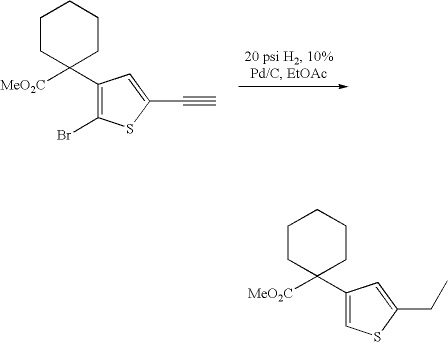

A solution 1-(2-bromo-5-ethynyl-thiophen-3-yl)-cyclohexanecarboxylic acid methyl ester (1.17 g, 3.58 mmol) of in ethyl acetate (20 mL) was added to a heterogeneous mixture of 10% palladium on carbon (1.16 g) and triethylamine (1.5 mL, 10.8 mmol) in ethyl acetate (20 mL) in a parr bottle. The parr bottle was filled with hydrogen (20 psi) and evacuated three times. The parr bottle was refilled with hydrogen (20 psi) and shook for 1.5 h, filtered through celite, and concentrated. The residue was flash chromatographed with 49:1 and 24:1 hexanes/ethyl acetate to yield 813 mg (90% yield) of 1-(5-ethyl-thiophen-3-yl)-cyclohexanecarboxylic acid methyl ester as a clear oil. ¹H NMR (300 MHz, CDCl₃); δ 6.86 (d, J=1.5 Hz, 1H), 6.76 (d, J=1.0 Hz, 1H), 3.66 (s, 3H), 2.79 (dq, J=1.0 Hz and 7.5 Hz, 2H), 2.44 (m, 2H), 1.78-1.19 (broad m, 8H), 1.28 (t, J=7.5 Hz, 3H).

### EXAMPLE 181

### PREPARATION OF 1-(5-ETHYL-THIOPHEN-3-YL)-CYCLOHEXANECARBOXYLIC ACID

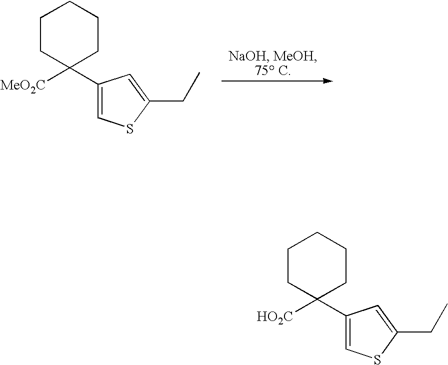

A 3 N solution of aqueous sodium hydroxide (6.0 mL, 18.0 mmol) was added to a solution of 1-(5-ethyl-thiophen-3-yl)-cyclohexanecarboxylic acid methyl ester (813 mg, 3.22 mmol) in methanol (12 mL) and was placed into a preheated oil bath at 75° C. After heating at reflux for 24 h, the solution was concentrated, diluted with 10% aqueous hydrochloric acid, and extracted with methylene chloride. The combined organic extracts were dried over magnesium sulfate, filtered, and concentrated to yield 771 mg (100% yield) of 1-(5-ethyl-thiophen-3-yl)-cyclohexanecarboxylic acid as a white solid. ¹H NMR (300 MHz, CDCl₃); δ 6.92 (d, J=1.5 Hz, 1H), 6.82 (d, J=1.2 Hz, 1H), 2.81 (dq, J=1.2 Hz and 7.5 Hz, 2H), 2.42 (m, 2H), 1.61 (m, 8H), 1.29 (t, J=7.5 Hz, 3H).

### EXAMPLE 182

### PREPARATION OF 1-(5-ETHYL-THIOPHEN-3-YL)-CYCLOHEXYLAMINE

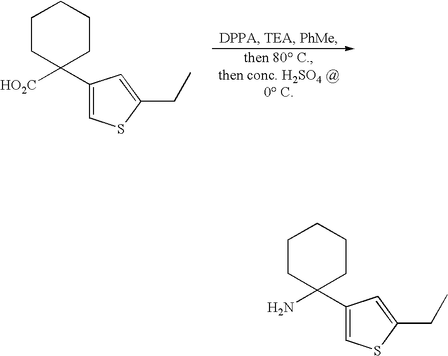

Diphenylphosphoryl azide (0.83 mL, 3.85 mmol) was added to a solution of a 1-(5-ethyl-thiophen-3-yl)-cyclohexanecarboxylic acid and triethylamine (0.67 mL, 4.81 mmol) in toluene (6 mL). After stirring at ambient temperature for 18 h, the solution was placed into a preheated oil bath at 80° C. Bubbling was observed. After stirring for 3 h at 80° C, the bubbling had ceased and the solution was cooled to ambient temperature. Concentrated sulfuric acid was added and stirred vigorously for 2 min. The aqueous layer was made alkaline with aqueous 3 N NaOH and extracted with methylene chloride. The combined organic extracts were dried over magnesium sulfate, filtered, and concentrated. The residue was flash chromatographed with 49:1:0.1, 24:1:0.1, 23:2:0.2, and 22:3:0.3 methylene chloride/methanol/concentrated ammonium hydroxide as the eluant to yield 105 mg of a 1-(5-ethyl-thiophen-3-yl)-cyclohexylamine. Method [1] Retention time 1.23 min by HPLC and 1.29 min by MS (M−NH₂=193).

### EXAMPLE 183

### {1-(3,5-DIFLUORO-BENZYL)-3-[1-(5-ETHYL-THIOPHEN-3-YL)-CYCLOHEXYLAMINO]-2-HYDROXY-PROPYL}-CARBAMIC ACID TERT-BUTYL ESTER

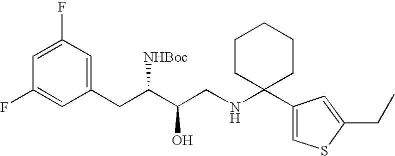

Method [1] Retention time 2.01 min by HPLC and 2.06 min by MS (M+=509).

### EXAMPLE 184

### 3-AMINO-4-(3,5-DIFLUORO-PHENYL)-1-[1-(5-ETHYL-THIOPHEN-3-YL)-CYCLOHEXYLAMINO]-BUTAN-2-OL

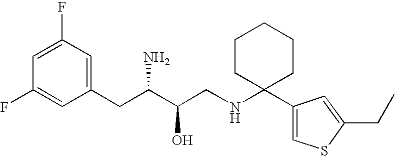

Method [1] Retention time 1.42 min by HPLC and 1.48 min by MS (M+=409).

### EXAMPLE 185

### N{1-(3,5-DIFLUORO-BENZYL)-3-[1-(5-ETHYL-THIOPHEN-3-YL)-CYCLOHEXYLAMINO]-2-HYDROXY-PROPYL}-ACETAMIDE

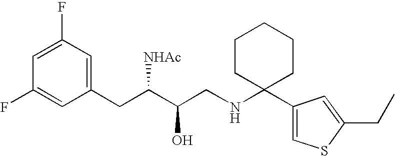

Method [1] Retention time 1.67 min by HPLC and 1.72 min by MS (M+=451).

### EXAMPLE 186

### PREPARATION OF 1-(2,5-DIBROMO-THIOPHEN-3-YL)-CYCLOHEXANECARBOXYLIC ACID

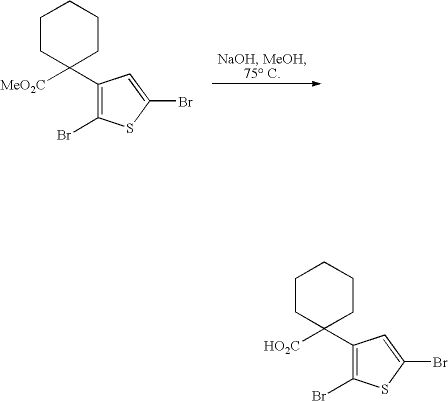

A 3 N solution of aqueous sodium hydroxide (10.0 mL, 30.0 mmol) was added to a solution of 1-(2,5-dibromo-thiophen-3-yl)-cyclohexanecarboxylic acid methyl ester (1.23 g, 3.22 mmol) in methanol (30 mL) and was placed into a preheated oil bath at 75° C. After heating at reflux for 24 h, the solution was concentrated, diluted with 10% aqueous hydrochloric acid, and extracted with methylene chloride. The combined organic extracts were dried over magnesium sulfate, filtered, and concentrated to yield 1.18 mg (100% yield) of 1-(2,5-dibromo-thiophen-3-yl)-cyclohexanecarboxylic acid as yellow oil.

### EXAMPLE 187

### PREPARATION OF 1-(2,5-DIBROMO-THIOPHEN-3-YL)-CYCLOHEXYLAMINE

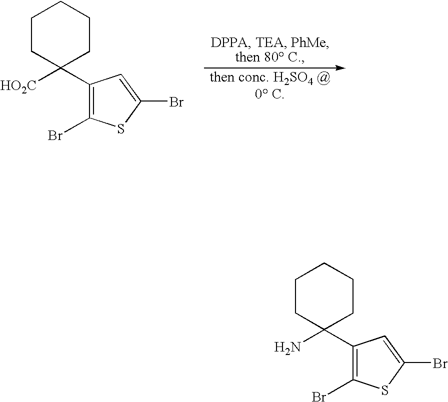

Diphenylphosphoryl azide (0.84 mL, 3.89 mmol) was added to a solution of a 1-(2,5-dibromo-thiophen-3-yl)-cyclohexanecarboxylic acid (1.18 g, 3.21 mmol) and triethylamine (0.68 mL, 4.88 mmol) in toluene (6 mL). After stirring at ambient temperature for 18 h, the solution was placed into a preheated oil bath at 80° C. Bubbling was observed. After stirring for 3 h at 80° C., the bubbling had ceased and the solution was cooled to ambient temperature. Concentrated sulfuric acid was added and stirred vigorously for 2 min. The aqueous layer was made alkaline with aqueous 3 N NaOH and extracted with methylene chloride. The combined organic extracts were dried over magnesium sulfate, filtered, and concentrated. The residue was flash chromatographed with 49:1:0.1, 24:1:0.1, 23:2:0.2, and 22:3:0.3 methylene chloride/methanol/concentrated ammonium hydroxide as the eluant to yield 610 mg (56% yield) of a 1-(2,5-dibromo-thiophen-3-yl)-cyclohexylamine as a brown oil. Method [1] Retention time 1.31 min by HPLC and 1.37 min by MS (M+=321, 323, and 325).

### EXAMPLE 188

### [3-[1-(2,5-DIBROMO-THIOPHEN-3-YL)-CYCLOHEXYLAMINO]-1-(3,5-DIFLUORO-BENZYL)-2-HYDROXY-PROPYL]-CARBAMIC ACID TERT-BUTYL ESTER

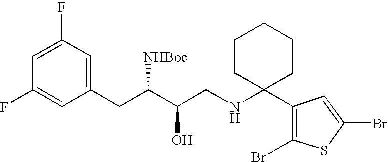

Method [1] Retention time 2.16 min by HPLC and 2.23 min by MS (M+=637, 639, and 641).

### EXAMPLE 189

### 3-AMINO-1-[1-(2,5-DIBROMO-THIOPHEN-3-YL)-CYCLOHEXYLAMINO]-4-(3,5-DIFLUORO-PHENYL)-BUTAN-2-OL

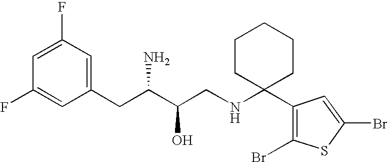

Method [1] Retention time 1.53 min by HPLC and 1.59 min by MS (M+=537, 539, and 541).

### EXAMPLE 190

### M[3-[1-(2,5-DIBROMO-THIOPHEN-3-YL)-CYCLOHEXYLAMINO]-1-(3,5-DIFLUORO-BENZYL)-2-HYDROXY-PROPYL]-ACETAMIDE

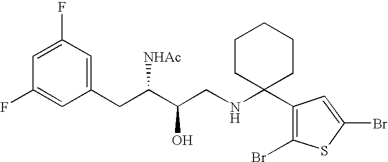

Method [1] Retention time 1.75 min by HPLC and 1.81 min by MS (M+=579, 581, and 583).

### EXAMPLE 191

### PREPARATION OF 1-(5-ACETYL-2-BROMO-THIOPHEN-3-YL)-CYCLOHEXANECARBOXYLIC ACID METHYL ESTER

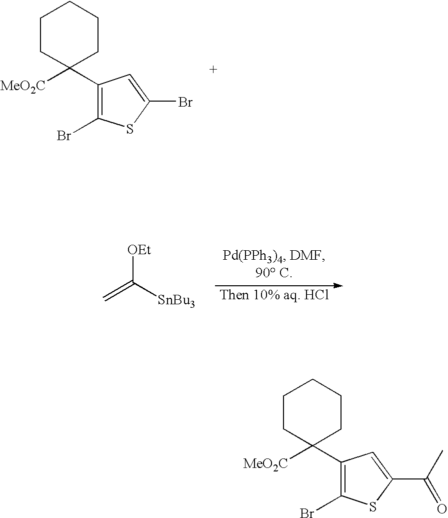

Tetrakis(triphenylphosphine)palladium(0) (380 mg, 329 mmol) was added to a solution of 1-(2,5-dibromo-thiophen-3-yl)-cyclohexanecarboxylic acid methyl ester (1.21 g, 3.17 mmol) and tributyl-(1-ethoxy-vinyl)-stannane (1.33 mg, 3.68 mmol) in dimethylformamide (15 mL) and placed into a preheated oil bath at 90° C. After stirring for 18 h, the solution was cooled to ambient temperature and 10% aqueous hydrochloric acid was added. After stirring for 4 h, the solution was extracted with diethyl ether, the combined organic extracts were dried over magnesium sulfate, filtered, and concentrated. The residue was flash chromatographed with 99:1, 49:1, 24:1, 23:2, 22:3, 21:4, and 4:1 hexanes/ethyl acetate as the eluant to yield 391 mg (impure) of 1-(5-acetyl-2-bromo-thiophen-3-yl)-cyclohexanecarboxylic acid methyl ester. Method [2] Retention time 2.53 min by HPLC and 2.59 min by MS (M+=345 and 347).

### EXAMPLE 192

### PREPARATION OF 1-(2-BROMO-5-ISOPROPENYL-THIOPHEN-3-YL)-CYCLOHEXANECARBOXYLIC ACID METHYL ESTER

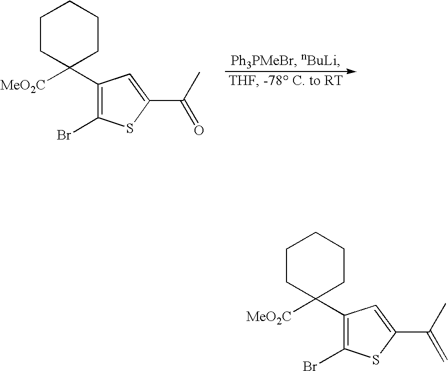

A solution of 1.6 M ⁿbutyllithium in hexanes (2.0 mL, 3.2 mmol) was added to a heterogeneous mixture of methyltriphenylphosphonium bromide (1.14 g, 3.19 mmol) in tetrahydrofuran (10 mL) at −10° C. After stirring for 30 min at −10° C., the yellow slurry was cooled to −78° C. and 1-(5-acetyl-2-bromo-thiophen-3-yl)-cyclohexanecarboxylic acid methyl ester (391 mg, <1.13 mmol, impure) was added. After stirring for 10 min at −78° C., the dry ice/acetone bath was removed and the heterogeneous mixture was stirred for 3 h, during which time the solution warmed to ambient temperature. The heterogeneous mixture was concentrated and the residue was flash chromatographed with 99:1, 49:1, 24:1, and 23:2 hexanes/ethyl acetate as the eluant to yield 268 mg (impure) of 1-(2-bromo-5-isopropenyl-thiophen-3-yl)-cyclohexanecarboxylic acid methyl ester.

### EXAMPLE 193

### PREPARATION OF 1-(5-ISOPROPYL-THIOPHEN-3-YL)-CYCLOHEXANECARBOXYLIC ACID METHYL ESTER

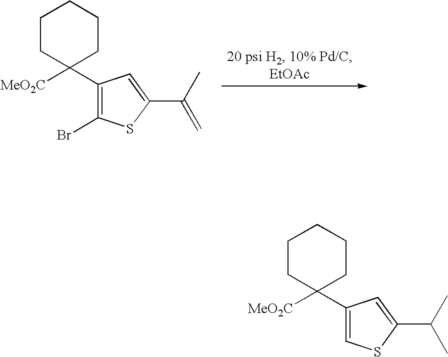

A solution 1-(2-bromo-5-isopropenyl-thiophen-3-yl)-cyclohexanecarboxylic acid methyl ester (268 mg g, <781 μmol, impure) of in ethyl acetate (5 mL) was added to a heterogeneous mixture of 10% palladium on carbon (100 mg) in ethyl acetate (5 mL) in a parr bottle. The parr bottle was filled with hydrogen (20 psi) and evacuated three times. The parr bottle was refilled with hydrogen (20 psi) and shook for 1.5 h, filtered through celite, and concentrated. The residue was flash chromatographed with 49:1 and 24:1 hexanes:ethyl acetate to yield 220 mg (impure) of 1-(5-isopropyl-thiophen-3-yl)-cyclohexanecarboxylic acid methyl ester as a clear oil. ¹H NMR (300 MHz, CDCl₃); δ 6.86 (d, J=1.5 Hz, 1 H), 6.76 (m, 1H), 3.66 (s, 3H), 3.11 (m, 1H), 2.44 (m, 2H), 1.68 (m, 8H), 1.32 (d, J=6.8 Hz, 6H).

### EXAMPLE 194

### PREPARATION OF 1-(5-ISOPROPYL-THIOPHEN-3-YL)-CYCLOHEXANECARBOXYLIC ACID

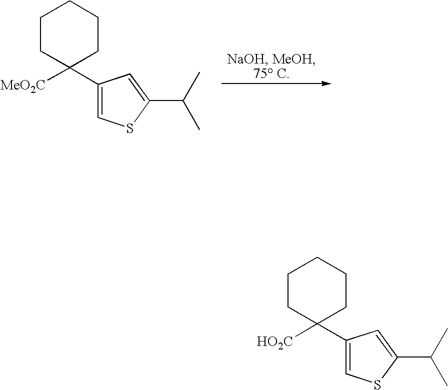

A 3 N solution of aqueous sodium hydroxide (3.0 mL, 9.00 mmol) was added to a solution of 1-(5-isopropyl-thiophen-3-yl)-cyclohexanecarboxylic acid methyl ester (212 mg, <796 μmol, impure) in methanol (10 mL) and was placed into a preheated oil bath at 75° C. After heating at reflux for 24 h, the solution was concentrated, diluted with 10% aqueous hydrochloric acid, and extracted with methylene chloride. The combined organic extracts were dried over magnesium sulfate, filtered, and concentrated to yield 204 mg (impure) of 1-(5-isopropyl-thiophen-3-yl)-cyclohexanecarboxylic acid.

### EXAMPLE 195

### PREPARATION OF 1-(5-ISOPROPYL-THIOPHEN-3-YL)-CYCLOHEXYLAMINE

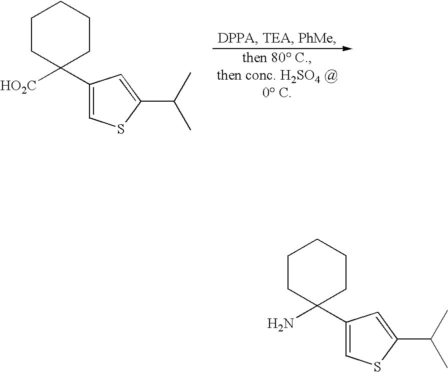

Diphenylphosphoryl azide (0.22 mL, 1.02 mmol) was added to a solution of a 1-(5-isopropyl-thiophen-3-yl)-cyclohexanecarboxylic acid (204 mg, <808 μmol, impure) and triethylamine (0.17 mL, 1.22 mmol) in toluene (2 mL). After stirring at ambient temperature for 18 h, the solution was placed into a preheated oil bath at 80° C. Bubbling was observed. After stirring for 3 h at 80° C., the bubbling had ceased and the solution was cooled to ambient temperature. Concentrated sulfuric acid was added and stirred vigorously for 2 min. The aqueous layer was made alkaline with aqueous 3 N NaOH and extracted with methylene chloride. The combined organic extracts were dried over magnesium sulfate, filtered, and concentrated. The residue was flash chromatographed with 49:1:0.1, 24:1:0.1, 23:2:0.2, and 22:3:0.3 methylene chloride/methanol/concentrated ammonium hydroxide as the eluant to yield 28 mg (16% yield) of a 1-(5-isopropyl-thiophen-3-yl)-cyclohexylamine. Method [1] Retention time 1.41 min by HPLC and 1.47 min by MS (M−NH₂=207).

### EXAMPLE 196

### {1-(3,5-DIFLUORO-BENZYL)-2-HYDROXY-3-[1-(5-ISOPROPYL-THIOPHEN-3-YL)-CYCLOHEXYLAMINO]-PROPYL}-CARBAMIC ACID TERT-BUTYL ESTER

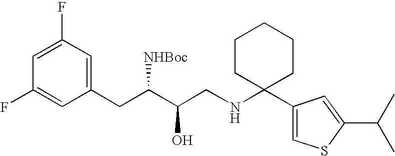

Method [1] Retention time 2.16 min by HPLC and 2.22 min by MS (M+=523).

### EXAMPLE 197

### 3-AMINO-4-(3,5-DIFLUORO-PHENYL)-1-[1-(5-ISOPROPYL-THIOPHEN-3-YL)-CYCLOHEXYLAMINO]-BUTAN-2-OL

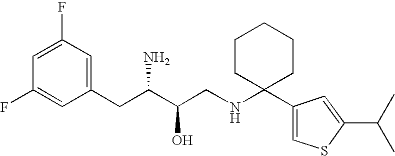

Method [1] Retention time 1.54 min by HPLC and 1.60 min by MS (M+=423).

### EXAMPLE 198

### N-{1-(3,5-DIFLUORO-BENZYL)-2-HYDROXY-3-[1-(5-ISOPROPYL-THIOPHEN-3-YL)-CYCLOHEXYLAMINO]-PROPYL}-ACETAMIDE

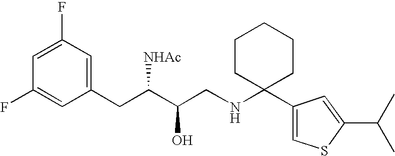

Method [1] Retention time 1.78 min by HPLC and 1.84 min by MS (M+=465).

### EXAMPLE 199

### PREPARATION OF [3-[1-(3-TERT-BUTYL-PHENYL)-CYCLOHEXYLAMINO]-1-(3,5-DIFLUORO-BENZYL)-2-HYDROXY-PROPYL]-UREA

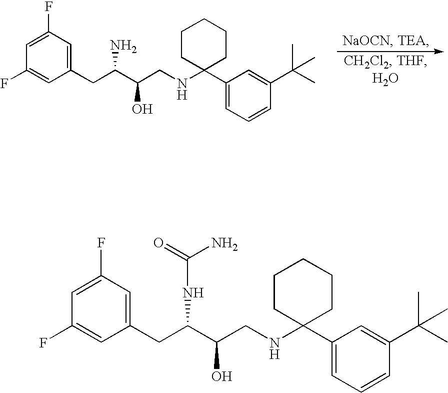

Sodium cyanate (32 mg, 492 umol) was added to a solution of 3-amino-1-[1-(3-tert-butyl-phenyl)-cyclohexylamino]-4-(3,5-difluoro-phenyl)-butan-2-ol dihydrochloride salt (200 mg, 397 umol) and triethylamine (0.08 mL, 574 umol) in methylene chloride (2 mL) and water (2 mL). Three additional portions of sodium cyanate (200 mg, 3.08 mmol) were added after each subsequent 24 h period. After stirring for 4 d, the solution was concentrated and the residue was flash chromotographed with 99:1:0.1, 49:1:0.1, 24:1:0.1, 23:2:0.2, 22:3:0.3, 21:4:0.4, 4:1:0.1, 7:3:0.3, and 3:2:0.2 methylene chloride:methanol:concentrated ammonium hydroxide as the eluant to yield [3-[1-(3-tert-butyl-phenyl)-cyclohexylamino]-1-(3,5-difluoro-benzyl)-2-hydroxy-propyl]-urea. Method [1] Retention time 1.87 min by HPLC and 1.92 min by MS (M+=474).

### EXAMPLE 200

### PREPARATION OF TRIBUTYL-(3-TERT-BUTYL-PHENYL)-STANNANE

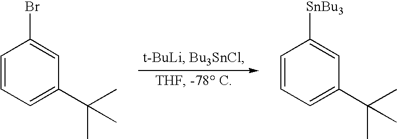

A 1.7 M solution of tert-butyllithium in pentane (2.60 mL, 4.42 mmol) was added to a solution of 1-bromo-3-tert-butyl-benzene (426 mg, 2.00 mmol) in tetrahydrofuran (5 mL) at −78° C. After stirring for 1 h, tributyltin chloride (0.57 mL, 2.10 mmol) was added at −78° C. After stirring for 18 h, during which time the solution warmed to ambient temperature, the solution was diluted with water and extracted with methylene chloride. The combined organic extracts were dried over magnesium sulfate, filtered, and concentrated to yield 976 mg (115% yield) of tributyl-(3-tert-butyl-phenyl)-stannane as a impure light yellow oil.

### EXAMPLE 201

### PREPARATION OF TRIACETOXY-(3-TERT-BUTYL-PHENYL)-LEAD

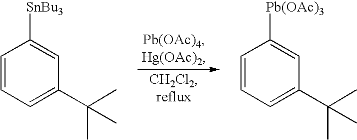

Lead tetraacetate (902 mg, 2.03 mmol) and mercuric acetate (15 mg, 47.1 mmol) was simultaneously added to a solution of tributyl-(3-tert-butyl-phenyl)-stannane (ca. 2.00 mmol) in methylene chloride (4 mL) and was placed into a preheated oil bath at 45° C. After heating at reflux for 24 h, the solution was cooled to ambient temperature and filtered through celite. The celite was washed with chloroform and the filtrate was concentrated to yield the triacetoxy-(3-tert-butyl-phenyl)-lead as an off white/light yellow solid.

### EXAMPLE 202

### PREPARATION OF 2-(3-TERT-BUTYL-PHENYL)-2-NITRO-CYCLOHEXANONE

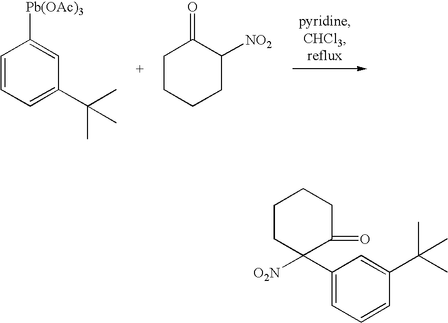

Pyridine (1.8 mL, 22.3 mmol) and 2-nitro-cyclohexanone (630 mg, 4.40 mmol) in chloroform (5 mL) was stirred for 15 min. Triacetoxy-(3-tert-butyl-phenyl)-lead (<2.00 mmol) in chloroform (5 mL) was added and the solution was placed into a preheated oil bath at 85° C. After heating at reflux for 16 h, the solution was concentrated and the residue was flash chromatographed with 19:1, 9:1, and 17:3 hexanes:ethyl acetate as the eluant to yield 160 mg (28% over three steps) of 2-(3-tert-butyl-phenyl)-2-nitro-cyclohexanone as a yellow oil. ¹H NMR (300 MHz, CDCl₃); δ 7.48 (d, J=7.7 Hz, 1H), 7.39 (m, 1H), 7.34 (s, 1H), 7.15 (d, J=7.2 Hz, 1H), 3.06 (m, 1H), 2.94 (m, 1H), 2.54 (m, 2H), 1.95 (m, 3H), 1.74 (m, 1H), 1.32 (s, 9H). Method [2] Retention time 1.74 min by HPLC and 1.79 min by MS (M+Na=298).

### EXAMPLE 203

### PREPARATION OF CIS/TRANS 2-AMINO-2-(3-TERT-BUTYL-PHENYL)-CYCLOHEXANOL

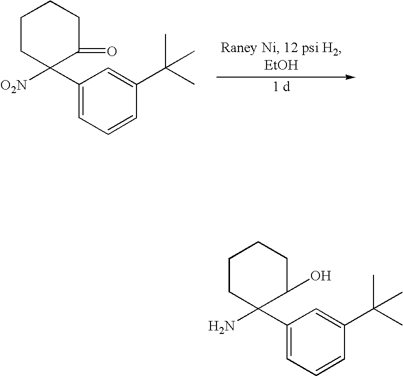

Raney 2800 nickel slurry in water (2 mL) was added to a solution of 2-(3-tert-butyl-phenyl)-2-nitro-cyclohexanone (40 mg, 145 μmol) in ethanol (10 mL) in a parr bottle. The parr bottle was filled with hydrogen (12 psi) and evacuated three times. The parr bottle was refilled with hydrogen (12 psi) and shook for 18 h. The heterogeneous mixture was filtered through celite and concentrated to yield a mixture of cis/trans isomers of 2-amino-2-(3-tert-butyl-phenyl)-cyclohexanol. Method [1] Retention time 1.38 min by HPLC and 1.43 min by MS (M−NH₂=231).

### EXAMPLE 204

### [3-[1-(3-TERT-BUTYL-PHENYL)-2-HYDROXY-CYCLOHEXYLAMINO]-1-(3,5-DIFLUORO-BENZYL)-2-HYDROXY-PROPYL]-CARBAMIC ACID TERT-BUTYL ESTER

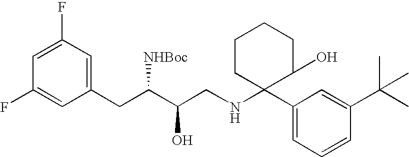

Method [1] Retention time 2.20 min by HPLC and 2.25 min by MS (M+=547).

### EXAMPLE 205

### 2-[3-AMINO-4-(3,5-DIFLUORO-PHENYL)-2-HYDROXY-BUTYLAMINO]-2-(3-TERT-BUTYL-PHENYL)-CYCLOHEXANOL

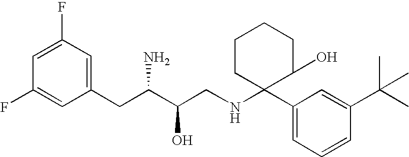

Method [1] Retention time 1.53 min by HPLC and 1.60 min by MS (M+=447).

### EXAMPLE 206

### N-[3-[1-(3-TERT-BUTYL-PHENYL)-2-HYDROXY-CYCLOHEXYLAMINO]-1-(3,5-DIFLUORO-BENZYL)-2-HYDROXY-PROPYL]-ACETAMIDE

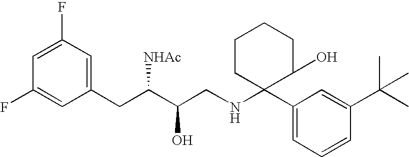

Method [1] Retention time 1.83 min by HPLC and 1.87 min by MS (M+=488).

### EXAMPLE 207

### PREPARATION OF 1-(5-BROMO-THIOPHEN-2-YL)-CYCLOHEXANOL

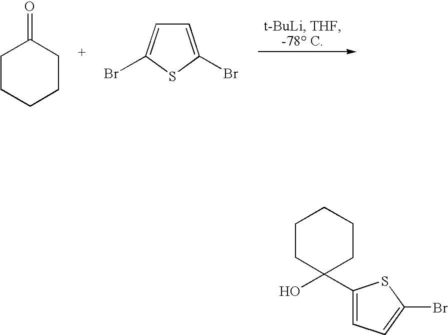

A solution of 1.7 M tert-butyllithium in pentane (14.0 mL, 23.8 mmol) was added to a solution of 2,5-dibromothiophene (2.67 g, 11.0 mmol) in tetrahydrofuran (20 mL) at −78° C. After stirring for 1 h, cyclohexanone (1.4 mL, 13.5 mmol) was added. After stirring for 18 h, during which time the solution warmed to ambient temperature, the solution was diluted with saturated aqueous ammonium chloride and extracted with methylene chloride. The combined organic extracts were dried over magnesium sulfate, filtered, and concentrated. The residue was flash chromatographed with 19:1, 9:1, 17:3, 4:1 and 3:1 hexanes/ethyl acetate as the eluant to yield 2.58 g (90% yield) of 1-(5-bromo-thiophen-2-yl)-cyclohexanol as a light orange oil.

¹H NMR (300 MHz, CDCl₃); δ 6.89 (d, J=3.8 Hz, 1H), 6.72 (d, J=3.8 Hz, 1H), 2.34 (m, 2H), 1.95-1.62 (m, 6H), 1.28 (m, 2H).

### EXAMPLE 208

### PREPARATION OF 2-(1-AZIDO-CYCLOHEXYL)-5-BROMO-THIOPHENE

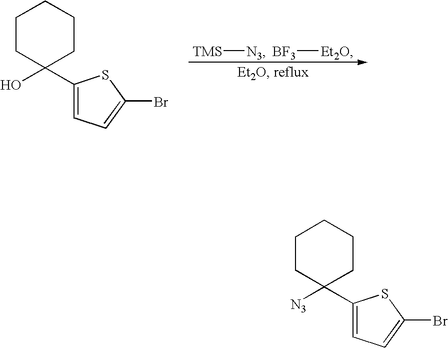

Borontrifluoride-etherate (1.3 mL, 10.3 mmol) was added to a solution of 1-(5-bromo-thiophen-2-yl)-cyclohexanol (2.57 g, 9.84 mmol) and azidotrimethylsilane (2.6 mL, 19.6 mmol) in diethyl ether (20 mL) and placed into a preheated oil bath at 45° C. After heating at reflux for 1.5 h, the solution was diluted with water and extracted with diethyl ether. The combined organic extracts were dried over magnesium sulfate, filtered, and concentrated. The residue was flash chromatographed with 99:1, 49:1, and 24:1 hexanes/ethyl acetate as the eluant to yield 1.29 g (46% yield) of 2-(1-Azido-cyclohexyl)-5-bromo-thiophene as a light yellow oil. ¹H NMR (300 MHz, CDCl₃); δ 6.95 (d, J=3.8 Hz, 1H), 6.79 (d, J=3.8 Hz, 1H), 2.00 (m, 2H), 1.87 (m, 2H), 1.62 (m, 5H), 1.34 (m, 1H).

### EXAMPLE 209

### PREPARATION OF 1-(5-BROMO-THIOPHEN-2-YL)-CYCLOHEXYLAMINE

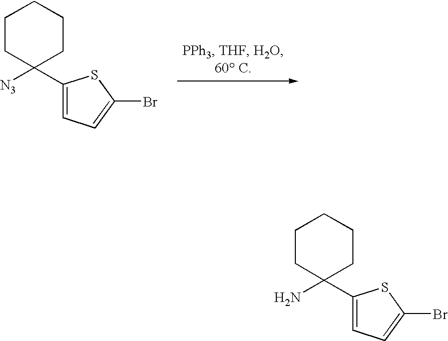

A solution of triphenylphosphine (550 mg, 2.10 mmol) and 2-(1-Azido-cyclohexyl)-5-bromo-thiophene (289 mg, 1.01 mmol) in tetrahydrofuran (5 mL) and water (1 mL) was placed into a preheated oil bath at 60° C. After stirring for 24 h, the solution was concentrated and the residue was flash chromatographed w/49:1:0.1, 24:1:0.1, 23:2:0.2, and 22:3:0.3 methylene chloride/methanol/concentrated ammonium hydroxide as the eluant to yield 1-(5-bromo-thiophen-2-yl)-cyclohexylamine impure with triphenylphosphine oxide. Method [1] Retention time 1.20 min by HPLC and 1.26 min by MS (M−NH₂=243 and 245).

### EXAMPLE 210

### [3-[1-(5-BROMO-THIOPHEN-2-YL)-CYCLOHEXYLAMINO]-1-(3,5-DIFLUORO-BENZYL)-2-HYDROXY-PROPYL]-CARBAMIC ACID TERT-BUTYL ESTER

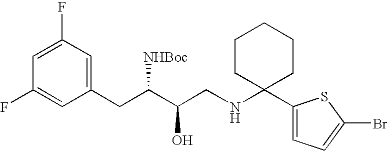

Method [1] Retention time 2.06 min by HPLC and 2.12 min by MS (M+=559 and 561).

### EXAMPLE 211

### 3-AMINO-1-[1-(5-BROMO-THIOPHEN-2-YL)-CYCLOHEXYLAMINO]-4-(3,5-DIFLUORO-PHENYL)-BUTAN-2-OL

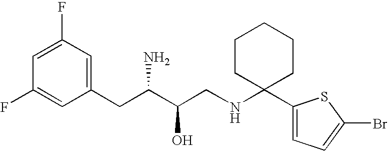

Method [1] Retention time 1.42 min by HPLC and 1.48 min by MS (M+=459 and 461).

### EXAMPLE 212

### N[3-[1-(5-BROMO-THIOPHEN-2-YL)-CYCLOHEXYLAMINO]-1-(3,5-DIFLUORO-BENZYL)-2-HYDROXY-PROPYL]-ACETAMIDE

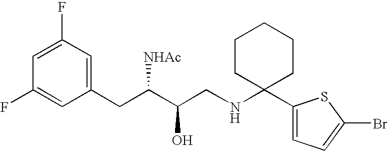

Method [1] Retention time 1.65 min by HPLC and 1.71 min by MS (M+=501 and 503).

### EXAMPLE 213

### PREPARATION OF N-{1-(3,5-DIFLUORO-BENZYL)-2-HYDROXY-3-[1-(5-ISOPROPENYL-THIOPHEN-2-YL)-CYCLOHEXYLAMINO]-PROPYL}-ACETAMIDE

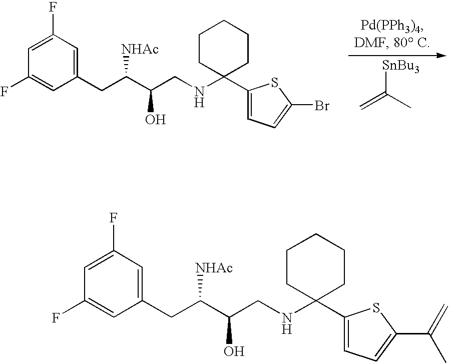

Tetrakis(triphenylphosphine)palladium(0) (77 mg, 66.6 mmol) was added to a solution of N-[3-[1-(5-bromo-thiophen-2-yl)-cyclohexylamino]-1-(3,5-difluoro-benzyl)-2-hydroxy-propyl]-acetamide (260 mg, 519 μmol) and tributyl-isopropenyl-stannane (548 mg, 1.65 mmol) in dimethylformamide (4 mL) and placed into a preheated oil bath at 80° C. After stirring for 18 h, the solution was concentrated and the residue was flash chromatographed with 99:1:0.1, 49:1:0.1, 24:1:0.1, and 23:2:0.2 methylene chloride/methanol/concentrated ammonium hydroxide as the eluant to yield N-{1-(3,5-difluoro-benzyl)-2-hydroxy-3-[1-(5-isopropenyl-thiophen-2-yl)-cyclohexylamino]-propyl}-acetamide. Method [1] Retention time 1.77 min by HPLC and 1.82 min by MS (M+Na=485).

### EXAMPLE 214

### PREPARATION OF N-{1-(3,5-DIFLUORO-BENZYL)-2-HYDROXY-3-[1-(5-ISOPROPYL-THIOPHEN-2-YL)-CYCLOHEXYLAMINO]-PROPYL}-ACETAMIDE

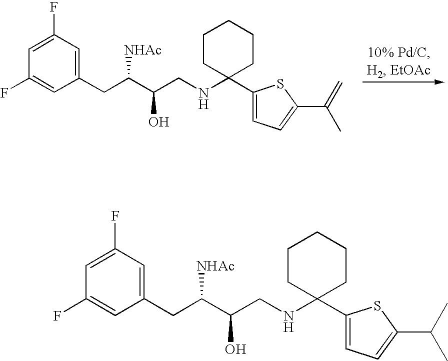

A solution of a N-{1-(3,5-difluoro-benzyl)-2-hydroxy-3-[1-(5-isopropenyl-thiophen-2-yl)-cyclohexylamino]-propyl}-acetamide (120 mg, 259 μmol) in ethyl acetate (10 mL) was added to 10% palladium on carbon (50 mg) in a parr bottle. The parr bottle was filled with hydrogen (15 psi) and evacuated three times. The parr bottle was refilled with hydrogen (15 psi), shook for 1 h, filtered through celite, and concentrated. The residue was flash chromotographed with 99:1:0.1, 49:1:0.1, 24:1:0.1, 23:2:0.2, and 22:3:0.3 methylene chloride/methanol/concentrated ammonium hydroxide as the eluant to yield N-{1-(3,5-difluoro-benzyl)-2-hydroxy-3-[1-(5-isopropyl-thiophen-2-yl)-cyclohexylamino]-propyl}-acetamide. Method [1] Retention time 1.78 min by HPLC and 1.85 min by MS (M+Na=487).

### EXAMPLE 215

### PREPARATION OF N-[1-(3,5-DIFLUORO-BENZYL)-2-HYDROXY-3-(1-THIOPHEN-2-YL-CYCLOHEXYLAMINO)-PROPYL]-ACETAMIDE

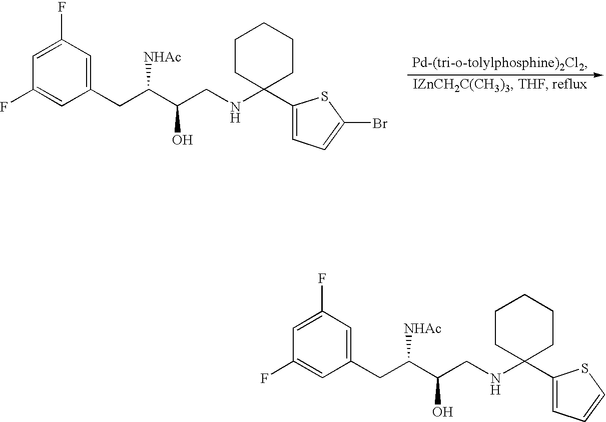

A 0.5 M solution of neopentylzinc iodide in tetrahydrofuran (6.0 mL, 3.00 mmol) was added to N-[3-[1-(5-bromo-thiophen-2-yl)-cyclohexylamino]-1-(3,5-difluoro-benzyl)-2-hydroxy-propyl]-acetamide (175 mg, 349 μmol) and dichlorobis(tri-tolylphosphine)palladium(II) (57 mg, 72.5 μmol) and placed into a preheated oil bath at 70° C. After heating at reflux for 18 h, the solution was concentrated and the residue 99:1:0.1, 49:1:0.1, 24:1:0.1, 23:2:0.2, 22:3:0.3, and 21:4:0.4 methylene chloride/methanol/concentrated ammonium hydroxide as the eluant to yield N-[1-(3,5-difluoro-benzyl)-2-hydroxy-3-(1-thiophen-2-yl-cyclohexylamino)-propyl]-acetamide. Method [1] Retention time 1.48 min by HPLC and 1.54 min by MS (M+=423).

### EXAMPLE 216

### PREPARATION OF N-{1-(3,5-DIFLUORO-BENZYL)-2-HYDROXY-3-[1-(5-TRIMETHYLSILANYLETHYNYL-THIOPHEN-2-YL)-CYCLOHEXYLAMINO]-PROPYL}-ACETAMIDE

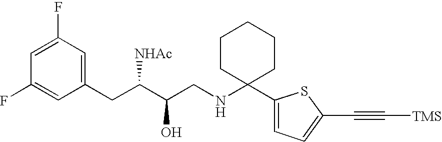

A solution of trimethylsilylacetylene (0.5 mL, 3.54 mmol), cuprous iodide (31 mg, 163 mmol), dichlororbis(triphenylphosphine)palladium(II) (68 mg, 96.9 umol), and N-[3-[1-(5-bromo-thiophen-2-yl)-cyclohexylamino]-1-(3,5-difluoro-benzyl)-2-hydroxy-propyl]-acetamide (300 mg, 598 umol) in triethylamine (5 mL) was placed into a preheated oil bath at 45° C. After stirring for 18 h, the solution was concentrated and the residue was flash chromatographed with 99:1:0.1, 49:1:0.1, and 24:1:0.1 methylene chloride/methanol/concentrated ammonium hydroxide as the eluant to yield N-{1-(3,5-difluoro-benzyl)-2-hydroxy-3-[1-(5-trimethylsilanylethynyl-thiophen-2-yl)-cyclohexylamino]-propyl}-acetamide. Method [1] Retention time 2.18 min by HPLC and 2.25 min by MS (M+=519).

### EXAMPLE 217

### PREPARATION OF N-[3-{1-[5-(1-CHLORO-VINYL)-THIOPHEN-2-YL]-CYCLOHEXYLAMINO}-1-(3,5-DIFLUORO-BENZYL)-2-HYDROXY-PROPYL]-ACETAMIDE

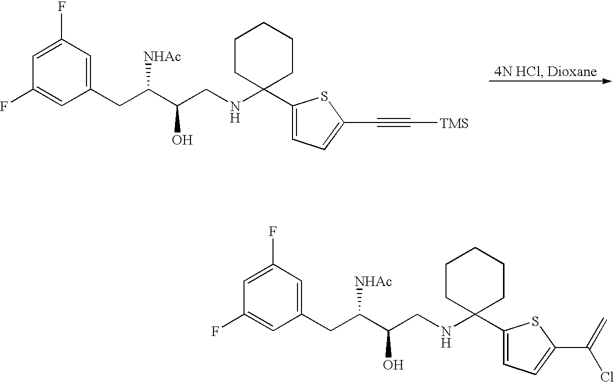

A solution of N-{1-(3,5-difluoro-benzyl)-2-hydroxy-3-[1-(5-trimethylsilanylethynyl-thiophen-2-yl)-cyclohexylamino]-propyl}-acetamide (300 mg, 578 μmol) was stirring in a 4 N solution of hydrochloric acid in dioxane (10 mL) for 6 h. The solution was concentrated and the residue was flash chromatographed with 49:1:0.1, 24:1:0.1, and 23:2:0.2 methylene chloride:methanol:concentrated ammonium hydroxide as the eluant to yield N-[3-{1-[5-(1-chloro-vinyl)-thiophen-2-yl]-cyclohexylamino)-1-(3,5-difluoro-benzyl)-2-hydroxy-propyl]-acetamide. Method [1] Retention time 1.80 min by HPLC and 1.86 min by MS (M+=483 and 485).

### EXAMPLE 218

### PREPARATION OF N-{1-(3,5-DIFLUORO-BENZYL)-3-[1-(5-ETHYL-THIOPHEN-2-YL)-CYCLOHEXYLAMINO]-2-HYDROXY-PROPYL}-ACETAMIDE

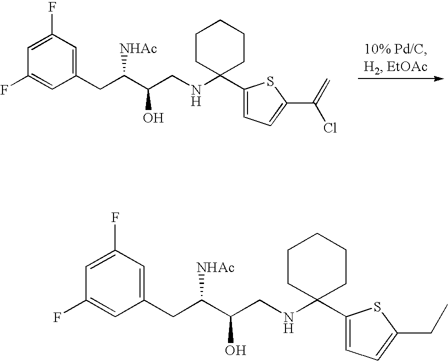

A solution of a mixture of N-[3-{1-[5-(1-chloro-vinyl)-thiophen-2-yl]-cyclohexylamino}-1-(3,5-difluoro-benzyl)-2-hydroxy-propyl]-acetamide in ethyl acetate (10 mL) was added to 10% palladium on carbon (200 mg) in a parr bottle. The parr bottle was filled with hydrogen (12 psi) and evacuated three times. The parr bottle was refilled with hydrogen (12 psi) and shaken for 1 h. The heterogeneous mixture was filtered through celite and concentrated. The residue was flash chromatographed with 49:1:0.1, 24:1:0.1, and 23:2:0.2 methylene chloride/methanol/concentrated ammonium hydroxide as the eluant to yield N-{1-(3,5-difluoro-benzyl)-3-[1-(5-ethyl-thiophen-2-yl)-cyclohexylamino]-2-hydroxy-propyl}-acetamide. Method [1] Retention time 1.69 min by HPLC and 1.75 min by MS (M+=451).

### EXAMPLE 219

### PREPARATION OF 8-METHYLENE-1,4-DIOXA-SPIRO[4.5]DECANE

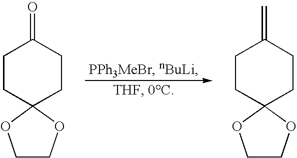

A solution of 1.6 M ⁿbutyllithium in hexanes (46 mL, 73.6 mmol) was slowly added to a heterogeneous mixture of methyltriphenylphosphonium bromide (28.07 g, 78.6 mmol) in tetrahydrofuran (150 mL) at −10° C. After stirring for 1 h, 1,4-dioxa-spiro[4.5]decan-8-one (8.01 g, 51.3 mmol) was added. After stirring for 3 h, during which time the solution warmed to ambient temperature, acetone was added and the heterogeneous mixture was concentrated. The residue was diluted with 1:1 methylene chloride:ethyl ether, filtered and concentrated. The residue was flash chromatographed with 49:1, 24:1, and 23:2 hexanes/ethyl acetate as the eluant to yield 6.22 g (79% yield) of 8-methylene-1,4-dioxa-spiro[4.5]decane as a yellow oil. ¹H NMR (300 MHz, CDCl₃); δ 4.67 (s, 2H), 3.96 (s, 4H), 2.29 (m, 4H), 1.70 (m, 4H).

### EXAMPLE 220

### PREPARATION OF 4-METHYLENE-CYCLOHEXANONE

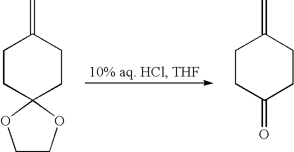

A solution of 8-methylene-1,4-dioxa-spiro[4.5]decane (6.22 g, 40.3 mmol) was stirred in tetrahydrofuran (100 mL) and 10% aqueous hydrochloric acid (100 mL) for 18 h. The solution was extracted with ethyl ether and the combined organic extracts were dried over magnesium sulfate. The combined organic extracts were filtered and concentrated to yiled 3.89 g (88% yield) of 4-methylene-cyclohexanone as a yellow oil. ¹H NMR (300 MHz, CDCl₃); δ 4.89 (s, 2H), 2.47 (m, 8H).

### EXAMPLE 221

### PREPARATION OF 1-(3-TERT-BUTYL-PHENYL)-4-METHYLENE-CYCLOHEXANOL

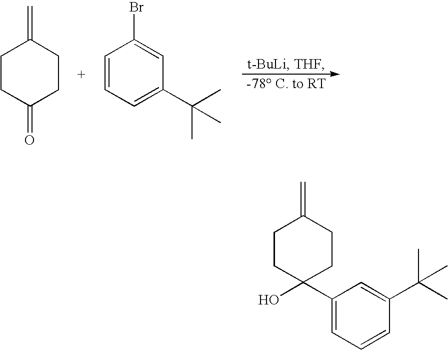

A solution of 1.7 M tert-butyllithium in pentane (32.0 mL, 54.4 mmol) was added to a solution of 1-bromo-3-tert-butyl-benzene (5.54 g, 26.0 mmol) in tetrahydrofuran (60mL) at −78° C. After stirring for 1 h, cyclohexanone (2.00 g, 18.2 mmol) in tetrahydrofuran (15 mL) was added. After stirring for 18 h, during which time the solution warmed to ambient temperature, the solution was diluted with saturated aqueous ammonium chloride and extracted with methylene chloride. The combined organic extracts were dried over magnesium sulfate, filtered, and concentrated. The residue was flash chromatographed with 49:1, 24:1, 23:2 hexanes/ethyl acetate as the eluant to yield 3.61 g (81% yield) of 1-(3-tert-butyl-phenyl)-4-methylene-cyclohexanol as a yellow oil. ¹H NMR (300 MHz, CDCl₃); δ 7.56 (s, 1H), 7.30 (m, 3H), 4.72 (s, 2H), 2.60 (m, 2H), 2.27 (m, 2H), 1.93 (m, 4H), 1.33 (s, 9H).

### EXAMPLE 222

### PREPARATION OF 1-(1-AZIDO-4-METHYLENE-CYCLOHEXYL)-3-TERT-BUTYL-BENZENE

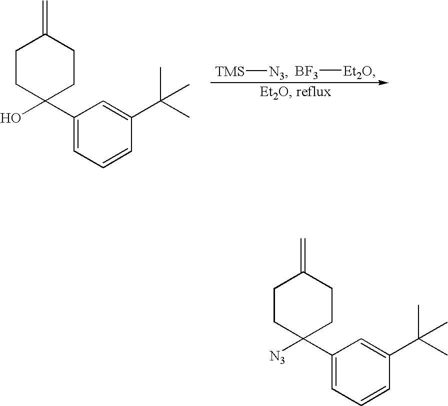

Borontrifluoride-etherate (2.0 mL, 15.7 mmol) was added to a solution of 1-(3-tert-butyl-phenyl)-4-methylene-cyclohexanol (3.60 g, 14.7 mmol) and azidotrimethylsilane (4.0 mL, 30.1 mmol) in diethyl ether (30 mL) and placed into a preheated oil bath at 45° C. After heating at reflux for 4 h, the solution was diluted with saturated aqueous ammonium chloride and extracted with diethyl ether. The combined organic extracts were dried over magnesium sulfate, filtered, and concentrated. The residue was flash chromatographed with 99:1, 49:1, and 24:1 hexanes/ethyl acetate as the eluant to yield 1.46 g (37% yield) of 1-(1-azido-4-methylene-cyclohexyl)-3-tert-butyl-benzene as clear oil. ¹H NMR (300 MHz, CDCl₃); δ 7.47 (s, 1H), 7.36-7.23 (broad m, 3H), 4.72 (s, 2H), 2.48 (m, 2H), 2.28 (m, 2H), 2.13 (m, 2H), 1.96 (m, 2H), 1.34 (s, 9H).

### EXAMPLE 223

### PREPARATION OF 1-(3-TERT-BUTYL-PHENYL)-4-M ETHYLENE-CYCLOHEXYLAMINE

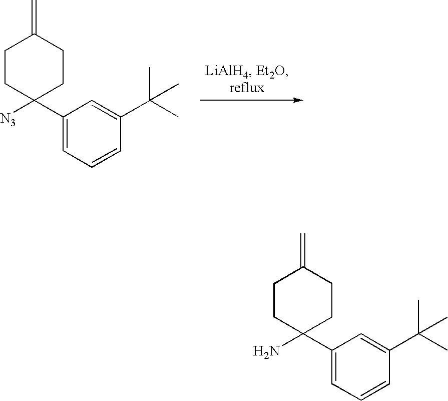

A solution of 1-(1-azido-4-methylene-cyclohexyl)-3-tert-butyl-benzene (820 mg, 3.04 mmol) in diethyl ether (10 mL) was added to a heterogeneous mixture of lithium aluminum hydride (510 mg, 13.4 mmol) in diethyl ether (10 mL) and was placed into a preheated oil bath at 40° C. After heating at reflux for 24 h, the solution was cooled to ambient temperature, and celite and sodium sulfate decahydrate was added. After stirring for 1 h, the heterogenous mixture was filtered through celite to yield 1-(3-tert-butyl-phenyl)-4-methylene-cyclohexylamine. Method [1] Retention time 1.62 min by HPLC and 1.67 min by MS (M+=244).

### EXAMPLE 224

### [3-[1-(3-TERT-BUTYL-PHENYL)-4-METHYLENE-CYCLOHEXYLAMINO]-1-(3,5-DIFLUORO-BENZYL)-2-HYDROXY-PROPYL]-CARBAMIC ACID TERT-BUTYL ESTER

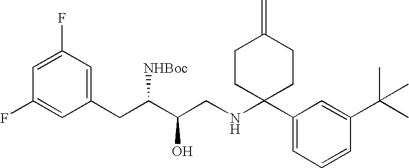

Method [1] Retention time 2.40 min by HPLC and 2.47 min by MS (M+=543).

### EXAMPLE 225

### 3-AMINO-1-[1-(3-TERT-BUTYL-PHENYL)-4-METHYLENE-CYCLOHEXYLAMINO]-4-(3,5-DIFLUORO-PHENYL)-BUTAN-2-OL

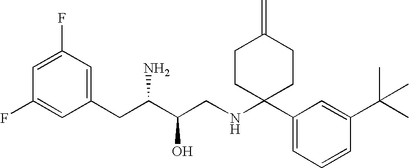

Method [1] Retention time 1.36 min by HPLC and 1.42 min by MS (M+=443).

### EXAMPLE 226

### N-[3-[1-(3-TERT-BUTYL-PHENYL)-4-METHYLENE-CYCLOHEXYLAMINO]-1-(3,5-DIFLUORO-BENZYL)-2-HYDROXY-PROPYL]-ACETAMIDE

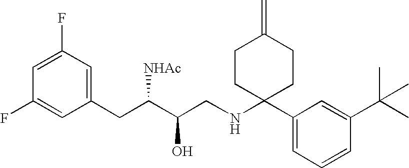

¹H NMR (300 MHz, CDCl₃); δ 9.10 (broad d, 1H), 8.10 (broad d, 1H), 7.61 (s, 1H), 7.40 (broad m, 3H), 6.64 (broad s, 3H), 6.50 (m, 1H), 6.00 (broad s, 3H), 4.72 (s, 1H), 3.98 (broad s, 1H), 3.77 (broad s, 1H), 2.93 (m, 1H), 2.68 (m, 4H), 2.37 (m, 3H), 2.09 (m, 3H), 1.83 (s, 3H), 1.32 (s, 9H). Method [1] Retention time 2.04 min by HPLC and 2.11 min by MS (M+=485).

### EXAMPLE 227

### PREPARATION OF N-[3-[1-(3-TERT-BUTYL-PHENYL)-4-METHYL-CYCLOHEXYLAMINO]-1-(3,5-DIFLUORO-BENZYL)-2-HYDROXY-PROPYL]-ACETAMIDE

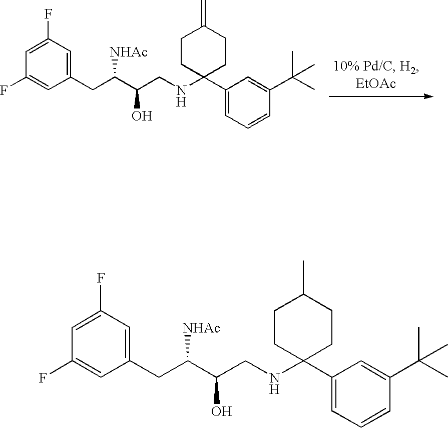

A solution of a mixture of N-[3-[1-(3-tert-butyl-phenyl)-4-methylene-cyclohexylamino]-1-(3,5-difluoro-benzyl)-2-hydroxy-propyl]-acetamide (100 mg, 206 μmol) in ethyl acetate (10 mL) was added to a heterogeneous mixture of 10% palladium on carbon (50 mg) in ethyl acetate (10 mL) in a parr bottle. The parr bottle was filled with hydrogen (20 psi) and evacuated three times. The parr bottle was refilled with hydrogen (20 psi) and shook for 1 h, filtered through celite, and concentrated to yield a single isomer of N-[3-[1-(3-tert-butyl-phenyl)-4-methyl-cyclohexylamino]-1-(3,5-difluoro-benzyl)-2-hydroxy-propyl]-acetamide. ¹H N MR (300 MHz, CDCl₃); δ 9.23 (broad s, 1H), 8.09 (broad s, 1H), 7.59 (s, 1H), 7.37 (broad m, 3H), 6.67 (m, 3H), 6.45 (d, J=8.6 Hz, 1H), 5.84 (broad s, 1H), 3.97 (m, 1H), 3.71 (m, 1H), 2.92 (dd, J=3.8 Hz and 14.2 Hz, 1H), 2.68 (m, 4H), 2.43 (m, 1H), 1.99 (m, 2H), 1.81 (s, 3H), 1.72 (m, 2H), 1.52 (m, 1H), 1.31 (s, 9H), 0.93 (m, 2H), 0.84 (d, J=6.4 Hz, 3H), Method [1] Retention time 2.09 min by HPLC and 2.15 min by MS (M+=487).

### EXAMPLE 228

### PREPARATION OF CIS/TRANS N-[3-[1-(3-TERT-BUTYL-PHENYL)-4-HYDROXY-4-HYDROXYMETHYL-CYCLOHEXYLAMINO]-1-(3,5-DIFLUORO-BENZYL)-2-HYDROXY-PROPYL]-ACETAMIDE

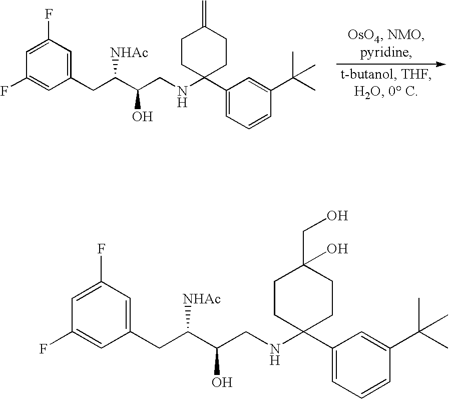

A 4% aqueous solution of osmium tetraoxide (0.67 mL, 110 μmol) was added to a solution of N-[3-[1-(3-tert-butyl-phenyl)-4-methylene-cyclohexylamino]-1-(3,5-difluoro-benzyl)-2-hydroxy-propyl]-acetamide (526 mg, 1.09 mmol), 4-methylmorpholine N-oxide (200 mg, 1.17 mmol), and pyridine (0.02 mL, 247 μmol) in 2-methyl-2-propanol (5 mL) tetrahydrofuran (1.5 mL), and water (0.5 mL) at 0° C. After stirring for 24 h, during which time the biphasic solution warmed to ambient temperature, the solution was diluted with saturated aqueous sodium sulfite and concentrated. The residue was flash chromotographed with 99:1:0.1, 49:1:0.1, 24:1:0.1, 23:2:0.2, 4:1:0.1, 3:1:1, and 7:3:0.3 methylene chloride/methanol/concentrated ammonium hydroxide as the eluant to yield a mixture of cis/trans isomers of N-[3-[1-(3-tert-butyl-phenyl)-4-hydroxy-4-hydroxymethyl-cyclohexylamino]-1-(3,5-difluoro-benzyl)-2-hydroxy-propyl]-acetamide. Method [1] Retention time 1.54 min by HPLC and 1.63 min by MS (M+=519).

### EXAMPLE 229

### PREPARATION OF CIS/TRANS N-[3-[8-(3-TERT-BUTYL-PHENYL)-2-OXO-1,3-DIOXA-SPIRO[4.5]DEC-8-YLAMINO]-1-(3,5-DIFLUORO-BENZYL)-2-HYDROXY-PROPYL]-ACETAMIDE

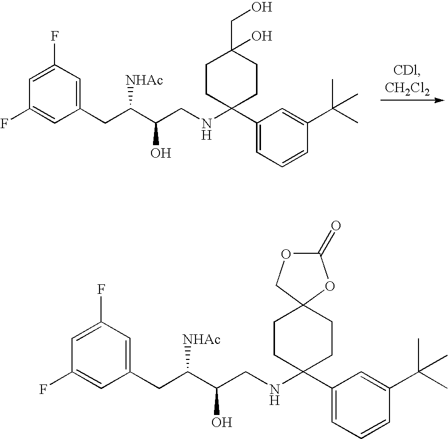

To a solution of a mixture of cis/trans isomers of N-[3-[1-(3-tert-butyl-phenyl)-4-hydroxy-4-hydroxymethyl-cyclohexylamino]-1-(3,5-difluoro-benzyl)-2-hydroxy-propyl]-acetamide (117 mg, 226 μmol) in methylene chloride (2 mL) was added 1,1′-carbonyidiimidazole (44 mg, 271 μmol). Additional portions of 1,1′-carbonyldiimidazole (42 mg, 259 μmol), (37 mg, 228 μmol), and (21 mg, 130 μmol) were added after each subsequent 24 h periods. The solution was directly flash chromatographed with 99:1:0.1, 49:1:0.1, 24:1:0.1, and 23:2:0.2 methylene chloride/methanol/concentrated ammonium hydroxide as the eluant to yield a mixture of cis/trans isomers of N-[3-[8-(3-tert-butyl-phenyl)-2-oxo-1,3-dioxa-spiro[4.5]dec-8-ylamino]-1-(3,5-difluoro-benzyl)-2-hydroxy-propyl]-acetamide. ¹H NMR (300 MHz, CDCl₃); δ 9.75 (broad s, 1H), 8.65 (broad s, 1H), 7.70 (s, 1H), 7.46 (d, J=7.8 Hz, 1H), 7.39 (t, J=7.8 Hz, 1H), 7.30 (d, J=7.8 Hz, 1H), 6.64 (m, 3H), 6.41 (d, J=8.2 Hz, 1H), 4.31 (s, 2H), 4.09 (m, 1H), 3.66 (broad s, 1H), 2.85 (broad m, 4H), 2.42 (broad m, 3H), 2.01 (m, 2H), 2.13 (m, 2H), 1.81 (s, 3H), 1.54 (m, 2H), 1.31 (s, 9H). Method [1] Retention time 1.74 min by HPLC and 1.81 min by MS (M+=545). ¹H NMR (300 MHz, CDCl₃); δ 9.91 (broad s, 1H), 8.45 (broad s, 1H), 7.58 (s, 1H), 7.46 (d, J=7.8 Hz, 1H), 7.39 (t, J=7.8 Hz, 1H), 7.30 (d, J=7.8 Hz, 1H), 6.64 (m, 3H), 5.96 (d, J=8.1 Hz, 1H), 4.31 (s, 2H), 4.09 (broad m, 2H), 3.66 (broad s, 1H), 3.03 (m, 1H), 2.50 (broad m, 7H), 2.01 (m, 2H), 1.85 (m, 2H), 1.81 (s, 3H), 1.31 (s, 9H). Method [1] Retention time 1.82 min by HPLC 1.88 min by MS (M+=545).

### EXAMPLE 230

### PREPARATION OF CIS/TRANS [4-AZIDO-4-(3-TERT-BUTYL-PHENYL)-CYCLOHEXYL]-METHANOL

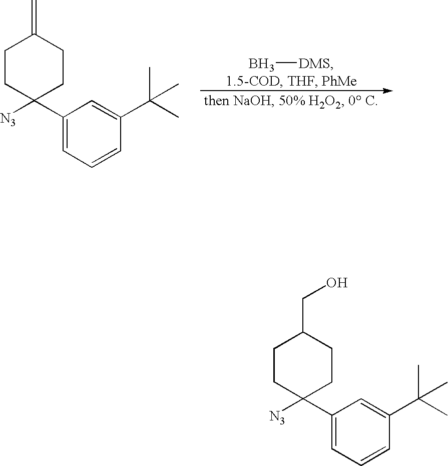

A solution of 2.0 M borane-dimethyl sulfide complex in toluene (1.1 mL, 2.2 mmol) was added to a solution of 1,5-cyclooctadiene (0.28 mL, 2.28 mmol) in tetrahydrofuran (5 mL) and was placed into a preheated oil bath at 70° C. After heating at reflux for 1 h, the solution was cooled to ambient temperature and 1-(1-azido-4-methylene-cyclohexyl)-3-tert-butyl-benzene (559 mg, 2.08 mmol) was added. After stirring for 18 h, the solution was cooled to 0° C. and 3 N aqueous solution of sodium hydroxide (5.0 mL, 15.0 mmol) was added followed by the slow dropwise addition of 50% aqueous hydrogen peroxide (2.0 mL, 34.7 mmol). After stirring for 4 h, during which time the biphasic solution warmed to ambient temperature, the biphasic solution was extracted with methylene chloride. The combined organic extracts were dried over magnesium sulfate, filtered, and concentrated. The residue was flash chromatographed with 9:1, 4:1, and 7:3 hexanes:ethyl acetate as the eluant to yield 469 mg (79% yield) of a mixture of cis/trans isomers of [4-azido-4-(3-tert-butyl-phenyl)-cyclohexyl]-methanol as a clear oil. ¹H NMR (300 MHz, CDCl₃); δ 7.48 (s, 1H), 7.36-7.23 (broad m, 3H), 3.57 and 3.45 (t and m, J=5.5 Hz, 2H), 2.15 (m, 2H), 1.81 (m, 4H), 1.60-1.13 (broad m 3H), 1.34 (s, 9H).

### EXAMPLE 231

### PREPARATION OF CIS/TRANS [4-AMINO-4-(3-TERT-BUTYL-PHENYL)-CYCLOHEXYL]-METHANOL

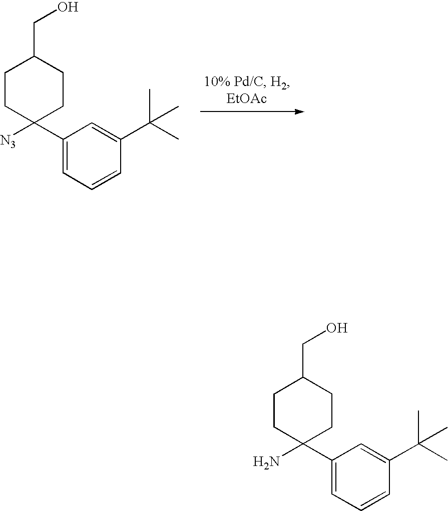

A solution of a mixture of cis/trans isomers of [4-azido-4-(3-tert-butyl-phenyl)-cyclohexyl]-methanol in ethyl acetate (10 mL) was added to a heterogeneous mixture of 10% palladium on carbon (400 mg) in ethyl acetate (10 mL) in a parr bottle. The parr bottle was filled with hydrogen (20 psi) and evacuated three times. The parr bottle was refilled with hydrogen (20 psi) and shook for 1 h, filtered through celite, and concentrated to yield a mixture of cis/trans isomers of [4-amino-4-(3-tert-butyl-phenyl)-cyclohexyl]-methanol. Method [1] Retention time 1.18 min by HPLC and 1.26 min by MS (M−NH₂=245). Method [1] Retention time 1.28 min by HPLC and 1.37 min by MS (M−NH₂=245).

### [3-[1-(3-tert-Butyl-phenyl)-4-HYDROXYMETHYL-CYCLOHEXYLAMINO]-1-(3,5-DIFLUORO-BENZYL)-2-HYDROXY-PROPYL]-CARBAMIC ACID TERT-BUTYL ESTER

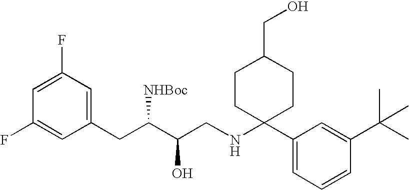

Method [1] Retention time 1.98 min by HPLC and 2.05 min by MS (M+=561). Method [1] Retention time 2.06 min by HPLC and 2.12 min by MS (M+=561).

### EXAMPLE 232

### 3-AMINO-1-[1-(3-TERT-BUTYL-PHENYL)-4-HYDROXYMETHYL-CYCLOHEXYLAMINO]-4-(3,5-DIFLUORO-PHENYL)-BUTAN-2-OL

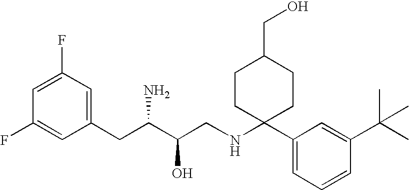

Method [1] Retention time 1.41 min by HPLC and 1.48 min by MS (M+=461).

### EXAMPLE 233

### 3-AMINO-1-[1-(3-TERT-BUTYL-PHENYL)-4-HYDROXYMETHYL-CYCLOHEXYLAMINO]-4-(3,5-DIFLUORO-PHENYL)-BUTAN-2-OL

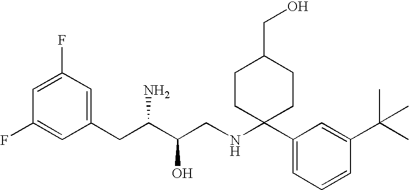

Method [1] Retention time 1.50 min by HPLC and 1.57 min by MS (M+=461).

### EXAMPLE 234: N-[3-[1-(3-TERT-BUTYL-PHENYL)-4-HYDROXYMETHYL-CYCLOHEXYLAMINO]-1-(3,5-DIFLUORO-BENZYL)-2-HYDROXY-PROPYL]-ACETAMIDE

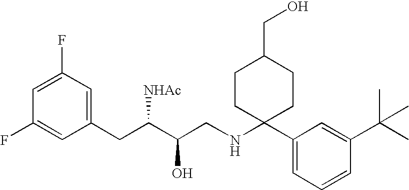

¹H NMR (300 MHz, CDCl₃); δ 9.43 (broad s, 1H), 8.23 (broad s, 1H), 7.60 (s, 1H), 7.37 (broad m, 3H), 6.66 (m, 3H), 6.34 (m, 1H), 3.99 (m, 1H), 3.66 (m, 3H), 3.34 (d, J=6.2 Hz, 2H), 2.68 (broad m, 5H), 2.42 (m, 1H), 2.03 (m, 2H), 1.85 (m, 2H), 1.81 (s, HPLC and 1.67 min by MS (M+=503).

### EXAMPLE 235

### N-[3-[1-(3-TERT-BUTYL-PHENYL)-4-HYDROXYMETHYL-CYCLOHEXYLAMINO]-1-(3,5-DIFLUORO-BENZYL)-2-HYDROXY-PROPYL]-ACETAMIDE

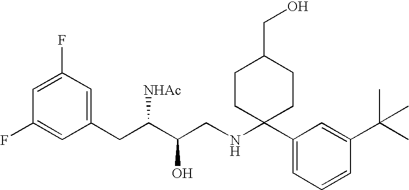

Method [1] Retention time 1.67 min by HPLC and 1.73 min by MS (M+=503).

### EXAMPLE 236

### PREPARATION OF 1-(4-BROMO-THIOPHEN-2-YL)-CYCLOHEXANOL

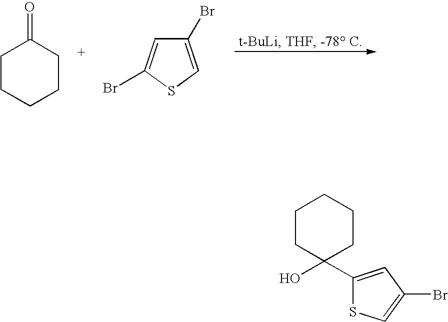

A solution of 1.7 M tert-butyllithium in pentane (39.0 mL, 66.3 mmol) was slowly added along the walls of the flask to a solution of 2,4-dibromothiophene (7.81 g, 32.3 mmol) in tetrahydrofuran (120 mL) at −78° C. After stirring for 1 h, cyclohexanone (4.0 mL, 38.6 mmol) was added. After stirring for 18 h, during which time the solution warmed to ambient temperature, the solution was diluted with saturated aqueous ammonium chloride and extracted with methylene chloride. The combined organic extracts were dried over magnesium sulfate, filtered, and concentrated. The residue was flash chromatographed with 19:1 and 9:1 hexanes:ethyl acetate as the eluant to yield 3.48 g (41% yield) of 1-(4-bromo-thiophen-2-yl)-cyclohexanol as a light yellow oil. ¹H NMR (300 MHz, CDCl₃); δ 7.10 (d, J=0.9 Hz, 1H), 6.88 (d, J=0.9 Hz, 1H), 2.21 (m, 1H), 1.93-1.57 (broad m, 7H), 1.29 (m, 2H).

### EXAMPLE 237

### PREPARATION OF 2-(1-AZIDO-CYCLOHEXYL)-4-BROMO-THIOPHENE

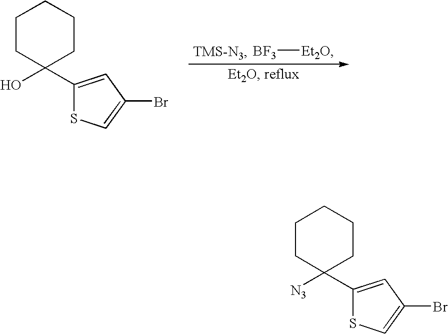

Borontrifluoride-etherate (2.0 mL, 15.8 mmol) was added to a solution of 1-(4-bromo-thiophen-2-yl)-cyclohexanol (3.48 g, 13.3 mmol) and azidotrimethylsilane (3.5 mL, 26.4 mmol) in diethyl ether (25 mL) and placed into a preheated oil bath at 45° C. After heating at reflux for 4 h, the solution was diluted with water and extracted with diethyl ether. The combined organic extracts were dried over magnesium sulfate, filtered, and concentrated. The residue was flash chromatographed with 99:1, 49:1, and 24:1 hexanes:ethyl acetate as the eluant to yield 2.89 g (impure) of 2-(1-azido-cyclohexyl)-4-bromo-thiophene as a oil. ¹H NMR (300 MHz, CDCl₃); δ 7.19 (d, J=1.4 Hz, 1H), 6.94 (d, J=1.4 Hz, 1H), 2.10 (m, 3H), 1.87 (m, 1H), 1.67 (m, 5H), 1.34 (m, 1H).

### EXAMPLE 238

### PREPARATION OF 1-(4-BROMO-THIOPHEN-2-YL)-CYCLOHEXYLAMINE

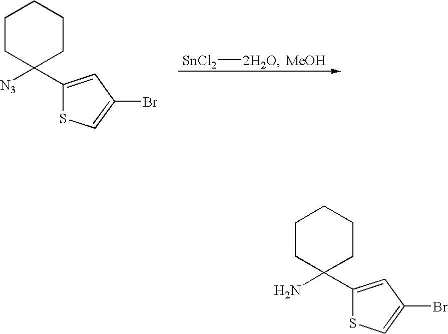

Tin(II) chloride dihydrate (3.43 g, 15.2 mmol) was added to a solution of 2-(1-Azido-cyclohexyl)-4-bromo-thiophene (1.74 g, 6.00 mmol) in methanol (20 mL). After stirring for 4 h, 3 N aqueous sodium hydroxide was added. After stirring for 16 h, the solution was extracted with methylene chloride. The combined organic extracts were filtered through celite, dried over magnesium sulfate, filtered and concentrated to yield 1-(4-bromo-thiophen-2-yl)-cyclohexylamine. Method [1] Retention time 1.15 min by HPLC and 1.21 min by MS (M−NH₂=243 and 245).

### EXAMPLE 239

### [3-[1-(4-BROMO-THIOPHEN-2-YL)-CYCLOHEXYLAMINO]-1-(3,5-DIFLUORO-BENZYL)-2-HYDROXY-PROPYL]-CARBAMIC ACID TERT-BUTYL ESTER

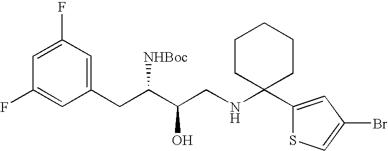

Method [1] Retention time 2.03 min by HPLC and 2.11 min by MS (M+=559 and 561).

### EXAMPLE 240

### 3-AMINO-1-[1-(4-BROMO-THIOPHEN-2-YL)-CYCLOHEXYLAMINO]-4-(3,5-DIFLUORO-PHENYL)-BUTAN-2-OL

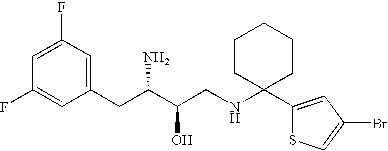

Method [1] Retention time 1.41 min by HPLC and 1.48 min by MS (M+=459 and 461).

### EXAMPLE 241

### N-[3-[1-(4-BROMO-THIOPHEN-2-YL)-CYCLOHEXYLAMINO]-1-(3,5-DIFLUORO-BENZYL)-2-HYDROXY-PROPYL]-ACETAMIDE

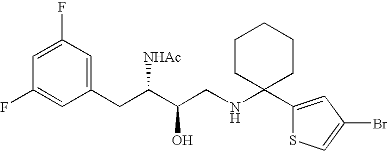

Method [1] Retention time 1.58 min by HPLC and 1.64 min by MS (M+=501 and 503).

### EXAMPLE 242

### PREPARATION OF N-{1-(3,5-DIFLUORO-BENZYL)-2-HYDROXY-3-[1-(4-ISOPROPENYL-THIOPHEN-2-YL)-CYCLOHEXYLAMINO]-PROPYL}-ACETAMIDE

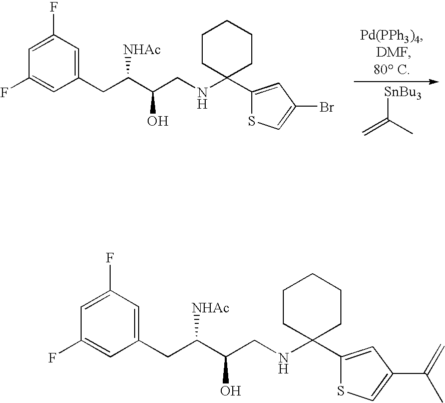

Tetrakis(triphenylphosphine)palladium(0) (107 mg, 92.6 mmol) was added to a solution of N-[3-[1-(4-bromo-thiophen-2-yl)-cyclohexylamino]-1-(3,5-difluoro-benzyl)-2-hydroxy-propyl]-acetamide (213 mg, 425 umol) and tributyl-isopropenyl-stannane (800 mg, 2.42 mmol) in dimethylformamide (4 mL) and placed into a preheated oil bath at 80° C. After stirring for 18 h, the solution was concentrated and the residue was flash chromatographed with 99:1:0.1, 49:1:0.1, 24:1:0.1, and 23:2:0.2 methylene chloride:methanol:concentrated ammonium hydroxide as the eluant to yield N-{1-(3,5-difluoro-benzyl)-2-hydroxy-3-[1-(4-isopropenyl-thiophen-2-yl)-cyclohexylamino]-propyl}-acetamide. Method [1] Retention time 1.73 min by HPLC and 1.80 min by MS (M+=463).

### EXAMPLE 243

### PREPARATION OF N-{1-(3,5-DIFLUORO-BENZYL)-2-HYDROXY-3-[1-(4-ISOPROPYL-THIOPHEN-2-YL)-CYCLOHEXYLAMINO]-PROPYL}-ACETAMIDE

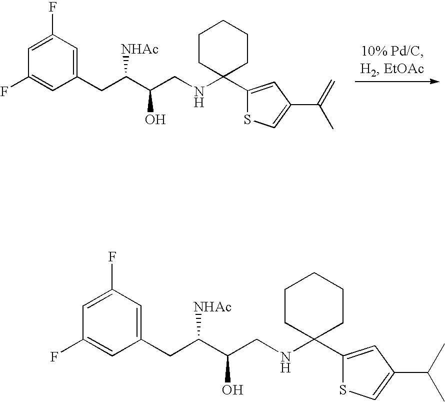

A solution of a mixture of N-{1-(3,5-difluoro-benzyl)-2-hydroxy-3-[1-(4-isopropenyl-thiophen-2-yl)-cyclohexylamino]-propyl)-acetamide in ethyl acetate (10 mL) was added to of 10% palladium on carbon (200 mg) in a parr bottle. The parr bottle was filled with hydrogen (12 psi) and evacuated three times. The parr bottle was refilled with hydrogen (12 psi) and shook for 1 h. The heterogeneous mixture filtered through celite and concentrated. The residue was flash chromatographed with 49:1:0.1, 24:1:0.1, and 23:2:0.2 methylene chloride:methanol:concentrated ammonium hydroxide as the eluant to yield N-{1-(3,5-difluoro-benzyl)-2-hydroxy-3-[1-(4-isopropyl-thiophen-2-yl)-cyclohexylamino]-propyl}-acetamide. Method [1] Retention time 1.85 min by HPLC and 1.93 min by MS (M+=465).

### EXAMPLE 244

### PREPARATION OF {1-(3,5-DIFLUORO-BENZYL)-2-HYDROXY-3-[1-(4-TRIMETHYLSILANYLETHYNYL-THIOPHEN-2-YL)-CYCLOHEXYLAMINO]-PROPYL}-CARBAMIC ACID TERT-BUTYL ESTER

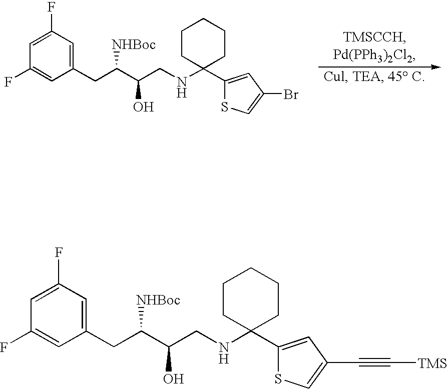

Trimethylsilylacetylene (2.0 mL, 14.2 mmol), cuprous iodide (83 mg, 436 mmol), dichlororbis(triphenylphosphine)palladium(II) (350 mg, 499 umol), and [3-[1-(4-bromo-thiophen-2-yl)-cyclohexylamino]-1-(3,5-difluoro-benzyl)-2-hydroxy-propyl]-carbamic acid tert-butyl ester (0.94 g, 1.68 mmol) in triethylamine (20 mL) was placed into a preheat oil bath at 45° C. After stirring for 18 h, the solution was concentrated and the residue was flash chromatographed with 99:1:0.1, 49:1:0.1, and 24:1:0.1 methylene chloride:methanol:concentrated ammonium hydroxide as the eluant to yield {1-(3,5-difluoro-benzyl)-2-hydroxy-3-[1-(4-trimethylsilanylethynyl-thiophen-2-yl)-cyclohexylamino]-propyl}-carbamic acid tert-butyl ester. Method [1] Retention time 2.40 min by HPLC and 2.46 min by MS (M+=577).

### EXAMPLE 245

### 3-AMINO-4-(3,5-DIFLUORO-PHENYL)-1-[1-(4-TRIMETHYLSILANYLETHYNYL-THIOPHEN-2-YL)-CYCLOHEXYLAMINO]-BUTAN-2-OL

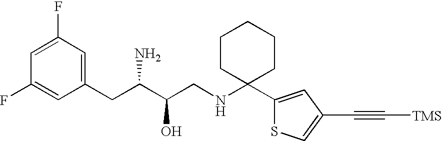

Method [1] Retention time 1.78 min by HPLC and 1.84 min by MS (M+=477).

### EXAMPLE 246

### N{1-(3,5-DIFLUORO-BENZYL)-2-HYDROXY-3-[1-(4-TRIMETHYLSILANYLETHYNYL-THIOPHEN-2-YL)-CYCLOHEXYLAMINO]-PROPYL}-ACETAMIDE

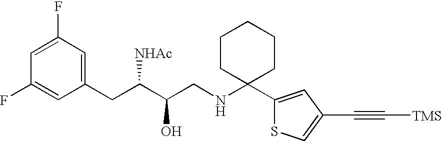

Method [1] Retention time 2.10 min by HPLC and 2.16 min by MS (M+=519).

### EXAMPLE 247

### PREPARATION OF N-{1-(3,5-DIFLUORO-BENZYL)-3-[1-(4-ETHYNYL-THIOPHEN-2-YL)-CYCLOHEXYLAMINO]-2-HYDROXY-PROPYL}-ACETAMIDE

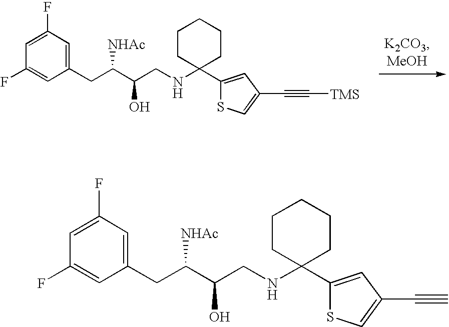

A heterogeneous mixture of potassium carbonate (276 mg, 2.00 mmol) and N-{1-(3,5-difluoro-benzyl)-2-hydroxy-3-[1-(4-trimethylsilanylethynyl-thiophen-2-yl)-cyclohexylamino]-propyl}-acetamide was stirring in methanol (10 mL) for 30 min. The heterogeneous mixture was filtered and concentrated. The residue was flash chromatographed with 49:1:0.1, 24:1:0.1, and 23:2:0.2 methylene chloride:methanol:concentrated ammonium hydroxide as the eluant to yield N-{1-(3,5-difluoro-benzyl)-3-[1-(4-ethynyl-thiophen-2-yl)-cyclohexylamino]-2-hydroxy-propyl)-acetamide. Method [1] Retention time 1.60 min by HPLC and 1.69 min by MS (M+=447).

### EXAMPLE 248

### PREPARATION OF N-{1-(3,5-DIFLUORO-BENZYL)-3-[1-(4-ETHYL-THIOPHEN-2-YL)-CYCLOHEXYLAMINO]-2-HYDROXY-PROPYL}-ACETAMIDE

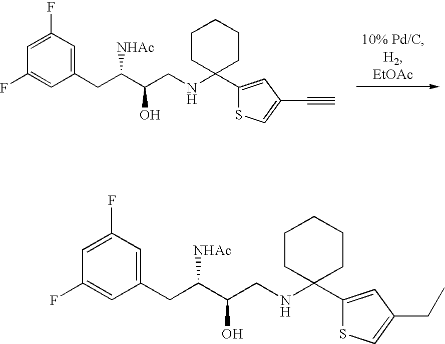

A solution of a mixture of N-{1-(3,5-difluoro-benzyl)-3-[1-(4-ethynyl-thiophen-2-yl)-cyclohexylamino]-2-hydroxy-propyl}-acetamide in ethyl acetate (10 mL) was added to 10% palladium on carbon (50 mg) in a parr bottle. The parr bottle was filled with hydrogen (12 psi) and evacuated three times. The parr bottle was refilled with hydrogen (12 psi) and shook for 1 h. The heterogeneous mixture filtered through celite and concentrated. The residue was flash chromatographed with 49:1:0.1, 24:1:0.1, and 23:2:0.2 methylene chloride:methanol:concentrated ammonium hydroxide as the eluant to yield N-{1-(3,5-difluoro-benzyl)-3-[1-(4-ethyl-thiophen-2-yl)-cyclohexylamino]-2-hydroxy-propyl}-acetamide. Method [1] Retention time 1.75 min by HPLC and 1.84 min by MS (M+=451).

### EXAMPLE 249

### PREPARATION OF N-(1-(3,5-DIFLUORO-BENZYL)-3-{1-[3-(1,1-DIMETHYL-PROPYL)-PHENYL]-CYCLOHEXYLAMINO}-2-HYDROXY-PROPYL)-ACETAMIDE AND N-(1-(3,5-DIFLUORO-BENZYL)-2-HYDROXY-3-{1-[3-(1-METHYL-PROPENYL)-PHENYL]-CYCLOHEXYLAMINO}-PROPYL)-ACETAMIDE

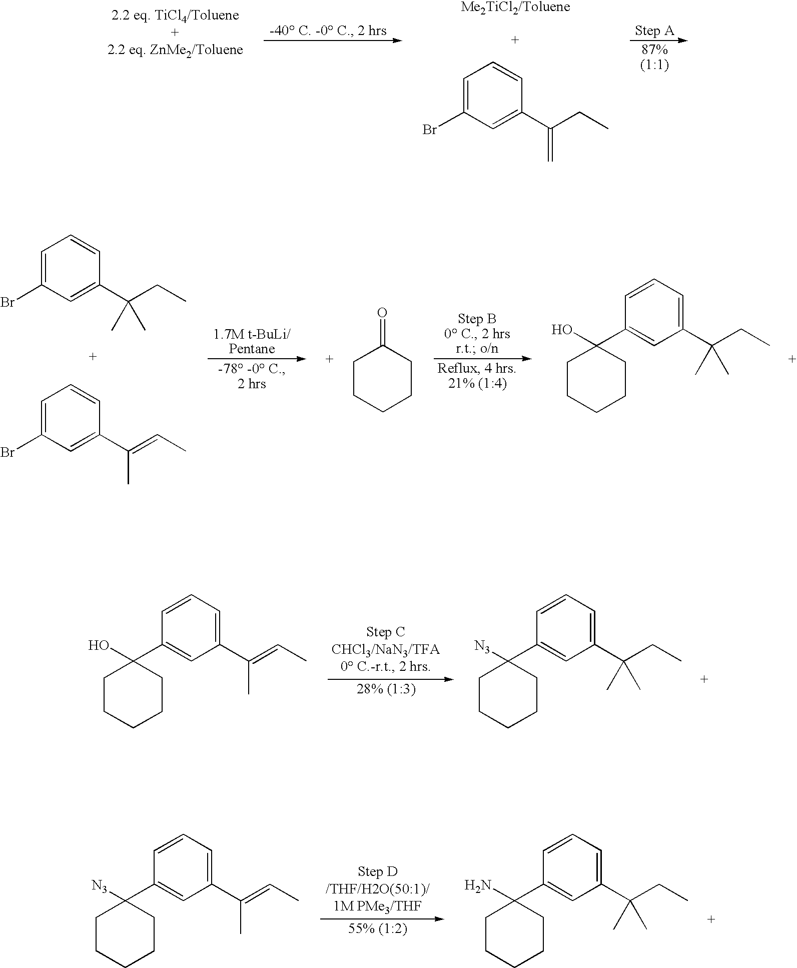

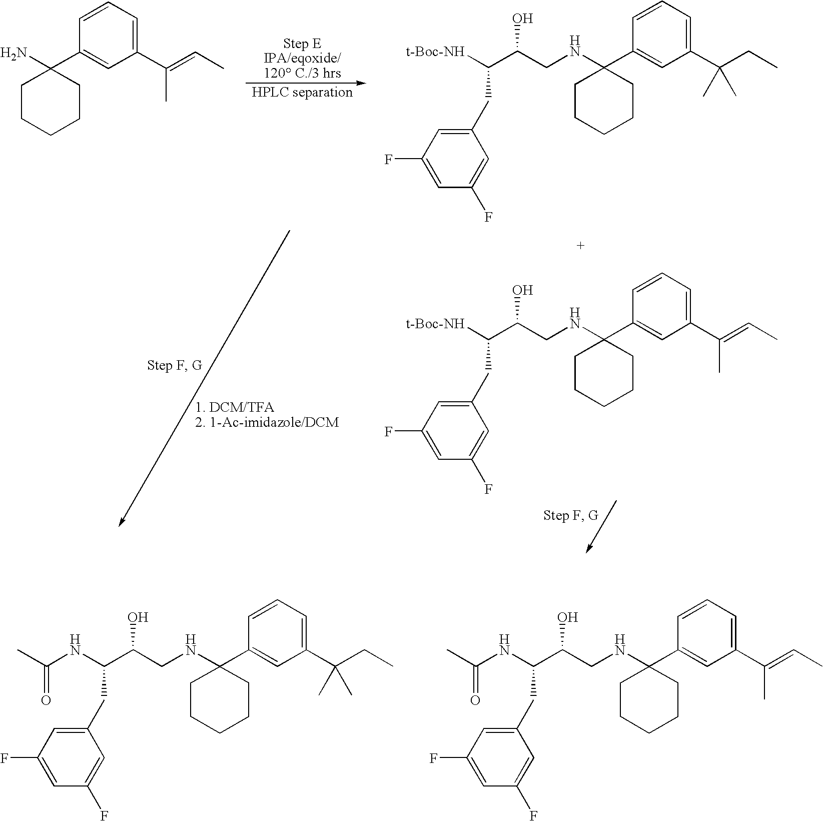

### Step A. 1-Bromo-3-(1,1-dimethyl-propyl)-benzene and 1-Bromo-3-(1-methyl-propenyl)-benzene

To 100 mL (100 mmole, 2.2 eq.) of 1M Titanium tetrachloride/toluene in 100 mL of DCM was cooled to −40° C. and added 50 mL (100 mmole, 2.2 eq.) of 2M dimethylzinc/toluene dropwise and stirred for 30 min., then added 9.6 g (45 mmole) of 3′-bromo-propiophen-one slowly. The reaction stirred for 1 hr and cold bath removed, stirring continued for 3 hrs and monitored by TLC. The reaction was quenched with ice/ether, extracted and washed with water, brine, dried, stripping of solvent gave 8.9 g of 1-Bromo-3-(1,1-dimethyl-propyl)-benzene and 1-Bromo-3-(1-methyl-propenyl)-benzene: Rf (ratio of compound spot/solvent front)=0.84 where the starting material (s. m.) 3′-bromo-propiophenone at Rf=0.43. (10% EtOAc/Hexane). NMR has shown two compounds with ˜1:1 ratio. H NMR (CDCl₃); δ 7.50-7.11 (m, 4H+4H), 5.87-5.83 (q, J=1.1, 6.05, 1.1 Hz, 1H), 1.98 (s, 3H), 1.79-1.76 (dd, J=1.1, 6.05, 1.1 Hz, 3H), 1.64-1.57 (q, J=7.1, 7.7, 7.1 Hz, 2H), 1.25 (s, 6H), 0.69-0.64 (t, J=7.1, 7.7 Hz, 3H). ¹³C NMR (CDCl₃); δ 152.1, 146.2, 134.4, 130.2, 129.8, 129.7, 129.6, 129.3, 129.2, 128.7, 128.5, 124.7, 124.1, 123.9, 122.5, 112.2, 77.4, 77.0, 76.6, 37.9, 36.6, 28.2, 27.8, 15.2, 14.2, 12.7, 8.9.

### EXAMPLE 250

### 1-[3-(1,1-DIMETHYL-PROPYL)-PHENYL]-CYCLOHEXANOL

LCMS m/e=229.1(M−OH), retention time=3.129 min (method [5]).

### EXAMPLE 251

### 1-[3-(1-METHYL-PROPENYL)-PHENYL]-CYCLOHEXANOL

LCMS m/e=213.2(M−OH), retention time=2.736 min (method [5]).

### EXAMPLE 252

### 1-AZIDO-CYCLOHEXYL)-3-(1,1-DIMETHYL-PROPYL)-BENZEN

LCMS m/e=229.1(M−N3), retention time=4.044 min (method [5]).

### EXAMPLE 253

### 1-(1-AZIDO-CYCLOHEXYL)-3-(1-METHYL-PROPENYL)-BENZENE

LCMS m/e=213.2(M−N3), retention time=4.872 min (method [5]).

### EXAMPLE 254

### 1-[3-(1,1-DIMETHYL-PROPYL)-PHENYL]-CYCLOHEXYLAMINE

LCMS m/e=229.1(M−NH2), retention time=2.695 min (method [4]).

### EXAMPLE 255

### 1-[3-(1-METHYL-PROPENYL)-PHENYL]-CYCLOHEXYLAMINE

LCMS m/e=213.2(M−NH2), retention time=2.497 min (method [4]).

### EXAMPLE 256

### 4-(3,5-DIFLUORO-PHENYL)-1-{1-[3-(1,1-DIMETHYL-PROPYL)-PHENYL]-CYCLOHEXYL-AMINO}-3-METHYL-BUTAN-2-OL

LCMS m/e=545.3(M+H), retention time=3.242 min (method [4]).

### EXAMPLE 257

### 4-(3,5-DIFLUORO-PHENYL)-3-METHYL-1-{1-[3-(1-METHYL-PROPENYL)-PHENYL]-CYCLOHEXYLAMINO}-BUTAN-2-OL

LCMS m/e=529.3(M+H), retention time=3.052 min (method [4]).

### EXAMPLE 258

### N-(1-(3,5-DIFLUORO-BENZYL)-3-{1-[3-(1,1-DIMETHYL-PROPYL)-PHENYL]-CYCLOHEXYLAMINO}-2-HYDROXY-PROPYL)-ACETAMIDE

LCMS m/e=487.3(M+H), retention time=2.776 (method [4]) 1H NMR (CDCl3); δ 7.55 (s, 1H), 7.39-7.33 (m, 3H), 6.70-6.63 (m, 2H), 6.25-6.22 (d, J=8.8 Hz, 1H), 4.05 (m, 1H), 3.71 (m, 1H), 2.99-2.95 (m, 2H), 2.77-2.46 (m, 3H), 2.03 (m, 2H), 1.84 (s, 3H), 1.79-1.77 (m, 2H), 1.68-1.60 (m, 6H), 1.45-1.36 (m, 4H), 1.29 (s, 6H), 0.66-0.61 (t, J=7.7, 7.7 Hz, 3H).

### EXAMPLE 259

### N-(1-(3,5-DIFLUORO-BENZYL)-2-HYDROXY-3-{1-[3-(1-METHYL-PROPENYL)-PHENYL]-CYCLOHEXYLAMINO}-PROPYL)-ACETAMIDE

LCMS m/e=471.2(M+H), retention time=2.609 (method [4]): 1H NMR (CDCl3); δ 7.78-7.37 (m, 4H), 6.69-6.47 (m, 3H), 5.92-5.90 (q, J=1.7, 6.2, 1.7 Hz, 1H), 4.01 (m, 1H), 3.76-3.74 (m, 1H), 2.99-2.95 (m, 2H), 2.74-2.62 (m, 6H), 2.44 (m, 1H), 2.03 (s, 3H), 1.81 (s, 3H), 1.80 (s, 3H), 1.60 (m, 2H), 1.37-1.08 (m, 6H).

### EXAMPLE 260

### PREPARATION OF N-[3-{1-[3-(CYANO-DIMETHYL-METHYL)-PHENYL]-CYCLOHEXYLAMINO}-1-(3,5-DIFLUORO-BENZYL)-2-HYDROXY-PROPYL]-ACETAMIDE

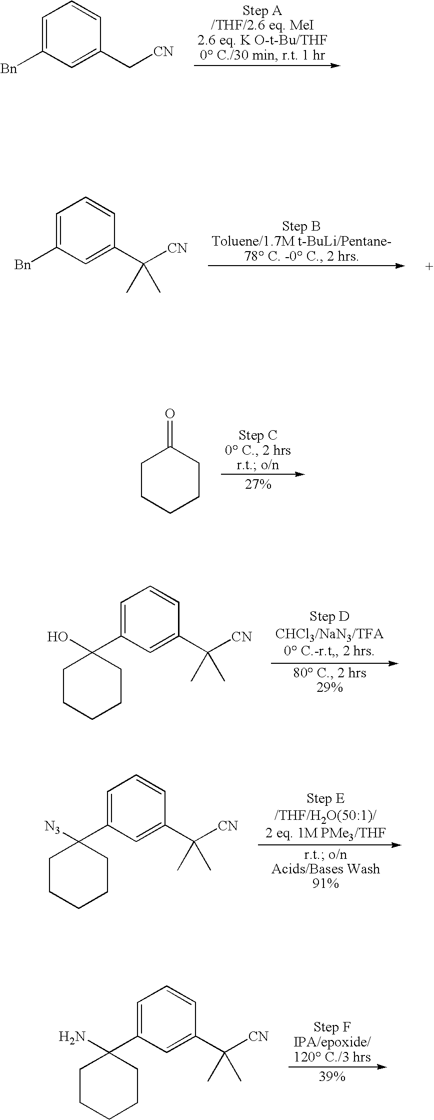

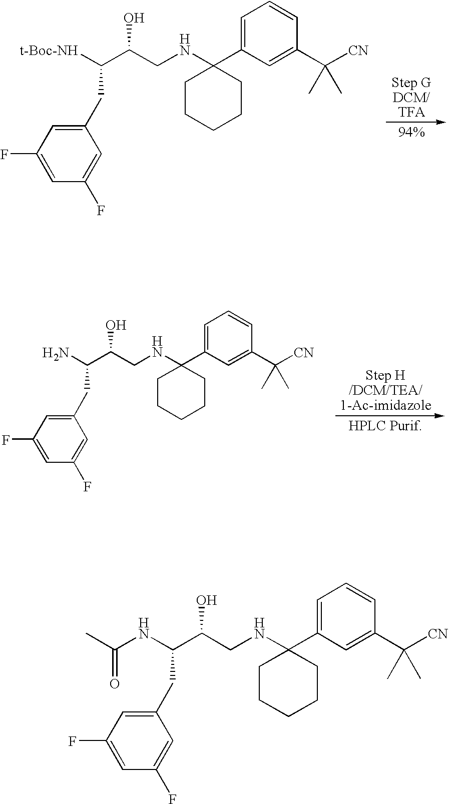

### Step A. 2-(3-Bromo-phenyl)-2-methyl-propionitrile

To 7.84 g (40 mmole) of m-bromo-phenyl acetonitrile in 20 mL of THF and 14.75 g (104 mmole, 2.6 eq.) of iodomethane was cooled to -78° C. and added 104 mL (104 mmole, 2.6 eq.) of 1 M potassium t-butoxide dropwise for 30 min., the reaction stirred for 1 hr and cold bath removed, another 1.6 eq. of iodomethane was added and stirring continued for another 2 hrs and monitored by TLC. The reaction was quenched with ice/ether, extracted and washed with water, brine, dried, stripping of solvent gave 8.46 g (37.78 mmole, 94%) of 2-(3-Bromo-phenyl)-2-methyl-propionitrile, TLC: Rf=0.59 where s. m. at Rf=0.43. (10% EtOAc/Hexane). IR CN peak @2237.5 cm⁻¹. ¹H NMR (CDC13); δ 7.61-7.60 (m, 1H), 7.47-7.41 (m, 2H), 7.30-7.27 (m, 1H), 1.72 (s, 6H). ¹³C NMR (CDCl3); δ 143.7, 131.1, 130.6, 128.3, 123.9, 123.1, 77.4, 77.0, 76.6, 36.8, 28.9.

### EXAMPLE 261

### 2-[3-(1-HYDROXY-CYCLOHEXYL)-PHENYL]-2-METHYL-PROPIONITRILE

LCMS m/e=226.1(M−OH), retention time=1.128 min (method [2]).

### EXAMPLE 262

### 2-[3-(1-AZIDO-CYCLOHEXYL)-PHENYL]-2-METHYL-PROPIONITRILE

LCMS m/e=226.1(M−N3), retention time=2.104 min (method [1]).

### EXAMPLE 263

### 2-[3-(1-AMINO-CYCLOHEXYL)-PHENYL]-2-METHYL-PROPIONITRILE

LCMS m/e=226.1 (M+H), retention time=0.255 min (method [2]).

### EXAMPLE 264

### 2-(3-{1-[4-(3,5-DIFLUORO-PHENYL)-2-HYDROXY-3-METHYL-BUTYLAMINO]-CYCLOHEXYL}-PHENYL)-2-METHYL-PROPIONITRILE

LCMS m/e=542.3(M+H), retention time=1.976 min (method [3]).

### EXAMPLE 265

### 2-(3-{1-[3-AMINO-4-(3,5-DIFLUORO-PHENYL)-2-HYDROXY-BUTYLAMINO]-CYCLOHEXYL}-PHENYL)-2-METHYL-PROPIONITRILE

LCMS m/e=442.2(M+H), retention time=1.453 min (method [1]).

### EXAMPLE 266

### N-[3-{1-[3-(CyANO-DIMETHYL-METHYL)-PHENYL]-CYCLOHEXYLAMINO}-1-(3,5-DIFLUORO-BENZYL)-2-HYDROXY-PROPYL]-ACETAMIDE

LCMS m/e=484.2(M+H), retention time=1.713 min (method [1]). 1H NMR (CDCl3); δ 7.74-7.27 (m, 4H), 6.70-6.51 (m, 3H), 4.03 (q, 1H), 3.71 (m, 1H), 2.99-2.96 (d, J=0.4 Hz, 2H), 2.73-2.49 (m, 5H), 2.04 (m, 2H), 1.84 (s, 3H), 1.74 (s, 6H). 1.57-1.215 (m, 8H). ¹³C NMR (CDCl3); δ 143.1, 129.9, 127.3, 126.1, 124.6, 111.9, 102.3, 100.1, 77.4, 77.0, 7.6, 69.2, 63.4, 52.4, 44.8, 35.3, 33.8, 32.9, 28.8, 25.4, 24.8, 22.8, 21.8, 17.9.

### EXAMPLE 267

### PREPARATION OF N-{1-(3,5-DIFLUORO-BENZYL)-3-[1-(3-DIMETHYLAMINO-PHENYL)-CYCLOHEXYLAMINO]-2-HYDROXY-PROPYL}-ACETAMIDE

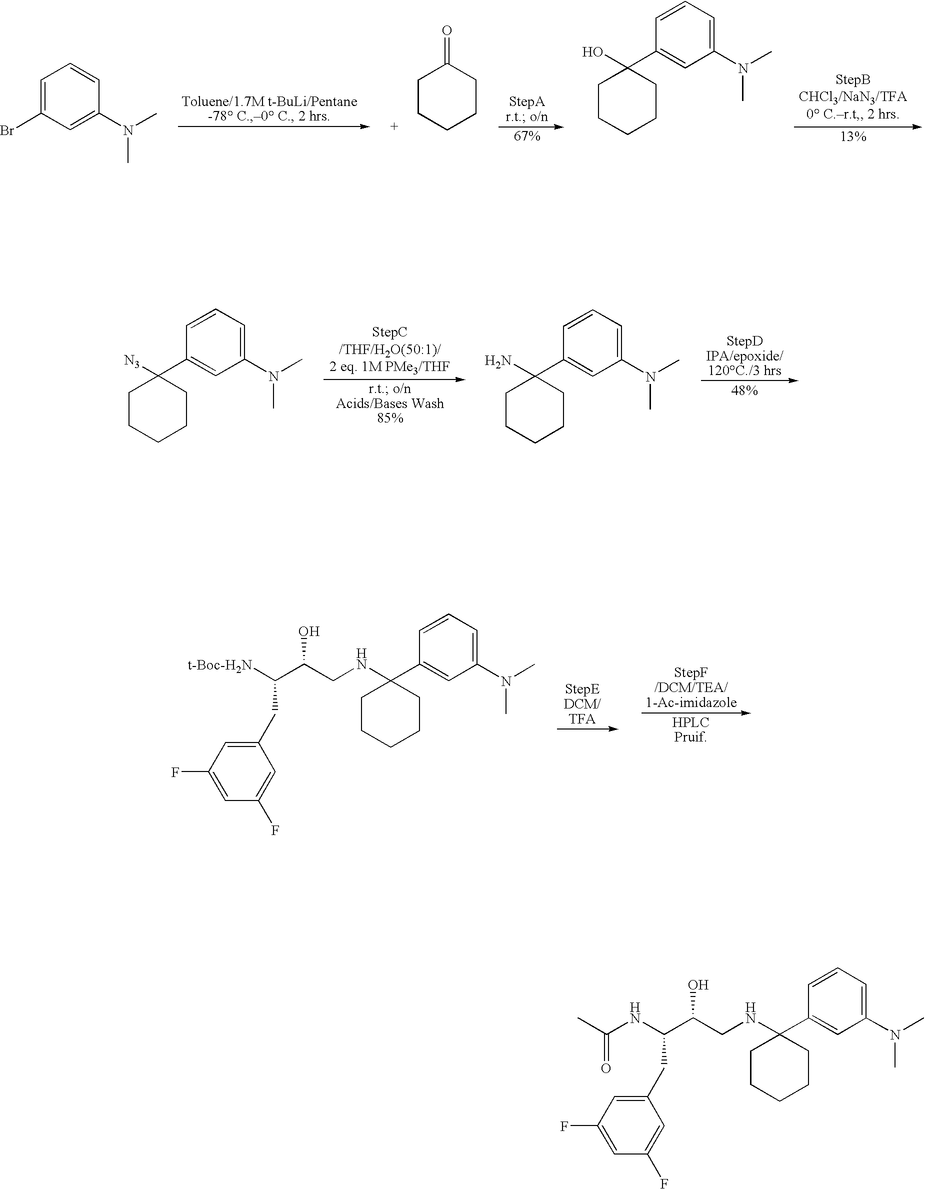

### EXAMPLE 268

### 1-(3-DIMETHYLAMINO-PHENYL)-CYCLOHEXANOL

LCMS m/e=202.6(M−OH), retention time=0.836 min (method [2]).

### EXAMPLE 269

### [3-(1-AZIDO-CYCLOHEXYL)-PHENYL]-DIMETHYL-AMINE

LCMS m/e=201.8(M−N3), retention time=0.501 min (method [2]).

### EXAMPLE 270

### [3-(1-AMINO-CYCLOHEXYL)-PHENYL]-DIMETHYL-AMINE

LCMS m/e=219.7(M+H), retention time=0.261 min (method [2]).

### EXAMPLE 271

### 4-(3,5-DIFLUORO-PHENYL)-1-[1-(3-DIMETHYLAMINO-PHENYL)-CYCLOHEXYLAMINO]-3-METHYL-BUTAN-2-OL

LCMS m/e=517.9(M+H), retention time=1.880 min (method [1]).

### EXAMPLE 272

### N-{1-(3,5-DIFLUORO-BENZYL)-3-[1-(3-DIMETHYLAMINO-PHENYL)-CYCLOHEXYLAMINO]-2-HYDROXY-PROPYL)-ACETAMIDE

LCMS m/e=496.2(M+Na), retention time=2.039 min (method [4]). 1H NMR (CDCl₃); δ 7.71 (s, 1H), 7.55-7.49 (t, J=8.2, 7.7 Hz, 1H), 7.39-7.31 (dd, J=7.7, 9.9, 8.3 Hz, 2H), 7.17-7.14 (d, J=8.2 Hz, 1H), 6.69-6.59 (m, 3H), 4.03 (m, 1H), 3.74 (m, 1H), 3.15 (s, 6H), 3.01-2.95 (m, 1H), 2.91-2.57 (m, 6H), 2.07-1.93 (m, 3H), 1.81 (s, 3H), 1.59 (m, 2H), 1.38-1.35 (m, 4H). ¹³C NMR (CDCl3); δ 178.1, 162.3, 161.5, 137.0, 131.1, 125.4, 118.8, 117.7, 112.1, 111.7, 77.4, 77.0, 76.6, 69.2, 64.1, 52.8, 44.5, 44.2, 35.6, 33.3, 32.4, 24.7, 22.3, 21.8.

### EXAMPLE 273

### PREPARATION OF N-{1-(3,5-DIFLUORO-BENZYL)-2-HYDROXY-3-[1-(3-METHANESULFONYL-PHENYL)-CYCLOHEXYLAMINO]-PROPYL}-ACETAMIDE

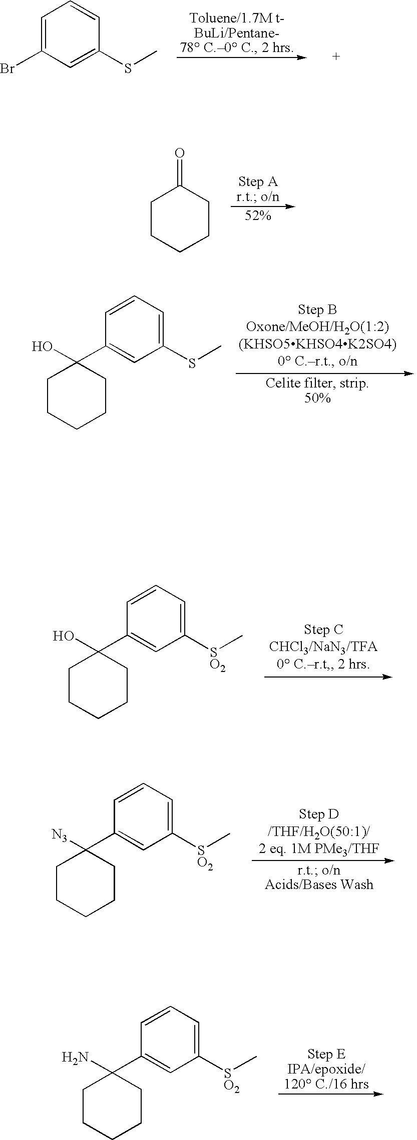

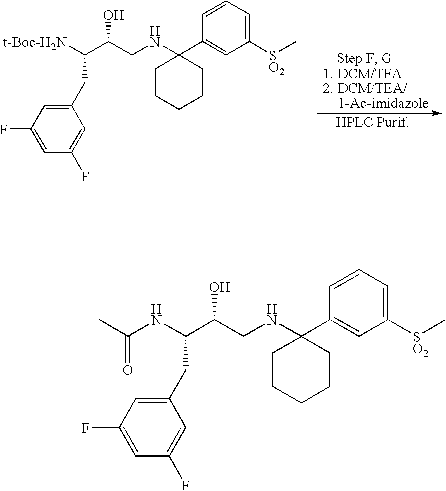

### Step A. 1-(3-Methanesulfonyl-phenyl)-cyclohexanol (LF3786-37A)

To 11.9 g (19.37 mmole, 2.5 eq.) of oxone in a mixture of 30 mL of methanol and 60 mL of water was added 1.72 g (7.75 mmole) of LF3786-36A in 30 mL of methanol slowly at 0° C. Cold bath was then removed and stirring continued for another 4 hrs and monitored by TLC. The reaction mixture filtered through celite, and partirion between DCM and water, The solvent was removed in vacuo and the residue was purified by flash column to yield 0.99 g of LF3786-37A as a beige solid. (50%). TLC (30% EtOAc/Hexane) Rf=0.20. LCMS m/e=237.0(M−OH), retention time=0.484 min (method [2])

### EXAMPLE 274

### 1-(3-METHYLSULFANYL-PHENYL)-CYCLOHEXANOL

LCMS m/e=205.1 (M−OH), retention time=1.270 min (method [2]).

### EXAMPLE 275

### 1-(1-AZIDO-CYCLOHEXYL)-3-METHANESULFONYL-BENZENE

LCMS m/e=237.1(M−N₃), retention time=2.260 min (method [1]).

### EXAMPLE 276

### 1-(3-METHANESULFONYL-PHENYL)-CYCLOHEXYLAMINE

LCMS m/e=237.1(M−NH₂), retention time=0.240 min (method [2]).

### EXAMPLE 277

### 4-(3,5-DIFLUORO-PHENYL)-1-[1-(3-METHANESULFONYL-PHENYL)-CYCLOHEXYLAMINO]-3-METHYL-BUTAN-2-OL

LCMS m/e=553.2(M+H), retention time=1.795 min (method [1]).

### EXAMPLE 278

### N-{1-(3,5-DIFLUORO-BENZYL)-2-HYDROXY-3-(1-(3-METHANESULFONYL-PHENYL)-CYCLOHEXYLAMINO]-PROPYL}-ACETAMIDE

LCMS m/e=517.1(M+Na), retention time=0.261 min (method [1]). ¹H NMR (CD₃OD); δ 8.23 (s, 1H), 8.11-8.08 (d, J=7.7 Hz, 1H), 8.03-8.01 (d, J=7.7 Hz, 1H), 7.86-7.80 (t, J=7.7, 8.2 Hz, 1H), 6.82-6.76 (m, 3H), 3.87-3.84 (m, 1H), 3.63-3.57 (m, 1H), 3.32-3.31 (t, J=2.8, 1.6 Hz, 1H), 2.69 (s, 3H), 2.59-2.50 (m, 1H), 2.07-1.82 (m, 3H), 1.79 (s, 3H), 1.64 (m, 2H), 1.47-1.29 (m, 7H). ¹³C NMR (CD₃OD); δ 174.7, 143.8, 134.9, 132.1, 129.7, 128.2, 113.1, 112.8, 102.8, 70.4, 65.0, 61.3, 54.6, 49.9, 49.6, 49.3, 49.0, 48.9, 48.7, 48.4, 48.1, 46.1, 44.2, 36.7, 34.7, 33.2, 25.9, 23.0, 22.9, 22.3.

### EXAMPLE 279

### PREPARATION OF N-[3-{1-[3-(2,2-DICHLORO-1-METHYL-CYCLOPROPYL)-PHENYL]-CYCLOHEXYLAMINO}-1-(3,5-DIFLUORO-BENZYL)-2-HYDROXY-PROPYL]-ACETAMIDE

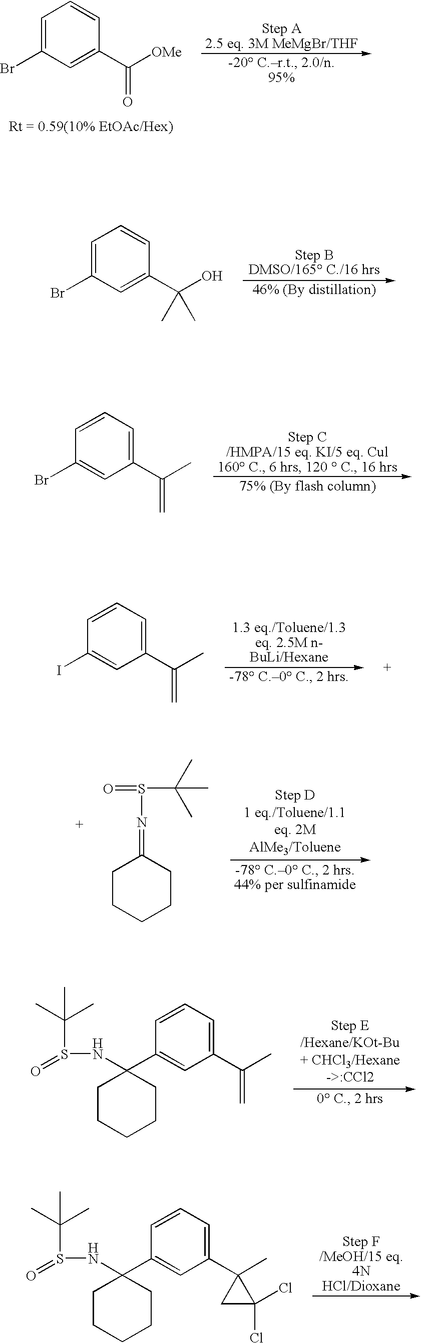

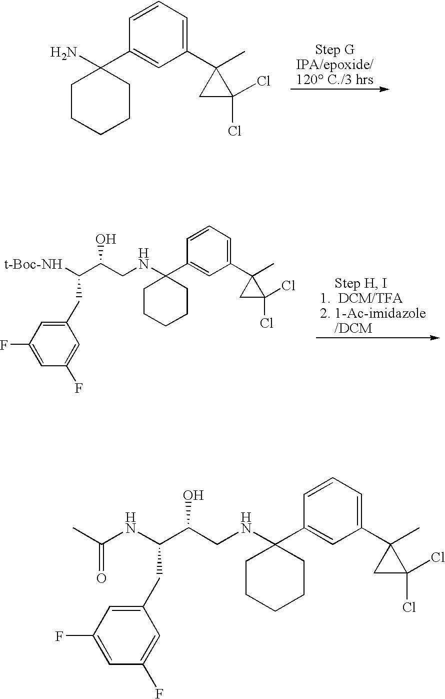

### Step A. 2-(3-Bromo-phenyl)-propan-2-ol

To 15 g (71.6 mmole) of Methyl 3-bromobenzoate in 50 mL of THF was added 60 mL (179 mmole, 2.5 eq.) of 3M MeMgBr/ether dropwise at 0° C., The reaction stirred for 1 hr and cold bath removed, stirring continued overnight and monitored by TLC. The reaction was quenched with ice/ether, extracted and washed with water, brine, dried, stripping of solvent gave 15.1 g of 2-(3-Bromo-phenyl)-propan-2-ol (95% yield). TLC (10% EtOAc/Hexane): Rf=0.36 where s. m. at Rf=0.59.

### Step B. 1-Bromo-3-isopropenyl-benzene

To 15.1 g (70 mmole) of LF3786-48A in 30 mL of DMSO was heated at 165° C. overnight. Replaced by a short-stemed distillation aparatus and ditilled with an oil bath under vacuum yielding 6.33 g of Bromo-3-isopropenyl-benzene as a clear liquid (46% yield). TLC (10% EtOAc/Hexane): Rf=0.79 where s. m. at Rf=0.41.

### Step C. 1-Iodo-3-isopropenyl-benzene

To 3.94 g (20 mmole) of 1-Bromo-3-isopropenyl-benzene in 40 mL of HMPA and 57.1 g (300 mmole, 15 eq.) of potassium iodide and 16.6 g (100 mmole, 5 eq.) of copper (I) iodide was heated to 160° C for 6 hrs (50% done), followed by 120° C. for 16 hrs. After cooling, filtered solid and partition between ether and water, Flash column gave 3.68 g of 1-Iodo-3-isopropenyl-benzene (75% yield).

### Step D. 2-Methyl-propane-2-sulfinic acid [1-(3-isopropenyl-phenyl)-cyclohexyl]-amide.

Used two 25 mL round bottom flasks (RBF) from oven and cooled them to room temperature. To a stirred solution of 1-Iodo-3-isopropenyl-benzene (3.33 g, 13.6 mmol., 2.1 eq.) in 20 mL of toluene (dry) at −78° C. under nitrogen was added 2.5 M n-butyl lithium/hexane (new bottle)(5.5 mL, 13.6 mmol., 2.1 eq.). The reaction was stirred for 1 h warming to room termperature. The reaction was cooled back down to −78° C., in a separate RBF the t-Bu sulfonimine (2.11 g, 10.5 mmol, 1.0 eq.) was dissolved in 10 mL toluene (dry) at −78° C. under nitrogen and 2 M trimethylaluminum/toluene (5.8 mL, 11.55 mmol., 1.1 eq.) was added dropwise by syringe. After 5 min the sulfonimine/AIMe₃ solution was cannulated into the iodo. t-butyl/butyl lithium solution. The reaction was stirred for 2 hr warming to room temperature. The reaction was quenched with sodium sulfate decahydrate until the bubbling stopped. magnesium sulfate was added to the reaction and stirred for 30 min. The reaction was filtered, rinsed with EtOAc and concentrated down. Ran a column eluting with 30% EtOAc/Hexanes yielding 1.46 g of 2-Methyl-propane-2-sulfinic acid [1-(3-isopropenyl-phenyl)-cyclohexyl]-amide as a white solid (44% yield per sulfimamide). LCMS m/e=320.2(M+H), retention time=2.690 min (method [2]):

### Step E. 2-Methyl-propane-2-sulfinic acid {1-[3-(2,2-dichloro-1-methyl-cyclopropyl)-phenyl]-cyclohexyl}-amide

To 0.31 mL (3.72 mmole, 2.5 eq.) of chloroform in 2 mL of hexane was added dropwise a mixture of 0.48 g (1.49 mmole) of 2-Methyl-propane-2-sulfinic acid [1-(3-isopropenyl-phenyl)-cyclohexyl]-amide in 2 mL of heaxane and 4.5 mL (4.47 mmole, 3 eq.) of 1 M potassium tert-butoxide in THF at 0° C. The reaction stirred for 1 hr and cold bath removed, stirring continued overnight and monitored by HPLC/MS.

Additional 0.31 mL (3.72 mmole, 2.5 eq.) of chloroform in 2 mL of hexane and 4.5 mL (4.47 mmole, 3 eq.) of 1 M potassium tert-butoxide in THF was needed to facilitate the reaction. The reaction was quenched with ice/EtOAc, extracted and washed with water, brine, dried, stripping of solvent, flash column gave 0.14 g of 2-Methyl-propane-2-sulfinic acid {1-[3-(2,2-dichloro-1-methyl-cyclopropyl)-phenyl]-cyclohexyl}-amide. (23% yield). TLC (50% EtOAc/Hexane): Rf=0.36 where s. m. at Rf=0.32. LCMS m/e=402.1(M+H), retention time=2.963 min (method [1]).

### Step F. 1-[3-(2,2-Dichloro-1-methyl-cyclopropyl)-phenyl]-cyclohexylamine

To 0.14 g (0.35 mmole) of 2-Methyl-propane-2-sulfinic acid {1-[3-(2,2-dichloro-1-methyl-cyclopropyl)-phenyl]-cyclohexyl}-amide in 5 mL of methanol was added 1.31 mL (5.23 mmole, 15 eq.) of 4N HCl in dioxane at r.t. The reaction was stirred for 2 hrs and monitored by TLC. The solvent was stripped off yielding 72 mg of 1-[3-(2,2-Dichloro-1-methyl-cyclopropyl)-phenyl]-cyclohexylamine (69%). LCMS m/e=281.0(M−NH₂), retention time=1.639 min (method [1]).

### EXAMPLE 280

### 1-{1-[3-(2,2-DICHLORO-1-METHYL-CYCLOPROPYL)-PHENYL]-CYCLOHEXYLAMINO}-4-(3,5-DIFLUORO-PHENYL)-3-METHYL-BUTAN-2-OL

LCMS m/e=597.1(M+H), retention time=2.316 min (method [1]).

### EXAMPLE 281

### N-[3-{1-[3-(2,2-DICHLORO-1-METHYL-CYCLOPROPYL)-PHENYL]-CYCLOHEXYLAMINO}-1-(3,5-DIFLUORO-BENZYL)-2-HYDROXY-PROPYL]-ACETAMIDE

LCMS m/e=539.1(M+H), retention time=1.988 min (method [1]). ¹H NMR (CDCl3); δ 7.54-7.39 (m, 4H), 6.69-6.64 (m, 3H), 4.02 (m, 1H), 3.79-3.72 (m, 1H), 3.31 (m, 2H), 2.93 (m, 1H), 2.73-2.59 (m, 4H), 1.85 (s, 3H), 1.69-1.68 (ds, 3H), 1.66-1.62 (m, 4H), 1.41-1.33 (m, 2H), 1.31-1.30 (ds, 2H).

### EXAMPLE 282

### N-{1-(3,5-DIFLUORO-BENZYL)-2-HYDROXY-3-[1-(3-ISOPROPENYL-PHENYL)-CYCLOHEXYLAMINO]-PROPYL}-ACETAMIDE

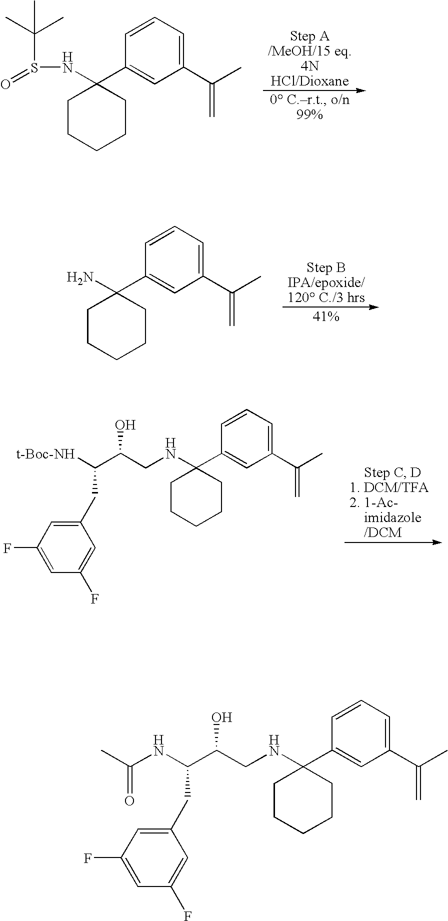

### EXAMPLE 283

### 1-(3-ISOPROPENYL-PHENYL)-CYCLOHEXYLAMINE

LCMS m/e=199.1(M−NH₂), retention time=1.418 min (method [2]).

### EXAMPLE 284

### 4-(3,5-DIFLUORO-PHENYL)-1-[1-(3-ISOPROPENYL-PHENYL)-CYCLOHEXYLAMINO]-3-METHYL-BUTAN-2-OL

LCMS m/e=515.2(M+H), retention time=2.188 min (method [1]).

### EXAMPLE 285

### N-{1-(3,5-DIFLUORO-BENZYL)-2-HYDROXY-3-[1-(3-ISOPROPENYL-PHENYL)-CYCLOHEXYL AMINO]-PROPYL}-ACETAMIDE

LCMS m/e=457.2(M+H), retention time=1.832 min (method [1]). ¹H NMR (CDCl3); δ 8.06-7.43 (m, 4H), 6.69-6.13 (m, 3H), 5.43-5.16 (ds, J=80.2 Hz, 2H), 4.05 (m, 1H), 3.69 (m, 1H), 3.02 (m, 2H), 2.65-2.48 (m, 9H), 2.16 (s, 3H), 1.86 (m, 2H), 1.83 (s, 3H), 1.59-1.30 (m, 4H).

### EXAMPLE 286

### PREPARATION OF 4-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-BUTYRIC ACID METHYL ESTER

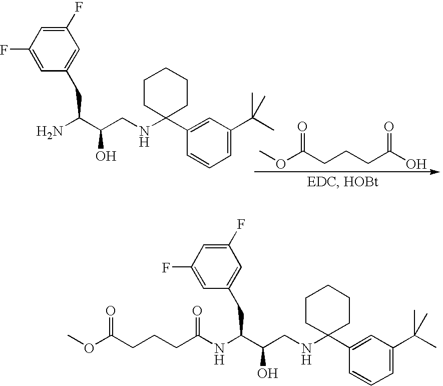

3-Amino-1-[1-(3-tert-butyl-phenyl)-cyclohexylamino]-4-S-(3,5-difluoro-phenyl)-R-butan-2-ol (1.16 mmol, 0.50 g) was charged to a 50 mL, flame dried round bottom flask and taken up in CH₂Cl₂ (10 mL). Pentanedioic acid monomethyl ester (1.16 mmol, 0.17 g) was added, followed by N-methylmorpholine (3.5 mmol, 354 mg, 0.38 mL), 1-Hydroxybenzotriazole (HOBt, 1.4 mmol, 189 mg), and 1-(3-dimethylaminopropyl)-3-ethylcarbodiimide hydrochloride (EDC, 1.4 mmol, 267 mg). The homogenous reaction was stirred at 23° C. for 16 h under nitrogen before being quenched by the addition of an aqueous, saturated solution of ammonium chloride. The mixture was extracted with ethyl acetate (2×50 mL) and the organic solution was rinsed with aqueous, saturated solutions of sodium bicarbonate and sodium chloride (50 mL each) in sequence. The ethyl acetate solution was dried over sodium sulfate and concentrated under reduced pressure to yield a yellow oil as the crude product. The crude product was purified by column chromatography using a mobile phase of 7% MeOH/methylene chloride yielding 4-[3-[1-(3-tert-Butyl-phenyl)-cyclohexylamino]-1-S-(3,5-difluoro-benzyl)-R-2-hydroxy-propylcarbamoyl]-butyric acid methyl ester as a pure, colorless oil. ¹H NMR (300 MHz, CDCl₃); δ 7.48 (s, 1H), 7.32-7.20 (m, 3H), 6.66-6.51 (m, 3H), 4.15-4.10 (m, 1H), 3.66 s, 3H), 2.74-2.65 (m, 1H), 2.78-2.60 (m, 2H), 2.25-2.10 (m, 4H), 1.92-1.87 (m, 6H), 1.62-1.50 (m, 6H), 1.34 (s, 9H); ¹³C NMR (75 MHz, CDCl₃); δ 173.5, 172.0, 164.4 (d, J=12.9 Hz), 161.1 (d, J=12.9 Hz), 150.8, 145.6, 142.2 (t, J=9.2 Hz), 127.7, 123.2 (d, J=8.5 Hz), 111.8 (m), 101.7 (t, J=24.8 Hz), 70.7, 57.0, 53.0, 51.4, 43.1, 36.4, 36.2, 36.0, 35.4, 34.7, 32.7, 31.3, 25.8, 22.1, 22.0, 20.8.

### EXAMPLE 287

### PREPARATION OF 4-[3-[1-(3-TERT-BUTYL-PHENYL)-CYCLOHEXYLAMINO]-1-S(3,5-DIFLUORO-BENZYL)-R-2-HYDROXY-PROPYLCARBAMOYL]-BUTYRIC ACID

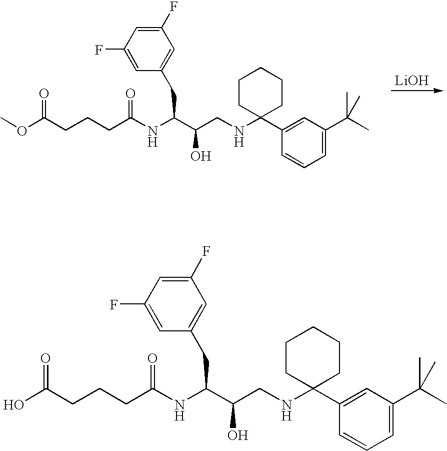

To a THF (3 mL) solution of 4-[3-[1-(3-tert-Butyl-phenyl)-cyclohexylamino]-1-S-(3,5-difluoro-benzyl)-R-2-hydroxy-propylcarbamoyl]-butyric acid methyl ester (0.43 mmol, 204 mg) was added MeOH (1 mL), water (1 mL), and LiOH (2 mmol, 90 mg). The reaction was allowed to stir for 16 h at 23° C before being diluted with 10% aqueous sodium carbonate. The aqueous solution was rinsed with ethyl ether (30 mL) before being brought to pH 3 be addition of 1 N HCl. The acidic solution was extracted with ethyl acetate (30 mL) and methylene chloride (30 mL) and the extracts were combined, dried over sodium sulfate and concentrated under reduced pressure yielding a fluffy white solid as 4-[3-[1-(3-tert-Butyl-phenyl)-cyclohexylamino]-1-S-(3,5-difluoro-benzyl)-R-2-hydroxy-propylcarbamoyl]-butyric acid. ¹H NMR (300 MHz, CD₃OD); δ 7.69 (s, 1H), 7.55-7.45 (m, 3H), 6.85-6.74 (m, 3H), 4.11 (m, 1H), 3.91 (m, 1H), 3.00 (dd, J=15.12 Hz), 2.57 (m, 4H), 2.39 (dd, J=12.8 Hz), 2.12-2.00 (m, 4H), 1.85-1.65 (m, 10H), 1.44 (s, 9H); ¹³C NMR (75 MHz, CD₃OD); δ 177.2, 174.2, 164.3 (d, J=12.6 Hz), 161.0 (d, J=12.6 Hz), 152.4, 142.2 (t, J=9.2 Hz), 134.0, 128.9, 126.0, 124.5, 124.4, 111.9, 111.6, 103.8, 101.6 (t, J=25.3 Hz), 68.9, 63.8, 52.7, 44.8, 35.4, 35.1, 34.8, 34.3, 33.3, 33.1, 31.0, 29.5, 24.9, 21.9, 21.2.

### EXAMPLE 288

### PREPARATION OF PENTANEDIOIC ACID [3-[1-(3-TERT-BUTYL-PHENYL)-CYCLOHEXYLAMINO]-1-S(3,5-DIFLUORO-BENZYL)-R-2-HYDROXY-PROPYL]-AMIDE METHYLAMIDE

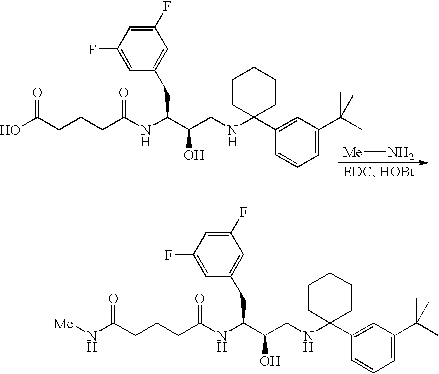

An oven dried, 20 mL vial was charged with 4-[3-[1-(3-tert-Butyl-phenyl)-cyclohexylamino]-1-S-(3,5-difluoro-benzyl)-R-2-hydroxy-propylcarbamoyl]-butyric acid (0.1 mmol, 55 mg) as a white powder. CH₂Cl₂ (1 mL) was added, followed by methyl amine (0.2 mmol, 0.1 mL of a 2M solution in THF), N-methylmorpholine (0.2 mmol, 20 mg), HOBt (0.12 mmol, 17 mg) and EDC (0.12 mmol, 23 mg). The reaction was stirred under nitrogen for 16h at 23° C. before being quenched by the addition of an aqueous, saturated solution of ammonium chloride (10 mL). The resulting mixture was extracted with ethyl acetate (2×20 mL). The extracts were combined and rinsed with an aqueous, saturated solution of sodium bicarbonate before being dried over sodium sulfate and concentrated under reduced pressure yielding the crude product as an oil. Purification was by HPLC using a C18 column yielding pure pentanedioic acid [3-[1-(3-tert-butyl-phenyl)-cyclohexylamino]-1-S-(3,5-difluoro-benzyl)-R-2-hydroxy-propyl]-amide methylamide (retention time=1.85, method [1]). ¹H NMR (300 MHz, CDCl₃); δ 9.12 (bs, 1H), 8.23 (bs, 1H), 7.64 (s, 1H), 7.46-7.33 (m, 3H), 6.71-6.60 (m, 3H), 6.38 (bs, 1H), 4.08 (m, 1H), 3.75 (m, 1H), 2.96 (dd, J=14.4, 3.9 Hz, 1H), 2.72 (s, 3H), 2.65-2.53 (m, 5H), 2.15-1.98 (m, 6H), 1.79-1.76 (m, 4H), 1.34 (s, 9H); ¹³C NMR (75 MHz, CD₃OD); δ 174.2, 173.7, 164.3 (d, J=12.7 Hz), 161.2 (d, J=12.7 Hz), 152.7, 141.7 (t, J=9.2 Hz), 133.9, 128.9, 126.2, 124.6, 112.1, 111.7, 102.0, 69.4, 64.4, 52.7, 44.5, 35.1, 35.0, 34.9, 34.4, 33.5, 32.9, 31.1, 26.2,24.9, 21.9, 21.5.

### EXAMPLE 289

### PREPARATION OF PENTANEDIOICAMIDE-[3-[1-(3-TERT-BUTYL-PHENYL)-CYCLOHEXYLAMINO]-1-S(3,5-DIFLUORO-BENZYL)-2-R-HYDROXY-PROPYL]-AMIDE

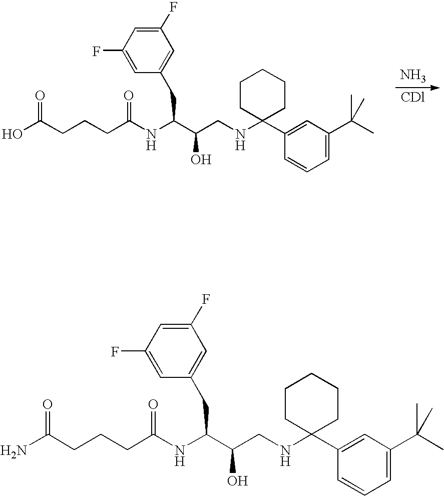

An oven dried, 20 mL vial was charged with 4-[3-[1-(3-tert-Butyl-phenyl)-cyclohexylamino]-1-S-(3,5-difluoro-benzyl)-R-2-hydroxy-propylcarbamoyl]-butyric acid (0.1 mmol, 55 mg) as a white powder. Methylene chloride (4 mL) was added, and the solution was chilled to 0° C. before 1,1′-carbonyldiimidazole (CDI, 0.42 mmol, 19 mg) was added as a solid and stirred for 0.5 h. Gaseous ammonia was bubbled through the solution for 10 min. The solution was warmed to 23° C. and stirred for 16 h before being concentrated under reduced pressure and purified by HPLC using a C18 column yielding pure Pentanedioicamide-[3-[1-(3-tert-butyl-phenyl)-cyclohexylamino]-1-S-(3,5-difluoro-benzyl)-2-R-hydroxy-propyl]-amide as the trifluoroacetate salt: ¹H NMR (300 MHz, CD₃OD); δ 7.56 (s, 1H), 7.42-7.28 (m, 3H), 6.70-6.54 (m, 3H), 3.88 (m, 1H), 3.58 (m, 1H), 3.24 (dd, J=14.3 Hz), 2.60-2.52 (m, 4H), 2.44 (dd, J=12.3 Hz), 2.15-2.07 (m, 4H), 1.98-1.72 (m, 10H), 1.35-1.28 (m, 15H); ¹³C NMR (75 MHz, CD₃OD); δ 178.9, 174.6, 164.3 (d, J=12.5 Hz), 161.0 (d, J=12.5 Hz), 152.3, 142.2 (t, J=9.2 Hz), 134.7, 128.7, 125.8, 124.4, 124.3, 121.2, 111.8, 111.7, 111.5, 101.6 (t, J=25.3 Hz), 69.0, 63.2, 52.7, 44.5, 35.6, 35.4, 35.2, 34.7, 33.6, 33.1, 30.9, 24.9, 21.8, 21.7.

### EXAMPLE 290

### PREPARATION OF 5-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-PENTANOIC ACID METHYL ESTER

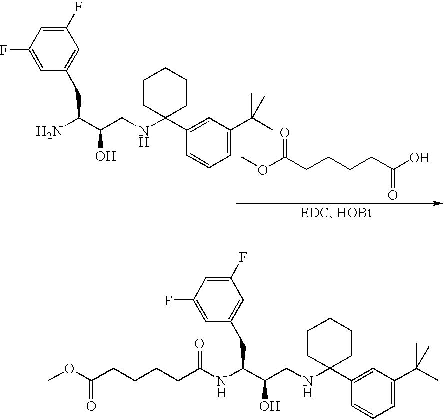

Coupling performed was analogous to that of 4-[3-[1-(3-tert-Butyl-phenyl)-cyclohexylamino]-1-S-(3,5-difluoro-benzyl)-2-R-hydroxy-propylcarbamoyl]-butyric acid methyl ester. Purification performed by column chromatography using 10% MeOH/methylene chloride as the mobile phase yielding 5-[3-[1-(3-tert-Butyl-phenyl)-cyclohexylamino]-1-S-(3,5-difluoro-benzyl)-2-R-hydroxy-propylcarbamoyl]-pentanoic acid methyl ester: retention time=2.15 min, method [1]. ¹H NMR (300 MHz, CDCl₃); δ 7.50 (s, 1H), 7.33-7.23 (m, 3H), 6.71-6.59 (m, 3H), 6.25 (d, J=8.7 Hz, 1H), 4.17-4.12 (m, 1H), 3.74 (s, 3H), 3.43-3.40 (m, 2H), 2.85 (dd, J=15.5 Hz, 1H), 2.69 (dd, J=14.9 Hz, 2H), 2.43-2.22 (m, 6H), 2.13-1.85 (m, 8H), 1.72-1.35 (m, 14H), 1.34 (s, 9H); ¹³C NMR (75 MHz, CDCl₃); δ 173.8, 172.6, 164.4 (d, J=12.8 Hz), 161.1 (d, J=12.8 Hz), 151.1, 142.1 (t, J=8.8 Hz), 127.9, 123.7, 123.6, 123.5, 112.1, 112.0, 111.9, 111.8, 101.8 (t, J=24.8 Hz), 70.6, 58.2, 52.7, 51.4, 43.5, 36.1, 35.8, 35.6, 35.3, 34.7, 33.5, 33.4, 31.2, 25.6, 24.9, 24.7, 24.2, 24.1, 22.1, 22.0.

### EXAMPLE 291

### PREPARATION OF 5-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-PENTANOIC ACID

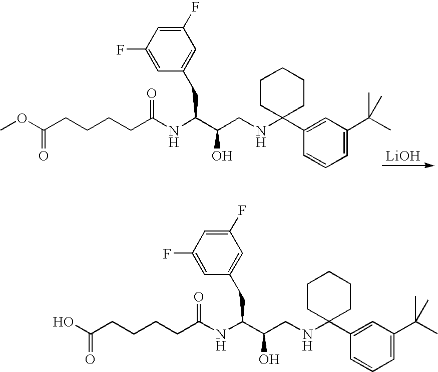

Hydrolysis performed was analogous to that on (S, R)-4-[3-[1-(3-tert-Butyl-phenyl)-cyclohexylamino]-1-(3,5-difluoro-benzyl)-2-hydroxy-propylcarbamoyl]-butyric acid methyl ester. ¹H NMR (300 MHz, CD₃OD); δ 7.69 (s, 1H), 7.54-7.45 (m, 3H), 6.86-6.72 (m, 3H), 3.94-3.91 (m, 1H), 3.66-3.62 (pent, J=1.7 Hz 1H), 3.22 (dd, J=15.3 Hz, 1H), 2.77-2.65 (m, 4H), 2.59 (dd, J=12.3 Hz, 1H), 2.22 (t, J=6 Hz, 2H), 2.06 (t, J=6 Hz, 2H), 1.85 (m, 2H), 1.70-1.38 (m, 18H); ¹³C NMR (75 MHz, CD₃OD); δ 175.4, 175.3, 164.4 (d, J=12.8 Hz), 161.1 (d, J=12.8 Hz), 152.4, 142.8 (t, J=8.8 Hz), 133.8, 128.8, 126.0, 124.8, 124.5, 111.7, 111.3, 101.2 (t, J=24.8 Hz), 69.1, 64.1, 60.0, 53.0, 44.6, 35.5, 35.0, 34.4, 33.7, 33.0, 32.0, 30.3, 24.8, 24.7, 23.9, 21.8, 19.4, 19.3.

### EXAMPLE 292

### PREPARATION OF HEXANEDIOIC-[3-[1-(3-TERT-BUTYL-PHENYL)-CYCLOHEXYLAMINO]-1-S(3,5-DIFLUORO-BENZYL)-2-R-HYDROXY-PROPYL]-AMIDE

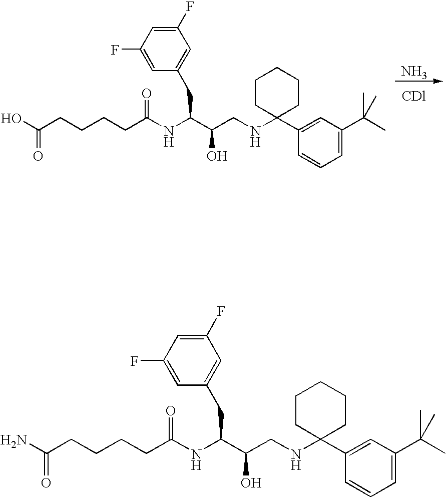

Amination performed was analogous to that on 4-[3-[1-(3-tert-Butyl-phenyl)-cyclohexylamino]-1-S-(3,5-difluoro-benzyl)-2-R-hydroxy-propylcarbamoyl]-butyric acid. ¹H NMR (300 MHz, CDCl₃); δ 7.57 (s, 1H), 7.37-7.25 (m, 3H), 6.68-6.50 (m, 3H), 3.98 (m, 1H), 3.65 (m, 1H), 2.88 (dd, J=12.5, 3 Hz, 1H), 2.61 (dd, J=12.5, 3 Hz, 1H), 2.55-2.47 (m, 1H), 2.46-1.88 (m, 10H), 1.56-1.24 (m, 16H); ¹³C NMR (75 MHz, CDCl₃); δ 173.8, 173.7, 164.3 (d, J=12.8 Hz), 161.0 (d, J=12.8 Hz), 151.8, 142.5 (t, J=8.8 Hz), 137.3, 128.5, 125.1, 124.3, 121.4, 112.0, 111.7, 101.9, 101.5, 69.5, 61.6, 60.3, 52.9, 52.8, 49.2, 48.9, 44.7, 36.4, 35.7, 34.7, 34.0, 31.2, 25.2, 25.0, 24.9.

### EXAMPLE 293

### PREPARATION OF 6-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-HEXANOIC ACID METHYL ESTER

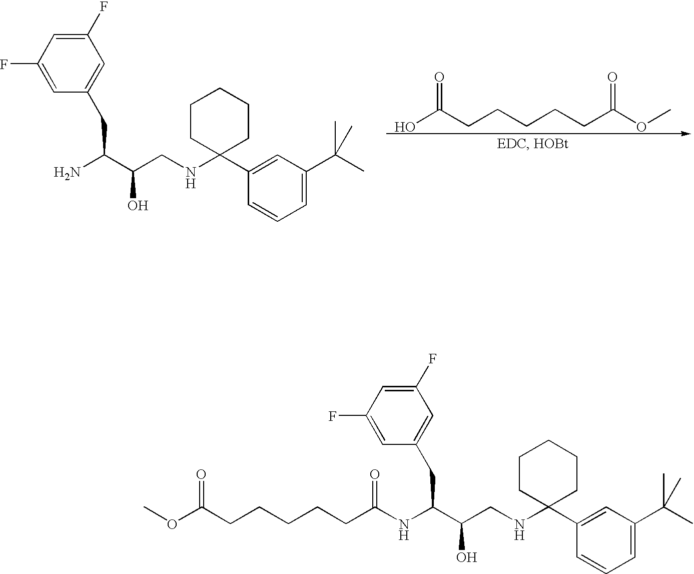

Coupling performed was analogous to that of 4-[3-[1-(3-tert-Butyl-phenyl)-cyclohexylamino]-1-S-(3,5-difluoro-benzyl)-2- R-hydroxy-propylcarbamoyl]-butyric acid methyl ester. Purification performed by column chromatography using 10% MeOH/methylene chloride as the mobile phase yielding 5-[3-[1-(3-tert-Butyl-phenyl)-cyclohexylamino]-1-S-(3,5-difluoro-benzyl)-2-R-hydroxy-propylcarbamoyl]-hexanoic acid methyl ester: retention time=2.18 min, method [1], [M+H]⁺=587.3.

### EXAMPLE 294

### PREPARATION OF 3-ACETYLAMINO-N-[3-[1-(3-TERT-BUTYL-PHENYL)-CYCLOHEXYLAMINO]-1-S(3,5-DIFLUORO-BENZYL)-2-R-HYDROXY-PROPYL]-PROPIONAMIDE

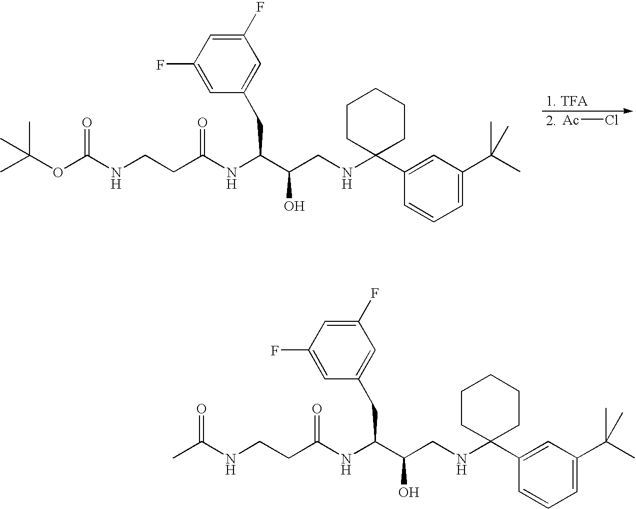

{2-[3-[1-(3-tert-Butyl-phenyl)-cyclohexylamino]-1-S-(3,5-difluoro-benzyl)-2-S-hydroxy-propylcarbamoyl]-ethyl}-carbamic acid tert-butyl ester (0.08 mmol, 50 mg) was charged to a flame dried, 10 mL vial and dissolved in methylene chloride (2 mL). The vial was chilled to 0° C. before the addition of trifluoroacetic acid (3 mL). The cooling bath was removed and the solution was stirred for 3 h before being concentrated under reduced pressure to remove both solvent and TFA. The remaining crust was placed on high vacuum to ensure complete removal of TFA before being dissolved in 1 mL of pyridine. A drop of acetyl chloride was added and the solution was allowed to stir for 16 h. The reaction mixture was diluted with ethyl acetate and rinsed with aqueous, saturated ammonium chloride and saturated copper sulfate before being dried over sodium sulfate. The organic was concentrated under reduced pressure and the residue was purified using preparative TLC with a mobile phase of 7% methanol in methylene chloride: retention time=2.052 min, method [1]; [M+H]⁺ of 544.2.

### EXAMPLE 295

### PREPARATION OF 5-ACETYLAMINO-PENTANOICACID-[3-[1-(3-TERT-BUTYL-PHENYL)-CYCLOHEXYLAMINO]-1-S(3,5-DIFLUORO-BENZYL)-2-R-HYDROXY-PROPYL]-AMIDE

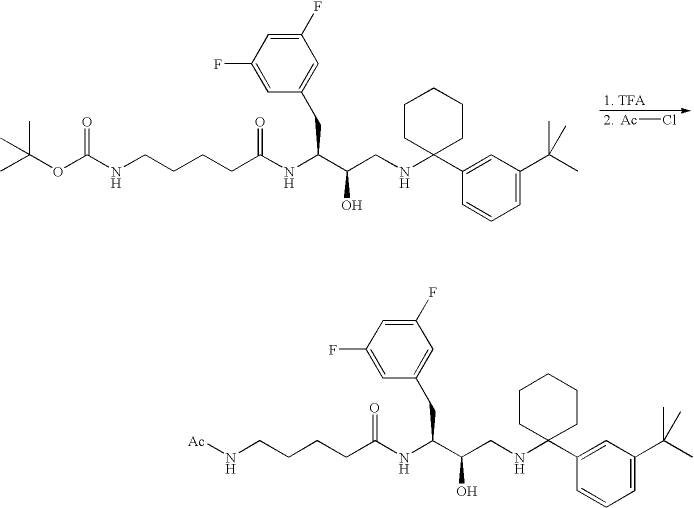

Boc was removed and amine acylated as described for 3-Acetylamino-N-[3-[1-(3-tert-butyl-phenyl)-cyclohexylamino]-1-S-(3,5-difluoro-benzyl)-2-R-hydroxy-propyl]-propionamide: [M+H]⁺ 572.3; retention time=1.883 min, method [1].

### EXAMPLE 296

### PREPARATION OF 4-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-BUTYRIC ACID ISOPROPYL ESTER

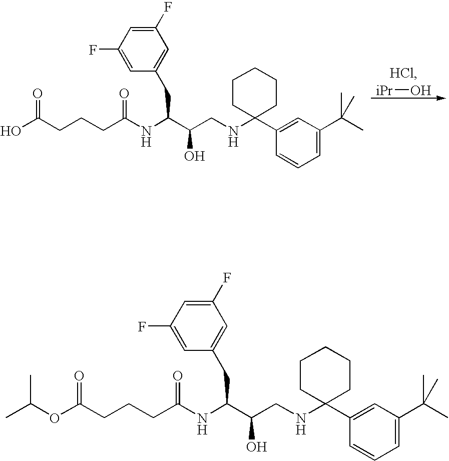

An oven dried, 20 mL vial was charged with 4-[3-[1-(3-tert-Butyl-phenyl)-cyclohexylamino]-1-S-(3,5-difluoro-benzyl)-2-R-hydroxy-propylcarbamoyl]-butyric acid (0.1 mmol, 55 mg) as a white powder. Isopropyl alcohol (10 mL) was added, and the suspension was chilled to 0° C. before HCl was bubbled into the mixture as a gas. After 10 min of HCl application, the solution was nearly homogenous and the gas bubbling was ended. The reaction mix was warmed to 23° C. and stirred for 16 h. The reaction mix was concentrated under reduced pressure and the residue was partitioned between am aqueous, saturated solution of sodium bicarbonate and ethyl acetate. The organic solution was dried over sodium sulfate and concentrated under reduced pressure yielding a white solid as pure product. [M+H]⁺ 587.2; retention time=2.286 min, method [1].

### EXAMPLE 297

### PREPARATION OF 4-[1-(3,5-DIFLUORO-BENZYL)-3-(7-ETHYL-1,2,3,4-TETRAHYDRO-NAPHTHALEN-1-SYLAMINO)-2-R-HYDROXY-PROPYLCARBAMOYL]-BUTYRIC ACID

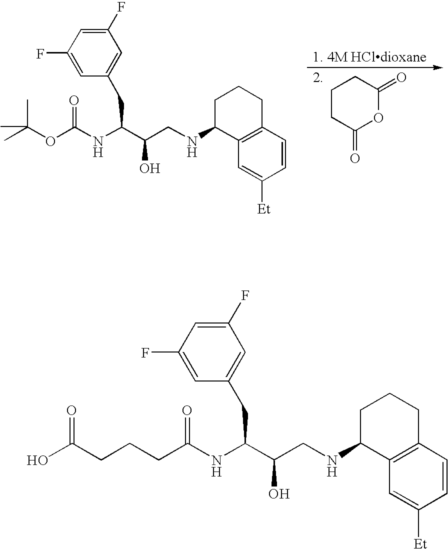

A flame dried 25 mL RBF was charged with [1-(3,5-Difluoro-benzyl)-3-(7-ethyl-1,2,3,4-tetrahydro-naphthalen-1-S-ylamino)-2-R-hydroxy-propyl]-carbamic acid tert-butyl ester (0.42 mmol, 0.2 g) and methylene chloride was added (5 mL). The solution was chilled to 0° C. and 4M HCl in dioxane (4 mL) was added. The cooling bath was removed and the solution was stirred for 1 h at 23° C. The reaction mix was concentrated under reduced pressure and the residue was dissolved in dichloroethane (3 mL). Hunig's base (1.2 mmol, 0.21 mL) was added, followed by 1.2 equiv. of the anhydride. The reaction mixture was allowed to stir for 16 h at 23° C. The mixture was taken up in 10% aqueous solution of potassium carbonate and rinsed with ethyl ether before the aqueous was brought to pH=2 and extracted with ethyl acetate. The organic extract was dried over sodium sulfate and concentrated under reduced pressure yielding pure acid product: [M+H]⁺ 587.2; retention time=2.286 min, method [1].

### EXAMPLE 298

### PREPARATION OF N-[3-[1-(3-TERT-BUTYL-PHENYL)-CYCLOHEXYLAMINO]-1-S(3,5-DIFLUORO-BENZYL)-2-R-HYDROXY-PROPYL]-4-SULFAMOYL-BUTYRAMIDE

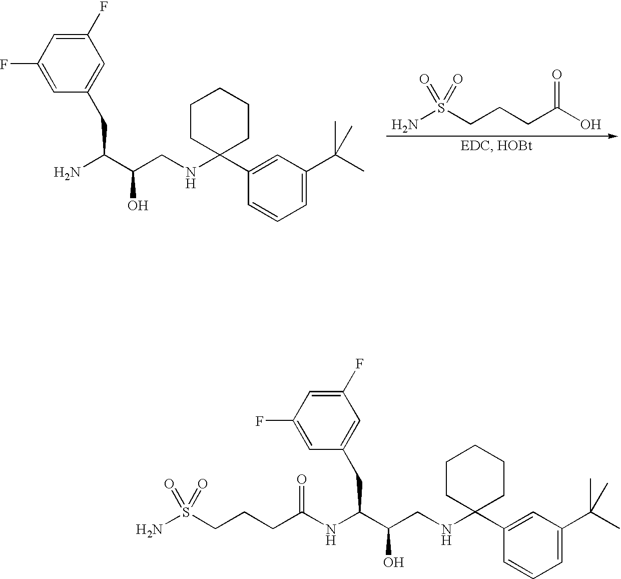

3-Amino-1-[1-(3-tert-butyl-phenyl)-cyclohexylamino]-4-S-(3,5-difluoro-phenyl)-R-butan-2-ol (0.47 mmol, 0.20 g) was charged to a 50 mL, flame dried round bottom flask and taken up in CH₂Cl₂ (10 mL). 4-Sulfamoyl-butyric acid (0.47 mmol, 0.08 g) was added, followed by N-methylmorpholine (1.88 mmol, 190 mg, 0.21 mL), HOBt (0.56 mmol, 76 mg), and EDC (0.56 mmol, 108 mg). The homogenous reaction was stirred at 23° C. for 16h under nitrogen before being quenched by the addition of an aqueous, saturated solution of ammonium chloride. The mixture was extracted with ethyl acetate (2×50 mL) and the organic solution was rinsed with aqueous, saturated solutions of sodium bicarbonate and sodium chloride (50 mL each) in sequence. The ethyl acetate solution was dried over sodium sulfate and concentrated under reduced pressure to yield a brown oil as the crude product. The crude product was purified by column chromatography using a mobile phase of 7% MeOH/methylene chloride yielding N-[3-[1-(3-tert-Butyl-phenyl)-cyclohexylamino]-1-S-(3,5-difluoro-benzyl)-2-R-hydroxy-propyl]-4-sulfamoyl-butyramide as white foam: [M+H]⁺ 580.2; retention time=1.866 min, method [1].

### EXAMPLE 299

### PREPARATION OF {[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-METHOXY}-ACETIC ACID

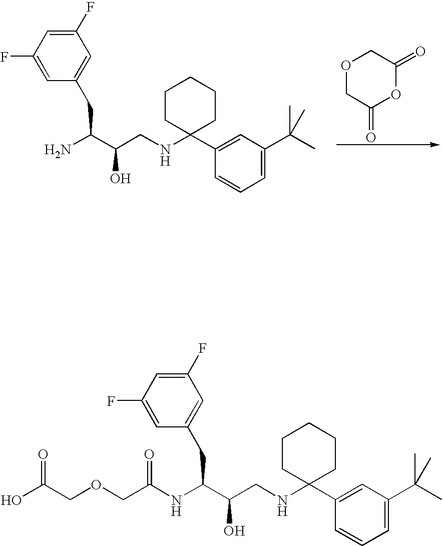

The title compound was prepared in manner analogous to the synthesis of (S,R)-4-[1-(3,5-Difluoro-benzyl)-3-(7-ethyl-1,2,3,4-tetrahydro-naphthalen-1-ylamino)-2-hydroxy-propylcarbamoyl]-butyric acid. Purification of the crude acid was done via HPLC using a C18 column and a mobile phase of 25 to 45% MeCN at 18 mL/min: [M+H]⁺=547.2; retention time=1.891 min, method [1].

### EXAMPLE 300

### PREPARATION OF (2-[3-[1-(3-TERT-BUTYL-PHENYL)-CYCLOHEXYLAMINO]-1-S-(3,5-DIFLUORO-BENZYL)-2-R-HYDROXY-PROPYLCARBAMOYL]-PYRROLIDIN-1-YL}-ACETIC ACID METHYL ESTER

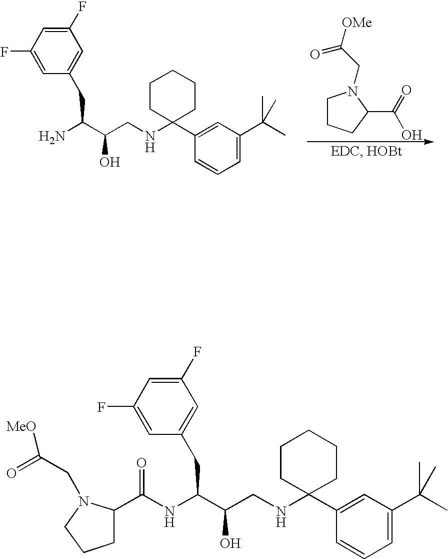

Coupling performed was analogous to that of (S, R)-4-[3-[1-(3-tert-Butyl-phenyl)-cyclohexylamino]-1-(3,5-difluoro-benzyl)-2-hydroxy-propylcarbamoyl]-butyric acid methyl ester. Purification performed by column chromatography using 4% MeOH/methylene chloride as the mobile phase yielding {2-[3-[1-(3-tert-Butyl-phenyl)-cyclohexylamino]-1-S-(3,5-difluoro-benzyl)-2-R-hydroxy-propylcarbamoyl]-pyrrolidin-1-yl}-acetic acid methyl ester: retention time=1.712 min, method [1]; [M+H]⁺=600.3.

### EXAMPLE 301

### PREPARATION OF {2-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-PYRROLIDIN-1-YL}-ACETIC ACID

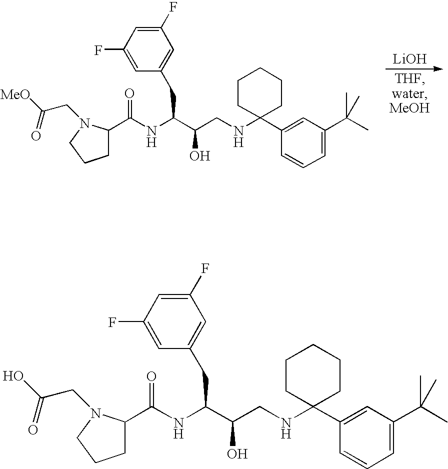

Ester hydrolysis performed in an analogous method to that used in the preparation of (S,R)-4-[3-[1-(3-tert-Butyl-phenyl)-cyclohexylamino]-1-(3,5-difluoro-benzyl)-2-hydroxy-propylcarbamoyl]-butyric acid. Product was extracted efficiently into the ethyl ether layer of the workup extraction so the organic was concentrated under reduced volume. The resulting white solid was pure {2-[3-[1-(3-tert-Butyl-phenyl)-cyclohexylamino]-1-S-(3,5-difluoro-benzyl)-2-R-hydroxy-propylcarbamoyl]-pyrrolidin-1-yl}-acetic acid. Pyrrolidine diastereomer A: retention time=1.636, method [1]; [M+H]⁺=586.3. Pyrrolidine diastereomer B: retention time=1.736, method [1]; [M+H]⁺=586.3.

### EXAMPLE 302

### PREPARATION OF 1-CARBAMOYLMETHYL-PYRROLIDINE-2-CARBOXYLIC ACID [3-[1-(3-TERT-BUTYL-PHENYL)-CYCLOHEXYLAMINO]-1-S(3,5-DIFLUORO-BENZYL)-2-R-HYDROXY-PROPYL]-AMIDE

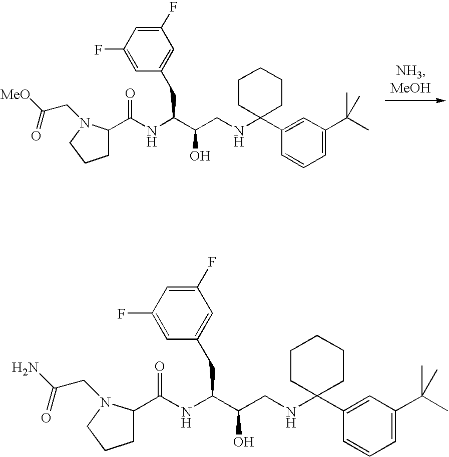

A 20 mL flame dried vial was charged with {2-[3-[1-(3-tert-Butyl-phenyl)-cyclohexylamino]-1-S-(3,5-difluoro-benzyl)-2-R-hydroxy-propylcarbamoyl]-pyrrolidin-1-yl}-acetic acid methyl ester (0.17 mmol, 100 mg) as a sticky white foam. NH₃.MeOH (5 mL of a 7M solution) was added and the vial was sealed under a Teflon topped cap and heated to 110° C. The heating was maintained for 16 h until it was cooled to 23° C. and concentrated under reduced pressure. The resulting residue was pure product. Pyrrolidine diastereomer A: retention time=1.595 min, method [1]; [M+H]⁺=585.3. Pyrrolidine diastereomer B: retention time=1.664 min, method [1]; [M+H]⁺=586.3.

### EXAMPLE 303

### PREPARATION OF 4-TERT-BUTOXYCARBONYLAMINO-4-[3-[1-(3-TERT-BUTYL-PHENYL)-CYCLOHEXYLAMINO]-1-S(3,5-DIFLUORO-BENZYL)-2-R-HYDROXY-PROPYLCARBAMOYL]-BUTYRIC ACID TERT-BUTYL ESTER

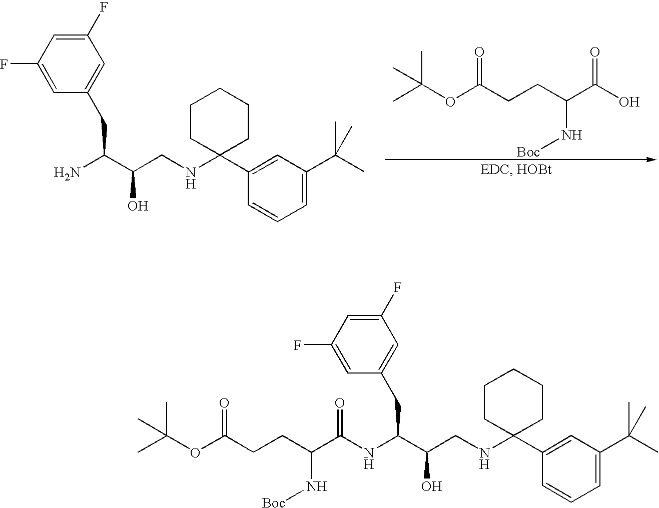

Coupling performed was analogous to that used in the synthesis of 4-[3-[1-(3-tert-Butyl-phenyl)-cyclohexylamino]-1-S-(3,5-difluoro-benzyl)-2-R-hydroxy-propylcarbamoyl]-butyric acid methyl ester. Purification performed by column chromatography using 40 to 60% ethyl acetate in hexanes as the mobile phase yielding 4-tert-Butoxycarbonylamino-4-[3-[1-(3-tert-butyl-phenyl)-cyclohexylamino]-1-S-(3,5-difluoro-benzyl)-2-R-hydroxy-propylcarbamoyl]-butyric acid tert-butyl ester. Retention time=2.514 min, method [1]; [M+H]⁺=716.4.

### EXAMPLE 304

### PREPARATION OF 4-[3-[1-(3-TERT-BUTYL-PHENYL)-CYCLOHEXYLAMINO]-1-(3,5-DIFLUORO-BENZYL)-2-HYDROXY-PROPYLCARBAMOYL]-2,2-DIMETHYL-BUTYRIC ACID

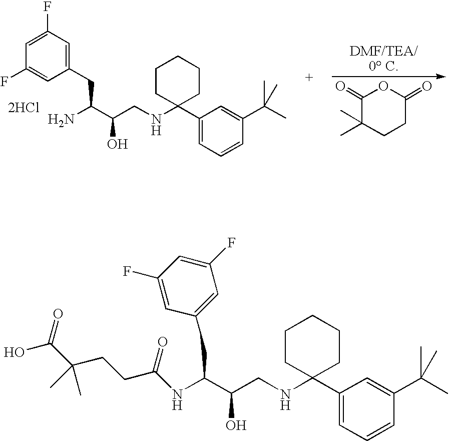

A solution of 3,3-dimethyl-dihydro-pyran-2,6-dione (1.5 mmol) in DMF (6 mL) was added to a solution of 3-amino-1-[1-(3-tert-butyl-phenyl)-cyclohexylamino]-4-(3,5-difluoro-phenyl)-butan-2-ol dihydrochloride salt (1 mmol) and triethylamine (0.15 mmol) in DMF (2 mL) at 0° C. The reaction was allowed to come to room temperature and stirred under nitrogen gas overnight. The reaction was treated with H₂O (50 mL) and 4:1 CHCl₃:IPA (50 mL), the aqueous layer discarded, dried with sodium sulfate, and concentrated. The obtained residue was purified by reverse-phase HPLC. Retention time (min)=2.048, method [1]; ¹H NMR (300 MHz, CD₃OD); δ 7.65 (s, 1H), 7.54-7.38 (m, 3H), 6.82-6.72 (m, 3H), 3.86 (m, 1H), 3.54 (m, 1H), 3.23 (dd, 1H, J=13.6, 3.4 Hz), 2.76-2.47 (m, 5H), 2.01 (t, 2H, J=8.5 Hz), 1.97-1.75 (m, 3H) 1.68-1.38 (m, 8H), 1.36 (s, 9H), 1.09, (d, 6H, J=5.4 Hz); MS (ESI) 573.3.

### EXAMPLE 305

### PREPARATION OF 4-[3-[1-(3-TERT-BUTYL-PHENYL)-CYCLOHEXYLAMINO]-1-(3,5-DIFLUORO-BENZYL)-2-HYDROXY-PROPYLCARBAMOYL]-ADAMANTANE-1-CARBOXYLIC ACID METHYL ESTER.

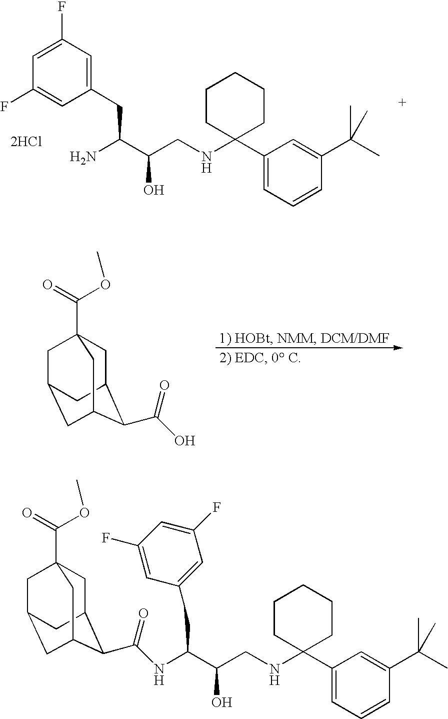

Adamantane-1,4-dicarboxylic acid 1-methyl ester (1 mmol) was dissolved in dichloromethane (10 mL) and stirred under nitrogen at 0° C. To this mixture was added 3-amino-1-[1-(3-tert-butyl-phenyl)-cyclohexylamino]-4-(3,5-difluoro-phenyl)-butan-2-ol dihydrochloride salt (1 mmol), HOBt (2 mmol), and N-methylmorpholine (5 mmol). DMF (2 mL) was added to solubilize reaction contents. This mixture was stirred at 0° C. for 30 min to 1 h followed by addition of EDC (2 mmol). The reaction was stirred overnight and allowed to come to room temperature. The contents were stripped down and taken up in equal portions of water and CHCl₃/IPA (4:1). The aqueous layer was discarded and the organic layer was washed with sat. NaHCO₃, (25 mL), H₂O (25 mL), brine (25 mL), and dried with sodium sulfate. The organics were removed via rotary evaporation. The obtained residue was purified by reverse-phase HPLC. Retention time (min)=2.366, method [1]; ¹H NMR (300 MHz, CD₃OD); δ 7.66 (s, 1H), 7.53-7.31 (m, 3H), 6.82-6.72 (m, 3H), 3.90 (m, 1H), 3.66 (s, 3H), 3.63 (m, 1H), 3.20 (m, 1H), 2.75-2.50 (m, 5H), 2.11-1.19 (m, 22H), 1.36 (d, 9H, J=2.3 Hz); MS (ESI) 651.3.

### EXAMPLE 306

### PREPARATION OF 4-[3-[1-(3-TERT-BUTYL-PHENYL)-CYCLOHEXYLAMINO]-1-(3,5-DIFLUORO-BENZYL)-2-HYDROXY-PROPYLCARBAMOYL]-4-METHYL-PENTANOIC ACID METHYL ESTER

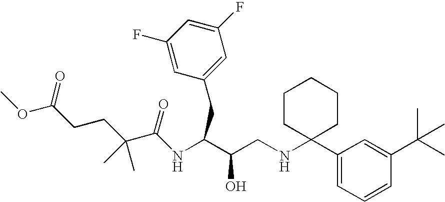

Compound was prepared in an identical manner to Example 305 using 2,2-dimethyl-pentanedioic acid 5-methyl ester as the coupling species. Retention time (min)=2.242, method [1]; MS (ESI) 587.3.

### EXAMPLE 307

### PREPARATION OF {4-[3-[1-(3-TERT-BUTYL-PHENYL)-CYCLOHEXYLAMINO]-1-(3,5-DIFLUORO-BENZYL)-2-HYDROXY-PROPYLAMINO]-PHENYL}-ACETIC ACID.

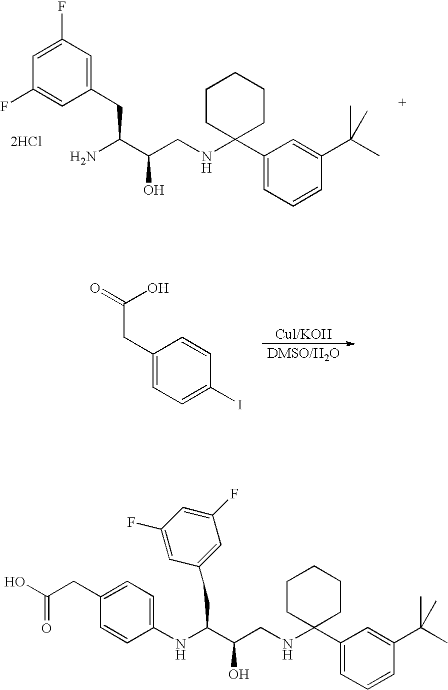

3-Amino-1-[1-(3-tert-butyl-phenyl)-cyclohexylamino]-4-(3,5-difluoro-phenyl)-butan-2-ol dihydrochloride salt (1 mmol), 4-iodophenylacetic acid (1 mmol), and potassium hydroxide (5 mmol) were added to round bottomed flask equipped with stirbar. DMSO (5 mL) and H₂O (5 mL) were added and the mixture dissolved. Copper iodide (10%) was added and the mixture heated for 16 h at 90° C. The reaction was extracted with DCM (2×10 mL), then neutralized with 1M HCl and extracted with 4:1 CHCl₃/IPA. Both organic fractions showed potential product so they were combined, dried with sodium sulfate, and rotovapped to dryness yielding brown oil. This residue was purified by reverse-phase HPLC. Retention time (min)=2.274, method [1]; ¹H NMR (300 MHz, CD₃OD); δ 7.54 (s, 1H), 7.46-7.27 (m, 3H), 6.94 (d, 2H, J=7.8 Hz), 6.76-6.59 (m, 3H), 6.36 (d, 2H, J=7.8 Hz), 3.58-3.43 (m, 2H), 3.41 (s, 2H), 3.03 (d, 1H), J=13.7 Hz), 2.87 (d, 1H, J=13.7 Hz), 2.76-2.45 (m, 4H), 1.95-1.54 (m, 4H), 1.39-1.06 (m, 11H). ¹³C NMR (75 MHz, CD₃OD); δ 174.7, 162.7 (dd, 2C, J=248.2, 13.5 Hz), 158.2, 152.2, 146.0, 142.6 (t, 1C, J=9.7 Hz), 133.1, 129.6, 128.9, 126.0, 124.6, 124.3, 123.0, 112.6, 111.8 (dd, 2C, J=17.1, 7.4 Hz), 100.9 (t, 1C, J=25.7 Hz), 69.7, 64.0, 57.3, 45.1, 39.5, 35.9, 34.3, 32.6, 32.5, 30.0, 24.6, 21.7; MS (ESI) 565.2.

### EXAMPLE 308

### PREPARATION OF 3-{4-[3-[1-(3-TERT-BUTYL-PHENYL)-CYCLOHEXYLAMINO]-1-(3,5-DIFLUORO-BENZYL)-2-HYDROXY-PROPYLAMINO]-PHENYL}-PROPIONIC ACID

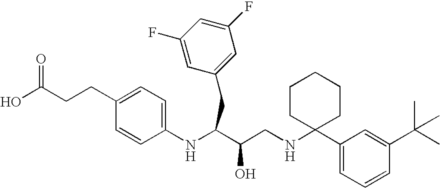

Compound was prepared in an identical manner to Example 307 using 3-(4-iodo-phenyl)-propionic acid as the coupling species. Retention time (min)=2.106, method [1]; ¹H NMR (300 MHz, CD₃OD); δ 7.55 (s, 1H), 7.46-7.27 (m, 3H), 6.90 (d, 2H), J=7.8 Hz), 6.76-6.59 (m, 3H), 6.36 (d, 2H, J=7.8 Hz), 3.58-3.43 (m, 2H), 3.02 (dd, 1H, J=14.0, 4.0 Hz), 2.88 (dd, 1H, J=13.0, 2.7 Hz), 2.76-2.46 (m, 6H), 1.95-1.54 (m, 4H), 1.39-1.06 (m, 11H). ¹³C NMR (75 MHz, CD₃OD); δ 175.4, 162.7 (dd, 2C, J=248.2, 13.5 Hz), 159.4, 152.2, 147.5, 145.0, 142.6 (t, 1C, J=9.7 Hz), 133.3, 129.6, 128.9, 126.0, 124.6, 124.3, 123.0, 112.6, 111.8 (dd, 2C, J=17.1, 7.4 Hz), 100.9 (t, 1C, J=25.7 Hz), 69.7, 64.0, 57.3, 45.1, 39.5, 35.9, 35.5, 34.3, 32.6, 32.5, 30.1, 29.6, 24.6, 21.7; MS (ESI) 579.3.

### EXAMPLE 309

### PREPARATION OF 2-{3-[3-[1-(3-TERT-BUTYL-PHENYL)-CYCLOHEXYLAMINO]-1-(3,5-DIFLUORO-BENZYL)-2-HYDROXY-PROPYLAMINO]-PHENYL}-N,N-DIPROPYL-ACETAMIDE

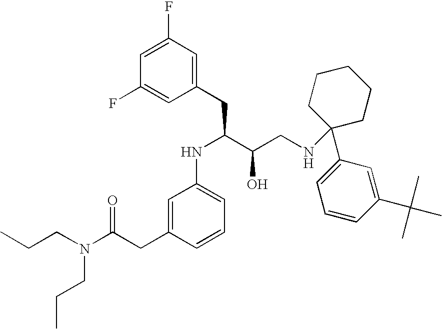

Compound was prepared in an identical manner to Example 307 using 2-(3-iodo-phenyl)-N,N-dipropyl-acetamide as the coupling species. Retention time (min)=2.529, method [1]; ¹H NMR (300 MHz, CD₃OD); δ 7.56 (s, 1H), 7.46-7.27 (m, 3H), 6.96 (t, J=7.6 Hz), 6.76-6.59 (m, 3H), 6.46 (d, 1H, J=7.2 Hz), 6.33 (s, 1H), 6.29 (d, 1H, J=7.9 Hz), 3.57 (s, 2H), 3.55-3.45 (m, 2H), 3.31-3.17 (m, 5H), 3.01 (dd, 1H, J=13.8, 3.8), 2.87 (dd, 1H, J=12.6, 2.1 Hz), 2.79-2.51 (m, 4H), 1.96-1.30 (m, 12H), 1.28 (s, 9H); MS (ESI) 648.3.

### EXAMPLE 310

### PREPARATION OF 4-[3-[1-(3-TERT-BUTYL-PHENYL)-CYCLOHEXYLAMINO]-1 (3,5-DIFLUORO-BENZYL)-2-HYDROXY-PROPYLAMINO]-BENZOIC ACID

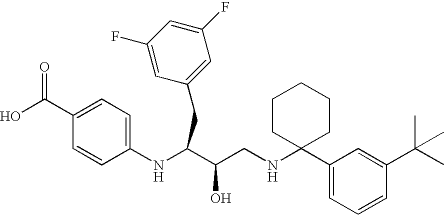

Compound was prepared in an identical manner to Example 307 using 4-iodo-benzoic acid as the coupling species. Retention time (min)=1.966, method [1]; MS (ESI) 551.2.

### EXAMPLE 311

### PREPARATION OF 4-[3-[1-(3-TERT-BUTYL-PHENYL)-CYCLOHEXYLAMINO]-1 (3,5-DIFLUORO-BENZYL)-2-HYDROXY-PROPYLAMINO]-N-METHYL-BENZAMIDE

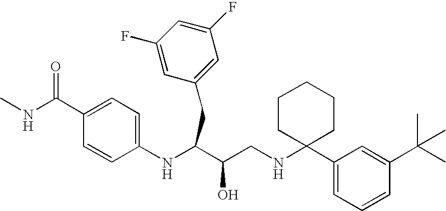

Compound was prepared in an identical manner to Example 307. using 4-iodo-N-methyl-benzamide as the coupling species. Retention time (min)=1.949, method [1]; MS (ESI) 564.3.

### EXAMPLE 312

### PREPARATION OF 4-[3-[1-(3-TERT-BUTYL-PHENYL)-CYCLOHEXYLAMINO]-1 (3,5-DIFLUORO-BENZYL)-2-HYDROXY-PROPYLAMINO]-BENZAMIDE

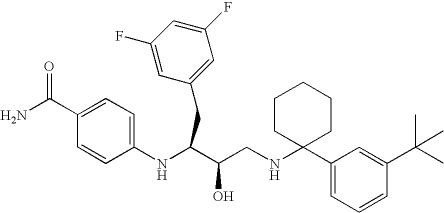

Compound was prepared in an identical manner to Example 307 using 4-iodo-benzamide as the coupling species. Retention time (min)=1.977, method [1]; MS (ESI) 551.2.

### EXAMPLE 313

### PREPARATION OF 4-[3-[1-(3-TERT-BUTYL-PHENYL)-4-HYDROXYIMINO-CYCLOHEXYLAMINO]-1 (3,5-DIFLUORO-BENZYL)-2-HYDROXY-PROPYLCARBAMOYL]-BUTYRIC ACID

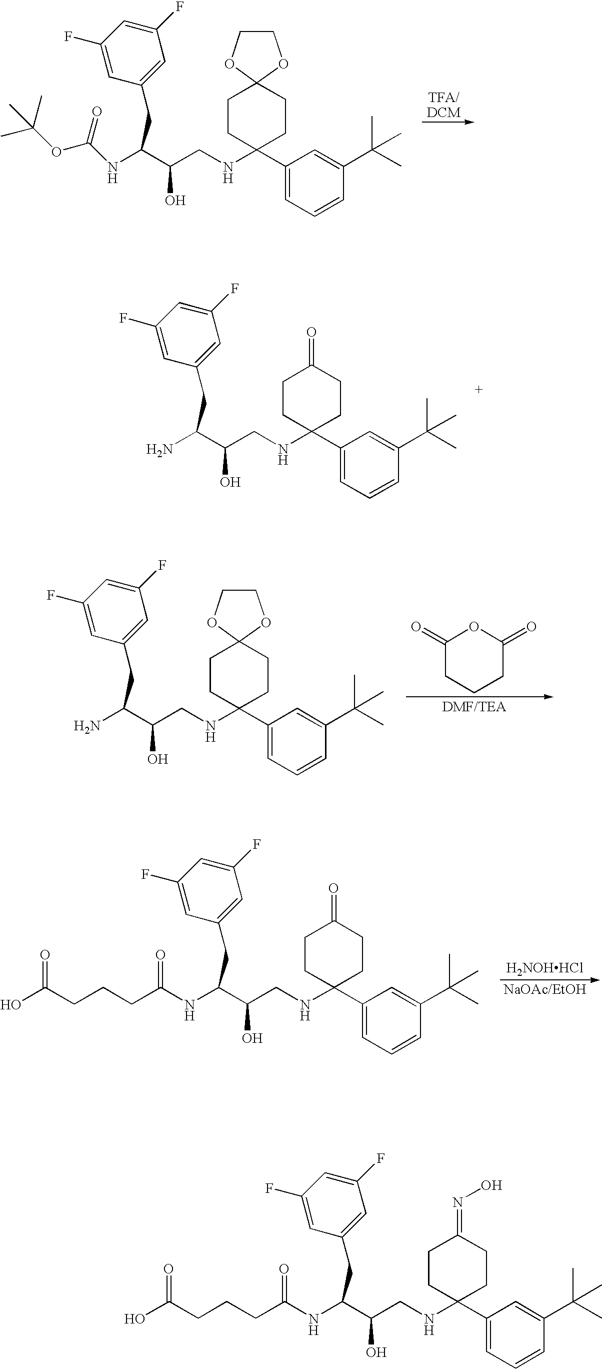

Standard TFA deprotection of [3-[8-(3-tert-Butyl-phenyl)-1,4-dioxa-spiro[4.5]dec-8-ylamino]-1-(3,5-difluoro-benzyl)-2-hydroxy-propyl]-carbamic acid tert-butyl ester yielded mixture of deprotected ketone and deprotected ketal. To a solution of 4-[3-[1-(3-tert-Butyl-phenyl)-4-hydroxyimino-cyclohexylamino]-1 (3,5-difluoro-benzyl)-2-hydroxy-propylcarbamoyl]-butyric acid (1 mmol) (plus ketal by-product) in ethanol (10 mL) was added hydroxylamine hydrochloride (2.5 mmol) and sodium acetate (5 mmol). The reaction mixture was stirred at room temperature for 2.5 h prior to partioning between H₂O and CH₂Cl₂. The organic layer was separated, dried (Na₂SO₄) and concentrated under reduced pressure. The residue was purified by reverse phase HPLC. Retention time (min)=1.594, method [1]; MS (ESI) 574.3.

### EXAMPLE 314

### PREPARATION OF 3-ACETYL-1-BUTYL-1H-INDOLE-6-CARBOXYLIC ACID [3-[1-(3-TERT-BUTYL-PHENYL)-CYCLOHEXYLAMINO]-1-(3,5-DIFLUORO-BENZYL)-2-HYDROXY-PROPYL]-AMIDE

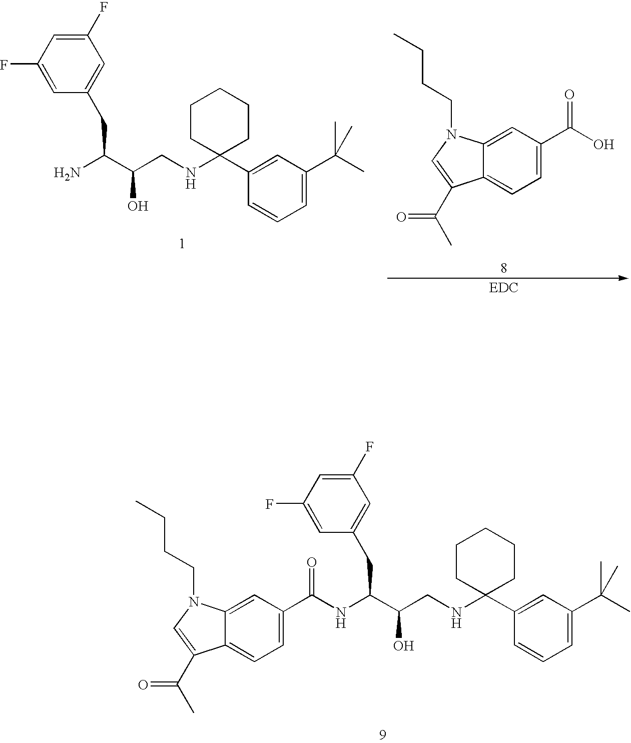

Indole 9 was prepared by standard EDC coupling between amine 1 (described previously) and 3-acetyl-1-butyl-1H-indole-6-carboxylic acid 8. Purified by HPLC. Retention time (min)=2.41, method [1]; ¹H NMR (300 MHz, DMSO-d₆); δ 8.53 (s, 1H), 8.38 (d, J=9 Hz, 1H), 8.17 (d, J=8.4 Hz, 1H), 7.94 (s, 1H), 7.59 (s, 1H), 7.52 (d, J=8.4, 1H), 7.39 (bs, 1H), 7.31 (d, J=4.2, 2H), 6.98 (m, 3H), 5.83 (m, 1H), 4.28 (m, 2H), 4.05 (m, 1H), 3.77 (m, 1H), 3.37 (s, 8H), 3.05 (m, 1H), 2.85 (m, 1H), 2.46 (s, 3H), 1.95 (m, 2H), 1.82 (t, J=7.8, 2H), 1.76 (m, 2H), 1.51 (m, 1H), 1.30 (m, 4H), 1.17 (s, 9H), 1.11-1.07 (m, 2H), 0.92 (t, J=7.2, 3H); MS (ESI) 672.6.

### EXAMPLE 315

### PREPARATION OF N-[3-[1-(3-TERT-BUTYL-PHENYL)-CYCLOHEXYLAMINO]-1 (3,5-DIFLUORO-BENZYL)-2-HYDROXY-PROPYL]-3-METHANESULFONYLAMINO-BENZAMIDE (6)

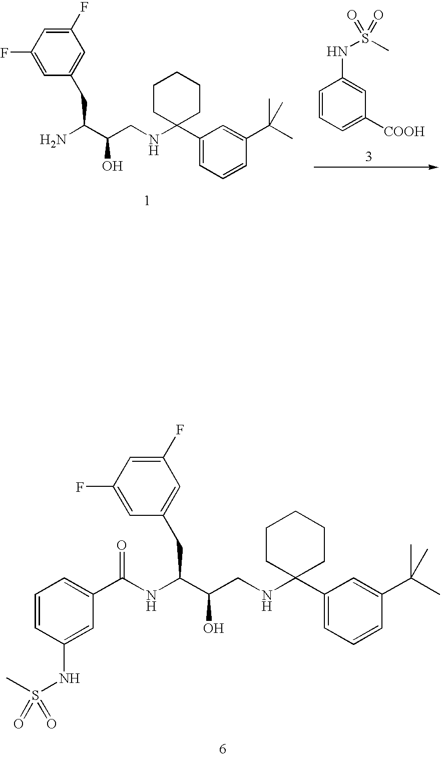

3-Amino-1-[1-(3-tert-butyl-phenyl)-cyclohexylamino]-4-(3,5-difluoro-phenyl)-butan-2-ol (0.125 g, 0.29 mM), acid 3 (0.065 g, 0.30 mM), and NMM (0.2 mL) in CH₂Cl₂ (5 mL) were treated with HOBt (0.047 g, 0.35 mM) and EDC (0.067 g, 0.35 mM) at 0° C. The reaction mixture was stirred over night, at room temperature. Next solvent was stripped and the reaction mixture was partitioned between NaHCO₃ and EtOAc. Organic layer was washed with brine, dried and concentrated; purified by HPLC. Yield 0.110 g (61%). Retention time (min)=2.12, method [1]; ¹H NMR (300 MHz, CDCl₃); δ 8.91 (bs, 1H), 8.45 (s, 1H), 8.02 (bs, 1H), 7.58 (d, J=6.9 Hz, 2H), 7.52-7.49 (m, 1H), 7.44-7.32 (m, 5H), 6.71 (d, J=6 Hz, 2H), 6.58 (dt, J=9, 2 Hz, 1H), 4.28 (bs, 1H), 3.99 (bs, 1H), 2.98 (s, 4H), 2.85-2.76 (m, 2H), 2.58-2.42 (m, 11H), 2.07-2.03 (m, 2H), 1.80-1.74 (m, 2H), 1.61-1.58 (m, 1H), 1.42-1.38 (m, 3H), 1.30 (s, 9H); MS (ESI) 628.3 Acids 3 and 5 were synthesized according to the procedure described in WO 2000055153.

### EXAMPLE 316

### PREPARATION OF N-[3-[1-(3-TERT-BUTYL-PHENYL)-CYCLOHEXYLAMINO]-1-(3,5-DIFLUORO-BENZYL)-2-HYDROXY-PROPYL]-3-(METHANESULFONYL-METHYL-AMINO)-BENZAMIDE

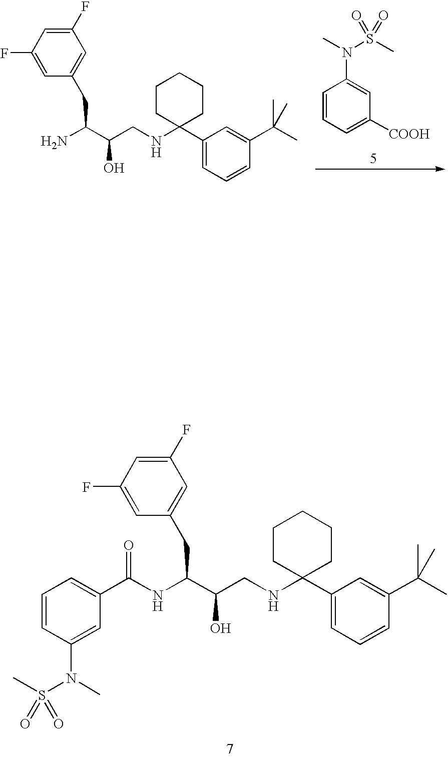

Compound 7 was prepared according to the procedure of EXAMPLE 315. Retention time (min)=2.19, method [1]; ¹H NMR (300 MHz, CDCl₃); δ 9.20 (bs, 1H), 8.12 (bs, 1H), 7.70 (s, 1H), 7.59-7.54 (m, 2H), 7.46-7.32 (m, 2H), 7.35 (m, 3H), 6.74 (d, J.=0.6 Hz, 2H), 6.64 (dt, J.=0.9, 2 Hz, 1H), 4.24 (bs, 1H), 3.89 (bs, 1H), 3.34 (s, 3H), 3.09-3.04 (m, 1H), 2.86 (s, 4H), 2.77-2.74 (m, 1H), 2.59-2.47 (m, 12H), 2.09-2.04 (m, 2H), 1.81-1.77 (m, 2H), 1.60 (bs, 1H), 1.50-1.40 (m, 3H), 1.30 (s, 9H); MS (ESI) 642.3

### EXAMPLE 317

### PREPARATION OF [3-[1-(3-TERT-BUTYL-PHENYL)-CYCLOHEXYLAMINO]-1-(3,5-DIFLUORO-BENZYL)-2-HYDROXY-PROPYL]-UREA

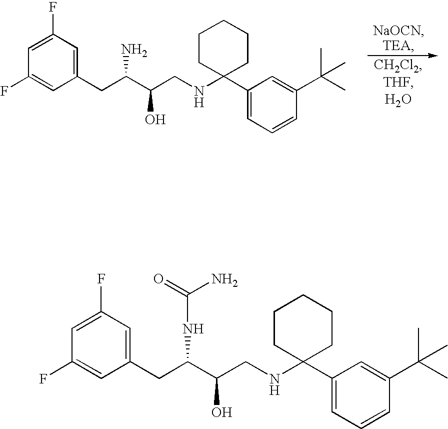

Sodium cyanate (32 mg, 492 umol) was added to a solution of 3-amino-1-[1-(3-tert-butyl-phenyl)-cyclohexylamino]-4-(3,5-difluoro-phenyl)-butan-2-ol dihydrochloride salt (200 mg, 397 umol) and triethylamine (0.08 mL, 574 umol) in methylene chloride (2 mL) and water (2 mL). Three additional portions of sodium cyanate (200 mg, 3.08 mmol) were added after each subsequent 24 h period. After stirring for 4 d, the solution was concentrated and the residue was flash chromotographed with 99:1:0.1, 49:1:0.1, 24:1:0.1, 23:2:0.2, 22:3:0.3, 21:4:0.4, 4:1:0.1, 7:3:0.3, and 3:2:0.2 methylene chloride:methanol:concentrated ammonium hydroxide as the eluant to yield [3-[1 (3-tert-butyl-phenyl)-cyclohexylamino]-1-(3,5-difluoro-benzyl)-2-hydroxy-propyl]-urea. Method [1] Retention time 1.87 min by HPLC and 1.92 min by MS (M+=474).

### EXAMPLE 318

### PREPARATION OF 1-BUT-3-ENYL-3,5-DIFLUOROBENZENE

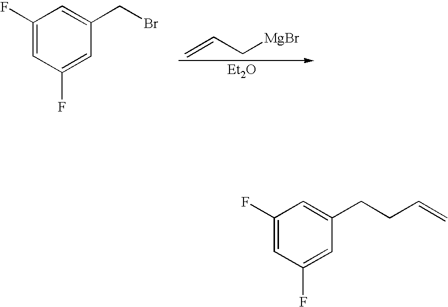

1-Bromomethyl-3,5-difluorobenzene (10.75 g, 51.9 mmol) was added dropwise slowly to a stirring solution of allylmagnesium bromide (Aldrich, 1.0 M solution in diethyl ether, 78 mL, 78 mmol) at room temperature. Upon complete addition, the reaction mixture was stirred at room temperature for 2.5 h. Reaction was quenched by slow addition of 1 N HCl (40 mL). Diethyl ether (30 mL) was added, and the organics were separated, washed (brine), dried (magnesium sulfate), filtered and concentrated. Fractional distillation (55-60° C. at 13 torr) afforded product as a clear, colorless liquid (5.3 g, 60%): R_{f}=0.77 (hexanes).

### EXAMPLE 319

### PREPARATION OF 2-[2-(3,5-DIFLUOROPHENYL)ETHYL]OXIRANE

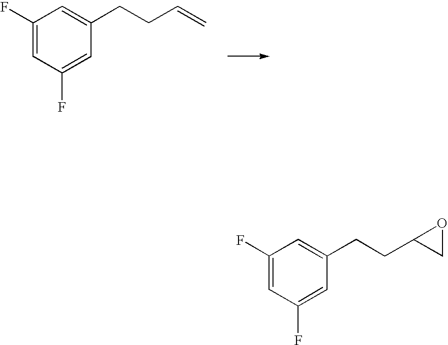

m-Chloroperbenzoic acid (22 g, Lancaster, 50-55wt %, 64 mmol) was dissolved in dichloromethane (150 mL), and cooled to 0° C. 1 But-3-enyl-3,5-difluorobenzene (5.3 g, 31.5 mmol) in dichloromethane (10 mL) was added, and the mixture was allowed to warm to rt overnight. The reaction was quenched with saturated Na₂SO₃ (70 mL) and saturated NaHCO₃ (70 mL), and the resulting mixture was stirred for 2 h. The organics were separated, washed with saturated NaHCO₃ (40 mL), brine (50 mL), dried (magnesium sulfate), filtered and concentrated. The residue was dissolved in minimal cold hexanes and filtered. The filtrate was concentrated yielding desired product (4.0 g, 70%): retention time (min)=1.977; ¹³C NMR (75 MHz, CDCl₃); δ 162.9 (dd, J=246.4, 12.9 Hz, 2C), 145.0 (t, J=8.9 Hz, 1C), 111.0 (dd, J=16.7, 7.4 Hz, 2C), 101.4 (t, J=25.1 Hz, 1C), 51.2, 47.0, 33.5, 31.9; MS (ESI) 167.

### EXAMPLE 320

### PREPARATION OF 4-(3,5-DIFLUORO-PHENYL)-1-{1-[3-(2,2-DIMETHYL-PROPYL)-PHENYL]-CYCLOHEXYLAMINO}-3-(2,2,2-TRIFLUORO-ETHYLAMINO)-BUTAN-2-OL

### Step 1

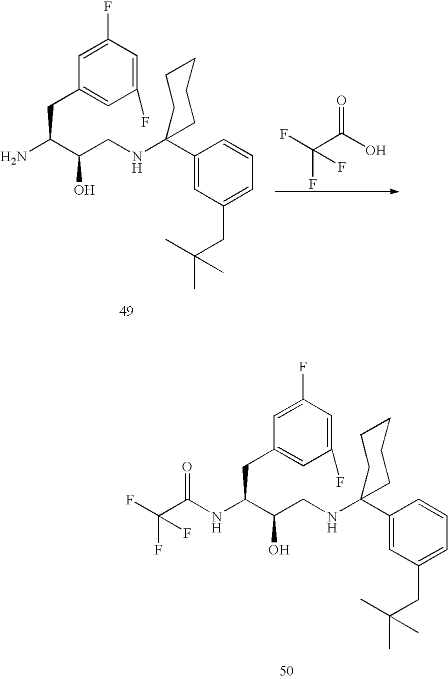

1-[3-(Dimethylamino)propyl]-3-ethylcarbodiimide hydrochloride was added to a THF (anhydrous) solution (500 μL) of amine 49 (0.186 mmol, 80 mg), trifluoroacetic acid (0.186 mmol, 18 mg), diisopropylethylamine (0.386 mmol, 48 mg) and hydroxybenzotriazole (0.2 mmol, 27.6 mg). The reaction was capped and allowed to shake at room temperature for 12 hours at which time LCMS indicated complete reaction. The reaction was evaporated of THF by N₂ stream, acidified with 1 N HCl in ethanol (100 μL), diluted (400 μL ethanol), and filtered. The solution was injected onto a preparative RP-HPLC [Method 10] for purification to provide amide 50. LCMS ret. time (min): 2.77; [M+H]=526.80.

LCMS: Column dimensions: 50 mm(long)×3 mm(i.d.), C-18 stationary phase, 5 micron particle size, 100 angstrom pore size. Mobile phases are 0.05% trifluoroacetic acid in water (solvent A), and 0.05% trifluoroacetic acid in acetonitrile. Chromatographic conditions are 3 mL/min.: 5% solvent B from 0 to 0.275 minutes, 5% to 95% solvent B from 0.275 to 2.75 minutes, then 95% solvent B from 2.75 to 3.50 minutes.

### Step 2

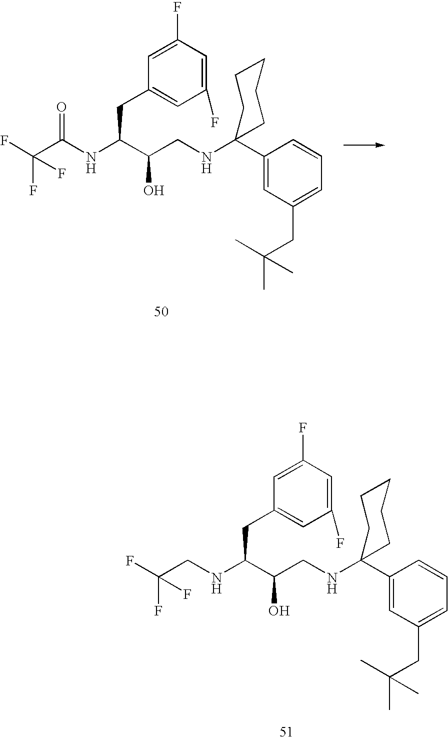

Amide 50 (15 mg, 0.0285 mmol) was dissolved in BH₃ dimethylsulfide complex (2M in THF, 100 μL, 0.2 mmol), and the reaction was capped and heated with shaking at 80° C. for 4 hours. At this time, LCMS was performed showing a complete reaction. The reaction was quenched with a few drops of isopropanol, then evaporated of volatiles by N₂ stream, acidified with 1 N HCl in ethanol (100 μL), diluted (400 μL ethanol), and filtered. This solution was injected onto a preparative RP-HPLC [Method 10] for purification yielding 4-(3,5-Difluoro-phenyl)-1-{1-[3-(2,2-dimethyl-propyl)-phenyl]-cyclohexylamino}-3-(2,2,2-trifluoro-ethylamino)-butan-2-ol (51).

LCMS: Column dimensions: 50 mm(long)×3 mm(i.d.), C-18 stationary phase, 5 micron particle size, 100 angstrom pore size. Mobile phases are 0.05% trifluoroacetic acid in water (solvent A), and 0.05% trifluoroacetic acid in acetonitrile. Chromatographic conditions are 3 mL/min.: 5% solvent B from 0 to 0.275 minutes, 5% to 95% solvent B from 0.275 to 2.75 minutes, then 95% solvent B from 2.75 to 3.50 minutes. Ret. time (min): 2.37; [M+H]=512.90.

### EXAMPLE 321

### SYNTHESIS OF ORTHO-BROMIDE I. STARTING INTERMEDIATE FOR METHOD A (ABOVE)

scheme: synthesis of ortho-bromo intermediate I

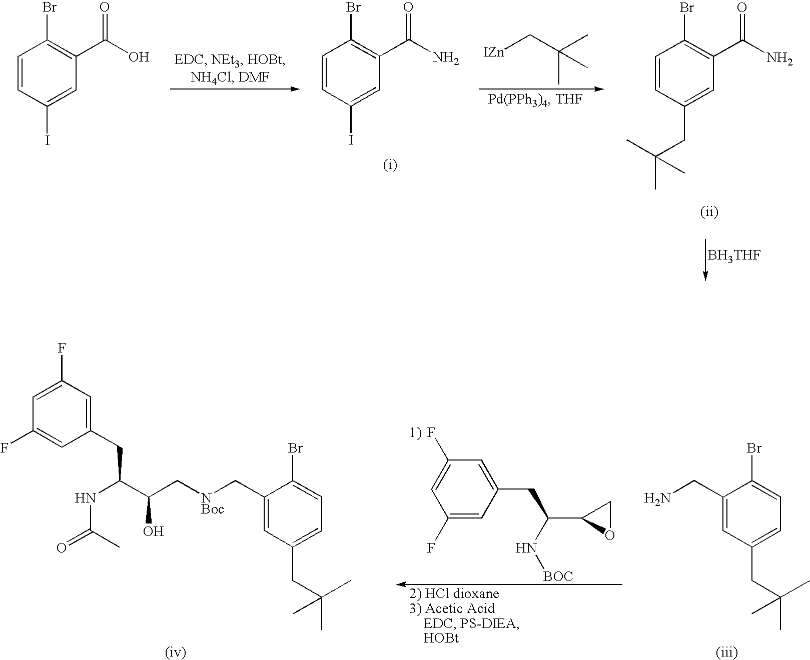

(i). To commercially available 2-bromo-5-iodobenzoic acid (76.5 mmol, 25 g), hydroxybenzotriazole (HOBt, 76.5 mmol, 10.4 g), triethylamine (TEA, 153 mmol, 21.3 mL) and ammonium chloride (84.1 mmol, 4.50 g), is added DMF (anhydrous, 300 mL). After dissolution of solids by stirring, 1-[3-(Dimethylamino)propyl]-3-ethylcarbodiimide hydrochloride (EDC-HCl, 84.1 mmol, 16.08 g) is added. Stirring continues with the reaction capped for 16 h. The reaction is concentrated to half the original volume via roto-evaporation, then 1 L ethyl acetate is added and the subsequent solution is washed once with 1 M HCl (300 mL), then once with saturated NaHCO₃ (300 mL), then twice with H₂O, and then once with saturated NaCl (100 mL). A white solid resulted on drying the ethyl acetate phase with magnesium sulfate, filtering through celite, and evaporation of volitiles.

LCMS (method a see below, retention time at 220 nm detection is 1.51 min and [M=1]⁺=325.8) shows nearly quantitative product (i) at >95% purity.

(ii). To a THF (anhydrous, 300 mL) solution of amide ((i), 76.5 mmol) and tetrakis(triphenylphosphine)palladium(0) (3.825 mmol, 4.42 g), is slowly added neopentylzinc iodide (commercially available 0.5M in THF, 95.6 mmol, 190 mL). The mixture is capped and allowed to stir at 40° C. (in a temperature controlled water bath) for 12 h. The reaction solution is then quenched by adding 1 N HCl in ethanol (100 mL), and then evaporated of volitiles via roto-evaporation. The resulting brown solid mass is partially taken up in ethyl acetate (500 mL), filtered, and the filtrate evaporated of volitiles via roto-evaporation. LCMS (conditions a below) of the crude residue shows a complex mixture, and the product can be purified by passing a concentrated ethyl acetated solution through a silica column with hexanes/ethyl acetate eluent and fractionation. Pure fractions containing (ii) are determined by LCMS (conditions a below, retention 2.19 min, [M+1]=269.83). The pure fractions are evaporated of solvent via roto-evaporation and high vacuum.

(iii). The bromo amide ((ii), 3.7 mmol, 1.0 g) is dissolved in a 2M BH₃ (dimethylsulfide complex) solution in THF (55 mmol, 27.8 mL) then refluxed (reaction flask equipped with a water cooled condenser) for 24 h. At the end of reflux and after cooling, the mixture is quenced with the slow addition of isopropanol (50 mL). The reaction is removed of volitiles via roto-evaporation, the resulting oil is taken up in ethyl acetate (75 mL) and washed once with aqueous HCl (1M, 25 mL), the organic layer is dried with magnesium sulfate, filtered, evaporated via roto-evaporation, and traces of volitiles removed with high vacuum. LCMS (see method a below) of the crude work-up residue shows 85% HPLC pure desired amine (retention 2.17 min, [M+1]=255.67).

(iv). The elaboration of (ii) to (iv) is done similarly to other related compounds in this series (three steps which have been described earlier). The final I is characterized by LCMS (see condition a below, retention 1.93 min, [M+1]=496.98).

LCMS method a. Column dimensions: 50 mm(long)×3 mm(i.d.), C-18 stationary phase, 5 micron particle size, 100 angstrom pore size. Mobile phases are 0.05% trifluoroacetic acid in water (solvent A), and 0.05% trifluoroacetic acid in acetonitrile. Chromatographic conditions are 3 mL/min.: 5% solvent B from 0 to 0.275 min, 5% to 95% solvent B from 0.275 to 2.75 min, then 95% solvent B from 2.75 to 3.50 min.

LCMS method b. This method uses the same column, mobile phases, and flow rate as described above (in LCMS method a). The program gradient is 10% solvent B from 0 to 0.25 min, 10% to 90% solvent B from 0.25 to 9.50 min, then 90% solvent B from 9.50 to 10.25 min.

Preparative LC method. Column dimensions: 150 mm(long)×21.2 mm(i.d.), C-18 staionary phase, 5 micron particle size, 100 angstrom pore size. Mobile phases are 0.1% Trifluoroacetic acid in water (solvent A), and 0.1% trifluoroacetic acid in acetonitrile. Chromatographic conditions are 25 mL/min.: 5% solvent B from 0 to 4.0 min, 5% to 95% solvent B from 4.0 to 22.0 min, 95% solvent B from 22.0 to 24.0 min 95% to 5% solvent B from 24.0 to 24.4 min, then 5% solvent B from 24.4 to 27.0 min.

### EXAMPLE 322

### PREPARATION OF ACETIC AClD 2-AMINO-1-{[1-(3-TERT-BUTYL-PHENYL)-CYCLOHEXYLAMINO]-METHYL}-3-(3,5-DIFLUORO-PHENYL)-PROPYL ESTER

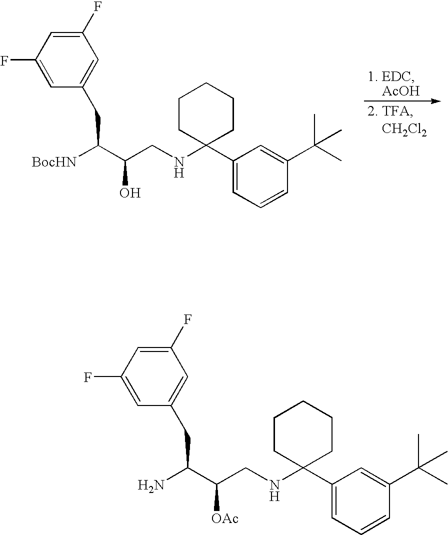

[3-[1-(3-tert-butyl-phenyl)-cyclohexylamino]-1-(3,5-difluoro-benzyl)-2-hydroxy-propyl]-carbamic acid tert-butyl ester (0.23 g, 0.43 mmol) dissolved in CH₂Cl₂ (5 mL) was treated with acetic acid (0.03 mL, 0.50 mmol), NMM (0.2 mL), HOBt (0.068 g, 0.50 mmol) and EDC (0.096 mL, 0.50 mmol) at room temperature, overnight. The Boc protection group was removed with 50% TFA in CH₂Cl₂. Crude product (yield 0.23 g, 0.49 mmol) was used in the next step without purification. Retention time (min)=1.71, method [1]; MS (ESI) 473.5.

### EXAMPLE 323

### PREPARATION OF N-[3-[1-(3-TERT-BUTYL-PHENYL)-CYCLOHEXYLAMINO]-1-(3,5-DIFLUORO-BENZYL)-2-HYDROXY-PROPYL]-METHANESULFONAMIDE

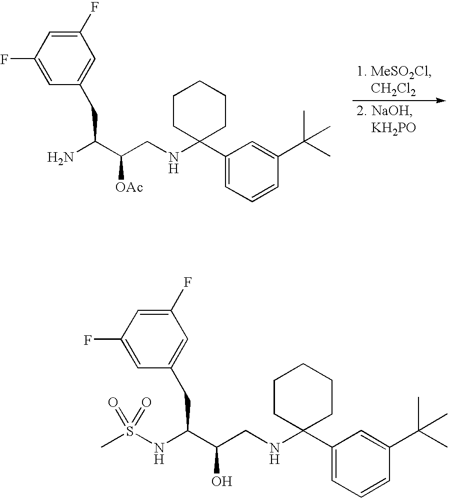

To an ice cold, stirred solution of acetic acid 2-amino-1-{[1-(3-tert-butyl-phenyl)-cyclohexylamino]-methyl}-3-(3,5-difluoro-phenyl)-propyl ester (0.12 g, 0.26 mmol) and Et₃N (0.2 mL) in CH₂Cl₂ (3 mL) was added mesyl chloride (0.02 mL, 0.26 mmol). The reaction mixture was stirred for 30 min and then partitioned between CH₂Cl₂ and water. Organic layer was washed with NaHCO₃, dried and concentrated. Dissolved in MeOH (2 mL) and treated with 2 eq. of 1 N NaOH. After 30 min at room temperature, the reaction mixture was slowly neutralized with aqueous solution of KH₂PO₄. Ethyl acetate extracts were combined and washed with NaHCO₃ and brine, dried and concentrated. Product was purified by HPLC. Retention time (min)=2.04, method [1]; ¹H NMR (300 MHz, DMSO-d₆); δ 9.03 (bs, 1H), 8.65 (bs, 1H), 7.65 (s, 1H), 7.43 (s, 2H), 7.27 (d, J=9 Hz, 1H), 7.07 (t, J=9.3 Hz, 1H), 6.96 (d, J=6.6 Hz, 1H), 5.71 (d, J=5.4 Hz, 1H); 3.62 (bs, 1H), 2.86-2.72 (m, 3H), 2.66-2.50 (m, 4H), 2.28 (s, 3H), 2.04-1.94 (m, 2H), 1.78-1-71 (m, 2H), 1.52 (bs, 1H), 1.30 (s, 9H), 1.25-1.09 (m, 4H); MS (ESI) 509.3.

In an embodiment, the protecting group is t-butoxycarbonyl (Boc) and/or benzyloxycarbonyl (CBZ). In another embodiment, the protecting group is Boc. One skilled in the art will recognize suitable methods of introducing a Boc or CBZ protecting group and may additionally consult Protective Groups in Organic Chemistry, for guidance.

The compounds of the present invention may contain geometric or optical isomers as tautomers. Thus, the present invention includes all tautomers and pure geometric isomers, such as the E and Z geometric isomers, as mixtures thereof. Further, the present invention includes pure enantiomers, diastereomers and/or mixtures thereof, including racemic mixtures. The individual geometric isomers, enantiomers or diastereomers may be prepared or isolated by methods known to those in the art, including, for example chiral chromatography, preparing diastereomers, separating the diastereomers and then converting the diastereomers into enantiomers.

Compounds of the present invention with designated stereochemistry can be included in mixtures, including racemic mixtures, with other enantiomers, diastereomers, geometric isomers or tautomers. In a preferred embodiment, compounds of the present invention are typically present in these mixtures in diastereomeric and/or enantiomeric excess of at least 50%. Preferably, compounds of the present invention are present in these mixtures in diastereomeric and/or enantiomeric excess of at least 80%. More preferably, compounds of the present invention with the desired stereochemistry are present in diastereomeric and/or enantiomeric excess of at least 90 percent. Even more preferably, compounds of the present invention with the desired stereochemistry are present in diastereomeric and/or enantiomeric excess of at least 99%. Preferably the compounds of the present invention have the “S” configuration at position 1. Also preferred are compounds that have the “R” configuration at position 2. Most preferred are compounds that have the “1S,2R” configuration.

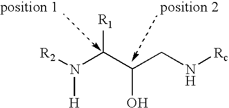

All compound names were generated using AutoNom (AUTOmatic NOMenclature) version 2.1, ACD Namepro version 5.09, Chemdraw Ultra (versions 6.0, 8.0, 8.03, and 9.0), Chemdraw Ultra version 8.0, or were derived therefrom.

Several of the compounds of formula (I) are amines, and as such form salts when reacted with acids. Pharmaceutically acceptable salts are preferred over the corresponding amines since they produce compounds which are more water soluble, stable and/or more crystalline.

### EXAMPLE 324

### BIOLOGICAL EXAMPLES

Properties such as efficacy, oral bioavailability, selectivity, or blood-brain penetration can be assessed by techniques and assays known to one skilled in the art. Exemplary assays for determining such properties are found below.

### Inhibition of APP Cleavage

The methods of treatment and compounds of the present invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While many theories exist, inhibition of beta-secretase activity is thought to inhibit production of A-beta.

Inhibitory activity is demonstrated in one of a variety of inhibition assays, whereby cleavage of an APP substrate in the presence of beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compounds of formula (I) are known. Representative assay systems are described, for example, in U.S. Pat. Nos. 5,942,400, 5,744,346, as well as in the Examples below.

The enzymatic activity of beta-secretase and the production of A-beta can be analyzed in vitro or in vivo, using natural, mutated, and/or synthetic APP substrates, natural, mutated, and/or synthetic enzyme, and the compound employed in the particular method of treatment. The analysis can involve primary or secondary cells expressing native, mutant, and/or synthetic APP and enzyme, animal models expressing native APP and enzyme, or can utilize transgenic animal models expressing the substrate and enzyme. Detection of enzymatic activity can be by analysis of at least one of the cleavage products, for example, by immunoassay, fluorometric or chromogenic assay, HPLC, or other means of detection. Inhibitory compounds are determined as those able to decrease the amount of beta-secretase cleavage product produced in comparison to a control, where beta-secretase mediated cleavage in the reaction system is observed and measured in the absence of inhibitory compounds.

For example, efficacy values yield information regarding a compound's preference for a target tissue by comparing the compound's effect on multiple (i.e., two) tissues. See, for example, Dovey et al., J. Neurochemistry, 2001, 76:173-181. Efficacy reflects the ability of compounds to target a specific tissue and create the desired result (e.g., clinically). Efficacious compositions and corresponding methods of treatment are needed to prevent or treat conditions and diseases associated with amyloidosis.

Efficacious compounds of the present invention are those able to decrease the amount of A-beta produced compared to a control, where beta-secretase mediated cleavage is observed and measured in the absence of the compounds. Detection of efficacy can be by analysis of A-beta levels, for example, by immunoassay, fluorometric or chromogenic assay, HPLC, or other means of detection. The efficacy of the compounds of formula (I) was determined as a percentage inhibition corresponding to A-beta concentrations for tissue treated and untreated with a compound of formula (I).

### Beta-Secretase

Various forms of beta-secretase enzyme are known, are available, and useful for assaying of enzymatic activity and inhibition of enzyme activity. These include native, recombinant, and synthetic forms of the enzyme. Human beta-secretase is known as Beta Site APP Cleaving Enzyme (BACE), BACE1, Asp2, and memapsin 2, and has been characterized, for example, in U.S. Pat. No. 5,744,346 and published PCT patent applications WO 98/22597, WO 00/03819, WO 01/23533, and WO 00/17369, as well as in literature publications (Hussain et al., 1999, Mol. Cell. Neurosci., 14:419-427; Vassar et al., 1999, Science, 286:735-741; Yan et al., 1999, Nature, 402:533-537; Sinha et al., 1999, Nature, 40:537-540; and Lin et al., 2000, Proceedings Natl. Acad. Sciences USA, 97:1456-1460). Synthetic forms of the enzyme have also been described in, for example (WO 98/22597 and WO 00/17369). Beta-secretase can be extracted and purified from human brain tissue and can be produced in cells, for example mammalian cells expressing recombinant enzyme.

### APP Substrate

Assays that demonstrate inhibition of beta-secretase-mediated cleavage of APP can utilize any of the known forms of APP, including the 695 amino acid “normal” isotype described by Kang et al., 1987, Nature, 325:733-6, the 770 amino acid isotype described by Kitaguchi et. al., 1981, Nature, 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK, disclosed, for example, in WO 00/17369, fragments of APP, and synthetic peptides containing the beta-secretase cleavage site, wild type (WT) or mutated form, (e.g., SW), as described, for example, in U.S. Pat. No. 5,942,400 and WO 00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety can be an antigenic epitope for antibody binding, a label or other detection moiety, a binding substrate, and the like.

### Antibodies

Products characteristic of APP cleavage can be measured by immunoassay using various antibodies, as described, for example, in Pirttila et al., 1999, Neuro. Lett., 249:21-4, and in U.S. Pat. No. 5,612,486. Useful antibodies to detect A-beta include, for example, the monoclonal antibody 6E10 (Senetek, St. Louis, Mo.) that specifically recognizes an epitope on amino acids 1-16 of the A-beta peptide, antibodies 162 and 164 (New York State Institute for Basic Research, Staten Island, N.Y.) that are specific for human A-beta 1-40 and 1-42, respectively, and antibodies that recognize the junction region of A-beta, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

### Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

### Cell Free Assays

Exemplary assays that can be used to demonstrate the inhibitory activity of the compounds of the present invention are described, for example, in WO 00/17369, WO 00/03819, and U.S. Pat. Nos. 5,942,400 and 5,744,346. Such assays can be performed in cell-free incubations or in cellular incubations using cells expressing beta-secretase and an APP substrate having a beta-secretase cleavage site.

An APP substrate containing the beta-secretase cleavage site of APP, for example, a complete APP or variant, an APP fragment, or a recombinant or synthetic APP substrate containing the amino acid sequence KM-DA or NL-DA is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that can be fusion proteins or peptides that contain the substrate peptide and a modification useful to facilitate the purification or detection of the peptide or its beta-secretase cleavage products. Useful modifications include the insertion of a known antigenic epitope for antibody binding, the linking of a label or detectable moiety, the linking of a binding substrate, and the like.

Suitable incubation conditions for a cell-free in vitro assay include, for example, approximately 200 nM to 10 μM substrate, approximately 10 pM to 200 pM enzyme, and approximately 0.1 nM to 10 μM inhibitor compound, in aqueous solution, at an approximate pH of 4-7, at approximately 37° C., for a time period of approximately 10 min to 3 h. These incubation conditions are exemplary only, and canvary as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by an anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

### Cellular Assay

Numerous cell-based assays can be used to analyze beta-secretase activity and/or processing of APP to release A-beta. Contact of an APP substrate with a beta-secretase enzyme within the cell and in the presence or absence of a compound inhibitor of the present invention can be used to demonstrate beta-secretase inhibitory activity of the compound. It is preferred that the assay in the presence of a useful inhibitory compound provides at least about 10% inhibition of the enzymatic activity, as compared with a non-inhibited control.

In an embodiment, cells that naturally express beta-secretase are used. Alternatively, cells are modified to express a recombinant beta-secretase or synthetic variant enzyme as discussed above. The APP substrate can be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A-beta from APP provide useful means to assay inhibitory activities of the compounds employed in the methods of treatment of the present invention. Production and release of A-beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of at least one cleavage product of the APP substrate. For example, inhibition of beta-secretase activity against the substrate APP would be expected to decrease the release of specific beta-secretase induced APP cleavage products such as A-beta.

Although both neural and non-neural cells process and release A-beta, levels of endogenous beta-secretase activity are low and often difficult to detect by EIA. The use of cell types known to have enhanced beta-secretase activity, enhanced processing of APP to A-beta, and/or enhanced production of A-beta are therefore preferred. For example, transfection of cells with the Swedish Mutant form of APP (APP-SW), with APP-KK, or with APP-SW-KK provides cells having enhanced beta-secretase activity and producing amounts of A-beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor employed in the methods of treatment, the amount of A-beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

### In Vivo Assays: Animal Models

Various animal models can be used to analyze beta-secretase activity and/or processing of APP to release A-beta, as described above. For example, transgenic animals expressing APP substrate and beta-secretase enzyme can be used to demonstrate inhibitory activity of the compounds of the present invention. Certain transgenic animal models have been described, for example, in U.S. Pat. Nos. 5,877,399, 5,612,486, 5,387,742, 5,720,936, 5,850,003, 5,877,015, and 5,811,633, and in Games et al., 1995, Nature, 373:523. Animals that exhibit characteristics associated with the pathophysiology of Alzheimer's disease are preferred. Administration of the compounds of the present invention to the transgenic mice described herein provides an alternative method for demonstrating the inhibitory activity of the compounds. Administration of the compounds of the present invention in a pharmaceutically effective carrier and via an administrative route that reaches the target tissue in an appropriate therapeutic amount is also preferred.

Inhibition of beta-secretase mediated cleavage of APP at the beta-secretase cleavage site and of A-beta release can be analyzed in these animals by measuring cleavage fragments in the animal's body fluids such as cerebral fluid or tissues. Analysis of brain tissues for A-beta deposits or plaques is preferred.

### A: Enzyme Inhibition Assay

The methods of treatment and compounds of the present invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of MBP and the carboxy terminal 125 amino acids of APP-SW, the Swedish mutation. The beta-secretase enzyme is derived from human brain tissue as described in Sinha et al., 1999, Nature, 40:537-540 or recombinantly produced as the full-length enzyme (amino acids 1-501), and can be prepared, for example, from 293 cells expressing the recombinant cDNA, as described in WO 00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu596 at the substrate's APP-SW 751 mutation site.

### Specific Asssay Procedure

Compounds of formula (I) are diluted in a 1:1 dilution series to a six-point concentration curve (two wells per concentration) in one row of a 96-well plate per compound tested. Each of the test compounds is prepared in DMSO to make up a 10 mM stock solution. The stock solution is serially diluted in DMSO to obtain a final compound concentration of 200 μM at the high point of a 6-point dilution curve. Ten (10) μL of each dilution is added to each of two wells on row C of a corresponding V-bottom plate to which 190 μL of 52 mM NaOAc, 7.9% DMSO, pH 4.5 are pre-added. The NaOAc diluted compound plate is spun down to pellet precipitant and 20 μL/well is transferred to a corresponding flat-bottom plate to which 30 μL of ice-cold enzyme-substrate mixture (2.5 μL MBP-C125SW substrate, 0.03 μL enzyme and 24.5 μL ice cold 0.09% TX100 per 30 μL) is added. The final reaction mixture of 200 μM compound at the highest curve point is in 5% DMSO, 20 μM NaOAc, 0.06% TX100, at pH 4.5.

Warming the plates to 37° C. starts the enzyme reaction. After 90 min at 37° C., 200 μL/well cold specimen diluent is added to stop the reaction and 20 μL/well was transferred to a corresponding anti-MBP antibody coated ELISA plate for capture, containing 80 μL/well specimen diluent. This reaction is incubated overnight at 4° C. and the ELISA is developed the next day after a 2 h incubation with anti-192SW antibody, followed by Streptavidin-AP conjugate and fluorescent substrate. The signal is read on a fluorescent plate reader.

Relative compound inhibition potency is determined by calculating the concentration of compound that showed a 50% reduction in detected signal (IC₅₀) compared to the enzyme reaction signal in the control wells with no added compound. In this assay, preferred compounds of the present invention exhibit an IC₅₀ of less than 50 μM.

### B: FP Bace Assay

### Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds employed in the present invention. Useful substrates include
Biotin-SEVN L-DAEFRC[oregon green]KK,
Biotin-SEVKM-DAEFRC[oregon green]KK,
Biotin-GLNIKTEEISEISY-EVEFRC[oregon green]KK,
Biotin-ADRGLTTRPGSGLTNIKTEEISEVNL-DAEFRC[oregon green]KK, and
Biotin-FVNQHLCoxGSHLVEALY-LVCoxGERGFFYTPKAC[oregon green]KK.

The enzyme (0.1 nM) and test compounds (0.001-100 μM) are incubated in pre-blocked, low affinity, black plates (384 well) at 37° C. for 30 min. The reaction is initiated by addition of 150 mM substrate to a final volume of 30 μL/well. The final assay conditions are 0.001-100 μM compound inhibitor, 0.1 molar sodium acetate (pH 4.5), 150 nM substrate, 0.1 nM soluble beta-secretase, 0.001% Tween 20, and 2% DMSO. The assay mixture is incubated for 3 h at 37° C., and the reaction is terminated by the addition of a saturating concentration of immunopure streptavidin. After incubation with streptavidin at room temperature for 15 min, fluorescence polarization is measured, for example, using a LJL Acqurest (Ex485 nm/Em530 nm).

The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate. In this assay, preferred compounds of the present invention exhibit an IC₅₀ of less than 50 μM. More preferred compounds of the present invention exhibit an IC₅₀ of less than 10 μM. Even more preferred compounds of the present invention exhibit an IC₅₀ of less than 5 μM.

### C: Beta-Secretase Inhibition: P26-P4′ SW Assay

Synthetic substrates containing the beta-secretase cleavage site of APP are used to assay beta-secretase activity, using the methods described, for example, in published PCT application WO 00/47618. The P26-P4′ SW substrate is a peptide of the sequence (biotin)CGGADRGLTTRPGSGLTNIKTEEISEVNLDAEF. The P26-P1 standard has the sequence (biotin)CGGADRGLTTRPGSGLTNIKTEEISEVNL.

Briefly, the biotin-coupled synthetic substrates are incubated at a concentration of from about 0 to about 200 μM in this assay. When testing inhibitory compounds, a substrate concentration of about 1.0 μM is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 to 2000 pM, after dilution.

The reaction mixture also includes 20 mM sodium acetate, pH 4.5, 0.06% Triton X100, and is incubated at 37° C. for about 1 to 3 h. Samples are then diluted in assay buffer (for example, 145.4 nM sodium chloride, 9.51 mM sodium phosphate, 7.7 mM sodium azide, 0.05% Triton X405, 6 g/L bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 h at 4° C. After washing in TTBS buffer (150 mM sodium chloride, 25 mM Tris, 0.05% Tween 20, pH 7.5), the samples are incubated with streptavidin-AP according to the manufacturer's instructions. After a 1 h incubation at room temperature, the samples are washed in TTBS and incubated with fluorescent substrate solution A (31.2 g/L 2-amino-2-methyl-1-propanol, 30 mg/L, pH 9.5). Reaction with streptavidin-alkaline phosphate permits detection by fluorescence. Compounds that are effective inhibitors of beta-secretase activity demonstrate reduced cleavage of the substrate as compared to a control.

### D: Assays using Synthetic Oligopeptide-Substrates

Synthetic oligopeptides are prepared incorporating the known cleavage site of beta-secretase, and optionally include detectable tags, such as fluorescent or chromogenic moieties. Examples of such peptides, as well as their production and detection methods, are described in U.S. Pat. No. 5,942,400. Cleavage products can be detected using high performance liquid chromatography, or fluorescent or chromogenic detection methods appropriate to the peptide to be detected, according to methods well known in the art.

By way of example, one such peptide has the sequence SEVNL-DAEF, and the cleavage site is between residues 5 and 6. Another preferred substrate has the sequence ADRGLTTRPGSGLTNIKTEEISEVNL-DAEF, and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of a compound inhibitor to control results provides a measure of the compound's inhibitory activity.

### E: Inhibition of Beta-Secretase Activity-Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met652 to Asn651Leu652 (numbered for APP751), commonly called the Swedish mutation and shown to overproduce A-beta (Citron et al., 1992, Nature, 360:672-674), as described in U.S. Pat. No. 5,604,102.

The cells are incubated in the presence/absence of the inhibitory compound (diluted in DMSO) at the desired concentration, generally up to 10 μg/mL. At the end of the treatment period, conditioned media is analyzed for beta-secretase activity, for example, by analysis of cleavage fragments. A-beta can be analyzed by immunoassay, using specific detection antibodies. The enzymatic activity is measured in the presence and absence of the compound inhibitors to demonstrate specific inhibition of beta-secretase mediated cleavage of APP substrate.

### F: Inhibition of Beta-Secretase in Animal Models of Alzheimer's Disease

Various animal models can be used to screen for inhibition of beta-secretase activity. Examples of animal models useful in the present invention include mouse, guinea pig, dog, and the like. The animals used can be wild type, transgenic, or knockout models. In addition, mammalian models can express mutations in APP, such as APP695-SW and the like as described herein. Examples of transgenic non-human mammalian models are described in U.S. Pat. Nos. 5,604,102, 5,912,410 and 5,811,633.

PDAPP mice, prepared as described in Games et al., 1995, Nature, 373:523-527 are useful to analyze in vivo suppression of A-beta release in the presence of putative inhibitory compounds. As described in U.S. Pat. No. 6,191,166, 4-month-old PDAPP mice are administered a compound of formula (I) formulated in a vehicle, such as corn oil. The mice are dosed with the compound (1-30 mg/mL, preferably 1-10 mg/mL). After a designated time, e.g., 3-10 h, the brains are analyzed.

Transgenic animals are administered an amount of a compound formulated in a carrier suitable for the chosen mode of administration. Control animals are untreated, treated with vehicle, or treated with an inactive compound. Administration can be acute, (i.e. single dose or multiple doses in one day), or can be chronic, (i.e. dosing is repeated daily for a period of days). Beginning at time 0, brain tissue or cerebral fluid is obtained from selected animals and analyzed for the presence of APP cleavage peptides, including A-beta, for example, by immunoassay using specific antibodies for A-beta detection. At the end of the test period, animals are sacrificed and brain tissue or cerebral fluid is analyzed for the presence of A-beta and/or beta-amyloid plaques. The tissue is also analyzed for necrosis.

Reduction of A-beta in brain tissues or cerebral fluids and reduction of beta-amyloid plaques in brain tissue are assessed by administering the compounds of formula (I), or pharmaceutical compositions comprising compounds of formula (I) to animals and comparing the data with that from non-treated controls.

### G: Inhibition of A-beta Production in Human Patients

Patients suffering from Alzheimer's disease demonstrate an increased amount of A-beta in the brain. Alzheimer's disease patients are subjected to a method of treatment of the present invention, (i.e. administration of an amount of the compound inhibitor formulated in a carrier suitable for the chosen mode of administration). Administration is repeated daily for the duration of the test period. Beginning on day 0, cognitive and memory tests are performed, for example, once per month.

Patients administered the compounds of formula (I) are expected to demonstrate slowing or stabilization of disease progression as analyzed by a change in at least one of the following disease parameters: A-beta present in cerebrospinal fluid or plasma; brain or hippocampal volume; A-beta deposits in the brain; amyloid plaque in the brain; or scores for cognitive and memory function, as compared with control, non-treated patients.

### H: Prevention of A-beta Production in Patients at Risk for Alzheimer's Disease

Patients predisposed or at risk for developing Alzheimer's disease can be identified either by recognition of a familial inheritance pattern, for example, presence of the Swedish Mutation, and/or by monitoring diagnostic parameters. Patients identified as predisposed or at risk for developing Alzheimer's disease are administered an amount of the compound inhibitor formulated in a carrier suitable for the chosen mode of administration. Administration is repeated daily for the duration of the test period. Beginning on day 0, cognitive and memory tests are performed, for example, once per month.

Patients subjected to a method of treatment of the present invention (i.e., administration of at least one compound of formula (I)) are expected to demonstrate slowing or stabilization of disease progression as analyzed by a change in at least one of the following disease parameters: A-beta present in cerebrospinal fluid or plasma; brain or hippocampal volume; amyloid plaque in the brain; or scores for cognitive and memory function, as compared with control, non-treated patients.

### I: Efficacy of Compounds to Inhibit A-beta Concentration

The invention encompasses compounds of formula (I) that are efficacious. Efficacy is calculated as a percentage of concentrations as follows:
Efficacy=(1−(total A-beta in dose group/total A-beta in vehicle control)*100%
wherein the “total A-beta in dose group” equals the concentration of A-beta in the tissue, (e.g., rat brain) treated with the compound, and the “total A-beta in vehicle control” equals the concentration of A-beta in the tissue, yielding a % inhibition of A-beta production. Statistical significance is determined by p-value <0.05 using the Mann Whitney t-test. See, for example, Dovey et al., J. Neurochemistry, 2001, 76:173-181.

### J: Selectivity of Compounds for Inhibiting BACE over CatD

The compounds of formula (I) can be selective for beta-secretase versus catD. Wherein the ratio of catD:beta-secretase is greater than 1, selectivity is calculated as follows:
Selectivity=(IC₅₀ for catD/IC₅₀ for beta-secretase)*100%
wherein IC₅₀ is the concentration of compound necessary to decrease the level of catD or beta-secretase by 50%.

The compounds of formula (I) can be selective for beta-secretase versus catE. Wherein the ratio of catE:beta-secretase is greater than 1, selectivity is calculated as follows:
Selectivity=(IC₅₀ for catE/IC₅₀ for beta-secretase)*100%
wherein IC₅₀ is the concentration of compound necessary to decrease the level of catE or beta-secretase by 50%. Selectivity is reported as the ratio of IC₅₀(catE):IC₅₀(BACE).

Pharmacokinetic parameters were calculated by a non-compartmental approach. See, for example, Gibaldi, M. and Perrier, D., Pharmacokinetics, Second Edition, 1982, Marcel Dekker Inc., New York, N.Y., pp 409-418.

Where indicated, diastereomers were separated by reverse phase HPLC using the noted methods. The first isomer collected in each case was designated Diastereomer A, and the second isomer Diastereomer B. Unless otherwise indicated, specific formula (I) compound examples represent mixtures of diastereomers.

### EXAMPLE 325

### EXEMPLARY FORMULA (I) SELECTIVITY COMPOUNDS

In the following examples, each value is an average of four experimental runs and multiple values for one compoundd indicate that more than one experiment was conducted.

### A. CatD/BACE Selectivity of Exemplary Formula (I) Compounds

| | | IC₅₀ CatD/ |
|---|---|---|
| Example No. | Compound | IC₅₀ BACE |
| 325A-1 | 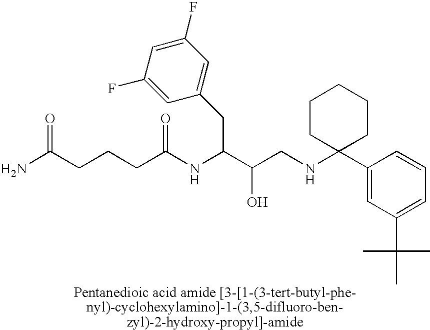 | 3.1 0.9 |
| 325A-2 | 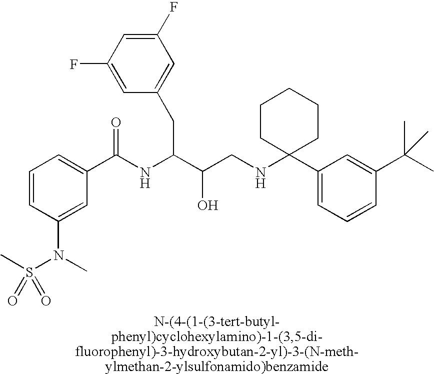 | 3.1 |
| 325A-3 | 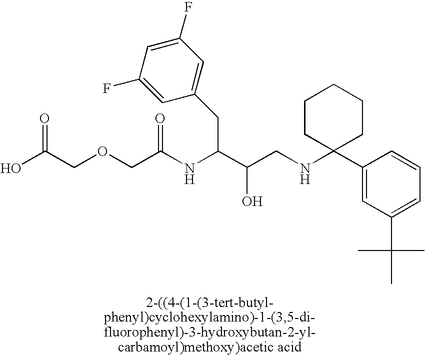 | 2.0 |
| 325A-4 | 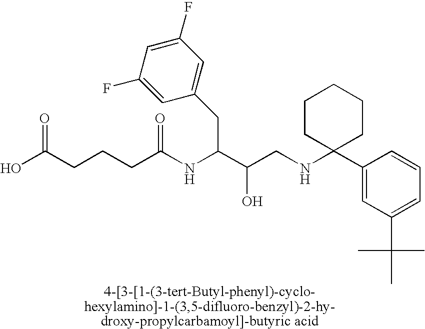 | 1.9 |
| 325A-5 | 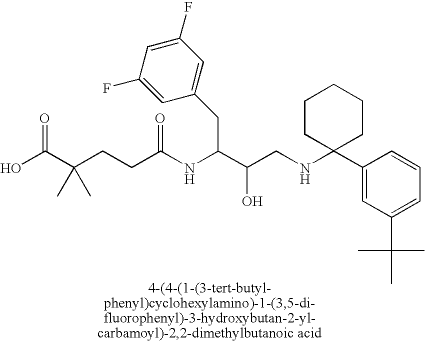 | 1.7 |
| 325A-6 | 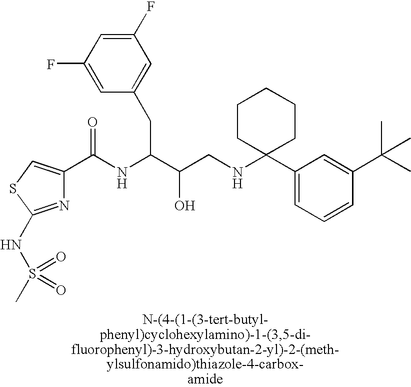 | 5.0 |

### B. CatE/BACE Selectivity of Exemplary Formula (I) Compounds

| | | IC₅₀ CatE/ |
|---|---|---|
| Example No. | Compound | IC₅₀ BACE |
| 325B-1 | 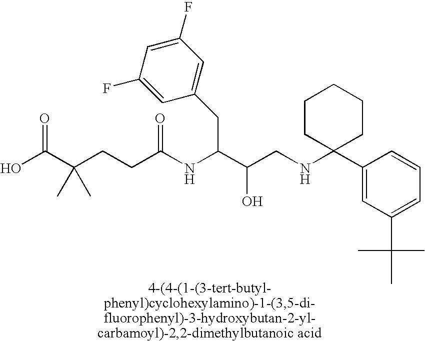 | 8.3 |
| 325B-2 | 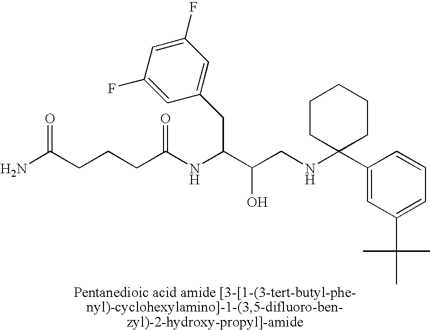 | 5.7 0.5 |
| 325B-3 | 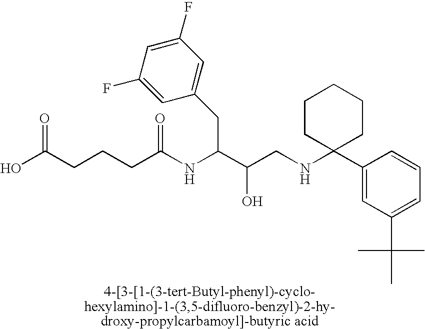 | 3.4 |
| 325B-4 | 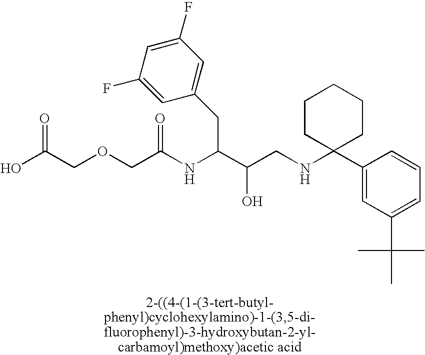 | 3.2 |
| 325B-5 | 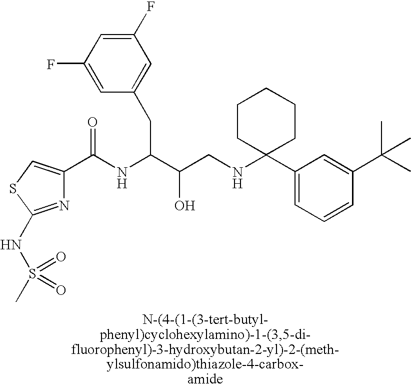 | 1.2 |

### K: Oral Bioavailability of Compounds for Inhibiting Amyloidosis

The invention encompasses compounds of formula (I) that are orally bioavailable. Generally, oral bioavailability is defined as the fraction of orally administered dose reaching systemic circulation. Oral bioavailability can be determined following both an intravenous (IV) and oral (PO) administration of a test compound.

Oral bioavailability was determined in the male Sprague-Dawley rat following both IV and PO administration of test compound. Two month-old male rats (250-300 g) were surgically implanted with polyethylene (PE-50) cannula in the jugular vein while under isoflurane anesthesia the day before the in-life phase. Animals were fasted overnight with water ad libitum, then dosed the next day. The dosing regime consisted of either a 5 mg/kg (2.5 mL/kg) IV dose (N=3) administered to the jugular vein cannula, then flushed with saline, or a 10 mg/kg (5 mL/kg) PO dose (N=3) by esophageal gavage. Compounds were formulated with 10% Solutol in 5% dextrose at 2 mg/mL. Subsequent to dosing, blood was collected at 0.016 (IV only), 0.083, 0.25, 0.5, 1, 3, 6, 9 and 24 h post administration and heparinized plasma was recovered following centrifugation.

Compounds were extracted from samples following precipitation of the plasma proteins by methanol. The resulting supernatants were evaporated to dryness and reconstituted with chromatographic mobile phase (35% acetonitrile in 0.1% formic acid) and injected onto a reverse phase C₁₈ column (2×50 mm, 5 μm, BDS Hypersil). Detection was facilitated with a multi-reaction-monitoring experiment on a tandem triple quadrupole mass spectrometer (LC/MS/MS) following electrospray ionization. Experimental samples were compared to calibration curves prepared in parallel with aged match rat plasma and quantitated with a weighted 1/×linear regression. The lower limit of quantization (LOQ) for the assay was typically 0.5 ng/mL.

Oral bioavailability (% F) is calculated from the dose-normalized ratio of plasma exposure following oral administration to the intravenous plasma exposure in the rat by the following equation
% F=(AUCₚₒ/AUCᵢᵥ)×(Dᵢᵥ/Dₚₒ)×100%
where D is the dose and AUC is the area-under-the-plasma-concentration-time-curve from 0 to 24 h. AUC is calculated from the linear trapezoidal rule by AUC=((C₂+C₁)/2)×(T₂−T₁) where C is concentration and T is time.

Pharmacokinetic parameters were calculated by a non-compartmental approach. See, for example, Gibaldi, M. and Perrier, D., Pharmacokinetics, Second Edition, 1982, Marcel Dekker Inc., New York, N.Y., pp 409-418.L: Brain Uptake

The invention encompasses beta-secretase inhibitors that can readily cross the blood-brain barrier. Factors that affect a compound's ability to cross the blood-brain barrier include a compound's molecular weight, Total Polar Surface Area (TPSA), and log P (lipophilicity). See, e.g., Lipinski, C. A., et al., Adv. Drug Deliv. Reviews, 23:3-25 (1997). One of ordinary skill in the art will be aware of methods for determining characteristics allowing a compound to cross the blood-brain barrier. See, for example, Murcko et al., Designing Libraries with CNS Activity, J. Med. Chem., 42 (24), pp. 4942-51 (1999). Calculations of logP values were performed using the Daylight clogP program (Daylight Chemical Information Systems, Inc.). See, for example, Hansch, C., et al., Substitutent Constants for Correlation Analysis in Chemistry and Biology, Wiley, New York (1979); Rekker, R., The Hydrophobic Fragmental Constant, Elsevier, Amsterdam (1977); Fujita, T., et al., J. Am. Chem. Soc., 86, 5157 (1964). TPSA was calculated according to the methodology outlined in Ertl, P., et al., J. Med. Chem., 43:3714-17 (2000).

The following assay was employed to determine the brain penetration of compounds encompassed by the present invention.

In-life phase: Test compounds were administered to CF-1 (20-30 g) mice at 10 μmol/kg (4 to 7 mg/kg) following IV administration in the tail vein. Two time-points, 5 and 60 min, were collected post dose. Four mice were harvested for heparinized plasma and non-perfused brains at each time-point for a total of 8 mice per compound.

Analytical phase: Samples were extracted and evaporated to dryness, then reconstituted and injected onto a reverse phase chromatographic column while monitoring the effluent with a triple quadrupole mass spectrometer. Quantitation was then performed with a 1/×² weighted fit of the least-squares regression from calibration standards prepared in parallel with the in vivo samples. The lower limit of quantitation (LOQ) is generally 1 ng/mL and 0.5 ng/g for the plasma and brain respectively. Data was reported in micromolar (μM) units. Brain levels were corrected for plasma volumes (16 μL/g).

Results: Comparison of a compound's brain concentration level to two marker compounds, Indinavir and Diazepam, demonstrates the ability in which the compounds of the present invention can cross the blood-brain barrier. Indinavir (HIV protease inhibitor) is a poor brain penetrant marker and Diazepam is a blood flow limited marker. The concentration levels of Indinavir in the brain at 5 and 60 min were 0.165 μM and 0.011 μM, respectively. The concentration levels of Diazepam at 5 and 60 min were 5.481 μM and 0.176 μM, respectively.

Where indicated, specific formula (I) compound examples represent single diastereomers (e.g., diastereomer A).

### EXAMPLE 326

### BRAIN UPTAKE FOR EXEMPLARY FORMULA (I) COMPOUNDS

| Example No. | Compound | [Brain]/[Plasma] | logP | TPSA |
|---|---|---|---|---|
| 326-1 | 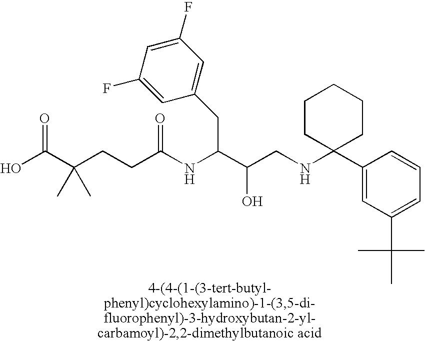 | — | 5.1 | 99 |
| 326-2 | 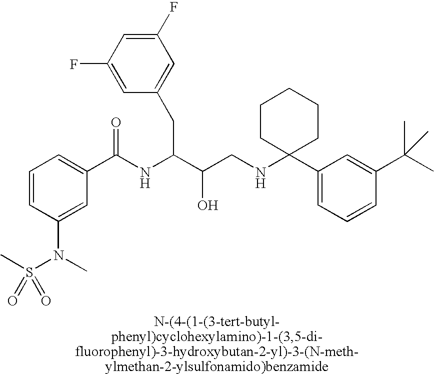 | — | 6.84 | 98.74 |
| 326-3 | 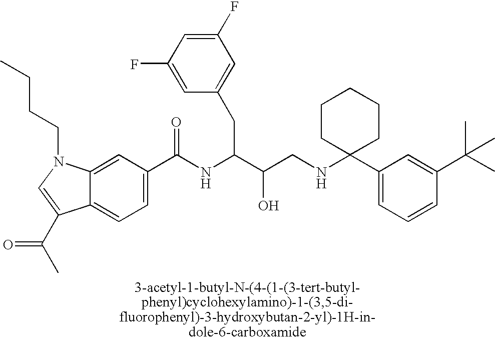 | — | — | 83.36 |

The present invention has been described with reference to various specific and preferred embodiments and techniques. However, it should be understood that many variations and modifications may be made while remaining within the spirit and scope of the present invention.

Unless defined otherwise, all scientific and technical terms used herein have the same meaning as commonly understood by one of skill in the art to which this invention belongs. Although methods and materials similar or equivalent to those described herein can be used in the practice or testing of the present invention, suitable methods and materials are described above. Additionally, the materials, methods, and examples are illustrative only and not intended to be limiting. All publications, patent applications, patents, and other references mentioned herein are incorporated by reference in their entirety. In case of conflict, the present specification, including definitions, will control.

1. A method of preventing or treating at least one condition which benefits from inhibition of at least one aspartyl-protease, comprising:
administering to a host a composition comprising a therapeutically effective amount of at least one selective compound of formula (I), 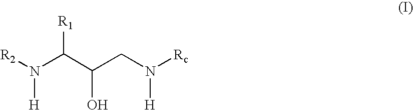
or at least one pharmaceutically acceptable salt thereof, wherein
R₁ is selected from 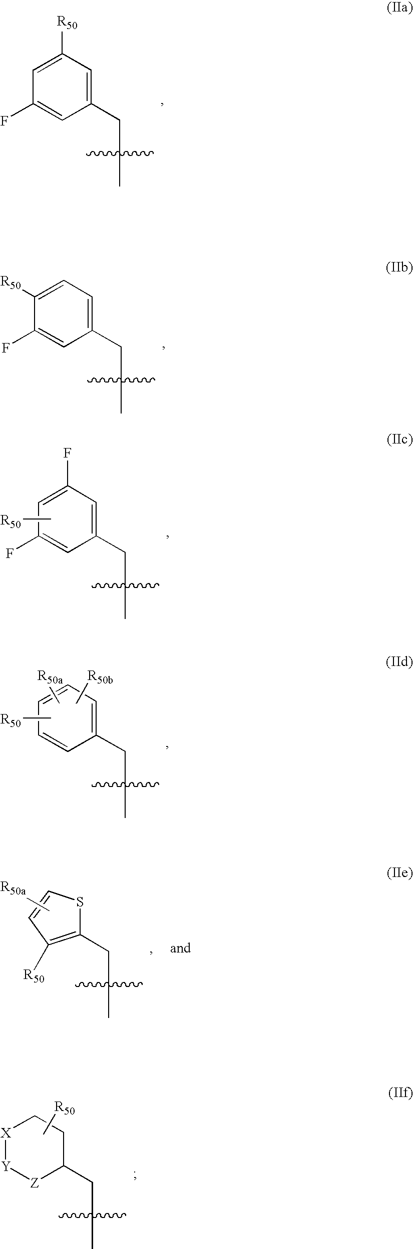
wherein
X, Y, and Z are independently selected from
—C(H)₀₋₂—,
—O—,
—C(O)—,
—NH—, and
—N—;
wherein at least one bond of the (IIf) ring may optionally be a double bond;
R₅₀, R₅₀ₐ, and R_{50b} are independently selected from
—H,
-halogen,
—OH,
—SH,
—CN,
—C(O)-alkyl,
—NR₇R₈,
—NO₂,
—S(O)₀₋₂-alkyl,
-alkyl,
-alkoxy,
—O-benzyl optionally substituted with at least one group independently selected from —H, —OH, and alkyl,
—C(O)—NR₇R₈,
-alkyloxy,
-alkoxyalkoxyalkoxy, and
-cycloalkyl;
wherein the alkyl, alkoxy, and cycloalkyl groups within R₅₀, R₅₀ₐ, and R_{50b} are optionally substituted with at least one group independently selected from alkyl, halogen, OH, —NR₅R₆, —CN, haloalkoxy, —NR₇R₈, and alkoxy;
R₅ and R₆ are independently selected from —H and alkyl, or
R₅ and R₆, and the nitrogen to which they are attached, form a 5 or 6 membered heterocycloalkyl ring; and
R₇ and R₈ are independently selected from
—H,
-alkyl optionally substituted with at least one group independently selected from —OH, —NH₂, and halogen,
-cycloalkyl, and
-alkyl-O-alkyl; 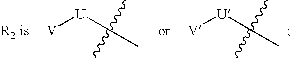
U is selected from —C(O)—, —C(═S)—, —S(O)₀₋₂—, —C(═N—R₂₁)—, —C(═N—OR₂₁)—, —C(O)—NR₂₀—, —C(O)—O—, —S(O)₂—NR₂₀—, and —S(O)₂—O—;
U′ is selected from —C(O)—, —C(═N—R₂₁)—, —C(═N—OR₂₁)—, —C(O)—NR₂₀—, and —C(O)—O—;
V is selected from aryl, heteroaryl, cycloalkyl, heterocycloalkyl, —[C(R₄)(R_{4′})]₁₋₃-D, and -(T)₀₋₁-RN;
V′ is selected from -(T)₀₋₁-R_{N′};
wherein the aryl, heteroaryl, cycloalkyl and heterocycloalkyl groups included within V and V′ are optionally substituted with at least one independently selected R_{B} group;
wherein at least one carbon of the aryl, heteroaryl, cycloalkyl, and heterocycloalkyl groups included within V and V′ are optionally replaced with —N—, —O—, —NH—, —C(O)—, —C(S)—, —C(═N—H)—, —C(═N—OH)—, —C(═N-alkyl)-, or —C(═N—O—alkyl)-;
R_{B} at each occurrence is independently selected from halogen, —OH, —CF₃, —OCF₃, —O-aryl, —CN, —NR₁₀₁R′₁₀₁, alkyl, alkoxy, —(CH₂)₀₋₄—(C(O))₀₋₁—(O)₀₋₁-alkyl, —C(O)—OH, —(CH₂)₀₋₃-cycloalkyl, aryl, heteroaryl, and heterocycloalkyl;
wherein the alkyl, alkoxy, cycloalkyl, aryl, heteroaryl, or heterocycloalkyl groups included within R_{B} are optionally substituted with 1 or 2 groups independently selected from —C₁-C₄ alkyl, —C₁-C₄ alkoxy, —C₁-C₄ haloalkyl, —C₁-C₄ haloalkoxy, halogen, —OH, —CN, and —NR₁₀₁R′₁₀₁;
R₁₀₁ and R′₁₀₁ are independently selected from —H, alkyl, —(C(O))₀₋₁—(O)₀₋₁-alkyl, —C((O))₀₋₁—OH, and aryl;
R₄ and R_{4′} are independently selected from hydrogen, —OH, alkyl, —(CH₂)₀₋₃-cycloalkyl, —(CH₂)₀₋₃OH, fluorine, —CF₃, —OCF₃, —O-aryl, alkoxy, —C₃-C₇ cycloalkoxy, aryl, and heteroaryl, or
R₄ and R_{4′} are taken together with the carbon to which they are attached to form a 3, 4, 5, 6, or 7 membered carbocylic ring wherein 1, 2, or 3 carbons of the ring is optionally replaced with O, —N(H)—, —N(alkyl)-, —N(aryl)-, —C(O)—, or —S(O)₀₋₂;
D is selected from aryl, heteroaryl, cycloalkyl, and heterocycloalkyl; wherein the aryl, heteroaryl, cycloalkyl, and heterocycloalkyl are optionally substituted with 1 or 2 R_{B} groups; and
T is selected from —NR₂₀— and —O—;
R₂₀ is selected from H, —CN, alkyl, haloalkyl, and cycloalkyl;
R₂₁ is selected from —H, alkyl, haloalkyl, and cycloalkyl;
R_{N} is selected from —OH, —NH₂, —NH(alkyl), —NH(cycloalkyl), —N(alkyl)(alkyl), —N(alkyl)(cycloalkyl), —N(cycloalkyl)(cycloalkyl), —R′₁₀₀, alkyl-R₁₀₀, —(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, —(CRR′)₁₋₆—NR₁₀₀—R′₁₀₀, —(CRR′)₁₋₆—P(O)(O-alkyl)₂, alkyl-O-alikyl-C(O)OH, and —CH(R_{E1})—(CH₂)₀₋₃-E₁-E₂-E₃;
R_{N′} is —SO₂R′₁₀₀;
R and R′ are independently selected from hydrogen, —C₁-C₁₀ alkyl (optionally substituted with at least one group independently selected from OH), —C₁-C₁₀ alkylaryl, and —C₁-C₁₀ alkylheteroaryl;
R₁₀₀ and R′₁₀₀ are independently selected from
-alkoxy,
-heterocycloalkyl,
-aryl,
-heteroaryl,
-aryl-W-aryl,
-aryl-W-heteroaryl,
-aryl-W-heterocycloalkyl,
-heteroaryl-W-aryl,
-heteroaryl-W-heteroaryl,
-heteroaryl-W-heterocycloalkyl,
-heterocycloalkyl-W-aryl,
-heterocycloalkyl-W-heteroaryl,
-heterocycloalkyl-W-heterocycloalkyl,
—W—R₁₀₂,
—CH[(CH₂)₀₋₂—O—R₁₅₀]—(CH₂)₀₋₂-aryl,
—CH[(CH₂)₀₋₂—O—R₁₅₀]—(CH₂)₀₋₂-heterocycloalkyl,
—CH[(CH₂)₀₋₂—O—R₁₅₀]—(CH₂)₀₋₂-heteroaryl,
—C₁-C₁₀ alkyl optionally substituted with 1, 2, or 3 R₁₁₅ groups, wherein 1, 2, or 3 carbons of the alkyl group are optionally replaced with a group independently selected from —C(O)— and —NH—,
-alkyl-O-alkyl optionally substituted with 1, 2, or 3 R₁₁₅ groups,
-alkyl-S-alkyl optionally substituted with 1, 2, or 3 R₁₁₅ groups, and
-cycloalkyl optionally substituted with 1, 2, or 3 R₁₁₅ groups;
wherein the ring portions included within R₁₀₀ and R′₁₀₀ are optionally substituted with 1, 2, or 3 groups independently selected from —OR, —NO₂, halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄-cycloalkyl, —(CH₂)₀₋₄—R₁₁₀, —(CH₂)₀₋₄—R₁₂₀, —(CH₂)₀₋₄—R₁₃₀, —(CH₂)₀₋₄—C(O)—R₁₁₀, —(CH_{2 )}₀₋₄—C(O)—R₁₂₀, —(CH₂)₀₋₄—C(O)—R₁₃₀, —(CH₂)₀₋₄—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—O—P(O)—(O—R₁₁₀)₂, —(CH₂)₀₋₄—O—C(O)—N(R₁₅₀)₂, —(CH₂)₀₋₄—O—CS—N(R₁₅₀)₂, —(CH₂)₀₋₄—O—(R₁₅₀), —(CH₂)₀₋₄—O—R₁₅₀′—C(O)OH, —(CH₂)₀₋₄—S—(R₁₅₀), —(CH₂)₀₋₄—N(R₁₅₀)—SO₂—R₁₀₅, —(CH₂)₀₋₄-cycloalkyl, and —(C₁-C₁₀)-alkyl;
R_{E1} is selected from —H, —OH, —NH₂, —NH—(CH₂)₀₋₃—R_{E2}, —NHR_{E8}, —NR_{E350}C(O)R_{E5}, —C₁-C₄ alkyl-NHC(O)R_{E5}, —(CH₂)₀₋₄R_{E3}, —O—(C₁-C₄ alkanoyl), —C₆-C₁₀ aryloxy (optionally substituted with 1, 2, or 3 groups independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂—CF₃, —C≡—CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354};
R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10};
R_{E3} and R_{E4} are independently selected from —H, —C₁-C₃ alkyl, and —C₃-C₆ cycloalkyl;
R_{E10} is selected from alkyl, arylalkyl, alkanoyl, and arylalkanoyl;
R_{E5} is selected from cycloalkyl, alkyl (optionally substituted with 1, 2, or 3 groups independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂-C₁-C₄ alkyl, —CO₂H, —C(O)NR_{E6}R_{E7}, —CO₂—C₁-C₄ alkyl, and —C₆-C₁₀ aryloxy), heteroaryl (optionally substituted with 1, 2, or 3 groups independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7};
R_{E6} and R_{E7} are independently selected from —H, alkyl, alkanoyl, aryl, —SO₂—C₁-C₄ alkyl, and aryl-C₁-C₄ alkyl;
R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S-alkyl, and —S—C₂-C₄ alkanoyl;
R_{E9} is selected from H, alkyl, and -aryl C₁-C₄ alkyl;
R_{E350} is selected from H and alkyl;
R_{E351} is selected from aryl-(C₁-C₄ alkyl), alkyl (optionally substituted with 1, 2, or 3 groups independently selected from halogen, -cyano, -heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl;
wherein the aryl, heterocycloalkyl, —C₃-C₈ cycloalkyl, and cycloalkylalkyl groups included within R_{E351} are optionally substituted with 1, 2, 3, 4 or 5 groups. independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy;
R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —NHS(O)₀₋₂-alkyl, —N(alkyl)S(O)₀₋₂-alkyl, —S(O)₀₋₂-heteroaryl, —S(O)₀₋₂-aryl, —NH(arylalkyl), —N(alkyl)(arylalkyl), thioalkoxy, and alkoxy;
wherein each group included within R₃₅₂ is optionally substituted with 1, 2, 3, 4, or 5 groups independently selected from alkyl, alkoxy, thioalkoxy, halogen, haloalkyl, haloalkoxy, alkanoyl, —NO₂, —CN, alkoxycarbonyl, and aminocarbonyl;
R_{E353} is selected from —O—, —C(O)—, —NH—, —N(alkyl)-, —NH—S(O)₀₋₂—, —N(alkyl)—S(O)₀₋₂—, —S(O)₀₋₂—NH—, —S(O)₀₋₂—N(alkyl)-, —NH—C(S)—, and —N(alkyl)-C(S)—;
R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl;
wherein each group included within R_{E354} is optionally substituted with 1, 2, 3, 4, or 5 groups independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl;
E₁ is selected from —NR_{E11}— and —C₁-C₆ alkyl- (optionally substituted with 1, 2, or 3 groups selected from C₁-C₄ alkyl);
R_{E11} is selected from —H and alkyl; or R_{E1}, and R_{E11} combine to form —(CH₂)₁₋₄—;
E₂ is selected from a bond, —SO₂—, —SO—, —S—, and —C(O)—; and
E₃ is selected from —H, —C₁-C₄ haloalkyl, —C₅-C₆ heterocycloalkyl, —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group independently selected from halogen, alkyl, alkoxy, —CN and —NO₂), and arylalkyl (optionally substituted with at least one group independently selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, -aryl, —SO₂—C₁-C₄ alkyl, -aryl C₁-C₄ alkyl, and —C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrolidinyl;
wherein each ring is optionally substituted with 1, 2, 3, or 4 groups independently selected from alkyl, alkoxy, alkoxyalkyl, and halogen;
W is selected from —(CH₂)₀₋₄—, —O—, —S(O)₀₋₂—, —N(R₁₃₅)—, —CR(OH)—, and —C(O)—;
R₁₀₂ and R₁₀₂′ are independently selected from hydrogen and —C₁-C₁₀ alkyl (optionally substituted with 1, 2, or 3 groups independently selected from halogen, aryl, and —R₁₁₀);
R₁₀₅ and R′₁₀₅ are independently selected from
—H,
—R₁₁₀,
—R₁₂₀,
-cycloalkyl,
—(C₁-C₂ alkyl)-cycloalkyl,
-(alkyl)-O—(C₁-C₃ alkyl), and
-alkyl optionally substituted with at least one group independently selected from —OH, amine, and halogen; or
R₁₀₅ and R′₁₀₅ together with the atom to which they are attached form a 3, 4, 5, 6, or 7 membered carbocylic ring, wherein one member is optionally a heteroatom selected from —O—, —S(O)₀₋₂—, and —N(R₁₃₅)—, wherein the carbocylic ring is optionally substituted with 1, 2 or 3 R₁₄₀ groups; and
wherein the at least one carbon of the carbocylic ring is optionally replaced with —C(O)—;
R₁₁₀ is aryl optionally substituted with 1 or 2 R₁₂₅ groups;
R₁₁₅ at each occurrence is independently selected from halogen, —OH, —C(O)—O— R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂-(alkyl), alkoxy, and haloalkoxy;
R₁₂₀ is heteroaryl, optionally substituted with 1 or 2 R₁₂₅ groups;
R₁₂₅ at each occurrence is independently selected from halogen, amino, monoalkylamino, dialkylamino, —OH, —CN, —SO₂—NH₂, —SO₂—NH-alkyl, —SO₂—N(alkyl)₂, —SO₂—(C₁-C₄ alkyl), —C(O)—NH₂, —C(O)—NH-alkyl, —C(O)—N(alkyl)₂, alkyl (optionally substituted with 1, 2, or 3 groups independently selected from C₁-C₃ alkyl, halogen, —OH, —SH, —CN, —CF₃, —C₁-C₃ alkoxy, amino, monoalkylamino, and dialkylamino), and alkoxy (optionally substituted with 1, 2, or 3 halogen);
R₁₃₀ is heterocycloalkyl optionally substituted with 1 or 2 R₁₂₅ groups;
R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl);
R₁₄₀ at each occurrence is independently selected from heterocycloalkyl optionally substituted with 1, 2, 3, or 4 groups independently selected from alkyl, alkoxy, halogen, hydroxy, cyano, nitro, amino, monoalkylamino, dialkylamino, haloalkyl, haloalkoxy, amino-alkyl, monoalkylamino-alkyl, dialkylaminoalkyl, and —C(O)H;
R₁₅₀ is independently selected from
—H,
-cycloalkyl,
—(C₁-C₂ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and halogen;
R₁₅₀′ is independently selected from
-cycloalkyl,
—(C₁-C₃ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and halogen; and
R₁₈₀ is independently selected from
-morpholinyl,
-thiomorpholinyl,
-piperazinyl,
-piperidinyl,
-homomorpholinyl,
-homothiomorpholinyl,
-homothiomorpholinyl S-oxide,
-homothiomorpholinyl S,S-dioxide,
-pyrrolinyl, and
-pyrrolidinyl;
wherein each R₁₈₀ is optionally substituted with 1, 2, 3, or 4 groups independently selected from alkyl, alkoxy, halogen, hydroxy, cyano, nitro, amino, monoalkylamino, dialkylamino, haloalkyl, haloalkoxy, aminoalkyl, monoalkylamino-alkyl, dialkylamino-alkyl, and —C(O); and
wherein the at least one carbon of R₁₈₀ is optionally replaced with
—C(O)—;
R_{C} is selected from formulas (IIIa), (IIIb), and (IIIc), 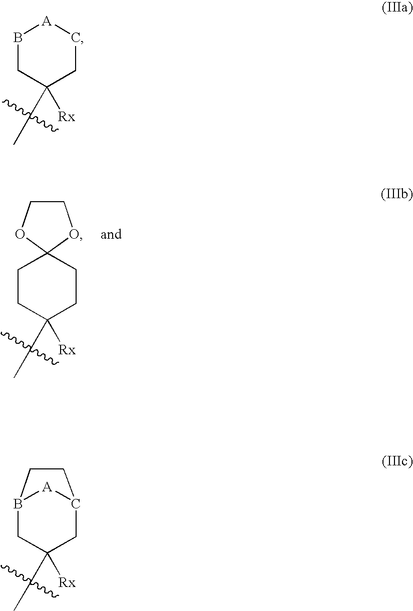
wherein,
A, B, and C are independently selected from
—CH₂—,
—O—,
—C(O)—,
—S(O)₀₋₂—,
—NH—,
—N(R₂₀₀)—,
—N(CO)₀₋₁R₂₀₀—, and
—N(S(O₂)alkyl)—;
wherein (IIIa), (IIIb), and (IIIc) are each optionally substituted with at least one group independently selected from alkyl, alkoxy, —OH, halogen, —NH₂, —NH(alkyl), —N(alkyl)(alkyl), —NH—C(O)-alkyl, and —NS(O₂)-alkyl;
Rₓ is selected from aryl, heteroaryl, cycloalkyl, heterocycloalkyl, and —Rₓₐ—R_{xb};
wherein Rₓₐ and R_{xb} are independently selected from aryl, heteroaryl, cycloalkyl, and heterocycloalkyl;
wherein each aryl or heteroaryl group within R_{C} is optionally substituted with at least one group independently selected from R₂₀₀;
wherein each cycloalkyl or heterocycloalkyl within R_{C} is optionally substituted with at least one group independently selected from R₂₁₀; and
wherein at least one carbon of the heteroaryl or heterocycloalkyl group within R_{C} is independently optionally replaced with a group selected from —NH—, —N—, —N(CO)₀₋₁R₂₁₅—, —N(CO)₀₋₁R₂₂₀—, —O—, —C(O)—, —S(O)₀₋₂—, and —NS(O)₀₋₂R₂₀₀;
R₂₀₀ at each occurrence is independently selected from
-alkyl optionally substituted with at least one group independently selected from R₂₀₅,
—OH,
—NO₂,
-halogen,
—CN,
—(CH₂)₀₋₄—C(O)H,
—(CO)₀₋₁R₂₁₅,
—(CO)₀₋₁R₂₂₀,
—(CH₂)₀₋₄—C(O)—NR₂₂₀R₂₂₅,
—(CH₂)₀₋₄—C(O)—NH(R₂₁₅),
—(CH₂)₀₋₄—C(O)-alkyl,
—(CH₂)₀₋₄—(CO)₀₋₁-cycloalkyl,
—(CH₂)₀₋₄—(CO)₀₋₁-heterocycloalkyl,
—(CH₂)₀₋₄—(CO)₀₋₁-aryl,
—(CH₂)₀₋₄—(CO)₀₋₁-heteroaryl,
—(CH₂)₀₋₄—C(O)—O—R₂₁₅,
—(CH₂)₀₋₄—SO₂—NR₂₂₀R₂₂₅,
—(CH₂)₀₋₄—S(O)₀₋₂-alkyl,
—(CH₂)₀₋₄—S(O)₀₋₂-cycloalkyl,
—(CH₂)₀₋₄—N(H or R₂₁₅)—C(O)—O—R₂₁₅,
—(CH₂)₀₋₄—N(H or R₂₁₅)—SO₂—R₂₂₀,
—(CH₂)₀₋₄—N(H or R₂₁₅)—C(O)—N(R₂₁₅)₂,
—(CH₂)₀₋₄—N(H or R₂₁₅)—C(O)—R₂₂₀,
—(CH₂)₀₋₄—NR₂₂₀R₂₂₅,
—(CH₂)₀₋₄—O—C(O)-alkyl,
—(CH₂)₀₋₄—O—(R₂₁₅),
—(CH₂)₀₋₄—S—(R₂₁₅),
—(CH₂)₀₋₄—O-alkyl optionally substituted with at least one —F, and
-adamantane;
wherein each aryl and heteroaryl group included within R₂₀₀ is optionally substituted with at least one group independently selected from R₂₀₅, R₂₁₀, and alkyl (optionally substituted with at least one group independently selected from R₂₀₅ and R₂₁₀);
wherein each cycloalkyl or heterocycloalkyl group included within R₂₀₀ is optionally substituted with at least one group independently selected from R₂₁₀; R₂₀₅ at each occurrence is independently selected from
-alkyl,
-haloalkoxy,
—(CH₂)₀₋₃-cycloalkyl,
-halogen,
—(CH₂)₀₋₆—OH,
—O-aryl,
—OH,
—SH,
—(CH₂)₀₋₄—C(O)H,
—(CH₂)₀₋₆—CN,
—(CH₂)₀₋₆—C(O)—NR₂₃₅R₂₄₀,
—(CH₂)₀₋₆—C(O)—R₂₃₅,
—(CH₂)₀₋₄—N(H or R₂₁₅)—SO₂—R₂₃₅,
—CN,
—OCF₃,
—CF₃,
-alkoxy,
-alkoxycarbonyl, and
—NR₂₃₅R₂₄₀;
R₂₁₀ at each occurrence is independently selected from
—OH,
—CN,
—(CH₂)₀₋₄—C(O)H,
-alkyl optionally substituted with at least one group independently selected from R₂₀₅,
-alkanoyl,
-halogen,
-alkoxy,
-haloalkoxy,
—NR₂₂₀R₂₂₅,
-cycloalkyl optionally substituted with at least one group independently selected from R₂₀₅,
-heterocycloalkyl,
-heteroaryl,
—(CH₂)₀₋₄—NR₂₃₅R₂₄₀,
—(CH₂)₀₋₄—NR₂₃₅(alkoxy),
—(CH₂)₀₋₄—S—(R₂₁₅),
—(CH₂)₀₋₆—OH,
—(CH₂)₀₋₆—CN,
—(CH₂)₀₋₄—NR₂₃₅—C(O)H,
—(CH₂)₀₋₄—NR₂₃₅—C(O)-(alkoxy),
—(CH₂)₀₋₄—NR₂₃₅—C(O)—R₂₄₀,
—C(O)—NHR₂₁₅,
—C(O)-alkyl,
—S(O)₂—NR₂₃₅R₂₄₀,
—C(O)—NR₂₃₅R₂₄₀, and
—S(O)₂-alkyl;
R₂₁₅ at each occurrence is independently selected from
-alkyl,
—(CH₂)₀₋₂-aryl,
—(CH₂)₀₋₂-cycloalkyl,
—(CH₂)₀₋₂-heteroaryl, and
—(CH₂)₀₋₂-heterocycloalkyl;
wherein the aryl group included within R₂₁₅ is optionally substituted with at least one group independently selected from R₂₀₅ and R₂₁₀, and
wherein the heterocycloalkyl and heteroaryl groups included within R₂₁₅ are optionally substituted with at least one group independently selected from R₂₁₀;
R₂₂₀ and R₂₂₅ at each occurrence are independently selected from
—H,
-alkyl,
—(CH₂)₀₋₄—C(O)H,
-alkylhydroxyl,
-alkoxycarbonyl,
-alkylamino,
—S(O)₂-alkyl,
-alkanoyl optionally substituted with at least one halogen,
—C(O)—NH₂,
—C(O)—NH(alkyl),
—C(O)—N(alkyl)(alkyl),
-haloalkyl,
—(CH₂)₀₋₂-cycloalkyl,
-(alkyl)-O-(alkyl),
-aryl,
-heteroaryl, and
-heterocycloalkyl;
wherein the aryl, heteroaryl, cycloalkyl, and heterocycloalkyl groups included within R₂₂₀ and R₂₂₅ are each optionally substituted with at least one group independently selected from R₂₇₀;
R₂₇₀ at each occurrence is independently selected from
—R₂₀₅,
-alkyl optionally substituted with at least one group independently selected from R₂₀₅,
-aryl,
-halogen,
-alkoxy,
-haloalkoxy,
—NR₂₃₅R₂₄₀,
—OH,
—CN,
-cycloalkyl optionally substituted with at least one group independently selected from R₂₀₅,
—C(O)-alkyl,
—S(O)₂—NR₂₃₅R₂₄₀,
—C(O)—NR₂₃₅R₂₄₀,
—S(O)₂-alkyl, and
—(CH₂)₀₋₄—C(O)H;
R₂₃₅ and R₂₄₀ at each occurrence are independently selected from
—H,
—OH,
—CF₃,
—OCH₃,
—NH—CH₃,
—N(CH₃)₂,
—(CH₂)₀₋₄—C(O)—(H or alkyl),
-alkyl,
-alkanoyl,
—SO₂-alkyl, and
-aryl.

2. The method according to claim 1, wherein U is selected from —C(O)—, —C(S)—, —S(O)₀₋₂—, —C(═NR₂₁)—, —C(═N—OR₂₁)—, —C(O)—NR₂₀—, —C(O)—O—, —S(O)₂—NR₂₀—, and —S(O)₂—O—; and V is -(T)₀₋₁-R_{N}, wherein R₂₀, R₂₁, T and R_{N} are defined as in claim 1.

3. The method according to claim 1, wherein U′ is selected from —C(O)—, —C(═NR₂₁)—, —C(═N—OR₂₁)—, —C(O)—NR₂₀—, —C(O)—O—, —S(O)₂—NR₂₀— and S(O)₂—O—; and V′ is -(T)₀₋₁-R_{N′}, wherein R₂₀, R₂₁, T and R_{N′} are defined as in claim 1.

4. The method according to claim 1, wherein U is selected from —S(O)₂—NR₂₀—, and —S(O)₂—O—, wherein R₂₀ is defined as in claim 1.

5. The method according to claim 1, wherein U is selected from —C(O)—NR₂₀—, and —C(O)—O—, wherein R₂₀ is defined as in claim 1.

6. The method according to claim 1, wherein R_{N} is 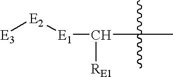
E₁ is selected from —NR_{E11}— and —C₁-C₆ alkyl-(optionally substituted with 1, 2, or 3 groups selected from C₁-C₄ alkyl);
R_{E1} is —NH₂ and R_{E11} is selected from —H and alkyl, or
R_{E1} and R_{E11} combine to form —(CH2)₁₋₄—;
E₂ is selected from a bond, —SO₂—, —SO—, —S—, and —C(O)—;
E₃ is selected from
—H,
—C₁-C₄ haloalkyl,
—C₅-C₆ heterocycloalkyl containing at least one group independently selected from N, O, and S,
—OH,
—N(E₃ₐ)(E_{3b}),
—C₁-C₁₀ alkyl optionally substituted with 1, 2, or 3 groups independently selected from halogen, hydroxy, alkoxy, thioalkoxy, and haloalkoxy,
—C₃-C₈ cycloalkyl optionally substituted with 1, 2, or 3 groups independently selected from —C₁-C₃ alkyl and halogen,
-alkoxy,
-aryl optionally substituted with at least one group independently selected from halogen, —C₁-C₄ alkyl, —C₁-C₄ alkoxy, —CN and —NO₂, and
-aryl-alkyl optionally substituted with with at least one group independently selected from halogen, alkyl, alkoxy, —CN and —NO₂;
E₃ₐ and E_{3b} are independently selected from
—H,
—C₁-C₁₀ alkyl optionally substituted with 1, 2, or 3 groups independently selected from halogen, —C₁-C₄ alkoxy, —C₃-C₈ cycloalkyl, and —OH,
—C₂-C₆ alkanoyl,
-aryl,
—SO₂—C₁-C₄ alkyl,
-aryl C₁-C₄ alkyl, and
—C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrolidinyl, wherein each ring is optionally substituted with 1, 2, 3, or 4 groups independently selected from alkyl, alkoxy, alkoxyalkyl, and halogen.

7. The method according to claim 1, wherein R_{N} is selected from alkyl, —(CH₂)₀₋₂-aryl, —C₂-C₆ alkyl, —C₂-C₆ alkyl, —C₃-C₇ cycloalkyl, and —(CH₂)₀₋₂-heteroaryl.

8. The method according to claim 1, wherein U is selected from —N(R₂₀)—C(O)— and —O—C(O)—, and R₂₀ is defined as in claim 1.

9. The method according to claim 1, wherein U is —C(O)— and T is —N(R₂₀)— or —O—, and R₂₀ is defined as in claim 1.

10. The method according to claim 1, wherein U is —C(O)— and T is —O.

11. The method according to claim 1, wherein U is —C(O)— and T is —NH—.

12. The method according to claim 1, wherein U is —SO₂— and V is -T₀₋₁-R_{N}, and R_{N} is defined as in claim 1.

13. The method according to claim 1, wherein U is selected from —C(O)— and —S(O)₀₋₂—, and V is —[C(R₄)(R₄)]₁₋₃-D, and wherein R₄, R_{4′} and D are defined as in claim 1.

14. The method according to claim 1, wherein
V is —(CH₂)₁₋₃-aryl or —(CH₂)₁₋₃-heteroaryl, wherein each ring is independently optionally substituted with 1 or 2 groups independently selected from halogen, —OH, —OCF₃, —O-phenyl, —CN, —NR₁₀₁R′₁₀₁, alkyl, alkoxy, —(CH₂)₀₋₃(C₃-C₇ cycloalkyl), aryl, heteroaryl, and heterocycloalkyl, and
wherein the alkyl, alkoxy, cycloalkyl, aryl, heteroaryl, or heterocycloalkyl groups are optionally substituted with 1 or 2 substitutents independently selected from —C₁-C₄ alkyl, —C₁-C₄ alkoxy, —C₁-C₄ haloalkyl, —C₁-C₄ haloalkoxy, halogen, —OH, —CN, and —NR₁₀₁R′₁₀₁, and wherein R₁₀₁ and R′₁₀₁ are defined as in claim 1.

15. The method according to claim 1, wherein U is —C(O)—.

16. The method according to claim 1, wherein U is selected from —C(O)— and —S(O)₀₋₂—; and V is selected from aryl, heteroaryl, cycloalkyl, and heterocycloalkyl;
wherein the aryl, heteroaryl, cycloalkyl, and heterocycloalkyl groups included within V are optionally substituted with at least one independently selected R_{B} group, and wherein R_{B} is defined as in claim 1.

17. The method according to claim 1, wherein V is selected from aryl and heteroaryl,
wherein each ring is independently optionally substituted with 1 or 2 groups independently selected from halogen, —OH, —OCF₃, —O-phenyl, —CN, —NR₁₀₁R′₁₀₁, alkyl, alkoxy, —(CH₂)₀₋₃(C₃-C₇ cycloalkyl), aryl, heteroaryl, and heterocycloalkyl, wherein the alkyl, alkoxy, cycloalkyl, aryl, heteroaryl, or heterocycloalkyl groups are optionally substituted with 1 or 2 groups independently selected from —C₁-C₄ alkyl, —C₁-C₄ alkoxy, —C₁-C₄ haloalkyl, —C₁-C₄-haloalkoxy, halogen, —OH, —CN, and —NR₁₀₁R′₁₀₁, and wherein R₁₀₁ and R′₁₀₁ are defined as in claim 1.

18. The method according to claim 1, wherein R₁ is —CH₂-phenyl, wherein the phenyl ring is optionally substituted with at least one group independently selected from halogen, C₁-C₂ alkyl, C₁-C₂ alkoxy, and —OH.

19. The method according to claim 1, wherein the compound of formula (I) is selected from
2-((4-(1-(3-tert-butylphenyl)cyclohexylamino)-1-(3,5-difluorophenyl)-3-hydroxybutan-2-ylcarbamoyl)methoxy)acetic acid,
4-(4-(1-(3-tert-butylphenyl)cyclohexylamino)-1-(3,5-difluorophenyl)-3-hydroxybutan-2-ylcarbamoyl)-2,2-dimethylbutanoic acid,
4-[3-[1-(3-tert-Butyl-phenyl)-cyclohexylamino]-1-(3,5-difluoro-benzyl)-2-hydroxy-propylcarbamoyl]-butyric acid,
N-(4-(1-(3-tert-butylphenyl)cyclohexylamino)-1-(3,5-difluorophenyl)-3-hydroxybutan-2-yl)-3-(N-methylmethan-2-ylsulfonamido)benzamide,
N-(4-(1-(3-tert-butylphenyl)cyclohexylamino)-1-(3,5-difluorophenyl)-3-hydroxybutan-2-yl)-2-(methylsulfonamido)thiazole-4-carboxamide, and
Pentanedioic acid amide [3-[1-(3-tert-butyl-phenyl)-cyclohexylamino]-1-(3,5-difluoro-benzyl)-2-hydroxy-propyl]-amide.

20. The method according to claim 1, wherein the host is a cell.

21. The method according to claim 1, wherein the host is an animal.

22. The method according to claim 1, wherein the host is human.

23. The method according to claim 1, wherein at least one compound of formula (I) is administered in combination with a pharmaceutically acceptable carrier or diluent.

24. The method according to claim 1, wherein the condition is selected from Alzheimer's disease, Down's syndrome or Trisomy 21 (including mild cognitive impairment (MCl) Down's syndrome), hereditary cerebral hemorrhage with amyloidosis of the Dutch type, chronic inflammation due to amyloidosis, prion diseases (including Creutzfeldt-Jakob disease, Gerstmann-Straussler syndrome, kuru scrapie, and animal scrapie), Familial Amyloidotic Polyneuropathy, cerebral amyloid angiopathy, other degenerative dementias, dementia associated with Parkinson's disease, dementia associated with progressive supranuclear palsy and dementia associated with cortical basal degeneration, diffuse Lewy body type of Alzheimer's disease, and frontotemporal dementias with parkinsonism (FTDP).

25. The method according to claim 1, wherein the condition is Alzheimer's disease.

26. The method according to claim 1, wherein the condition is dementia.

27. An article of manufacture, comprising:
(a) at least one dosage form of at least one compound of formula (I), 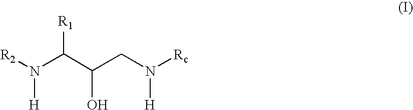
or apharmaceutically acceptable salt thereof, wherein R₁, R₂, and R_{C} are defined as in claim 1;
(b) a package insert providing that a dosage form comprising a compound of formula (I) should be administered to a patient in need of therapy for at least one disorder, condition or disease associated with amyloidosis; and
(c) at least one container in which at least one dosage form of at least one compound of formula (I) is stored.

28. A packaged pharmaceutical composition for treating at least one condition related to amyloidosis, comprising:
(a) a container which holds an effective amount of at least one compound of formula (I), 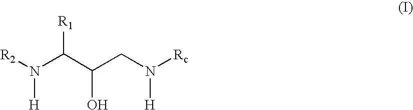
or at least one pharmaceutically acceptable salt thereof, wherein R₁, R₂, and R_{C} are defined as in claim 1; and
(b) instructions for using the pharmaceutical composition.

29. An article of manufacture, comprising:
(a) a therapeutically effective amount of at least one compound of formula (I), 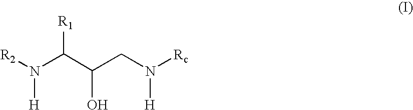
or at least one pharmaceutically acceptable salt thereof, wherein R₁, R₂, and R_{C} are defined as in claim 1;
(b) a package insert providing an oral dosage form should be administered to a patient in need of therapy for at least one disorder, condition or disease associated with amyloidosis; and
(c) at least one container comprising: at least one oral dosage form of the therapeutically effective amount at least one compound of formula (I).

30. An article of manufacture, comprising:
(a) at least one oral dosage form of at least one compound of formula (I) 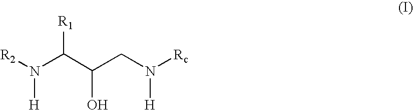
or at least one pharmaceutically acceptable salt thereof, wherein R₁, R₂, and R_{C} are defined as in claim 1;
in a dosage amount ranging from about 2 mg to about 1000 mg; associated with
(b) a package insert providing that an oral dosage form comprising: the compound of formula (I) in a dosage amount ranging from about 2 mg to about 1000 mg should be administered to a patient in need of therapy for at least one disorder, condition or disease associated with amyloidosis; and
(c) at least one container in which at least one oral dosage form of the at least one compound of formula (I) in a dosage amount ranging from about 2 mg to about 1000 mg is stored.

31. An article of manufacture, comprising:
(a) at least one oral dosage form of at least one compound of formula (I), 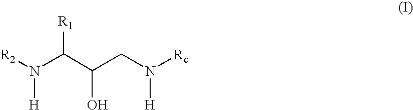
or at least one pharmaceutically acceptable salt thereof, wherein R₁, R₂, and R_{C} are defined as in claim 1, in a dosage amount ranging from about 2 mg to about 1000 mg in combination with
(b) at least one therapeutically active agent; associated with
(c) a package insert providing that an oral dosage form comprising: the compound of formula (I) in a dosage amount ranging from about 2 mg to about 1000 mg in combination with at least one therapeutically active agent should be administered to a patient in need of therapy for at least one disorder, condition or disease associated with amyloidosis; and
(d) at least one container in which at least one dosage form of the at least one compound of formula (I) in a dosage amount ranging from about 2 mg to about 1000 mg in combination with a therapeutically active agent is stored.

32. The article of manufacture according to claim 31 wherein the therapeutically active agent is selected from an antioxidant, an anti-inflammatory, a gamma-secretase inhibitor, a neurotrophic agent, an acetyl cholinesterase inhibitor, a statin, an A-beta, and an anti-A-beta antibody.

33. An article of manufacture, comprising:
(a) at least one parenteral dosage form of at least one compound of formula (I), 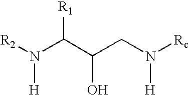
or at least one pharmaceutically acceptable salt thereof, wherein R₁, R₂, and R_{C} are defined as in claim 1, in a dosage amount ranging from about 0.2 mg/mL to about 50 mg/mL; associated with
(b) a package insert providing that a parenteral dosage form comprising: a compound of formula (I) in a dosage amount ranging from about 0.2 mg/mL to about 50 mg/mL should be administered to a patient in need of therapy for at least one disorder, condition or disease associated with amyloidosis; and
(c) at least one container in which at least one parenteral dosage form of the at least one compound of formula (I) in a dosage amount ranging from about 0.2 mg/mL to about 50 mg/mL is stored.

34. An article of manufacture comprising:
(a) a medicament comprising: an effective amount of at least one compound of formula (I), 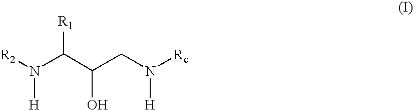
or at least one pharmaceutically acceptable salt thereof, wherein R₁, R₂, and R_{C} are defined as in claim 1, in combination with active and/or inactive pharmaceutical agents;
(b) a package insert providing that an effective amount of at least one compound of formula (I) should be administered to a patient in need of therapy for at least one disorder, condition or disease associated with amyloidosis; and
(c) a container in which a medicament comprising: an effective amount of at least one compound of formula (I) in combination with active and/or inactive pharmaceutical agents is stored.

35. A kit comprising:
(a) at least one dosage form of at least one compound according to claim 1; and
(b) at least one container in which at least one dosage form of at least one compound according to claim 1 is stored.

36. A kit according to claim 35, further comprising a package insert:
a) containing information of the dosage amount and duration of exposure of a dosage form containing at least one compound of formula (I), or at least one pharmaceutically acceptable salt thereof, and
b) providing that the dosage form should be administered to a patient in need of therapy for at least one disorder, condition or disease associated with amyloidosis.

37. A kit according to claim 36 further comprising: at least one therapeutically active agent.

38. The kit according to claim 37 wherein the therapeutically active agent is selected from an antioxidant, an anti-inflammatory, a gamma-secretase inhibitor, a neurotrophic agent, an acetyl cholinesterase inhibitor, a statin, an A-beta, and an anti-A-beta antibody.

39. A method of producing a beta-secretase complex comprising: exposing beta-secretase to a compound of formula (I), or a pharmaceutically acceptable salt thereof, in a reaction mixture under conditions suitable for the production of the complex.

40. A manufacture of a medicament for preventing, delaying, halting, or reversing Alzheimer's disease, comprising: adding an effective amount of at least one compound of formula (I), or a pharmaceutically acceptable salt thereof, to a pharmaceutically acceptable carrier.

41. A method of selecting a beta-secretase inhibitor comprising: targeting at least one moiety of at least one formula (I) compound, 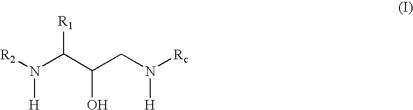
or a pharmaceutically acceptable salt thereof, wherein R₁, R₂, and R_{C} are defined as in claim 1, to interact with at least one of the following beta-secretase subsites S1, S1′, and S2′.

42. A method of preventing or treating at least one condition which benefits from inhibition of at least one aspartic-protease, comprising:
administering to a host a composition comprising a therapeutically effective amount of at least one compound of formula (I), 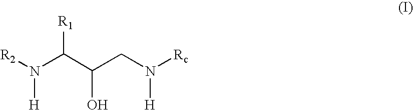
or a pharmaceutically acceptable salt thereof, wherein the inhibition is at least 10% for a dose of about 100 mg/kg or less, and wherein, wherein R₁, R₂, and R_{C} are defined as in claim 1.

43. A compound of formula (I), 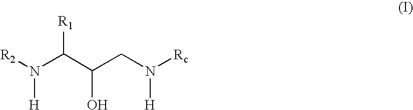
or a pharmaceutically acceptable salt thereof, wherein
R₁ is selected from 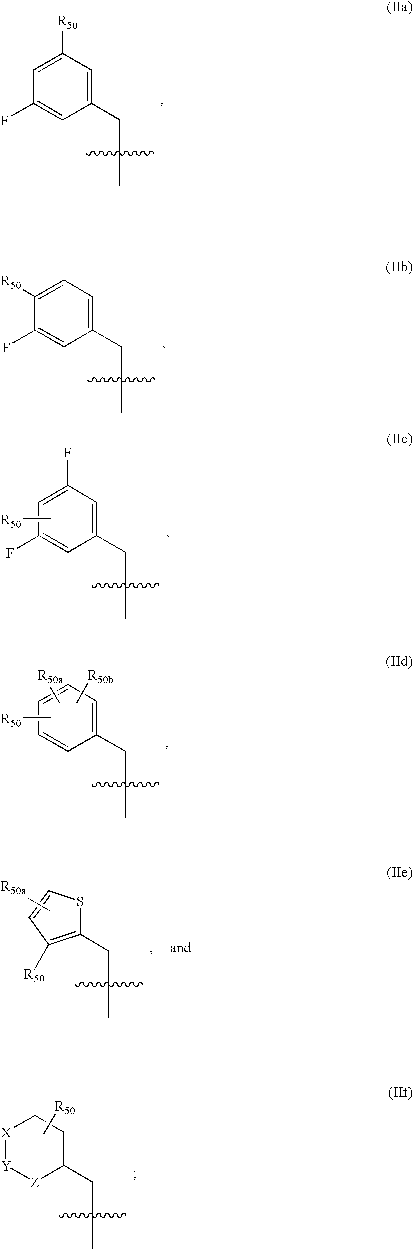
wherein
X, Y, and Z are independently selected from
—C(H)₀₋₂—,
—O—, p2 —C(O)—,
—NH—, and
—N—;
wherein at least one bond of the (IIf) ring may optionally be a double bond;
R₅₀, R₅₀ₐ, and R_{50b} are independently selected from
—H,
-halogen,
—OH,
—SH,
—CN,
—C(O)-alkyl,
—NR₇R₈,
—NO₂,
—S(O)₀₋₂-alkyl,
-alkyl,
-alkoxy,
—O-benzyl optionally substituted with at least one group independently selected from —H, —OH, and alkyl,
—C(O)—NR₇R₈,
-alkyloxy,
-alkoxyalkoxyalkoxy, and
-cycloalkyl;
wherein the alkyl, alkoxy, and cycloalkyl groups within R₅₀, R₅₀ₐ, and R_{50b} are optionally substituted with at least one group independently selected from alkyl, halogen, OH, —NR₅R₆, —CN, haloalkoxy, —NR₇R₈, and alkoxy;
R₅ and R₆ are independently selected from —H and alkyl, or
R₅ and R₆, and the nitrogen to which they are attached, form a 5 or 6 membered heterocycloalkyl ring; and
R₇ and R₈ are independently selected from
—H,
-alkyl optionally substituted with at least one group independently selected from —OH, —NH₂, and halogen,
-cycloalkyl, and
-alkyl-O-alkyl; 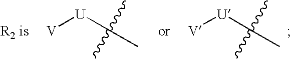
U is selected from —C(O)—, —C(═S)—, —S(O)₀₋₂—, —C(═N—R₂₁)—, —C(═N—OR₂₁)—, —C(O)—NR₂₀—, —C(O)—O—, —S(O)₂—NR₂₀—, and —S(O)₂—O—;
U′ is selected from —C(O)—, —C(═N—R₂₁)—, —C(═N—OR₂₁)—, —C(O)—NR₂₀—, and —C(O)—O—;
V is selected from aryl, heteroaryl, cycloalkyl, heterocycloalkyl, —[C(R₄)(R_{4′})]₁₋₃-D, and -(T)₀₋₁-R_{N};
V′ is selected from -(T)₀₋₁-R_{N′};
wherein the aryl, heteroaryl, cycloalkyl and heterocycloalkyl groups included within V and V′ are optionally substituted with at least one independently selected R_{B} group;
wherein at least one carbon of the aryl, heteroaryl, cycloalkyl, and heterocycloalkyl groups included within V and V′ are optionally replaced with a group selected form —C(O)—, —C(S)—, —C(═N—H)—, —C(═N—OH)—, —C(═N-alkyl)—, and —C(═N—O-alkyl)-, (CO)₀₋₁—(O)₀₋₁-alkyl, and (CO)₀₋₁—OH;
R_{B} at each occurrence is independently selected from halogen, —OH, —CF₃, —OCF₃, —O-aryl, —CN, —NR₁₀₁R′₁₀₁, alkyl, alkoxy, —(CH₂)₀₋₄—(C(O))₀₋₁—(O)₀₋₁—alkyl, —C(O)—OH, —(CH₂)₀₋₃-cycloalkyl, aryl, heteroaryl, and heterocycloalkyl; wherein the alkyl, alkoxy, cycloalkyl, aryl, heteroaryl, or heterocycloalkyl groups included within RB are optionally substituted with 1 or 2 groups independently selected from —C₁-C₄ alkyl, —C₁-C₄ alkoxy, —C₁-C₄ haloalkyl, —C₁-C₄ haloalkoxy, halogen, —OH, —CN, and —NR₁₀₁R′₁₀₁;
R₁₀₁ and R′₁₀₁ are independently selected from —H, alkyl, —(C(O))₀₋₁—(O)₀₋₁-alkyl, —C((O))₀₋₁—OH, and aryl;
R₄ and R_{4′} are independently selected from hydrogen, —OH, alkyl, —(CH₂)₀₋₃-cycloalkyl, —(CH₂)₀₋₃OH, fluorine, —CF₃, —OCF₃, —O-aryl, alkoxy, —C₃-C₇ cycloalkoxy, aryl, and heteroaryl, or
R₄ and R_{4′} are taken together with the carbon to which they are attached to form a 3, 4, 5, 6, or 7 membered carbocylic ring wherein 1, 2, or 3 carbons of the ring is optionally replaced with O, —N(H)—, —N(alkyl)—, —N(aryl)—, —C(O)—, or —S(O)₀₋₂;
D is selected from aryl, heteroaryl, cycloalkyl, and heterocycloalkyl; wherein the aryl, heteroaryl, cycloalkyl, and heterocycloalkyl are optionally substituted with 1 or 2 R_{B} groups; and
T is selected from —NR₂₀— and —O—;
R₂₀ is selected from H, —CN, alkyl, haloalkyl, and cycloalkyl;
R₂₁ is selected from —H, alkyl, haloalkyl, and cycloalkyl;
R_{N} is selected from —OH, —NH₂, —NH(alkyl), —NH(cycloalkyl), —N(alkyl)(alkyl), —N(alkyl)(cycloalkyl), —N(cycloalkyl)(cycloalkyl), —R′₁₀₀, alkyl-R₁₀₀, —(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, —(CRR′)₁₋₆—NR₁₀₀—R′₁₀₀, —(CRR′)₁₋₆—P(O)(O-alkyl)₂, alkyl-O-allkyl-C(O)OH, and —CH(R_{E1})—(CH₂)₀₋₃-E₁-E₂-E₃;
R_{N′}is —SO₂R′₁₀₀;
R and R′ are independently selected from hydrogen, —C₁-C₁₀ alkyl (optionally substituted with at least one group independently selected from OH), —C₁-C₁₀ alkylaryl, and —C₁-C₁₀ alkylheteroaryl;
R₁₀₀ and R′₁₀₀ are independently selected from
-alkoxy,
-heterocycloalkyl,
-aryl,
-heteroaryl,
-aryl-W-aryl,
-aryl-W-heteroaryl,
-aryl-W-heterocycloalkyl,
-heteroaryl-W-aryl,
-heteroaryl-W-heteroaryl,
-heteroaryl-W-heterocycloalkyl,
-heterocycloalkyl-W-aryl,
-heterocycloalkyl-W-heteroaryl,
-heterocycloalkyl-W-heterocycloalkyl,
—W—R₁₀₂,
—CH[(CH₂)₀₋₂—O—R₁₅₀]—(CH₂)₀₋₂-aryl,
—CH[(CH₂)₀₋₂—O—R₁₅₀]—(CH₂)₀₋₂-heterocycloalkyl,
—CH[(CH₂)₀₋₂—O—R₁₅₀]—(CH₂)₀₋₂-heteroaryl,
—C₁-C₁₀ alkyl optionally substituted with 1, 2, or 3 R₁₁₅ groups, wherein 1, 2, or 3 carbons of the alkyl group are optionally replaced with a group independently selected from —C(O)— and —NH—,
-alkyl-O-alkyl optionally substituted with 1, 2, or 3 R₁₁₅ groups,
-alkyl-S-alkyl optionally substituted with 1, 2, or 3 R₁₁₅ groups, and
-cycloalkyl optionally substituted with 1, 2, or 3 R₁₁₅ groups;
wherein the ring portions included within R₁₀₀ and R′₁₀₀ are optionally substituted with 1, 2, or 3 groups independently selected from —OR, —NO₂, halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂′, —(CH₂)₀₋₄—C(O)—(C₁-C₁₂ alkyl), —(CH₂)₀₋₄—C(O)—(CH₂)₀₋₄-cycloalkyl, —(CH₂)₀₋₄—R₁₁₀, —(CH₂)₀₋₄—R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R₁₃₀, —(CH₂)0-4—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)—O—C(O)-(alkyl), —(CH₂)₀₋₄—O—P(O)—(O—R₁₁₀)₂, —(CH₂)₀₋₄—O—C(O)—N(R₁₅₀)₂, —(CH₂)₀₋₄—O—CS—N(R₁₅₀)₂, —(CH₂)₀₋₄—O—(R₁₅₀), —(CH₂)₀₋₄—O—R₁₅₀′—C(O)OH, —(CH₂)₀₋₄—S—(R₁₅₀), —(CH₂)₀₋₄—N(R₁₅₀)—SO₂—R₁₀₅, —(CH₂)₀₋₄-cycloalkyl, and —(C₁-C₁₀)-alkyl;
R_{E1} is selected from —H, —OH, —NH₂, —NH—(CH₂)₀₋₃—R_{E2}, —NHR_{E8}, —NR_{E350}C(O)R_{E5}, —C₁-C₄ alkyl-NHC(O)R_{E5}, —(CH₂)₀₋₄R_{E8}, —O—(C₁-C₄ alkanoyl), —C₆-C₁₀ aryloxy (optionally substituted with 1, 2, or 3 groups independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂—CF₃, —C≡CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354};
R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10};
R_{E3} and R_{E4} are independently selected from —H, —C₁-C₃ alkyl, and —C₃-C₆ cycloalkyl;
R_{E10} is selected from alkyl, arylalkyl, alkanoyl, and arylalkanoyl;
R_{E5} is selected from cycloalkyl, alkyl (optionally substituted with 1, 2, or 3 groups independently selected from halogen, —NR_{E6}R_{E7}, C₁-C₄ alkoxy, —C₅-C₆ heterocycloalkyl, —C₅-C₆ heteroaryl, —C₆-C₁₀ aryl, -C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂—C₁-C₄ alkyl, and —C₆-C₁₀ aryloxy), heteroaryl (optionally substituted with 1, 2, or 3 groups independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7};
R_{E6} and R_{E7} are independently selected from —H, alkyl, alkanoyl, aryl, —SO₂—C₁-C₄ alkyl, and aryl-C₁-C₄ alkyl;
R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S-alkyl, and —S—C₂-C₄ alkanoyl;
R_{E9} is selected from H, alkyl, and -aryl C₁-C₄ alkyl;
R_{E350} is selected from H and alkyl;
R_{E351} is selected from aryl-(C₁-C₄ alkyl), alkyl (optionally substituted with 1, 2, or 3 groups independently selected from halogen, -cyano, -heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl;
wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy;
R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —NHS(O)₀₋₂-alkyl, —N(alkyl)S(O)₀₋₂-alkyl, —S(O)₀₋₂-heteroaryl, —S(O)₀₋₂-aryl, —NH(arylalkyl), —N(alkyl)(arylalkyl), thioalkoxy, and alkoxy;
wherein each group included within R₃₅₂ is optionally substituted with 1, 2, 3, 4, or 5 groups independently selected from alkyl, alkoxy, thioalkoxy, halogen, haloalkyl, haloalkoxy, alkanoyl, —NO₂, —CN, alkoxycarbonyl, and aminocarbonyl;
R_{E353} is selected from —O—,—C(O)—, —NH—, —N(alkyl)-, —NH—S(O)₀₋₂—, —N(alkyl)-S(O)₀₋₂—, —S(O)₀₋₂—NH—, —S(O)₀₋₂—N(alkyl)-, —NH—C(S)—, and —N(alkyl)-C(S)—;
R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl;
wherein each group included within R_{E354} is optionally substituted with 1, 2, 3, 4, or 5 groups independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl;
E₁ is selected from —NR_{E11}— and —C₁-C₆ alkyl- (optionally substituted with 1, 2, or 3 groups selected from C₁-C₄ alkyl);
R_{E11} is selected from —H and alkyl; or R_{E1} and R_{E11} combine to form —(CH₂)₁₋₄—;
E₂ is selected from a bond, —SO₂—, —SO—, —S—, and —C(O)—; and
E₃ is selected from —H, —C₁-C₄ haloalkyl, —C₅-C₆ heterocycloalkyl, —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group independently selected from halogen, alkyl, alkoxy, —CN and —NO₂), and arylalkyl (optionally substituted with at least one group independently selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, -aryl, —SO₂—C₁-C₄ alkyl, -aryl C₁-C₄ alkyl, and —C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrolidinyl;
wherein each ring is optionally substituted with 1, 2, 3, or 4 groups independently selected from alkyl, alkoxy, alkoxyalkyl, and halogen;
W is selected from —(CH₂)₀₋₄—, —O—, —S(O)₀₋₂—, —N(R₁₃₅)—, —CR(OH)—, and —C(O)—;
R₁₀₂ and R₁₀₂′ are independently selected from hydrogen and —C₁-C₁₀ alkyl (optionally substituted with 1, 2, or 3 groups independently selected from halogen, aryl, and —R₁₁₀);
R₁₀₅ and R′₁₀₅ are independently selected from
—H,
—R₁₁₀,
—R₁₂₀,
-cycloalkyl,
—(C₁-C₂ alkyl)-cycloalkyl,
-(alkyl)-O—(C₁-C₃ alkyl), and
-alkyl optionally substituted with at least one group independently selected from —OH, amine, and halogen; or
R₁₀₅ and R′₁₀₅ together with the atom to which they are attached form a 3, 4, 5, 6, or 7 membered carbocylic ring, wherein one member is optionally a heteroatom selected from —O—, —S(O)₀₋₂—, and —N(R₁₃₅)—, wherein the carbocylic ring is optionally substituted with 1, 2 or 3 R₁₄₀ groups; and wherein the at least one carbon of the carbocylic ring is optionally replaced with —C(O)—;
R₁₁₀ is aryl optionally substituted with 1 or 2 R₁₂₅ groups;
R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂-(alkyl), alkoxy, and haloalkoxy;
R₁₂₀ is heteroaryl, optionally substituted with 1 or 2 R₁₂₅ groups;
R₁₂₅ at each occurrence is independently selected from halogen, amino, monoalkylamino, dialkylamino, —OH, —CN, —SO₂—NH₂, —SO₂—NH-alkyl, —SO₂—N(alkyl)₂, —SO₂—(C₁-C₄ alkyl), —C(O)—NH₂, —C(O)—NH-alkyl, —C(O)—N(alkyl)₂, alkyl (optionally substituted with 1, 2, or 3 groups independently selected from C₁-C₃ alkyl, halogen, —OH, —SH, —CN, —CF₃, —C₁-C₃ alkoxy, amino, monoalkylamino, and dialkylamino), and alkoxy (optionally substituted with 1, 2, or 3 halogen);
R₁₃₀ is heterocycloalkyl optionally substituted with 1 or 2 R₁₂₅ groups;
R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl);
R₁₄₀ at each occurrence is independently selected from heterocycloalkyl optionally substituted with 1, 2, 3, or 4 groups independently selected from alkyl, alkoxy, halogen, hydroxy, cyano, nitro, amino, monoalkylamino, dialkylamino, haloalkyl, haloalkoxy, amino-alkyl, monoalkylamino-alkyl, dialkylaminoalkyl, and —C(O)H;
R₁₅₀ is independently selected from
—H,
-cycloalkyl,
—(C₁₂-C₂ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and halogen;
R₁₅₀′ is independently selected from
-cycloalkyl,
—(C₁-C₃ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and halogen; and
R₁₈₀ is independently selected from
-morpholinyl,
-thiomorpholinyl,
-piperazinyl,
-piperidinyl,
-homomorpholinyl,
-homothiomorpholinyl,
-homothiomorpholinyl S-oxide,
-homothiomorpholinyl S,S-dioxide,
-pyrrolinyl, and
-pyrrolidinyl;
wherein each R₁₈₀ is optionally substituted with 1, 2, 3, or 4 groups independently selected from alkyl, alkoxy, halogen, hydroxy, cyano, nitro, amino, monoalkylamino, dialkylamino, haloalkyl, haloalkoxy, aminoalkyl, monoalkylamino-alkyl, dialkylamino-alkyl, and —C(O); and
wherein the at least one carbon of R₁₈₀ is optionally replaced with —C(O)—;
R_{C} is selected from formulas (IIIa), (IIIb), and (IIIc), 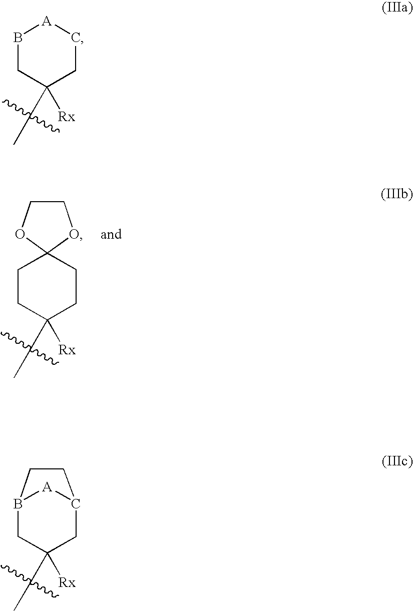
wherein,
A, B, and C are independently selected from
—CH₂—,
—O—,
—C(O)—,
—S(O)₀₋₂—,
—NH—,
—N(R₂₀₀)—,
—N(CO)₀₋₁ R₂₀₀—, and
—N(S(O₂)alkyl)-;
wherein (IIIa), (IIIb), and (IIIc) are each optionally substituted with at least one group independently selected from alkyl, alkoxy, —OH, halogen, —NH₂, —NH(alkyl), —N(alkyl)(alkyl), —NH—C(O)-alkyl, and —NS(O₂)-alkyl;
Rₓ is selected from aryl, heteroaryl, cycloalkyl, heterocycloalkyl, and —Rₓₐ—R_{xb};
wherein Rₓₐ and R_{xb} are independently selected from aryl, heteroaryl, cycloalkyl, and heterocycloalkyl;
wherein each aryl or heteroaryl group within R_{C} is optionally substituted with at least one group independently selected from R₂₀₀;
wherein each cycloalkyl or heterocycloalkyl within R_{C} is optionally substituted with at least one group independently selected from R₂₁₀; and
wherein at least one carbon of the heteroaryl or heterocycloalkyl group within R_{C} is independently optionally replaced with a group selected from —NH—, —N—, —N(CO)₀₋₁R₂₁₅—, —N(CO)₀₋₁R₂₂₀—, —O—, —C(O)—, —S(O)₀₋₂—, and —NS(O)₀₋₂R₂₀₀;
R₂₀₀ at each occurrence is independently selected from
-alkyl optionally substituted with at least one group independently selected from R₂₀₅,
—OH,
—NO₂,
-halogen,
—CN,
—(CH₂)₀₋₄—C(O)H,
—(CO)₀₋₁R₂₁₅,
—(CO)₀₋₁R₂₂₀,
—(CH₂)₀₋₄—C(O)—NR₂₂₀R₂₂₅,
—(CH₂)₀₋₄—C(O)—NH(R₂₁₅),
—(CH₂)₀₋₄—C(O)-alkyl,
—(CH₂)₀₋₄—(CO)₀₋₁-cycloalkyl,
—(CH₂)₀₋₄—(CO)₀₋₁-heterocycloalkyl,
—(CH₂)₀₋₄—(CO)₀₋₁-aryl,
—(CH₂)₀₋₄—(CO)₀₋₁-heteroaryl,
—(CH₂)₀₋₄—C(O)—O—R₂₁₅,
—(CH₂)₀₋₄—SO₂—NR₂₂₀R₂₂₅,
—(CH₂)₀₋₄—S(O)₀₋₂-alkyl,
—(CH₂)₀₋₄—S(O)₀₋₂-cycloalkyl,
—(CH₂)₀₋₄—N(H or R₂₁₅)—C(O)—O—R₂₁₅,
—(CH₂)₀₋₄—N(H or R₂₁₅)—SO₂—R₂₂₀,
—(CH₂)₀₋₄—N(H or R₂₁₅)—C(O)—N(R₂₁₅)₂,
—(CH₂)₀₋₄—N(H or R₂₁₅)—C(O)—R₂₂₀,
—(CH₂)₀₋₄—NR₂₂₀R₂₂₅,
—(CH₂)₀₋₄—O—C(O)-alkyl,
—(CH₂)₀₋₄—O—(R₂₁₅),
—(CH₂)₀₋₄—S—(R₂₁₅),
—(CH₂)₀₋₄—O-alkyl optionally substituted with at least one —F, and
-adamantane;
wherein each aryl and heteroaryl group included within R₂₀₀ is optionally substituted with at least one group independently selected from R₂₀₅, R₂₁₀, and alkyl (optionally substituted with at least one group independently selected from R₂₀₅ and R₂₁₀);
wherein each cycloalkyl or heterocycloalkyl group included within R₂₀₀ is optionally substituted with at least one group independently selected from R₂₁₀; R₂₀₅ at each occurrence is independently selected from
-alkyl,
-haloalkoxy,
—(CH₂)₀₋₃-cycloalkyl,
-halogen,
—(CH₂)₀₋₆—OH,
—O-aryl,
—OH,
—SH,
—(CH₂)₀₋₄—C(O)H,
—(CH₂)₀₋₆—CN,
—(CH₂)₀₋₆—C(O)—NR₂₃₅R₂₄₀,
—(CH₂)₀₋₆—C(O)—R₂₃₅,
—(CH₂)₀₋₄—N(H or R₂₁₅)—SO₂—R₂₃₅,
—CN,
—OCF₃,
—CF₃,
-alkoxy,
-alkoxycarbonyl, and
—NR₂₃₅R₂₄₀;
R₂₁₀ at each occurrence is independently selected from
—OH,
—CN,
—(CH₂)₀₋₄—C(O)H,
-alkyl optionally substituted with at least one group independently selected from R₂₀₅,
-alkanoyl,
-halogen,
-alkoxy,
-haloalkoxy,
—NR₂₂₀R₂₂₅,
-cycloalkyl optionally substituted with at least one group independently selected from R₂₀₅,
-heterocycloalkyl,
-heteroaryl,
—(CH₂)₀₋₄—NR₂₃₅R₂₄₀,
—(CH₂)₀₋₄—NR₂₃₅(alkoxy),
—(CH₂)₀₋₄—S—(R₂₁₅),
—(CH₂)₀₋₆—OH,
—(CH₂)₀₋₆—CN,
—(CH₂)₀₋₄—NR₂₃₅—C(O)H,
—(CH₂)₀₋₄—NR₂₃₅—C(O)-(alkoxy),
—(CH₂)₀₋₄—NR₂₃₅—C(O)—R₂₄₀,
—C(O)—NHR₂₁₅,
—C(O)-alkyl,
—S(O)₂—NR₂₃₅R₂₄₀,
—C(O)—NR₂₃₅R₂₄₀, and
—S(O)₂-alkyl;
R₂₁₅ at each occurrence is independently selected from
-alkyl,
—(CH₂)₀₋₂-aryl,
—(CH₂)₀₋₂-cycloalkyl,
—(CH₂)₀₋₂-heteroaryl,
—(CH₂)₀₋₂-heterocycloalkyl, and
—CO₂—CH₂-aryl;
wherein the aryl group included within R₂₁₅ is optionally substituted with at least one group independently selected from R₂₀₅ and R₂₁₀, and
wherein the heterocycloalkyl and heteroaryl groups included within R₂₁₅ are optionally substituted with at least one group independently selected from R₂₁₀;
R₂₂₀ and R₂₂₅ at each occurrence are independently selected from
—H,
-alkyl,
—(CH₂)₀₋₄—C(O)H,
-alkylhydroxyl,
-alkoxycarbonyl,
-alkylamino,
—S(O)₂-alkyl,
-alkanoyl optionally substituted with at least one halogen,
—C(O)—NH₂,
—C(O)—NH(alkyl),
—C(O)—N(alkyl)(alkyl),
-haloalkyl,
—(CH₂)₀₋₂-cycloalkyl,
-(alkyl)-O-(alkyl),
-aryl,
-heteroaryl, and
-heterocycloalkyl;
wherein the aryl, heteroaryl, cycloalkyl, and heterocycloalkyl groups included within R₂₂₀ and R₂₂₅ are each optionally substituted with at least one group independently selected from R₂₇₀;
R₂₇₀ at each occurrence is independently selected from
—R₂₀₅,
-alkyl optionally substituted with at least one group independently selected from R₂₀₅,
-aryl,
-halogen,
-alkoxy,
-haloalkoxy,
—NR₂₃₅R₂₄₀,
—OH,
—CN,
-cycloalkyl optionally substituted with at least one group independently selected from R₂₀₅,
—C(O)-alkyl,
—S(O)₂—NR₂₃₅R₂₄₀,
—C(O)—NR₂₃₅R₂₄₀,
—S(O)₂-alkyl, and
—(CH₂)₀₋₄—C(O)H;
R₂₃₅ and R₂₄₀ at each occurrence are independently selected from —H, —OH, —CF₃, —OCH₃, —NH—CH₃, —N(CH₃)₂, —(CH₂)₀₋₄—C(O)—(H or alkyl), alkyl, alkanoyl,
—SO₂-alkyl, and aryl.

44. A compound of formula (I), 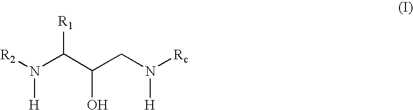
or a pharmaceutically acceptable salt thereof, wherein R₁ and R_{C} are as defined in claim 43, and wherein 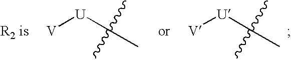
U is selected from —C(O)—, —C(═S)—, —S(O)₀₋₂—, —C(═N—R₂₁)—, —C(═N—OR₂₁)—, —C(O)—NR₂₀—, —C(O)—O—, —S(O)₂—NR₂₀—, and —S(O)₂—O—;
V is cycloalkyl (optionally substituted with at least one independently selected R_{B} group);
wherein at least one carbon of the cycloalkyl group included within V and V′ is optionally replaced with —N—, —O—, —NH—, —C(O)—, —C(S)—, —C(═N—H)—, —C(═N—OH)—, —C(═N-alkyl)-, or —C(═N—O-alkyl)-;
R_{B} at each occurrence is independently selected from halogen, —OH, —CF₃, —OCF₃, —O-aryl, —CN, —NR₁₀₁R′₁₀₁, alkyl, alkoxy, —(CH₂)₀₋₄—(C(O))₀₋₁—(O)₀₋₁-alkyl, —C(O)—OH, (CH₂)₀₋₃-cycloalkyl, aryl, heteroaryl, and heterocycloalkyl;
wherein the alkyl, alkoxy, cycloalkyl, aryl, heteroaryl, or heterocycloalkyl groups included within R_{B} are optionally substituted with 1 or 2 groups independently selected from —C₁-C₄ alkyl, —C₁-C₄ alkoxy, —C₁-C₄ haloalkyl, —C₁-C₄ haloalkoxy, halogen, —OH, —CN, and —NR₁₀₁R′₁₀₁;
R₁₀₁ and R′₁₀₁ are independently selected from —H, alkyl, —(C(O))₀₋₁—(O)₀₋₁-alkyl, —C((O))₀₋₁—OH, and aryl;
R₄ and R_{4′} are independently selected from hydrogen, —OH, alkyl, —(CH₂)₀₋₃-cycloalkyl, —(CH₂)₀₋₃OH, fluorine, —CF₃, —OCF₃, —O-aryl, alkoxy, —C₃-C₇ cycloalkoxy, aryl, and heteroaryl, or
R₄ and R_{4′} are taken together with the carbon to which they are attached to form a 3, 4, 5, 6, or 7 membered carbocylic ring wherein 1, 2, or 3 carbons of the ring is optionally replaced with O, —N(H)—, —N(alkyl)-, —N(aryl)-, —C(O)—, or —S(O)₀₋₂;
D is selected from aryl, heteroaryl, cycloalkyl, and heterocycloalkyl; wherein the aryl, heteroaryl, cycloalkyl, and heterocycloalkyl are optionally substituted with 1 or 2 R_{B} groups; and
T is selected from —NR₂₀— and —O—;
R₂₀ is selected from H, —CN, alkyl, haloalkyl, and cycloalkyl;
R₂₁ is selected from —H, alkyl, haloalkyl, and cycloalkyl;
R_{N} is selected from —OH, —NH₂, —NH(alkyl), —NH(cycloalkyl), —N(alkyl)(alkyl), —N(alkyl)(cycloalkyl), —N(cycloalkyl)(cycloalkyl), —R′₁₀₀, alkyl-R₁₀₀, —(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, —(CRR′)₁₋₆—NR₁₀₀—R′₁₀₀, —(CRR′)₁₋₆—P(O)(O-alkyl)₂, alkyl-O-allkyl-C(O)OH, and —CH(R_{E1})—(CH₂)₀₋₃-E₁-E₂-E₃;
R_{N′} is —SO₂R′₁₀₀;
R and R′ are independently selected from hydrogen, —C₁-C₁₀ alkyl (optionally substituted with at least one group independently selected from OH), —C₁-C₁₀ alkylaryl, and —C₁-C₁₀ alkylheteroaryl;
R₁₀₀ and R′₁₀₀ are independently selected from
-alkoxy,
-heterocycloalkyl,
-aryl,
-heteroaryl,
-aryl-W-aryl,
-aryl-W-heteroaryl,
-aryl-W-heterocycloalkyl,
-heteroaryl-W-aryl,
-heteroaryl-W-heteroaryl,
-heteroaryl-W-heterocycloalkyl,
-heterocycloalkyl-W-aryl,
-heterocycloalkyl-W-heteroaryl,
-heterocycloalkyl-W-heterocycloalkyl,
—W—R₁₀₂,
—CH[(CH₂)₀₋₂—O—R₁₅₀]—(CH₂)₀₋₂-aryl,
—CH[(CH₂)₀₋₂—O—R₁₅₀]—(CH₂)₀₋₂-heterocycloalkyl,
—CH[(CH₂)₀₋₂—O—R₁₅₀]—(CH₂)₀₋₂-heteroaryl,
—C₁-C₁₀ alkyl optionally substituted with 1, 2, or 3 R₁₁₅ groups, wherein 1, 2, or 3 carbons of the alkyl group are optionally replaced with a group independently selected from —C(O)— and —NH—,
-alkyl-O-alkyl optionally substituted with 1, 2, or 3 R₁₁₅ groups,
-alkyl-S-alkyl optionally substituted with 1, 2, or 3 R₁₁₅ groups, and
-cycloalkyl optionally substituted with 1, 2, or 3 R₁₁₅ groups;
wherein the ring portions included within R₁₀₀ and R′₁₀₀ are optionally substituted with 1, 2, or 3 groups independently selected from —OR, —NO₂, halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄-cycloalkyl, —(CH₂)₀₋₄R₁₁₀, —(CH₂)₀₋₄—R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R₁₃₀, —(CH₂)₀₋₄—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—O—P(O)—(O—R₁₁₀)₂, —(CH₂)₀₋₄—O—C(O)—N(R₁₅₀)₂, —(CH₂)₀₋₄—O—CS—N(R₁₅₀)₂, —(CH₂)₀₋₄—O—(R₁₅₀), —(CH₂)₀₋₄—O—R₁₅₀—C(O)OH, —CH₂)₀₋₄—S—(R₁₅₀), —(CH₂)₀₋₄—N(R₁₅₀)—SO₂—R₁₀₅, —(CH₂)₀₋₄-cycloalkyl, and —(C₁-C₁₀)-alkyl;
R_{E1} is selected from —H, —OH, —NH₂, —NH—(CH₂)₀₋₃—R_{E2}, —NHR_{E8}, —NR_{E350}C(O)R_{E5}, —C₁-C₄ alkyl-NHC(O)R_{E5}, —(CH₂)₀₋₄R_{E8}, —O—(C₁-C₄ alkanoyl), —C₆-C₁₀ aryloxy (optionally substituted with 1, 2, or 3 groups independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂—CF₃, —C≡CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₄—NHC(O)—(CH2)₀₋₆—R_{E352}, and —(CH₂)₀₋₄—(R_{E353})₀₋₁—(CH₂)₀₋₄—R_{E354};
R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10};
R_{E3} and R_{E4} are independently selected from —H, —C₁-C₃ alkyl, and —C₃-C₆ cycloalkyl;
R_{E10} is selected from alkyl, arylalkyl, alkanoyl, and arylalkanoyl;
R_{E5} is selected from cycloalkyl, alkyl (optionally substituted with 1, 2, or 3 groups independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂-C₁—C₄ alkyl, and —C₆-C₁₀ aryloxy), heteroaryl (optionally substituted with 1, 2, or 3 groups independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7};
R_{E6} and R_{E7} are independently selected from —H, alkyl, alkanoyl, aryl, —SO₂—C₁-C₄ alkyl, and aryl-C₁-C₄ alkyl;
R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S-alkyl, and —S—C₂-C₄ alkanoyl;
R_{E9} is selected from H, alkyl, and -aryl C₁-C₄ alkyl;
R_{E350} is selected from H and alkyl;
R_{E351} is selected from aryl-(C₁-C₄ alkyl), alkyl (optionally substituted with 1, 2, or 3 groups independently selected from halogen, -cyano, -heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl;
wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy;
R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —NHS(O)₀₋₂-alkyl, —N(alkyl)S(O)₀₋₂-alkyl, —S(O)₀₋₂-heteroaryl, —S(O)₀₋₂-aryl, —NH(arylalkyl), —N(alkyl)(arylalkyl), thioalkoxy, and alkoxy;
wherein each group included within R₃₅₂ is optionally substituted with 1, 2, 3, 4, or 5 groups independently selected from alkyl, alkoxy, thioalkoxy, halogen, haloalkyl, haloalkoxy, alkanoyl, —NO₂, —CN, alkoxycarbonyl, and aminocarbonyl;
R_{E353} is selected from —O—, —C(O)—, —NH—, —N(alkyl)-, —NH—S(O)₀₋₂—, —N(alkyl)-S(O)₀₋₂—, —S(O)₀₋₂—NH—, —S(O)₀₋₂—N(alkyl)-, —NH—C(S)—, and —N(alkyl)-C(S)—;
R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl;
wherein each group included within R_{E354} is optionally substituted with 1, 2, 3, 4, or 5 groups independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl;
E₁ is selected from —NR_{E11}— and —C₁-C₆ alkyl- (optionally substituted with 1, 2, or 3 groups selected from C₁-C₄ alkyl);
R_{E11} is selected from —H and alkyl; or R_{E1} and R_{E11} combine to form —(CH₂)₁₋₄—;
E₂ is selected from a bond, —SO₂—, —SO—, —S—, and —C(O)—; and
E₃ is selected from —H, —C₁-C₄ haloalkyl, —C₅-C₆ heterocycloalkyl, —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group independently selected from halogen, alkyl, alkoxy, —CN and —NO₂), and arylalkyl (optionally substituted with at least one group independently selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, -aryl, —SO₂—C₁-C₄ alkyl, -aryl C₁-C₄ alkyl, and —C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrolidinyl;
wherein each ring is optionally substituted with 1, 2, 3, or 4 groups independently selected from alkyl, alkoxy, alkoxyalkyl, and halogen;
W is selected from —(CH₂)₀₋₄—, —O—, —S(O)₀₋₂—, —N(R₁₃₅)—, —CR(OH)—, and —C(O)—;
R₁₀₂ and R₁₀₂′ are independently selected from hydrogen and —C₁-C₁₀ alkyl (optionally substituted with 1, 2, or 3 groups independently selected from halogen, aryl, and —R₁₁₀);
R₁₀₅ and R′₁₀₅ are independently selected from
—H,
—R₁₁₀,
—R₁₂₀,
-cycloalkyl,
—(C₁-C₂ alkyl)-cycloalkyl,
-(alkyl)-O—(C₁-C₃ alkyl), and
-alkyl optionally substituted with at least one group independently selected from —OH, amine, and halogen; or
R₁₀₅ and R′₁₀₅ together with the atom to which they are attached form a 3, 4, 5, 6, or 7 membered carbocylic ring, wherein one member is optionally a heteroatom selected from —O—, —S(O)₀₋₂—, and —N(R₁₃₅)—, wherein the carbocylic ring is optionally substituted with 1, 2 or 3 R₁₄₀ groups; and wherein the at least one carbon of the carbocylic ring is optionally replaced with —C(O)—;
R₁₁₀ is aryl optionally substituted with 1 or 2 R₁₂₅ groups;
R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂-(alkyl), alkoxy, and haloalkoxy;
R₁₂₀ is heteroaryl, optionally substituted with 1 or 2 R₁₂₅ groups;
R₁₂₅ at each occurrence is independently selected from halogen, amino, monoalkylamino, dialkylamino, —OH, —CN, —SO₂—NH₂, —SO₂—NH-alkyl, —SO₂—N(alkyl)₂, —SO₂—(C₁-C₄ alkyl), —C(O)—NH₂, —C(O)—NH-alkyl, —C(O)—N(alkyl)₂, alkyl (optionally substituted with 1, 2, or 3 groups independently selected from C₁-C₃ alkyl, halogen, —OH, —SH, —CN, —CF₃, —C₁-C₃ alkoxy, amino, monoalkylamino, and dialkylamino), and alkoxy (optionally substituted with 1, 2, or 3 halogen);
R₁₃₀ is heterocycloalkyl optionally substituted with 1 or 2 R₁₂₅ groups;
R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl);
R₁₄₀ at each occurrence is independently selected from heterocycloalkyl optionally substituted with 1, 2, 3, or 4 groups independently selected from alkyl, alkoxy, halogen, hydroxy, cyano, nitro, amino, monoalkylamino, dialkylamino, haloalkyl, haloalkoxy, amino-alkyl, monoalkylamino-alkyl, dialkylaminoalkyl, and —C(O)H;
R₁₅₀ is independently selected from
—H,
-cycloalkyl,
—(C₁-C₂ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and halogen;
R₁₅₀′ is independently selected from
-cycloalkyl,
—(C₁-C₃ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and halogen; and
R₁₈₀ is independently selected from
-morpholinyl,
-thiomorpholinyl,
-piperazinyl,
-piperidinyl,
-homomorpholinyl,
-homothiomorpholinyl,
-homothiomorpholinyl S-oxide,
-homothiomorpholinyl S,S-dioxide,
-pyrrolinyl, and
-pyrrolidinyl;
wherein each R₁₈₀ is optionally substituted with 1, 2, 3, or 4 groups independently selected from alkyl, alkoxy, halogen, hydroxy, cyano, nitro, amino, monoalkylamino, dialkylamino, haloalkyl, haloalkoxy, aminoalkyl, monoalkylamino-alkyl, dialkylamino-alkyl, and —C(O); and
wherein the at least one carbon of R₁₈₀ is optionally replaced with —C(O)—.

45. A compound of formula (I) 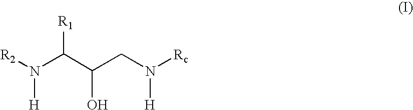
or a pharmaceutically acceptable salt thereof, wherein the formula (I) compound is selected from
cyclopent-1-enecarboxylic acid [3-[1-(3-tert-butyl-phenyl)-cyclohexylamino]-1-(3,5-difluoro-benzyl)-2-hydroxy-propyl]-amide,
cyclopropanecarbothioic acid [3-[1-(3-tert-butyl-phenyl)-cyclohexylamino]-1-(3,5-difluoro-benzyl)-2-hydroxy-propyl]-amide,
2-Oxo-imidazolidine-4-carboxylic acid [3-[1-(3-tert-butyl-phenyl)-cyclohexylamino]-1-(3,5-difluoro-benzyl)-2-hydroxy-propyl]-amide,
3-Acetylamino-N-[3-[1-(3-tert-butyl-phenyl)-cyclohexylamino]-1-(3,5-difluoro-benzyl)-2-hydroxy-propyl]-propionamide,
5-Acetylamino-pentanoic acid [3-[1-(3-tert-butyl-phenyl)-cyclohexylamino]-1-(3,5-difluoro-benzyl)-2-hydroxy-propyl]-amide,
1-[3-[1-(3-tert-Butyl-phenyl)-cyclohexylamino]-1-(3,5-difluoro-benzyl)-2-hydroxy-propyl]-3-(3,5-dimethyl-isoxazol-4-yl)-urea,
3-{3-[3-[1-(3-tert-Butyl-phenyl)-cyclohexylamino]-1-(3,5-difluoro-benzyl)-2-hydroxy-propyl]-ureido}-propionic acid ethyl ester,
2-{3-[3-[1-(3-tert-Butyl-phenyl)-cyclohexylamino]-1-(3,5-difluoro-benzyl),-2-hydroxy-propyl]-ureido}-3-methyl-butyric acid ethyl ester,
2-{3-[3-[1-(3-tert-Butyl-phenyl)-cyclohexylamino]-1-(3,5-difluoro-benzyl)-2-hydroxy-propyl]-ureido}-4-methyl-pentanoic acid ethyl ester,
N-[3-[1-(3-tert-Butyl-phenyl)-cyclohexylamino]-1-(3,5-difluoro-benzyl)-2-hydroxy-propyl]-4-sulfamoyl-butyramide,
1-Methyl-cyclopropanecarboxylic acid [3-[1-(3-tert-butyl-phenyl)-cyclohexylamino]-1-(3,5-difluoro-benzyl)-2-hydroxy-propyl]-amide,
tert-butyl (4-(1-(3-tert-butylphenyl)cyclohexylamino)-1-(3,5-difluorophenyl)-3-hydroxybutan-2-ylcarbamoyl)methylcarbamate,
4,7,7-Trimethyl-3-oxo-2-oxa-bicyclo[2.2.1 ]heptane-1-carboxylic acid [3-[1-(3-tert-butyl-phenyl)-cyclohexylamino]-1-(3,5-difluoro-benzyl)-2-hydroxy-propyl]-amide,
{[3-[1-(3-tert-Butyl-phenyl)-cyclohexylamino]-1-(3,5-difluoro-benzyl)-2-hydroxy-propylcarbamoyl]-methyl}-phosphonic acid diethyl ester,
N-[3-[1-(3-tert-Butyl-phenyl)-cyclohexylamino]-1-(3,5-difluoro-benzyl)-2-hydroxy-propyl]-2-(2,5-dioxo-imidazolidin-4-yl)-acetamide,
(E)-N-(4-(1-(3-tert-butylphenyl)cyclohexylamino)-1-(3,5-difluorophenyl)-3-hydroxybutan-2-yl)-3-(pyridin-3-yl)acrylamide,
N-(4-(1-(3-tert-butylphenyl)cyclohexylamino)-1-(3,5-difluorophenyl)-3-hydroxybutan-2-yl)-2-oxothiazolidine-4-carboxamide,
tert-butyl 3-(4-(1-(3-tert-butylphenyl)cyclohexylamino)-1-(3,5-difluorophenyl)-3-hydroxybutan-2-ylcarbamoyl)azetidine-1-carboxylate,
5-Oxo-tricyclo[2.2.1.02,6]heptane-3-carboxylic acid [3-[1-(3-tert-butyl-phenyl)-cyclohexylamino]-1-(3,5-difluoro-benzyl)-2-hydroxy-propyl]-amide,
N-(4-(1-(3-tert-butylphenyl)cyclohexylamino)-1-(3,5-difluorophenyl)-3-hydroxybutan-2-yl)-5-oxopyrrolidine-2-carboxamide,
2-((4-(1-(3-tert-butylphenyl)cyclohexylamino)-1-(3,5-difluorophenyl)-3-hydroxybutan-2-ylcarbamoyl)methoxy)acetic acid,
3-(4-(1-(3-tert-butylphenyl)cyclohexylamino)-1-(3,5-difluorophenyl)-3-hydroxybutan-2-ylcarbamoyl)cyclohexanecarboxylic acid,
methyl 4-(4-(1-(3-tert-butylphenyl)cyclohexylamino)-1-(3,5-difluorophenyl)-3-hydroxybutan-2-ylcarbamoyl)-4-methylpentanoate,
1-(2-amino-2-oxoethyl)-N-(4-(1-(3-tert-butylphenyl)cyclohexylamino)-1-(3,5-difluorophenyl)-3-hydroxybutan-2-yl)pyrrolidine-2-carboxamide,
tert-butyl 4-(tert-butoxycarbonyl)-5-(4-(1-(3-tert-butylphenyl)cyclohexylamino)-1-(3,5-difluorophenyl)-3-hydroxybutan-2-ylamino)-5-oxopentanoate,
1-(4-(1-(3-tert-butylphenyl)cyclohexylamino)-1-(3,5-difluorophenyl)-3-hydroxybutan-2-yl)urea,
N-(4-(1-(3-tert-butylphenyl)cyclohexylamino)-1-(3,5-difluorophenyl)-3-hydroxybutan-2-yl)-3-oxo-2-oxa-bicyclo[2.2.1 ]heptane-1-carboxamide,
5-Oxo-pyrrolidine-2-carboxylic acid [3-[1-(3-tert-butyl-phenyl)-cyclohexylamino]-1-(3,5-difluoro-benzyl)-2-hydroxy-propyl]-amide,
ethyl 2-(3-(4-(1-(3-tert-butylphenyl)cyclohexylamino)-1-(3,5-difluorophenyl)-3-hydroxybutan-2-yl)ureido)-4-(methylthio)butanoate, and
N-[3-[1-(3-tert-Butyl-phenyl)-cyclohexylamino]-1-(3,5-difluoro-benzyl)-2-hydroxy-propyl]-2-(2-imino-imidazolidin-1-yl)-acetamide.


SEQUENCE LISTING (11 sequences)

SEQ ID NO: 1 - protein, 4 aa, Artificial Sequence
  Description of Artificial Sequence Synthetic peptide
KMDA

SEQ ID NO: 2 - protein, 4 aa, Artificial Sequence
  Description of Artificial Sequence Synthetic peptide
NLDA

SEQ ID NO: 3 - protein, 13 aa, Artificial Sequence
  Description of Artificial Sequence Synthetic peptide
SEVNLDAEFRCKK

SEQ ID NO: 4 - protein, 13 aa, Artificial Sequence
  Description of Artificial Sequence Synthetic peptide
SEVKMDAEFRCKK

SEQ ID NO: 5 - protein, 22 aa, Artificial Sequence
  Description of Artificial Sequence Synthetic peptide
GLNIKTEEISEISYEVEFRCKK

SEQ ID NO: 6 - protein, 34 aa, Artificial Sequence
  Description of Artificial Sequence Synthetic peptide
ADRGLTTRPGSGLTNIKTEEISEVNLDAEFRCKK

SEQ ID NO: 7 - protein, 33 aa, Artificial Sequence
  Description of Artificial Sequence Synthetic peptide
FVNQHLCGSHLVEALYLVCGERGFFYTPKACKK

SEQ ID NO: 8 - protein, 33 aa, Artificial Sequence
  Description of Artificial Sequence Synthetic peptide
CGGADRGLTTRPGSGLTNIKTEEISEVNLDAEF

SEQ ID NO: 9 - protein, 29 aa, Artificial Sequence
  Description of Artificial Sequence Synthetic peptide
CGGADRGLTTRPGSGLTNIKTEEISEVNL

SEQ ID NO: 10 - protein, 9 aa, Artificial Sequence
  Description of Artificial Sequence Synthetic peptide
SEVNLDAEF

SEQ ID NO: 11 - protein, 30 aa, Artificial Sequence
  Description of Artificial Sequence Synthetic peptide
ADRGLTTRPGSGLTNIKTEEISEVNLDAEF